SEQUENCE LISTING (71 sequences)

SEQ ID NO: 1 - protein, 7 aa, Simian virus 40
PKKKRKV

SEQ ID NO: 2 - protein, 16 aa, Artificial Sequence
  Synthetic nucleoplasmin bipartite NLS
KRPAATKKAGQAKKKK

SEQ ID NO: 3 - protein, 9 aa, Homo sapiens
PAAKRVKLD

SEQ ID NO: 4 - protein, 11 aa, Homo sapiens
RQRRNELKRSP

SEQ ID NO: 5 - protein, 38 aa, Homo sapiens
NQSSNFGPMKGGNFGGRSSGPYGGGGQYFAKPRNQGGY

SEQ ID NO: 6 - protein, 42 aa, Homo sapiens
RMRIZFKNKGKDTAELRRRRVEVSVELRKAKKDEQILKRRNV

SEQ ID NO: 7 - protein, 8 aa, Homo sapiens
VSRKRPRP

SEQ ID NO: 8 - protein, 8 aa, Homo sapiens
PPKKARED

SEQ ID NO: 9 - protein, 8 aa, Homo sapiens
PQPKKKPL

SEQ ID NO: 10 - protein, 12 aa, Mus sp.
SALIKKKKKMAP

SEQ ID NO: 11 - protein, 5 aa, Influenza virus
DRLRR

SEQ ID NO: 12 - protein, 7 aa, Influenza virus
PKQKKRK

SEQ ID NO: 13 - protein, 10 aa, Hepatitis D virus
RKLKKKIKKL

SEQ ID NO: 14 - protein, 10 aa, Mus sp.
REKKKFLKRR

SEQ ID NO: 15 - protein, 20 aa, Homo sapiens
KRKGDEVDGVDEVAKKKSKK

SEQ ID NO: 16 - protein, 17 aa, Homo sapiens
RKCLQAGMNLEARKTKK

SEQ ID NO: 17 - protein, 15 aa, Artificial Sequence
  GlySer Linker
GGGGSGGGGSGGGGS

SEQ ID NO: 18 - protein, 4 aa, Artificial Sequence
  GlySer Linker
GGGS

SEQ ID NO: 19 - protein, 12 aa, Artificial Sequence
  GlySer Linker
GGGSGGGSGGGS

SEQ ID NO: 20 - protein, 7 aa, Artificial Sequence
  GlySer Linker
AGAAAKA

SEQ ID NO: 21 - protein, 30 aa, Artificial Sequence
  GlySer Linker
GGGGSGGGGSGGGGSGGGGSGGGGSGGGGS

SEQ ID NO: 22 - protein, 45 aa, Artificial Sequence
  GlySer Linker
GGGGSGGGGSGGGGSGGGGSGGGGSGGGGSGGGGSGGGGSGGGGS

SEQ ID NO: 23 - protein, 60 aa, Artificial Sequence
  GlySer Linker
GGGGSGGGGSGGGGSGGGGSGGGGSGGGGSGGGGSGGGGSGGGGSGGGGSGGGGSGGGGS

SEQ ID NO: 24 - protein, 5 aa, Artificial Sequence
  GlySer Linker
GGGGS

SEQ ID NO: 25 - protein, 10 aa, Artificial Sequence
  GlySer Linker
GGGGSGGGGS

SEQ ID NO: 26 - protein, 20 aa, Artificial Sequence
  GlySer Linker
GGGGSGGGGSGGGGSGGGGS

SEQ ID NO: 27 - protein, 25 aa, Artificial Sequence
  GlySer Linker
GGGGSGGGGSGGGGSGGGGSGGGGS

SEQ ID NO: 28 - protein, 35 aa, Artificial Sequence
  GlySer Linker
GGGGSGGGGSGGGGSGGGGSGGGGSGGGGSGGGGS

SEQ ID NO: 29 - protein, 40 aa, Artificial Sequence
  GlySer Linker
GGGGSGGGGSGGGGSGGGGSGGGGSGGGGSGGGGSGGGGS

SEQ ID NO: 30 - protein, 50 aa, Artificial Sequence
  GlySer Linker
GGGGSGGGGSGGGGSGGGGSGGGGSGGGGSGGGGSGGGGSGGGGSGGGGS

SEQ ID NO: 31 - protein, 55 aa, Artificial Sequence
  GlySer Linker
GGGGSGGGGSGGGGSGGGGSGGGGSGGGGSGGGGSGGGGSGGGGSGGGGSGGGGS

SEQ ID NO: 32 - protein, 107 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 33 - protein, 9 aa, Homo sapiens
IEVMYPPPY

SEQ ID NO: 34 - DNA, 3431 bp, Artificial Sequence
  Cas9t-1 contig
ctaccccgcaaggaaggagccgccgaccgatgtcttgttaggaaaactaatccttcggaaattagtcgagaaggggctcgcccccccaaaagccaacctgtcgtggcaatgcatcgaaagcctgaccatcaccccgtggatatgggatcgcttcgccgagcacgggatcgaagtcgcgctcggcgagatggtcaggcggcggggcgaagagaaatcaaaaaaaaatcgaacggggggttgacttttggataatagccttcataatctcgcccatcgatagccgagcaacgaagttggatcaggacgtcattcttcgaattcgaaaacgcggggattgctcgaatgggtggatgatatgttggatcaggacgtcacttttcgaattcgaaaacgcaagtcgaagttggcgacttgatcaaatagttggatcaggacgtcacttttcgaattcgaaaactaaagaccgctcggctgatacttgacggaagttggatcaggacgtcacttttcgaattcgaaaaccgatctcgcctaagtgattgattcagcgaagaaattgtagaatcgcagttgcaaaaaatgatgtcctgatccaataaggggcgcagcccccgcaccatttttccccgatctacggatcggatcggcgctgccttcgaaaagccgacatggaaaacccccagtttatcggggacgatgtaaacctgagtcgccacggcgctcatctcgcccgctctacggaccgggcttcgctggaaactgggagcggtaaccatgaaacaactcacactcgatgtcggcatcgcttcgatcggctgggcgattgtcagtaaaaaagggaacgtcaaagccggatcacgcatctttcccgacgccaaatctgggcgggaaagcaaccaatcccgacgggcggctcggctgatgcgccgaggatatcggcgcaaagcaaagcgccgagccgacacgctcgccatcattcgctccatccaccccggcttcgaccccgaagggcaccctgacatcgagcgcgaagctctgatcaaagccatcattacccccggcgcccccaccccatcgctcgaccaactcgcttgcgcgttccagcgattcgccaagtctcggtggccgcaatattcgagaactctcccaaaacgaaccgagcgggaagaccagttcatccaagtttgggtgatcgccgagcgaacctatcccgaccgctttacccccgacgtcgcaacccgcttgcttcaggcgatattctttcagcgccctatcaaagacggcgaccgcgcgaaatgtcaactattcaggcatcacggtgacaaagccccgctcgtcggctggacgcacgaacccgaactccaacgattcgccatcttgtccgatctgtccaatctcactatcgggatcggctcgactgataacctgctttgcgaatatcccgacatcatcgaagacttggagacccgatgcttcgagaccggcatgagctggagagagatagccgaacacgtcaaggaagtcatcggcaaaggggtggtgtttcgaggcattgacgggcagaaaaaagtcgggcggaacgggatcgggcctgccaaactcgaaacaatcgacgaagaaggcaactctaccaagagcaccgcttcgatgtcggtcgaagcggcggtaatgatctatcaccaaatgaaagcggatcgatgccgagcggcaaccgctaaaaaaacgctgatcgacgcaggggctctttcagcccccctaaccgccaaagatatcaaacgtggcgatcgcactctcaccatcaccgaactgatggatatggcgggcaggattaccgacccgactatcagggcgatctaccaccaagtcgagatgttggtcaacgagttgatcgcccgcttcggtaaacccgaacgcatcgttatcgaagcccaaaaggaaatcgggcgaagcatcgaagacatcgagaaagcgatggcaagagagcgagaaaagcatatcgaacggcaacgagagaatcgagcccgcaacgccgcaatgggtaccaaagcccgctttgcccgcctgtgcgccatcaggggtgatcgatgctttatcagcgaccgacccgcagccgaagtcggccacctgatcgccgattcgatcgggggcacgcttgaaatggctaacctgatcccgatcgaccctgccatcaataaagagatgggcaaccgcactccctacgaagcctttcgaaagactgagtattggtcgatcatccagcgcaaactgcaagcgcttgaagacgaagtgaaggctttgaaaccgccgaaagggacaaaaggaacggcgtggacgatctatcaccgagccaagcatcagtttgattttttcgcttggcgatttcaatcgaatgcgagagaaacccatcaaaggaactttcgccccggctcgcttgacgacctgcggtggatcgaaaacttgctctttctcggcgttgccccgatttgcgacaatatccgaatcgtcagtgggcgaacaaccgagcgcattcgccgagagatacttggaatggacaaagaccgccgagaccatcgccaccatgcgctcgatgcgcttgctatcatgctcgccaatcctttgaagccgtgggatttgaaatcgagcaattcgctcggtatcccgctcggccgaatcaaacaagccttcgccgacgctgtcgtctcgcaaaagcaagaccactcgcttcgcactgcgttgcataaagagaacgcgatcccgaagaccaagcggggggctgcatatcgaaaaatcggaacgggggcgagcgaacgcgtagtcgacacccaatcaaaggcctattgcgaagtttgggcgttgcctaacgggaagtgggaagcggtcgtagtgtcgagtttcgacgctgcgcaaaagaactaccggcaagggattgaccatcgcccccatcccgccgctcgactggttatgcgcttgttcaaatccgatttgctcggtatcgggggcaagatataccgggttcaggaactactcggttctggctcgatctacttggtcgatcatcgattcgctggcactattcgagacgcccgcgcagtttgtaagacgggcgtcaatgtcgatttctttagcaaagggggtgattcattgcgcaaggcgggggctcgtcttgtttcgattcgtaagagttgggtgggatcgtgagtcacttttcgaattcggaaacttttccgaccgacaagtactcgatcatctagttggatcaggacgtcatgcttcgaattcgaaaacgtcagtcgggtgacctacgagcgatagggggttggatcaggacgtcacttttcgaattcgaaaaccattcgatgccgatgcctacaccgcaaagagttggatcaggacgtcatgcttcgaattcgaaaaccttcgtttcaataacc

SEQ ID NO: 35 - DNA, 2436 bp, Artificial Sequence
  Cas9t-1 contig for synthesis
ctgggagcggtaaccatgaaacaactcacactcgatgtcggcatcgcttcgatcggctgggcgattgtcagtaaaaaagggaacgtcaaagccggatcacgcatctttcccgacgccaaatctgggcgggaaagcaaccaatcccgacgggcggctcggctgatgcgccgaggatatcggcgcaaagcaaagcgccgagccgacacgctcgccatcattcgctccatccaccccggcttcgaccccgaagggcaccctgacatcgagcgcgaagctctgatcaaagccatcattacccccggcgcccccaccccatcgctcgaccaactcgcttgcgcgttccagcgattcgccaagtctcggtggccgcaatattcgagaactctcccaaaacgaaccgagcgggaagaccagttcatccaagtttgggtgatcgccgagcgaacctatcccgaccgctttacccccgacgtcgcaacccgcttgcttcaggcgatattctttcagcgccctatcaaagacggcgaccgcgcgaaatgtcaactattcaggcatcacggtgacaaagccccgctcgtcggctggacgcacgaacccgaactccaacgattcgccatcttgtccgatctgtccaatctcactatcgggatcggctcgactgataacctgctttgcgaatatcccgacatcatcgaagacttggagacccgatgcttcgagaccggcatgagctggagagagatagccgaacacgtcaaggaagtcatcggcaaaggggtggtgtttcgaggcattgacgggcagaaaaaagtcgggcggaacgggatcgggcctgccaaactcgaaacaatcgacgaagaaggcaactctaccaagagcaccgcttcgatgtcggtcgaagcggcggtaatgatctatcaccaaatgaaagcggatcgatgccgagcggcaaccgctaaaaaaacgctgatcgacgcaggggctctttcagcccccctaaccgccaaagatatcaaacgtggcgatcgcactctcaccatcaccgaactgatggatatggcgggcaggattaccgacccgactatcagggcgatctaccaccaagtcgagatgttggtcaacgagttgatcgcccgcttcggtaaacccgaacgcatcgttatcgaagcccaaaaggaaatcgggcgaagcatcgaagacatcgagaaagcgatggcaagagagcgagaaaagcatatcgaacggcaacgagagaatcgagcccgcaacgccgcaatgggtaccaaagcccgctttgcccgcctgtgcgccatcaggggtgatcgatgctttatcagcgaccgacccgcagccgaagtcggccacctgatcgccgattcgatcgggggcacgcttgaaatggctaacctgatcccgatcgaccctgccatcaataaagagatgggcaaccgcactccctacgaagcctttcgaaagactgagtattggtcgatcatccagcgcaaactgcaagcgcttgaagacgaagtgaaggctttgaaaccgccgaaagggacaaaaggaacggcgtggacgatctatcaccgagccaagcatcagtttgattttttcgcttggcgatttcaatcgaatgcgagagaaacccatcaaaggaactttcgccccggctcgcttgacgacctgcggtggatcgaaaacttgctctttctcggcgttgccccgatttgcgacaatatccgaatcgtcagtgggcgaacaaccgagcgcattcgccgagagatacttggaatggacaaagaccgccgagaccatcgccaccatgcgctcgatgcgcttgctatcatgctcgccaatcctttgaagccgtgggatttgaaatcgagcaattcgctcggtatcccgctcggccgaatcaaacaagccttcgccgacgctgtcgtctcgcaaaagcaagaccactcgcttcgcactgcgttgcataaagagaacgcgatcccgaagaccaagcggggggctgcatatcgaaaaatcggaacgggggcgagcgaacgcgtagtcgacacccaatcaaaggcctattgcgaagtttgggcgttgcctaacgggaagtgggaagcggtcgtagtgtcgagtttcgacgctgcgcaaaagaactaccggcaagggattgaccatcgcccccatcccgccgctcgactggttatgcgcttgttcaaatccgatttgctcggtatcgggggcaagatataccgggttcaggaactactcggttctggctcgatctacttggtcgatcatcgattcgctggcactattcgagacgcccgcgcagtttgtaagacgggcgtcaatgtcgatttctttagcaaagggggtgattcattgcgcaaggcgggggctcgtcttgtttcgattcgtaagagttgggtgggatcg

SEQ ID NO: 36 - DNA, 3236 bp, Artificial Sequence
  Cas96-1 Contig for synthesis
ctaccccgcaaggaaggagccgccgaccgatgtcttgttaggaaaactaatccttcggaaattagtcgagaaggggctcgcccccccaaaagccaacctgtcgtggcaatgcatcgaaagcctgaccatcaccccgtggatatgggatcgcttcgccgagcacgggatcgaagtcgcgctcggcgagatggtcaggcggcggggcgaagagaaatcaaaaaaaaatcgaacggggggttgacttttggataatagccttcataatctcgcccatcgatagccgagcaacgaagttggatcaggacgtcattcttcgaattcgaaaacgcggggattgctcgaatgggtggatgatatgttggatcaggacgtcacttttcgaattcgaaaaccgatctcgcctaagtgattgattcagcgaagaaattgtagaatcgcagttgcaaaaaatgatgtcctgatccaataaggggcgcagcccccgcaccatttttccccgatctacggatcggatcggcgctgccttcgaaaagccgacatggaaaacccccagtttatcggggacgatgtaaacctgagtcgccacggcgctcatctcgcccgctctacggaccgggcttcgctggaaaatgggagccgtgaccatgaagcagctgaccctggatgtgggaatcgcctctatcggctgggccatcgtgtccaagaaaggcaatgtgaaggccggcagccggatcttccctgatgccaagtctggcagagagagcaaccagtctagaagggccgccagactgatgcggagaggctacagacggaaggccaagcggagagccgatacactggccatcatccgctctattcaccccggcttcgatcccgaaggacaccccgatattgagagagaggccctgatcaaggccatcattacccctggcgctcccacaccttctctggatcaactggcctgcgccttccagagattcgccaagtctagatggccccagtacagcagaaccctgcctaagagaaccgaaagagaggaccagttcatccaagtgtgggttatcgccgagcggacataccccgacagattcacccctgatgtggccaccagactgctgcaggccatcttctttcagcggcccatcaaggacggcgacagagccaagtgccagctgtttagacaccacggcgataaggcccctctcgtcggatggacacacgagcctgagctgcagagatttgccatcctgagcgacctgtccaacctgaccatcggcatcggcagcaccgacaatctgctgtgcgagtaccctgacatcatcgaggacctggaaacccggtgcttcgagacaggcatgagctggcgggaaattgccgagcacgtgaaagaagtgatcggcaaaggcgtggtgttccggggcatcgacggccagaaaaaagtgggcagaaacggcatcggacccgccaagctggaaaccatcgacgaggaaggcaacagcaccaagagcaccgccagcatgtctgtggaagccgccgtgatgatctaccaccagatgaaggccgacagatgcagagccgccaccgccaagaaaaccctgattgatgctggcgccctgagcgcccctctgaccgccaaagatattaagcggggcgatagaaccctgaccattaccgagctgatggacatggccggcagaatcacagaccccaccatcagagccatctatcaccaggtggaaatgctggtcaacgagctgatcgccagattcggcaagcccgagagaatcgtgatcgaggcccagaaagagatcggccggtctatcgaggacatcgagaaagccatggccagagagcgcgagaagcacatcgagagacagagagagaacagagcccggaacgccgccatgggaacaaaggccagatttgctagactgtgcgccatcagaggcgatcggtgcttcatcagcgataggcctgctgccgaagtgggacacctgatcgccgatagcatcggaggcacactggaaatggccaatctgatccccatcgatcccgccatcaacaaagagatgggcaacagaaccccttacgaggccttcagaaagaccgagtattggagcatcatccagcggaaactgcaggccctggaagatgaagtgaaggccctgaagccacctaagggcaccaagggaacagcctggaccatctaccacagggccaagcaccagttcgatttcttcgcctggcggttccagagcaacgccagagagacacaccagcggaacttcagacctggcagcctggatgacctgcggtggatcgagaacctgctgttcctgggagtcgcccctatctgcgacaacatcagaatcgtgtccggcagaaccaccgagcggatcagacgggaaatcctcggcatggacaaggacagacgggaccacagacaccatgctctggatgccctggccattatgctggccaatcctctgaagccctgggacctgaagtccagcaactctctgggcatccctctgggaagaatcaagcaggcttttgccgacgctgtggtgtcccagaagcaggaccactctctgagaacagccctgcacaaagagaacgctatccccaagaccaagaggggagccgcctacagaaagatcggaacaggcgccagcgaaagagtggtggacacacagtctaaggcctactgtgaagtgtgggccctgccaaacggaaagtgggaagctgtggtggtgtccagcttcgacgccgctcagaagaactaccggcagggcatcgatcacaggcctcatcctgctgctcggctggtcatgagactgttcaagagcgatctgctcggaatcggcggcaagatctaccgggtgcaagagctgctcggatctggcagcatctacctggtggatcacagattcgccggcaccatcagggatgctagagccgtgtgtaaaaccggcgtgaacgtggacttcttcagcaaaggcggcgatagcctgagaaaagccggcgcaagactggtgtctatcagaaagagctgggtcggaagctgagtcacttttcgaattcggaaacttttccgaccgacaagtactcgatcatctagttggatcaggacgtcatgcttcgaattcgaaaaccattcgatgccgatgcctacaccgcaaagagttggatcaggacgtcatgcttcgaattcgaaaaccttcgtttcaataacc

SEQ ID NO: 37 - DNA, 207 bp, Artificial Sequence
  Cas 9t single guide scaffold
gttggatcaggacgtcattctaaaattgcaaaaaatgatgtcctgatccaataaggggcgcagcccccgcaccatttttccccgatctacggatcggatcggcgctgccttcgaaaagccgacatggaaaacccccagtttatcggggacgatgtaaacctgagtcgccacggcgctcatctcgcccgctctacggaccgggcttcg

SEQ ID NO: 38 - DNA, 21 bp, Artificial Sequence
  Synthetic Cas9t processed DR
gttggatcaggacgtcattct

SEQ ID NO: 39 - DNA, 182 bp, Artificial Sequence
  Cas9t tracr
ttgcaaaaaatgatgtcctgatccaataaggggcgcagcccccgcaccatttttccccgatctacggatcggatcggcgctgccttcgaaaagccgacatggaaaacccccagtttatcggggacgatgtaaacctgagtcgccacggcgctcatctcgcccgctctacggaccgggcttcg

SEQ ID NO: 40 - DNA, 22244 bp, Deltaproteobacteria bacterium GWF2_42_12
attgggtgggatagcatggatttgtaatccttttccctcagctcagcgaattttttatatcttggatcctttgatacatcttttacatagattggctttccttcttttgccgctgatcctgtaactccttctcccaatttgagccttaccttgccaatcgcctcttttacaaagcttgtggttgccgttaagacaagttcttttccattccagaggtatattgaggaaacatctacgcccaatctctttgcggtcttttctacgactgactggagcacctcatcaagcgcataatcagagcttgcaagcatgcttatctcttcaagggtcataatctccattttttccctgttcgccctttccatctgaaagacaatgccgccttcttcactaatctttacatgagaaaaagcatcgcttggataatatggattgatagccttgcttgctgacggcattgtcttatattcccaaaaaccgcatgcgcacacaagctttaccctgtaatcgtcaaccctctcgcttttcatcttctttccgcatttcctgcatcgtgtaataaattccattcagcgcctcgttaaaatcagttggaatttaacaggctgttgaaaaagccatcaacagccttgattacacagattagagaaaaagattacacagatatttacagaaatttcaaggagttttaatctgtgtaatcttgtcttttatctgtgtaatcccgaatagtcgggatgtaatcggagattgttgagtttttcaacaagctcttaatattcgggagtcggttcttcagtctataacctgaggtatttctttgttctcttccgaaatattaggcaagacgagttttacgaccttgccttcgtcaccaagtctgccaagtggattgccattcagcgtcacattgacaccgcctgcattccctatcagtatagaaaatttttcttttgctatccatttaaccttttcgccttctctgagagatacctcaaacggatcctgactatccacttcagctcgaatccatgtgcttttcgtggcttgtataatcaaggccatgggttcttttaagtctgccttagatgacacagattcttttgtatttttatcccctttttgtaaagccgaagggctaacaagtatgcctctatcgtcttcctgtttagaccgggcctcgtaagctatagcttccttcttgttgctatcagatacatcgttgccagtcgttagctgtatttgtgaataatcaggagcatctggtttatacttttttaagattacatatgctccagctgaaattattaaaaggatagtgacagctatagcaataagcgattgcgtagataatgtggttagtctctctaacttttcgtcttttgctttggcagcctcaacaggttcgaccttactttcagtaagggttttcatatacacttcataacgaagcagcgcatcatgtccgtctacaccaagatgtttgcaataagcctgtataaaaccttttacaaaaggcggtgcgggtaatttgctaaaatcgtctttttctaatgcattaagcagcccagcccttattttagttattttagccacatcttgtaatgagatacctctaatttctctttctcttttaagatattcgcctatgctctccattttgctaactcctaattatttatgttgaaggcttttgcctatttaatgtttattatttcaagaggtcaatatatcccttagccgaccttgccatctcgctgtcggggacaagtttaataacttcttggaatgcattcgccgcgcctttcttgtctttaatctttatgagaacaagaccgaggttgtaatgcgcatccgcataatttggcgcgttcttaattacgagtctgaaagtatcagctgcctctttatctttattcattctcatataggtaaggccaaggctattatatgcctgcaaatacattgaattgtttttaattgcctttttaaaactctcttctgcctctacataatctgttttttcgtaaaatgctcgtcccatattaaaataggcaagctctggagttgtataaaggatgtttgcgagcgctttttgggccgctgcgatagcagcatcccactcttttttttcaatgtagacagctgagaggtttgtgctggcatcagaaaattttgggtctattcggatggcctttttaaagtgctttatcgcatcatcatggaggcctttcgcaaaataagcaagccccagggcattatgatacgatgcctcatcaggatatttttcaacagcagttatcagttctttcagggcttcagggatatttctttcattgagatgaacaaccccaagcttataatgtatatctgcatcttcctttctatttgcctttgagcctgcacatccgtaaaagacagttgccaggaggcagtaagcggtaagcagtaagcagtgggtaataaacagtaactgcctactgtatactgcgtactgcatactttctttgactgctggtttctgacttccagtttctaaattcttaatcatctcatactccttagcctcttgtcggttttcaagatatatcttcaaaatgggttaattgcttaaattttctatatctatcgtaaatctcttttagtgtcagggtgcgcattctgttgaggctgaaatcctctacattaaatgaggccatgacactcccaaagataattgcctgtcttatattagattcatttgtgttgtcaatatttgcaagatatcctatcagtcctccagcaaaactgtcgccagcgccggttgggtcaaatacagattccaggggataggcaggtgctgaaaatatagaaccaccattgaacatcaatgcgccgtattcccctctttttattattactgttttagggccatatgcaagaattttttttgctgcctttacaaggttgggttcttttgcaaactctcgcgcctcaccttcattcaggacaaacaggtcgactttcgacaagagccccttaagcgcatcaggcttgccttctatccagaaattcattgtatcacatgcaacgaattttggctttttgacttgttcgagaacattcaattgaatctctgggtcaatatttgcaagaaacacatagggtgattttgtatagaccgctggaatatgaggtttaaattttttaaaaacatttaggtgagttgcaatggtatgggcttcgtttaaatcatactcatatcgcccctgccagcgaaatgtcttgccagctattttttgtaagccgtttgtatctacccctttttcttttaaaaaattaatatgctcatctggaaaatcttcacctataacagctacgaggctgacatctgtaaaataactggcagccgtagaaaaataagtggctgaaccgccaaggacattgtctgcctttccaaaaggtgtttcaactgaatcaagtgcaacagaacctacaaccaggatgcccatcaaaatcctcctgaatttcaagtaggcagtatgcagtaaggagtaagcagaaagaaacctgcctactgcctaccgcttactgcttactgttttttaccattattcatccacactgtcaattaatgtggaaagggcaactatctcaacttccctcttgagcattggtaccatctcttttatagctaaaatggtggtatggtgtggatggcctatagcaatggcagagcctctcttttttgcaacttctatcaattctcttatctgtccctttatgtaatctatatcctctttgttatctaaaaatacctgcctgctcaatgttttaagtccgagctcttttgctaatctataggcagttgatttattcgtggttttgctatccagaaagaatagatttttttcttttgcgatttccaatactatcttcataagtctttcattctctgtaaatttcgagcccatatgattgtttattccaataatatgcgggattgcgtcaatatcctttttaacctgtgaagcaacctgtttttcagacatggttgtaagtattgccccttcccctggatcgttgttgcttaaatcttttggctccatggggagatgcagcaacacctctcgcccgtttaaataagcctcctttgcagcgttctctgaattcggcaaaaatggcaatacagcgatagcaatgggggcatctatttctaatatctctttaagctgtgccctgtcgcggccaagatcgtctataattattgctaccttagccagtggtatatgcggcttttggggaacaggaggttgaggtttctctatctttgctacaactgtagatgttgaaataggttctggaggtttttcttttgtactgtagctgaggtatagaataaaccctaatatagcaatgataaaaaataccgttgcaattcccaaaacaaaaagggtctttgatgagaaagacccttttttgctccagttatttttagccattttttaacctatatagctaatccctcttagatgagccactcttatgattccttctttattgtctctttaaatatataccaactcttcagataatccaatgcccttttgagctgcatgtccgcttctgtctcttccttttcttttactaccttttcctctatctttatctttttttctttaggagattcaacctcttcggcttcaaggtgtctttctagttccttttctttaatatgttcttttattgtttctccaaccacaatatcgggttctatgccttttgcctgtatagatcttcctaaaggcgtataatatttagaggttgtaagtctaaccgctgaaccatcgcctaaaggaatgatagtttgaacagaacccttgccaaatgtctgggttccaagcacaaccgcccttttatggtcctggagcgcgcctgcaacaatttctgatgcgcttgcgcttccaccatttaccaggactataatcgtatattgggggtgcgttccatctgcccgtgcctcaaacttcatatcctggcctgggcttctgcccttggtatatacgattaaacctgattccaaaaacgtgtcagctactcctacagcctgctgcaaaagtccaccaggattattccttaagtcaaggatgagaccttttaacttgctgtcccttgagccaagcttatttagcgcctcttcaagttcattagtagtcttttcctggaaatgtgcgattcggacataaccaaacccttcttctaacgttttatattttacactcttaatagctatgatatcccttacaagtgtaaattcctgtggttctttaaagtcctcccgcattatccagatagtaacctttgttcctttttggcctcgcattagctttacagcatcgtttatggtcatatcctttgtcggcttatcacctatttttactattttatctccagcctttatcccagccctgaatgctggcgtatcttctatgggcgcaatgacagtaattatgccatcccttattcccatctctatgccaataccaccaaaggcgcctttggtttcaacctgcatttctttatactcatctggagccataaaagacgaatgcgggtcgagcccgagaagcatgccttttattgcgccataaatgaggtttttggggtcaacatcttctgcatagttatcctctacgatagagatcacgtcggtaaatatttttagattctcatatatctttggaggcacagcggaaaccctgtgattaatcccccatagaagaacgctcgttattagagctattcccaacagcgtcatgagcttacgcccttttaacctcttgaacataaatgtattcctcctgagtttatttaagataaagacttgatgcgatatgcgaaagggttaaaggaggtagcgctacctaattgccttctgccttaagcctgacgctctctgtcttatcatatgtcaaccaatttaccgggtctaatggtacacccttctgtcgtatctcaaaatacagatgaggtccttttattgaacctgtatctcctacaagtgcgattatatcgtctctttccacggtagaattaacctctgcgaggatttttgacaaacggccaaagagtgtatgatacccattgccgtgatctataatcaaaacctttccatagcccctgaaccatccagaatatacaacgtggcctttatatacggacttaacctcagcccccatcgcagcctcaacctcgatgccgttattaaaggtaacagtgtgaaatttgggatgctctacctttccatacattgaaatgactttgccagctacaggcattggcaaatgaccgcgcatagacgcaaaaccgttcgtctcattatccaaaggattattaaccgtctcattcttcaacctatctatgaatagttgaagctctctgcctgcgctttccatttcctcaattgcggcaatctgtatttcctttttcttttttatatcatttaaaaggacctttttcttgtctttttcttcttttatttcttcctttttcttttcaactgcatcttttaaagagcctatctcctcctgaaactctttgagccgtattttctctgcttctaagagcagttgattctcattgtaatcttttaacagggttgtatcatgatctataaccttgtttacatatttatatcttctactaaactcattggcaaaatttggggcaaaaatgctctgggttatgcccgccctattcatcttgtacattgcaacaagcctttgttttagaagttccatcaaccttattctctcctgaatcaactcttttattcggatatccacatcagagtttttttgctttatgacaacaaggctgttttctatcttttctaattcctcctcctttttagataaggccttgtcaagcctttcaagtccttcaagaatagcatattctttttttacattatactctaaacttctctttttttctacaatctcccttttgagggattccagttgtttttccttttttacaatctccttctttagcttggcaggttcagccgcataaccatgaatggccggggaaacgagtaaacacaaaacaagatagaaggggtaggttctaagggtcaataacaataataggtttaaattatggattgccacagttttaactttctctttttaagcagtcattggttttttatagaatttaatttatcctatctaaaacaattaaatttatactctttattctatctttcagcgcccccatttttgaatctttgccaagctcgaggtttgtaacaacagctttatttacagttctgaaaccatctttatcataatagccaaaaagctgatggagcgcatagtagtttccatctacgttgccgagatagagcatgatatgaccgttgagggctaataaggtaatgcctgggattgcggaacttattgcgttatcaatatctccgtttgccggcatttcatcaaaacctgctaacagcgtcccgacattagattgtttgccagaatgcctgggaaggtttatgcccatcgtcgcaaacacatccttgataaagcttgaacagtccctcaaaccgtttctacctccccagctgtatttctcgccaaggattttaaatgcctgttttatcacatttttcttggtatagggaagatagccaatattcacatcgctgcctttcttaataaatgcatctgcccattgaagctgaccattgcccattttttttggaaatttaataatccaatgtcttttatcttcaccgctaagcgcaaaggaacgacccatgggaatggactcaagaacctttgcacccttattgtctgcaaaaactgccactcggtttcctgttattaaaacgaatttttcagcatctattattaaatcgtctttgctatttacaaaagcaatgatattgagttttatccagccacgtacaatgtttgtctgaaagaaaccccactgtccatccctactcttgtgcaggagcgccaccacctgaggaggatatatggcggaatactgaaatgcatcaaaatctttggcagaagggcctttaaacacaggttcatcagtagggaatgaccttatatccgtccttttagttacaatcccaaatgtaagttcattcgattcatttatagcctcaaaattcatattgtctaatagttcttcataaaaggcgtcttttacaggttttcccttcttatcaaacatatttttatctactgggagagggtcttcaagcagccatttttcaagctcttcttttgataccgatgttcccatttttattacatctgccatttcattgttttcgccaagaatcaaagcgtttaattctgcaattttctctgaagaaagtattacctcatcaggattatcaagccttccaatccagaagtctgctttaaatattggaaggtaggcgcttgcttcgagcaatacgggagagagaaaaattatactgatgactatcttcaaaactttcatgccaactttgataacataatacgtcaatattctcaagtcataaattatgttcggtaaaatcaatgtctatttttgggtatgtttcaagctctggacaataaaatactactgtgatatacggagtctgtggaaaagattagaaatcctattccaacagttgatataataattgaattaaaaatcccccctcacccccctttttcaaaggggggaaggggggattgggggattgtccttatagagaggaaaaatcccccgtttggctgggcaattccaggcggttttgtggattatggagaaaccttggaagatgcggctatacgagaagctcatgaagagacagccttgaaggtgagactaaaatgccagttacattcttattcaaaccctacgagggatccccgatttcatacaatttccacagtatttgttgcagaggcagatggaacaccgcaagccagagatgacgcaaaaggaatcggcgttttcacagaagacaatctgcctaagcctctggcctttgaccataagaagatattggaagattatttcagatggaagaaggaagggtggagggtgtttgaggaaaaggcattgcagtcaaatgatggctgtgataacttgcttagctgaaaggtaattggattatgataaaagatacccaactattaaaaaaatttgaagacacaatcatgaaaaaagagggaaggctttcttttagttactctatgcgcatatttgaatctctctggaatgaagggataaaactcggaatattgcctcccaagaaaccattagaaggaatagaagttgatataaagatagcacaggtgctcaattcatgtttgaagaaatcctcgcaaggataggcgcagcgcttgttaagtgcaatattccctacatgattataggcggacaggctgtattgctgtatggagaaccgcggctcacaagagatattgatataaccctcggcgttgatacaaatcaccttaatcaacttcttgcaatccttcaaaaaataccactcaagcccataccagaagacatagaatcattcgtcaagcagactatggtcttgccggcgctggatgaaactacaggcatacgggttgactttatattctcatttacgccttatgaaacagaggcgataaagagggcaaagagtataaatattctcaatcaggaagtatgctttgcctctgtagaagatgtcataatacacaaaatatttgcaggacggccgagggatttagaggatgcaaggacaatcctcttaaaaaatcctgatattgacattagatatatacagggttggttaaaggagtttgacgcatcaattggcaataaggattttctgaaaacctttgaggacatattaaaaaccataaatcagtagaataggcttgtttttagtggtaaaaggaaggacagaaggtttttgagatatggtgatccccatcgtcataatcaaaggtaaataaaaatggaaagagaacttgttttaggcattgattacggcggcaaatataccggtcttgcagttgttgaccgaaggcataatcaggtattatatgcaaacagactgaagatgcgggacgatgtcgctggtattctcaaagatcgcagaaagcaaagaggtatcagaagaaccgctcaaaccaagaaaaagcggctgcgtgaattaaaaaactatctaaaaagtattggatataacgaatccacagcaacatttgaaacggtttacagccttgcccataaaagaggatatgattatgctgatatgcctgaagaaaagacttccgaagaaatagaggcaatggatgtggaagagcgcaaacagtgggaaaaagagaaacaggaatgggaagagacaaaaagaaattcaaggcacaggaaagaagtcgtaaaagatgtccataaagcaatgattgaaggaagagcaaccgaggagcagataaaaagggttgaaaggatttttaataaacagtaccgacctaaaagattcaataataggattttaactaaatgtaaggtagaagattgcggagttaatacgccgctcagaaagaatgtgagagatttgcttatagaaaatatagtcagatttttccctatagaacaatctgaaaaggataatctcaaagatgctgtgcttgataaaaacaggagggaagaagttaaatcatttttcagaaaacataagactgatgaacatattaggaagcaggtttatgatatagcggataacaagctttcgggtagaacggtcttttgcaaagaacatatccttgaacatactgagcatagcaaagaggaacgaaaggttttccgtcttgcgccatccttaaaaacaaaaatagagaatgtccttgctgttattaaagacgagatattgcccaaatttactgtcaataaggtggtaatggagagcaacaattttgatatagcagccaaaacacaggggaaaaaacgattggcaaaagaagaatatggtaaagggcccagggaaggcaaagagaccagaaaggaagcccttcttcgagaaacggatggtagatgtatctactgtggaaaatctattgacatatccaatgcgcatgatgaccatatatttccccgaaaggccgggggcttaaatatctttgcaaatttagtagcatgttgcgctgtatgtaatgagaataaaaaagggagaactccgttagaaagtggaatctcgcctaaacccgaaataatagccttcatgaagaatgatttaaagaaaaagattcttgaggacgcacgaaatatcaatacagtagattttaataaatacatgtcccatgcctctatcggctggagatatatgagggacagactaagagagtctgccggcaataaaaagttgcccatagagaggcagagcggcatatacactgcatatttcaggaggtggtggggatttaaaaaggaaagaggcaacacccttcatcatgcccttgatgctgtaatccttgcttcaaggaaagggtatagtgatgatggattggtggatatgacccttaagccaaaatataataaaggtggagggtttgactctgaaaaacaccttcctgaaccgatagaatttaaaagggataaaggcagcaagggctctgcattgcatgatagaaacccactgtcttataagaagggcattattaccagaagatttatggttacagagattgagtgcggtaaagaagacgatgttatttccgagacatatcgtgaaaaactaaaagaggcttttaaacgatttgatacgaaaaaaggaaaatgtttaacagacaaggaggcaaaggaagcaggtttttgcattaagaagaatgaattggttatgagccttaaatgcagcattaaaggaaccgggccagggcagatgataagaatcaataataatgtttttaagacgaatgtccataatgttggcgtggatgtttatcttgatgaaaaaggtaagaaaaaagcatatgagcgaaagaatccgagactgtcaaaacacttcatagaaccaccaccgcagcctaatgggagagtttcatttaccttaaaacgaagagatatggttacagttgaaggtgaagacgcaatctatagaataaaaaaactcggaacatctcctacgatagaagcagttgttggtagtgatggaaaaacaagaactgtctctgccaccaagctgacaaaagcaaacagtgctgaatagtttttaaaattaaatgtcctaacccacgatgaaggcgccatttctggcatagaggctcaggctggacaaacccaagcgtatatcccttcactgtataggcgttttacaattaaggctcttttaaaatttcgcaaaccccttaaggtttgtttcaaaaaatggtcttgcggaaattttataaattgtttttgaaaatccctttgaaattccagcaaaacaatagctgtttcacgccatattttcaaagagccttaactgtaacttcgatcttctcgaaggagatatgcgtgggcagatgtttcacgccatattttcaaagagccttaactgtaactgtgtgaagcggtgcaaggcgcatcccgcctttataagtttcacgccatattttcaaagagccttaactgtaactccttcttatacctgcggtaggccgcctgctcagaagtttcacgccatattttcaaagagccttaactgtaacgttcctcaatctgtgcggaaggccgccccgaaagttgtttcacgccatattttcaaagagccttaactgtaacgattgaaaatggcaaccatgagctcggatttgtgtatgtttcacgccatattttcaaagagccttaactgtaacttcataataccacactgaaacgaaactcatgctaatgatacttcggctgtcttgcctgtagattcattaataacaaggagtttaccctcatcctcttttttcaactgttcattggcattcagcaattctatgccatagaccgttccatcaggcgccaaatctatgttcagttcatcgctgattttcacagtctccacttgtgctggtttttcatgcaggcgaatataagctatgttgtatctcggatcatatgttaacttcatatatgttccaccgtaaggcttgcaatatggcgtatactgctttatagttacaccacataacctcaaaaatcaagaaaatattgaggaggtctatatggataatgaaacatcaaaaatcctccgcatccatccggaatatcaaccggaggaaaaactcccgccagcacagttcaatctctttacaggagaagcagagccagtcccgcaactgattgacaaaaatattgatgatgaaatatttgttgacgatgggatttcgcttgtgcagagcggagattcgtcgcaaatagttctgtcaggttttggcatcttcttaagtaaaaagagtgaacgccttattgtaaaaaagagcagtaaggttatttatgaatttcccttttttagactgaatgaggttattgtcgcctctcgtgggatgtcattatcctctgaccttgttgaagagttatgccaaagggggataagactgaattttttgacaaatggcggcaaaccgtatgcaatgctttcctcgcccatgctctccgcaacggttgtatccagaagagagcagattatggcgtttaatgatgaaagggggcttgaattctcaaaggcggttgtagaaggaaagattgtaaatcaggaaaggcttataagatattttggcaagtatattaagaccgtagatattgacagatttaataaacttgaaggaattgctgatgatattaaaaagatgagcggacaggtagataatattgatgggcaaaatattgaagatgcaagaggtggtttgatggcaatagagggtatatcaggcaggttatattggggcgcggtcaaagagattattgcaggaaaggttgagtttttcggccgtgagacaagaggggcagtggataaggtaaactctctcttaaattatggttatggcattctctattcgcatgtctggggagcaattataaatgcaggtcttgagccatttgcgggatttctgcatgtggacaggcccggcaagccgtctcttgttctggatctgattgaagaattccgccagcctgttgttgataaggttgtaattgcccatatcaacctcggagaatccattaacatgaaaaatggattgattgaagaggaaacaagaaagacaatgggaagcaaaattatagaaaggctggaaagccaggaaacatttgaaggcaagaaatataaaatcaggtctattatccagatgcaggcaagaaacctatcggcttttttaagcggcaggagagaatataaggcgttcaggtttaagtggtaattagttcacgctgaaaaatgcccatctgctgcgttgccttcgtcgctgcgctgctcacatacctaaaagcgtatgctccgctgctcgctcctcggcgccttgcatctgtgcaatttttgagcgtgaactatataaaagcatttttcaactaattatgggagaacatactgtttatattttctatgacatagaagacgacggcatccgcaataaggtggcagagacctgcaaagactatgggcttcaaaggattcaatttagcggattttcaggcacgttgggcaaaaataagcgcgaagaactttttttaaagctttcctttctcattggcgataaagccggaaaactccttatgctccctgtttgtgaaaaagacatcaatgccaaaagggaactcatacaggaaaatagagatgattcatctcagggttagcgaccttaaacagtttgtctattgtcaaagggttgtgttctataactatgttatgcctgttgagaaaaaggcaacctttaagatggaatatggaaagatagcagaggataagatagacagattagaaaacagaaggaaacttaaggaatacggcttatccagcggcacgagagaatttcataagcaattatattctgagagatatgctttatccggcaaggtggatttgctaattaaaaccaaagattcttattatcctgtggattttaaatatacaacctctcagccgcacaaaaatcatctttatcagcttttgggttatgctataattttggaagatgtatataatcgcaatgttgataagggttttgtatacttaattccaaaagaggatgctgtggtttttgatttaaccggcaaagtcaaagaggaaacaaaaaatttattagacgatatccgaaaaatgatacactgccagcaaattccgcctccggtaaattctccggccaaatgtcttgactgcgaatatcgtaatttttgcggagatgtattataatccgaaatagtattatgcccctttcaaaagaagataaattgctcctcttaaaaatagtaagggagacgattgaatcttatattaagactaaaaagattcctcctttcaatgtgagcagcccggcgcttctggaacatcgtggggcatttgtttctataaagacaaagggcaatttgagggggtgtataggcatctttgcgtcagaaaaacccctttatctcactgttgtagacatggctgttgccactgctacgcaggaccccagatttattccgcttactgcaactgaactatcactgattaccatagagatatcttgtcttacggcgctaaaaaaggtaaaagatactactgaaatagaggttgggagggatggtctgtatattgtaaaggggtattgcagaggcgttcttctcccgcaggtcgcagtagagtgttgctgggacaaagaaacatttttagaacatacttgccttaaggcaggtcttccatctgacggctggaaggatggggctgatatatatacatttgaggcagaggtgtttggcgaaggcgattgataaggccgtaaaatagcgattaaaaacttcgctattttacaaacctgcgatttgcctgctccccgatagagacactcggggacaggcatacttcgttatcgacacgctcagcgtgccttgaccaagcttagcttggtcggcgcatttttgctcctcaccgtatcctccccctgtccccctgtggggacagatttcaaatctgtccccagaaaagggggagggctcgtcgcaaaaacatgtccctgagtgcttttatcagggatggcctctgcctcgtctgacaacccccgatagaatcaatcgggggcggtttccccaaaaatcaaacgctatttttgggtaaggtatttttagtgaaaaatattcttactttatgctaaaataaaaccccaaatttgtatacgaatgttaaaaaataattctacaacaatccatgagactgattaaatctatacttattattttaattatctgtttaataacttccatttttaggacaaatgcagaggctataccagcctttgcaagaaagtataagacgagctgcacaacatgtcatgcagcttttccaaaacttaatgcctttggtgaggcgttcagaagaaacggataccagatcccagaggttgacgaacgctatgttaaggagaaacctgtcagcttgggcgctccggcgtggaaagaggtttggcctgaaggaatctggccaggcgagattcctggggcaatacccctttctttatttggaagtttgttttaccgatatgatgtaaactctcgggtaaagcatgattttacctttccaagtaccatagcgcttgaatcagctggaacactcggtgaagatgtatctttttttggcagtgtttctcttattagaagtggaaatgagttcggcggtttgtggcgttttttcctgaagtttaatgatacattcagcagctcgcttcctgaccgccttttgaatgtgactatcggtcaattcgaaccgtcagcagtcccggtctccaataatcgccgtttaacatatgcgccatatctcattaatacgtttacagtcgggaaaaacagctttaacttctcaaatcagcgaggcatagagatagatggcattatagagagccgcttagaatatgcagtgggcatagttaatggcaacggtacagggaagacagattccacaaccaactctgcagacaataatacgacaaaggacgcctgtcttaaggttggatataagttcggcggcattggcctggatggttctggccttatctcagacaaagaagatgtcctgccagaggtaaaggatgaaaaatcgctatatgttggcgtaataggttattcaggcaaaaataaagtggttgtttcaaacagcacgtatgacgataatttctataggtatggctttacctttgacttcaattataaaatttttaatctgtttggcgcggttattcagggggagcatagtaatccgagtggcaattttagtgatacctctgtaagttcctatttttctgaagccgaccttgccatttatccttggcttattgcagcggtgaggtatggagtagctgatatagaacatgagaaggaaaataaagatgaagtggttgtaagcttgttggcgctggttcgcgcaaatataaagcttactatcgaaggggcgctgcataccacaggtggcgagggggacctgggcatagttaaacttgattttgtcttttaagccaacattttcttatgccaattcgctctgcgagaatgccgtgtaaaccccgttagagaactatgtttcctaacgaccaaagatagtggcagcgcccaccgtaagacctctggtctcttacggggtgaactaccgtctctaaggaatgatttatactaccgtccgcctcaggcggaggagtcggaggctttctctaacggggtaaaatatacttgtagcgccatacaatgacaacacctcaatttttcaataatactttctcatagacctgggggaagcatctatgattttaatagacataagcaagaaactgtttaactcaaaaaatgcagttgcattttttgacaggagtggtaagtatgcgattgtgccgttgcagatgaaagcgcttctgcggttgagatatttagccgcatgcgcaatagtgcttattttgagctgcatatcgcatgccgagactattagaggaaccgtcaagctaaagagtgaagatgatgcggggaaagtagttatttatgtcgaaaaagctccagggggatttataccgccagagaagcatccggttatggaccagattgaactgatatttgttccgtatgttcttcctattctcataggaactactgttgattttcataatagcgatgatgtgctcaataatatctttactccgtcatgggcagggcacaaatttaatctcggcacctatcccaagggagtagtgagatcatttacatttgaccgtctcggagaggtagcattattatgcaacatccatcctgatatggagggctatatactggttttgcagaatccgtattttgctatccctgacaagcaggggaggtatgagataaaggatgcgcctccgggggtttatgatttcaaaatgtggtataagaggaaagtaagcccttcttatacggtgactgttgaaaaggggaaagatgctatagtggattttaaataagggtaacgggaggtaacgacgttgaaggtgacaataagggcaaaattaatgattgggtatatagcagtcctaatctttatgattatggtgagtatcttcgcatatatcacacttgttaatgtcaacaaaacagtaaatcaaatacttgtttctgcccataaatatgacacggttaacagcttgcggcagtctgttaaacaatttgttgatgtcagtgattcgttagtaagagggcagattcaggatgtgggttattatcgctctctaacgcttgatgtgaaaaaaaagcttttgtatgtccgtaaactgcgactcaaggagcatgaaaaggatttcttggaaaaagtagatgctgaatttgactctatacaaaatctaacagagcaatccttaaagtggacagcagctttgcggaacacaaatctcgttggcgtgctcaaggaattggacaaggccaagccagttataatcagcagtgttgatggactgtatgatgaggcatggcgctcactggataacgttactattatgactaacgataatatgaggaagggcctgcaacagattctgacattttcgataatcgctattttggcgggaataggaatatctatttatatttctcaaaagattactacgccaataagggcattatccatgtccgctgccagtgttgcgaaaggcgatttggataaaatggtggagaagtcgtctgcagatgaaatcggggaattagttgcttcgttcaatcagatgctggttgaattaaaaagctcaagggaacagattgaaaagtataacaaagagcttgagaccatggtggatgaaaggacagcagagctggaaaagacaaaggaataccttgaaaatattcttgaacattcgggagatatgattatcacaacaaccctctatgatgagatagtgcagtttaacagaggggctgaaaacatactgggctatgtcaaggaagaccttggcggaatcaagataggagatatttttgtcaataagagtgagtataagcggataagaaagaaggtaatcgaggacggagagatatccggttatgatgcgcagcttatgaagaaaagcggtgatatcatacgcgtaagtcttacgctctcgcgtcttgaggacaggactggcaatattatcggtcttgtaggcataggcaggaatataggcgaggataaaaaaagtatttaaatgaagatacataaaaaggggattcaacgcaggatagccatttttatatttgtcgtagggatgtttcctgtcatgataagcatacttttagtttatctgcagggggtgagggagcttagagtaagtgcgggtgaaaattttgcgcgagtagctaaagagatagcgagtaacgccgaaattgttatggagcaatctgttcacgatgttaaaagccttgctatttcaccggtcttaagaaatgcagcgatattagcaaatgaatcctatacaggtaaagataaggggtcaatagagaggcatatccagaaaattgatatgcagtggttgcagcatggagacaaggaaaaacgttttggggcgtatgtatccagtcgggctgcgcaatacttagcagaaataaagaaaaatacagagggatacgttgagctttttataaccgacacaaaaggggtattagttgcctccgcaggaaagacaaagtatttatactatggcagtgagaagtggtggcagacagcctataataaaggtaaaggagatatttacataagcgagatataccttgaccctgatatcaatcagttcctacaaagtgttgcagtgccaattatgtctgagaaagaaggaaaggtgctgggagttattcgtgcagttagtaccatagataagatttcgaagatagtgcagaaattcagaattggaaaaacaggccatgcaatgcttataaattccgttgggattatcctcctgtgtcctatattcccgcctcaggttcacagggttactgacgagctgatgaataatattaccacagcgccgttacttggctgggcggttgttaaggataatgcccatgaaggcattaatgctgttgcgggatttgcccctattgcaacgaccgatattgtaaagaatgcctttgatggaaacaaatggtatatctttgtcagtcaatcaccggatgaaagttatgcccctatttatgttttgctaaagagggttttttttctactgatattctctattttaatactctctgctatgggattctgggcggcaagaaagattgtgaagccgatagagacgcttcggcaaggggttgagataattggggaaggcaatttaaaccaccgtatccatattaagacacatgatgagatagaggagcttgcaaataaattcaatcagatggtggaaaaaattaacaagacacattctgaacttgaacagcgtatttcggagaggacagggtttttgagaaaaggctatcaagagatggaagtaatgagccgtcttaagtcagaatttctcacaaatgtatctcacgagctgaggacgccgcttaattccattttgggattttcagagctgctccacgataaggtttgtggagatctgaatgaaaagcaatctgaatatatagggtatatttatcggagcggtaaacatcttctcgatataataaatgacatccttgacctgtcaaagattgaggcaggaaagatggagataaaaccagaagattttttagtttccaatggaataaaagaggcatgcgctatagtgactcctttggctataaagaagaaaatacctattgatgttgaaatagcacctgatgtttatactatcgtggctgatataaggatgtttaagcagataatgtataatcttatcagcaatgctgtgaaatttacccaggaaggcggccatgtcagcattaaggttaagtccaacaactattatcttcaggtttcggtgattgatactggtgtaggtatcaaaagggaagatatgggcagcatattcaaagaatttaaacaggtctcaaccggagatcaggagggaactggtcttggtcttgtccttgtaaaaaggtatgtagaaatgcagggcggtagtatcagggttgagagcgaatttggcaaggggagtaacttcaccttcaggcttccgatgaatattacgaaaatataaagagcaggctatgggcaatgggctataggctatggggtttttcctattgcctatcgcctctaccctactgcctacttttaataggcatataaatttccatatggtcggagggaggcaggtgtcagcttcttaaaaccggatttcattatatcttcggttttaaggttaaagtctttgcaataatcctctaaatgttccttgagaaattttttgtcctcttcgtttaaatctgtaattccaacctcagcgccgcacccttttttatccacatcgcctcttataaatatcacgccgccatgcatgcctgtgccaaggtaatccccaaccaacggccttttattgtcgccatcaaggcccaggaggattaatacgccgcctgccatg

SEQ ID NO: 41 - DNA, 4598 bp, Unknown
  Soil microbial community from Rifle, Colorado
  misc_feature (1)..(9): n is a, c, g, or t
nnnnnnnnngcttgcaatgaaatttaatgctacggatactaacacagaactattgaacattacaaaccttaaaccccttaagcccgggccgtacattttccggcaaattgcggccttgcacagcggatataaaacgaaaaaactttactgcctttcttgctttcaatcatggaagttgttataaagatggttgcgaagatggcaggaatgaaagaaggtagcaggcagggaagggtgtggtatgatggctgcaaatttgacaatttctaattttaatagaaccgttgtcccattaaccatcttgctaaaaaccgatgggagaggaaagagagtgtttgaggtttagcatgaggaggttttatggaaagagaaattgttttaggcattgattacggtggcaaatataccggtcttgcggttgttgaccgaaggcataatcaggtattatatgcaaacagagtgaagatgcgggacgatgtcgctgatattctcaaaggccgcagagagcaaagaggtatcagaagaaccgctcaaaccaagaagaaacgtttgagagaactcagaaactatctaaaaagtattggatataacgaatccacagcaacatttaaaacggtttacagccttgcccataagagaggatacgattatgctgatatgcctgaagaaaagacttctgaaaagatagaggcaatggatgaggaagaacggaaacagtgggaaaaagagaaacaggaatgggaagagacaaaaagaaattcaaggcacagggaagaagttttaaaagatgtccgtaaagcaatgactgaaggaagagcagccgaggagcagataaaaagggttgaaagtatttttaataaacagtacaggccaaagagattcaacaacagaattttgaccaagtgcaaggtctgcggaaaaaatacgccattaagaagaaaagtgagggagttgcttcttgaaaatatagttcgcttcctgccgttagaaagcgaaatgaaagagactcttaaacagacaattcttgaaggacagcaggaaaatatcaataaaatattcaggaaattgaaatttaatcagaaagactggccgggcaagaatcttacggacatagcaaagaacaaactgccgggcagactgcctttttgcaaggaacatttctccgagaatgagaagttcaccatgattgaaaaatcaactttccgtcttgcaccatccttaaaaacaaaaatagagaatgtccttgctgttattaaagacgagatattgcccaaatttactatcaataaggtggtaatggagagcaacaattttgatatagccgccaaaacacagggaaaaaagcgattggcaaaggaagaatatggcaaagggcccagggaaggcaaagagaccagaaaggaagcccttcttcgagaaacgaatggtagatgtatctactgtggaaagtctattgacatatccaatgcgcatgatgaccatatatttccccgaaagtccgggggcttaaatatctttgcaaatttagtagcatgttgcgctgtatgcaatgagaataaaaaagggagaactccgttagaaagtggaatcctgcctaaacccgacgttatacgttttatggaggatgaatttaacagacttaaaattaagatgtggaagatcaggcgaaagcagtcattgacaactgaagagattatggataaggcaaagcgcaataagcttgaactgaaactaaaaatccttgaagatgcgcagcaggtcaatgaactcgatttcaacaagtatatgtctcatgcctctatcggctggagatatatgagggacagactgagagagtctgccggcaataaaaagttgcctatagagaggcaaagcggcatatacactgcatatttcagaagatggtggggatttaaaaaggaaagaggcaacacccttcatcatgcccttgatgctgtaatccttgcttcaaggaaagggtatagtgatgatggattggtggatatgacccttaagccaaaatataacaaaggtggagagtttgaccctgaaaaacacattcctgaaccgatagaatttaaaagggataaaggcagcaagggctctgcattgcatgatagaaacccactgtcttataagaagggcattattaccagaagatttatggttacagagattgagtgcggtaaagaagacgatgttatttccgagacatatcgtgaaaagctaaaagaggcttttaaattatttgatacgaaaaaagggaaatgtttaacagacaaggaggcaaaggaagcaggtttttgcattaagaagcatgaattggttatgagccttaaatgcagcattaaaggaaccgggccagggcagatgataagaatcaataataatgtttttaagacgaatgtccataatgttggcgtggatgtttatcttgatgaaaaaggtaagaaaaaggcatatgagcgaaagaatccgagactgtcaaaacacttcatagaaccaccaccgcagcctaatgggagagtttcatttaccttaaaacgaagagatatggttacagttgaaggtgaagacgcaatctatagaataaaaaaactcggaacatctcctacaatagaagcagttgttggtagtgatggaaaaacaagaactgtctctgccaccaagctgacaaaagcaaacaatgctgaatagtttttaaaattaaatgtcctaacccacgatgaaggcgccatttctggcatagaggctcaggctggacaaacccaagcgtatatatcttctcgtataggcgttttacaattaaagctcttttgaatttcgcaaacctctatgggtttctttcaaaaaatatcttttcggaaattttgcgatttgcttctgaaaaaccctttgaaactccagcaaaacaaaagcggtttcacgccatattttcaaagagccttaactgtaacaaagtaagagtgccggttgatttgaaattcgtgccaaagtttcacgccatattttcaaagagccttaactgtaactatacatattatattcaatttcctgcaaccattctgtttcacgccatattttcaaagagccttaactgtaacctttgattgtctcaatcagggtatccaatttaagggtttcacgccatattttcaaagagccttaactgtaacaataagaagccgtcattgctgcaagcgtgccgtcgccgtttcacgccatattttcaaagagccttaactgtaacatgtattgcctgcaacattctggatgattccaaattcgtttcacgccatattttcaaagagccttaactgtaacttctttggttcgggtgcggtgttgtttgcaaggcgtttcacgccatattttcaaagagccttaactgtaacttcataataccatactaaaacgaaactcatgctaatgatacttcggctgtcttgcctgtagattcattaataacaaggagtttaccctcatcctcttttttcaactgttcattggcattcagcaattctatgccatagaccgttccatcaggcgccaaatctatgttcatttcatcgctgattttcacagtctcgacttgcgctggtttttcatgcaggcgaatataagctatgttgtatctcggatcatatgttaacttcatactatacccaaactctgccgcgcctctttccgccaatcgttcctatgcgtgtatatgaaattttaccatatacggcttctatcaatatgtcggaactgttgcaatcgcaagacttgcaatatgatttgcactgctttatagttacaccacataacctcaaaaatcaagaaaatattaaggagggcatatgggtaatgataattcaaaaatcctccgcattcatccggaataccaaccagcagaaaaactcccgcctgcacagttcaatctctttacaggagaagcagagccaattccacaactgattgacaaaagtattgatgacgaaatattcattgacaatgggatttcgcttgtgcagagcggagattcctcacaaatagttctgtcaggttttggcatctttttaagcaaaaagagcgaacagcttatcgtaaagaagagcgacaaggtcatttatgaattttccctttgacaaacttttttctacctgcaataattggccttgccccaaaaatagacattgatttttggggaaactgacaagttgcctgccagacgaggcggcggccatttttgtgacgcaaccgtatgctcaccgatacggtgaggaacaaaaattggccgataacgaagtatggcaggtaaatcgccagtttgtaaaatagcgggggttttaatcgctattttacggcttgtttttaaaattctattgtattgcaagcttatagatgatctcagaaaacaaaagactcaaaaacacacccctgcatcaaacccataaagaactcaatgcgaggctggttccttttgcaggatgggaaatgcctgtgcagtacatgggcgttatagaggaacatcttaatgtccgcaaggcctgcgggctttttgatgtaagccacatgggtgagatagagatatccggccctaaggcccttga

SEQ ID NO: 42 - DNA, 10629 bp, Nitrospirae bacterium RBG_13_39_12
  misc_feature (1)..(13): n is a, c, g, or t
nnnnnnnnnnnnnctggttaaaactacctcatgtttattataacaaatttgtttattttgtgttgaagtatccactgcacaggaagaaaaggggaggtgttaatgctcactaattaggtaactttttggagtttagcagttcatcatatttaccatcgagataagtgcttgaaaaatatggtaggcacgaggggtatcgaacccctgacctcttccgtgtcaaggagagtctgtcgtatgtcgaggttttacgaatctttataaacctttgtaaatttttgttgcaaaggatttggggggagaagggttttatatccttttcccttcttttttgactgtctactatacaaaaactatacaatggcattaaacaatacttatttttcagacccccctacatcccctttttcagaagaggggtgggggttaagtgcttaccccctttctcgcatatgaaatcagttttatgggaaaggctaaaattgacctaatagacgctgtctaatttaaaggttcactttgaggatttcacctacaactattcatcttcagcaagttcaaagagttaactatttccgagagaaattaacaactattcttttgtaaactcatcaataaatttatcaacattatgtgtcttacgctgtatcaacttaaataaacgccaaagttcccctggggctgataactcttcaaagtcaaattcatattccaagataaaatctcttgacgctaatgcaagctgtggacttgaacttccctccttaatataaattactatagatttgttaccaaattcacaataataaccatctttcagtgtcgagatatcatcacgaatgaactgataaataatatttggagataggtcattaataagactattccaatctaatttatttgcttttgttttttcaattaagctccgaagaaaggtcatttctttcataataaaaattcctcttttttagaaagtttgccaagcaaaatagctaccttatctgcgactgctattgtatcaatattgtctaaatcaccatctaataatcgttttagtttctccagttcccttttatgttctttcccaaattgaaattcttccaatgtgcttatgagtctttttaaatcggaaatctgttttctatcaaaaatattatcttcatttataagccgtagacacccatctaactctgcataaattttatttttttttgaaccccattgtttataatcaatgtattttattaaagttttctttaccccatgagcatatattaataatataatgctaactactaagctaagaacacttgcccaattagctatatagtcaaacatctccattccattagttaacgcattcaatatatatgacagttaggaaatagacacttgattaccgatacaaatctacaatttattcataattcctgtcaatgttaacttcaggaagaagcaaaccggaaacttttcttcctgaaggtgacaccctgaataattcgcaaaatgtgagtagtactcttggtattaaacagttgttaaggtggaaacaatgcgaagtatgtgtagaacaaaacaaggataatgttttataagaaaagaagtgcgatatcatccagatgttaatctttttcgccttttacttgctgccatgaattttgattctaaaaaatgacttttctaacaaataattccttgtatttttaattggaaagttgtaaaaaaatcttatactttaacttgacttattatggaaaaagaacttgttttaggtattgattacggtggcaaatatactggtcttgcggttgttaaccagaaaaataatcaagtactttatgcacgaacagtaaagatgcgtgatgatgtcacagacattcttgcaggtcgcagagaacaaagaagtttaagaagaacactacaaactaagaaaaaacgtttaagagagctcaagaactatcttgaaagtattggtggtatatatgaggaatcaagtgggacatttacaattgaaccttttagaacagtttacagtcttgcccataagaggggttatgattatgccgacctccctgaagaaaaaacttctgaagagatagaggcaatggatgctaaagaacgaaaacagtgggaaaaagagaaaaaagaattggaggaaacgcaaaggaattcaagacatagagacgaagttcttagagatgtacgcaacgtaatgactgaaggtaatctatcagaagagcagataataaaggttgaatctattttcaataagcagtaccgccataaaaggtttaataaccgaatcctgactaagtgcaaagtctgcggtaaaaataccccattaagaataaatgttcgggaattgctacttgaaaatattgtgcgttatttgcctctccagaataaggaaagagaattgcttaaattgactatcttgaaagggcatcagcaggatataaatgaaatctttaagcacttccgaaaagtttacaagataactctaaaccaaaaagattggccagggaagaatcttattgatatagcacggaatcaattgcggggcagattacttttctgtaaagtacattttcccgaaaatgaaaaatatgtttctattgaaaaaaagaccttcaggcttgcaccatccttaaaaacaaagatagaaaatgttctgtcggttatcaaagatgacattttgcctaatttcactttaaacaacgttgttatggaaagtaataactttgatatagcagctaagacaaagggaaagaaacgattacttaaagaagaatacagtaaaggacacagagagagtggagaaaccagaaaagaagctcttcttagagaaacagatagccgatgtatttactgtgggaaaggaatagatttatctaatgctcatgaagaccatatttttccgagaaaggctggaggtattaatatctttggcaatttagtcgcatgttgcagcgtatgcaatgaggaaaaaagaggaaggacaccattagaaagtggcattttgccaaaaccagaaattgtatctttcataaccaatgacttgaagaaaaagattcttgaagatgcacaatatattaatactttagatttcaacaaatacatgtctcatgcctctattggatggcgacatatgagagataggcttagagaattaactggcaataaagaattacttataaagcgacaaagtggtatctatactgcatactttagaaagtggtggggatttatcaaagaaaggggcaaccacggacatcatgcacttgatgctgtaattcttgcttcaaagaagagctatgcagaagatggcaaagtagatatgactattaaaccatgtggcgaagatggcaaagaatttgacatagaaagacacctttctgagatgaaagagttcagaagagataaagggggtaaaagtgctcctcttcacgataggaatcctttatcttttaagaacgatattataactcgcagatttatggtaactgaaattgagtgcgggaaagaagccgttattatttcggaggaatatcgtaaaaaactaactgaggcttttaaacgctttggtattgctaaagggaaatatttgacagatgaacaggcaaaggatgctggattctatttaaggaaaaatggcgaaggggtgatgagtcttaaatgcgaggtaaaaggaactggctataatcagatgattagaataaaaaataatatttttaaaacaaatgttcataatgttggcgtggcagtttttcttgatgaaaaaggcaaaaaaagagcttgtgaactcaagaacccaagactgtcaaagcattttgtaaagccagcagagcaggttaaaggaaaagttatattcatcttgaaacgtggtaatatggttacagttgaaggtgaagaaatgatatatagggttaagaaacttggtacttctcctgttatagaagccattgtcggtagtgatggaaaaacaaggactgtatcagctactaaactcttgaaaataaaccatactaaaaaggtttaattaaattgtcctaacccacaatgaaggtgccatttctggcgtagaggtttaggctggacaaacccaaacgtacataccctcaccgtataggcgttttacaattaaggctcttttaattttcgcaaaccattaaggatttatttcaaaaaacaacttttctaaacttttatgaattatcactgacaaaacctttgaattaaaatgaaaataattccgttacagtcgttgttttcaaagagccttaactgtaaccccatatatcagttataaacactatattctgccatgttacagtcgttattttcaaagagccttaactgtaacctaccagtgagccgattcataaggcgaacagtaaggcgttacagtcgttgttttcaaagagccttaactgtaacctcctgatgatgtacctccttttcgacagggcgggatttacagtcgttgttttcaaagagccttaactgaatttgcaaacataattaaagtgtaatatgattacaccatattgcctaaaaatcaatagtattataatagaataaactattatataataagtcaatataaatcctaaattgtatagtattttcaaactgttatggggttcaaagaattatgagtttattaaaatatggctatttgggcatgttttttcggggaaagggggtctcacgtacacgaaaagctaatttttcagaaaagcgtaaaagatagctttccccataactgagacaaaagagagattgatatacttttggatttgtgcatataatggcaaaagtgggggattcataactatgatacatgttgctccatctgattgcagatagttaaatgataacttctaatacatgctatccatttggtgggaaagtcgcaacagtcgaaacaccatcatttaaatgcatgattagtacagccaaagaaggcgttttcaagaaacagtccgtagtttttcttgatattaatatgggcatggctaatcgccgacctgtcgctaagctgcattacaatgacatactcactaaagaacaacgtttgcaatttcacgatatcatagtaagcgtgatagatgaatccggcatgaatggactatctctctatgatacactcagggacttgtttcaaggacttcgcaaagagggcattggttctgggttttttacagatacccattgatgacggctgagaagtgaaaagggaataacttaaaacaattgtggagacatacttttcaaaaaatccccttccctttacctttgaaatagtctgttgagataaagtgaaagaagaaaaaataagggaagacgttagggattatttaaactcttttctcattgaaggtggttattgttttaatgaccctaaaaaattgtgtttagccattaccagtttgtcgaacgacatatatatcttcagcaaaaaagatttccttgtgcatttgttaagattcagaactgacattgaaaatgctaaaaccttcaaagaagcctcagattcttttttaactttattaaaaccaatatctaaggagaaacctgctgattttattcaagagacaaaaaagcaaaggatgacaagggaagatgcctacggaggatatgaaagaaagagtgataaatacaagggcaaaaaaagaggcgatatttggaatgttattaaaaaggcagaagatatactgactggtaagaagataagtgagtgggagcgcatcggaataggtgaactcgaatggttcaagcttttttcaaaactctcaagaggaattgctaaaggaattgaacttcacgacttagaagcttacacgggatacagtacaaaggcgatccataaaatgatagatgaacagtgtgtattaaaggatgtgttggatatctttaataaaaagattaaaacagcattagcaaagcaaaaacctgatgacaggactttcttaaacacgatcaaaggagtatggggtaaaaaggcagcattggtcttagagaattgtacttatgaaggggaacgaggccgtaaagtctctaaggagactataaaggagaggcataaaaggataagagaagttatttacagtgggaaaattgaaaaggttaattcagggacttactacagtagagactttagagtatttctatgcgtaatggctcgtgttgtttatcacctcttgtttgaaattccctttttaggcgacaaattgaaaagagaagcaatatcagagtttagaaattttgcaaaaaaatatgatataaaactctctaaattatctgacttccaatttatagaaagcaaacccacatcaaaaggcttacccgacataaagcctcataaatagccattcttcctaatagcacgaaatagattcaattccgttcctttcagatcaatcagaaatccgacataataccaatatatgtaaaaaaaagatttttgttccaattttgttcccgaatccgaaaaaaggaggtaaaagaataatgggaaaacacacctttgaagatacttcaatagtagcattcttatctctcaaaaattacaaggtaacaccacagagaacctatgacggaaaagttgtctttattgtagaaggcaaggatattaacagggcattgcaggagctttatggcaattctcaagtaggtgttttggattttattaagactttaaaagccttacgctcaagtatctttgcactcaaagcagggggtgaaagatgaaatctatacttgatcagaatttaatcactgtatattcattcccttttgatatatttccatcaaagctattgaatgtcattaatgtactaagtgaagccttacatgtggagcctgaaattgtagcaagtgcaatgcttaccataataagtgggacaataggcaacactgtaagaatatcccctaaacatggttttgaagtggcaccctttatatggctgattatcattgccttaagtggttatggtaaatcccctgtgattcaaacccttctaaagcacatcaatgaattgcaggcaaaggcttataacgaataccaaaagcagtttcaagaatatgaaagaagacttagaaaggctaaacaagatgaaagcatagatataccagagaagccaaaattaaaacatcatgttgtatcagattgtacggtggaggcattggctaatgtgtttgagaatgatagcagaggggttattagctatcaggatgaaatagcaagtttaattttaggtcttgaccagtacaaagtcaaagggaacgatagacagcattatttggaactatttaactgtgatagctggaagatagacagaaaatcaggtgtcaagtttatccacaacacaggtacttctattattggtgggatacagcctaaagttatgcctgacgtcttcaaagtaaattcttttgatgatgggtttctaccccgtttcttattgctcaatgctgaaaacagacccatgaagtttagcagacaagcaattattgaagagattatatcttactggagagacctattaaattggtgctatgccattccattagaacatgatgatgatggattcataaaaccaaaagttttaaccctgagtagcaaagccttagacatatgggaacaattctataacgattacggcgataagatgcctttcctttcagaaagggcaagggtctttatcccgaaacttatagcctatcacagtcttaagtttgctggagttttacatgtcatagaggcattcagcaaaggtataacatccataaacaacttaatcgaggatgagacaatacatcacgctattgaattaataaaattctatgctggacagactattaaggcattgaaactgtatgagaaaccagaagacactctaaatgaatttcagaagagactcattgaaacactacgtagcttgaagggagaggttaaaaacgggaaattacagttatcaagaattgttgagctattcaatcatggcgtacctgaaggtgtgagtcatactccagaaggaattagcaatatgttggctgatttggggttaactactgaaaaaagcacgggcaattattcatatcttttatgggaatctgagaagatacaaaaacttttttctaaaacaaccgtaactactgtaactactgtaactactgtaactgataaatcaaattcagaaaacaacaaagttactgaagttacggatgttaccgttaattctgatgaagaagttatagaccttgaacatgaggagtttgaaatagtcaaatgagatacaaagtcttagaaagtttcaagattaaaacgttaaaaaaggaaatggaactgcaagcagggcaggttgtcactcttcctcatgacaaggctatcattctaatcagtgaaggtaagattactccctttgatgatacagcttatagggtctactcggagaatctacatgcttacctttgggttgtagaagatgataaggatattgaccgtctcagagaacaaggcataaaagaagctatctacacaagacaggaaatagagaaactaaaagatattgataaagactctctcaaggtaatccacagagttaaagaggtctttgagtcatcaaaggtcgtggagataaagggctgtgaagactgagcttgaaacacaggagattgaggcgatagccgataaggttatcgagaaaatgaaaccgttgcttgctggtaatggtaagtcagaggatgatgttatctttgatgtagaggggttaactcaatatttgaaggtatctaagcaatggatttacgaaagaacacacctgaaaaagatagcacacttaaaaatagatgggcaattgagatttaggaaaaaggatattgataaatggcttagctcttataatatccccgctataaacacaccttgaaggattttaaaggctataaagtaagaggaggccataagattgtatgtccgataacctatataagaaacccttaataataaacaatctacaaaccactgtgctttaaattgtgcactaaaaataattcttgactttctaacaatgttagtatatgatttagatatgcaaacctggacacctgaagaaatagagcaattcagaaaagacaacaagctaagcagaagggctttaggtgagcttttaggtgttacaggcaattgcatatatcaatgggaaagggggttgagagagccgagtaaaacaacaaaaatacttttatcaaggattgagcaagaactaaaaggaaagaaaaaacaaaaaggaaaggtaggtgaaaagaagcatggcaaaaagtaagcatcgggggatattcctgagaaaccgcatttactggattagatatgcagggcttgatggtaagatggtttatgaatcctcaggcaacgaaaagtttaaagatgctgaagccctgcttatccagcagaagcaatctatcaaagaaggcaagcaacctgagattaggaaaatctccaattacacattcaaagaactttctgagaaataccaatcatggattaacggcagacagaaatcagccagaacaaaaggctatctcatcaaacagttggttgaaaggttcggtaactttccgattaggagattcaatacggtgattgtggaacaattacagacagaccttatgaatagaggattaaagaatagtagctgtaataaggtcttgaatgttctcaaacatatgtttacaaaagctgtggaatgggagatggtagagtctgaaacattaaagcgtaccagaaaagtaaagctattaagagacgatgggaaaaggttaaggtatctctctacagaggagtgtcaagccttaatcaatgcctgtgataatcacctgaagcctattgtagtaacagcagtgaatacaggcatgagaaaaggtgaaatactctctcttaaatgggataatgtagaccttagacatggcttcattctgttagatgtaacaaagaatggagagagaagggaaatacctattaacgatactctcaggataaccttacagggtattacaaggcggttagatattccttatgtattctatgaccaaaatatagggaaagcctatcaggatgttaagagaagctttaaaacagcattgagaagaacagggataagagattttcatttccatgacctgagacatacctttgcatctcatcttgttatggcaggggtagatttaaccacagtgtcaaggctgttagggcataaaactttaactatgaccttgaggtatgctcatcttgcacctgctcacatggttaaagctatggatatcttggacaataccttaaatggtaagtttagctcagtaggtgaacgtttgcatgatgcaaactatacaaaaactatacaatcggcagaggcaaggtaagatgaaatctcctaagtgcttgaaaaatatggtaggcacgaggggtatcgaacccctgacctcttccgtgtcaaggaagcgctctaccactgagctacgcgcctgtaagactttataaaataacattttacccattactcctgtcaaccttgagtttatatcttttagtctcttttgacagttaatcatctctttttatatactttc

SEQ ID NO: 43 - DNA, 22208 bp, Nitrospirae bacterium GWA2_46_11
  misc_feature (145)..(264): n is a, c, g, or t
gcttattcaggaacttaacggcaacacgacaggcgataggcaagaggctataggcaatgggcgaaagacaaaaggcatcggcgcattgcaggcaaaacttaaagaggcgagggaatccgagtataaggcaaaagaagacgccttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntttagacgcccttaaaaaggaaagggagaagggaatacttacggacatagtggacagggaaaagagcatcagcgccatagaaggagaggcgataaaggcaaagaagaaatacgaactcgaaaggctttgctctcctgttgacggcactgttcacggacttgcctcatacactgtgggaggagtggtaactcctgcccaacccttggtcacggtagtgcctgaagggacgcctcttataatagaagcgacggcattaaataaagacataggatttttaaaagtcgggcaggaggcggaggtaaagttagatacatttccgtttcagaagtacggcaccgtaaaaggtaaggtaacggccataagcccggatgccttcgaggatgaaaagttaggaccggtgtataagataaaggtaactatcgaaaaattgaagatctatgcggacgacaggtatatacccgtagcgcccggaatgagcgtgtcggtagaagtgaagacaaataaaaggcggataatagagtttttcctttcgccgatcgtcaaatacgcgaaggaaagcctgactttgaggtaaaaacgacagacttagggcgataggcgataggtaatgacggatttaaattggcagcttaggactaatgtctaaaagtgagtctgaccttaagcggatgataactaaggagtataaaatcgaagatataataaaaccgaaagggaggccgaagaaagatgaagacaagaataaatagaaccgtcccgttttttgtatcgaggagtattctctcccccagggagaaagagattttggagcgcatgagagacggcaaaaccgacctcgaaatatccgccatttataccatcagcgaacacactgtaaagtttcatataaagcatatcctgcgtaaactgcaagcttcaaataggacacacgccgtaggcatcgccatgcagaaacgcttgctcgattaattgacatcgaatctaccccctactcaaatgagtagcttacattggggaaggggaatgtggtatataaaggcctgcatgtcttgcagccgaacttatagggaaggtatgaacgaattcagtacggcgtaaaagagtaaaagattgtggaaattactgctcaacagaaatgcaaaaattcctggaagactattccgacagattgcctcgactgtcaatgtccgctccgagccgattgcagaggaggatgcacagctgcatcagatgaggtatatggttcacaatatcggattgatccaatctgtgagttatcttgttaaaaaaattaggttggtttttcaacgataaggaaagagcatgccttattgagatcgaataacgtgaggcaagatataatcttgcagattactctaagtgggaggattgtatgcctgacatgaaagattttttaaatccgaaatcgatgttaacgcccggcattgcaggcggattaacagccagtgtttctttaacattggcatctgcatttgcaataagcttcaaatggtcggcacttggcatcagttttttgctggggttgcttataatgatttccatgcggaactctattcccttattgcagcgatttatatattgcatcctaaattcgctgattgttttttctatggcgttcggcgccggtaagggcattgattcgcagcctcagctcgatcctaagattattgacaaaattgtgaatacgcaattatcagtagttgagcctcaacccccttcggttttagcgttatccaatgcatatgcgcaacaatatcccattaaatcagataagcctaaaaaaagcactgctccttccggaaccgacaaaaaagggacaggcagcaatgaatcaaaggaaaataatctgaccaagaaagagatggaacagttaaaaacgtatattcaacaacaacagaattatgataagcgatggtcgtggtaagagaaaagtgaaattttaagaacagaaaatggggaggagaaaatgtgtaaagataagtcagtgagtcatttaaacgagttaggatggaatgtggtaagattacctaaagaaaatattaatccgcttttaactctttccaaatccaatagctatcttgaaactttaggagaaatctctgatttcgtgatagaagatcaacccaagcccccagaaattgtccaagaccaatcagtagcagaaatttcaggtttggagactgacaaatttgaattgggaataggactaaagtttttagaaaaattcctatctttagtgggagtagcgggattaggattagaagcaagctttaagaatgcagaatccattcaatttgcctatcagaatgtactgacagattttgtctatccagtgaagattggaaagtacttattgagtgtcagtcctgatgtcagtagcccctttatggagcatattaatgaagagggagaggcttatattattacagacacgcttagatctaacacattcggtattgttgcctatgatgaaaaaggtgtaaaaattgatttggatatttctgcattaaagcaacttcttagcgctactccacatattgaagtatctaaagatgagaagaaagttgtttcttttaagggggataaatttctgcgttttgcctttaaggcaatcggcgtatgggtggagatacgggacggcaaagcgagatttaaactgaataagccggaaggacccattgcaccaatgaaggctttaccgtccactttaattagtcccaatgaaccaacacctgtaattttcggtatgaacaccctaatcagactaaaatgaatcataataactaaaaaggagataaaacatgtcgaatcaagaatcgatcaacagaggtcttgacagggcatataatgagttgaaacctgaagatacgataaaatgcgatatagagtctgtgcgtatcgttatcttttcagatcatcacaggggagttcgcgatggagctgacgacttccagcggtgtgaaggggcatattgtgccgcactcggatattatttagagagaggttataacctcatagttctcggcgatgcagaagaattgtgggagtgtaggcccgcccctgtaattagagcttatgaaaacacattgaagcttgaggcagaattttataagaatagggaagaccgctactgccgtatttatggaaatcacgatgattactggagggatacgggtagtattaatataggtggggaacaaataacggcaagggataagctgatgataagcctgcatgataaaaacaataaatctattggggacattttttttgttcatggtcatcaagggacttttgaaagcgaccgctttgggaagatcagcaggttttttgtaagaaacctctggagaccctttcagaggattactcatataaagttgactacacctgccaaggatgcagaattgcgcggcgaacacgatacggcaatgtataattgggctttaaataagaataatgggcaaaaaaagcttttgctgattgcaggccatacgcatttgccggtattcgcatcaaaagatcatataggaaagattagcgatgagcttaaatctcttcagcagcagcttgataatgcgaggacatccggcgatccccatgttgacggcattgcagaacagacagcggaaaaacgggctgagcttgaatatcgaaaggcgcaaaagaatatgaaagaggatcagaaaggcatttttatggagaagccatgctattttaattccggctgttgttcattcagcgatggagatataacaggattggagattgctgacggaaaaataaagcttgttaagtggcccaaagataatccgagagtagaagttcttgaggatgccgacttgagaagcgatatttttggaagcttatagcattgctgtaccttaagttttttaagatgacgtttgtgaaaggagacggcaatgatttacgaatacgagatagacggcttgacggtgcggacaggcgatatcatctgcacttctgacggcgctgcggataccgatatcaaaggccagttctggcggctgctggggaaaataatacccggcgaggtcgatcacattattatttatgtaggaccaaacggtcgttgcgtagaagcgggagcaaaaggcagggtgataacctttgagattatggatactacatgggattttcagaagatgattgcaaaaaggggaataatagataccctgtatggagcagcatatccgcttcaggggaagagtctgagcgaaacggagataacaggaatacgggaagcggttgccgcgtattgcctgaggcaggctgaattggagaagccgtataatatgaatttccttgattcaaccaccgaagatgcatactactgcagccagctcgcttaccttgcatatttcaaacacggcatcgacctgaataccggcaagggaatacccgaaataccagggactgagcgcataatcttcccgcaggagatatggagcgggtgcgggcataaaaaagcgcggtaaacaagtttcgctcttgccaaaatccatgcttattttgtagaataagagtctaaatttttattttcaggagggtttgcgttgaaagagggaatacatccgaaatataaagaggtaaaggtcgcctgcgcctgcggagagacttttactacaaagtccacaatggacaatataaagctcgatatctgctccaagtgccacccgttcttcaccggcaagcagaagatattggatgccgagggaagggtagaaaagttcaaaaagaaatacgctaagaagtagcgaataaacatatcggggttagagcgcggggtcgtttgcgctctaaccttttttttattaaggagatatatgcataagataggcggacaggccgtaatagaaggcgttatgatgaaatctcccgaaggctggagcgtggccgtaaggggcccgaagggcgacatcagctttaaaaccgtagtaacgaaaaaaccgaacaggtttttcaggctgccctttgtaagaggcgtggctgcccttattcaggcgctttccatcggcgttaaggctatcgaattttcgggaaatatcgcgtatcaggaagacgagaaacctataggcacgctcggcatgggactgaccatcggtttcgcgattattttagccatagcccttttcaagttccttccgctttttctcacgactttaatcggaggcgtttctgaaggcgtttcaaaaagctcctttatgttcaactttatcgacgggacgattagggtgggaatatttcttttctacatattcgccatcgggctctggaaagagatgaggaggatatatcagtatcacggtgcggagcataaggttatttacgcttatgaagccggggaggaactcacggtcgaaaacgcgaagaaatataagccgtatcatccgaggtgcggaaccagctttttgctgatagtgatggttatcagcatgctcgtattcatgtcgatcccgcagggctggtctttttccggaaagctgatgtcaaggattgtcctgttgcctgtgatagcgggcatttcttacgagacgctgaggctttcggcgagaatgaaggataatccggtcgtagggctgatcgtccttcccggacttttgctccagaggatgaccgtgcgggagccggacgacacgcaaatagaggtggcgctggcggcaatgaacgaagtcctgaaattaggcgcggataaagaggtttgcaaacagtgttaaaaaagctgcttacgatagaagaaaaatacgaagagataaccaattcgctcgtaaatccggatgtgttatcgaatccccaattgtatcagaaatactcgaaggagcagtcggatctttttcccattgtcgagaagataaaggagtataagaagcttcttgccgacctcgaaggtgcggaggaactgcttaaggccggagacggcgatttaagggatcttgctcaggaagagatggaggagcttaaaaagaggaagcccgttgtggaagacgaattgaaaataatgctcctccccaaagatccgagggatggaaagaacgtaatcctcgaaatacgggcgggaacagggggcgaagaggccgccttgttcggcgcggcccttttcaggatgtattcgaaatactccgaatctaaaagatggaaaatagatattatagattcaagcccgacgggactcggaggcttaaaggaagttacggctaatattacgggcaaaggcgcttacagcaggcttaaatacgaaagcggcgttcacagggtgcagcgggttcccacgaccgaggcgtccggaaggatacatacctcggcggcaactgtagccgtgctgcccgaggccgaagaagtggacattaaaatagaggagaaggatttgaggatagataccttctgctcttcgggcgcaggcgggcagagcgttaatacgacatattccgccgtaaggataactcatatccctacagggcttgtcgttcagtgtcaggatgagcgctcccagttgaaaaaccgcgagaaggccatgaaggtgttacgttcgaggcttctggaactcgaaatagaaaagaaggaaaaagagcgtgccgccgacaggaagacgcaggtcggaagcggggacaggagcgagaggataaggacttataattatccgcagaaccgtatatccgaccacaggatagggcttacgcttcataagctcgaacaggctcttgaaggaaatctcgatgagcttatcgacgccttgataacccattatcaggcggagaggcttaaggaattgtaaaaatcgtcaatacgggatcgaattctgctataatatttacaaaacagaaaggggcggtacgcacatagacagcaggactaaggcattagaggcggcgcaggcagcgctcgataaaaaggcaaaagagacggtaatcctcgaactgaaagaccttacgataatcgcggattatttcgttatctgttccggcgaaagcactacgcaggtcaaggcaatcgtcgaaaacatagagaagatattaagagagcacaagcaaaggccattagggatcgaggggctgaggtccgccggatgggtcttgatggattacggggatgtgatagtccacgtgttcgaagaagatacgagggcgtattacgagcttgagaaattctggctggacgccccgaaaatagcggtagaagacaagtccgctaagaaggccgcgggaagcaaaaagcagtatgtatgaaggcgcggatattatgggtcggaagaacgaaggaaaaatatttgacggaagggattaaccgttacctgaaaatgttaaagcatatggcgaatgtttccgtcatagaggtgaaggaagagaagggcaagacgaaggagaacgccctttttgcggagggagagcgtatattgcgccaaacagcgtcttatatcctgctcgatgagaccgggaaggaattcagttctaaggatttcgcgaagcttctcggcgaaagggatcaggtcgattttgtgatgggaggcgcgtacggggtttcctctgaagttaaggcaaaggcaacgggtaaaatagcgctctcgaaaatgacctttacccatgagatggcaaggctgatatttttggagcagttttaccgcgcgatgacaatcgtcaaaggcaaggagtatcatcactgatgggcaagatagcgatacttgtatttttgttgttcctcggagtcctcggtttttttgcggtcgagaacaaagacgccgtgatcatgaaaattcccttcggcgacgcttatgagatcccgaagatagcgcttgttctcctttcctccaccataggcgcgcttgccgtccttatcgtgttttttataagagatacgaaacgcgtaatcgaaaatctccagtatcagaggaggcagaagaaagaggccaggattcaggaattttattccaaggcgttgaacgccataatgggcgataaggaggaggaggcgcgggaagccctcggcgatatattgaaggaggacccggaatatatagacgcccttttacggctcggcgacatctcgctcaataacggaaaatatgaaaccgcgttcgattattacaaaaaggtgaaggacataaacccgaagaatctacaggtgcttttttccatggaaacagtgatggaaaagctgcagagatacaacgacgcgtttaattacctcgaagagatactcgatctcgatcccgaaaatcttaccgggctctacaagaagcgctctctcctcgaaaggaaagagatgtgggacgacctcctttccacgcagaaggcgataatcaagctcgaacataacgagcacgacaggcagcgggaagagcgcaggatgctcggttataaatatgaatacgcccgcgcgagccttgaaaacggagatgttgaaaaggcggaaaaggcgttcaggacaatgctcaagatggacggtaattttgtcccggcgcacctcggtcttgcggaagtcatacttatgaaaggagagaccgaggaggcgataaactttctcgaaaaggcgtacgagcagctcaggtcgataataataatcgctcgccttgaagaccttctcatcaacgtaggagaaccgggcaggcttataagattctataaaaacgccatagctaaaaatcctcaggacaacgggctgagatttctcctcggcaagctctactacaggcttgaaatggtcgatgacgccatggagatgctgaattcaatcgataccacggtcttttcggtgcccgagctgtatagtttgaagggagagttatatataaagaggagccagatacagaaggcgctggacgaacttaaaaaggcatgcggcataaaaaggccgtttaagatccaatactgctgctcaaactgcggtttaaaatccgaagactggtcgggaaggtgtcccggatgcacggagtggaatacctacaggctcgacgtttatggctcctgcaaagcgtaaagacagcgatgagttatcagtgatgagtgatgagttaaaaaacaaaaaaaaatcaaagacgaaaagtaaaagctcaaaagcaagcacatcaccgtcgttaaaagtcgtactcgatacgagccttttcgtaaatccggacgtcaggacaagcctcggagcaacccccacagaggctttggaaacattcctttttctggcggcgcagatacatatactcgaattctacatgcctccgtcgatattcgaagaactcctgcattttgtggagagggacaagataccggccgatctccttcttatcttgcatcagaaaccgcctaaaaaacatgaactcacgactcccgcctttctcttatacgaattgattgaggacataagggaaagggtgaacaaggggctgcggatagcagaaaaggccgtaaggagtacagatcaaaaaaagacggatgaaatcgttcaggatatgagaagaaagtacagggacgcgcttagagaagggatcatcgacagcaaggaagacgtcgatctcctgctgctcgccatggaacttgacgccctgcttatcacggccgatcaggggctgataaagtgggccgaaaaattaggtgtaagatggctgttcccggagaaattcaaggaatatttaatgagcgcgataaaaagaaccgaacttctttccgcaaaagaataaagtgtagaaacaatgtagttttgggaagattctttgatggaaaaagaacttgttttaggcatcgattatgggggcaaatacacaggccttggggtggttgaccgcaggaacaatcaggtgttgtatgcgaggaccataaaaatgcgggatgatgttgcagacatccttaaaggacgcagggaacagagaggtatcagaagaactcagcagacaaggaagaaaagactgcgggagcttaaaaaatatctaaatagtataggatatgataattctaccgaactatttaaatctatttatagcctcgcccataagaggggctatgactatgccgatatgccaacaccggaagaaatagaggaaatggatgagaaggaaaaaaaagaatggaaagatacgcagagaaattcaaggtacaggaaagaggttttggcagatgtccgcaaggtaatgtccaatggtggagcttcggatgaacagataaagagggtagaaagtattttcaataagcagtatcggcctaagagatttaataacaggattttgaccaagtgcaaggtctgcggaaaaaatacgccattaagaagaaatgtgagggagttgcttcttgaaaatatagttcgcttcctgccgttagaaagcgaactaaaagaaactcttaagcgaacaattcttgaaggacagcaaggaaatatcaataaactattcaggaaattgaaatttaaccagaaagactggccgggcaaaaatcttacggacatagcaaagaacaaactgccgggcagactgcctttttgcaaggaacattttgcagagaatgagaagtttaccactatagagaaatcaactttcaggcttgcgccttctttaaagacaaagatagaaaatgtcctgactgtgattaaagacgaagtaatgcctaactttgctctcgatagggtagtaatggaatctaacaatttcgatatagctgcaaaaacgaagggcaaaaagaggttggcaaaggaggaatacagcaaggggcataaggaaaatagagaaactcttatggaatcccttttaagggagactgatggaagatgtgtttattgcggtaagactataactttagccgatgcaaataaagaccatatatatcctaagaaagcaggaggcagcaatatctttgctaaccttgtggcttgctgcaggagttgcaatgaaaataaaggggggcggactccatcggaaagcggcattatgcctaatccagaggtcgttgcaacgattaaaaacgatttaaagaaaaaaatcctcgatgatgcaagaagcataaagcagttagatttcaacaaatacatgtctcatgcctcgattggctggaggcatatgagggacaggctgaaagagctaaccggaaacgataaattgcctgtagagcggctgagcggtattgttacagcatactttagaaggtggtggggatttaagaaggaaagggctaacgataagcatcacgcgcttgatgctgttattcttgcttcaaggaaagattatacggatgaggggctggtggctatgacgcttaagcctgcaaattcggatggacgggagttcgaccctgaaaagcatatcaaagaatcggaagagtttaagagaaacaagggcagcagagggtctgccctctacgacaaaaacccgttgtccattaagaatggcaaaattgctcgaagatatatggttacggagattgaaagagggaaggaagatgctgttatttctgaggaatggcgtgaaaagctgaaagaagcatttgaccgcttcggggtcagcaatggaaaatgtttaactgatttacagactaaggaagttgggttatatggtcagaaaaatcctatgagccttaaatgcgctgtcaggggcgcaggcaaagggcagattgtgctgataggcaacaatgcatttaagacgaatgttcataatgtcggcgtggcggtttatctcgacgaaaagggcaagaagcgggcatgcgaacttaaaaaccagagactggcaaaacactttgtagagccacaggatgagattaaaggaaagatattatttaccttgagaaaaggggacacagttaaggcggaagatggaaatatttacaggatacttgaacttggcgaaaggcctgttgtagacataaagtgggtaccaacatctgatggcaagaaaaagagagtaaagacagctatacacgcaacaaagctcacaaagctctaactgaaaatttttttaatgtcctaacccacaatgaaggtgtcattatcgactgtagttttagtttggcttaggctggacaaacccaagcgtacattccttcatcatataggcgttttacaattaaggctctttttgattttcgcaaaccccctggggtttatttcaaaagatggcttttagaaaattattgaaaattttctctgaaaacttcttgaaaatactaagctataaggacggtttcaatcatcattttcaaagagccttaactgtaatgtgctgccgtcctgtgagagggctatctgcggtagtttcaatcatcattttcaaagagccttaactgtaacgctcatataagcaaggtctgtccgccggagaggacggtgttttctccatcggtcgcgctactgaaccaactgactgtcagttagacgaaacaatcagaacagagtccctgccggaaaccgtttccggttgtccagcgaacattgagaaagctgcgacgccagatgcaccttccggtgggaagtcctccgctcaaacgcctgaagcgtttcgcagcctttctgaaacaaatcaaaaaagctgagacaagattcctttaaaatagagcatctcaaagaaaagtccctgcgagcaatgcgcttcttgaaggaagcccctctctaacgtccgaaacgctcagggacactaaaatatatctcagaattgtaaaagagcctaaactataatatgattacattatatgcctttaaatttcaagagctttagcggaggtggatatgcaaagcaatacttttaaaatttcacgcatccatcctgaatacgtgcctaaagaaaaattgcctcttgcccaattcaatctttttacaaacgaagcggagagcattcccttattgatcgacaaagatgttgacgacgagatatttatcgatgaagggatttctctgataaaaagcggcgattcatcgcagatagtgttatcggggttcggcatttttttaagcaaaaagagcgagcgtcttattgtaaagaaaaacactaaagttgtttatgagtttccctttttcaggctgaacgaagtcatcatggcttcccgcggcatatcgctgtcgtcggatcttatagaggaattgtgcgagaggggtataagactgaattttctgaacaacaacggcaagccgtacgcaatgctttcttctccgatgctctccgcaaccgtcatatcaagaagagagcagattattgcctttaccgatacgcgcggtcttcaattctcaaaagctattgtggagggcaaaatcagaaatcaagaaagacttttgagatatttcggcaaatatattaaaactgttgacagcgccaggtttgacagaatagaggagataacagatgtcattaggaagatgagcaggcaggtggaaggtattcacgggcagagcattgaagatgcaagaggcagactgatggcaattgaaggcgcttcaggtaggctatactgggacggcgttaaggagataatcggaaatagaatagagtttttcgggcgggaaacgcggggagccattgatgccgtaaattccctgctgaattacggctatgggattttatattcccacgtctggggcgccgttataaacgccgggcttgagccgtttgcggggtttcttcatgtggacaggcccggaaagccgtctcttgttttggatttggtcgaagagtttcgtcaacctattatcgataggattgtaatcgcccatattaatcttggagaatcaatcgaaattagaaatggcctgcttgaagctgagaccagaaagctgatagggaacaaaattattgaaaggctggagagtcaggaaacacttgacggcaaaaaatataaaattaggtcgataatccagatgcaggccagaaatttagcggcctttctgaacggcaaaagagaatacaaaacgttcagatttaagtggtaattgatggaagaacatacagtctatattttttacgatatagaagatgacggcatacgcaataagattgcggagaagtgtaaagactatgggctcgaaagaatacagttcagcggcttttccggcattttaaatagaaacaagcgggaagaactatttctaaggctcatttctttaattggcggcaaatcaggcaaaattcttatgcttcccttatgtgaaagggatacgaaggtaagaagggagtttatacaggaaggacaagatgattcaactgagggtcggtgatctcaagcagttcgtatattgtccaagagtcgtgttctacaattatgtgatgcctgtcgagaaagttccaaccttcaagatggagcatgggaagatagcagaggataaaatagacaggcttgagcacagaagaaaacttaaaaagtacggcttgtccggcggggaaagggaattccacttgaatttatattctccgaaatatgagttatcaggcaagattgatctgcttatcaatactgtcggttcttattatccggttgactttaaatataccacctcgcagccgcataaaaatcatatctatcaacttttggggtatgccctaatacttgaagacatatataaatgcaaagtcaataggggatttgtatatataattcccaaagaggatgcaatttcttttgacttaacggatgatttgaagggcaaagcagaaattttgcttgatgagatcagagatatgatagcattgcagcaaatgcctgcccctgcaaactccagaaataaatgtgacgactgcgaatataggaatttctgcggagatattttttgacaattataaaccgccgagggctttatacagccctatcacgaattccgtcatcctctcgtaatcgagcgtttcgaatgtatctcccggcgcatggtaatgaggattcctgtaaaatgccgtgtccgtgatcatgaacgccgaatagccgaactcccagaagccccagtggtccgagaaattaacgccggggatcagcgaaatcccgttgaacgattcagcgggcagtgaagatgactcttgaaaatatctcttcacttttttcgtaaaagacatggagcggatatttccgacaaacgtaataaaattgcccttgtcaggataaaaccatttgaatatcggaaaaggataatactgagagttttttctgtcggagtaatatccgagcatttcgagggagatcatgccgcagacttttatgccttcctgtttaaggcttttagcataaacgtagcttcccatatgttttgtcataaaagtgggaggctcttcaagactgaaggcaacaaagcggaccgtcctttggaaagattttgttacggcaagccgcgccagttcgagcagtcccgcaatgccgcttgcattatcgtccgcgcccggagtgcccgttacggtgtcgtaatgcgcgcctattacgagaatcccgtcttgcgggtttccggcgccttttatttcgcaggatacgttgtaatatgtattgccgagatacccaaacggctgctttctaacctcacagccgtaagagcgaaattttttttctatgtaatcggcggccttgttgagcttatctgtatcgcgataactcctctcgcctatatcgtgggcgaggcgcttgacggtttcgataagattagcttttatattttccattttgccgattaatgttcatagctttataaattctttcggcgctaaattcgtcctcattgtccataccgatgcagtgccgtttttcaagcgatacacaaccagcgggtcatagccgtattgctccacaaagggtttcagaaattcgcgcttttgcactatctccttctttagttcaatatcttccgtaagttcaacgaccccgctgactcttatttgtataaggttctcaaagctgccgttgttaaagctcagctcgaccagcgggttttcggtcaattgtttatgcaagtctttcacctttcccgtgtgaaacaaaataccgttctcatcagccctatacatcagcataccgcgaacgcggggcttatcaccttcaatcgttgccatgtgaaataccgggtttgcattaagaaactccaaaatttcagtcttgttcatataagcttcctcctaacgataaatgttttcaagcctgcggataatatcgagtttttctttctctgttgttctcactcagggttactccatttgcggccctttgcctatcttcattttgcccctgaaatatctttgtttgacttccgcggtcttttccttccatttgtccacgccgtgctgtttaatgtagcacaaagcaaatagtttaatgttgtgcggcaggtcgccggccttgtccgttgcgggatttaatttggcgcaggggaattccgcgcactcgaagcagaagtcatgtccgcgtttgctgatgcaggcataggtctcgcatgtgccctcaacaaactggcagcgtcccttgagcggcctgcatccctgacatgggatgccgttccagttaaattttttggtcagcatttctctccacgaaggatcgtcctttactttgtacgctgggcagtcaccgcagtatcccccgcacggcgcaactaatgtgatgtcatcttttgtttccattcgataagcctcctgtagccggtctgtatacctttgcctggttgtcgcgatatagttgatcgaaaagttcagtaaattgggattgtccggccatcatatcctcttcttagattacacagcactaagctgttttttcaattcaggagtgtgagttaacatcgggacagcgaccaggaaaggccgctgtcccgttgcggtcatactaacaacagcccttgtctgattttgtcccgcagcaggactggaacatgttcttcatatttccatccgtgcagcacttctcaaaaatggttttaattttttcttttacgtcttgaccggtgatctcaagacgatacccattctcgagttccgtaacgttagcgctacagcattgtttctccattattattcacctccttccagtcttttattattcgagaacactcgaacaagaccgccaaaaaaaactattccatgaacttttttatggaatcgatcacgtccttatttactctgcaacgatagatctgcccctcgcgctcacatgttataagcccggcattttggagttccttgaaatgatgcgatattgttgaccgtgaaagattcatgcaatcacagatctttgtaaaggccttttccatgcttccatcagacgcgcaacatgttgcccctttaatgcgaaattccgccccggccttggacccctcgattccgcaacagtttttgtagagcataatgaatatcttcaatcgctgctcatttgaaagggctttaaatatttttgtgtagttagatagtttcacaacattagaataaacttatcgtgcgtttttgtcaagcattttgacgagaggtaaaagctttggcgttattatcaagagtatgaaggagtagggggataaaatcggctgaaatttttacgaatgatataccgaagccttatgaaattcttcaatcgccatatccttatcgtatgagaagccggcaggaaaaatgcccttctttttgcaatacacaaccatctcatccagcacagccatgagttcatctttcaggttgttcacttcccctaagccgtcacaacccctctgcctttcttttataaaactatatatctgaacgtagtccttagcttgtgcgtagagtcctgaaagatgagaatcggattccgccattttagtaattgcatcaatcatgcatacacctcctttcgactgtatagacgaaggaaagtgggaaaaggatacacccttattttttcttgtggaattttaaggtgggttctttgcggtcgcaggcgaattcgttccgctcgtcgcggtagaaactgcagtgcgcttccagcgtttttttcagccttgcccgcgcacggtggagcctgatcttgacggtatcaaggcttaccccgagaatctctgctatttctccgtttttgaacccttcaagctcgctcaggacaagtatggtccgggaatgagggggaagggcatctacatacccccggatacattcgctcatttccttgcggatcaattgcacatcggggggaggagtttttttatcataaaaaacatctgtttcttcattatccataccgagcggctgttcttgtctgaacgacggactgcgcatcctgtcgactgcagcgttcgttgcaattcggtatacccatgtcgacagccgggaattttttctgaaatccctgagtccacgatgaacctttacgaaaacttcctgggctgcatcctcggcctcgccggccccgatcaaccgctgcaggtaacggagaacaggcgtcctgaattcattataaatttgttcaaatggcgtatcgttggtcatcgattgttgatctgctttttaatctcctgcataggagtcattccttccccaaaattaaatcaaccatgattttacgaggttggcagaaaagtttttttactctttccctatattcttcgggtgtaacgtgttcctgatgagttcctatcacgattttaatattcgggaatgcctcttccaaagccgatttgtatgtctctttaaacgggcatagcgccttcatgcagtaagtaaagtgtatcgcatccacccggaattcagtcagtgcccttatacgttgcaataatttatcggcgccgctaagggtagggcagcccggacaattaataatccctaccaaatctaacgggtcatcttttggatagtccgcaaatgttcctcttctttttctgagatcggcaagacagcttgccgacgaacagcccaagtcctgcgtcgcattgctgcatgtcaatattccgattcgcgccatataatcaccttctcgtttacagtttctctaaaaactctttcctcacaggcgaccacgcgtcaacggccttgcaaggcatgttgcctgtaaaggccgaatgaacggcgtttgacggtatcgcgattacatcccctgccttcaaggtagttgtttctccttcataggtaaaggtcaattccccttccagtatcagggtgatctgttccgcctcgtgcgaatgctcaggaaattttgtgtccggctccatctcaaaatatgtaagcatcgccttatcgagagctattgcccacatcgaggctcccgggacattgcttctgagctttaattctcctttgcgataaatatgtatctcgcccattgcttctcccctttttagcgatttcatttttttagatatttcatctgaatattccagagtataagccccgcgccttcgggtctcgaaggcagaaattttctaatttcagaggcgataaattcggcattgtcggatttggccgcaagctctttggcctttttatcaaaggcaataagatagtctttcatgtcttctatatctttttttgttgagaccggcccgtgccccggaattattttctcgacgtccatcgtcatgatatagtcgagcgtttttaaccatccctctatatcgccttctcccatgaacgggtgataaccggtaaatagaatatcccccgcgaatagaatttttttgtccggcagatagaccataatgcttccatcggtatgcgactgcctgaaatagatgagttctattttccggtcgccgaggtctatttccattctttcgttaaacgtcaaaacagggtatgcgatcttcgtccctttcacatccttttcggtaagtccgtaggtatttgagtttttcagggctgtttcgccgcgaattttcagattattaccgtcattagtgtgcgagatgatcagcgctccgagttttgcgaattccgagttgccgaacgtgtggtcgaggtgataatgggtgttgaggacatatttaacgggtttcttcgagactgaccttatgtccctgataaaccttttcgcctcttttgcggatatgagggtatctactaccacaatgccgtctttaccgataatgatgcccgcattagcgccgtagctgttctgcggagtgctgtttttagtatccacatacgaataaacgtcgtcggctatctttgtcagtttctccgcggcaattgcaatttgaccaaatgctaaagaaacgatgaatacaaatacccaaatcctgaaaatcatcaaccttttcataacatcctcctcgttttttattttaatcctttaccgaatccttcaacagtttcctgaaaacaaccatattcgattttgtgaagccgtgcttccgataaaattcatgcgcctgctcgttatcattatcggtaagcagggtgatccttccgcatccgttattaatcgcaaattctaccgcatgcttaattaaacttgtacccacgcctttcccccttgaatcgggcgatacaaccatatcttcaagaagggcgactttcttgccgagggcggtacttatcgtgaataatacgactaccattccctgaatgacgccgttagattcgcatacaaatacagagccggtttcagggttcttaacgatcatctccaatcccctcctttgagccgtcggattgggtttaaactcatgctcttgaccgaaaagaaggccgagaagctcaatgcaagagccgagatcgtctgtatttgcaggtctaacggttttcattaatatgctttctctcgtccgatcacttcattctcaagaattcttcaatcaccggacacaattcgctccctttttcttcctgcacaaaatgtcctacatcatcatattttattgttttggaatcggagaaaaggcttatccatctgctcagttcctgctctctgaaagcaatgtctttcattccccataaaatgagagccggcttatcttttattttatcactctgcgaccagagaaaatccagccaatcactggaatcaataatccgcttcggaaatgtccagcaacccttccgttgctccggtatttcaagcgccttgaaataatgctgatggattgaagtggggagtttggaggtatcacccatcgcctttttcattatgacccgcacaaaaaaattaaagcgtttgatcaagaacctgccgattggtccgcccacaaatttgctgaacctctcataataaggatctcccttgacagaccacatccaggtattcatgatgatgatacgtttcatattttccggagagttgacggcatatgacaagccgataggtccgccccagtcctgaacaacgagcgttattcctttcaaatctaatttttctatcaatagtttcagatttttcgcatgatcttccggataatacgaccaattccacggcttatccgataaaccgaacccgataaggtcaacagctatgcagcgataatttttcgagagacatttaatcagatgcctgtatagaaaagaccatgcgggattgccgtgaaccataactatcggctctccgctgccctcatcaacataatgcattttccccatttcaagttcaagaaaatgagacttaaacggatactcttttctgtctatccacttattcatgaaacctccttatctctcatctcctttttcgctccgtgttcatagcctgaccgattcgcgccgttataaaccggtatttgccaagtcggtccatggagagagaactaccgccaaacatttcgattatatttccacaacccattcttttatccagttgctctcaagtaaatcctgaataacaaaagggtcgtttattataatcctatttgcatcgtcaacgctttcggcctcaaatgtaatcattccacccgtcctgtctgcaaaaggccctccgagatacttgctcaatttaagcttgtgccaatactcaatatgcaacggtacagcatcctttatcttttccggctcctttttcatgaaataaaaaaatgcaaattgttttcccatgccatcctccctgcag

SEQ ID NO: 44 - DNA, 9713 bp, Deltaproteobacteria bacterium GWF2_42_12
attgggtgggatagcatggatttgtaatccttttccctcagctcagcgaattttttatatcttggatcctttgatacatcttttacatagattggctttccttcttttgccgctgatcctgtaactccttctcccaatttgagccttaccttgccaatcgcctcttttacaaagcttgtggttgccgttaagacaagttcttttccattccagaggtatattgaggaaacatctacgcccaatctctttgcggtcttttctacgactgactggagcacctcatcaagcgcataatcagagcttgcaagcatgcttatctcttcaagggtcataatctccattttttccctgttcgccctttccatctgaaagacaatgccgccttcttcactaatctttacatgagaaaaagcatcgcttggataatatggattgatagccttgcttgctgacggcattgtcttatattcccaaaaaccgcatgcgcacacaagctttaccctgtaatcgtcaaccctctcgcttttcatcttctttccgcatttcctgcatcgtgtaataaattccattcagcgcctcgttaaaatcagttggaatttaacaggctgttgaaaaagccatcaacagccttgattacacagattagagaaaaagattacacagatatttacagaaatttcaaggagttttaatctgtgtaatcttgtcttttatctgtgtaatcccgaatagtcgggatgtaatcggagattgttgagtttttcaacaagctcttaatattcgggagtcggttcttcagtctataacctgaggtatttctttgttctcttccgaaatattaggcaagacgagttttacgaccttgccttcgtcaccaagtctgccaagtggattgccattcagcgtcacattgacaccgcctgcattccctatcagtatagaaaatttttcttttgctatccatttaaccttttcgccttctctgagagatacctcaaacggatcctgactatccacttcagctcgaatccatgtgcttttcgtggcttgtataatcaaggccatgggttcttttaagtctgccttagatgacacagattcttttgtatttttatcccctttttgtaaagccgaagggctaacaagtatgcctctatcgtcttcctgtttagaccgggcctcgtaagctatagcttccttcttgttgctatcagatacatcgttgccagtcgttagctgtatttgtgaataatcaggagcatctggtttatacttttttaagattacatatgctccagctgaaattattaaaaggatagtgacagctatagcaataagcgattgcgtagataatgtggttagtctctctaacttttcgtcttttgctttggcagcctcaacaggttcgaccttactttcagtaagggttttcatatacacttcataacgaagcagcgcatcatgtccgtctacaccaagatgtttgcaataagcctgtataaaaccttttacaaaaggcggtgcgggtaatttgctaaaatcgtctttttctaatgcattaagcagcccagcccttattttagttattttagccacatcttgtaatgagatacctctaatttctctttctcttttaagatattcgcctatgctctccattttgctaactcctaattatttatgttgaaggcttttgcctatttaatgtttattatttcaagaggtcaatatatcccttagccgaccttgccatctcgctgtcggggacaagtttaataacttcttggaatgcattcgccgcgcctttcttgtctttaatctttatgagaacaagaccgaggttgtaatgcgcatccgcataatttggcgcgttcttaattacgagtctgaaagtatcagctgcctctttatctttattcattctcatataggtaaggccaaggctattatatgcctgcaaatacattgaattgtttttaattgcctttttaaaactctcttctgcctctacataatctgttttttcgtaaaatgctcgtcccatattaaaataggcaagctctggagttgtataaaggatgtttgcgagcgctttttgggccgctgcgatagcagcatcccactcttttttttcaatgtagacagctgagaggtttgtgctggcatcagaaaattttgggtctattcggatggcctttttaaagtgctttatcgcatcatcatggaggcctttcgcaaaataagcaagccccagggcattatgatacgatgcctcatcaggatatttttcaacagcagttatcagttctttcagggcttcagggatatttctttcattgagatgaacaaccccaagcttataatgtatatctgcatcttcctttctatttgcctttgagcctgcacatccgtaaaagacagttgccaggaggcagtaagcggtaagcagtaagcagtgggtaataaacagtaactgcctactgtatactgcgtactgcatactttctttgactgctggtttctgacttccagtttctaaattcttaatcatctcatactccttagcctcttgtcggttttcaagatatatcttcaaaatgggttaattgcttaaattttctatatctatcgtaaatctcttttagtgtcagggtgcgcattctgttgaggctgaaatcctctacattaaatgaggccatgacactcccaaagataattgcctgtcttatattagattcatttgtgttgtcaatatttgcaagatatcctatcagtcctccagcaaaactgtcgccagcgccggttgggtcaaatacagattccaggggataggcaggtgctgaaaatatagaaccaccattgaacatcaatgcgccgtattcccctctttttattattactgttttagggccatatgcaagaattttttttgctgcctttacaaggttgggttcttttgcaaactctcgcgcctcaccttcattcaggacaaacaggtcgactttcgacaagagccccttaagcgcatcaggcttgccttctatccagaaattcattgtatcacatgcaacgaattttggctttttgacttgttcgagaacattcaattgaatctctgggtcaatatttgcaagaaacacatagggtgattttgtatagaccgctggaatatgaggtttaaattttttaaaaacatttaggtgagttgcaatggtatgggcttcgtttaaatcatactcatatcgcccctgccagcgaaatgtcttgccagctattttttgtaagccgtttgtatctacccctttttcttttaaaaaattaatatgctcatctggaaaatcttcacctataacagctacgaggctgacatctgtaaaataactggcagccgtagaaaaataagtggctgaaccgccaaggacattgtctgcctttccaaaaggtgtttcaactgaatcaagtgcaacagaacctacaaccaggatgcccatcaaaatcctcctgaatttcaagtaggcagtatgcagtaaggagtaagcagaaagaaacctgcctactgcctaccgcttactgcttactgttttttaccattattcatccacactgtcaattaatgtggaaagggcaactatctcaacttccctcttgagcattggtaccatctcttttatagctaaaatggtggtatggtgtggatggcctatagcaatggcagagcctctcttttttgcaacttctatcaattctcttatctgtccctttatgtaatctatatcctctttgttatctaaaaatacctgcctgctcaatgttttaagtccgagctcttttgctaatctataggcagttgatttattcgtggttttgctatccagaaagaatagatttttttcttttgcgatttccaatactatcttcataagtctttcattctctgtaaatttcgagcccatatgattgtttattccaataatatgcgggattgcgtcaatatcctttttaacctgtgaagcaacctgtttttcagacatggttgtaagtattgccccttcccctggatcgttgttgcttaaatcttttggctccatggggagatgcagcaacacctctcgcccgtttaaataagcctcctttgcagcgttctctgaattcggcaaaaatggcaatacagcgatagcaatgggggcatctatttctaatatctctttaagctgtgccctgtcgcggccaagatcgtctataattattgctaccttagccagtggtatatgcggcttttggggaacaggaggttgaggtttctctatctttgctacaactgtagatgttgaaataggttctggaggtttttcttttgtactgtagctgaggtatagaataaaccctaatatagcaatgataaaaaataccgttgcaattcccaaaacaaaaagggtctttgatgagaaagacccttttttgctccagttatttttagccattttttaacctatatagctaatccctcttagatgagccactcttatgattccttctttattgtctctttaaatatataccaactcttcagataatccaatgcccttttgagctgcatgtccgcttctgtctcttccttttcttttactaccttttcctctatctttatctttttttctttaggagattcaacctcttcggcttcaaggtgtctttctagttccttttctttaatatgttcttttattgtttctccaaccacaatatcgggttctatgccttttgcctgtatagatcttcctaaaggcgtataatatttagaggttgtaagtctaaccgctgaaccatcgcctaaaggaatgatagtttgaacagaacccttgccaaatgtctgggttccaagcacaaccgcccttttatggtcctggagcgcgcctgcaacaatttctgatgcgcttgcgcttccaccatttaccaggactataatcgtatattgggggtgcgttccatctgcccgtgcctcaaacttcatatcctggcctgggcttctgcccttggtatatacgattaaacctgattccaaaaacgtgtcagctactcctacagcctgctgcaaaagtccaccaggattattccttaagtcaaggatgagaccttttaacttgctgtcccttgagccaagcttatttagcgcctcttcaagttcattagtagtcttttcctggaaatgtgcgattcggacataaccaaacccttcttctaacgttttatattttacactcttaatagctatgatatcccttacaagtgtaaattcctgtggttctttaaagtcctcccgcattatccagatagtaacctttgttcctttttggcctcgcattagctttacagcatcgtttatggtcatatcctttgtcggcttatcacctatttttactattttatctccagcctttatcccagccctgaatgctggcgtatcttctatgggcgcaatgacagtaattatgccatcccttattcccatctctatgccaataccaccaaaggcgcctttggtttcaacctgcatttctttatactcatctggagccataaaagacgaatgcgggtcgagcccgagaagcatgccttttattgcgccataaatgaggtttttggggtcaacatcttctgcatagttatcctctacgatagagatcacgtcggtaaatatttttagattctcatatatctttggaggcacagcggaaaccctgtgattaatcccccatagaagaacgctcgttattagagctattcccaacagcgtcatgagcttacgcccttttaacctcttgaacataaatgtattcctcctgagtttatttaagataaagacttgatgcgatatgcgaaagggttaaaggaggtagcgctacctaattgccttctgccttaagcctgacgctctctgtcttatcatatgtcaaccaatttaccgggtctaatggtacacccttctgtcgtatctcaaaatacagatgaggtccttttattgaacctgtatctcctacaagtgcgattatatcgtctctttccacggtagaattaacctctgcgaggatttttgacaaacggccaaagagtgtatgatacccattgccgtgatctataatcaaaacctttccatagcccctgaaccatccagaatatacaacgtggcctttatatacggacttaacctcagcccccatcgcagcctcaacctcgatgccgttattaaaggtaacagtgtgaaatttgggatgctctacctttccatacattgaaatgactttgccagctacaggcattggcaaatgaccgcgcatagacgcaaaaccgttcgtctcattatccaaaggattattaaccgtctcattcttcaacctatctatgaatagttgaagctctctgcctgcgctttccatttcctcaattgcggcaatctgtatttcctttttcttttttatatcatttaaaaggacctttttcttgtctttttcttcttttatttcttcctttttcttttcaactgcatcttttaaagagcctatctcctcctgaaactctttgagccgtattttctctgcttctaagagcagttgattctcattgtaatcttttaacagggttgtatcatgatctataaccttgtttacatatttatatcttctactaaactcattggcaaaatttggggcaaaaatgctctgggttatgcccgccctattcatcttgtacattgcaacaagcctttgttttagaagttccatcaaccttattctctcctgaatcaactcttttattcggatatccacatcagagtttttttgctttatgacaacaaggctgttttctatcttttctaattcctcctcctttttagataaggccttgtcaagcctttcaagtccttcaagaatagcatattctttttttacattatactctaaacttctctttttttctacaatctcccttttgagggattccagttgtttttccttttttacaatctccttctttagcttggcaggttcagccgcataaccatgaatggccggggaaacgagtaaacacaaaacaagatagaaggggtaggttctaagggtcaataacaataataggtttaaattatggattgccacagttttaactttctctttttaagcagtcattggttttttatagaatttaatttatcctatctaaaacaattaaatttatactctttattctatctttcagcgcccccatttttgaatctttgccaagctcgaggtttgtaacaacagctttatttacagttctgaaaccatctttatcataatagccaaaaagctgatggagcgcatagtagtttccatctacgttgccgagatagagcatgatatgaccgttgagggctaataaggtaatgcctgggattgcggaacttattgcgttatcaatatctccgtttgccggcatttcatcaaaacctgctaacagcgtcccgacattagattgtttgccagaatgcctgggaaggtttatgcccatcgtcgcaaacacatccttgataaagcttgaacagtccctcaaaccgtttctacctccccagctgtatttctcgccaaggattttaaatgcctgttttatcacatttttcttggtatagggaagatagccaatattcacatcgctgcctttcttaataaatgcatctgcccattgaagctgaccattgcccattttttttggaaatttaataatccaatgtcttttatcttcaccgctaagcgcaaaggaacgacccatgggaatggactcaagaacctttgcacccttattgtctgcaaaaactgccactcggtttcctgttattaaaacgaatttttcagcatctattattaaatcgtctttgctatttacaaaagcaatgatattgagttttatccagccacgtacaatgtttgtctgaaagaaaccccactgtccatccctactcttgtgcaggagcgccaccacctgaggaggatatatggcggaatactgaaatgcatcaaaatctttggcagaagggcctttaaacacaggttcatcagtagggaatgaccttatatccgtccttttagttacaatcccaaatgtaagttcattcgattcatttatagcctcaaaattcatattgtctaatagttcttcataaaaggcgtcttttacaggttttcccttcttatcaaacatatttttatctactgggagagggtcttcaagcagccatttttcaagctcttcttttgataccgatgttcccatttttattacatctgccatttcattgttttcgccaagaatcaaagcgtttaattctgcaattttctctgaagaaagtattacctcatcaggattatcaagccttccaatccagaagtctgctttaaatattggaaggtaggcgcttgcttcgagcaatacgggagagagaaaaattatactgatgactatcttcaaaactttcatgccaactttgataacataatacgtcaatattctcaagtcataaattatgttcggtaaaatcaatgtctatttttgggtatgtttcaagctctggacaataaaatactactgtgatatacggagtctgtggaaaagattagaaatcctattccaacagttgatataataattgaattaaaaatcccccctcacccccctttttcaaaggggggaaggggggattgggggattgtccttatagagaggaaaaatcccccgtttggctgggcaattccaggcggttttgtggattatggagaaaccttggaagatgcggctatacgagaagctcatgaagagacagccttgaaggtgagactaaaatgccagttacattcttattcaaaccctacgagggatccccgatttcatacaatttccacagtatttgttgcagaggcagatggaacaccgcaagccagagatgacgcaaaaggaatcggcgttttcacagaagacaatctgcctaagcctctggcctttgaccataagaagatattggaagattatttcagatggaagaaggaagggtggagggtgtttgaggaaaaggcattgcagtcaaatgatggctgtgataacttgcttagctgaaaggtaattggattatgataaaagatacccaactattaaaaaaatttgaagacacaatcatgaaaaaagagggaaggctttcttttagttactctatgcgcatatttgaatctctctggaatgaagggataaaactcggaatattgcctcccaagaaaccattagaaggaatagaagttgatataaagatagcacaggtgctcaattcatgtttgaagaaatcctcgcaaggataggcgcagcgcttgttaagtgcaatattccctacatgattataggcggacaggctgtattgctgtatggagaaccgcggctcacaagagatattgatataaccctcggcgttgatacaaatcaccttaatcaacttcttgcaatccttcaaaaaataccactcaagcccataccagaagacatagaatcattcgtcaagcagactatggtcttgccggcgctggatgaaactacaggcatacgggttgactttatattctcatttacgccttatgaaacagaggcgataaagagggcaaagagtataaatattctcaatcaggaagtatgctttg

SEQ ID NO: 45 - DNA, 2044 bp, Deltaproteobacteria bacterium GWF2_42_12
ggacgatgtcgctggtattctcaaagatcgcagaaagcaaagaggtatcagaagaaccgctcaaaccaagaaaaagcggctgcgtgaattaaaaaactatctaaaaagtattggatataacgaatccacagcaacatttgaaacggtttacagccttgcccataaaagaggatatgattatgctgatatgcctgaagaaaagacttccgaagaaatagaggcaatggatgtggaagagcgcaaacagtgggaaaaagagaaacaggaatgggaagagacaaaaagaaattcaaggcacaggaaagaagtcgtaaaagatgtccataaagcaatgattgaaggaagagcaaccgaggagcagataaaaagggttgaaaggatttttaataaacagtaccgacctaaaagattcaataataggattttaactaaatgtaaggtagaagattgcggagttaatacgccgctcagaaagaatgtgagagatttgcttatagaaaatatagtcagatttttccctatagaacaatctgaaaaggataatctcaaagatgctgtgcttgataaaaacaggagggaagaagttaaatcatttttcagaaaacataagactgatgaacatattaggaagcaggtttatgatatagcggataacaagctttcgggtagaacggtcttttgcaaagaacatatccttgaacatactgagcatagcaaagaggaacgaaaggttttccgtcttgcgccatccttaaaaacaaaaatagagaatgtccttgctgttattaaagacgagatattgcccaaatttactgtcaataaggtggtaatggagagcaacaattttgatatagcagccaaaacacaggggaaaaaacgattggcaaaagaagaatatggtaaagggcccagggaaggcaaagagaccagaaaggaagcccttcttcgagaaacggatggtagatgtatctactgtggaaaatctattgacatatccaatgcgcatgatgaccatatatttccccgaaaggccgggggcttaaatatctttgcaaatttagtagcatgttgcgctgtatgtaatgagaataaaaaagggagaactccgttagaaagtggaatctcgcctaaacccgaaataatagccttcatgaagaatgatttaaagaaaaagattcttgaggacgcacgaaatatcaatacagtagattttaataaatacatgtcccatgcctctatcggctggagatatatgagggacagactaagagagtctgccggcaataaaaagttgcccatagagaggcagagcggcatatacactgcatatttcaggaggtggtggggatttaaaaaggaaagaggcaacacccttcatcatgcccttgatgctgtaatccttgcttcaaggaaagggtatagtgatgatggattggtggatatgacccttaagccaaaatataataaaggtggagggtttgactctgaaaaacaccttcctgaaccgatagaatttaaaagggataaaggcagcaagggctctgcattgcatgatagaaacccactgtcttataagaagggcattattaccagaagatttatggttacagagattgagtgcggtaaagaagacgatgttatttccgagacatatcgtgaaaaactaaaagaggcttttaaacgatttgatacgaaaaaaggaaaatgtttaacagacaaggaggcaaaggaagcaggtttttgcattaagaagaatgaattggttatgagccttaaatgcagcattaaaggaaccgggccagggcagatgataagaatcaataataatgtttttaagacgaatgtccataatgttggcgtggatgtttatcttgatgaaaaaggtaagaaaaaagcatatgagcgaaagaatccgagactgtcaaaacacttcatagaaccaccaccgcagcctaatgggagagtttcatttaccttaaaacgaagagatatggttacagttgaaggtgaagacgcaatctatagaataaaaaaactcggaaca

SEQ ID NO: 46 - DNA, 1090 bp, Deltaproteobacteria bacterium GWF2_42_12
atcaaaaatcctccgcatccatccggaatatcaaccggaggaaaaactcccgccagcacagttcaatctctttacaggagaagcagagccagtcccgcaactgattgacaaaaatattgatgatgaaatatttgttgacgatgggatttcgcttgtgcagagcggagattcgtcgcaaatagttctgtcaggttttggcatcttcttaagtaaaaagagtgaacgccttattgtaaaaaagagcagtaaggttatttatgaatttcccttttttagactgaatgaggttattgtcgcctctcgtgggatgtcattatcctctgaccttgttgaagagttatgccaaagggggataagactgaattttttgacaaatggcggcaaaccgtatgcaatgctttcctcgcccatgctctccgcaacggttgtatccagaagagagcagattatggcgtttaatgatgaaagggggcttgaattctcaaaggcggttgtagaaggaaagattgtaaatcaggaaaggcttataagatattttggcaagtatattaagaccgtagatattgacagatttaataaacttgaaggaattgctgatgatattaaaaagatgagcggacaggtagataatattgatgggcaaaatattgaagatgcaagaggtggtttgatggcaatagagggtatatcaggcaggttatattggggcgcggtcaaagagattattgcaggaaaggttgagtttttcggccgtgagacaagaggggcagtggataaggtaaactctctcttaaattatggttatggcattctctattcgcatgtctggggagcaattataaatgcaggtcttgagccatttgcgggatttctgcatgtggacaggcccggcaagccgtctcttgttctggatctgattgaagaattccgccagcctgttgttgataaggttgtaattgcccatatcaacctcggagaatccattaacatgaaaaatggattgattgaagaggaaacaagaaagacaatgggaagcaaaattatagaaaggctggaaagccaggaaacatttgaaggcaagaaatataaa

SEQ ID NO: 47 - DNA, 6770 bp, Deltaproteobacteria bacterium GWF2_42_12
gagacgattgaatcttatattaagactaaaaagattcctcctttcaatgtgagcagcccggcgcttctggaacatcgtggggcatttgtttctataaagacaaagggcaatttgagggggtgtataggcatctttgcgtcagaaaaacccctttatctcactgttgtagacatggctgttgccactgctacgcaggaccccagatttattccgcttactgcaactgaactatcactgattaccatagagatatcttgtcttacggcgctaaaaaaggtaaaagatactactgaaatagaggttgggagggatggtctgtatattgtaaaggggtattgcagaggcgttcttctcccgcaggtcgcagtagagtgttgctgggacaaagaaacatttttagaacatacttgccttaaggcaggtcttccatctgacggctggaaggatggggctgatatatatacatttgaggcagaggtgtttggcgaaggcgattgataaggccgtaaaatagcgattaaaaacttcgctattttacaaacctgcgatttgcctgctccccgatagagacactcggggacaggcatacttcgttatcgacacgctcagcgtgccttgaccaagcttagcttggtcggcgcatttttgctcctcaccgtatcctccccctgtccccctgtggggacagatttcaaatctgtccccagaaaagggggagggctcgtcgcaaaaacatgtccctgagtgcttttatcagggatggcctctgcctcgtctgacaacccccgatagaatcaatcgggggcggtttccccaaaaatcaaacgctatttttgggtaaggtatttttagtgaaaaatattcttactttatgctaaaataaaaccccaaatttgtatacgaatgttaaaaaataattctacaacaatccatgagactgattaaatctatacttattattttaattatctgtttaataacttccatttttaggacaaatgcagaggctataccagcctttgcaagaaagtataagacgagctgcacaacatgtcatgcagcttttccaaaacttaatgcctttggtgaggcgttcagaagaaacggataccagatcccagaggttgacgaacgctatgttaaggagaaacctgtcagcttgggcgctccggcgtggaaagaggtttggcctgaaggaatctggccaggcgagattcctggggcaatacccctttctttatttggaagtttgttttaccgatatgatgtaaactctcgggtaaagcatgattttacctttccaagtaccatagcgcttgaatcagctggaacactcggtgaagatgtatctttttttggcagtgtttctcttattagaagtggaaatgagttcggcggtttgtggcgttttttcctgaagtttaatgatacattcagcagctcgcttcctgaccgccttttgaatgtgactatcggtcaattcgaaccgtcagcagtcccggtctccaataatcgccgtttaacatatgcgccatatctcattaatacgtttacagtcgggaaaaacagctttaacttctcaaatcagcgaggcatagagatagatggcattatagagagccgcttagaatatgcagtgggcatagttaatggcaacggtacagggaagacagattccacaaccaactctgcagacaataatacgacaaaggacgcctgtcttaaggttggatataagttcggcggcattggcctggatggttctggccttatctcagacaaagaagatgtcctgccagaggtaaaggatgaaaaatcgctatatgttggcgtaataggttattcaggcaaaaataaagtggttgtttcaaacagcacgtatgacgataatttctataggtatggctttacctttgacttcaattataaaatttttaatctgtttggcgcggttattcagggggagcatagtaatccgagtggcaattttagtgatacctctgtaagttcctatttttctgaagccgaccttgccatttatccttggcttattgcagcggtgaggtatggagtagctgatatagaacatgagaaggaaaataaagatgaagtggttgtaagcttgttggcgctggttcgcgcaaatataaagcttactatcgaaggggcgctgcataccacaggtggcgagggggacctgggcatagttaaacttgattttgtcttttaagccaacattttcttatgccaattcgctctgcgagaatgccgtgtaaaccccgttagagaactatgtttcctaacgaccaaagatagtggcagcgcccaccgtaagacctctggtctcttacggggtgaactaccgtctctaaggaatgatttatactaccgtccgcctcaggcggaggagtcggaggctttctctaacggggtaaaatatacttgtagcgccatacaatgacaacacctcaatttttcaataatactttctcatagacctgggggaagcatctatgattttaatagacataagcaagaaactgtttaactcaaaaaatgcagttgcattttttgacaggagtggtaagtatgcgattgtgccgttgcagatgaaagcgcttctgcggttgagatatttagccgcatgcgcaatagtgcttattttgagctgcatatcgcatgccgagactattagaggaaccgtcaagctaaagagtgaagatgatgcggggaaagtagttatttatgtcgaaaaagctccagggggatttataccgccagagaagcatccggttatggaccagattgaactgatatttgttccgtatgttcttcctattctcataggaactactgttgattttcataatagcgatgatgtgctcaataatatctttactccgtcatgggcagggcacaaatttaatctcggcacctatcccaagggagtagtgagatcatttacatttgaccgtctcggagaggtagcattattatgcaacatccatcctgatatggagggctatatactggttttgcagaatccgtattttgctatccctgacaagcaggggaggtatgagataaaggatgcgcctccgggggtttatgatttcaaaatgtggtataagaggaaagtaagcccttcttatacggtgactgttgaaaaggggaaagatgctatagtggattttaaataagggtaacgggaggtaacgacgttgaaggtgacaataagggcaaaattaatgattgggtatatagcagtcctaatctttatgattatggtgagtatcttcgcatatatcacacttgttaatgtcaacaaaacagtaaatcaaatacttgtttctgcccataaatatgacacggttaacagcttgcggcagtctgttaaacaatttgttgatgtcagtgattcgttagtaagagggcagattcaggatgtgggttattatcgctctctaacgcttgatgtgaaaaaaaagcttttgtatgtccgtaaactgcgactcaaggagcatgaaaaggatttcttggaaaaagtagatgctgaatttgactctatacaaaatctaacagagcaatccttaaagtggacagcagctttgcggaacacaaatctcgttggcgtgctcaaggaattggacaaggccaagccagttataatcagcagtgttgatggactgtatgatgaggcatggcgctcactggataacgttactattatgactaacgataatatgaggaagggcctgcaacagattctgacattttcgataatcgctattttggcgggaataggaatatctatttatatttctcaaaagattactacgccaataagggcattatccatgtccgctgccagtgttgcgaaaggcgatttggataaaatggtggagaagtcgtctgcagatgaaatcggggaattagttgcttcgttcaatcagatgctggttgaattaaaaagctcaagggaacagattgaaaagtataacaaagagcttgagaccatggtggatgaaaggacagcagagctggaaaagacaaaggaataccttgaaaatattcttgaacattcgggagatatgattatcacaacaaccctctatgatgagatagtgcagtttaacagaggggctgaaaacatactgggctatgtcaaggaagaccttggcggaatcaagataggagatatttttgtcaataagagtgagtataagcggataagaaagaaggtaatcgaggacggagagatatccggttatgatgcgcagcttatgaagaaaagcggtgatatcatacgcgtaagtcttacgctctcgcgtcttgaggacaggactggcaatattatcggtcttgtaggcataggcaggaatataggcgaggataaaaaaagtatttaaatgaagatacataaaaaggggattcaacgcaggatagccatttttatatttgtcgtagggatgtttcctgtcatgataagcatacttttagtttatctgcagggggtgagggagcttagagtaagtgcgggtgaaaattttgcgcgagtagctaaagagatagcgagtaacgccgaaattgttatggagcaatctgttcacgatgttaaaagccttgctatttcaccggtcttaagaaatgcagcgatattagcaaatgaatcctatacaggtaaagataaggggtcaatagagaggcatatccagaaaattgatatgcagtggttgcagcatggagacaaggaaaaacgttttggggcgtatgtatccagtcgggctgcgcaatacttagcagaaataaagaaaaatacagagggatacgttgagctttttataaccgacacaaaaggggtattagttgcctccgcaggaaagacaaagtatttatactatggcagtgagaagtggtggcagacagcctataataaaggtaaaggagatatttacataagcgagatataccttgaccctgatatcaatcagttcctacaaagtgttgcagtgccaattatgtctgagaaagaaggaaaggtgctgggagttattcgtgcagttagtaccatagataagatttcgaagatagtgcagaaattcagaattggaaaaacaggccatgcaatgcttataaattccgttgggattatcctcctgtgtcctatattcccgcctcaggttcacagggttactgacgagctgatgaataatattaccacagcgccgttacttggctgggcggttgttaaggataatgcccatgaaggcattaatgctgttgcgggatttgcccctattgcaacgaccgatattgtaaagaatgcctttgatggaaacaaatggtatatctttgtcagtcaatcaccggatgaaagttatgcccctatttatgttttgctaaagagggttttttttctactgatattctctattttaatactctctgctatgggattctgggcggcaagaaagattgtgaagccgatagagacgcttcggcaaggggttgagataattggggaaggcaatttaaaccaccgtatccatattaagacacatgatgagatagaggagcttgcaaataaattcaatcagatggtggaaaaaattaacaagacacattctgaacttgaacagcgtatttcggagaggacagggtttttgagaaaaggctatcaagagatggaagtaatgagccgtcttaagtcagaatttctcacaaatgtatctcacgagctgaggacgccgcttaattccattttgggattttcagagctgctccacgataaggtttgtggagatctgaatgaaaagcaatctgaatatatagggtatatttatcggagcggtaaacatcttctcgatataataaatgacatccttgacctgtcaaagattgaggcaggaaagatggagataaaaccagaagattttttagtttccaatggaataaaagaggcatgcgctatagtgactcctttggctataaagaagaaaatacctattgatgttgaaatagcacctgatgtttatactatcgtggctgatataaggatgtttaagcagataatgtataatcttatcagcaatgctgtgaaatttacccaggaaggcggccatgtcagcattaaggttaagtccaacaactattatcttcaggtttcggtgattgatactggtgtaggtatcaaaagggaagatatgggcagcatattcaaagaatttaaacaggtctcaaccggagatcaggagggaactggtcttggtcttgtccttgtaaaaaggtatgtagaaatgcagggcggtagtatcagggttgagagcgaatttggcaaggggagtaacttcaccttcaggcttccgatgaatattacgaaaatataaagagcaggctatgggcaatgggctataggctatggggtttttcctattgcctatcgcctctaccctactgcctacttttaataggcatataaatttccatatggtcggagggaggcaggtgtcagcttcttaaaaccggatttcattatatcttcggttttaaggttaaagtctttgcaataatcctctaaatgttccttgagaaattttttgtcctcttcgtttaaatctgtaattccaacctcagcgccgcacccttttttatccacatcgcctcttataaatatcacgccgccatgcatgcctgtgccaaggtaatccccaaccaacggccttttattgtcgccatcaaggcccaggaggattaatacgccgcctgccatg

SEQ ID NO: 48 - DNA, 4671 bp, Deltaproteobacteria bacterium GWF2_42_12
cctctgtagaagatgtcataatacacaaaatatttgcaggacggccgagggatttagaggatgcaaggacaatcctcttaaaaaatcctgatattgacattagatatatacagggttggttaaaggagtttgacgcatcaattggcaataaggattttctgaaaacctttgaggacatattaaaaaccataaatcagtagaataggcttgtttttagtggtaaaaggaaggacagaaggtttttgagatatggtgatccccatcgtcataatcaaaggtaaataaaaatggaaagagaacttgttttaggcattgattacggcggcaaatataccggtcttgcagttgttgaccgaaggcataatcaggtattatatgcaaacagactgaagatgcgtgatgatgttgcaggtattctgaaagatcgtcgtaaacagcgtggtattcgtcgtaccgcacagaccaaaaaaaagcgtctgcgtgaactgaaaaactatctgaaaagcatcggctataatgaaagcaccgcaacctttgaaaccgtttatagcctggcacataaacgcggttatgattatgcagatatgccggaagaaaaaaccagcgaagaaattgaagcaatggatgtggaagaacgtaaacagtgggagaaagaaaaacaagaatgggaagaaaccaaacgcaatagccgtcatcgtaaagaagttgtgaaagacgttcataaagccatgattgaaggtcgtgcaaccgaagaacaaattaaacgtgttgaacgcatctttaacaaacagtatcgtccgaaacgttttaacaatcgcattctgaccaaatgcaaagtggaagattgtggtgttaatacaccgctgcgtaaaaatgttcgtgatctgctgattgaaaacatcgtgcgtttttttccgattgagcagagcgaaaaagataacctgaaagatgcagtgctggataaaaatcgtcgtgaagaagtgaaatccttcttccgcaaacacaaaaccgatgaacatattcgcaaacaggtgtatgatattgccgataataaactgagcggtcgtaccgttttttgcaaagaacatatcctggaacataccgaacacagcaaagaagaacgcaaagtttttcgtctggcaccgagcctgaaaaccaaaattgaaaatgttctggccgtgatcaaagatgaaatcctgccgaaattcaccgtgaataaagttgttatggaaagcaacaacttcgatattgcagcaaaaacccagggtaaaaaacgtctggcaaaagaagaatacggtaaaggtccgcgtgaaggtaaagaaacccgcaaagaagcactgctgcgcgaaaccgatggtcgttgtatttattgtggtaagagcattgatattagcaacgcccatgatgatcatatctttccgcgtaaagccggtggtctgaatatctttgccaatctggttgcatgttgtgccgtttgcaatgagaacaaaaaaggtcgcacaccgctggaaagcggtattagcccgaaaccggaaattattgccttcatgaaaaatgacctgaaaaaaaagatccttgaggacgcacgtaacattaacaccgtggatttcaacaaatatatgagccatgcaagcattggttggcgttatatgcgtgatcgcctgcgtgaaagcgcaggtaacaaaaaactgccgattgaacgtcagagcggtatttataccgcatattttcgtcgttggtggggcttcaaaaaagaacgtggtaataccctgcatcatgcactggatgcagttattctggcaagccgtaaaggttatagtgatgatggtctggttgatatgaccctgaaaccgaaatataacaaaggtggtggtttcgacagcgaaaaacatctgccggaaccgattgaatttaaacgtgataaaggtagcaaaggcagcgcactgcatgatcgtaatccgctgagctataaaaagggtattattacccgtcgttttatggtgaccgaaattgaatgtggtaaagaggatgacgttatcagcgaaacctatcgcgaaaaactgaaagaagccttcaaacgcttcgataccaaaaaaggcaaatgcctgaccgataaagaagcgaaagaagcaggtttttgcatcaagaaaaatgagctggtgatgagtctgaagtgtagcattaaaggcaccggtccgggtcagatgattcgtattaacaataacgtgttcaagaccaacgtgcataatgttggtgttgatgtgtatctggatgagaagggtaagaaaaaagcctacgaacgtaaaaatccgcgtctgagcaaacattttattgaaccgcctccgcagccgaatggtcgtgttagctttacactgaaacgtcgtgatatggttaccgttgaaggtgaagatgcaatctaccgtattaagaaactgggcacctctcctacgatagaagcagttgttggtagtgatggaaaaacaagaactgtctctgccaccaagctgacaaaagcaaacagtgctgaatagtttttaaaattaaatgtcctaacccacgatgaaggcgccatttctggcatagaggctcaggctggacaaacccaagcgtatatcccttcactgtataggcgttttacaattaaggctcttttaaaatttcgcaaaccccttaaggtttgtttcaaaaaatggtcttgcggaaattttataaattgtttttgaaaatccctttgaaattccagcaaaacaatagctgtttcacgccatattttcaaagagccttaactgtaacttcgatcttctcgaaggagatatgcgtgggcagatgtttcacgccatattttcaaagagccttaactgtaactgtgtgaagcggtgcaaggcgcatcccgcctttataagtttcacgccatattttcaaagagccttaactgtaactccttcttatacctgcggtaggccgcctgctcagaagtttcacgccatattttcaaagagccttaactgtaacgttcctcaatctgtgcggaaggccgccccgaaagttgtttcacgccatattttcaaagagccttaactgtaacgagaagtcatttaataaggccactgttaaaaagcttgtttcacgccatattttcaaagagccttaactgtaacttcataataccacactgaaacgaaactcatgctaatgatacttcggctgtcttgcctgtagattcattaataacaaggagtttaccctcatcctcttttttcaactgttcattggcattcagcaattctatgccatagaccgttccatcaggcgccaaatctatgttcagttcatcgctgattttcacagtctccacttgtgctggtttttcatgcaggcgaatataagctatgttgtatctcggatcatatgttaacttcatatatgttccaccgtaaggcttgcaatatggcgtatactgctttatagttacaccacataacctcaaaaatcaagaaaatattgaggaggtctatatggataatgaaacaatcaggtctattatccagatgcaggcaagaaacctatcggcttttttaagcggcaggagagaatataaggcgttcaggtttaagtggtaattagttcacgctgaaaaatgcccatctgctgcgttgccttcgtcgctgcgctgctcacatacctaaaagcgtatgctccgctgctcgctcctcggcgccttgcatctgtgcaatttttgagcgtgaactatataaaagcatttttcaactaattatgggagaacatactgtttatattttctatgacatagaagacgacggcatccgcaataaggtggcagagacctgcaaagactatgggcttcaaaggattcaatttagcggattttcaggcacgttgggcaaaaataagcgcgaagaactttttttaaagctttcctttctcattggcgataaagccggaaaactccttatgctccctgtttgtgaaaaagacatcaatgccaaaagggaactcatacaggaaaatagagatgattcatctcagggttagcgaccttaaacagtttgtctattgtcaaagggttgtgttctataactatgttatgcctgttgagaaaaaggcaacctttaagatggaatatggaaagatagcagaggataagatagacagattagaaaacagaaggaaacttaaggaatacggcttatccagcggcacgagagaatttcataagcaattatattctgagagatatgctttatccggcaaggtggatttgctaattaaaaccaaagattcttattatcctgtggattttaaatatacaacctctcagccgcacaaaaatcatctttatcagcttttgggttatgctataattttggaagatgtatataatcgcaatgttgataagggttttgtatacttaattccaaaagaggatgctgtggtttttgatttaaccggcaaagtcaaagaggaaacaaaaaatttattagacgatatccgaaaaatgatacactgccagcaaattccgcctccggtaaattctccggccaaatgtcttgactgcgaatatcgtaatttttgcggagatgtattataatccgaaatagtattatgcccctttcaaaagaagataaattgctcctcttaaaaatagtaagg

SEQ ID NO: 49 - DNA, 2077 bp, Unknown
  Soil microbial community from Rifle, Colorado
ggacgatgtcgctgatattctcaaaggccgcagagagcaaagaggtatcagaagaaccgctcaaaccaagaagaaacgtttgagagaactcagaaactatctaaaaagtattggatataacgaatccacagcaacatttaaaacggtttacagccttgcccataagagaggatacgattatgctgatatgcctgaagaaaagacttctgaaaagatagaggcaatggatgaggaagaacggaaacagtgggaaaaagagaaacaggaatgggaagagacaaaaagaaattcaaggcacagggaagaagttttaaaagatgtccgtaaagcaatgactgaaggaagagcagccgaggagcagataaaaagggttgaaagtatttttaataaacagtacaggccaaagagattcaacaacagaattttgaccaagtgcaaggtctgcggaaaaaatacgccattaagaagaaaagtgagggagttgcttcttgaaaatatagttcgcttcctgccgttagaaagcgaaatgaaagagactcttaaacagacaattcttgaaggacagcaggaaaatatcaataaaatattcaggaaattgaaatttaatcagaaagactggccgggcaagaatcttacggacatagcaaagaacaaactgccgggcagactgcctttttgcaaggaacatttctccgagaatgagaagttcaccatgattgaaaaatcaactttccgtcttgcaccatccttaaaaacaaaaatagagaatgtccttgctgttattaaagacgagatattgcccaaatttactatcaataaggtggtaatggagagcaacaattttgatatagccgccaaaacacagggaaaaaagcgattggcaaaggaagaatatggcaaagggcccagggaaggcaaagagaccagaaaggaagcccttcttcgagaaacgaatggtagatgtatctactgtggaaagtctattgacatatccaatgcgcatgatgaccatatatttccccgaaagtccgggggcttaaatatctttgcaaatttagtagcatgttgcgctgtatgcaatgagaataaaaaagggagaactccgttagaaagtggaatcctgcctaaacccgacgttatacgttttatggaggatgaatttaacagacttaaaattaagatgtggaagatcaggcgaaagcagtcattgacaactgaagagattatggataaggcaaagcgcaataagcttgaactgaaactaaaaatccttgaagatgcgcagcaggtcaatgaactcgatttcaacaagtatatgtctcatgcctctatcggctggagatatatgagggacagactgagagagtctgccggcaataaaaagttgcctatagagaggcaaagcggcatatacactgcatatttcagaagatggtggggatttaaaaaggaaagaggcaacacccttcatcatgcccttgatgctgtaatccttgcttcaaggaaagggtatagtgatgatggattggtggatatgacccttaagccaaaatataacaaaggtggagagtttgaccctgaaaaacacattcctgaaccgatagaatttaaaagggataaaggcagcaagggctctgcattgcatgatagaaacccactgtcttataagaagggcattattaccagaagatttatggttacagagattgagtgcggtaaagaagacgatgttatttccgagacatatcgtgaaaagctaaaagaggcttttaaattatttgatacgaaaaaagggaaatgtttaacagacaaggaggcaaaggaagcaggtttttgcattaagaagcatgaattggttatgagccttaaatgcagcattaaaggaaccgggccagggcagatgataagaatcaataataatgtttttaagacgaatgtccataatgttggcgtggatgtttatcttgatgaaaaaggtaagaaaaaggcatatgagcgaaagaatccgagactgtcaaaacacttcatagaaccaccaccgcagcctaatgggagagtttcatttaccttaaaacga

SEQ ID NO: 50 - DNA, 4589 bp, Unknown
  Soil microbial community from Rifle, Colorado
gcttgcaatgaaatttaatgctacggatactaacacagaactattgaacattacaaaccttaaaccccttaagcccgggccgtacattttccggcaaattgcggccttgcacagcggatataaaacgaaaaaactttactgcctttcttgctttcaatcatggaagttgttataaagatggttgcgaagatggcaggaatgaaagaaggtagcaggcagggaagggtgtggtatgatggctgcaaatttgacaatttctaattttaatagaaccgttgtcccattaaccatcttgctaaaaaccgatgggagaggaaagagagtgtttgaggtttagcatgaggaggttttatggaaagagaaattgttttaggcattgattacggtggcaaatataccggtcttgcggttgttgaccgaaggcataatcaggtattatatgcaaacagagtgaagatgcgtgatgatgttgccgatattctgaaaggtcgtcgtgaacagcgtggtattcgtcgtaccgcacagaccaaaaaaaagcgtctgcgtgaactgcgtaattatctgaaaagcattggctataatgaaagcaccgcaacctttaaaaccgtttatagcctggcacataaacgcggttatgattatgcagatatgccggaagaaaaaaccagcgaaaaaattgaagccatggatgaagaggaacgtaaacagtgggaaaaagaaaaacaagagtgggaagaaaccaaacgcaatagccgtcatcgtgaagaggttctgaaagatgttcgtaaagcaatgaccgaaggtcgtgcagccgaagaacaaattaaacgtgttgaaagcatctttaacaagcagtatcgtccgaaacgttttaacaatcgcattctgaccaaatgcaaagtgtgcggtaaaaatacaccgctgcgtcgtaaagtgcgcgaactgctgctggaaaatattgttcgttttctgccgctggaaagcgaaatgaaagaaaccctgaaacagaccattctggaaggtcagcaagaaaacatcaacaaaatcttccgcaagctgaagttcaaccagaaagattggcctggtaaaaacctgaccgatattgccaaaaacaaactgcctggtcgtctgccgttttgcaaagaacattttagcgagaacgagaaattcaccatgatcgaaaaaagcacctttcgtctggcaccgagcctgaaaaccaaaattgaaaatgtgctggccgtgatcaaagatgaaattctgccgaaattcacgatcaacaaagttgtgatggaaagcaacaacttcgacattgcagcaaaaacccagggtaaaaaacgtctggcaaaagaagaatacggtaaaggtccgcgtgaaggtaaagaaacacgtaaagaagcactgctgcgcgaaaccaatggtcgttgtatttattgtggtaagagcatcgatattagcaacgcccatgatgatcatatctttccgcgtaaaagcggtggtctgaacatttttgcaaatctggttgcatgttgtgccgtgtgcaatgaaaacaaaaaaggtcgcacaccgctggaatcaggtatcctgccgaaaccggatgttattcgttttatggaagatgagtttaaccgcctgaaaatcaagatgtggaaaattcgtcgcaaacagagcctgaccaccgaagaaatcatggataaagccaaacgtaacaagctggaactgaaactgaaaatcctggaagatgcacagcaggttaatgaactggatttcaacaaatatatgagccatgcaagcattggttggcgttatatgcgtgatcgcctgcgtgaaagcgcaggtaacaaaaaactgccgattgaacgtcagagcggtatttataccgcctattttcgtcgttggtggggcttcaaaaaagaacgtggtaataccctgcatcatgcactggatgcagttattctggcaagccgtaaaggttatagtgatgatggtctggttgatatgacactgaaaccgaaatataacaaaggtggcgaatttgacccggaaaaacatattccggaaccgattgaatttaaacgcgataaaggtagcaaaggtagtgcactgcatgatcgtaatccgctgagctataaaaagggtattattacccgtcgctttatggtgaccgaaattgaatgtggtaaagaggatgacgttatcagcgaaacctatcgcgaaaaactgaaagaagccttcaaactgttcgacaccaaaaaaggcaaatgcctgacagataaagaagccaaagaagcgggtttctgcatcaaaaaacatgaactggttatgtccctgaaatgcagcattaaaggcaccggtccgggtcagatgattcgtattaacaataacgtgttcaagaccaacgtgcataatgttggtgttgatgtgtatctggatgagaagggtaagaaaaaagcctacgaacgtaaaaatccgcgtctgagcaaacattttattgaaccgcctccgcagccgaatggtcgtgttagctttacgctgaaacgtagagatatggttacagttgaaggtgaagacgcaatctatagaataaaaaaactcggaacatctcctacaatagaagcagttgttggtagtgatggaaaaacaagaactgtctctgccaccaagctgacaaaagcaaacaatgctgaatagtttttaaaattaaatgtcctaacccacgatgaaggcgccatttctggcatagaggctcaggctggacaaacccaagcgtatatatcttctcgtataggcgttttacaattaaagctcttttgaatttcgcaaacctctatgggtttctttcaaaaaatatcttttcggaaattttgcgatttgcttctgaaaaaccctttgaaactccagcaaaacaaaagcggtttcacgccatattttcaaagagccttaactgtaacaaagtaagagtgccggttgatttgaaattcgtgccaaagtttcacgccatattttcaaagagccttaactgtaactatacatattatattcaatttcctgcaaccattctgtttcacgccatattttcaaagagccttaactgtaacctttgattgtctcaatcagggtatccaatttaagggtttcacgccatattttcaaagagccttaactgtaacaataagaagccgtcattgctgcaagcgtgccgtcgccgtttcacgccatattttcaaagagccttaactgtaacatgtattgcctgcaacattctggatgattccaaattcgtttcacgccatattttcaaagagccttaactgtaacgagaagtcatttaataaggccactgttaaaaagcgtttcacgccatattttcaaagagccttaactgtaacttcataataccatactaaaacgaaactcatgctaatgatacttcggctgtcttgcctgtagattcattaataacaaggagtttaccctcatcctcttttttcaactgttcattggcattcagcaattctatgccatagaccgttccatcaggcgccaaatctatgttcatttcatcgctgattttcacagtctcgacttgcgctggtttttcatgcaggcgaatataagctatgttgtatctcggatcatatgttaacttcatactatacccaaactctgccgcgcctctttccgccaatcgttcctatgcgtgtatatgaaattttaccatatacggcttctatcaatatgtcggaactgttgcaatcgcaagacttgcaatatgatttgcactgctttatagttacaccacataacctcaaaaatcaagaaaatattaaggagggcatatgggtaatgataattcaaaaatcctccgcattcatccggaataccaaccagcagaaaaactcccgcctgcacagttcaatctctttacaggagaagcagagccaattccacaactgattgacaaaagtattgatgacgaaatattcattgacaatgggatttcgcttgtgcagagcggagattcctcacaaatagttctgtcaggttttggcatctttttaagcaaaaagagcgaacagcttatcgtaaagaagagcgacaaggtcatttatgaattttccctttgacaaacttttttctacctgcaataattggccttgccccaaaaatagacattgatttttggggaaactgacaagttgcctgccagacgaggcggcggccatttttgtgacgcaaccgtatgctcaccgatacggtgaggaacaaaaattggccgataacgaagtatggcaggtaaatcgccagtttgtaaaatagcgggggttttaatcgctattttacggcttgtttttaaaattctattgtattgcaagcttatagatgatctcagaaaacaaaagactcaaaaacacacccctgcatcaaacccataaagaactcaatgcgaggctggttccttttgcaggatgggaaatgcctgtgcagtacatgggcgttatagaggaacatcttaatgtccgcaaggcctgcgggctttttgatgtaagccacatgggtgagatagagatatccggccctaaggcccttga

SEQ ID NO: 51 - DNA, 2032 bp, Nitrospirae bacterium RBG_13_39_12
cacagacattcttgcaggtcgcagagaacaaagaagtttaagaagaacactacaaactaagaaaaaacgtttaagagagctcaagaactatcttgaaagtattggtggtatatatgaggaatcaagtgggacatttacaattgaaccttttagaacagtttacagtcttgcccataagaggggttatgattatgccgacctccctgaagaaaaaacttctgaagagatagaggcaatggatgctaaagaacgaaaacagtgggaaaaagagaaaaaagaattggaggaaacgcaaaggaattcaagacatagagacgaagttcttagagatgtacgcaacgtaatgactgaaggtaatctatcagaagagcagataataaaggttgaatctattttcaataagcagtaccgccataaaaggtttaataaccgaatcctgactaagtgcaaagtctgcggtaaaaataccccattaagaataaatgttcgggaattgctacttgaaaatattgtgcgttatttgcctctccagaataaggaaagagaattgcttaaattgactatcttgaaagggcatcagcaggatataaatgaaatctttaagcacttccgaaaagtttacaagataactctaaaccaaaaagattggccagggaagaatcttattgatatagcacggaatcaattgcggggcagattacttttctgtaaagtacattttcccgaaaatgaaaaatatgtttctattgaaaaaaagaccttcaggcttgcaccatccttaaaaacaaagatagaaaatgttctgtcggttatcaaagatgacattttgcctaatttcactttaaacaacgttgttatggaaagtaataactttgatatagcagctaagacaaagggaaagaaacgattacttaaagaagaatacagtaaaggacacagagagagtggagaaaccagaaaagaagctcttcttagagaaacagatagccgatgtatttactgtgggaaaggaatagatttatctaatgctcatgaagaccatatttttccgagaaaggctggaggtattaatatctttggcaatttagtcgcatgttgcagcgtatgcaatgaggaaaaaagaggaaggacaccattagaaagtggcattttgccaaaaccagaaattgtatctttcataaccaatgacttgaagaaaaagattcttgaagatgcacaatatattaatactttagatttcaacaaatacatgtctcatgcctctattggatggcgacatatgagagataggcttagagaattaactggcaataaagaattacttataaagcgacaaagtggtatctatactgcatactttagaaagtggtggggatttatcaaagaaaggggcaaccacggacatcatgcacttgatgctgtaattcttgcttcaaagaagagctatgcagaagatggcaaagtagatatgactattaaaccatgtggcgaagatggcaaagaatttgacatagaaagacacctttctgagatgaaagagttcagaagagataaagggggtaaaagtgctcctcttcacgataggaatcctttatcttttaagaacgatattataactcgcagatttatggtaactgaaattgagtgcgggaaagaagccgttattatttcggaggaatatcgtaaaaaactaactgaggcttttaaacgctttggtattgctaaagggaaatatttgacagatgaacaggcaaaggatgctggattctatttaaggaaaaatggcgaaggggtgatgagtcttaaatgcgaggtaaaaggaactggctataatcagatgattagaataaaaaataatatttttaaaacaaatgttcataatgttggcgtggcagtttttcttgatgaaaaaggcaaaaaaagagcttgtgaactcaagaacccaagactgtcaaagcattttgtaaagccagcagagcaggttaaaggaaaagttatattcatcttgaaacgtggtaatatggttacagtt

SEQ ID NO: 52 - DNA, 5152 bp, Nitrospirae bacterium RBG_13_39_12
catttgttaagattcagaactgacattgaaaatgctaaaaccttcaaagaagcctcagattcttttttaactttattaaaaccaatatctaaggagaaacctgctgattttattcaagagacaaaaaagcaaaggatgacaagggaagatgcctacggaggatatgaaagaaagagtgataaatacaagggcaaaaaaagaggcgatatttggaatgttattaaaaaggcagaagatatactgactggtaagaagataagtgagtgggagcgcatcggaataggtgaactcgaatggttcaagcttttttcaaaactctcaagaggaattgctaaaggaattgaacttcacgacttagaagcttacacgggatacagtacaaaggcgatccataaaatgatagatgaacagtgtgtattaaaggatgtgttggatatctttaataaaaagattaaaacagcattagcaaagcaaaaacctgatgacaggactttcttaaacacgatcaaaggagtatggggtaaaaaggcagcattggtcttagagaattgtacttatgaaggggaacgaggccgtaaagtctctaaggagactataaaggagaggcataaaaggataagagaagttatttacagtgggaaaattgaaaaggttaattcagggacttactacagtagagactttagagtatttctatgcgtaatggctcgtgttgtttatcacctcttgtttgaaattccctttttaggcgacaaattgaaaagagaagcaatatcagagtttagaaattttgcaaaaaaatatgatataaaactctctaaattatctgacttccaatttatagaaagcaaacccacatcaaaaggcttacccgacataaagcctcataaatagccattcttcctaatagcacgaaatagattcaattccgttcctttcagatcaatcagaaatccgacataataccaatatatgtaaaaaaaagatttttgttccaattttgttcccgaatccgaaaaaaggaggtaaaagaataatgggaaaacacacctttgaagatacttcaatagtagcattcttatctctcaaaaattacaaggtaacaccacagagaacctatgacggaaaagttgtctttattgtagaaggcaaggatattaacagggcattgcaggagctttatggcaattctcaagtaggtgttttggattttattaagactttaaaagccttacgctcaagtatctttgcactcaaagcagggggtgaaagatgaaatctatacttgatcagaatttaatcactgtatattcattcccttttgatatatttccatcaaagctattgaatgtcattaatgtactaagtgaagccttacatgtggagcctgaaattgtagcaagtgcaatgcttaccataataagtgggacaataggcaacactgtaagaatatcccctaaacatggttttgaagtggcaccctttatatggctgattatcattgccttaagtggttatggtaaatcccctgtgattcaaacccttctaaagcacatcaatgaattgcaggcaaaggcttataacgaataccaaaagcagtttcaagaatatgaaagaagacttagaaaggctaaacaagatgaaagcatagatataccagagaagccaaaattaaaacatcatgttgtatcagattgtacggtggaggcattggctaatgtgtttgagaatgatagcagaggggttattagctatcaggatgaaatagcaagtttaattttaggtcttgaccagtacaaagtcaaagggaacgatagacagcattatttggaactatttaactgtgatagctggaagatagacagaaaatcaggtgtcaagtttatccacaacacaggtacttctattattggtgggatacagcctaaagttatgcctgacgtcttcaaagtaaattcttttgatgatgggtttctaccccgtttcttattgctcaatgctgaaaacagacccatgaagtttagcagacaagcaattattgaagagattatatcttactggagagacctattaaattggtgctatgccattccattagaacatgatgatgatggattcataaaaccaaaagttttaaccctgagtagcaaagccttagacatatgggaacaattctataacgattacggcgataagatgcctttcctttcagaaagggcaagggtctttatcccgaaacttatagcctatcacagtcttaagtttgctggagttttacatgtcatagaggcattcagcaaaggtataacatccataaacaacttaatcgaggatgagacaatacatcacgctattgaattaataaaattctatgctggacagactattaaggcattgaaactgtatgagaaaccagaagacactctaaatgaatttcagaagagactcattgaaacactacgtagcttgaagggagaggttaaaaacgggaaattacagttatcaagaattgttgagctattcaatcatggcgtacctgaaggtgtgagtcatactccagaaggaattagcaatatgttggctgatttggggttaactactgaaaaaagcacgggcaattattcatatcttttatgggaatctgagaagatacaaaaacttttttctaaaacaaccgtaactactgtaactactgtaactactgtaactgataaatcaaattcagaaaacaacaaagttactgaagttacggatgttaccgttaattctgatgaagaagttatagaccttgaacatgaggagtttgaaatagtcaaatgagatacaaagtcttagaaagtttcaagattaaaacgttaaaaaaggaaatggaactgcaagcagggcaggttgtcactcttcctcatgacaaggctatcattctaatcagtgaaggtaagattactccctttgatgatacagcttatagggtctactcggagaatctacatgcttacctttgggttgtagaagatgataaggatattgaccgtctcagagaacaaggcataaaagaagctatctacacaagacaggaaatagagaaactaaaagatattgataaagactctctcaaggtaatccacagagttaaagaggtctttgagtcatcaaaggtcgtggagataaagggctgtgaagactgagcttgaaacacaggagattgaggcgatagccgataaggttatcgagaaaatgaaaccgttgcttgctggtaatggtaagtcagaggatgatgttatctttgatgtagaggggttaactcaatatttgaaggtatctaagcaatggatttacgaaagaacacacctgaaaaagatagcacacttaaaaatagatgggcaattgagatttaggaaaaaggatattgataaatggcttagctcttataatatccccgctataaacacaccttgaaggattttaaaggctataaagtaagaggaggccataagattgtatgtccgataacctatataagaaacccttaataataaacaatctacaaaccactgtgctttaaattgtgcactaaaaataattcttgactttctaacaatgttagtatatgatttagatatgcaaacctggacacctgaagaaatagagcaattcagaaaagacaacaagctaagcagaagggctttaggtgagcttttaggtgttacaggcaattgcatatatcaatgggaaagggggttgagagagccgagtaaaacaacaaaaatacttttatcaaggattgagcaagaactaaaaggaaagaaaaaacaaaaaggaaaggtaggtgaaaagaagcatggcaaaaagtaagcatcgggggatattcctgagaaaccgcatttactggattagatatgcagggcttgatggtaagatggtttatgaatcctcaggcaacgaaaagtttaaagatgctgaagccctgcttatccagcagaagcaatctatcaaagaaggcaagcaacctgagattaggaaaatctccaattacacattcaaagaactttctgagaaataccaatcatggattaacggcagacagaaatcagccagaacaaaaggctatctcatcaaacagttggttgaaaggttcggtaactttccgattaggagattcaatacggtgattgtggaacaattacagacagaccttatgaatagaggattaaagaatagtagctgtaataaggtcttgaatgttctcaaacatatgtttacaaaagctgtggaatgggagatggtagagtctgaaacattaaagcgtaccagaaaagtaaagctattaagagacgatgggaaaaggttaaggtatctctctacagaggagtgtcaagccttaatcaatgcctgtgataatcacctgaagcctattgtagtaacagcagtgaatacaggcatgagaaaaggtgaaatactctctcttaaatgggataatgtagaccttagacatggcttcattctgttagatgtaacaaagaatggagagagaagggaaatacctattaacgatactctcaggataaccttacagggtattacaaggcggttagatattccttatgtattctatgaccaaaatatagggaaagcctatcaggatgttaagagaagctttaaaacagcattgagaagaacagggataagagattttcatttccatgacctgagacatacctttgcatctcatcttgttatggcaggggtagatttaaccacagtgtcaaggctgttagggcataaaactttaactatgaccttgaggtatgctcatcttgcacctgctcacatggttaaagctatggatatcttggacaataccttaaatggtaagtttagctcagtaggtgaacgtttgcatgatgcaaactatacaaaaactatacaatcggcagaggcaaggtaagatgaaatctcctaagtgcttgaaaaatatggtaggcacgaggggtatcgaacccctgacctcttccgtgtcaaggaagcgctctaccactgagctacgcgcctgtaagactttataaaataacattttacccattactcctgtcaaccttgagtttatatcttttagtctcttttgacagttaatcatctctttttatatactttc

SEQ ID NO: 53 - DNA, 5463 bp, Nitrospirae bacterium RBG_13_39_12
tggttaaaactacctcatgtttattataacaaatttgtttattttgtgttgaagtatccactgcacaggaagaaaaggggaggtgttaatgctcactaattaggtaactttttggagtttagcagttcatcatatttaccatcgagataagtgcttgaaaaatatggtaggcacgaggggtatcgaacccctgacctcttccgtgtcaaggagagtctgtcgtatgtcgaggttttacgaatctttataaacctttgtaaatttttgttgcaaaggatttggggggagaagggttttatatccttttcccttcttttttgactgtctactatacaaaaactatacaatggcattaaacaatacttatttttcagacccccctacatcccctttttcagaagaggggtgggggttaagtgcttaccccctttctcgcatatgaaatcagttttatgggaaaggctaaaattgacctaatagacgctgtctaatttaaaggttcactttgaggatttcacctacaactattcatcttcagcaagttcaaagagttaactatttccgagagaaattaacaactattcttttgtaaactcatcaataaatttatcaacattatgtgtcttacgctgtatcaacttaaataaacgccaaagttcccctggggctgataactcttcaaagtcaaattcatattccaagataaaatctcttgacgctaatgcaagctgtggacttgaacttccctccttaatataaattactatagatttgttaccaaattcacaataataaccatctttcagtgtcgagatatcatcacgaatgaactgataaataatatttggagataggtcattaataagactattccaatctaatttatttgcttttgttttttcaattaagctccgaagaaaggtcatttctttcataataaaaattcctcttttttagaaagtttgccaagcaaaatagctaccttatctgcgactgctattgtatcaatattgtctaaatcaccatctaataatcgttttagtttctccagttcccttttatgttctttcccaaattgaaattcttccaatgtgcttatgagtctttttaaatcggaaatctgttttctatcaaaaatattatcttcatttataagccgtagacacccatctaactctgcataaattttatttttttttgaaccccattgtttataatcaatgtattttattaaagttttctttaccccatgagcatatattaataatataatgctaactactaagctaagaacacttgcccaattagctatatagtcaaacatctccattccattagttaacgcattcaatatatatgacagttaggaaatagacacttgattaccgatacaaatctacaatttattcataattcctgtcaatgttaacttcaggaagaagcaaaccggaaacttttcttcctgaaggtgacaccctgaataattcgcaaaatgtgagtagtactcttggtattaaacagttgttaaggtggaaacaatgcgaagtatgtgtagaacaaaacaaggataatgttttataagaaaagaagtgcgatatcatccagatgttaatctttttcgccttttacttgctgccatgaattttgattctaaaaaatgacttttctaacaaataattccttgtatttttaattggaaagttgtaaaaaaatcttatactttaacttgacttattatggaaaaagaacttgttttaggtattgattacggtggcaaatatactggtcttgcggttgttaaccagaaaaataatcaagtactttatgcacgaacagtaaagatgcgtgatgatgttaccgatattctggcaggtcgtcgtgaacagcgtagcctgcgtcgtaccctgcagaccaaaaaaaagcgtctgcgtgaactgaaaaactacctggaaagcattggtggcatttatgaagaaagcagcggcacctttaccattgaaccgtttcgtaccgtttatagcctggcacataaacgcggttatgattatgccgatctgccggaagaaaaaaccagcgaagaaattgaagcaatggatgccaaagaacgtaaacagtgggagaaagaaaaaaaagagctggaagaaacccagcgtaatagccgtcatcgtgatgaagttctgcgtgatgttcgtaatgttatgaccgaaggtaatctgagcgaagaacaaatcattaaggtcgagagcatcttcaacaaacagtatcgtcataaacgcttcaacaatcgcattctgaccaaatgtaaagtgtgcggtaaaaatacaccgctgcgtattaatgttcgtgaactgctgctggaaaatattgttcgttatctgccgctgcagaataaagaacgcgaactgctgaaactgaccattctgaaaggtcatcagcaggatattaacgagatctttaaacacttccgcaaagtgtacaaaatcaccctgaatcagaaagattggcctggcaaaaacctgattgatattgcacgtaatcagctgcgtggtcgtctgctgttttgtaaagttcattttcccgagaacgagaagtatgtgagcattgagaaaaaaacctttcgtctggcaccgagcctgaaaaccaaaattgaaaatgttctgagcgtgatcaaagatgacattctgccgaactttaccctgaataatgttgtgatggaaagcaacaacttcgacattgcagcaaaaaccaaaggtaaaaaacgcctgctgaaagaagaatatagcaaaggccatcgtgaaagcggtgaaacccgtaaagaagcactgctgcgcgaaaccgatagccgttgtatttattgcggtaaaggtattgatctgagcaacgcacatgaagatcatatctttccgcgtaaagccggtggcattaacatttttggtaatctggttgcatgttgcagcgtgtgcaatgaagaaaaacgtggtcgcacaccgctggaaagcggtatcctgccgaaaccggaaattgttagctttattaccaacgatctgaaaaagaaaatccttgaggatgcccagtatatcaacaccctggatttcaacaaatatatgagccatgccagcattggttggcgtcatatgcgtgatcgcctgcgcgaactgaccggtaacaaagagctgctgattaaacgtcagagcggtatttataccgcctattttcgtaaatggtggggctttatcaaagaacggggtaatcatggtcatcatgcactggatgcagttattctggccagcaaaaaaagctatgcagaagatggtaaagtcgatatgaccattaaaccgtgtggtgaagatggcaaagaatttgatattgaacgccacctgagcgaaatgaaagaatttcgtcgtgataaaggtggtaaaagcgcaccgctgcatgatcgtaatccgctgagctttaaaaacgatataatcacccgtcgttttatggtgaccgaaattgaatgcggcaaagaagcagtgattatctctgaagagtatcgcaaaaaactgaccgaagcctttaaacgttttggtattgccaaaggcaaatatctgaccgatgaacaggcaaaagatgccggtttttatctgcgtaaaaatggtgaaggtgttatgtccctgaaatgcgaagttaaaggcaccggttataatcagatgatccgcatcaaaaacaacatcttcaaaaccaacgtgcataatgttggtgttgccgtttttctggatgagaaaggcaaaaagcgtgcatgtgagctgaaaaatccgcgtctgagcaaacattttgttaaaccggcagaacaggtgaaaggtaaggtgatttttatcctgaaacgcggtaatatggttaccgtggaaggtgaagaaatgatatatagggttaagaaacttggtacttctcctgttatagaagccattgtcggtagtgatggaaaaacaaggactgtatcagctactaaactcttgaaaataaaccatactaaaaaggtttaattaaattgtcctaacccacaatgaaggtgccatttctggcgtagaggtttaggctggacaaacccaaacgtacataccctcaccgtataggcgttttacaattaaggctcttttaattttcgcaaaccattaaggatttatttcaaaaaacaacttttctaaacttttatgaattatcactgacaaaacctttgaattaaaatgaaaataattccgttacagtcgttgttttcaaagagccttaactgtaaccccatatatcagttataaacactatattctgccatgttacagtcgttattttcaaagagccttaactgtaacctaccagtgagccgattcataaggcgaacagtaaggcgttacagtcgttgttttcaaagagccttaactgtaacgagaagtcatttaataaggccactgttaaaaagctttttacagtcgttgttttcaaagagccttaactgaatttgcaaacataattaaagtgtaatatgattacaccatattgcctaaaaatcaatagtattataatagaataaactattatataataagtcaatataaatcctaaattgtatagtattttcaaactgttatggggttcaaagaattatgagtttattaaaatatggctatttgggcatgttttttcggggaaagggggtctcacgtacacgaaaagctaatttttcagaaaagcgtaaaagatagctttccccataactgagacaaaagagagattgatatacttttggatttgtgcatataatggcaaaagtgggggattcataactatgatacatgttgctccatctgattgcagatagttaaatgataacttctaatacatgctatccatttggtgggaaagtcgcaacagtcgaaacaccatcatttaaatgcatgattagtacagccaaagaaggcgttttcaagaaacagtccgtagtttttcttgatattaatatgggcatggctaatcgccgacctgtcgctaagctgcattacaatgacatactcactaaagaacaacgtttgcaatttcacgatatcatagtaagcgtgatagatgaatccggcatgaatggactatctctctatgatacactcagggacttgtttcaaggacttcgcaaagagggcattggttctgggttttttacagatacccattgatgacggctgagaagtgaaaagggaataacttaaaacaattgtggagacatacttttcaaaaaatccccttccctttacctttgaaatagtctgttgagataaagtgaaagaagaaaaaataagggaagacgttagggattatttaaactcttttctcattgaaggtggttattgttttaatgaccctaaaaaattgtgtttagccattaccagtttgtcgaacgacatatatatcttcagcaaaaaagatttccttgtg

SEQ ID NO: 54 - DNA, 9183 bp, Nitrospirae bacterium GWA2_46_11
  misc_feature (145)..(264): n is a, c, g, or t
gcttattcaggaacttaacggcaacacgacaggcgataggcaagaggctataggcaatgggcgaaagacaaaaggcatcggcgcattgcaggcaaaacttaaagaggcgagggaatccgagtataaggcaaaagaagacgccttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntttagacgcccttaaaaaggaaagggagaagggaatacttacggacatagtggacagggaaaagagcatcagcgccatagaaggagaggcgataaaggcaaagaagaaatacgaactcgaaaggctttgctctcctgttgacggcactgttcacggacttgcctcatacactgtgggaggagtggtaactcctgcccaacccttggtcacggtagtgcctgaagggacgcctcttataatagaagcgacggcattaaataaagacataggatttttaaaagtcgggcaggaggcggaggtaaagttagatacatttccgtttcagaagtacggcaccgtaaaaggtaaggtaacggccataagcccggatgccttcgaggatgaaaagttaggaccggtgtataagataaaggtaactatcgaaaaattgaagatctatgcggacgacaggtatatacccgtagcgcccggaatgagcgtgtcggtagaagtgaagacaaataaaaggcggataatagagtttttcctttcgccgatcgtcaaatacgcgaaggaaagcctgactttgaggtaaaaacgacagacttagggcgataggcgataggtaatgacggatttaaattggcagcttaggactaatgtctaaaagtgagtctgaccttaagcggatgataactaaggagtataaaatcgaagatataataaaaccgaaagggaggccgaagaaagatgaagacaagaataaatagaaccgtcccgttttttgtatcgaggagtattctctcccccagggagaaagagattttggagcgcatgagagacggcaaaaccgacctcgaaatatccgccatttataccatcagcgaacacactgtaaagtttcatataaagcatatcctgcgtaaactgcaagcttcaaataggacacacgccgtaggcatcgccatgcagaaacgcttgctcgattaattgacatcgaatctaccccctactcaaatgagtagcttacattggggaaggggaatgtggtatataaaggcctgcatgtcttgcagccgaacttatagggaaggtatgaacgaattcagtacggcgtaaaagagtaaaagattgtggaaattactgctcaacagaaatgcaaaaattcctggaagactattccgacagattgcctcgactgtcaatgtccgctccgagccgattgcagaggaggatgcacagctgcatcagatgaggtatatggttcacaatatcggattgatccaatctgtgagttatcttgttaaaaaaattaggttggtttttcaacgataaggaaagagcatgccttattgagatcgaataacgtgaggcaagatataatcttgcagattactctaagtgggaggattgtatgcctgacatgaaagattttttaaatccgaaatcgatgttaacgcccggcattgcaggcggattaacagccagtgtttctttaacattggcatctgcatttgcaataagcttcaaatggtcggcacttggcatcagttttttgctggggttgcttataatgatttccatgcggaactctattcccttattgcagcgatttatatattgcatcctaaattcgctgattgttttttctatggcgttcggcgccggtaagggcattgattcgcagcctcagctcgatcctaagattattgacaaaattgtgaatacgcaattatcagtagttgagcctcaacccccttcggttttagcgttatccaatgcatatgcgcaacaatatcccattaaatcagataagcctaaaaaaagcactgctccttccggaaccgacaaaaaagggacaggcagcaatgaatcaaaggaaaataatctgaccaagaaagagatggaacagttaaaaacgtatattcaacaacaacagaattatgataagcgatggtcgtggtaagagaaaagtgaaattttaagaacagaaaatggggaggagaaaatgtgtaaagataagtcagtgagtcatttaaacgagttaggatggaatgtggtaagattacctaaagaaaatattaatccgcttttaactctttccaaatccaatagctatcttgaaactttaggagaaatctctgatttcgtgatagaagatcaacccaagcccccagaaattgtccaagaccaatcagtagcagaaatttcaggtttggagactgacaaatttgaattgggaataggactaaagtttttagaaaaattcctatctttagtgggagtagcgggattaggattagaagcaagctttaagaatgcagaatccattcaatttgcctatcagaatgtactgacagattttgtctatccagtgaagattggaaagtacttattgagtgtcagtcctgatgtcagtagcccctttatggagcatattaatgaagagggagaggcttatattattacagacacgcttagatctaacacattcggtattgttgcctatgatgaaaaaggtgtaaaaattgatttggatatttctgcattaaagcaacttcttagcgctactccacatattgaagtatctaaagatgagaagaaagttgtttcttttaagggggataaatttctgcgttttgcctttaaggcaatcggcgtatgggtggagatacgggacggcaaagcgagatttaaactgaataagccggaaggacccattgcaccaatgaaggctttaccgtccactttaattagtcccaatgaaccaacacctgtaattttcggtatgaacaccctaatcagactaaaatgaatcataataactaaaaaggagataaaacatgtcgaatcaagaatcgatcaacagaggtcttgacagggcatataatgagttgaaacctgaagatacgataaaatgcgatatagagtctgtgcgtatcgttatcttttcagatcatcacaggggagttcgcgatggagctgacgacttccagcggtgtgaaggggcatattgtgccgcactcggatattatttagagagaggttataacctcatagttctcggcgatgcagaagaattgtgggagtgtaggcccgcccctgtaattagagcttatgaaaacacattgaagcttgaggcagaattttataagaatagggaagaccgctactgccgtatttatggaaatcacgatgattactggagggatacgggtagtattaatataggtggggaacaaataacggcaagggataagctgatgataagcctgcatgataaaaacaataaatctattggggacattttttttgttcatggtcatcaagggacttttgaaagcgaccgctttgggaagatcagcaggttttttgtaagaaacctctggagaccctttcagaggattactcatataaagttgactacacctgccaaggatgcagaattgcgcggcgaacacgatacggcaatgtataattgggctttaaataagaataatgggcaaaaaaagcttttgctgattgcaggccatacgcatttgccggtattcgcatcaaaagatcatataggaaagattagcgatgagcttaaatctcttcagcagcagcttgataatgcgaggacatccggcgatccccatgttgacggcattgcagaacagacagcggaaaaacgggctgagcttgaatatcgaaaggcgcaaaagaatatgaaagaggatcagaaaggcatttttatggagaagccatgctattttaattccggctgttgttcattcagcgatggagatataacaggattggagattgctgacggaaaaataaagcttgttaagtggcccaaagataatccgagagtagaagttcttgaggatgccgacttgagaagcgatatttttggaagcttatagcattgctgtaccttaagttttttaagatgacgtttgtgaaaggagacggcaatgatttacgaatacgagatagacggcttgacggtgcggacaggcgatatcatctgcacttctgacggcgctgcggataccgatatcaaaggccagttctggcggctgctggggaaaataatacccggcgaggtcgatcacattattatttatgtaggaccaaacggtcgttgcgtagaagcgggagcaaaaggcagggtgataacctttgagattatggatactacatgggattttcagaagatgattgcaaaaaggggaataatagataccctgtatggagcagcatatccgcttcaggggaagagtctgagcgaaacggagataacaggaatacgggaagcggttgccgcgtattgcctgaggcaggctgaattggagaagccgtataatatgaatttccttgattcaaccaccgaagatgcatactactgcagccagctcgcttaccttgcatatttcaaacacggcatcgacctgaataccggcaagggaatacccgaaataccagggactgagcgcataatcttcccgcaggagatatggagcgggtgcgggcataaaaaagcgcggtaaacaagtttcgctcttgccaaaatccatgcttattttgtagaataagagtctaaatttttattttcaggagggtttgcgttgaaagagggaatacatccgaaatataaagaggtaaaggtcgcctgcgcctgcggagagacttttactacaaagtccacaatggacaatataaagctcgatatctgctccaagtgccacccgttcttcaccggcaagcagaagatattggatgccgagggaagggtagaaaagttcaaaaagaaatacgctaagaagtagcgaataaacatatcggggttagagcgcggggtcgtttgcgctctaaccttttttttattaaggagatatatgcataagataggcggacaggccgtaatagaaggcgttatgatgaaatctcccgaaggctggagcgtggccgtaaggggcccgaagggcgacatcagctttaaaaccgtagtaacgaaaaaaccgaacaggtttttcaggctgccctttgtaagaggcgtggctgcccttattcaggcgctttccatcggcgttaaggctatcgaattttcgggaaatatcgcgtatcaggaagacgagaaacctataggcacgctcggcatgggactgaccatcggtttcgcgattattttagccatagcccttttcaagttccttccgctttttctcacgactttaatcggaggcgtttctgaaggcgtttcaaaaagctcctttatgttcaactttatcgacgggacgattagggtgggaatatttcttttctacatattcgccatcgggctctggaaagagatgaggaggatatatcagtatcacggtgcggagcataaggttatttacgcttatgaagccggggaggaactcacggtcgaaaacgcgaagaaatataagccgtatcatccgaggtgcggaaccagctttttgctgatagtgatggttatcagcatgctcgtattcatgtcgatcccgcagggctggtctttttccggaaagctgatgtcaaggattgtcctgttgcctgtgatagcgggcatttcttacgagacgctgaggctttcggcgagaatgaaggataatccggtcgtagggctgatcgtccttcccggacttttgctccagaggatgaccgtgcgggagccggacgacacgcaaatagaggtggcgctggcggcaatgaacgaagtcctgaaattaggcgcggataaagaggtttgcaaacagtgttaaaaaagctgcttacgatagaagaaaaatacgaagagataaccaattcgctcgtaaatccggatgtgttatcgaatccccaattgtatcagaaatactcgaaggagcagtcggatctttttcccattgtcgagaagataaaggagtataagaagcttcttgccgacctcgaaggtgcggaggaactgcttaaggccggagacggcgatttaagggatcttgctcaggaagagatggaggagcttaaaaagaggaagcccgttgtggaagacgaattgaaaataatgctcctccccaaagatccgagggatggaaagaacgtaatcctcgaaatacgggcgggaacagggggcgaagaggccgccttgttcggcgcggcccttttcaggatgtattcgaaatactccgaatctaaaagatggaaaatagatattatagattcaagcccgacgggactcggaggcttaaaggaagttacggctaatattacgggcaaaggcgcttacagcaggcttaaatacgaaagcggcgttcacagggtgcagcgggttcccacgaccgaggcgtccggaaggatacatacctcggcggcaactgtagccgtgctgcccgaggccgaagaagtggacattaaaatagaggagaaggatttgaggatagataccttctgctcttcgggcgcaggcgggcagagcgttaatacgacatattccgccgtaaggataactcatatccctacagggcttgtcgttcagtgtcaggatgagcgctcccagttgaaaaaccgcgagaaggccatgaaggtgttacgttcgaggcttctggaactcgaaatagaaaagaaggaaaaagagcgtgccgccgacaggaagacgcaggtcggaagcggggacaggagcgagaggataaggacttataattatccgcagaaccgtatatccgaccacaggatagggcttacgcttcataagctcgaacaggctcttgaaggaaatctcgatgagcttatcgacgccttgataacccattatcaggcggagaggcttaaggaattgtaaaaatcgtcaatacgggatcgaattctgctataatatttacaaaacagaaaggggcggtacgcacatagacagcaggactaaggcattagaggcggcgcaggcagcgctcgataaaaaggcaaaagagacggtaatcctcgaactgaaagaccttacgataatcgcggattatttcgttatctgttccggcgaaagcactacgcaggtcaaggcaatcgtcgaaaacatagagaagatattaagagagcacaagcaaaggccattagggatcgaggggctgaggtccgccggatgggtcttgatggattacggggatgtgatagtccacgtgttcgaagaagatacgagggcgtattacgagcttgagaaattctggctggacgccccgaaaatagcggtagaagacaagtccgctaagaaggccgcgggaagcaaaaagcagtatgtatgaaggcgcggatattatgggtcggaagaacgaaggaaaaatatttgacggaagggattaaccgttacctgaaaatgttaaagcatatggcgaatgtttccgtcatagaggtgaaggaagagaagggcaagacgaaggagaacgccctttttgcggagggagagcgtatattgcgccaaacagcgtcttatatcctgctcgatgagaccgggaaggaattcagttctaaggatttcgcgaagcttctcggcgaaagggatcaggtcgattttgtgatgggaggcgcgtacggggtttcctctgaagttaaggcaaaggcaacgggtaaaatagcgctctcgaaaatgacctttacccatgagatggcaaggctgatatttttggagcagttttaccgcgcgatgacaatcgtcaaaggcaaggagtatcatcactgatgggcaagatagcgatacttgtatttttgttgttcctcggagtcctcggtttttttgcggtcgagaacaaagacgccgtgatcatgaaaattcccttcggcgacgcttatgagatcccgaagatagcgcttgttctcctttcctccaccataggcgcgcttgccgtccttatcgtgttttttataagagatacgaaacgcgtaatcgaaaatctccagtatcagaggaggcagaagaaagaggccaggattcaggaattttattccaaggcgttgaacgccataatgggcgataaggaggaggaggcgcgggaagccctcggcgatatattgaaggaggacccggaatatatagacgcccttttacggctcggcgacatctcgctcaataacggaaaatatgaaaccgcgttcgattattacaaaaaggtgaaggacataaacccgaagaatctacaggtgcttttttccatggaaacagtgatggaaaagctgcagagatacaacgacgcgtttaattacctcgaagagatactcgatctcgatcccgaaaatcttaccgggctctacaagaagcgctctctcctcgaaaggaaagagatgtgggacgacctcctttccacgcagaaggcgataatcaagctcgaacataacgagcacgacaggcagcgggaagagcgcaggatgctcggttataaatatgaatacgcccgcgcgagccttgaaaacggagatgttgaaaaggcggaaaaggcgttcaggacaatgctcaagatggacggtaattttgtcccggcgcacctcggtcttgcggaagtcatacttatgaaaggagagaccgaggaggcgataaactttctcgaaaaggcgtacgagcagctcaggtcgataataataatcgctcgccttgaagaccttctcatcaacgtaggagaaccgggcaggcttataagattctataaaaacgccatagctaaaaatcctcaggacaacgggctgagatttctcctcggcaagctctactacaggcttgaaatggtcgatgacgccatggagatgctgaattcaatcgataccacggtcttttcggtgcccgagctgtatagtttgaagggagagttatatataaagaggagccagatacagaaggcgctggacgaacttaaaaaggcatgcggcataaaaaggccgtttaagatccaatactgctgctcaaactgcggt

SEQ ID NO: 55 - DNA, 1984 bp, Nitrospirae bacterium GWA2_46_11
ggatgatgttgcagacatccttaaaggacgcagggaacagagaggtatcagaagaactcagcagacaaggaagaaaagactgcgggagcttaaaaaatatctaaatagtataggatatgataattctaccgaactatttaaatctatttatagcctcgcccataagaggggctatgactatgccgatatgccaacaccggaagaaatagaggaaatggatgagaaggaaaaaaaagaatggaaagatacgcagagaaattcaaggtacaggaaagaggttttggcagatgtccgcaaggtaatgtccaatggtggagcttcggatgaacagataaagagggtagaaagtattttcaataagcagtatcggcctaagagatttaataacaggattttgaccaagtgcaaggtctgcggaaaaaatacgccattaagaagaaatgtgagggagttgcttcttgaaaatatagttcgcttcctgccgttagaaagcgaactaaaagaaactcttaagcgaacaattcttgaaggacagcaaggaaatatcaataaactattcaggaaattgaaatttaaccagaaagactggccgggcaaaaatcttacggacatagcaaagaacaaactgccgggcagactgcctttttgcaaggaacattttgcagagaatgagaagtttaccactatagagaaatcaactttcaggcttgcgccttctttaaagacaaagatagaaaatgtcctgactgtgattaaagacgaagtaatgcctaactttgctctcgatagggtagtaatggaatctaacaatttcgatatagctgcaaaaacgaagggcaaaaagaggttggcaaaggaggaatacagcaaggggcataaggaaaatagagaaactcttatggaatcccttttaagggagactgatggaagatgtgtttattgcggtaagactataactttagccgatgcaaataaagaccatatatatcctaagaaagcaggaggcagcaatatctttgctaaccttgtggcttgctgcaggagttgcaatgaaaataaaggggggcggactccatcggaaagcggcattatgcctaatccagaggtcgttgcaacgattaaaaacgatttaaagaaaaaaatcctcgatgatgcaagaagcataaagcagttagatttcaacaaatacatgtctcatgcctcgattggctggaggcatatgagggacaggctgaaagagctaaccggaaacgataaattgcctgtagagcggctgagcggtattgttacagcatactttagaaggtggtggggatttaagaaggaaagggctaacgataagcatcacgcgcttgatgctgttattcttgcttcaaggaaagattatacggatgaggggctggtggctatgacgcttaagcctgcaaattcggatggacgggagttcgaccctgaaaagcatatcaaagaatcggaagagtttaagagaaacaagggcagcagagggtctgccctctacgacaaaaacccgttgtccattaagaatggcaaaattgctcgaagatatatggttacggagattgaaagagggaaggaagatgctgttatttctgaggaatggcgtgaaaagctgaaagaagcatttgaccgcttcggggtcagcaatggaaaatgtttaactgatttacagactaaggaagttgggttatatggtcagaaaaatcctatgagccttaaatgcgctgtcaggggcgcaggcaaagggcagattgtgctgataggcaacaatgcatttaagacgaatgttcataatgtcggcgtggcggtttatctcgacgaaaagggcaagaagcgggcatgcgaacttaaaaaccagagactggcaaaacactttgtagagccacaggatgagattaaaggaaagatattatttaccttgagaaaaggggacacagttaaggcggaagatggaaatatttacagg

SEQ ID NO: 56 - DNA, 1015 bp, Nitrospirae bacterium GWA2_46_11
aaacgaagcggagagcattcccttattgatcgacaaagatgttgacgacgagatatttatcgatgaagggatttctctgataaaaagcggcgattcatcgcagatagtgttatcggggttcggcatttttttaagcaaaaagagcgagcgtcttattgtaaagaaaaacactaaagttgtttatgagtttccctttttcaggctgaacgaagtcatcatggcttcccgcggcatatcgctgtcgtcggatcttatagaggaattgtgcgagaggggtataagactgaattttctgaacaacaacggcaagccgtacgcaatgctttcttctccgatgctctccgcaaccgtcatatcaagaagagagcagattattgcctttaccgatacgcgcggtcttcaattctcaaaagctattgtggagggcaaaatcagaaatcaagaaagacttttgagatatttcggcaaatatattaaaactgttgacagcgccaggtttgacagaatagaggagataacagatgtcattaggaagatgagcaggcaggtggaaggtattcacgggcagagcattgaagatgcaagaggcagactgatggcaattgaaggcgcttcaggtaggctatactgggacggcgttaaggagataatcggaaatagaatagagtttttcgggcgggaaacgcggggagccattgatgccgtaaattccctgctgaattacggctatgggattttatattcccacgtctggggcgccgttataaacgccgggcttgagccgtttgcggggtttcttcatgtggacaggcccggaaagccgtctcttgttttggatttggtcgaagagtttcgtcaacctattatcgataggattgtaatcgcccatattaatcttggagaatcaatcgaaattagaaatggcctgcttgaagctgagaccagaaagctgatagggaacaaaattattgaaaggctggagagtcaggaaacacttgacggcaaaaaatataaa

SEQ ID NO: 57 - DNA, 7096 bp, Nitrospirae bacterium GWA2_46_11
gtaatgaggattcctgtaaaatgccgtgtccgtgatcatgaacgccgaatagccgaactcccagaagccccagtggtccgagaaattaacgccggggatcagcgaaatcccgttgaacgattcagcgggcagtgaagatgactcttgaaaatatctcttcacttttttcgtaaaagacatggagcggatatttccgacaaacgtaataaaattgcccttgtcaggataaaaccatttgaatatcggaaaaggataatactgagagttttttctgtcggagtaatatccgagcatttcgagggagatcatgccgcagacttttatgccttcctgtttaaggcttttagcataaacgtagcttcccatatgttttgtcataaaagtgggaggctcttcaagactgaaggcaacaaagcggaccgtcctttggaaagattttgttacggcaagccgcgccagttcgagcagtcccgcaatgccgcttgcattatcgtccgcgcccggagtgcccgttacggtgtcgtaatgcgcgcctattacgagaatcccgtcttgcgggtttccggcgccttttatttcgcaggatacgttgtaatatgtattgccgagatacccaaacggctgctttctaacctcacagccgtaagagcgaaattttttttctatgtaatcggcggccttgttgagcttatctgtatcgcgataactcctctcgcctatatcgtgggcgaggcgcttgacggtttcgataagattagcttttatattttccattttgccgattaatgttcatagctttataaattctttcggcgctaaattcgtcctcattgtccataccgatgcagtgccgtttttcaagcgatacacaaccagcgggtcatagccgtattgctccacaaagggtttcagaaattcgcgcttttgcactatctccttctttagttcaatatcttccgtaagttcaacgaccccgctgactcttatttgtataaggttctcaaagctgccgttgttaaagctcagctcgaccagcgggttttcggtcaattgtttatgcaagtctttcacctttcccgtgtgaaacaaaataccgttctcatcagccctatacatcagcataccgcgaacgcggggcttatcaccttcaatcgttgccatgtgaaataccgggtttgcattaagaaactccaaaatttcagtcttgttcatataagcttcctcctaacgataaatgttttcaagcctgcggataatatcgagtttttctttctctgttgttctcactcagggttactccatttgcggccctttgcctatcttcattttgcccctgaaatatctttgtttgacttccgcggtcttttccttccatttgtccacgccgtgctgtttaatgtagcacaaagcaaatagtttaatgttgtgcggcaggtcgccggccttgtccgttgcgggatttaatttggcgcaggggaattccgcgcactcgaagcagaagtcatgtccgcgtttgctgatgcaggcataggtctcgcatgtgccctcaacaaactggcagcgtcccttgagcggcctgcatccctgacatgggatgccgttccagttaaattttttggtcagcatttctctccacgaaggatcgtcctttactttgtacgctgggcagtcaccgcagtatcccccgcacggcgcaactaatgtgatgtcatcttttgtttccattcgataagcctcctgtagccggtctgtatacctttgcctggttgtcgcgatatagttgatcgaaaagttcagtaaattgggattgtccggccatcatatcctcttcttagattacacagcactaagctgttttttcaattcaggagtgtgagttaacatcgggacagcgaccaggaaaggccgctgtcccgttgcggtcatactaacaacagcccttgtctgattttgtcccgcagcaggactggaacatgttcttcatatttccatccgtgcagcacttctcaaaaatggttttaattttttcttttacgtcttgaccggtgatctcaagacgatacccattctcgagttccgtaacgttagcgctacagcattgtttctccattattattcacctccttccagtcttttattattcgagaacactcgaacaagaccgccaaaaaaaactattccatgaacttttttatggaatcgatcacgtccttatttactctgcaacgatagatctgcccctcgcgctcacatgttataagcccggcattttggagttccttgaaatgatgcgatattgttgaccgtgaaagattcatgcaatcacagatctttgtaaaggccttttccatgcttccatcagacgcgcaacatgttgcccctttaatgcgaaattccgccccggccttggacccctcgattccgcaacagtttttgtagagcataatgaatatcttcaatcgctgctcatttgaaagggctttaaatatttttgtgtagttagatagtttcacaacattagaataaacttatcgtgcgtttttgtcaagcattttgacgagaggtaaaagctttggcgttattatcaagagtatgaaggagtagggggataaaatcggctgaaatttttacgaatgatataccgaagccttatgaaattcttcaatcgccatatccttatcgtatgagaagccggcaggaaaaatgcccttctttttgcaatacacaaccatctcatccagcacagccatgagttcatctttcaggttgttcacttcccctaagccgtcacaacccctctgcctttcttttataaaactatatatctgaacgtagtccttagcttgtgcgtagagtcctgaaagatgagaatcggattccgccattttagtaattgcatcaatcatgcatacacctcctttcgactgtatagacgaaggaaagtgggaaaaggatacacccttattttttcttgtggaattttaaggtgggttctttgcggtcgcaggcgaattcgttccgctcgtcgcggtagaaactgcagtgcgcttccagcgtttttttcagccttgcccgcgcacggtggagcctgatcttgacggtatcaaggcttaccccgagaatctctgctatttctccgtttttgaacccttcaagctcgctcaggacaagtatggtccgggaatgagggggaagggcatctacatacccccggatacattcgctcatttccttgcggatcaattgcacatcggggggaggagtttttttatcataaaaaacatctgtttcttcattatccataccgagcggctgttcttgtctgaacgacggactgcgcatcctgtcgactgcagcgttcgttgcaattcggtatacccatgtcgacagccgggaattttttctgaaatccctgagtccacgatgaacctttacgaaaacttcctgggctgcatcctcggcctcgccggccccgatcaaccgctgcaggtaacggagaacaggcgtcctgaattcattataaatttgttcaaatggcgtatcgttggtcatcgattgttgatctgctttttaatctcctgcataggagtcattccttccccaaaattaaatcaaccatgattttacgaggttggcagaaaagtttttttactctttccctatattcttcgggtgtaacgtgttcctgatgagttcctatcacgattttaatattcgggaatgcctcttccaaagccgatttgtatgtctctttaaacgggcatagcgccttcatgcagtaagtaaagtgtatcgcatccacccggaattcagtcagtgcccttatacgttgcaataatttatcggcgccgctaagggtagggcagcccggacaattaataatccctaccaaatctaacgggtcatcttttggatagtccgcaaatgttcctcttctttttctgagatcggcaagacagcttgccgacgaacagcccaagtcctgcgtcgcattgctgcatgtcaatattccgattcgcgccatataatcaccttctcgtttacagtttctctaaaaactctttcctcacaggcgaccacgcgtcaacggccttgcaaggcatgttgcctgtaaaggccgaatgaacggcgtttgacggtatcgcgattacatcccctgccttcaaggtagttgtttctccttcataggtaaaggtcaattccccttccagtatcagggtgatctgttccgcctcgtgcgaatgctcaggaaattttgtgtccggctccatctcaaaatatgtaagcatcgccttatcgagagctattgcccacatcgaggctcccgggacattgcttctgagctttaattctcctttgcgataaatatgtatctcgcccattgcttctcccctttttagcgatttcatttttttagatatttcatctgaatattccagagtataagccccgcgccttcgggtctcgaaggcagaaattttctaatttcagaggcgataaattcggcattgtcggatttggccgcaagctctttggcctttttatcaaaggcaataagatagtctttcatgtcttctatatctttttttgttgagaccggcccgtgccccggaattattttctcgacgtccatcgtcatgatatagtcgagcgtttttaaccatccctctatatcgccttctcccatgaacgggtgataaccggtaaatagaatatcccccgcgaatagaatttttttgtccggcagatagaccataatgcttccatcggtatgcgactgcctgaaatagatgagttctattttccggtcgccgaggtctatttccattctttcgttaaacgtcaaaacagggtatgcgatcttcgtccctttcacatccttttcggtaagtccgtaggtatttgagtttttcagggctgtttcgccgcgaattttcagattattaccgtcattagtgtgcgagatgatcagcgctccgagttttgcgaattccgagttgccgaacgtgtggtcgaggtgataatgggtgttgaggacatatttaacgggtttcttcgagactgaccttatgtccctgataaaccttttcgcctcttttgcggatatgagggtatctactaccacaatgccgtctttaccgataatgatgcccgcattagcgccgtagctgttctgcggagtgctgtttttagtatccacatacgaataaacgtcgtcggctatctttgtcagtttctccgcggcaattgcaatttgaccaaatgctaaagaaacgatgaatacaaatacccaaatcctgaaaatcatcaaccttttcataacatcctcctcgttttttattttaatcctttaccgaatccttcaacagtttcctgaaaacaaccatattcgattttgtgaagccgtgcttccgataaaattcatgcgcctgctcgttatcattatcggtaagcagggtgatccttccgcatccgttattaatcgcaaattctaccgcatgcttaattaaacttgtacccacgcctttcccccttgaatcgggcgatacaaccatatcttcaagaagggcgactttcttgccgagggcggtacttatcgtgaataatacgactaccattccctgaatgacgccgttagattcgcatacaaatacagagccggtttcagggttcttaacgatcatctccaatcccctcctttgagccgtcggattgggtttaaactcatgctcttgaccgaaaagaaggccgagaagctcaatgcaagagccgagatcgtctgtatttgcaggtctaacggttttcattaatatgctttctctcgtccgatcacttcattctcaagaattcttcaatcaccggacacaattcgctccctttttcttcctgcacaaaatgtcctacatcatcatattttattgttttggaatcggagaaaaggcttatccatctgctcagttcctgctctctgaaagcaatgtctttcattccccataaaatgagagccggcttatcttttattttatcactctgcgaccagagaaaatccagccaatcactggaatcaataatccgcttcggaaatgtccagcaacccttccgttgctccggtatttcaagcgccttgaaataatgctgatggattgaagtggggagtttggaggtatcacccatcgcctttttcattatgacccgcacaaaaaaattaaagcgtttgatcaagaacctgccgattggtccgcccacaaatttgctgaacctctcataataaggatctcccttgacagaccacatccaggtattcatgatgatgatacgtttcatattttccggagagttgacggcatatgacaagccgataggtccgccccagtcctgaacaacgagcgttattcctttcaaatctaatttttctatcaatagtttcagatttttcgcatgatcttccggataatacgaccaattccacggcttatccgataaaccgaacccgataaggtcaacagctatgcagcgataatttttcgagagacatttaatcagatgcctgtatagaaaagaccatgcgggattgccgtgaaccataactatcggctctccgctgccctcatcaacataatgcattttccccatttcaagttcaagaaaatgagacttaaacggatactcttttctgtctatccacttattcatgaaacctccttatctctcatctcctttttcgctccgtgttcatagcctgaccgattcgcgccgttataaaccggtatttgccaagtcggtccatggagagagaactaccgccaaacatttcgattatatttccacaacccattcttttatccagttgctctcaagtaaatcctgaataacaaaagggtcgtttattataatcctatttgcatcgtcaacgctttcggcctcaaatgtaatcattccacccgtcctgtctgcaaaaggccctccgagatacttgctcaatttaagcttgtgccaatactcaatatgcaacggtacagcatcctttatcttttccggctcctttttcatgaaataaaaaaatgcaaattgttttcccatgccatcctccctgcag

SEQ ID NO: 58 - DNA, 4915 bp, Nitrospirae bacterium GWA2_46_11
ttaaaatccgaagactggtcgggaaggtgtcccggatgcacggagtggaatacctacaggctcgacgtttatggctcctgcaaagcgtaaagacagcgatgagttatcagtgatgagtgatgagttaaaaaacaaaaaaaaatcaaagacgaaaagtaaaagctcaaaagcaagcacatcaccgtcgttaaaagtcgtactcgatacgagccttttcgtaaatccggacgtcaggacaagcctcggagcaacccccacagaggctttggaaacattcctttttctggcggcgcagatacatatactcgaattctacatgcctccgtcgatattcgaagaactcctgcattttgtggagagggacaagataccggccgatctccttcttatcttgcatcagaaaccgcctaaaaaacatgaactcacgactcccgcctttctcttatacgaattgattgaggacataagggaaagggtgaacaaggggctgcggatagcagaaaaggccgtaaggagtacagatcaaaaaaagacggatgaaatcgttcaggatatgagaagaaagtacagggacgcgcttagagaagggatcatcgacagcaaggaagacgtcgatctcctgctgctcgccatggaacttgacgccctgcttatcacggccgatcaggggctgataaagtgggccgaaaaattaggtgtaagatggctgttcccggagaaattcaaggaatatttaatgagcgcgataaaaagaaccgaacttctttccgcaaaagaataaagtgtagaaacaatgtagttttgggaagattctttgatggaaaaagaacttgttttaggcatcgattatgggggcaaatacacaggccttggggtggttgaccgcaggaacaatcaggtgttgtatgcgaggaccataaaaatgcgtgatgatgttgccgatattctgaaaggtcgtcgtgaacagcgtggtattcgtcgtacccagcagacccgtaaaaaacgtctgcgtgaactgaaaaagtatctgaacagcattggctatgataacagcaccgaactgttcaaaagcatttatagcctggcacataaacgcggttatgattatgcagatatgccgacaccggaagaaattgaagaaatggacgagaaagaaaaaaaagaatggaaagatacccagcgcaatagccgttatcgtaaagaagttctggcagacgttcgtaaagttatgagcaatggtggtgcaagtgatgagcagattaaacgtgttgaaagcatcttcaacaaacagtatcgtccgaaacgttttaacaatcgcattctgaccaaatgcaaagtgtgcggtaaaaatacaccgctgcgtcgtaatgttcgtgaactgctgctggaaaatattgttcgttttctgccgctggaaagcgaactgaaagaaaccctgaaacgtaccattctggaaggtcagcagggtaatatcaataaactgtttcgcaagctgaaattcaaccagaaagattggcctggtaaaaacctgaccgatattgccaaaaacaaactgcctggtcgtctgccgttttgcaaagaacattttgcagagaacgaaaaattcaccaccatcgaaaaaagcacctttcgtctggcaccgagcctgaaaaccaaaattgaaaatgtgctgaccgtgatcaaagatgaagtgatgccgaattttgcactggatcgtgttgttatggaaagcaacaattttgacattgcagccaaaaccaaaggcaaaaaacgcctggcaaaagaagaatatagcaagggccataaagaaaatcgtgaaacactgatggaaagcctgctgcgtgaaaccgatggtcgttgtgtttattgtggtaaaaccattacactggccgatgccaacaaagatcatatctatcctaaaaaagccggtggcagcaacatttttgcaaatctggttgcatgttgtcgcagctgcaatgaaaataaaggtggtcgtaccccgagcgaaagcggtattatgccgaatcctgaagttgttgcaaccatcaaaaacgatctgaaaaagaagatcctggacgacgcacgtagcattaaacagctggatttcaacaaatatatgagccatgcaagcattggttggcgtcatatgcgtgatcgtctgaaagaactgaccggtaatgataaactgccggttgaacgtctgagcggtattgttaccgcctattttcgtcgttggtggggtttcaaaaaagaacgtgcaaacgataaacatcatgccctggatgcagttattctggcaagccgtaaagattataccgatgaaggtctggtggcaatgacactgaaaccggcaaatagtgatggtcgcgaatttgatccggaaaaacatatcaaagaaagcgaagagttcaaacgcaataaaggtagccgtggtagcgcactgtatgataaaaatccgctgagcattaaaaacggcaaaattgcacgtcgttatatggtgaccgaaattgaacgtggtaaagaagatgccgttattagcgaagaatggcgtgaaaagctgaaagaagcatttgatcgttttggtgtgagtaatggtaaatgcctgacagatctgcagaccaaagaagttggtctgtacggtcagaaaaatccgatgtcactgaaatgtgcagttcgtggtgcaggtaaaggtcagattgttctgattggtaacaacgcctttaaaaccaatgtgcataatgttggtgttgccgtgtatctggatgaaaaaggtaaaaagcgtgcatgcgagctgaaaaatcagcgtctggcaaaacattttgttgaaccgcaggatgagattaaaggcaagattctgtttaccctgcgtaaaggtgataccgttaaagcagaagatggtaacatttaccgcatacttgaacttggcgaaaggcctgttgtagacataaagtgggtaccaacatctgatggcaagaaaaagagagtaaagacagctatacacgcaacaaagctcacaaagctctaactgaaaatttttttaatgtcctaacccacaatgaaggtgtcattatcgactgtagttttagtttggcttaggctggacaaacccaagcgtacattccttcatcatataggcgttttacaattaaggctctttttgattttcgcaaaccccctggggtttatttcaaaagatggcttttagaaaattattgaaaattttctctgaaaacttcttgaaaatactaagctataaggacggtttcaatcatcattttcaaagagccttaactgtaatgagaagtcatttaataaggccactgttaaaaagcgtttcaatcatcattttcaaagagccttaactgtaacgctcatataagcaaggtctgtccgccggagaggacggtgttttctccatcggtcgcgctactgaaccaactgactgtcagttagacgaaacaatcagaacagagtccctgccggaaaccgtttccggttgtccagcgaacattgagaaagctgcgacgccagatgcaccttccggtgggaagtcctccgctcaaacgcctgaagcgtttcgcagcctttctgaaacaaatcaaaaaagctgagacaagattcctttaaaatagagcatctcaaagaaaagtccctgcgagcaatgcgcttcttgaaggaagcccctctctaacgtccgaaacgctcagggacactaaaatatatctcagaattgtaaaagagcctaaactataatatgattacattatatgcctttaaatttcaagagctttagcggaggtggatatgcaaagcaatacttttaaaatttcacgcatccatcctgaatacgtgcctaaagaaaaattgcctcttgcccaattcaatctttttacaattaggtcgataatccagatgcaggccagaaatttagcggcctttctgaacggcaaaagagaatacaaaacgttcagatttaagtggtaattgatggaagaacatacagtctatattttttacgatatagaagatgacggcatacgcaataagattgcggagaagtgtaaagactatgggctcgaaagaatacagttcagcggcttttccggcattttaaatagaaacaagcgggaagaactatttctaaggctcatttctttaattggcggcaaatcaggcaaaattcttatgcttcccttatgtgaaagggatacgaaggtaagaagggagtttatacaggaaggacaagatgattcaactgagggtcggtgatctcaagcagttcgtatattgtccaagagtcgtgttctacaattatgtgatgcctgtcgagaaagttccaaccttcaagatggagcatgggaagatagcagaggataaaatagacaggcttgagcacagaagaaaacttaaaaagtacggcttgtccggcggggaaagggaattccacttgaatttatattctccgaaatatgagttatcaggcaagattgatctgcttatcaatactgtcggttcttattatccggttgactttaaatataccacctcgcagccgcataaaaatcatatctatcaacttttggggtatgccctaatacttgaagacatatataaatgcaaagtcaataggggatttgtatatataattcccaaagaggatgcaatttcttttgacttaacggatgatttgaagggcaaagcagaaattttgcttgatgagatcagagatatgatagcattgcagcaaatgcctgcccctgcaaactccagaaataaatgtgacgactgcgaatataggaatttctgcggagatattttttgacaattataaaccgccgagggctttatacagccctatcacgaattccgtcatcctctcgtaatcgagcgtttcgaatgtatctcccggcgcatg

SEQ ID NO: 59 - DNA, 35 bp, Gammaproteobacterium AqS3
gttggatcaggacgtcacttttcgaattcgaaaac

SEQ ID NO: 60 - protein, 807 aa, Gammaproteobacterium AqS3
MKQLTLDVGIASIGWAIVSKKGNVKAGSRIFPDAKSGRESNQSRRAARLMRRGYRRKAKRRADTLAIIRSIHPGFDPEGHPDIEREALIKAIITPGAPTPSLDQLACAFQRFAKSRWPQYSRTLPKRTEREDQFIQVWVIAERTYPDRFTPDVATRLLQAIFFQRPIKDGDRAKCQLFRHHGDKAPLVGWTHEPELQRFAILSDLSNLTIGIGSTDNLLCEYPDIIEDLETRCFETGMSWREIAEHVKEVIGKGVVFRGIDGQKKVGRNGIGPAKLETIDEEGNSTKSTASMSVEAAVMIYHQMKADRCRAATAKKTLIDAGALSAPLTAKDIKRGDRTLTITELMDMAGRITDPTIRAIYHQVEMLVNELIARFGKPERIVIEAQKEIGRSIEDIEKAMAREREKHIERQRENRARNAAMGTKARFARLCAIRGDRCFISDRPAAEVGHLIADSIGGTLEMANLIPIDPAINKEMGNRTPYEAFRKTEYWSIIQRKLQALEDEVKALKPPKGTKGTAWTIYHRAKHQFDFFAWRFQSNARETHQRNFRPGSLDDLRWIENLLFLGVAPICDNIRIVSGRTTERIRREILGMDKDRRDHRHHALDALAIMLANPLKPWDLKSSNSLGIPLGRIKQAFADAVVSQKQDHSLRTALHKENAIPKTKRGAAYRKIGTGASERVVDTQSKAYCEVWALPNGKWEAVVVSSFDAAQKNYRQGIDHRPHPAARLVMRLFKSDLLGIGGKIYRVQELLGSGSIYLVDHRFAGTIRDARAVCKTGVNVDFFSKGGDSLRKAGARLVSIRKSWVGS

SEQ ID NO: 61 - DNA, 37 bp, Deltaproteobacteria bacterium GWF2_42_12
gtttcacgccatattttcaaagagccttaactgtaac

SEQ ID NO: 62 - protein, 747 aa, Deltaproteobacteria bacterium GWF2_42_12
MERELVLGIDYGGKYTGLAVVDRRHNQVLYANRLKMRDDVAGILKDRRKQRGIRRTAQTKKKRLRELKNYLKSIGYNESTATFETVYSLAHKRGYDYADMPEEKTSEEIEAMDVEERKQWEKEKQEWEETKRNSRHRKEVVKDVHKAMIEGRATEEQIKRVERIFNKQYRPKRFNNRILTKCKVEDCGVNTPLRKNVRDLLIENIVRFFPIEQSEKDNLKDAVLDKNRREEVKSFFRKHKTDEHIRKQVYDIADNKLSGRTVFCKEHILEHTEHSKEERKVFRLAPSLKTKIENVLAVIKDEILPKFTVNKVVMESNNFDIAAKTQGKKRLAKEEYGKGPREGKETRKEALLRETDGRCIYCGKSIDISNAHDDHIFPRKAGGLNIFANLVACCAVCNENKKGRTPLESGISPKPEIIAFMKNDLKKKILEDARNINTVDFNKYMSHASIGWRYMRDRLRESAGNKKLPIERQSGIYTAYFRRWWGFKKERGNTLHHALDAVILASRKGYSDDGLVDMTLKPKYNKGGGFDSEKHLPEPIEFKRDKGSKGSALHDRNPLSYKKGIITRRFMVTEIECGKEDDVISETYREKLKEAFKRFDTKKGKCLTDKEAKEAGFCIKKNELVMSLKCSIKGTGPGQMIRINNNVFKTNVHNVGVDVYLDEKGKKKAYERKNPRLSKHFIEPPPQPNGRVSFTLKRRDMVTVEGEDAIYRIKKLGTSPTIEAVVGSDGKTRTVSATKLTKANSAE

SEQ ID NO: 63 - DNA, 37 bp, Unknown; identical to SEQ ID NO: 61
  Soil microbial community from Rifle, Colorado

SEQ ID NO: 64 - protein, 778 aa, Unknown
  Soil microbial community from Rifle, Colorado
MEREIVLGIDYGGKYTGLAVVDRRHNQVLYANRVKMRDDVADILKGRREQRGIRRTAQTKKKRLRELRNYLKSIGYNESTATFKTVYSLAHKRGYDYADMPEEKTSEKIEAMDEEERKQWEKEKQEWEETKRNSRHREEVLKDVRKAMTEGRAAEEQIKRVESIFNKQYRPKRFNNRILTKCKVCGKNTPLRRKVRELLLENIVRFLPLESEMKETLKQTILEGQQENINKIFRKLKFNQKDWPGKNLTDIAKNKLPGRLPFCKEHFSENEKFTMIEKSTFRLAPSLKTKIENVLAVIKDEILPKFTINKVVMESNNFDIAAKTQGKKRLAKEEYGKGPREGKETRKEALLRETNGRCIYCGKSIDISNAHDDHIFPRKSGGLNIFANLVACCAVCNENKKGRTPLESGILPKPDVIRFMEDEFNRLKIKMWKIRRKQSLTTEEIMDKAKRNKLELKLKILEDAQQVNELDFNKYMSHASIGWRYMRDRLRESAGNKKLPIERQSGIYTAYFRRWWGFKKERGNTLHHALDAVILASRKGYSDDGLVDMTLKPKYNKGGEFDPEKHIPEPIEFKRDKGSKGSALHDRNPLSYKKGIITRRFMVTEIECGKEDDVISETYREKLKEAFKLFDTKKGKCLTDKEAKEAGFCIKKHELVMSLKCSIKGTGPGQMIRINNNVFKTNVHNVGVDVYLDEKGKKKAYERKNPRLSKHFIEPPPQPNGRVSFTLKRRDMVTVEGEDAIYRIKKLGTSPTIEAVVGSDGKTRTVSATKLTKANNAE

SEQ ID NO: 65 - DNA, 37 bp, Nitrospirae bacterium RBG_13_39_12
gttacagtcgttgttttcaaagagccttaactgtaac

SEQ ID NO: 66 - protein, 762 aa, Nitrospirae bacterium RBG_13_39_12
MEKELVLGIDYGGKYTGLAVVNQKNNQVLYARTVKMRDDVTDILAGRREQRSLRRTLQTKKKRLRELKNYLESIGGIYEESSGTFTIEPFRTVYSLAHKRGYDYADLPEEKTSEEIEAMDAKERKQWEKEKKELEETQRNSRHRDEVLRDVRNVMTEGNLSEEQIIKVESIFNKQYRHKRFNNRILTKCKVCGKNTPLRINVRELLLENIVRYLPLQNKERELLKLTILKGHQQDINEIFKHFRKVYKITLNQKDWPGKNLIDIARNQLRGRLLFCKVHFPENEKYVSIEKKTFRLAPSLKTKIENVLSVIKDDILPNFTLNNVVMESNNFDIAAKTKGKKRLLKEEYSKGHRESGETRKEALLRETDSRCIYCGKGIDLSNAHEDHIFPRKAGGINIFGNLVACCSVCNEEKRGRTPLESGILPKPEIVSFITNDLKKKILEDAQYINTLDFNKYMSHASIGWRHMRDRLRELTGNKELLIKRQSGIYTAYFRKWWGFIKERGNHGHHALDAVILASKKSYAEDGKVDMTIKPCGEDGKEFDIERHLSEMKEFRRDKGGKSAPLHDRNPLSFKNDIITRRFMVTEIECGKEAVIISEEYRKKLTEAFKRFGIAKGKYLTDEQAKDAGFYLRKNGEGVMSLKCEVKGTGYNQMIRIKNNIFKTNVHNVGVAVFLDEKGKKRACELKNPRLSKHFVKPAEQVKGKVIFILKRGNMVTVEGEEMIYRVKKLGTSPVIEAIVGSDGKTRTVSATKLLKINHTKKV

SEQ ID NO: 67 - DNA, 36 bp, Nitrospiraceae bacterium isolate UBA9935
gtttcaatcatcattttcaaagagccttaactgtaa

SEQ ID NO: 68 - protein, 735 aa, Nitrospiraceae bacterium isolate UBA9935
MEKELVLGIDYGGKYTGLGVVDRRNNQVLYARTIKMRDDVADILKGRREQRGIRRTQQTRKKRLRELKKYLNSIGYDNSTELFKSIYSLAHKRGYDYADMPTPEEIEEMDEKEKKEWKDTQRNSRYRKEVLADVRKVMSNGGASDEQIKRVESIFNKQYRPKRFNNRILTKCKVCGKNTPLRRNVRELLLENIVRFLPLESELKETLKRTILEGQQGNINKLFRKLKFNQKDWPGKNLTDIAKNKLPGRLPFCKEHFAENEKFTTIEKSTFRLAPSLKTKIENVLTVIKDEVMPNFALDRVVMESNNFDIAAKTKGKKRLAKEEYSKGHKENRETLMESLLRETDGRCVYCGKTITLADANKDHIYPKKAGGSNIFANLVACCRSCNENKGGRTPSESGIMPNPEVVATIKNDLKKKILDDARSIKQLDFNKYMSHASIGWRHMRDRLKELTGNDKLPVERLSGIVTAYFRRWWGFKKERANDKHHALDAVILASRKDYTDEGLVAMTLKPANSDGREFDPEKHIKESEEFKRNKGSRGSALYDKNPLSIKNGKIARRYMVTEIERGKEDAVISEEWREKLKEAFDRFGVSNGKCLTDLQTKEVGLYGQKNPMSLKCAVRGAGKGQIVLIGNNAFKTNVHNVGVAVYLDEKGKKRACELKNQRLAKHFVEPQDEIKGKILFTLRKGDTVKAEDGNIYRILELGERPVVDIKWVPTSDGKKKRVKTAIHATKLTKL

SEQ ID NO: 69 - RNA, 41 nt, Artificial Sequence
  Synthetic
  misc_feature (1)..(20): n is a, c, g, or u
nnnnnnnnnnnnnnnnnnnnguuggaucaggacgucauucu

SEQ ID NO: 70 - RNA, 181 nt, Artificial Sequence
  Synthetic
uugcaaaaaaugauguccugauccaauaaggggcgcagcccccccaccauuuuuccccgaucuacggaucggaucggcgcugccuucgaaaagccgacauggaaaacccccaguuuaucggggacgauguaaaccugagucgccacggcgcuccucgcccgcucuacggaccgggcuucgs

SEQ ID NO: 71 - RNA, 226 nt, Artificial Sequence
  Synthetic
  misc_feature (1)..(20): n is a, c, g, or u
nnnnnnnnnnnnnnnnnnnnguuggaucaggacgucauucuaaaauugcaaaaaaugauguccugauccaauaaggggcgcagcccccgaccauuuuuccccgaucuacggaucggaucggcgcugccuucgaaaagccgacauggaaaacccccaguuuaucggggacgauguaaaccugagucgccacggcgcucaucucgcccgcucuacggaccgggcuucg

(19) United States
(12) Patent Application Publication
(10) Pub. No.: US 2022/0403357 A1
(43) Pub. Date: Dec. 22, 2022

(54) SMALL TYPE II CAS PROTEINS AND METHODS OF USE THEREOF

(71) Applicants: THE BROAD INSTITUTE, INC., Cambridge, MA (US); MASSACHUSETTS INSTITUTE OF TECHNOLOGY, Cambridge, MA (US)

(72) Inventors: Feng Zhang, Cambridge, MA (US); Soumya Kannan, Cambridge, MA (US); Han Altae-Tran, Cambridge, MA (US)

(73) Assignees: THE BROAD INSTITUTE, INC., Cambridge, MA (US); MASSACHUSETTS INSTITUTE OF TECHNOLOGY, Cambridge, MA (US)

(21) Appl. No.: 17/776,269

(22) PCT Filed: Nov. 12, 2020
(86) PCT No.: PCT/US2020/060272

§ 371 Date: May 12, 2022

Related U.S. Application Data
(60) Provisional application No. 62/934,054, filed on Nov. 12, 2019.
(60) Provisional application No. 63/000,260, filed on Mar. 26, 2020.

(51) Int. Cl. C12N 9/22; C12N 15/11; C12N 9/78; C12N 15/90; C12N 15/82

(52) U.S. Cl. CPC C12N 9/22; C12N 15/11; C12N 9/78; C12Y 305/04004; C12Y 305/04005; C12N 15/902; C12N 15/8213; C12N 2310/20

(57) ABSTRACT

The present disclosure provides for systems, methods, and compositions for targeting nucleic acids. In particular, the invention provides small Cas proteins and their use in modifying target sequences. In one aspect, the present disclosure provides a non-naturally occurring or engineered system comprising: a Cas protein that comprises a RuvC domain and a HNH domain, and is less than 850 amino acids in size; and a guide sequence capable of forming a complex with the Cas protein and directing the complex to bind to a target sequence.

### CROSS REFERENCE TO RELATED APPLICATIONS

This application claims the benefit of U.S. Provisional Application No. 62/934,054 filed Nov. 12, 2019, and U.S. Provisional Application No. 63/000,260, filed Mar. 26, 2020. The entire contents of the above-identified applications are hereby fully incorporated herein by reference.

### STATEMENT REGARDING FEDERALLY SPONSORED RESEARCH

This invention was made with government support under Grant No. HL141201 awarded by the National Institutes of Health. The government has certain rights in the invention.

### REFERENCE TO AN ELECTRONIC SEQUENCE LISTING

The contents of the electronic sequence listing (BROD-4660WP.ST25.txt”; Size is 227,053 bytes and it was created on Nov. 12, 2020) is herein incorporated by reference in its entirety.

### TECHNICAL FIELD

The subject matter disclosed herein generally relates to systems, methods and compositions used for the control of gene expression involving sequence targeting, such as perturbation of gene transcripts or nucleic acid editing, that may use vector systems related to Clustered Regularly Interspaced Short Palindromic Repeats (CRISPR) and components thereof.

### BACKGROUND

The CRISPR-CRISPR associated (Cas) systems of bacterial and archaeal adaptive immunity are such systems that show extreme diversity of protein composition and genomic loci architecture. There exists a pressing need for alternative and robust systems and techniques for targeting nucleic acids or polynucleotides.

### SUMMARY

In one aspect, the present disclosure provides a non-naturally occurring or engineered system comprising: a Cas protein that comprises a RuvC domain and a HNH domain, and is less than 850 amino acids in size; and a guide sequence capable of forming a complex with the Cas protein and directing the complex to bind to a target sequence.

In some embodiments, the Cas protein is a Type II Cas protein. In some embodiments, the Type II Cas protein is a Type II-B Cas protein. In some embodiments, the Type II Cas protein is a Type II-C Cas protein. In some embodiments, the Type II Cas protein is Cas9 or an ortholog thereof. In some embodiments, the Cas protein is a protein from Table 12. In some embodiments, the Cas protein is from or derived from Gammaproteobacteria bacterium AqS3, Deltaproteobacteria bacterium GWF2_42_12, JGI Metagenome: IMG 3300025323, Nitrospirae bacterium RBG_13_39_12, or Nitrospiraceae bacterium isolate UBA9935. In some embodiments, the composition comprises two or more guide sequences capable of hybridizing to two different target sequences or different regions of a target sequence. In some embodiments, the guide sequence is capable of hybridizing to one or more target sequences in a prokaryotic cell. In some embodiments, the guide sequence is capable of hybridizing to one or more target sequences in a eukaryotic cell. In some embodiments, the Cas protein comprises one or more nuclear localization signals. In some embodiments, the Cas protein comprises two or more nuclear localization signals. In some embodiments, the Cas protein comprises one or more nuclear export signals. In some embodiments, the Cas protein is catalytically inactive. In some embodiments, the Cas protein is a nickase.

In some embodiments, the Cas protein is associated with one or more functional domains. In some embodiments, the one or more functional domains comprises one or more heterologous functional domains. In some embodiments, the one or more functional domains cleaves the target sequence. In some embodiments, the one or more functional domains modifies transcription or translation of the target sequence. In some embodiments, the one or more functional domains comprises one or more transcriptional activation domains. In some embodiments, the one or more transcriptional activation domains comprises VP64. In some embodiments, the one or more functional domains comprises one or more transcriptional repression domains. In some embodiments, the one or more transcriptional repression domains comprises a KRAB domain or a SID domain. In some embodiments, the one or more functional domains comprises one or more nuclease domains. In some embodiments, the one or more nuclease domains comprises Fok1. In some embodiments, the one or more functional domains has one or more of the following activities: methylase activity, demethylase activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity and nucleic acid binding activity. In some embodiments, the composition further comprises a recombination template.

In some embodiments, the recombination template is inserted by homology-directed repair (HDR). In some embodiments, the composition further comprises a tracr RNA. In some embodiments, the Cas protein is a chimeric protein comprising a first fragment from a first Cas protein and a second fragment from a second Cas protein.

In some embodiments, the composition further comprises a nucleotide deaminase or a catalytic domain thereof. In some embodiments, the nucleotide deaminase is an adenosine deaminase. In some embodiments, the nucleotide deaminase is a cytidine deaminase. In some embodiments, the nucleotide deaminase or catalytic domain thereof is covalently or non-covalently linked to the Cas protein or the guide sequence, or is adapted to link thereof after delivered to a cell. In some embodiments, the nucleotide deaminase or catalytic domain thereof has been modified to increase its activity against a DNA-RNA heteroduplex. In some embodiments, the nucleotide deaminase or catalytic domain thereof has been modified to reduce off-target effects. In some embodiments, the composition is capable of modifying one or more nucleotides in the target sequence.

In some embodiments, modification of the one or more nucleotides in the target sequence remedies a disease caused by a G→A or C→T point mutation or a pathogenic SNP. In some embodiments, the disease is cancer, hemophilia, beta-thalassemia, Marfan syndrome, or Wiskott-Aldrich syndrome. In some embodiments, modification of the one or more nucleotides in the target sequence remedies a disease caused by a T→C or A→G point mutation or a pathogenic SNP. In some embodiments, modification of the one or more nucleotides at the target sequence inactivates a gene. In some embodiments, modification of the one or more nucleotides modifies gene product encoded at the target sequence or expression of the gene product. In some embodiments, the composition further comprises a reverse transcriptase or a functional fragment thereof.

In another aspect, the present disclosure provides a non-naturally occurring or engineered composition comprising one or more polynucleotide sequences encoding: a Cas protein that comprises a RuvC domain and a HNH domain, and is less than 900 amino acids in size; and a guide sequence capable of forming a complex with the Cas protein and directing the complex to bind to a target sequence. In some embodiments, the one or more polynucleotide sequences are codon optimized to express in a eukaryote. In some embodiments, the one or more polynucleotide sequences is mRNA. In some embodiments, the one or more polynucleotide sequences further encode a reverse transcriptase or a functional fragment thereof.

In another aspect, the present disclosure provides a vector composition comprising the one or more polynucleotides sequences herein. In some embodiments, the vector composition comprises a first regulatory element operably linked to the polynucleotide sequence encoding the Cas protein; and a second regulatory element operably linked to the polynucleotide sequence encoding the guide sequence. In some embodiments, the first and/or second regulatory element is a promoter. In some embodiments, the promoter is a minimal promoter. In some embodiments, the minimal promoter is Mecp2 promoter, tRNA promoter, or U6 promoter. In some embodiments, is comprised in a single vector. In some embodiments, the one or more vectors comprises viral vectors. In some embodiments, the one or more vectors comprises retroviral, lentiviral, adenoviral, adeno-associated, or Herpes simplex viral vectors.

In another aspect, the present disclosure provides a delivery composition comprising the composition of herein and a delivery vehicle. In some embodiments, the delivery vehicle comprises lipids, sugars, metals, proteins, liposomes, nanoparticles, exosomes, microvesicles, nucleic acid nanoassemblies, a gene gun, an implantable device, or a vector composition. In some embodiments, the delivery vehicle comprises ribonucleoproteins. In another aspect, the present disclosure provides a cell comprising the composition herein. In some embodiments, the cell is a eukaryotic cell, a human or non-human animal cell, a therapeutic T cell, antibody-producing B-cell, a stem cell, or a plant cell.

In another aspect, the present disclosure provides a tissue, organ, or organism comprising the cell herein.

In another aspect, the present disclosure provides a cell product from the cell herein.

In another aspect, the present disclosure provides a method of modifying one or more target sequences, the method comprising contacting the one or more target sequences with a composition herein. In some embodiments, the composition further comprises a recombination template, and wherein modifying the one or more target sequences comprises insertion of the recombination template or a portion thereof. In some embodiments, the one or more target sequences is in a prokaryotic cell. In some embodiments, the one or more target sequences is in a eukaryotic cell. In some embodiments, the one or more target sequences is comprised in a nucleic acid molecule in vitro.

In another aspect, the present disclosure provides a cell obtained from the method herein. In some embodiments, the cell is a eukaryotic cell, a human or non-human animal cell, a therapeutic T cell, antibody-producing B-cell, a stem cell, or a plant cell.

In another aspect, the present disclosure provides a non-human animal or plant comprising the modified cell herein or progeny thereof.

In another aspect, the present disclosure provides a modified cell herein or progeny thereof for use in therapy.

In another aspect, the present disclosure provides a method of treating a disease, disorder, or infection comprising administering an effective amount of the composition herein a subject in need thereof.

In another aspect, the present disclosure provides a method of producing a plant having a modified trait of interest encoded by a gene of interest, the method comprises contacting a plant cell with a composition herein, thereby either modifying or introducing the gene of interest, and regenerating a plant from the plant cell.

In another aspect, the present disclosure provides a method of identifying a trait of interest in a plant, the trait of interest encoded by a gene of interest, the method comprises contacting a plant cell with a composition herein, thereby identifying the gene of interest.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 shows an exemplary Type II-C Cas9.

FIG. 2 shows results of determination of PAM of the exemplary Type II-C Cas9 in FIG. 1.

FIG. 3 shows purification pull down experiments to determine small RNAs associated with the exemplary Cas9 in FIG. 1.

FIG. 4 shows DNA cleavage activity of the exemplary Cas9 in FIG. 1.

FIG. 5 shows the structure of the crRNA and tracrRNA in the form of a complex.

FIG. 6 shows exemplary Type II-B Cas9 proteins.

FIG. 7 shows an exemplary method of identifying and characterizing Cas proteins.

FIG. 8 shows exemplary Cas9-t had interference activity with NGCH PAM.

FIG. 9 shows pulldown of the Cas9-t protein bound to ncRNAs revealed processed CRISPR and tracrRNA.

FIG. 10 shows the cleavage of dsDNA by an exemplary Cas9-t in vitro using an sgRNA.

The figures herein are for illustrative purposes only and are not necessarily drawn to scale.

### DETAILED DESCRIPTION OF THE EXAMPLE EMBODIMENTS

### Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011)

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The term “about” in relation to a reference numerical value and its grammatical equivalents as used herein can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

A protein or nucleic acid derived from a species means that the protein or nucleic acid has a sequence identical to an endogenous protein or nucleic acid or a portion thereof in the species. The protein or nucleic acid derived from the species may be directly obtained from an organism of the species (e.g., by isolation), or may be produced, e.g., by recombination production or chemical synthesis.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

It will be appreciated that the terms Cas enzyme, CRISPR enzyme, CRISPR protein, Cas protein and CRISPR Cas are generally used interchangeably and at all points of reference herein refer by analogy to CRISPR effector proteins further described in this application, unless otherwise apparent.

### Overview

In one aspect, the present disclosure provides compositions, systems and methods for nucleic acid modification. In some embodiments, the compositions and systems herein comprise a sub-set of newly identified Class 2, Type II Cas proteins that are smaller in size than previously discovered Class 2, Type II Cas proteins. In some embodiments, the compositions and systems comprise one or more Type II Cas proteins that are less than 850 amino acids in size and one or more guide sequences. The relatively small sizes of these Cas protein may allow easier engineering, multiplexing, packaging, and delivery, and use as a component in a fusion construct, e.g., fusion with a nucleotide deaminase. In some examples, the Type II Cas proteins are Type II-B Cas 9 or Type II-C Cas 9 proteins. In particular examples, the Cas proteins are Cas 9 proteins described in Table 12.

In another aspect, embodiments disclosed herein include compositions and systems and uses for such Cas proteins including diagnostics, base editing therapeutics and methods of detection. Fusion proteins comprising a small Type II Cas protein herein, and nucleotide deaminase may also be used for base editing. Delivery of the proteins and systems disclosed is also provided, including to a variety of cells and via a variety of particles, vesicles and vectors.

### Systems and Compositions in General

In one aspect, the present disclosure provides for systems and compositions for modification of nucleic acids. In general, the systems or composition may comprise one or more small Cas proteins that comprise at least one RuvC domain and at least one HNH domain. The systems and compositions may further comprise one or more guide sequences. The guide sequences may be capable of hybridizing to a target sequence.

In some examples, the small Cas proteins may be small Type II Cas proteins. In some embodiments, the Type II Cas proteins are Type II-B or Type II-C Cas proteins. In particular examples, the Type II Cas proteins are Type II-B Cas9 or Type II-C Cas9 proteins. In some examples, the Cas 9 protein may be from or derived from Gammaproteobacteria bacterium AqS3, Deltaproteobacteria bacterium GWF2_42_12, JGI Metagenome: IMG 3300025323, Nitrospirae bacterium RBG_13_39_12, Nitrospiraceae bacterium isolate UBA9935, or orthologs thereof. In certain example embodiments, the small Cas proteins may be less than 850 amino acids in size.

### Crispr-Cas Systems in General

In general, a Cas protein (used interchangeably herein with CRISPR protein, CRISPR enzyme, CRISPR-Cas protein, CRISPR-Cas enzyme, or Cas protein) and/or a guide sequence is a component of a CRISPR-Cas system. A CRISPR-Cas system or CRISPR system refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In an engineered system, the direct repeat may encompass naturally-occurring sequences or non-naturally-occurring sequences. The direct repeat is not limited to naturally occurring lengths and sequences. A direct repeat can be 36 nt in length, but a longer or shorter direct repeat can vary. For example, a direct repeat can be 20 nt or longer, such as 30-100 nt or longer. For example, a direct repeat can be, 20 nt, 30 nt, 40 nt, 50 nt, 60 nt, 70 nt, 70 nt, 80 nt, 90 nt, 100 nt or longer in length. In some embodiments, a direct repeat can include synthetic nucleotide sequences inserted between the 5′ and 3′ ends of naturally occurring direct repeats. In certain embodiments, the inserted sequence may be self-complementary, for example, 20%, 30%, 40%, 50%, 60%, 70%, 80%, 90%, or 100% self-complementary. Furthermore, a direct repeat may include insertions of nucleotides such as an aptamer or sequences that bind to an adapter protein (for association with functional domains). In certain embodiments, one end of a direct repeat containing such an insertion is roughly the first half of a short DR and the end is roughly the second half of the short DR.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides. In some embodiments, a target sequence is located in the nucleus or cytoplasm of a cell. In some embodiments, direct repeats may be identified in silico by searching for repetitive motifs that fulfill any or all of the following criteria: 1. found in a 2 Kb window of genomic sequence flanking the type II CRISPR locus; 2. span from 20 to 50 bp; and 3. interspaced by 20 to 50 bp. In some embodiments, 2 of these criteria may be used, for instance 1 and 2, 2 and 3, or 1 and 3. In some embodiments, all 3 criteria may be used.

In some embodiments the terms guide sequence refers to nucleic acid molecules (e.g., guide RNA) capable of guiding Cas proteins to a target locus. In general, a guide sequence (or spacer sequence) is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). In some embodiments, a guide sequence (or spacer sequence) is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. Preferably the guide sequence is 10-40 nucleotides long, such as 20-30 or 20-40 nucleotides long or longer, such as 30 nucleotides long or about 30 nucleotides long. In certain embodiments, the guide sequence is 10-30 nucleotides long, such as 20-30 or 20-40 nucleotides long or longer, such as 30 nucleotides long or about 30 nucleotides long for CRISPR-Cas effectors. In certain embodiments, the guide sequence is 10-30 nucleotides long, such as 20-30 nucleotides long, such as 30 nucleotides long. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

In some CRISPR-Cas systems, the degree of complementarity between a guide sequence and its corresponding target sequence can be about or more than about 50%, 60%, 75%, 80%, 85%, 90%, 95%, 97.5%, 99%, or 100%; a guide or RNA or crRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or crRNA can be less than about 75, 50, 45, 40, 35, 30, 25, 20, 15, 12, or fewer nucleotides in length; and advantageously tracr RNA is 30 or 50 nucleotides in length. However, an aspect of the invention is to reduce off-target interactions, e.g., reduce the guide interacting with a target sequence having low complementarity. Indeed, in the examples, it is shown that the invention involves mutations that result in the CRISPR-Cas system being able to distinguish between target and off-target sequences that have greater than 80% to about 95% complementarity, e.g., 83%-84% or 88-89% or 94-95% complementarity (for instance, distinguishing between a target having 18 nucleotides from an off-target of 18 nucleotides having 1, 2 or 3 mismatches). Accordingly, in the context of the present invention the degree of complementarity between a guide sequence and its corresponding target sequence may be greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In certain embodiments, modulations of cleavage efficiency can be exploited by introduction of mismatches, e.g. 1 or more mismatches, such as 1 or 2 mismatches between spacer sequence and target sequence, including the position of the mismatch along the spacer/target. The more close to the middle (i.e. not 3′ or 5′) for instance a double mismatch is, the more cleavage efficiency may be affected. Accordingly, by choosing mismatch position along the spacer, cleavage efficiency can be modulated. By means of example, if less than 100% cleavage of targets is desired (e.g. in a cell population), 1 or more, such as preferably 2 mismatches between spacer and target sequence may be introduced in the spacer sequences. The more central along the spacer of the mismatch position, the lower the cleavage percentage.

The methods according to the present disclosure as described herein comprehend inducing one or more nucleotide modifications in a eukaryotic cell (in vitro, i.e. in an isolated eukaryotic cell) as herein discussed comprising delivering to cell a vector as herein discussed. The mutation(s) can include the introduction, deletion, or substitution of one or more nucleotides at each target sequence of cell(s) via the guide(s) RNA(s) or sgRNA(s). The mutations can include the introduction, deletion, or substitution of 1-75 nucleotides at each target sequence of said cell(s) via the guide(s) RNA(s). The mutations can include the introduction, deletion, or substitution of 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s). The mutations can include the introduction, deletion, or substitution of 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s). The mutations include the introduction, deletion, or substitution of 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s). The mutations can include the introduction, deletion, or substitution of 20, 21, 22, 23, 24, 25, 26, 27, 28, 29, 30, 35, 40, 45, 50, or 75 nucleotides at each target sequence of said cell(s) via the guide(s) RNA(s). The mutations can include the introduction, deletion, or substitution of 40, 45, 50, 75, 100, 200, 300, 400 or 500 nucleotides at each target sequence of said cell(s) via the guide(s) RNA(s).

For minimization of toxicity and off-target effect, it will be important to control the concentration of Cas mRNA or protein and guide RNA delivered. Optimal concentrations of Cas mRNA or protein and guide RNA can be determined by testing different concentrations in a cellular or non-human eukaryote animal model and using deep sequencing the analyze the extent of modification at potential off-target genomic loci.

Typically, in the context of an endogenous CRISPR system, formation of a CRISPR complex (comprising a guide sequence hybridized to a target sequence and complexed with one or more Cas proteins) results in cleavage in or near (e.g. within 1, 2, 3, 4, 5, 6, 7, 8, 9, 10, 20, 50, or more base pairs from) the target sequence, but may depend on for instance secondary structure, in particular in the case of RNA targets. In some cases, in the context of an endogenous CRISPR system, formation of a CRISPR complex (comprising a guide sequence hybridized to a target sequence and complexed with one or more Cas proteins) results in cleavage of one or both strands (if applicable) in or near (e.g. within 1, 2, 3, 4, 5, 6, 7, 8, 9, 10, 20, 50, or more base pairs from) the target sequence.

In particularly preferred embodiments according to the present disclosure, the guide RNA (capable of guiding Cas to a target locus) may comprise (1) a guide sequence capable of hybridizing to a target locus (a polynucleotide target locus, such as an RNA target locus) in the eukaryotic cell; (2) a direct repeat (DR) sequence) which reside in a single RNA, i.e. an sgRNA (arranged in a 5′ to 3′ orientation) or crRNA.

With respect to general information on CRISPR-Cas Systems, components thereof, and delivery of such components, including methods, materials, delivery vehicles, vectors, particles, AAV, and making and using thereof, including as to amounts and formulations, all useful in the practice of the instant invention, reference is made to: U.S. Pat. Nos. 8,999,641, 8,993,233, 8,945,839, 8,932,814, 8,906,616, 8,895,308, 8,889,418, 8,889,356, 8,871,445, 8,865,406, 8,795,965, 8,771,945 and 8,697,359; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); European Patents EP 2784162 B1 and EP 2771468 B1; European Patent Applications EP 2771468 (EP13818570.7), EP 2764103 (EP13824232.6), and EP 2784162 (EP14170383.5); and PCT Patent Publications PCT Patent Publications WO 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), WO 2014/093701 (PCT/US2013/074800), WO 2014/018423 (PCT/US2013/051418), WO 2014/204723 (PCT/US2014/041790), WO 2014/204724 (PCT/US2014/041800), WO 2014/204725 (PCT/US2014/041803), WO 2014/204726 (PCT/US2014/041804), WO 2014/204727 (PCT/US2014/041806), WO 2014/204728 (PCT/US2014/041808), WO 2014/204729 (PCT/US2014/041809). Reference is also made to U.S. provisional patent applications 61/758,468; 61/802,174; 61/806,375; 61/814,263; 61/819,803 and 61/828,130, filed on Jan. 30, 2013; Mar. 15, 2013; Mar. 28, 2013; Apr. 20, 2013; May 6, 2013 and May 28, 2013 respectively. Reference is also made to U.S. provisional patent application 61/836,123, filed on Jun. 17, 2013. Reference is additionally made to U.S. provisional patent applications 61/835,931, 61/835,936, 61/836,127, 61/836,101, 61/836,080 and 61/835,973, each filed Jun. 17, 2013. Further reference is made to U.S. provisional patent applications 61/862,468 and 61/862,355 filed on Aug. 5, 2013; 61/871,301 filed on Aug. 28, 2013; 61/960,777 filed on Sep. 25, 2013 and 61/961,980 filed on Oct. 28, 2013. Reference is yet further made to: PCT Patent applications Nos: PCT/US2014/041803, PCT/US2014/041800, PCT/US2014/041809, PCT/US2014/041804 and PCT/US2014/041806, each filed Jun. 10, 2014 6/10/14; PCT/US2014/041808 filed Jun. 11, 2014; and PCT/US2014/62558 filed Oct. 28, 2014, and US Provisional Patent Applications Ser. Nos. 61/915,150, 61/915,301, 61/915,267 and 61/915,260, each filed Dec. 12, 2013; 61/757,972 and 61/768,959, filed on Jan. 29, 2013 and Feb. 25, 2013; 61/835,936, 61/836,127, 61/836,101, 61/836,080, 61/835,973, and 61/835,931, filed Jun. 17, 2013; 62/010,888 and 62/010,879, both filed Jun. 11, 2014; 62/010,329 and 62/010,441, each filed Jun. 10, 2014; 61/939,228 and 61/939,242, each filed Feb. 12, 2014; 61/980,012, filed Apr. 15, 2014; 62/038,358, filed Aug. 17, 2014; 62/054,490, 62/055,484, 62/055,460 and 62/055,487, each filed Sep. 25, 2014; and 62/069,243, filed Oct. 27, 2014. Reference is also made to U.S. provisional patent applications Nos. 62/055,484, 62/055,460, and 62/055,487, filed Sep. 25, 2014; U.S. provisional patent application 61/980,012, filed Apr. 15, 2014; and U.S. provisional patent application 61/939,242 filed Feb. 12, 2014. Reference is made to PCT application designating, inter alia, the United States, application No. PCT/US14/41806, filed Jun. 10, 2014. Reference is made to U.S. provisional patent application 61/930,214 filed on Jan. 22, 2014. Reference is made to U.S. provisional patent applications 61/915,251; 61/915,260 and 61/915,267, each filed on Dec. 12, 2013. Reference is made to US provisional patent application U.S. Ser. No. 61/980,012 filed Apr. 15, 2014. Reference is made to PCT application designating, inter alia, the United States, application No. PCT/US14/41806, filed Jun. 10, 2014. Reference is made to U.S. provisional patent application 61/930,214 filed on Jan. 22, 2014. Reference is made to U.S. provisional patent applications 61/915,251; 61/915,260 and 61/915,267, each filed on Dec. 12, 2013.

Mention is also made of U.S. application 62/091,455, filed, 12 Dec. 14, PROTECTED GUIDE RNAS (PGRNAS); U.S. application 62/096,708, 24 Dec. 14, PROTECTED GUIDE RNAS (PGRNAS); U.S. application 62/091,462, 12 Dec. 14, DEAD GUIDES FOR CRISPR TRANSCRIPTION FACTORS; U.S. application 62/096,324, 23 Dec. 14, DEAD GUIDES FOR CRISPR TRANSCRIPTION FACTORS; U.S. application 62/091,456, 12 Dec. 14, ESCORTED AND FUNCTIONALIZED GUIDES FOR CRISPR-CAS SYSTEMS; U.S. application 62/091,461, 12 Dec. 14, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19 Dec. 14, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24 Dec. 14, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 14, RNA-TARGETING SYSTEM; U.S. application 62/096,656, 24 Dec. 14, CRISPR HAVING OR ASSOCIATED WITH DESTABILIZATION DOMAINS; U.S. application 62/096,697, 24 Dec. 14, CRISPR HAVING OR ASSOCIATED WITH AAV; U.S. application 62/098,158, 30 Dec. 14, ENGINEERED CRISPR COMPLEX INSERTIONAL TARGETING SYSTEMS; U.S. application 62/151,052, 22 Apr. 15, CELLULAR TARGETING FOR EXTRACELLULAR EXOSOMAL REPORTING; U.S. application 62/054,490, 24 Sep. 14, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 62/055,484, 25 Sep. 14, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 14, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24 Sep. 14, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23 Oct. 14, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/054,675, 24 Sep. 14, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24 Sep. 14, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 14, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 14, MULTIFUNCTIONAL-CRISPR COMPLEXES AND/OR OPTIMIZED ENZYME LINKED FUNCTIONAL-CRISPR COMPLEXES; U.S. application 62/087,475, 4 Dec. 14, FUNCTIONAL SCREENING WITH OPTIMIZED FUNCTIONAL CRISPR-CAS SYSTEMS; U.S. application 62/055,487, 25 Sep. 14, FUNCTIONAL SCREENING WITH OPTIMIZED FUNCTIONAL CRISPR-CAS SYSTEMS; U.S. application 62/087,546, 4 Dec. 14, MULTIFUNCTIONAL CRISPR COMPLEXES AND/OR OPTIMIZED ENZYME LINKED FUNCTIONAL-CRISPR COMPLEXES; and U.S. application 62/098,285, 30 Dec. 14, CRISPR MEDIATED IN VIVO MODELING AND GENETIC SCREENING OF TUMOR GROWTH AND METASTASIS.

Also with respect to general information on CRISPR-Cas Systems, mention is made of the following (also hereby incorporated herein by reference):
Multiplex genome engineering using CRISPR/Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013);
RNA-guided editing of bacterial genomes using CRISPR-Cas systems. Jiang W., Bikard D., Cox D., Zhang F, Marraffini L A. Nat Biotechnol March; 31(3):233-9 (2013);
One-Step Generation of Mice Carrying Mutations in Multiple Genes by CRISPR/Cas-Mediated Genome Engineering. Wang H., Yang H., Shivalila C S., Dawlaty M M., Cheng A W., Zhang F., Jaenisch R. Cell May 9; 153(4):910-8 (2013);
Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. August 22; 500(7463):472-6. doi: 10.1038/Nature12466. Epub 2013 Aug. 23 (2013);
Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674(13)01015-5 (2013-A);
DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi:10.1038/nbt.2647 (2013);
Genome engineering using the CRISPR-Cas9 system. Ran, F A., Hsu, P D., Wright, J., Agarwala, V., Scott, D A., Zhang, F. Nature Protocols November; 8(11):2281-308 (2013-B);
Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013). [Epub ahead of print];
Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27, 156(5):935-49 (2014);
Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. April 20. doi: 10.1038/nbt.2889 (2014);
CRISPR-Cas9 Knockin Mice for Genome Editing and Cancer Modeling. Platt R J, Chen S, Zhou Y, Yim M J, Swiech L, Kempton H R, Dahlman J E, Parnas O, Eisenhaure T M, Jovanovic M, Graham D B, Jhunjhunwala S, Heidenreich M, Xavier R J, Langer R, Anderson D G, Hacohen N, Regev A, Feng G, Sharp P A, Zhang F. Cell 159(2): 440-455 DOI: 10.1016/j.cell.2014.09.014(2014);
Development and Applications of CRISPR-Cas9 for Genome Engineering, Hsu P D, Lander E S, Zhang F., Cell. June 5; 157(6):1262-78 (2014).
Genetic screens in human cells using the CRISPR/Cas9 system, Wang T, Wei J J, Sabatini D M, Lander E S., Science. January 3; 343(6166): 80-84. doi:10.1126/science.1246981 (2014);
Rational design of highly active sgRNAs for CRISPR-Cas9-mediated gene inactivation, Doench J G, Hartenian E, Graham D B, Tothova Z, Hegde M, Smith I, Sullender M, Ebert B L, Xavier R J, Root D E., (published online 3 Sep. 2014) Nat Biotechnol. December; 32(12):1262-7 (2014);
In vivo interrogation of gene function in the mammalian brain using CRISPR-Cas9, Swiech L, Heidenreich M, Banerjee A, Habib N, Li Y, Trombetta J, Sur M, Zhang F., (published online 19 Oct. 2014) Nat Biotechnol. January; 33(1):102-6 (2015);
Genome-scale transcriptional activation by an engineered CRISPR-Cas9 complex, Konermann S, Brigham M D, Trevino A E, Joung J, Abudayyeh O O, Barcena C, Hsu P D, Habib N, Gootenberg J S, Nishimasu H, Nureki O, Zhang F., Nature. January 29; 517(7536):583-8 (2015).
A split-Cas9 architecture for inducible genome editing and transcription modulation, Zetsche B, Volz S E, Zhang F., (published online 2 Feb. 2015) Nat Biotechnol. February; 33(2):139-42 (2015);
Genome-wide CRISPR Screen in a Mouse Model of Tumor Growth and Metastasis, Chen S, Sanjana N E, Zheng K, Shalem O, Lee K, Shi X, Scott D A, Song J, Pan J Q, Weissleder R, Lee H, Zhang F, Sharp P A. Cell 160, 1246-1260, Mar. 12, 2015 (multiplex screen in mouse), and
In vivo genome editing using Staphylococcus aureus Cas9, Ran F A, Cong L, Yan W X, Scott D A, Gootenberg J S, Kriz A J, Zetsche B, Shalem O, Wu X, Makarova K S, Koonin E V, Sharp P A, Zhang F., (published online 1 Apr. 2015), Nature. April 9; 520(7546):186-91 (2015).
Shalem et al., “High-throughput functional genomics using CRISPR-Cas9,” Nature Reviews Genetics 16, 299-311 (May 2015).
Xu et al., “Sequence determinants of improved CRISPR sgRNA design,” Genome Research 25, 1147-1157 (August 2015).
Parnas et al., “A Genome-wide CRISPR Screen in Primary Immune Cells to Dissect Regulatory Networks,” Cell 162, 675-686 (Jul. 30, 2015).
Ramanan et al., CRISPR/Cas9 cleavage of viral DNA efficiently suppresses hepatitis B virus,” Scientific Reports 5:10833. doi: 10.1038/srep10833 (Jun. 2, 2015)
Nishimasu et al., Crystal Structure of Staphylococcus aureus Cas9,” Cell 162, 1113-1126 (Aug. 27, 2015)
Zetsche et al. (2015), “Cpf1 is a single RNA-guided endonuclease of a class 2 CRISPR-Cas system,” Cell 163, 759-771 (Oct. 22, 2015) doi: 10.1016/j.cell.2015.09.038. Epub Sep. 25, 2015
Shmakov et al. (2015), “Discovery and Functional Characterization of Diverse Class 2 CRISPR-Cas Systems,” Molecular Cell 60, 385-397 (Nov. 5, 2015) doi: 10.1016/j.molcel.2015.10.008. Epub Oct. 22, 2015
Dahlman et al., “Orthogonal gene control with a catalytically active Cas9 nuclease,” Nature Biotechnology 33, 1159-1161 (November, 2015)
Gao et al, “Engineered Cpf1 Enzymes with Altered PAM Specificities,” bioRxiv 091611; doi: dx.doi.org/10.1101/091611 Epub Dec. 4, 2016
Smargon et al. (2017), “Cas13b Is a Type VI-B CRISPR-Associated RNA-Guided RNase Differentially Regulated by Accessory Proteins Csx27 and Csx28,” Molecular Cell 65, 618-630 (Feb. 16, 2017) doi: 10.1016/j.molcel.2016.12.023. Epub Jan. 5, 2017 each of which is incorporated herein by reference, may be considered in the practice of the instant invention, and discussed briefly below:
Cong et al. engineered type II CRISPR-Cas systems for use in eukaryotic cells based on both Streptococcus thermophilus Cas9 and also Streptococcus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR-Cas system can be further improved to increase its efficiency and versatility.
Jiang et al. used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA:Cas9-directed cleavage at the targeted genomic site to kill unmutated cells and circumvents the need for selectable markers or counter-selection systems. The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.
Wang et al. (2013) used the CRISPR/Cas system for the one-step generation of mice carrying mutations in multiple genes which were traditionally generated in multiple steps by sequential recombination in embryonic stem cells and/or time-consuming intercrossing of mice with a single mutation. The CRISPR/Cas system will greatly accelerate the in vivo study of functionally redundant genes and of epi static gene interactions.
Konermann et al. (2013) addressed the need in the art for versatile and robust technologies that enable optical and chemical modulation of DNA-binding domains based CRISPR Cas9 enzyme and also Transcriptional Activator Like Effectors
Ran et al. (2013-A) described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a guide sequence, which can tolerate certain mismatches to the DNA target and thereby promote undesired off-target mutagenesis. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity.
Hsu et al. (2013) characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and sgRNA can be titrated to minimize off-target modification. Additionally, to facilitate mammalian genome engineering applications, the authors reported providing a web-based software tool to guide the selection and validation of target sequences as well as off-target analyses.
Ran et al. (2013-B) described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.
Shalem et al. described a new way to interrogate gene function on a genome-wide scale. Their studies showed that delivery of a genome-scale CRISPR-Cas9 knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hits NF2, CUL3, TADA2B, and TADA1. The authors observed a high level of consistency between independent guide RNAs targeting the same gene and a high rate of hit confirmation, and thus demonstrated the promise of genome-scale screening with Cas9.
Nishimasu et al. reported the crystal structure of Streptococcus pyogenes Cas9 in complex with sgRNA and its target DNA at 2.5 A° resolution. The structure revealed a bilobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.
Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences; thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive pairing with target DNA is required for cleavage.
Platt et al. established a Cre-dependent Cas9 knockin mouse. The authors demonstrated in vivo as well as ex vivo genome editing using adeno-associated virus (AAV)-, lentivirus-, or particle-mediated delivery of guide RNA in neurons, immune cells, and endothelial cells.
Hsu et al. (2014) is a review article that discusses generally CRISPR-Cas9 history from yogurt to genome editing, including genetic screening of cells.
Wang et al. (2014) relates to a pooled, loss-of-function genetic screening approach suitable for both positive and negative selection that uses a genome-scale lentiviral single guide RNA (sgRNA) library.
Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.
Swiech et al. demonstrate that AAV-mediated SpCas9 genome editing can enable reverse genetic studies of gene function in the brain.
Konermann et al. (2015) discusses the ability to attach multiple effector domains, e.g., transcriptional activator, functional and epigenomic regulators at appropriate positions on the guide such as stem or tetraloop with and without linkers.
Zetsche et al. demonstrates that the Cas9 enzyme can be split into two and hence the assembly of Cas9 for activation can be controlled.
Chen et al. relates to multiplex screening by demonstrating that a genome-wide in vivo CRISPR-Cas9 screen in mice reveals genes regulating lung metastasis.
Ran et al. (2015) relates to SaCas9 and its ability to edit genomes and demonstrates that one cannot extrapolate from biochemical assays. Shalem et al. (2015) described ways in which catalytically inactive Cas9 (dCas9) fusions are used to synthetically repress (CRISPRi) or activate (CRISPRa) expression, showing. advances using Cas9 for genome-scale screens, including arrayed and pooled screens, knockout approaches that inactivate genomic loci and strategies that modulate transcriptional activity.
Shalem et al. (2015) described ways in which catalytically inactive Cas9 (dCas9) fusions are used to synthetically repress (CRISPRi) or activate (CRISPRa) expression, showing. advances using Cas9 for genome-scale screens, including arrayed and pooled screens, knockout approaches that inactivate genomic loci and strategies that modulate transcriptional activity.
Xu et al. (2015) assessed the DNA sequence features that contribute to single guide RNA (sgRNA) efficiency in CRISPR-based screens. The authors explored efficiency of CRISPR/Cas9 knockout and nucleotide preference at the cleavage site. The authors also found that the sequence preference for CRISPRi/a is substantially different from that for CRISPR/Cas9 knockout.
Parnas et al. (2015) introduced genome-wide pooled CRISPR-Cas9 libraries into dendritic cells (DCs) to identify genes that control the induction of tumor necrosis factor (Tnf) by bacterial lipopolysaccharide (LPS). Known regulators of Tlr4 signaling and previously unknown candidates were identified and classified into three functional modules with distinct effects on the canonical responses to LPS.
Ramanan et al (2015) demonstrated cleavage of viral episomal DNA (cccDNA) in infected cells. The HBV genome exists in the nuclei of infected hepatocytes as a 3.2 kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA.
Nishimasu et al. (2015) reported the crystal structures of SaCas9 in complex with a single guide RNA (sgRNA) and its double-stranded DNA targets, containing the 5′-TTGAAT-3′ PAM and the 5′-TTGGGT-3′ PAM. A structural comparison of SaCas9 with SpCas9 highlighted both structural conservation and divergence, explaining their distinct PAM specificities and orthologous sgRNA recognition.

Also, “Dimeric CRISPR RNA-guided Fok1 nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided Fok1 Nucleases that recognize extended sequences and can edit endogenous genes with high efficiencies in human cells. In addition, mention is made of PCT application PCT/US14/70057, Attorney Reference 47627.99.2060 and BI-2013/107 entitled “DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS (claiming priority from one or more or all of US provisional patent applications: 62/054,490, filed Sep. 24, 2014; 62/010,441, filed Jun. 10, 2014; and 61/915,118, 61/915,215 and 61/915,148, each filed on Dec. 12, 2013) (“the Particle Delivery PCT”), incorporated herein by reference, with respect to a method of preparing an sgRNA-and-Cas9 protein containing particle comprising admixing a mixture comprising an sgRNA and Cas9 protein (and optionally HDR template) with a mixture comprising or consisting essentially of or consisting of surfactant, phospholipid, biodegradable polymer, lipoprotein and alcohol; and particles from such a process. For example, wherein Cas9 protein and sgRNA were mixed together at a suitable, e.g., 3:1 to 1:3 or 2:1 to 1:2 or 1:1 molar ratio, at a suitable temperature, e.g., 15-30C, e.g., 20-25C, e.g., room temperature, for a suitable time, e.g., 15-45, such as 30 minutes, advantageously in sterile, nuclease free buffer, e.g., 1×PBS. Separately, particle components such as or comprising: a surfactant, e.g., cationic lipid, e.g., 1,2-dioleoyl-3-trimethylammonium-propane (DOTAP); phospholipid, e.g., dimyristoylphosphatidylcholine (DMPC); biodegradable polymer, such as an ethylene-glycol polymer or PEG, and a lipoprotein, such as a low-density lipoprotein, e.g., cholesterol were dissolved in an alcohol, advantageously a C₁₋₆ alkyl alcohol, such as methanol, ethanol, isopropanol, e.g., 100% ethanol. The two solutions were mixed together to form particles containing the Cas9-sgRNA complexes. Accordingly, sgRNA may be pre-complexed with the Cas9 protein, before formulating the entire complex in a particle. Formulations may be made with a different molar ratio of different components known to promote delivery of nucleic acids into cells (e.g. 1,2-dioleoyl-3-trimethylammonium-propane (DOTAP), 1,2-ditetradecanoyl-sn-glycero-3-phosphocholine (DMPC), polyethylene glycol (PEG), and cholesterol) For example DOTAP:DMPC:PEG:Cholesterol Molar Ratios may be DOTAP 100, DMPC 0, PEG 0, Cholesterol 0; or DOTAP 90, DMPC 0, PEG 10, Cholesterol 0; or DOTAP 90, DMPC 0, PEG 5, Cholesterol 5. DOTAP 100, DMPC 0, PEG 0, Cholesterol 0. That application accordingly comprehends admixing sgRNA, Cas9 protein and components that form a particle; as well as particles from such admixing. Aspects of the instant invention can involve particles; for example, particles using a process analogous to that of the Particle Delivery PCT, e.g., by admixing a mixture comprising crRNA and/or CRISPR-Cas as in the instant invention and components that form a particle, e.g., as in the Particle Delivery PCT, to form a particle and particles from such admixing (or, of course, other particles involving crRNA and/or CRISPR-Cas as in the instant invention).

### Multiplex Targeting Approach

The Cas proteins herein can employ more than one guide molecules without losing activity. This may enable the use of the Cas proteins, CRISPR-Cas systems or complexes as defined herein for targeting multiple targets (e.g., DNA targets), genes or gene loci, with a single enzyme, system or complex as defined herein. The guide molecules may be tandemly arranged, optionally separated by a nucleotide sequence such as a direct repeat as defined herein. The position of the different guide molecules is the tandem does not influence the activity.

In any of the described methods the complex may be delivered with multiple guides for multiplexed use. In any of the described methods more than one protein(s) may be used. In some examples, one Cas protein may be delivered with multiple guides, e.g., at least 2, at least 5, at least 10, at least 15, at least 20, at least 30, at least 40, at least 50, at least 60, at least 70, at least 80, at least 90, at least 100, at least 120, at least 140, at least 160, at least 180, at least 200, at least 220, at least 240, at least 260, at least 280, at least 300, at least 350, at least 400, or at least 500 guides. In some examples, a system herein may comprise a Cas protein and multiple guides, e.g., at least 2, at least 5, at least 10, at least 15, at least 20, at least 30, at least 40, at least 50, at least 60, at least 70, at least 80, at least 90, at least 100, at least 120, at least 140, at least 160, at least 180, at least 200, at least 220, at least 240, at least 260, at least 280, at least 300, at least 350, at least 400, or at least 500 guides.

The Cas protein may form part of a CRISPR system or complex, which further comprises tandemly arranged guide RNAs (gRNAs) comprising a series of 2, 3, 4, 5, 6, 7, 8, 9, 10, 15, 25, 25, 30, or more than 30 guide sequences, each capable of specifically hybridizing to a target sequence in a genomic locus of interest in a cell. In some embodiments, the functional Cas CRISPR system or complex binds to the multiple target sequences. In some embodiments, the functional CRISPR system or complex may edit the multiple target sequences, e.g., the target sequences may comprise a genomic locus, and in some embodiments, there may be an alteration of gene expression. In some embodiments, the functional CRISPR system or complex may comprise further functional domains. In some embodiments, the composition comprises two or more guide sequences capable of hybridizing to two different target sequences or different regions of a target sequence.

In some embodiments, the invention provides a method for altering or modifying expression of multiple gene products. The method may comprise introducing into a cell containing said target nucleic acids, e.g., DNA molecules, or containing and expressing target nucleic acid, e.g., DNA molecules; for instance, the target nucleic acids may encode gene products or provide for expression of gene products (e.g., regulatory sequences). In some general embodiments, the Cas enzyme used for multiplex targeting is associated with one or more functional domains. In some more specific embodiments, the CRISPR enzyme used for multiplex targeting is a deadCas as defined herein elsewhere. In some embodiments, each of the guide sequence is at least 16, 17, 18, 19, 20, 25 nucleotides, or between 16-30, or between 16-25, or between 16-20 nucleotides in length. Examples of multiplex genome engineering using CRISPR effector proteins are provided in Cong et al. (Science February 15; 339(6121):819-23 (2013) and other publications cited herein.

In any of the described methods the strand break may be a single strand break or a double strand break. In preferred embodiments the double strand break may refer to the breakage of two sections of RNA, such as the two sections of RNA formed when a single strand RNA molecule has folded onto itself or putative double helices that are formed with an RNA molecule which contains self-complementary sequences allows parts of the RNA to fold and pair with itself.

Provided herein are engineered polynucleotide sequences that can direct the activity of a CRISPR protein to multiple targets using a single crRNA. The engineered polynucleotide sequences, also referred to as multiplexing polynucleotides, can include two or more direct repeats interspersed with two or more guide sequences. More specifically, the engineered polynucleotide sequences can include a direct repeat sequence having one or more mutations relative to the corresponding wild type direct repeat sequence. The engineered polynucleotide can be configured, for example, as: 5′ DR1-G1-DR2-G2 3′. In some embodiments, the engineered polynucleotide can be configured to include three, four, five, or more additional direct repeat and guide sequences, for example: 5′ DR1-G1-DR2-G2-DR3-G3 3′, 5″ DR1-G1-DR2-G2-DR3-G3-DR4-G4 3′, or 5′ DR1-G1-DR2-G2-DR3-G3-DR4-G4-DR5-G5 3′.

Regardless of the number of direct repeat sequences, the direct repeat sequences differ from one another. Thus, DR1 can be a wild type sequence and DR2 can include one or more mutations relative to the wild type sequence in accordance with the disclosure provided herein regarding direct repeats for Cas orthologs. The guide sequences can also be the same or different. In some embodiments, the guide sequences can bind to different nucleic acid targets, for example, nucleic acids encoding different polypeptides. The multiplexing polynucleotides can be as described, for example, at [0039]-[0072] in U.S. Application 62/780,748 entitled “CRISPR Cpf1 Direct Repeat Variants” and filed Dec. 17, 2018, incorporated herein in its entirety by reference.

### Type II Cas Proteins

The Cas protein (used interchangeably herein with “Cas protein”, “Cas effector”) may include Cas proteins that have at least one RuvC domain and at least one HNH domain. The Cas protein may have a RuvC-like domain that contains an inserted HNH domain. The Cas proteins may be Class 2 Type II Cas proteins.

In some examples, the Cas protein is Cas9. In some embodiments, Cas9 is a crRNA-dependent endonuclease that contains two unrelated nuclease domains, RuvC and HNH, which are responsible for cleavage of the displaced (non-target) and target DNA strands, respectively, in the crRNA-target DNA complex. Cas9 may be a polypeptide or fragment thereof having at least about 85% amino acid identity to NCBI Accession No. NP 269215 and having RNA binding activity, DNA binding activity, and/or DNA cleavage activity (e.g., endonuclease or nickase activity). “Cas9 function” can be defined by any of a number of assays including, but not limited to, fluorescence polarization-based nucleic acid bind assays, fluorescence polarization-based strand invasion assays, transcription assays, EGFP disruption assays, DNA cleavage assays, and/or Surveyor assays, for example, as described herein. By “Cas 9 nucleic acid molecule” is meant a polynucleotide encoding a Cas9 polypeptide or fragment thereof. An exemplary Cas9 nucleic acid molecule sequence is provided at NCBI Accession No. NC_002737. In some embodiments, disclosed herein are inhibitors of Cas9, e.g., naturally occurring Cas9 in S. pyogenes (SpCas9) or S. aureus (SaCas9), or variants thereof. Cas9 recognizes foreign DNA using Protospacer Adjacent Motif (PAM) sequence and the base pairing of the target DNA by the guide RNA (gRNA). The relative ease of inducing targeted strand breaks at any genomic loci by Cas9 has enabled efficient genome editing in multiple cell types and organisms. Cas9 derivatives can also be used as transcriptional activators/repressors.

In some embodiments, the Cas protein is Type II-A Cas protein. A Type II-A Cas protein may be a Cas protein of a CRISPR-Cas system that comprises Cas9, Cas1, Cas2, and Csn2.

In some embodiments, the Cas protein is Type II-B Cas protein. A Type II-B Cas protein may be a Cas protein of a CRISPR-Cas system that comprises Cas9, Cas1, Cas2, and Cas4.

In some embodiments, the Cas protein is Type II-C Cas protein. A Type II-C Cas protein may be a Cas protein of a CRISPR-Cas system that comprises Cas9, Cas1, Cas2, but not Csn2 or Cas4.

In some embodiments, the Cas protein is less than 1000 amino acids in size. For example, the Cas protein may be less than 950, less than 900, less than 890, less than 880, less than 870, less than 860, less than 850, less than 840, less than 830, less than 820, less than 810, less than 800, less than 790, less than 780, less than 770, less than 760, less than 750, less than 700, less than 650, or less than 600 amino acids in size. In some examples, the Cas protein is less than 850 amino acids in size. As used herein, small Cas9 proteins are also referred to as Cas9-t. In some examples, Cas9-t include Cas9 that have less than 850 amino acids in size.

The systems and methods herein may be used to introduce one or more mutations in nucleic acids. The mutation(s) can include the introduction, deletion, or substitution of one or more nucleotides at each target sequence of cell(s) via the guide(s) RNA(s) or sgRNA(s) or crRNA(s). The mutations can include the introduction, deletion, or substitution of 1-75 nucleotides at each target sequence of said cell(s) via the guide(s) RNA(s) or sgRNA(s) or crRNA(s). The mutations can include the introduction, deletion, or substitution of 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s) or crRNA(s). The mutations can include the introduction, deletion, or substitution of 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s) or crRNA(s). The mutations include the introduction, deletion, or substitution of 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s) or crRNA(s). The mutations can include the introduction, deletion, or substitution of 20, 21, 22, 23, 24, 25, 26, 27, 28, 29, 30, 35, 40, 45, 50, or 75 nucleotides at each target sequence of said cell(s) via the guide(s) RNA(s) or sgRNA(s) or crRNA(s). The mutations can include the introduction, deletion, or substitution of 40, 45, 50, 75, 100, 200, 300, 400 or 500 nucleotides at each target sequence of said cell(s) via the guide(s) RNA(s) or sgRNA(s) or crRNAs.

For minimization of toxicity and off-target effect, it will be important to control the concentration of Cas mRNA and guide RNA delivered. Optimal concentrations of Cas mRNA and guide RNA can be determined by testing different concentrations in a cellular or non-human eukaryote animal model and using deep sequencing the analyze the extent of modification at potential off-target genomic loci. Guide sequences and strategies to minimize toxicity and off-target effects can be as in WO 2014/093622 (PCT/US2013/074667); or, via mutation as herein.

In some embodiments, the Cas proteins may have nucleic acid cleavage activity. The Cas proteins may have RNA binding and DNA cleaving function. In some embodiments, Cas may direct cleavage of one or two nucleic acid strands at the location of or near a target sequence, such as within the target sequence and/or within the complement of the target sequence or at sequences associated with the target sequence, e.g., within about 1, 2, 3, 4, 5, 6, 7, 8, 9, 10, 15, 20, 25, 50, 100, 200, 500, or more base pairs from the first or last nucleotide of a target sequence. In some embodiments, the Cas protein may direct more than one cleavage (such as one, two three, four, five, or more cleavages) of one or two strands within the target sequence and/or within the complement of the target sequence or at sequences associated with the target sequence and/or within about 1, 2, 3, 4, 5, 6, 7, 8, 9, 10, 15, 20, 25, 50, 100, 200, 500, or more base pairs from the first or last nucleotide of a target sequence. In some embodiments, the cleavage may be blunt, i.e., generating blunt ends. In some embodiments, the cleavage may be staggered, i.e., generating sticky ends. In some embodiments, a vector encodes a nucleic acid-targeting Cas protein that may be mutated with respect to a corresponding wild-type enzyme such that the mutated nucleic acid-targeting Cas protein lacks the ability to cleave one or two strands of a target polynucleotide containing a target sequence, e.g., alteration or mutation in a RuvC or HNH domain to produce a mutated Cas substantially lacking all DNA cleavage activity, e.g., the DNA cleavage activity of the mutated enzyme is about no more than 25%, 10%, 5%, 1%, 0.1%, 0.01%, or less of the nucleic acid cleavage activity of the non-mutated form of the enzyme; an example can be when the nucleic acid cleavage activity of the mutated form is nil or negligible as compared with the non-mutated form. By derived, Applicants mean that the derived enzyme is largely based, in the sense of having a high degree of sequence homology with, a wildtype enzyme, but that it has been mutated (modified) in some way as known in the art or as described herein.

Typically, in the context of an endogenous nucleic acid-targeting system, formation of a nucleic acid-targeting complex (comprising a guide RNA or crRNA hybridized to a target sequence and complexed with one or more nucleic acid-targeting effector proteins) results in cleavage of DNA strand(s) in or near (e.g., within 1, 2, 3, 4, 5, 6, 7, 8, 9, 10, 20, 50, or more base pairs from) the target sequence. As used herein the term “sequence(s) associated with a target locus of interest” refers to sequences near the vicinity of the target sequence (e.g. within 1, 2, 3, 4, 5, 6, 7, 8, 9, 10, 20, 50, or more base pairs from the target sequence, wherein the target sequence is comprised within a target locus of interest).

The (i) Cas9 or nucleic acid molecule(s) encoding it or (ii) crRNA can be delivered separately; and advantageously at least one or both of one of (i) and (ii), e.g., an assembled complex is delivered via a particle or nanoparticle complex. The Cas protein mRNA can be delivered prior to the guide RNA or crRNA to give time for nucleic acid-targeting effector protein to be expressed. The Cas protein mRNA might be administered 1-12 hours (preferably around 2-6 hours) prior to the administration of guide RNA or crRNA. Alternatively, the Cas protein mRNA and guide RNA or crRNA can be administered together. Advantageously, a second booster dose of guide RNA or crRNA can be administered 1-12 hours (preferably around 2-6 hours) after the initial administration of Cas protein mRNA+guide RNA. Additional administrations of Cas protein mRNA and/or guide RNA or crRNA might be useful to achieve the most efficient levels of genome modification.

In one embodiment, the systems and methods herein may be used for cleaving a target nucleic acid. The method may comprise modifying a target nucleic acid using a nucleic acid-targeting complex that binds to the target nucleic acid and effect cleavage of said target nucleic acid. In an embodiment, the systems or compositions herein, when introduced into a cell, may create a break (e.g., a single or a double strand break) in the nucleic acid sequence. For example, the systems and methods can be used to cleave a disease nucleic acid in a cell. For example, an exogenous nucleic acid template comprising a sequence to be integrated flanked by an upstream sequence and a downstream sequence may be introduced into a cell. The upstream and downstream sequences share sequence similarity with either side of the site of integration in the nucleic acid. Where desired, a donor nucleic acid can be mRNA. The exogenous nucleic acid template comprises a sequence to be integrated (e.g., a mutated nucleic acid). The sequence for integration may be a sequence endogenous or exogenous to the cell. Thus, the sequence for integration may be operably linked to an appropriate control sequence or sequences. Alternatively, the sequence to be integrated may provide a regulatory function. The upstream and downstream sequences in the exogenous nucleic acid. A template is selected to promote recombination between the nucleic acid sequence of interest and the donor nucleic acid. The upstream sequence may be a nucleic acid sequence that shares sequence similarity with the nucleic acid sequence upstream of the targeted site for integration. Similarly, the downstream sequence may be a nucleic acid sequence that shares sequence similarity with the nucleic acid sequence downstream of the targeted site of integration. The upstream and downstream sequences in the exogenous nucleic acid template can have 75%, 80%, 85%, 90%, 95%, or 100% sequence identity with the targeted nucleic acid sequence. Preferably, the upstream and downstream sequences in the exogenous nucleic acid template have about 95%, 96%, 97%, 98%, 99%, or 100% sequence identity with the targeted sequence. In some cases, the upstream and downstream sequences in the exogenous nucleic acid template have about 99% or 100% sequence identity with the targeted nucleic acid sequence. An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000 bp. In some methods, the exogenous nucleic acid template may further comprise a marker. Such a marker may make it easy to screen for targeted integrations. Examples of suitable markers include restriction sites, fluorescent proteins, or selectable markers. The exogenous nucleic acid template of the present disclosure can be constructed using recombinant techniques (see, for example, Sambrook et al., 2001 and Ausubel et al., 1996). In a method for modifying a target nucleic acid by integrating an exogenous nucleic acid template, a break (e.g., double or single stranded break in double or single stranded nucleic acid) is introduced into the nucleic acid sequence by the nucleic acid-targeting complex, the break is repaired via homologous recombination with an exogenous nucleic acid template such that the template is integrated into the nucleic acid target. The presence of a double-stranded break facilitates integration of the template. In other embodiments, this invention provides a method of modifying expression of a nucleic acid in a eukaryotic cell. The method comprises increasing or decreasing expression of a target polynucleotide by using a nucleic acid-targeting complex that binds to the DNA or RNA (e.g., mRNA or pre-mRNA). In some methods, a target nucleic acid can be inactivated to affect the modification of the expression in a cell. For example, upon the binding of a nucleic acid-targeting complex to a target sequence in a cell, the target nucleic acid is inactivated such that the sequence is not translated, the coded protein is not produced, or the sequence does not function as the wild-type sequence does. For example, a protein or microRNA coding sequence may be inactivated such that the protein or microRNA or pre-microRNA transcript is not produced. The target nucleic acid of a nucleic acid-targeting complex can be any nucleic acid endogenous or exogenous to the eukaryotic cell. For example, the target nucleic acid can be a nucleic acid residing in the nucleus of the eukaryotic cell. The target nucleic acid can be a sequence (e.g., mRNA or pre-mRNA) coding a gene product (e.g., a protein) or a non-coding sequence (e.g., ncRNA, lncRNA, tRNA, or rRNA). Examples of target nucleic acid include a sequence associated with a signaling biochemical pathway, e.g., a signaling biochemical pathway-associated nucleic acid. Examples of target nucleic acid include a disease associated nucleic acid. A “disease-associated” nucleic acid refers to any nucleic acid which is yielding translation products at an abnormal level or in an abnormal form in cells derived from a disease-affected tissues compared with tissues or cells of a non-disease control. It may be a nucleic acid transcribed from a gene that becomes expressed at an abnormally high level; it may be a RNA transcribed from a gene that becomes expressed at an abnormally low level, where the altered expression correlates with the occurrence and/or progression of the disease. A disease-associated nucleic acid also refers to a nucleic acid transcribed from a gene possessing mutation(s) or genetic variation that is directly responsible or is in linkage disequilibrium with a gene(s) that is responsible for the etiology of a disease. The translated products may be known or unknown, and may be at a normal or abnormal level. The target nucleic acid of a nucleic acid-targeting complex can be any nucleic acid endogenous or exogenous to the eukaryotic cell. For example, the target nucleic acid can be a nucleic acid residing in the nucleus of the eukaryotic cell. The target nucleic acid can be a sequence (e.g., mRNA or pre-mRNA) coding a gene product (e.g., a protein) or a non-coding sequence (e.g., ncRNA, lncRNA, tRNA, or rRNA).

In some embodiments, the systems and methods may comprise allowing a nucleic acid-targeting complex to bind to the target nucleic acid to effect cleavage of said target nucleic acid thereby modifying the target nucleic acid, wherein the nucleic acid-targeting complex comprises a nucleic acid-targeting effector (Cas9) protein complexed with a guide RNA or crRNA hybridized to a target sequence within said target nucleic acid. In one aspect, the invention provides a method of modifying expression of nucleic acid in a eukaryotic cell. In some embodiments, the method comprises allowing a nucleic acid-targeting complex to bind to the nucleic acid such that said binding results in increased or decreased expression of said nucleic acid; wherein the nucleic acid-targeting complex comprises a nucleic acid-targeting effector (Cas9) protein complexed with a guide RNA. Methods of modifying a target nucleic acid can be in a eukaryotic cell, which may be in vivo, ex vivo or in vitro. In some embodiments, the method comprises sampling a cell or population of cells from a human or non-human animal, and modifying the cell or cells. Culturing may occur at any stage ex vivo. The cell or cells may even be re-introduced into the non-human animal or plant. For re-introduced cells it is particularly preferred that the cells are stem cells.

The use of two different aptamers (each associated with a distinct nucleic acid-targeting guide RNAs) allows an activator-adaptor protein fusion and a repressor-adaptor protein fusion to be used, with different nucleic acid-targeting guide RNAs or crRNAs, to activate expression of RNA, whilst repressing another. They, along with their different guide RNAs or crRNAs can be administered together, or substantially together, in a multiplexed approach. A large number of such modified RNA-targeting guide RNAs or crRNAs can be used all at the same time, for example 10 or 20 or 30 and so forth, whilst only one (or at least a minimal number) of effector protein (Cas9) molecules need to be delivered, as a comparatively small number of effector protein molecules can be used with a large number of modified guides. The adaptor protein may be associated (preferably linked or fused to) one or more activators or one or more repressors. For example, the adaptor protein may be associated with a first activator and a second activator. The first and second activators may be the same, but they are preferably different activators. Three or more or even four or more activators (or repressors) may be used, but package size may limit the number being higher than 5 different functional domains. Linkers are preferably used, over a direct fusion to the adaptor protein, where two or more functional domains are associated with the adaptor protein. Suitable linkers might include the GlySer linker.

It is also envisaged that the Cas-guide RNA complex as a whole may be associated with two or more functional domains. For example, there may be two or more functional domains associated with the Cas protein, or there may be two or more functional domains associated with the guide RNA or crRNA (via one or more adaptor proteins), or there may be one or more functional domains associated with the Cas protein and one or more functional domains associated with the guide RNA or crRNA (via one or more adaptor proteins).

The fusion between the adaptor protein and the activator or repressor may include a linker. For example, GlySer linkers GGGS can be used. They can be used in repeats of 3 ((GGGGS)₃ or 6, 9 or even 12 or more, to provide suitable lengths, as required. Linkers can be used between the guide RNAs and the functional domain (activator or repressor), or between the nucleic acid-targeting effector protein and the functional domain (activator or repressor). The linkers the user to engineer appropriate amounts of “mechanical flexibility”.

Cas protein or mRNA therefor (or more generally a nucleic acid molecule therefor) and guide RNA or crRNA might also be delivered separately e.g., the former 1-12 hours (preferably around 2-6 hours) prior to the administration of guide RNA or crRNA, or together. A second booster dose of guide RNA or crRNA can be administered 1-12 hours (preferably around 2-6 hours) after the initial administration.

The Cas protein is sometimes referred to herein as a CRISPR Enzyme. It will be appreciated that the effector protein is based on or derived from an enzyme, so the term ‘effector protein’ certainly includes ‘enzyme’ in some embodiments. However, it will also be appreciated that the effector protein may, as required in some embodiments, have DNA or RNA binding, but not necessarily cutting or nicking, activity, including a dead-Cas protein function.

Cellular targets include Hemopoietic Stem/Progenitor Cells (CD34+); Human T cells; and Eye (retinal cells)—for example photoreceptor precursor cells.

The systems may comprise templates. Delivery of templates may be via the cotemporaneous or separate from delivery of any or all the Cas protein or guide or crRNA and via the same delivery mechanism or different.

In certain embodiments, the methods as described herein may comprise providing a Cas transgenic cell in which one or more nucleic acids encoding one or more guide RNAs are provided or introduced operably connected in the cell with a regulatory element comprising a promoter of one or more gene of interest. As used herein, the term “Cas transgenic cell” refers to a cell, such as a eukaryotic cell, in which a Cas gene has been genomically integrated. The nature, type, or origin of the cell are not particularly limiting according to the present invention. Also the way how the Cas transgene is introduced in the cell is may vary and can be any method as is known in the art. In certain embodiments, the Cas transgenic cell is obtained by introducing the Cas transgene in an isolated cell. In certain other embodiments, the Cas transgenic cell is obtained by isolating cells from a Cas transgenic organism. By means of example, and without limitation, the Cas transgenic cell as referred to herein may be derived from a Cas transgenic eukaryote, such as a Cas knock-in eukaryote. Reference is made to WO 2014/093622 (PCT/US13/74667), incorporated herein by reference. Methods of US Patent Publication Nos. 20120017290 and 20110265198 assigned to Sangamo BioSciences, Inc. directed to targeting the Rosa locus may be modified to utilize the CRISPR Cas system. Methods of US Patent Publication No. 20130236946 assigned to Cellectis directed to targeting the Rosa locus may also be modified to utilize the CRISPR Cas system. By means of further example reference is made to Platt et. al. (Cell; 159(2):440-455 (2014)), describing a Cas9 knock-in mouse, which is incorporated herein by reference. The Cas transgene can further comprise a Lox-Stop-polyA-Lox(LSL) cassette thereby rendering Cas expression inducible by Cre recombinase. Alternatively, the Cas transgenic cell may be obtained by introducing the Cas transgene in an isolated cell. Delivery systems for transgenes are well known in the art. By means of example, the Cas transgene may be delivered in for instance eukaryotic cell by means of vector (e.g., AAV, adenovirus, lentivirus) and/or particle and/or particle delivery, as also described herein elsewhere.

It will be understood by the skilled person that the cell, such as the Cas transgenic cell, as referred to herein may comprise further genomic alterations besides having an integrated Cas gene or the mutations arising from the sequence specific action of Cas when complexed with RNA capable of guiding Cas to a target locus, such as for instance one or more oncogenic mutations, as for instance and without limitation described in Platt et al. (2014), Chen et al., (2014) or Kumar et al. (2009).

In some embodiments, the Cas sequence is fused to one or more nuclear localization sequences (NLSs), such as about or more than about 1, 2, 3, 4, 5, 6, 7, 8, 9, 10, or more NLSs. In some embodiments, the Cas comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g. zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In a preferred embodiment, the Cas protein comprises at most 6 NLSs. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Non-limiting examples of NLSs include an NLS sequence derived from: the NLS of the SV40 virus large T-antigen, having the amino acid sequence PKKKRKV (SEQ ID NO: 1); the NLS from nucleoplasmin (e.g. the nucleoplasmin bipartite NLS with the sequence KRPAATKKAGQAKKKK (SEQ ID NO: 2); the c-myc NLS having the amino acid sequence PAAKRVKLD (SEQ ID NO: 3) or RQRRNELKRSP (SEQ ID NO: 4); the hRNPA1 M9 NLS having the sequence NQSSNFGPMKGGNFGGRSSGPYGGGGQYFAKPRNQGGY (SEQ ID NO: 5); the sequence RMRIZFKNKGKDTAELRRRRVEVSVELRKAKKDEQILKRRNV (SEQ ID NO: 6) of the IBB domain from importin-alpha; the sequences VSRKRPRP (SEQ ID NO: 7) and PPKKARED (SEQ ID NO: 8) of the myoma T protein; the sequence PQPKKKPL (SEQ ID NO: 9) of human p53; the sequence SALIKKKKKMAP (SEQ ID NO: 10) of mouse c-abl IV; the sequences DRLRR (SEQ ID NO: 11) and PKQKKRK (SEQ ID NO: 12) of the influenza virus NS1; the sequence RKLKKKIKKL (SEQ ID NO: 13) of the Hepatitis virus delta antigen; the sequence REKKKFLKRR (SEQ ID NO: 14) of the mouse Mx1 protein; the sequence KRKGDEVDGVDEVAKKKSKK (SEQ ID NO: 15) of the human poly(ADP-ribose) polymerase; and the sequence RKCLQAGMNLEARKTKK (SEQ ID NO: 16) of the steroid hormone receptors (human) glucocorticoid. In general, the one or more NLSs are of sufficient strength to drive accumulation of the Cas in a detectable amount in the nucleus of a eukaryotic cell. In general, strength of nuclear localization activity may derive from the number of NLSs in the Cas, the particular NLS(s) used, or a combination of these factors. Detection of accumulation in the nucleus may be performed by any suitable technique. For example, a detectable marker may be fused to the Cas, such that location within a cell may be visualized, such as in combination with a means for detecting the location of the nucleus (e.g. a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of CRISPR complex formation (e.g. assay for DNA cleavage or mutation at the target sequence, or assay for altered gene expression activity affected by CRISPR complex formation and/or Cas enzyme activity), as compared to a control no exposed to the Cas or complex, or exposed to a Cas lacking the one or more NLSs. In certain embodiments of the herein described Cas9 effector protein complexes and systems the codon optimized Cas9 effector proteins comprise an NLS attached to the C-terminal of the protein. In certain embodiments, other localization tags may be fused to the Cas protein, such as without limitation for localizing the Cas to particular sites in a cell, such as organelles, such mitochondria, plastids, chloroplast, vesicles, golgi, (nuclear or cellular) membranes, ribosomes, nucleolus, ER, cytoskeleton, vacuoles, centrosome, nucleosome, granules, centrioles, etc.

The guide RNA(s), e.g., sgRNA(s) or crRNA(s) encoding sequences and/or Cas encoding sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the β-actin promoter, the phosphoglycerol kinase (PGK) promoter, and the EF1α promoter. An advantageous promoter is the promoter is U6.

In some embodiments, a Cas protein (e.g., Cas9 protein) may form a component of an inducible system. The inducible nature of the system would allow for spatiotemporal control of gene editing or gene expression using a form of energy. The form of energy may include but is not limited to electromagnetic radiation, sound energy, chemical energy and thermal energy. Examples of inducible system include tetracycline inducible promoters (Tet-On or Tet-Off), small molecule two-hybrid transcription activations systems (FKBP, ABA, etc.), or light inducible systems (Phytochrome, LOV domains, or cryptochrome). In one embodiment, the CRISPR effector protein may be a part of a Light Inducible Transcriptional Effector (LITE) to direct changes in transcriptional activity in a sequence-specific manner. The components of a light may include a CRISPR effector protein, a light-responsive cytochrome heterodimer (e.g. from Arabidopsis thaliana), and a transcriptional activation/repression domain. Further examples of inducible DNA binding proteins and methods for their use are provided in U.S. 61/736,465 and U.S. 61/721,283, and WO 2014018423 A2 which is hereby incorporated by reference in its entirety.

In one aspect, the present disclosure provides a mutated Cas (e.g., Cas9) as described herein elsewhere, having one or more mutations resulting in reduced off-target effects, e.g., improved CRISPR enzymes for use in effecting modifications to target loci but which reduce or eliminate activity towards off-targets, such as when complexed to guide RNAs, as well as improved CRISPR enzymes for increasing the activity of CRISPR enzymes, such as when complexed with guide RNAs. It is to be understood that mutated enzymes as described herein below may be used in any of the methods according to the present disclosure as described herein elsewhere. Any of the methods, products, compositions and uses as described herein elsewhere are equally applicable with the mutated CRISPR enzymes as further detailed below.

Slaymaker et al. recently described a method for the generation of Cas9 orthologues with enhanced specificity (Slaymaker et al. 2015 “Rationally engineered Cas9 nucleases with improved specificity”). This strategy can be used to enhance the specificity of the Cas protein. Primary residues for mutagenesis are preferably all positive charges residues within the RuvC and/or HNH domain. Additional residues are positive charged residues that are conserved between different orthologues.

In an aspect, the present disclosure also provides methods and mutations for modulating Cas binding activity and/or binding specificity. In certain embodiments Cas proteins lacking nuclease activity are used. In certain embodiments, modified guide RNAs are employed that promote binding but not nuclease activity of a Cas nuclease. In such embodiments, on-target binding can be increased or decreased. Also, in such embodiments off-target binding can be increased or decreased. Moreover, there can be increased or decreased specificity as to on-target binding vs. off-target binding.

The methods and mutations which can be employed in various combinations to increase or decrease activity and/or specificity of on-target vs. off-target activity, or increase or decrease binding and/or specificity of on-target vs. off-target binding, can be used to compensate or enhance mutations or modifications made to promote other effects. Such mutations or modifications made to promote other effects in include mutations or modification to the Cas and or mutation or modification made to a guide RNA. The methods and mutations of the present disclosure are used to modulate Cas nuclease activity and/or binding with chemically modified guide RNAs.

In an aspect, the present disclosure provides methods and mutations for modulating binding and/or binding specificity of Cas proteins according to the present disclosure as defined herein comprising functional domains such as nucleases, transcriptional activators, transcriptional repressors, and the like. For example, a Cas protein can be made nuclease-null, or having altered or reduced nuclease activity by introducing mutations such as for instance Cas mutations described herein elsewhere. Nuclease deficient Cas proteins are useful for RNA-guided target sequence dependent delivery of functional domains. The present disclosure provides methods and mutations for modulating binding of Cas proteins. In one embodiment, the functional domain comprises VP64, providing an RNA-guided transcription factor. In another embodiment, the functional domain comprises Fok I, providing an RNA-guided nuclease activity. Mention is made of U.S. Pat. Pub. 2014/0356959, U.S. Pat. Pub. 2014/0342456, U.S. Pat. Pub. 2015/0031132, and Mali, P. et al., 2013, Science 339(6121):823-6, doi: 10.1126/science.1232033, published online 3 Jan. 2013 and through the teachings herein the present disclosure comprehends methods and materials of these documents applied in conjunction with the teachings herein. In certain embodiments, on-target binding is increased. In certain embodiments, off-target binding is decreased. In certain embodiments, on-target binding is decreased. In certain embodiments, off-target binding is increased. Accordingly, the present disclosure also provides for increasing or decreasing specificity of on-target binding vs. off-target binding of functionalized Cas binding proteins.

The use of Cas as an RNA-guided binding protein is not limited to nuclease-null Cas. Cas enzymes comprising nuclease activity can also function as RNA-guided binding proteins when used with certain guide RNAs. For example, short guide RNAs and guide RNAs comprising nucleotides mismatched to the target can promote RNA directed Cas binding to a target sequence with little or no target cleavage. (See, e.g., Dahlman, 2015, Nat Biotechnol. 33(11):1159-1161, doi: 10.1038/nbt.3390, published online 5 Oct. 2015). In an aspect, the present disclosure provides methods and mutations for modulating binding of Cas proteins that comprise nuclease activity. In certain embodiments, on-target binding is increased. In certain embodiments, off-target binding is decreased. In certain embodiments, on-target binding is decreased. In certain embodiments, off-target binding is increased. In certain embodiments, there is increased or decreased specificity of on-target binding vs. off-target binding. In certain embodiments, nuclease activity of guide RNA-Cas enzyme is also modulated.

RNA-RNA duplex formation is important for cleavage activity and specificity throughout the target region, not only the seed region sequence closest to the PAM. Thus, truncated guide RNAs show reduced cleavage activity and specificity. In an aspect, the present disclosure provides method and mutations for increasing activity and specificity of cleavage using altered guide RNAs.

In certain embodiments, the catalytic activity of the Cas protein (e.g., Cas9) of the present disclosure is altered or modified. It is to be understood that mutated Cas has an altered or modified catalytic activity if the catalytic activity is different than the catalytic activity of the corresponding wild type Cas protein (e.g., unmutated Cas protein). Catalytic activity can be determined by means known in the art. By means of example, and without limitation, catalytic activity can be determined in vitro or in vivo by determination of indel percentage (for instance after a given time, or at a given dose). In certain embodiments, catalytic activity is increased. In certain embodiments, catalytic activity is increased by at least 5%, preferably at least 10%, more preferably at least 20%, such as at least 30%, at least 40%, at least 50%, at least 60%, at least 70%, at least 80%, at least 90%, or at least 100%. In certain embodiments, catalytic activity is decreased. In certain embodiments, catalytic activity is decreased by at least 5%, preferably at least 10%, more preferably at least 20%, such as at least 30%, at least 40%, at least 50%, at least 60%, at least 70%, at least 80%, at least 90%, or (substantially) 100%. The one or more mutations herein may inactivate the catalytic activity, which may substantially all catalytic activity, below detectable levels, or no measurable catalytic activity.

One or more characteristics of the engineered Cas protein may be different from a corresponding wiled type Cas protein. Examples of such characteristics include catalytic activity, gRNA binding, specificity of the Cas protein (e.g., specificity of editing a defined target), stability of the Cas protein, off-target binding, target binding, protease activity, nickase activity, PAM recognition. In some examples, a engineered Cas protein may comprise one or more mutations of the corresponding wild type Cas protein. In some embodiments, the catalytic activity of the engineered Cas protein is increased as compared to a corresponding wildtype Cas protein. In some embodiments, the catalytic activity of the engineered Cas protein is decreased as compared to a corresponding wildtype Cas protein. In some embodiments, the gRNA binding of the engineered Cas protein is increased as compared to a corresponding wildtype Cas protein. In some embodiments, the gRNA binding of the engineered Cas protein is decreased as compared to a corresponding wildtype Cas protein. In some embodiments, the specificity of the Cas protein is increased as compared to a corresponding wildtype Cas protein. In some embodiments, the specificity of the Cas protein is decreased as compared to a corresponding wildtype Cas protein. In some embodiments, the stability of the Cas protein is increased as compared to a corresponding wildtype Cas protein. In some embodiments, the stability of the Cas protein is decreased as compared to a corresponding wildtype Cas protein. In some embodiments, the engineered Cas protein further comprises one or more mutations which inactivate catalytic activity. In some embodiments, the off-target binding of the Cas protein is increased as compared to a corresponding wildtype Cas protein. In some embodiments, the off-target binding of the Cas protein is decreased as compared to a corresponding wildtype Cas protein. In some embodiments, the target binding of the Cas protein is increased as compared to a corresponding wildtype Cas protein. In some embodiments, the target binding of the Cas protein is decreased as compared to a corresponding wildtype Cas protein. In some embodiments, the engineered Cas protein has a higher protease activity or polynucleotide-binding capability compared with a corresponding wildtype Cas protein. In some embodiments, the PAM recognition is altered as compared to a corresponding wildtype Cas protein.

In certain embodiments, the gRNA (crRNA) binding of the Cas protein of the present disclosure is altered or modified. It is to be understood that mutated Cas has an altered or modified gRNA binding if the gRNA binding is different than the gRNA binding of the corresponding wild type Cas (i.e. unmutated Cas). gRNA binding can be determined by means known in the art. By means of example, and without limitation, gRNA binding can be determined by calculating binding strength or affinity (such as based on equilibrium constants, Ka, Kd, etc.). In certain embodiments, gRNA binding is increased. In certain embodiments, gRNA binding is increased by at least 5%, preferably at least 10%, more preferably at least 20%, such as at least 30%, at least 40%, at least 50%, at least 60%, at least 70%, at least 80%, at least 90%, or at least 100%. In certain embodiments, gRNA binding is decreased. In certain embodiments, gRNA binding is decreased by at least 5%, preferably at least 10%, more preferably at least 20%, such as at least 30%, at least 40%, at least 50%, at least 60%, at least 70%, at least 80%, at least 90%, or (substantially) 100%.

In certain embodiments, the specificity of the Cas protein of the present disclosure is altered or modified. It is to be understood that mutated Cas has an altered or modified specificity if the specificity is different than the specificity of the corresponding wild type Cas (i.e. unmutated Cas). Specificity can be determined by means known in the art. By means of example, and without limitation, specificity can be determined by comparison of on-target activity and off-target activity. In certain embodiments, specificity is increased. In certain embodiments, specificity is increased by at least 5%, preferably at least 10%, more preferably at least 20%, such as at least 30%, at least 40%, at least 50%, at least 60%, at least 70%, at least 80%, at least 90%, or at least 100%. In certain embodiments, specificity is decreased. In certain embodiments, specificity is decreased by at least 5%, preferably at least 10%, more preferably at least 20%, such as at least 30%, at least 40%, at least 50%, at least 60%, at least 70%, at least 80%, at least 90%, or (substantially) 100%.

In certain embodiments, the stability of the Cas protein of the present disclosure is altered or modified. It is to be understood that mutated Cas has an altered or modified stability if the stability is different than the stability of the corresponding wild type Cas (i.e. unmutated Cas). Stability can be determined by means known in the art. By means of example, and without limitation, stability can be determined by determining the half-life of the Cas protein. In certain embodiments, stability is increased. In certain embodiments, stability is increased by at least 5%, preferably at least 10%, more preferably at least 20%, such as at least 30%, at least 40%, at least 50%, at least 60%, at least 70%, at least 80%, at least 90%, or at least 100%. In certain embodiments, stability is decreased. In certain embodiments, stability is decreased by at least 5%, preferably at least 10%, more preferably at least 20%, such as at least 30%, at least 40%, at least 50%, at least 60%, at least 70%, at least 80%, at least 90%, or (substantially) 100%.

In certain embodiments, the target binding of the Cas protein of the present disclosure is altered or modified. It is to be understood that mutated Cas has an altered or modified target binding if the target binding is different than the target binding of the corresponding wild type Cas (i.e. unmutated Cas). target binding can be determined by means known in the art. By means of example, and without limitation, target binding can be determined by calculating binding strength or affinity (such as based on equilibrium constants, Ka, Kd, etc.). In certain embodiments, target bindings increased. In certain embodiments, target binding is increased by at least 5%, preferably at least 10%, more preferably at least 20%, such as at least 30%, at least 40%, at least 50%, at least 60%, at least 70%, at least 80%, at least 90%, or at least 100%. In certain embodiments, target binding is decreased. In certain embodiments, target binding is decreased by at least 5%, preferably at least 10%, more preferably at least 20%, such as at least 30%, at least 40%, at least 50%, at least 60%, at least 70%, at least 80%, at least 90%, or (substantially) 100%.

In certain embodiments, the off-target binding of the Cas protein of the present disclosure is altered or modified. It is to be understood that mutated Cas has an altered or modified off-target binding if the off-target binding is different than the off-target binding of the corresponding wild type Cas (i.e. unmutated Cas). Off-target binding can be determined by means known in the art. By means of example, and without limitation, off-target binding can be determined by calculating binding strength or affinity (such as based on equilibrium constants, Ka, Kd, etc.). In certain embodiments, off-target bindings increased. In certain embodiments, off-target binding is increased by at least 5%, preferably at least 10%, more preferably at least 20%, such as at least 30%, at least 40%, at least 50%, at least 60%, at least 70%, at least 80%, at least 90%, or at least 100%. In certain embodiments, off-target binding is decreased. In certain embodiments, off-target binding is decreased by at least 5%, preferably at least 10%, more preferably at least 20%, such as at least 30%, at least 40%, at least 50%, at least 60%, at least 70%, at least 80%, at least 90%, or (substantially) 100%.

In certain embodiments, the PAM recognition or specificity of the Cas protein of the present disclosure is altered or modified. It is to be understood that mutated Cas has an altered or modified PAM recognition or specificity if the PAM recognition or specificity is different than the PAM recognition or specificity of the corresponding wild type Cas (i.e. unmutated Cas). PAM recognition or specificity can be determined by means known in the art. By means of example, and without limitation, PAM recognition or specificity can be determined by PAM screens. In certain embodiments, at least one different PAM is recognized by the Cas. In certain embodiments, at least one PAM is recognized by the mutated Cas which is not recognized by the corresponding wild type Cas. In certain embodiments, at least one PAM is recognized by the mutated Cas which is not recognized by the corresponding wild type Cas, in addition to the wild type PAM. In certain embodiments, at least one PAM is recognized by the mutated Cas which is not recognized by the corresponding wild type Cas, and the wild type PAM is not anymore recognized. In certain embodiments, the PAM recognized by the mutated Cas is longer than the PAM recognized by the wild type Cas, such as 1, 2, or 3 nucleotides longer. In certain embodiments, the PAM recognized by the mutated Cas is shorter than the PAM recognized by the wild type Cas, such as 1, 2, or 3 nucleotides shorter. In some examples, the Cas9-t may recognize or interfere with a PAM comprising NGCH.

In some embodiments, the present disclosure provides a non-naturally occurring or engineered composition comprising i) a mutated Cas protein, and ii) a crRNA, wherein the crRNA comprises a) a guide sequence that is capable of hybridizing to a target RNA sequence, and b) a direct repeat sequence, whereby there is formed a CRISPR complex comprising the Cas protein complexed with the guide sequence that is hybridized to the target RNA sequence. The complex can be formed in vitro or ex vivo and introduced into a cell or contacted with RNA; or can be formed in vivo.

In some embodiments, such as for Cas, a non-naturally occurring or engineered composition of the present disclosure may comprise an accessory protein that enhances the Cas protein activity. In such embodiments, the Cas protein and the accessory protein may be from the same source or from a different source. In some embodiments, a non-naturally occurring or engineered composition of the present disclosure comprises an accessory protein that represses Cas protein activity. In some embodiments, a non-naturally occurring or engineered composition of the present disclosure comprises two or more crRNAs. In some embodiments, a non-naturally occurring or engineered composition of the present disclosure comprises a guide sequence that hybridizes to a target RNA sequence in a prokaryotic cell. In some embodiments, a non-naturally occurring or engineered composition of the present disclosure comprises a guide sequence that hybridizes to a target RNA sequence in a eukaryotic cell. In some embodiment, the Cas protein comprises one or more nuclear localization signals (NLSs).

In some embodiments of the non-naturally occurring or engineered composition of the present disclosure, the Cas protein is associated with one or more functional domains. The association can be by direct linkage of the effector protein to the functional domain, or by association with the crRNA. In a non-limiting example, the crRNA comprises an added or inserted sequence that can be associated with a functional domain of interest, including, for example, an aptamer or a nucleotide that binds to a nucleic acid binding adapter protein. The functional domain may be a functional heterologous domain.

In certain non-limiting embodiments, a non-naturally occurring or engineered composition of the present disclosure comprises a functional domain cleaves the target RNA sequence. In certain non-limiting embodiments, the non-naturally occurring or engineered composition of the present disclosure comprises a functional domain that modifies transcription or translation of the target RNA sequence.

In some embodiment of the composition of the present disclosure, the Cas protein is associated with one or more functional domains; and the effector protein contains one or more mutations within a RuvC and/or HNH domain, whereby the complex can deliver an epigenetic modifier or a transcriptional or translational activation or repression signal. The complex can be formed in vitro or ex vivo and introduced into a cell or contacted with RNA; or can be formed in vivo.

In some embodiments of the non-naturally occurring or engineered composition of the present disclosure, the Cas protein and the accessory protein are from the same organism. In some embodiments of the non-naturally occurring or engineered composition of the present disclosure, the Cas protein and the accessory protein are from different organisms.

The present disclosure further provides a vector system. The vector system may comprise one or more polynucleotides. The polynucleotide(s) comprise one or more sequences coding for the components of a CRISPR-Cas system, Cas proteins and guide molecules. The polynucleotides may further comprise templates or coding sequence thereof. In some examples, a vector system may comprise one or more vectors comprising: a first regulatory element operably linked to a nucleotide sequence encoding the Cas protein, and a second regulatory element operably linked to a nucleotide sequence encoding the crRNA.

In certain embodiments, the vector system of the present disclosure further comprises a regulatory element operably linked to a nucleotide sequence of a Type II CRISPR-Cas accessory protein. When appropriate, the nucleotide sequence encoding the Type II CRISPR-Cas protein (and/or optionally the nucleotide sequence encoding the Type II CRISPR-Cas accessory protein) is codon optimized for expression in a eukaryotic cell. In some embodiments of the vector system of the present disclosure, the nucleotide sequences encoding the Cas protein (and optionally) the accessory protein are codon optimized for expression in a eukaryotic cell. In some embodiments, the vector system of the present disclosure comprises in a single vector. In some embodiment of the vector system of the present disclosure, the one or more vectors comprise viral vectors. In some embodiment of the vector system of the present disclosure, the one or more vectors comprise one or more retroviral, lentiviral, adenoviral, adeno-associated or Herpes simplex viral vectors.

In some embodiments the present disclosure provides a method of modifying expression of a target gene of interest, the method comprising contacting a target RNA with one or more non-naturally occurring or engineered compositions comprising i) a mutated Cas protein according to the present disclosure as described herein, and ii) a crRNA, wherein the crRNA comprises a) a guide sequence that hybridizes to a target RNA sequence in a cell, and b) a direct repeat sequence, wherein the Cas protein forms a complex with the crRNA, wherein the guide sequence directs sequence-specific binding to the target RNA sequence in a cell, whereby there is formed a CRIB R. complex comprising the Cas protein complexed with the guide sequence that is hybridized to the target RNA sequence, whereby expression of the target locus of interest is modified. The complex can be formed in vitro or ex vivo and introduced into a cell or contacted with RNA; or can be formed in vivo.

In some embodiments of the method of modifying expression of a target gene of interest, the target gene is in a prokaryotic cell. In some embodiments of the method of modifying expression of a target gene of interest, the target gene is in a eukaryotic cell. In some embodiments the present disclosure provides a cell comprising a modified target of interest, wherein the target of interest has been modified according to any of the method disclosed herein. In some embodiments, the cell is a prokaryotic cell. In some embodiments, the cell is a eukaryotic cell.

In some embodiments, modification of the target of interest in a cell results in: a cell comprising altered expression of at least one gene product; a cell comprising altered expression of at least one gene product, wherein the expression of the at least one gene product is increased; or a cell comprising altered expression of at least one gene product, wherein the expression of the at least one gene product is decreased.

In some embodiments, the cell is a mammalian cell or a human cell.

In some embodiments the present disclosure provides a cell line of or comprising a cell disclosed herein or a cell modified by any of the methods disclosed herein, or progeny thereof.

In some embodiments the present disclosure provides a multicellular organism comprising one or more cells disclosed herein or one or more cells modified according to any of the methods disclosed herein.

In some embodiments the present disclosure provides a plant or animal model comprising one or more cells disclosed herein or one or more cells modified according to any of the methods disclosed herein.

In some embodiments the present disclosure provides a gene product from a cell or the cell line or the organism or the plant or animal model disclosed herein.

In some embodiment, the amount of gene product expressed is greater than or less than the amount of gene product from a cell that does not have altered expression.

In some embodiments the present disclosure provides a method of identifying the requirements of a suitable guide sequence for the Cas protein of the present disclosure, said method comprising: (a) selecting a set of essential genes within an organism, (b) designing a library of targeting guide sequences capable of hybridizing to regions the coding regions of these genes as well as 5′ and 3′ UTRs of these genes, (c) generating randomized guide sequences that do not hybridize to any region within the genome of said organism as control guides, (d) preparing a plasmid comprising the nucleic acid-targeting protein and a first resistance gene and a guide plasmid library comprising said library of targeting guides and said control guides and a second resistance gene, (e) co-introducing said plasmids into a host cell, (f) introducing said host cells on a selective medium for said first and second resistance genes, (g) sequencing essential genes of growing host cells, (h) determining significance of depletion of cells transformed with targeting guides by comparing depletion of cells with control guides; and, (i) determining based on the depleted guide sequences the requirements of a suitable guide sequence.

In one aspect, determining the PAM sequence for suitable guide sequence of the nucleic acid-targeting protein is by comparison of sequences targeted by guides in depleted cells. In one aspect of such method, the method further comprises comparing the guide abundance for the different conditions in different replicate experiments. In one aspect of such method, the control guides are selected in that they are determined to show limited deviation in guide depletion in replicate experiments. In one aspect of such method, the significance of depletion is determined as (a) a depletion which is more than the most depleted control guide; or (b) a depletion which is more than the average depletion plus two times the standard deviation for the control guides. In one aspect of such method, the host cell is a bacterial host cell. In one aspect of such method, the step of co-introducing the plasmids is by electroporation and the host cell is an electro-competent host cell.

In some embodiments the present disclosure provides a method of modifying sequences associated with or at a target locus of interest, the method comprising delivering to said locus a non-naturally occurring or engineered composition comprising a Cas protein and one or more nucleic acid components, wherein the effector protein forms a complex with the one or more nucleic acid components and upon binding of the said complex to the locus of interest the effector protein induces the modification of the sequences associated with or at the target locus of interest. In a preferred embodiment, the modification is the introduction of a strand break. In a preferred embodiment, the sequences associated with or at the target locus of interest comprises RNA or consists of RNA.

In some embodiments the present disclosure provides a method of modifying sequences associated with or at a target locus of interest, the method comprising delivering to said locus a non-naturally occurring or engineered composition comprising a Cas protein, optionally a small accessory protein, and one or more nucleic acid components, wherein the effector protein forms a complex with the one or more nucleic acid components and upon binding of the said complex to the locus of interest the effector protein induces the modification of the sequences associated with or at the target locus of interest. In a preferred embodiment, the modification is the introduction of a strand break. In a preferred embodiment, the sequences associated with or at the target locus of interest comprises RNA or consists of RNA.

In some embodiments the present disclosure provides a method of modifying sequences associated with or at a target locus of interest, the method comprising delivering to said sequences associated with or at the locus a non-naturally occurring or engineered composition comprising a Cas loci effector protein and one or more nucleic acid components, wherein the Cas protein forms a complex with the one or more nucleic acid components and upon binding of the said complex to the locus of interest the effector protein induces the modification of sequences associated with or at the target locus of interest. In a preferred embodiment, the modification is the introduction of a strand break. In a preferred embodiment the Cas protein forms a complex with one nucleic acid component; advantageously an engineered or non-naturally occurring nucleic acid component. The induction of modification of sequences associated with or at the target locus of interest can be Cas protein-nucleic acid guided. In a preferred embodiment the one nucleic acid component is a CRISPR RNA (crRNA). In a preferred embodiment the one nucleic acid component is a mature crRNA or guide RNA, wherein the mature crRNA or guide RNA comprises a spacer sequence (or guide sequence) and a direct repeat (DR) sequence or derivatives thereof. In a preferred embodiment the spacer sequence or the derivative thereof comprises a seed sequence, wherein the seed sequence is critical for recognition and/or hybridization to the sequence at the target locus. In a preferred embodiment of the present disclosure the crRNA is a short crRNA that may be associated with a short DR sequence. In another embodiment of the present disclosure the crRNA is a long crRNA that may be associated with a long DR sequence (or dual DR). Aspects of the present disclosure relate to Cas protein complexes having one or more non-naturally occurring or engineered or modified or optimized nucleic acid components. In a preferred embodiment the nucleic acid component comprises RNA. In a preferred embodiment the nucleic acid component of the complex may comprise a guide sequence linked to a direct repeat sequence, wherein the direct repeat sequence comprises one or more stem loops or optimized secondary structures. In preferred embodiments of the present disclosure, the direct repeat may be a short DR or a long DR (dual DR). In a preferred embodiment the direct repeat may be modified to comprise one or more protein-binding RNA aptamers. In a preferred embodiment, one or more aptamers may be included such as part of optimized secondary structure. Such aptamers may be capable of binding a bacteriophage coat protein. In a preferred embodiment, the bacteriophage coat protein is MS2. The present disclosure also provides for the nucleic acid component of the complex being 30 or more, 40 or more or 50 or more nucleotides in length.

In some embodiments the present disclosure provides methods of genome editing or modifying sequences associated with or at a target locus of interest wherein the method comprises introducing a Cas complex into any desired cell type, prokaryotic or eukaryotic cell, whereby the Cas protein complex effectively functions to interfere with RNA in the eukaryotic or prokaryotic cell. In preferred embodiments, the cell is a eukaryotic cell and the RNA is transcribed from a mammalian genome or is present in a mammalian cell. In preferred methods of RNA editing or genome editing in human cells, the Cas proteins may include but are not limited to the specific species of Cas proteins disclosed herein.

In some embodiments the present disclosure also provides a method of modifying a target locus of interest, the method comprising delivering to said locus a non-naturally occurring or engineered composition comprising a Cas protein and one or more nucleic acid components, wherein the Cas protein forms a complex with the one or more nucleic acid components and upon binding of the said complex to the locus of interest the effector protein induces the modification of the target locus of interest. In a preferred embodiment, the modification is the introduction of a strand break.

In such methods the target locus of interest may be comprised within a RNA molecule. In such methods the target locus of interest may be comprised in a RNA molecule in vitro.

In such methods the target locus of interest may be comprised in a RNA molecule within a cell. The cell may be a prokaryotic cell or a eukaryotic cell. The cell may be a mammalian cell. The modification introduced to the cell by the present disclosure may be such that the cell and progeny of the cell are altered for improved production of biologic products such as an antibody, starch, alcohol or other desired cellular output. The modification introduced to the cell by the present disclosure may be such that the cell and progeny of the cell include an alteration that changes the biologic product produced.

The mammalian cell many be a non-human mammal, e.g., primate, bovine, ovine, porcine, canine, rodent, Leporidae such as monkey, cow, sheep, pig, dog, rabbit, rat or mouse cell. The cell may be a non-mammalian eukaryotic cell such as poultry bird (e.g., chicken), vertebrate fish (e.g., salmon) or shellfish (e.g., oyster, claim, lobster, shrimp) cell. The cell may also be a plant cell. The plant cell may be of a monocot or dicot or of a crop or grain plant such as cassava, corn, sorghum, soybean, wheat, oat or rice. The plant cell may also be of an algae, tree or production plant, fruit or vegetable (e.g., trees such as citrus trees, e.g., orange, grapefruit or lemon trees; peach or nectarine trees; apple or pear trees; nut trees such as almond or walnut or pistachio trees; nightshade plants; plants of the genus Brassica; plants of the genus Lectica; plants of the genus Spinalis; plants of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa).

In some embodiments the present disclosure provides a method of modifying a target locus of interest, the method comprising delivering to said locus a non-naturally occurring or engineered composition comprising a Cas protein and one or more nucleic acid components, wherein the effector protein forms a complex with the one or more nucleic acid components and upon binding of the said complex to the locus of interest the effector protein induces the modification of the target locus of interest. In a preferred embodiment, the modification is the introduction of a strand break.

In such methods the target locus of interest may be comprised within an RNA molecule. In a preferred embodiment, the target locus of interest comprises or consists of RNA.

In some embodiments the present disclosure also provides a method of modifying a target locus of interest, the method comprising delivering to said locus a non-naturally occurring or engineered composition comprising a Cas protein and one or more nucleic acid components, wherein the Cas protein forms a complex with the one or more nucleic acid components and upon binding of the said complex to the locus of interest the effector protein induces the modification of the target locus of interest. In a preferred embodiment, the modification is the introduction of a strand break.

In some embodiments, in such methods the target locus of interest may be comprised in a RNA molecule in vitro. Also preferably, in such methods the target locus of interest may be comprised in a RNA molecule within a cell. The cell may be a prokaryotic cell or a eukaryotic cell. The cell may be a mammalian cell. The cell may be a rodent cell. The cell may be a mouse cell.

In any of the described methods the target locus of interest may be a genomic or epigenomic locus of interest. In any of the described methods the complex may be delivered with multiple guides for multiplexed use. In any of the described methods more than one protein(s) may be used.

In further aspects of the present disclosure the nucleic acid components may comprise a CRISPR RNA (crRNA) sequence. As the effector protein is a Cas protein, the nucleic acid components may comprise a CRISPR RNA (crRNA) sequence and generally may not comprise any trans-activating crRNA (tracr RNA) sequence.

In any of the described methods the effector protein and nucleic acid components may be provided via one or more polynucleotide molecules encoding the protein and/or nucleic acid component(s), and wherein the one or more polynucleotide molecules are operably configured to express the protein and/or the nucleic acid component(s). The one or more polynucleotide molecules may comprise one or more regulatory elements operably configured to express the protein and/or the nucleic acid component(s). The one or more polynucleotide molecules may be comprised within one or more vectors. In any of the described methods the target locus of interest may be a genomic, epigenomic, or transcriptomic locus of interest.

The present disclosure also provides a non-naturally occurring or engineered composition which is a composition having the characteristics as discussed herein or defined in any of the herein described methods.

In certain embodiments, the present disclosure thus provides a non-naturally occurring or engineered composition, such as particularly a composition capable of or configured to modify a target locus of interest, said composition comprising a Cas protein and one or more nucleic acid components, wherein the effector protein forms a complex with the one or more nucleic acid components and upon binding of the said complex to the locus of interest the effector protein induces the modification of the target locus of interest.

The present disclosure also provides in a further aspect a non-naturally occurring or engineered composition, such as particularly a composition capable of or configured to modify a target locus of interest, said composition comprising: (a) a guide RNA molecule (or a combination of guide RNA molecules, e.g., a first guide RNA molecule and a second guide RNA molecule) or a nucleic acid encoding the guide RNA molecule (or one or more nucleic acids encoding the combination of guide RNA molecules); (b) a Cas protein. In certain embodiments, the effector protein may be a Cas9 protein.

The present disclosure also provides in a further aspect a non-naturally occurring or engineered composition comprising: (I.) one or more CRISPR-Cas system polynucleotide sequences comprising (a) a guide sequence capable of hybridizing to a target sequence in a polynucleotide locus, (b) a tracr mate (i.e. direct repeat) sequence, and (II.) a second polynucleotide sequence encoding a Cas protein, wherein when transcribed, the guide sequence directs sequence-specific binding of a CRISPR complex to the target sequence, and wherein the CRISPR complex comprises the Cas protein complexed with the guide sequence that is hybridized to the target sequence. In certain embodiments, the effector protein may be a Cas protein.

In certain embodiments, a tracrRNA may not be required. Hence, the present disclosure also provides in certain embodiments a non-naturally occurring or engineered composition comprising: (I.) one or more CRISPR-Cas system polynucleotide sequences comprising (a) a guide sequence capable of hybridizing to a target sequence in a polynucleotide locus, and (b) a direct repeat sequence, and (II.) a second polynucleotide sequence encoding a Cas protein, wherein when transcribed, the guide sequence directs sequence-specific binding of a CRISPR complex to the target sequence, and wherein the CRISPR complex comprises the Cas protein complexed with (1) the guide sequence that is hybridized to the target sequence, and (2) the direct repeat sequence. Preferably, the effector protein may be a Cas protein. Without limitation, the Applicants hypothesize that in such instances, the direct repeat sequence may comprise secondary structure that is sufficient for crRNA loading onto the effector protein. By means of example and not limitation, such secondary structure may comprise, consist essentially of or consist of a stem loop (such as one or more stem loops) within the direct repeat.

The present disclosure also provides a vector system comprising one or more vectors, the one or more vectors comprising one or more polynucleotide molecules encoding components of a non-naturally occurring or engineered composition which is a composition having the characteristics as defined in any of the herein described methods.

The present disclosure also provides a delivery system comprising one or more vectors or one or more polynucleotide molecules, the one or more vectors or polynucleotide molecules comprising one or more polynucleotide molecules encoding components of a non-naturally occurring or engineered composition which is a composition having the characteristics discussed herein or as defined in any of the herein described methods.

The present disclosure also provides a non-naturally occurring or engineered composition, or one or more polynucleotides encoding components of said composition, or vector or delivery systems comprising one or more polynucleotides encoding components of said composition for use in a therapeutic method of treatment. The therapeutic method of treatment may comprise gene or genome editing, or gene therapy.

The present disclosure also provides for methods and compositions wherein one or more amino acid residues of the effector protein may be modified e.g., an engineered or non-naturally-occurring Cas protein of or comprising or consisting or consisting essentially a Tables 1-5 protein. In an embodiment, the modification may comprise mutation of one or more amino acid residues of the effector protein. The one or more mutations may be in one or more catalytically active domains of the effector protein. The effector protein may have reduced or abolished nuclease activity compared with an effector protein lacking said one or more mutations. The effector protein may not direct cleavage of one RNA strand at the target locus of interest. In a preferred embodiment, the one or more mutations may comprise two mutations. In a preferred embodiment the one or more amino acid residues are modified in the Cas protein, e.g., an engineered or non-naturally-occurring Cas protein. The effector protein may comprise one or more heterologous functional domains. The one or more heterologous functional domains may comprise one or more nuclear localization signal (NLS) domains. The one or more heterologous functional domains may comprise at least two or more NLS domains. The one or more NLS domain(s) may be positioned at or near or in proximity to a terminus of the effector protein (e.g., Cas9 protein) and if two or more NLSs, each of the two may be positioned at or near or in proximity to a terminus of the effector protein (e.g., Cas protein). The one or more heterologous functional domains may comprise one or more transcriptional activation domains. In a preferred embodiment the transcriptional activation domain may comprise VP64. The one or more heterologous functional domains may comprise one or more transcriptional repression domains. In a preferred embodiment the transcriptional repression domain comprises a KRAB domain or a SID domain (e.g. SID4X). The one or more heterologous functional domains may comprise one or more nuclease domains. In a preferred embodiment a nuclease domain comprises Fok1.

The present disclosure also provides for the one or more heterologous functional domains to have one or more of the following activities: methylase activity, demethylase activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity and nucleic acid binding activity. At least one or more heterologous functional domains may be at or near the amino-terminus of the effector protein and/or wherein at least one or more heterologous functional domains is at or near the carboxy-terminus of the effector protein. The one or more heterologous functional domains may be fused to the effector protein. The one or more heterologous functional domains may be tethered to the effector protein. The one or more heterologous functional domains may be linked to the effector protein by a linker moiety.

In certain embodiments, the Cas proteins herein may be associated with a locus comprising short CRISPR repeats between 30 and 40 bp long, more typically between 34 and 38 bp long, even more typically between 36 and 37 bp long, e.g., 30, 31, 32, 33, 34, 35, 36, 37, 38, 39, or 40 bp long. In certain embodiments the CRISPR repeats are long or dual repeats between 80 and 350 bp long such as between 80 and 200 bp long, even more typically between 86 and 88 bp long, e.g., 80, 81, 82, 83, 84, 85, 86, 87, 88, 89, or 90 bp long

In certain embodiments, a protospacer adjacent motif (PAM) or PAM-like motif directs binding of the Cas protein complex as disclosed herein to the target locus of interest. In some embodiments, the PAM may be a 5′ PAM (i.e., located upstream of the 5′ end of the protospacer). In other embodiments, the PAM may be a 3′ PAM (i.e., located downstream of the 5′ end of the protospacer). In other embodiments, both a 5′ PAM and a 3′ PAM are required. In certain embodiments of the present disclosure, a PAM or PAM-like motif may not be required for directing binding of the effector protein (e.g. a Cas protein). In certain embodiments, a 5′ PAM is D (e.g., A, G, or U). In certain embodiments, a 5′ PAM is D for Cas9. In certain embodiments of the present disclosure, cleavage at repeat sequences may generate crRNAs (e.g. short or long crRNAs) containing a full spacer sequence flanked by a short nucleotide (e.g. 5, 6, 7, 8, 9, or 10 nt or longer if it is a dual repeat) repeat sequence at the 5′ end (this may be referred to as a crRNA “tag”) and the rest of the repeat at the 3′ end. In certain embodiments, targeting by the effector proteins described herein may require the lack of homology between the crRNA tag and the target 5′ flanking sequence. This requirement may be similar to that described further in Samai et al. “Co-transcriptional DNA and RNA Cleavage during Type III CRISPR-Cas Immunity” Cell 161, 1164-1174, May 21, 2015, where the requirement is thought to distinguish between bona fide targets on invading nucleic acids from the CRISPR array itself, and where the presence of repeat sequences will lead to full homology with the crRNA tag and prevent autoimmunity.

In certain embodiments, Cas protein is engineered and can comprise one or more mutations that reduce or eliminate nuclease activity, thereby reducing or eliminating RNA interfering activity. Mutations can also be made at neighboring residues, e.g., at amino acids near those that participate in the nuclease activity. In some embodiments, one or more putative catalytic nuclease domains are inactivated and the effector protein complex lacks cleavage activity and functions as an RNA binding complex. In a preferred embodiment, the resulting RNA binding complex may be linked with one or more functional domains as described herein.

In certain embodiments, the one or more functional domains are controllable, e.g. inducible.

In certain embodiments of the present disclosure, the guide RNA or mature crRNA comprises, consists essentially of, or consists of a direct repeat sequence and a guide sequence or spacer sequence. In certain embodiments, the guide RNA or mature crRNA comprises, consists essentially of, or consists of a direct repeat sequence linked to a guide sequence or spacer sequence. In preferred embodiments of the present disclosure, the mature crRNA comprises a stem loop or an optimized stem loop structure or an optimized secondary structure. In preferred embodiments the mature crRNA comprises a stem loop or an optimized stem loop structure in the direct repeat sequence, wherein the stem loop or optimized stem loop structure is important for cleavage activity. In certain embodiments, the mature crRNA preferably comprises a single stem loop. In certain embodiments, the direct repeat sequence preferably comprises a single stem loop. In certain embodiments, the cleavage activity of the effector protein complex is modified by introducing mutations that affect the stem loop RNA duplex structure. In preferred embodiments, mutations which maintain the RNA duplex of the stem loop may be introduced, whereby the cleavage activity of the effector protein complex is maintained. In other preferred embodiments, mutations which disrupt the RNA duplex structure of the stem loop may be introduced, whereby the cleavage activity of the effector protein complex is completely abolished.

The CRISPR system as provided herein can make use of a crRNA or analogous polynucleotide comprising a guide sequence, wherein the polynucleotide is an RNA, a DNA or a mixture of RNA and DNA, and/or wherein the polynucleotide comprises one or more nucleotide analogs. The sequence can comprise any structure, including but not limited to a structure of a native crRNA, such as a bulge, a hairpin or a stem loop structure. In certain embodiments, the polynucleotide comprising the guide sequence forms a duplex with a second polynucleotide sequence which can be an RNA or a DNA sequence.

The present disclosure also provides cells, tissues, organisms comprising the engineered Cas protein, the CRISPR-Cas systems, the polynucleotides encoding one or more components of the CRISPR-Cas systems, and/or vectors comprising the polynucleotides. The present disclosure also provides for the nucleotide sequence encoding the effector protein being codon optimized for expression in a eukaryote or eukaryotic cell in any of the herein described methods or compositions. In an embodiment of the present disclosure, the codon optimized effector protein is any Cas protein discussed herein and is codon optimized for operability in a eukaryotic cell or organism, e.g., such cell or organism as elsewhere herein mentioned, for instance, without limitation, a yeast cell, or a mammalian cell or organism, including a mouse cell, a rat cell, and a human cell or non-human eukaryote organism, e.g., plant.

In certain embodiments of the present disclosure, at least one nuclear localization signal (NLS) is attached to the nucleic acid sequences encoding the Cas proteins. In preferred embodiments at least one or more C-terminal or N-terminal NLSs are attached (and hence nucleic acid molecule(s) coding for the Cas protein can include coding for NLS(s) so that the expressed product has the NLS(s) attached or connected). In a preferred embodiment a C-terminal NLS is attached for optimal expression and nuclear targeting in eukaryotic cells, preferably human cells. The present disclosure also encompasses methods for delivering multiple nucleic acid components, wherein each nucleic acid component is specific for a different target locus of interest thereby modifying multiple target loci of interest. The nucleic acid component of the complex may comprise one or more protein-binding RNA aptamers. The one or more aptamers may be capable of binding a bacteriophage coat protein.

In a further aspect, the present disclosure provides a eukaryotic cell comprising a modified target locus of interest, wherein the target locus of interest has been modified according to in any of the herein described methods. A further aspect provides a cell line of said cell. Another aspect provides a multicellular organism comprising one or more said cells.

In certain embodiments, the modification of the target locus of interest may result in: the eukaryotic cell comprising altered expression of at least one gene product; the eukaryotic cell comprising altered expression of at least one gene product, wherein the expression of the at least one gene product is increased; the eukaryotic cell comprising altered expression of at least one gene product, wherein the expression of the at least one gene product is decreased; or the eukaryotic cell comprising an edited genome.

In certain embodiments, the eukaryotic cell may be a mammalian cell or a human cell.

In further embodiments, the non-naturally occurring or engineered compositions, the vector systems, or the delivery systems as described in the present specification may be used for: site-specific gene knockout; site-specific genome editing; RNA sequence-specific interference; or multiplexed genome engineering.

Also provided is a gene product from the cell, the cell line, or the organism as described herein. In certain embodiments, the amount of gene product expressed may be greater than or less than the amount of gene product from a cell that does not have altered expression or edited genome. In certain embodiments, the gene product may be altered in comparison with the gene product from a cell that does not have altered expression or edited genome.

In another aspect, the present disclosure provides a method for identifying novel nucleic acid modifying effectors, comprising: identifying putative nucleic acid modifying loci from a set of nucleic acid sequences encoding the putative nucleic acid modifying enzyme loci that are within a defined distance from a conserved genomic element of the loci, that comprise at least one protein above a defined size limit, or both; grouping the identified putative nucleic acid modifying loci into subsets comprising homologous proteins; identifying a final set of candidate nucleic acid modifying loci by selecting nucleic acid modifying loci from one or more subsets based on one or more of the following; subsets comprising loci with putative effector proteins with low domain homology matches to known protein domains relative to loci in other subsets, subsets comprising putative proteins with minimal distances to the conserved genomic element relative to loci in other subsets, subsets with loci comprising large effector proteins having a same orientations as putative adjacent accessory proteins relative to large effector proteins in other subsets, subset comprising putative effector proteins with lower existing nucleic acid modifying classifications relative to other loci, subsets comprising loci with a lower proximity to known nucleic acid modifying loci relative to other subsets, and total number of candidate loci in each subset.

In one embodiment, the set of nucleic acid sequences is obtained from a genomic or metagenomic database, such as a genomic or metagenomic database comprising prokaryotic genomic or metagenomic sequences.

In one embodiment, the defined distance from the conserved genomic element is between 1 kb and 25 kb.

In one embodiment, the conserved genomic element comprises a repetitive element, such as a CRISPR array. In a specific embodiment, the defined distance from the conserved genomic element is within 10 kb of the CRISPR array.

In one embodiment, the defined size limit of a protein comprised within the putative nucleic acid modifying (effector) locus is greater than 200 amino acids, or more particularly, the defined size limit is greater than 700 amino acids. In one embodiment, the putative nucleic acid modifying locus is between 900 to 1800 amino acids.

In one embodiment, the conserved genomic elements are identified using a repeat or pattern finding analysis of the set of nucleic acids, such as PILER-CR.

In one embodiment, the grouping step of the method described herein is based, at least in part, on results of a domain homology search or an HHpred protein domain homology search.

In one embodiment, the defined threshold is a BLAST nearest-neighbor cut-off value of 0 to 1e-7.

In one embodiment, the method described herein further comprises a filtering step that includes only loci with putative proteins between 900 and 1800 amino acids.

In one embodiment, the method described herein further comprises experimental validation of the nucleic acid modifying function of the candidate nucleic acid modifying effectors comprising generating a set of nucleic acid constructs encoding the nucleic acid modifying effectors and performing one or more biochemical validation assays, such as through the use of PAM validation in bacterial colonies, in vitro cleavage assays, the Surveyor method, experiments in mammalian cells, PAM validation, or a combination thereof.

In one embodiment, the method described herein further comprises preparing a non-naturally occurring or engineered composition comprising one or more proteins from the identified nucleic acid modifying loci.

In one embodiment, the identified loci comprise a Class 2 CRISPR effector, or the identified loci lack Cas1 or Cas2, or the identified loci comprise a single effector.

In one embodiment, the identified loci further comprise one or two small putative accessory proteins within 2 kb to 10 kb of the CRISPR array.

In one embodiment, a small accessory protein is less than 700 amino acids. In one embodiment, the small accessory protein is from 50 to 300 amino acids in length.

In one embodiment, the loci comprise no additional proteins out to 25 kb from the CRISPR array.

In one embodiment, the CRISPR array comprises direct repeat sequences comprising about 36 nucleotides in length. In a specific embodiment, the direct repeat comprises a GTTG/GUUG at the 5′ end that is reverse complementary to a CAAC at the 3′ end.

In one embodiment, the CRISPR array comprises spacer sequences comprising about 30 nucleotides in length.

In one embodiment, the identified loci lack a small accessory protein.

The present disclosure provides a method of identifying novel CRISPR effectors, comprising: a) identifying sequences in a genomic or metagenomic database encoding a CRISPR array; b) identifying one or more Open Reading Frames (ORFs) in said selected sequences within 10 kb of the CRISPR array; c) selecting loci based on the presence of a putative CRISPR effector protein between 900-1800 amino acids in size, d) selecting loci encoding a putative accessory protein of 50-300 amino acids; and e) identifying loci encoding a putative CRISPR effector and CRISPR accessory proteins and optionally classifying them based on structure analysis.

In one embodiment, the CRISPR effector is a Type II CRISPR effector. In an embodiment, step (a) comprises i) comparing sequences in a genomic and/or metagenomic database with at least one pre-identified seed sequence that encodes a CRISPR array, and selecting sequences comprising said seed sequence; or ii) identifying CRISPR arrays based on a CRISPR algorithm.

In an embodiment, step (d) comprises identifying nuclease domains. In an embodiment, step (d) comprises identifying RuvC and/or HPN domains.

In an embodiment, no ORF encoding Cast or Cas2 is present within 10 kb of the CRISPR array

In an embodiment, an ORF in step (b) encodes a putative accessory protein of 50-300 amino acids.

In an embodiment, putative novel CRISPR effectors obtained in step (d) are used as seed sequences for further comparing genomic and/or metagenomics sequences and subsequent selecting loci of interest as described in steps a) to d) of claim 1. In an embodiment, the pre-identified seed sequence is obtained by a method comprising: (a) identifying CRISPR motifs in a genomic or metagenomic database, (b) extracting multiple features in said identified CRISPR motifs, (c) classifying the CRISPR loci using unsupervised learning, (d) identifying conserved locus elements based on said classification, and (e) selecting therefrom a putative CRISPR effector suitable as seed sequence.

In an embodiment, the features include protein elements, repeat structure, repeat sequence, spacer sequence and spacer mapping. In an embodiment, the genomic and metagenomic databases are bacterial and/or archaeal genomes. In an embodiment, the genomic and metagenomic sequences are obtained from the Ensembl and/or NCBI genome databases. In an embodiment, the structure analysis in step (d) is based on secondary structure prediction and/or sequence alignments. In an embodiment, step (d) is achieved by clustering of the remaining loci based on the proteins they encode and manual curation of the obtained clusters.

In another aspect, the disclosure provides a method of altering activity of a Cas protein, comprising: identifying one or more candidate amino acids in the Cas protein based on a three-dimensional structure of at least a portion of the Cas protein, wherein the one or more candidate amino acids interact with a guide RNA that forms a complex with the Cas protein, or are in an inter-domain linker domain, or a bridge helix domain of the Cas protein; and mutating the one or more candidate amino acids thereby generating a mutated Cas protein, wherein activity the mutated Cas protein is different than the Cas protein.

### Small Cas Proteins and Orthologs

In some embodiments, the Cas proteins are a subgroup of Type II Cas proteins that are less than 850 amino acid in size. In some examples, the small Cas proteins are Type II-B or Type II-C Cas9 or Cas9-t.

The systems and compositions may comprise orthologs and homologs of the small Cas proteins. The terms “ortholog” and “homolog” are well known in the art. By means of further guidance, a “homolog” of a protein as used herein is a protein of the same species which performs the same or a similar function as the protein it is a homolog thereof. Homologous proteins may but need not be structurally related, or are only partially structurally related. An “ortholog” of a protein as used herein is a protein of a different species which performs the same or a similar function as the protein it is an ortholog of. Orthologous proteins may but need not be structurally related, or are only partially structurally related. Homologs and orthologs may be identified by homology modelling (see, e.g., Greer, Science vol. 228 (1985) 1055, and Blundell et al. Eur J Biochem vol 172 (1988), 513) or “structural BLAST” (Dey F, Cliff Zhang Q, Petrey D, Honig B. Toward a “structural BLAST”: using structural relationships to infer function. Protein Sci. 2013 April; 22(4):359-66. doi: 10.1002/pro.2225.). See also Shmakov et al. (2015) for application in the field of CRISPR-Cas loci. Homologous proteins may but need not be structurally related, or are only partially structurally related.

In particular embodiments, the homolog or ortholog of a Cas9 protein as referred to herein has a sequence homology or identity of at least 60%, preferably at least 70%, preferably at least 80%, more preferably at least 85%, even more preferably at least 90%, such as for instance at least 95% with a Cas proteins set forth in Table 12 herein.

The Cas9 gene is found in several diverse bacterial genomes, typically in the same locus with cast, cas2, and cas4 genes and a CRISPR cassette. Furthermore, the Cas9 protein contains a readily identifiable C-terminal region that is homologous to the transposon ORF-B and includes an active RuvC-like nuclease, an arginine-rich region.

In particular embodiments, the effector protein is a Cas9 effector protein from or originated from an organism from a genus comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacte, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus, Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, or Campylobacter

In further particular embodiments, the Cas9 effector protein is from or originated from an organism selected from S. mutans, S. agalactiae, S. equisimilis, S. sanguinis, S. pneumonia, C. jejuni, C. coli; N. salsuginis, N. tergarcus; S. auricularis, S. carnosus; N. meningitides, N. gonorrhoeae, L. monocytogenes, L. ivanovii; C. botulinum, C. difficile, C. tetani, or C. sordellii, Francisella tularensis 1, Francisella tularensis subsp. novicida, Prevotella albensis, Lachnospiraceae bacterium MC2017 1, Butyrivibrio proteoclasticus, Peregrinibacteria bacterium GW2011 GWA2_33_10, Parcubacteria bacterium GW2011 GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, and Porphyromonas macacae. In particular embodiments, the effector protein is a Cas9 effector protein from an organism from or originated from Streptococcus pyogenes, Staphylococcus aureus, or Streptococcus thermophilus Cas9. In a more preferred embodiment, the Cas9 is derived from a bacterial species selected from Streptococcus pyogenes, Staphylococcus aureus, or Streptococcus thermophilus Cas9. In certain embodiments, the Cas9 is derived from a bacterial species selected from Francisella tularensis 1, Prevotella albensis, Lachnospiraceae bacterium MC2017 1, Butyrivibrio proteoclasticus, Peregrinibacteria bacterium GW2011 GWA2_33_10, Parcubacteria bacterium GW2011 GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae. In certain embodiments, the Cas9p is derived from a bacterial species selected from Acidaminococcus sp. BV3L6, Lachnospiraceae bacterium MA2020. In certain embodiments, the effector protein is derived from a subspecies of Francisella tularensis 1, including but not limited to Francisella tularensis subsp. Novicida

The effector protein may comprise a chimeric effector protein comprising a first fragment from a first effector protein (e.g., a Cas9) ortholog and a second fragment from a second effector (e.g., a Cas9) protein ortholog, and wherein the first and second effector protein orthologs are different. At least one of the first and second effector protein (e.g., a Cas9) orthologs may comprise an effector protein (e.g., a Cas9) from an organism comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus; e.g., a chimeric effector protein comprising a first fragment and a second fragment wherein each of the first and second fragments is selected from a Cas9 of an organism comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus wherein the first and second fragments are not from the same bacteria; for instance a chimeric effector protein comprising a first fragment and a second fragment wherein each of the first and second fragments is selected from a Cas9 of S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii; Francisella tularensis 1, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae, wherein the first and second fragments are not from the same bacteria.

As used herein, when a Cas protein originates form a species, it may be the wild type Cas protein in the species, or a homolog of the wild type Cas protein in the species. The Cas protein that is a homolog of the wild type Cas protein in the species may comprise one or more variations (e.g., mutations, truncations, etc.) of the wild type Cas protein.

It will be appreciated that any of the functionalities described herein may be engineered into Cas proteins from other orthologs, including chimeric enzymes comprising fragments from multiple orthologs. A chimeric enzyme can comprise a first fragment and a second fragment, and the fragments can be of CRISPR enzyme orthologs of organisms of genuses herein mentioned or of species herein mentioned; advantageously the fragments are from CRISPR enzyme orthologs of different species.

In embodiments, the systems and compositions herein also encompass a functional variant of the effector protein or a homologue or an orthologue thereof. A “functional variant” of a protein as used herein refers to a variant of such protein which retains at least partially the activity of that protein. Functional variants may include mutants (which may be insertion, deletion, or replacement mutants), including polymorphs. Also included within functional variants are fusion products of such protein with another, usually unrelated, nucleic acid, protein, polypeptide or peptide. Functional variants may be naturally occurring or may be man-made. In an embodiment, nucleic acid molecule(s) encoding the Cas proteins, or an ortholog or homolog thereof, may be codon-optimized for expression in an eukaryotic cell. A eukaryote can be as herein discussed. Nucleic acid molecule(s) can be engineered or non-naturally occurring.

In an embodiment, the Cas protein or an ortholog or homolog thereof, may comprise one or more mutations. The mutations may be artificially introduced mutations and may include but are not limited to one or more mutations in a catalytic domain, e.g., one or more mutations are introduced into one or more of the RuvC and/or HNH domains.

In an embodiment, the Cas protein or an ortholog or homolog thereof, may be used as a generic nucleic acid binding protein with fusion to or being operably linked to a functional domain. Exemplary functional domains may include but are not limited to translational initiator, translational activator, translational repressor, nucleases, in particular ribonucleases, a spliceosome, beads, a light inducible/controllable domain or a chemically inducible/controllable domain.

### Cas Variants

The Cas proteins herein include variants and mutated forms of Cas proteins (comparing to wildtype or naturally occurring Cas proteins). In some examples, the present disclosure includes variants and mutated forms of the small Cas proteins. The variants or mutated forms of Cas protein may be catalytically inactive, e.g., have no or reduced nuclease activity compared to a corresponding wildtype. In certain examples, the variants or mutated forms of Cas protein have nickase activity.

In some cases, the present disclosure provides for mutated small Cas proteins comprising one or more modified of amino acids. The amino acids: (a) interact with a guide RNA that forms a complex with the mutated Cas protein; (b) are in an active site, an inter-domain linker domain, or a bridge helix domain of the mutated Cas protein; or (c) a combination thereof.

The term “corresponding amino acid” or “residue which corresponds to” refers to a particular amino acid or analogue thereof in a Cas homolog or ortholog that is identical or functionally equivalent to an amino acid in reference Cas protein. Accordingly, as used herein, referral to an “amino acid position corresponding to amino acid position [X]” of a specified Cas protein represents referral to a collection of equivalent positions in other recognized Cas and structural homologues and families.

### Structural (Sub)Domains

In another aspect, the disclosure provides a mutated Cas protein comprising one or more mutations of amino acids, wherein the amino acids: interact with a guide RNA that forms a complex with the engineered Cas protein; or are in an active site, e.g., in RuvC and/or HNH domains.

The types of mutations can be conservative mutations or non-conservative mutations. In certain preferred embodiments, the amino acid which is mutated is mutated into alanine (A). In certain preferred embodiments, if the amino acid to be mutated is an aromatic amino acid, it is mutated into alanine or another aromatic amino acid (e.g. H, Y, W, or F). In certain preferred embodiments, if the amino acid to be mutated is a charged amino acid, it is mutated into alanine or another charged amino acid (e.g. H, K, R, D, or E). In certain preferred embodiments, if the amino acid to be mutated is a charged amino acid, it is mutated into alanine or another charged amino acid having the same charge. In certain preferred embodiments, if the amino acid to be mutated is a charged amino acid, it is mutated into alanine or another charged amino acid having the opposite charge.

The present disclosure also provides for methods and compositions wherein one or more amino acid residues of the effector protein may be modified e.g., an engineered or non-naturally-occurring effector protein or Cas. In an embodiment, the modification may comprise mutation of one or more amino acid residues of the effector protein. The one or more mutations may be in one or more catalytically active domains of the effector protein, or a domain interacting with the crRNA (such as the guide sequence or direct repeat sequence). The effector protein may have reduced or abolished nuclease activity or alternatively increased nuclease activity compared with an effector protein lacking said one or more mutations. The effector protein may not direct cleavage of the RNA strand at the target locus of interest. In a preferred embodiment, the one or more mutations may comprise two mutations.

The Cas protein herein may comprise one or more amino acids mutated. In some embodiments, the amino acid is mutated to A, P, or V, preferably A. In some embodiments, the amino acid is mutated to a hydrophobic amino acid. In some embodiments, the amino acid is mutated to an aromatic amino acid. In some embodiments, the amino acid is mutated to a charged amino acid. In some embodiments, the amino acid is mutated to a positively charged amino acid. In some embodiments, the amino acid is mutated to a negatively charged amino acid. In some embodiments, the amino acid is mutated to a polar amino acid. In some embodiments, the amino acid is mutated to an aliphatic amino acid.

### Destabilized Cas and Fusion Proteins

In certain embodiments, the Cas protein according to the present disclosure as described herein is associated with or fused to a destabilization domain (DD). In some embodiments, the DD is ER50. A corresponding stabilizing ligand for this DD is, in some embodiments, 4HT. As such, in some embodiments, one of the at least one DDs is ER50 and a stabilizing ligand therefor is 4HT or CMP8. In some embodiments, the DD is DHFR50. A corresponding stabilizing ligand for this DD is, in some embodiments, TMP. As such, in some embodiments, one of the at least one DDs is DHFR50 and a stabilizing ligand therefor is TMP. In some embodiments, the DD is ER50. A corresponding stabilizing ligand for this DD is, in some embodiments, CMP8. CMP8 may therefore be an alternative stabilizing ligand to 4HT in the ER50 system. While it may be possible that CMP8 and 4HT can/should be used in a competitive matter, some cell types may be more susceptible to one or the other of these two ligands, and from this disclosure and the knowledge in the art the skilled person can use CMP8 and/or 4HT.

In some embodiments, one or two DDs may be fused to the N-terminal end of the Cas with one or two DDs fused to the C-terminal of the Cas. In some embodiments, the at least two DDs are associated with the Cas and the DDs are the same DD, i.e. the DDs are homologous. Thus, both (or two or more) of the DDs could be ER50 DDs. This is preferred in some embodiments. Alternatively, both (or two or more) of the DDs could be DHFR50 DDs. This is also preferred in some embodiments. In some embodiments, the at least two DDs are associated with the Cas and the DDs are different DDs, i.e. the DDs are heterologous. Thus, one of the DDS could be ER50 while one or more of the DDs or any other DDs could be DHFR50. Having two or more DDs which are heterologous may be advantageous as it would provide a greater level of degradation control. A tandem fusion of more than one DD at the N or C-term may enhance degradation; and such a tandem fusion can be, for example ER50-ER50-Cas or DHFR-DHFR-Cas It is envisaged that high levels of degradation would occur in the absence of either stabilizing ligand, intermediate levels of degradation would occur in the absence of one stabilizing ligand and the presence of the other (or another) stabilizing ligand, while low levels of degradation would occur in the presence of both (or two of more) of the stabilizing ligands. Control may also be imparted by having an N-terminal ER50 DD and a C-terminal DHFR50 DD.

In some embodiments, the fusion of the Cas with the DD comprises a linker between the DD and the Cas. In some embodiments, the linker is a GlySer linker. In some embodiments, the DD-Cas further comprises at least one Nuclear Export Signal (NES). In some embodiments, the DD-Cas comprises two or more NESs. In some embodiments, the DD-Cas comprises at least one Nuclear Localization Signal (NLS). This may be in addition to an NES. In some embodiments, the Cas comprises or consists essentially of or consists of a localization (nuclear import or export) signal as, or as part of, the linker between the Cas and the DD. HA or Flag tags are also within the ambit of the present disclosure as linkers. Applicants use NLS and/or NES as linker and also use Glycine Serine linkers as short as GS up to (GGGGS)₃ (SEQ ID NO: 17).

Destabilizing domains have general utility to confer instability to a wide range of proteins; see, e.g., Miyazaki, J Am Chem Soc. Mar. 7, 2012; 134(9): 3942-3945, incorporated herein by reference. CMP8 or 4-hydroxytamoxifen can be destabilizing domains. More generally, A temperature-sensitive mutant of mammalian DHFR (DHFRts), a destabilizing residue by the N-end rule, was found to be stable at a permissive temperature but unstable at 37° C. The addition of methotrexate, a high-affinity ligand for mammalian DHFR, to cells expressing DHFRts inhibited degradation of the protein partially. This was an important demonstration that a small molecule ligand can stabilize a protein otherwise targeted for degradation in cells. A rapamycin derivative was used to stabilize an unstable mutant of the FRB domain of mTOR (FRB*) and restore the function of the fused kinase, GSK-3β.6,7 This system demonstrated that ligand-dependent stability represented an attractive strategy to regulate the function of a specific protein in a complex biological environment. A system to control protein activity can involve the DD becoming functional when the ubiquitin complementation occurs by rapamycin induced dimerization of FK506-binding protein and FKBP12. Mutants of human FKBP12 or ecDHFR protein can be engineered to be metabolically unstable in the absence of their high-affinity ligands, Shield-1 or trimethoprim (TMP), respectively. These mutants are some of the possible destabilizing domains (DDs) useful in the practice of the present disclosure and instability of a DD as a fusion with a Cas confers to the Cas degradation of the entire fusion protein by the proteasome. Shield-1 and TMP bind to and stabilize the DD in a dose-dependent manner. The estrogen receptor ligand binding domain (ERLBD, residues 305-549 of ERS1) can also be engineered as a destabilizing domain. Since the estrogen receptor signaling pathway is involved in a variety of diseases such as breast cancer, the pathway has been widely studied and numerous agonist and antagonists of estrogen receptor have been developed. Thus, compatible pairs of ERLBD and drugs are known. There are ligands that bind to mutant but not wild-type forms of the ERLBD. By using one of these mutant domains encoding three mutations (L384M, M421G, G521R)12, it is possible to regulate the stability of an ERLBD-derived DD using a ligand that does not perturb endogenous estrogen-sensitive networks. An additional mutation (Y537S) can be introduced to further destabilize the ERLBD and to configure it as a potential DD candidate. This tetra-mutant is an advantageous DD development. The mutant ERLBD can be fused to a Cas and its stability can be regulated or perturbed using a ligand, whereby the Cas has a DD. Another DD can be a 12-kDa (107-amino-acid) tag based on a mutated FKBP protein, stabilized by Shield1 ligand; see, e.g., Nature Methods 5, (2008). For instance, a DD can be a modified FK506 binding protein 12 (FKBP12) that binds to and is reversibly stabilized by a synthetic, biologically inert small molecule, Shield-1; see, e.g., Banaszynski L A, Chen L C, Maynard-Smith L A, Ooi A G, Wandless T J. A rapid, reversible, and tunable method to regulate protein function in living cells using synthetic small molecules. Cell. 2006; 126:995-1004; Banaszynski L A, Sellmyer M A, Contag C H, Wandless T J, Thorne S H. Chemical control of protein stability and function in living mice. Nat Med. 2008; 14:1123-1127; Maynard-Smith L A, Chen L C, Banaszynski L A, Ooi A G, Wandless T J. A directed approach for engineering conditional protein stability using biologically silent small molecules. The Journal of biological chemistry. 2007; 282:24866-24872; and Rodriguez, Chem Biol. Mar. 23, 2012; 19(3): 391-398—all of which are incorporated herein by reference and may be employed in the practice of the present disclosure in selected a DD to associate with a Cas in the practice of this present disclosure. As can be seen, the knowledge in the art includes a number of DDs, and the DD can be associated with, e.g., fused to, advantageously with a linker, to a Cas, whereby the DD can be stabilized in the presence of a ligand and when there is the absence thereof the DD can become destabilized, whereby the Cas is entirely destabilized, or the DD can be stabilized in the absence of a ligand and when the ligand is present the DD can become destabilized; the DD allows the Cas and hence the CRISPR-Cas complex or system to be regulated or controlled—turned on or off so to speak, to thereby provide means for regulation or control of the system, e.g., in an in vivo or in vitro environment. For instance, when a protein of interest is expressed as a fusion with the DD tag, it is destabilized and rapidly degraded in the cell, e.g., by proteasomes. Thus, absence of stabilizing ligand leads to a D associated Cas being degraded. When a new DD is fused to a protein of interest, its instability is conferred to the protein of interest, resulting in the rapid degradation of the entire fusion protein. Peak activity for Cas is sometimes beneficial to reduce off-target effects. Thus, short bursts of high activity are preferred. The present disclosure is able to provide such peaks. In some senses the system is inducible. In some other senses, the system repressed in the absence of stabilizing ligand and de-repressed in the presence of stabilizing ligand.

### Deactivated/Inactivated/Dead Cas Proteins

In certain embodiments, the Cas protein herein is a catalytically inactive or dead Cas protein. In some cases, Cas protein herein is a catalytically inactive or dead Cas protein (dCas). In some cases, a dead Cas protein, e.g., a dead Cas protein has nickase activity. In some embodiments, the dCas protein comprises mutations in the nuclease domain. In some embodiments, the dCas protein has been truncated. In some cases, the dead Cas proteins may be fused with a deaminase herein, e.g., an adenosine deaminase.

Where the Cas9 protein has nuclease activity, the Cas9 protein may be modified to have diminished nuclease activity e.g., nuclease inactivation of at least 70%, at least 80%, at least 90%, at least 95%, at least 97%, or 100% as compared with the wild type enzyme; or to put in another way, a Cas9 enzyme having advantageously about 0% of the nuclease activity of the non-mutated or wild type Cas9 enzyme or CRISPR enzyme, or no more than about 3% or about 5% or about 10% of the nuclease activity of the non-mutated or wild type Cas9 enzyme. This is possible by introducing mutations into the nuclease domains of the Cas9 and orthologs thereof.

In certain embodiments, the CRISPR enzyme is engineered and can comprise one or more mutations that reduce or eliminate a nuclease activity. When the enzyme is not SpCas9, mutations may be made at any or all residues corresponding to positions 10, 762, 840, 854, 863 and/or 986 of SpCas9 (which may be ascertained for instance by standard sequence comparison tools). In particular, any or all of the following mutations are preferred in SpCas9: D10, E762, H840, N854, N863, or D986; as well as conservative substitution for any of the replacement amino acids is also envisaged. The point mutations to be generated to substantially reduce nuclease activity include but are not limited to D10A, E762A, H840A, N854A, N863A and/or D986A. In an aspect the present disclosure provides a herein-discussed composition, wherein the CRISPR enzyme comprises two or more mutations wherein two or more of D10, E762, H840, N854, N863, or D986 according to SpCas9 protein or any corresponding or N580 according to SaCas9 protein ortholog are mutated, or the CRISPR enzyme comprises at least one mutation wherein at least H840 is mutated. In an aspect the present disclosure provides a herein-discussed composition wherein the CRISPR enzyme comprises two or more mutations comprising D10A, E762A, H840A, N854A, N863A or D986A according to SpCas9 protein or any corresponding ortholog, or N580A according to SaCas9 protein, or at least one mutation comprising H840A, or, optionally wherein the CRISPR enzyme comprises: N580A according to SaCas9 protein or any corresponding ortholog; or D 10A according to SpCas9 protein, or any corresponding ortholog, and N580A according to SaCas9 protein. In an aspect the present disclosure provides a herein-discussed composition, wherein the CRISPR enzyme comprises H840A, or D 10A and H840A, or D 10A and N863A, according to SpCas9 protein or any corresponding ortholog.

Mutations can also be made at neighboring residues, e.g., at amino acids near those indicated above that participate in the nuclease activity. In some embodiments, only the RuvC domain is inactivated, and in other embodiments, another putative nuclease domain is inactivated, wherein the effector protein complex functions as a nickase and cleaves only one DNA strand. In a preferred embodiment, the other putative nuclease domain is a HincII-like endonuclease domain. In some embodiments, two Cas9 variants (each a different nickase) are used to increase specificity, two nickase variants are used to cleave DNA at a target (where both nickases cleave a DNA strand, while minimizing or eliminating off-target modifications where only one DNA strand is cleaved and subsequently repaired). In preferred embodiments the Cas9 effector protein cleaves sequences associated with or at a target locus of interest as a homodimer comprising two Cas9 effector protein molecules. In a preferred embodiment the homodimer may comprise two Cas9 effector protein molecules comprising a different mutation in their respective RuvC domains.

The inactivated Cas9 CRISPR enzyme may have associated (e.g., via fusion protein) one or more functional domains, including for example, one or more domains from the group comprising, consisting essentially of, or consisting of methylase activity, demethylase activity, transcription activation activity, transcription repression activity, transcription release factor activity, histone modification activity, RNA cleavage activity, DNA cleavage activity, nucleic acid binding activity, and molecular switches (e.g., light inducible). Preferred domains are Fok1, VP64, P65, HSF1, MyoD1. In the event that Fok1 is provided, it is advantageous that multiple Fok1 functional domains are provided to allow for a functional dimer and that gRNAs are designed to provide proper spacing for functional use (Fok1) as specifically described in Tsai et al. Nature Biotechnology, Vol. 32, Number 6, June 2014). The adaptor protein may utilize known linkers to attach such functional domains. In some cases, it is advantageous that additionally at least one NLS is provided. In some instances, it is advantageous to position the NLS at the N terminus. When more than one functional domain is included, the functional domains may be the same or different.

In general, the positioning of the one or more functional domain on the inactivated Cas9 enzyme is one which allows for correct spatial orientation for the functional domain to affect the target with the attributed functional effect. For example, if the functional domain is a transcription activator (e.g., VP64 or p65), the transcription activator is placed in a spatial orientation which allows it to affect the transcription of the target. Likewise, a transcription repressor will be advantageously positioned to affect the transcription of the target, and a nuclease (e.g., Fok1) will be advantageously positioned to cleave or partially cleave the target. This may include positions other than the N-/C-terminus of the CRISPR enzyme.

The dead or deactivated Cas proteins may be used as target-binding proteins, (e.g., DNA binding proteins). In these cases, the dead or deactivated Cas proteins may be fused with one or more functional domains.

As described herein, corresponding catalytic domains of a Cas9 effector protein may also be mutated to produce a mutated Cas9 effector protein lacking all DNA cleavage activity or having substantially reduced DNA cleavage activity. In some embodiments, a nucleic acid-targeting effector protein may be considered to substantially lack all RNA cleavage activity when the RNA cleavage activity of the mutated enzyme is about no more than 25%, 10%, 5%, 1%, 0.1%, 0.01%, or less of the nucleic acid cleavage activity of the non-mutated form of the enzyme; an example can be when the nucleic acid cleavage activity of the mutated form is nil or negligible as compared with the non-mutated form. An effector protein may be identified with reference to the general class of enzymes that share homology to the biggest nuclease with multiple nuclease domains from the Type II CRISPR system. Most preferably, the effector protein is Cas9. In further embodiments, the effector protein is a Type II protein. By derived, Applicants mean that the derived enzyme is largely based, in the sense of having a high degree of sequence homology with, a wildtype enzyme, but that it has been mutated (modified) in some way as known in the art or as described herein.

### Functional Domains

In some embodiments, one or more functional domains are associated with the Cas9 effector protein. In some embodiments, one or more functional domains are associated with an adaptor protein, for example as used with the modified guides of Konnerman et al. (Nature 517, 583-588, 29 Jan. 2015). In some embodiments, one or more functional domains are associated with a dead gRNA (dRNA). In some embodiments, a dRNA complex with active Cas9 effector protein directs gene regulation by a functional domain at on gene locus while an gRNA directs DNA cleavage by the active Cas9 effector protein at another locus, for example as described analogously in CRISPR-Cas9 systems by Dahlman et al., ‘Orthogonal gene control with a catalytically active Cas9 nuclease’ (in press). In some embodiments, dRNAs are selected to maximize selectivity of regulation for a gene locus of interest compared to off-target regulation. In some embodiments, dRNAs are selected to maximize target gene regulation and minimize target cleavage

For the purposes of the following discussion, reference to a functional domain could be a functional domain associated with the Cas9 effector protein or a functional domain associated with the adaptor protein.

In the practice of the present disclosure, loops of the gRNA may be extended, without colliding with the Cas9 protein by the insertion of distinct RNA loop(s) or distinct sequence(s) that may recruit adaptor proteins that can bind to the distinct RNA loop(s) or distinct sequence(s). The adaptor proteins may include but are not limited to orthogonal RNA-binding protein/aptamer combinations that exist within the diversity of bacteriophage coat proteins. A list of such coat proteins includes, but is not limited to: Qβ, F2, GA, fr, JP501, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s and PRR1. These adaptor proteins or orthogonal RNA binding proteins can further recruit effector proteins or fusions which comprise one or more functional domains. In some embodiments, the functional domain may be selected from the group consisting of: transposase domain, integrase domain, recombinase domain, resolvase domain, invertase domain, protease domain, DNA methyltransferase domain, DNA hydroxylmethylase domain, DNA demethylase domain, histone acetylase domain, histone deacetylases domain, nuclease domain, repressor domain, activator domain, nuclear-localization signal domains, transcription-regulatory protein (or transcription complex recruiting) domain, cellular uptake activity associated domain, nucleic acid binding domain, antibody presentation domain, histone modifying enzymes, recruiter of histone modifying enzymes; inhibitor of histone modifying enzymes, histone methyltransferase, histone demethylase, histone kinase, histone phosphatase, histone ribosylase, histone deribosylase, histone ubiquitinase, histone deubiquitinase, histone biotinase and histone tail protease. In some preferred embodiments, the functional domain is a transcriptional activation domain, such as, without limitation, VP64, p65, MyoD1, HSF1, RTA, SET7/9 or a histone acetyltransferase. In some embodiments, the functional domain is a transcription repression domain, preferably KRAB. In some embodiments, the transcription repression domain is SID, or concatemers of SID (e.g. SID4X). In some embodiments, the functional domain is an epigenetic modifying domain, such that an epigenetic modifying enzyme is provided. In some embodiments, the functional domain is an activation domain, which may be the P65 activation domain.

In some examples, the Cas9 is associated with a ligase or functional fragment thereof. The ligase may ligate a single-strand break (a nick) generated by the Cas9. In certain cases, the ligase may ligate a double-strand break generated by the Cas9. In certain examples, the Cas9 is associated with a reverse transcriptase or functional fragment thereof.

In some embodiments, the one or more functional domains is an NLS (Nuclear Localization Sequence) or an NES (Nuclear Export Signal). In some embodiments, the one or more functional domains is a transcriptional activation domain comprises VP64, p65, MyoD1, HSF1, RTA, SET7/9 and a histone acetyltransferase. Other references herein to activation (or activator) domains in respect of those associated with the CRISPR enzyme include any known transcriptional activation domain and specifically VP64, p65, MyoD1, HSF1, RTA, SET7/9 or a histone acetyltransferase.

In some embodiments, the one or more functional domains is a transcriptional repressor domain. In some embodiments, the transcriptional repressor domain is a KRAB domain. In some embodiments, the transcriptional repressor domain is a NuE domain, NcoR domain, SID domain or a SID4X domain.

In some embodiments, the one or more functional domains have one or more activities comprising methylase activity, demethylase activity, transcription activation activity, transcription repression activity, transcription release factor activity, histone modification activity, RNA cleavage activity, DNA cleavage activity, DNA integration activity or nucleic acid binding activity.

Histone modifying domains are also preferred in some embodiments. Exemplary histone modifying domains are discussed below. Transposase domains, HR (Homologous Recombination) machinery domains, recombinase domains, and/or integrase domains are also preferred as the present functional domains. In some embodiments, DNA integration activity includes HR machinery domains, integrase domains, recombinase domains and/or transposase domains. Histone acetyltransferases are preferred in some embodiments.

In some embodiments, the DNA cleavage activity is due to a nuclease. In some embodiments, the nuclease comprises a Fok1 nuclease. See, “Dimeric CRISPR RNA-guided Fok1 nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided Fok1 Nucleases that recognize extended sequences and can edit endogenous genes with high efficiencies in human cells.

In some embodiments, the one or more functional domains is attached to the Cas9 effector protein so that upon binding to the sgRNA and target the functional domain is in a spatial orientation allowing for the functional domain to function in its attributed function.

In some embodiments, the one or more functional domains is attached to the adaptor protein so that upon binding of the Cas9 effector protein to the gRNA and target, the functional domain is in a spatial orientation allowing for the functional domain to function in its attributed function.

In an aspect the present disclosure provides a composition as herein discussed wherein the one or more functional domains is attached to the Cas9 effector protein or adaptor protein via a linker, optionally a GlySer linker, as discussed herein.

In particular embodiments the Cas9 effector protein comprise one or more heterologous functional domains. The one or more heterologous functional domains may comprise one or more nuclear localization signal (NLS) domains. The one or more heterologous functional domains may comprise at least two or more NLSs. The one or more heterologous functional domains may comprise one or more transcriptional activation domains. A transcriptional activation domain may comprise VP64. The one or more heterologous functional domains may comprise one or more transcriptional repression domains. A transcriptional repression domain may comprise a KRAB domain or a SID domain. The one or more heterologous functional domain may comprise one or more nuclease domains. The one or more nuclease domains may comprise Fok1.

Functional domains may be used to regulate transcription, e.g., transcriptional repression. Transcriptional repression is often mediated by chromatin modifying enzymes such as histone methyltransferases (HMTs) and deacetylases (HDACs). Repressive histone effector domains are known and an exemplary list is provided below. In the exemplary table, preference was given to proteins and functional truncations of small size to facilitate efficient viral packaging (for instance via AAV). In general, however, the domains may include HDACs, hi stone methyltransferases (HMTs), and hi stone acetyltransferase (HAT) inhibitors, as well as HDAC and HMT recruiting proteins. The functional domain may be or include, in some embodiments, HDAC Effector Domains, HDAC Recruiter Effector Domains, Histone Methyltransferase (HMT) Effector Domains, Histone Methyltransferase (HMT) Recruiter Effector Domains, or Histone Acetyltransferase Inhibitor Effector Domains.

TABLE 1
TDAC Effector Domains

| | | | | | Full | | | |
|---|---|---|---|---|---|---|---|---|
| Subtype/ | | Substrate | Modification | | size | Selected | Final size | Catalytic |
| Complex | Name | (if known) | (if known) | Organism | (aa) | truncation (aa) | (aa) | domain |
| HDAC 1 | HDAC8 | — | — | X. laevis | 325 | 1-325 | 325 | 1-272: |
| | | | | | | | | HDAC |
| HDAC 1 | RPD3 | — | — | S. cerevisiae | 433 | 19-340 | 322 | 19-331: |
| | | | | | | | (Vannier) | HDAC |
| HDAC | MesoLo4 | — | — | M. loti | 300 | 1-300 | 300 | — |
| IV | | | | | | (Gregoretti) | | |
| HDAC | HDAC11 | — | — | H. sapiens | 347 | 1-347 (Gao) | 347 | 14-326: |
| IV | | | | | | | | HDAC |
| HD2 | HDT1 | — | — | A. thaliana | 245 | 1-211 (Wu) | 211 | — |
| SIRT I | SIRT3 | H3K9Ac | — | H. sapiens | 399 | 143-399 (Scher) | 257 | 126-382: |
| | | H4K16Ac | | | | | | SIRT |
| | | H3K56Ac | | | | | | |
| SIRT I | HST2 | — | — | C. albicans | 331 | 1-331 (Hnisz) | 331 | — |
| SIRT I | CobB | — | — | E. coli (K12) | 242 | 1-242 (Landry) | 242 | — |
| SIRT I | HST2 | — | — | S. cerevisiae | 357 | 8-298 (Wilson) | 291 | — |
| SIRT III | SIRT5 | H4K8Ac | — | H. sapiens | 310 | 37-310 (Gertz) | 274 | 41-309: |
| | | H4K16Ac | | | | | | SIRT |
| SIRT III | Sir2A | — | — | P. falciparum | 273 | 1-273 (Zhu) | 273 | 19-273: |
| | | | | | | | | SIRT |
| SIRT IV | SIRT6 | H3K9Ac | — | H. sapiens | 355 | 1-289 (Tennen) | 289 | 35-274: |
| | | H3K56Ac | | | | | | SIRT |

Accordingly, the repressor domains of the present disclosure may be selected from histone methyltransferases (HMTs), histone deacetylases (HDACs), histone acetyltransferase (HAT) inhibitors, as well as HDAC and HMT recruiting proteins.

The HDAC domain may be any of those in the table above, namely: HDAC8, RPD3, MesoLo4, HDAC11, HDT1, SIRT3, HST2, CobB, HST2, SIRT5, Sir2A, or SIRT6.

In some embodiment, the functional domain may be a HDAC Recruiter Effector Domain. Preferred examples include those in the Table 1 below, namely MeCP2, MBD2b, Sin3a, NcoR, SALL1, RCOR1. NcoR is exemplified in the present Examples and, although preferred, it is envisaged that others in the class will also be useful.

TABLE 2
HDAC Recruiter Effector Domains

| | | | | | Full | | Final | |
|---|---|---|---|---|---|---|---|---|
| Subtype/ | | Substrate | Modification | | size | Selected | size | |
| Complex | Name | (if known) | (if known) | Organism | (aa) | truncation (aa) | (aa) | Catalytic domain |
| Sin3a | MeCP2 | — | — | R. norvegicus | 492 | 207-492 (Nan) | 286 | — |
| Sin3a | MBD2b | — | — | H. sapiens | 262 | 45-262 (Boeke) | 218 | — |
| Sin3a | Sin3a | — | — | H. sapiens | 1273 | 524-851 | 328 | 627-829: HDAC1 |
| | | | | | | (Laherty) | | interaction |
| NcoR | NcoR | — | — | H. sapiens | 2440 | 420-488 (Zhang) | 69 | — |
| NuRD | SALL1 | — | — | M. musculus | 1322 | 1-93 (Lauberth) | 93 | — |
| CoREST | RCOR1 | — | — | H. sapiens | 482 | 81-300 (Gu, | 220 | — |
| | | | | | | Ouyang) | | |

In some embodiments, the functional domain may be a Methyltransferase (HMT) Effector Domain. Preferred examples include those in the Table below, namely NUE, vSET, EHMT2/G9A, SUV39H1, dim-5, KYP, SUVR4, SET4, SET1, SETD8, and TgSET8. NUE is exemplified in the present Examples and, although preferred, it is envisaged that others in the class will also be useful.

TABLE 3
Histone Methyltransferase (HMT) Effector Domains

| | | Substrate | | | | Selected | | |
|---|---|---|---|---|---|---|---|---|
| Subtype/ | | (if | Modification | | Full size | truncation | Final | |
| Complex | Name | known) | (if known) | Organism | (aa) | (aa) | size (aa) | Catalytic domain |
| SET | NUE | H2B, H3, | — | C. trachomatis | 219 | 1-219 | 219 | — |
| | | H4 | | | | (Pennini) | | |
| SET | vSET | — | H3K27me3 | P. bursaria | 119 | 1-119 | 119 | 4-112: SET2 |
| | | | | chlorella virus | | (Mujtaba) | | |
| SUV39 | EHMT2/ | H1.4K2, | H3K9me1/2, | M. musculus | 1263 | 969-1263 | 295 | 1025-1233: |
| family | G9A | H3K9, | H1K25me1 | | | (Tachibana) | | preSET, SET, |
| | | H3K27 | | | | | | postSET |
| SUV39 | SUV39 | — | H3K9me2/3 | H. sapiens | 412 | 79-412 | 334 | 172-412: |
| | H1 | | | | | (Snowden) | | preSET, SET, |
| | | | | | | | | postSET |
| Suvar3-9 | dim-5 | — | H3K9me3 | N. crassa | 331 | 1-331 | 331 | 77-331: preSET, |
| | | | | | | (Rathert) | | SET, postSET |
| Suvar3-9 | KYP | — | H3K9me1/2 | A. thaliana | 624 | 335-601 | 267 | — |
| (SUVH | | | | | | | (Jackson) | |
| subfamily) | | | | | | | | |
| Suvar3-9 | SUVR4 | H3K9me1 | H3K9me2/3 | A. thaliana | 492 | 180-492 | 313 | 192-462: |
| (SUVR | | | | | | | (Thorstensen) | preSET, SET, |
| subfamily) | | | | | | | | postSET |
| Suvar4-20 | SET4 | — | H4K20me3 | C. elegans | 288 | 1-288 (Vielle) | 288 | — |
| SET8 | SET1 | — | H4K20me1 | C. elegans | 242 | 1-242 (Vielle) | 242 | — |
| SET8 | SETD8 | — | H4K20me1 | H. sapiens | 393 | 185-393 | 209 | 256-382: SET |
| | | | | | | | (Couture) | |
| SET8 | TgSET8 | — | H4K20me1/ | T. gondii | 1893 | 1590-1893 | 304 | 1749-1884: SET |
| | | | 2/3 | | | (Sautel) | | |

In some embodiments, the functional domain may be a Histone Methyltransferase (HMT) Recruiter Effector Domain. Preferred examples include those in the Table below, namely Hp1a, PHF19, and NIPP1.

TABLE 4
Histone Methyltransferase (HMT) Recruiter Effector Domains

| | | | | | Full | Selected | | |
|---|---|---|---|---|---|---|---|---|
| Subtype/ | | Substrate | Modification | | size | truncation | Final size | |
| Complex | Name | (if known) | (if known) | Organism | (aa) | (aa) | (aa) | Catalytic domain |
| — | Hp1a | — | H3K9me3 | M. musculus | 191 | 73-191 | 119 | 121-179: |
| | | | | | | | (Hathaway) | chromoshadow |
| — | PHF19 | — | H3K27me3 | H. sapiens | 580 | (1-250) + | 335 (Ballaré) | 163-250: PHD2 |
| | | | | | | GGSG | | |
| | | | | | | linker + | | |
| | | | | | | (500-580) | | |
| — | NIPP1 | — | H3K27me3 | H. sapiens | 351 | 1-329 (Jin) | 329 | 310-329: EED |

In some embodiments, the functional domain may be Histone Acetyltransferase Inhibitor Effector Domain. Preferred examples include SET/TAF-1β listed in the Table below.

TABLE 5
Histone Acetyltransferase Inhibitor Effector Domains

| | | | | | Full | Selected | Final | |
|---|---|---|---|---|---|---|---|---|
| Subtype/ | | Substrate | Modification | | size | truncation | size | Catalytic |
| Complex | Name | (if known) | (if known) | Organism | (aa) | (aa) | (aa) | domain |
| — | SET/TAF-1β | — | — | M. musculus | 289 | 1-289 | 289 | — |
| | | | | | | (Cervoni) | | |

It is also preferred to target endogenous (regulatory) control elements (such as enhancers and silencers) in addition to a promoter or promoter-proximal elements. Thus, the present disclosure can also be used to target endogenous control elements (including enhancers and silencers) in addition to targeting of the promoter. These control elements can be located upstream and downstream of the transcriptional start site (TSS), starting from 200 bp from the TSS to 100 kb away. Targeting of known control elements can be used to activate or repress the gene of interest. In some cases, a single control element can influence the transcription of multiple target genes. Targeting of a single control element could therefore be used to control the transcription of multiple genes simultaneously.

Targeting of putative control elements on the other hand (e.g. by tiling the region of the putative control element as well as 200 bp up to 100 kB around the element) can be used as a means to verify such elements (by measuring the transcription of the gene of interest) or to detect novel control elements (e.g. by tiling 100 kb upstream and downstream of the TSS of the gene of interest). In addition, targeting of putative control elements can be useful in the context of understanding genetic causes of disease. Many mutations and common SNP variants associated with disease phenotypes are located outside coding regions. Targeting of such regions with either the activation or repression systems described herein can be followed by readout of transcription of either a) a set of putative targets (e.g. a set of genes located in closest proximity to the control element) or b) whole-transcriptome readout by e.g. RNAseq or microarray. This would allow for the identification of likely candidate genes involved in the disease phenotype. Such candidate genes could be useful as novel drug targets.

Histone acetyltransferase (HAT) inhibitors are mentioned herein. However, an alternative in some embodiments is for the one or more functional domains to comprise an acetyltransferase, preferably a histone acetyltransferase. These are useful in the field of epigenomics, for example in methods of interrogating the epigenome. Methods of interrogating the epigenome may include, for example, targeting epigenomic sequences. Targeting epigenomic sequences may include the guide being directed to an epigenomic target sequence. Epigenomic target sequence may include, in some embodiments, include a promoter, silencer or an enhancer sequence.

Use of a functional domain linked to a Cas9 effector protein as described herein, preferably a dead-Cas9 effector protein, more preferably a dead-FnCas9 effector protein, to target epigenomic sequences can be used to activate or repress promoters, silencer or enhancers.

Examples of acetyltransferases are known but may include, in some embodiments, histone acetyltransferases. In some embodiments, the histone acetyltransferase may comprise the catalytic core of the human acetyltransferase p300 (Gerbasch & Reddy, Nature Biotech 6 Apr. 2015).

In some preferred embodiments, the functional domain is linked to a dead-Cas9 effector protein to target and activate epigenomic sequences such as promoters or enhancers. One or more guides directed to such promoters or enhancers may also be provided to direct the binding of the CRISPR enzyme to such promoters or enhancers.

The term “associated with” is used here in relation to the association of the functional domain to the Cas9 effector protein or the adaptor protein. It is used in respect of how one molecule ‘associates’ with respect to another, for example between an adaptor protein and a functional domain, or between the Cas9 effector protein and a functional domain. In the case of such protein-protein interactions, this association may be viewed in terms of recognition in the way an antibody recognizes an epitope. Alternatively, one protein may be associated with another protein via a fusion of the two, for instance one subunit being fused to another subunit. Fusion typically occurs by addition of the amino acid sequence of one to that of the other, for instance via splicing together of the nucleotide sequences that encode each protein or subunit. Alternatively, this may essentially be viewed as binding between two molecules or direct linkage, such as a fusion protein. In any event, the fusion protein may include a linker between the two subunits of interest (i.e. between the enzyme and the functional domain or between the adaptor protein and the functional domain). Thus, in some embodiments, the Cas9 effector protein or adaptor protein is associated with a functional domain by binding thereto. In other embodiments, the Cas9 effector protein or adaptor protein is associated with a functional domain because the two are fused together, optionally via an intermediate linker.

Attachment of a functional domain or fusion protein can be via a linker, e.g., a flexible glycine-serine (GlyGlyGlySer) (SEQ ID NO: 18) or (GGGS)₃ (SEQ ID NO: 19) or a rigid alpha-helical linker such as (Ala(GluAlaAlaAlaLys)Ala) (SEQ ID NO: 20). Linkers such as (GGGGS)₃ (SEQ ID NO: 17) are preferably used herein to separate protein or peptide domains. (GGGGS)₃ (SEQ ID NO: 17) is preferable because it is a relatively long linker (15 amino acids). The glycine residues are the most flexible and the serine residues enhance the chance that the linker is on the outside of the protein. (GGGGS)₆ (SEQ ID NO: 21), (GGGGS)₉ (SEQ ID NO: 22) or (GGGGS)₁₂ (SEQ ID NO: 23) may preferably be used as alternatives. Other preferred alternatives are (GGGGS)₁ (SEQ ID NO: 24), (GGGGS)₂ (SEQ ID NO: 25), (GGGGS)₄ (SEQ ID NO: 26), (GGGGS)₅ (SEQ ID NO: 27), (GGGGS)₇ (SEQ ID NO: 28), (GGGGS)₈ (SEQ ID NO: 29), (GGGGS)₁₀ (SEQ ID NO: 30), or (GGGGS)₁₁ (SEQ ID NO: 31). Alternative linkers are available, but highly flexible linkers are thought to work best to allow for maximum opportunity for the 2 parts of the Cas9 to come together and thus reconstitute Cas9 activity. One alternative is that the NLS of nucleoplasmin can be used as a linker. For example, a linker can also be used between the Cas9 and any functional domain. Again, a (GGGGS)₃ (SEQ ID NO: 17) linker may be used here (or the 6, 9, or 12 repeat versions therefore) or the NLS of nucleoplasmin can be used as a linker between Cas9 and the functional domain.

### Reverse Transcriptase Domain

The one or more functional domains may be one or more reverse transcriptase domains. In some embodiments, the systems comprise an engineered system for modifying a target polynucleotide comprising: a Cas protein or a variant thereof (e.g., dCas); a reverse transcriptase (RT) domain; a RNA template comprising or encoding a donor polynucleotide to be inserted to a target sequence of the target polynucleotide; and a guide molecule.

The reverse transcriptase may generate single-strand DNA based on the RNA template. The single-strand DNA may be generated by a non-retron, retron, or DGR. In some examples, the single-strand DNA may be generated from a self-priming RNA template. A self-priming RNA template may be used to generate a DNA without the need of a separate primer.

A reverse transcriptase domain may be a reverse transcriptase or a fragment thereof. A wide variety of reverse transcriptases (RT) may be used in alternative embodiments of the present invention, including prokaryotic and eukaryotic RT, provided that the RT functions within the host to generate a donor polynucleotide sequence from the RNA template. If desired, the nucleotide sequence of a native RT may be modified, for example using known codon optimization techniques, so that expression within the desired host is optimized. A reverse transcriptase (RT) is an enzyme used to generate complementary DNA (cDNA) from an RNA template, a process termed reverse transcription. Reverse transcriptases are used by retroviruses to replicate their genomes, by retrotransposon mobile genetic elements to proliferate within the host genome, by eukaryotic cells to extend the telomeres at the ends of their linear chromosomes, and by some non-retroviruses such as the hepatitis B virus, a member of the Hepadnaviridae, which are dsDNA-RT viruses. Retroviral RT has three sequential biochemical activities: RNA-dependent DNA polymerase activity, ribonuclease H, and DNA-dependent DNA polymerase activity. Collectively, these activities enable the enzyme to convert single-stranded RNA into double-stranded cDNA. In certain embodiments, the RT domain of a reverse transcriptase is used in the present invention. The domain may include only the RNA-dependent DNA polymerase activity. In some examples, the RT domain is non-mutagenic, i.e., dose not cause mutation in the donor polynucleotide (e.g., during the reverse transcriptase process). In some cases, In some examples, the RT domain may be non-retron RT, e.g., a viral RT or a human endogenous RTs. In some examples, the RT domain may be retron RT or DGRs RT. In some example, the RT may be less mutagenic than a counterpart wildtype RT. In some embodiments, the RT herein is not mutagenic.

### Retrons

In certain embodiments, a donor template for homologous recombination is generated by use of a self-priming RNA template for reverse transcription. A non-limiting example of a self-priming reverse transcription system is the retron system. By the term “retron” it is meant a genetic element which encodes components enabling the synthesis of branched RNA-linked single stranded DNA (msDNA) and a reverse transcriptase. Retrons which encode msDNA are known in the art, for example, but not limited to U.S. Pat. Nos. 6,017,737; 5,849,563; 5,780,269; 5,436,141; 5,405,775; 5,320,958; CA 2,075,515; all of which are herein incorporated by reference).

In certain embodiments, the reverse transcriptase domain is a retron RT domain. In certain embodiments, the RNA template encodes a retron RNA template that is recognized and reverse transcribed by the retron reverse transcriptase domain. Conserved across many bacterial species, retrons are highly efficient reverse transcription systems of relatively unknown function. The retron system consists of the retron RT protein, as well as the msr and msd transcripts, which function as the primer and template sequences respectively. All components of the retron system are expressed from a single open reading frame as a single transcript including the msr-msd and encoding the retron RT protein (Lampson, et al., 2005, Retrons, msDNA, and the bacterial genome. Cytogenet Genome Res 110:491-499). The msr element ORF of a retron provides for the RNA portion of the msDNA molecule, while the msd element ORF provides for the DNA portion of the msDNA molecule. The primary transcript from the msr-msd region is thought to serve as both a template and a primer to produce the msDNA. Synthesis of msDNA is primed from an internal rG residue of the RNA transcript using its 2′-OH group. Modification of msd, or msr may also be made to permit insertion of a RNA template encoding a donor polynucleotide within the msd without altering the functioning of or the production of msDNA. The RNA template encoding a donor polynucleotide sequence may be any length but is preferably less than about 5 kb nucleotides, or also less than about 2 kb, or also less than 500 bases, provided that an msDNA product is produced.

### Diversity Generating Retroelements (DGRs)

In certain embodiments, the one or more functional domains may be a diversity generating retroelement(s) (e.g., DGR described in US20100041033A1). In some embodiments, the DGR may insert a donor polynucleotide with its homing mechanism. For example, the DGR may be associated with a catalytically inactive Cas protein (e.g., a dead Cas), and integrate the single-strand DNA using a homing mechanism. In some examples, the DRG may be less mutagenic than a counterpart wildtype DGR. In some examples, the DGR is not error-prone. In some embodiments, the DGR herein is not mutagenic. The non-mutagenic DGR may be a mutant of a wild type DGR. As used herein, the term “DGR” encompasses both diversity generating retroelement polynucleotides and proteins encoded by diversity generating retroelement polynucleotides. In some examples, DGR may be proteins encoded by diversity generating retroelement polynucleotides and having reverse transcriptase activity. In some examples, DGR may be proteins encoded by diversity generating retroelement polynucleotides, and having reverse transcriptase activity and integrase activity. In some cases, the template or donor polynucleotide may be encoded by a diversity generating retroelement polynucleotide. In certain cases, the template may be a polynucleotide different from the diversity generating retroelement polynucleotide, e.g., provided as a separate construct or molecule.

In some embodiments, the DGR herein also include a Group II intron (and any proteins and polynucleotides encoded), which is mobile ribozymes that self-splice from precursor RNAs to yield excised intron lariat RNAs, which then invade new genomic DNA sites by reverse splicing. Examples of Group II intron include those described in Lambowitz A M et al., Group II Introns: Mobile Ribozymes that Invade DNA, Cold Spring Harb Perspect Biol. 2011 August; 3(8): a003616.

In some embodiments, the diversity-generating retroelements (DGRs) are genetic elements that can produce targeted, massive variations in the genomes that carry these elements. In some embodiments, the DGR systems rely on error-prone reverse transcriptases to produce mutagenized cDNA (containing A-to-N mutations) from a template region (TR), to replace a segment called variable region (VR) that is similar to the TR region—this process is called mutagenic retrohoming (see, e.g., Sharifi and Ye, MyDGR: a server for identification and characterization of diversity-generating retroelements. Nucleic Acids Res. 2019 Jul. 2; 47(W1): W289-W294). DGRs may include a unique family of retroelements that generate sequence diversity of DNA. They exist widely in bacteria, archaea, phage and plasmid, and benefit their hosts by introducing variations and accelerating the evolution of target proteins (see, e.g., Yan et al., Discovery and characterization of the evolution, variation and functions of diversity-generating retroelements using thousands of genomes and metagenomes. BMC Genomics. 2019; 20: 595). The first DGR was discovered in a Bordetella phage, BPP-1. Bordetella causes the respiratory infection in humans and many other mammals, controlled by the BvgAS signal transduction system. The surface of Bordetella is highly variable owing to the dynamic gene expression in the infectious cycle. The invasion of BPP-1 to Bordetella relies on the phage tail fiber protein Mtd. With the process of mutagenic reverse transcription and cDNA integration, DGR may introduce multiple nucleotide substitutions to Mtd gene and generates different receptor-binding molecules, thus making BPP-1 the ability to invade Bordetellae with diverse cell surfaces.

The systems may be used to generate an ssDNA donor using a retron- or DGR RT, which is then integrated by homologous recombination upon target cleavage or nicking using a Cas nuclease. In some embodiments, the systems may comprise DGRs and/or Group-II intron reverse transcriptases. The homing mechanism of DGRs or Group-II introns may be used in modifying a target polynucleotide. The DGRs or Group-II introns reverse transcriptase may be guided to a target polynucleotide by tethering to a nuclease-dead Cas nuclease, TALE, or ZF protein. In another embodiment, a non-retron/DGR reverse transcriptase (e.g. a viral RT) may be used for generating cDNA off of a self-priming RNA. In some embodiments, a ssDNA may be generated by an RT, but integrate it using a dead Cas enzyme, creating an accessible R-loop instead of nicking/cleaving.

### Topoisomerases

The one or more functional domains may be one or more topoisomerase domains. In some embodiments, engineered system for modifying a target polynucleotide comprising: a Cas protein; a topoisomerase domain; and a nucleic acid template comprising or encoding a donor polynucleotide to be inserted to a target sequence of the target polynucleotide. In some examples, two or more of: the Cas protein; topoisomerase domain; and nucleic acid template may form a complex. In some examples, two or more of: the Cas protein; topoisomerase domain, may be comprised in a fusion protein.

Topoisomerases are a class of enzymes that modify the topological state of DNA via the breakage and rejoining of nucleic acid strands. In some cases, a topoisomerase may be a DNA topoisomerase, which is an enzyme that controls and alters the topologic states of DNA during transcription, and catalyzes the transient breaking and rejoining of a single strand of DNA which allows the strands to pass through one another, thus altering the topology of DNA.

In some embodiments, the topoisomerase domain is capable of ligating the donor polynucleotide with the target polynucleotide. The ligation may be achieved by sticky end or blunt end ligation. In an example, the donor polynucleotide may comprise a overhang comprising a sequence complementary to a region of the target polynucleotide. Examples of ligating the donor polynucleotide with the target polynucleotide include those of TOPO cloning, e.g., those described in “The Technology Behind TOPO Cloning,” at www.thermofisher.com/us/en/home/life-science/cloning/topo/topo-resources/the-technology-behind-topo-cloning.html.

In some embodiments, the topoisomerase domain may be associated the donor polynucleotide. For example, the topoisomerase domain is covalently linked to the donor polynucleotide.

In some embodiments, a topoisomerase domain may be provided together with, e.g., associated (e.g., fused) with a Cas protein (e.g., a Cas protein or a variant thereof such as a dead Cas or a Cas nickase). Alternatively or additionally, the topoisomerase domain may be on a molecule different from the Cas protein. In some cases, the topoisomerase domain may be associated with a donor polynucleotide. For example, the topoisomerase domain may be pre-loaded covalently with a donor DNA molecule. Such deign may allow for efficient ligation of only a specific cargo. The topoisomerase domain may ligate the donor polynucleotide (e.g., a DNA molecule) to a target site on a target polynucleotide (e.g., a free double-stranded DNA end). In some embodiments, the donor polynucleotide may have an overhang that comprises a sequence complementary to a region of the target polynucleotide. For example, the overhang may invade into the target polynucleotide at a cut site generated by the Cas protein.

Examples of topoisomerases include type I, including type IA and type IB topoisomerases, which cleave a single strand of a double-stranded nucleic acid molecule, and type II topoisomerases (e.g., gyrases), which cleave both strands of a double-stranded nucleic acid molecule.

Type IA and IB topoisomerases cleave one strand of a double-stranded nucleic acid molecule. In some examples, the cleavage of a double-stranded nucleic acid molecule by type IA topoisomerases generates a 5′ phosphate and a 3′ hydroxyl at the cleavage site, with the type IA topoisomerase covalently binding to the 5′ terminus of a cleaved strand. Cleavage of a double-stranded nucleic acid molecule by type D3 topoisomerases may generate a 3′ phosphate and a 5′ hydroxyl at the cleavage site, with the type IB topoisomerase covalently binding to the 3′ terminus of a cleaved strand.

Examples of Type IA topoisomerases include E. coli topoisomerase I, E. coli topoisomerase III, eukaryotic topoisomerase II, archeal reverse gyrase, yeast topoisomerase III, Drosophila topoisomerase III, human topoisomerase III, Streptococcus pneumoniae topoisomerase III, and the like, including other type IA topoisomerases. A DNA-protein adduct is formed with the enzyme covalently binding to the 5′-thymidine residue, with cleavage occurring between the two thymidine residues.

Examples of Type IB topoisomerases include the nuclear type I topoisomerases present in all eukaryotic cells and those encoded by Vaccinia and other cellular poxviruses. The eukaryotic type IB topoisomerases are exemplified by those expressed in yeast, Drosophila and mammalian cells, including human cells. Viral type IB topoisomerases are exemplified by those produced by the vertebrate poxviruses (Vaccinia, Shope fibroma virus, ORF virus, fowlpox virus, and molluscum contagiosum virus), and the insect poxvirus (Amsacta moorei entomopoxvirus).

Examples of Type II topoisomerases include, bacterial gyrase, bacterial DNA topoisomerase IV, eukaryotic DNA topoisomerase II, and T-even phage encoded DNA topoisomerases. Type II topoisomerases may have both cleaving and ligating activities. Substrate double-stranded nucleic acid molecules of type II topoisomerase can be prepared such that the type II topoisomerase can form a covalent linkage to one strand at a cleavage site. For example, calf thymus type II topoisomerase can cleave a substrate ds nucleic acid molecule containing a 5′ recessed topoisomerase recognition site positioned three nucleotides from the 5′ end, resulting in dissociation of the three nucleic acid molecule 5′ to the cleavage site and covalent binding of the topoisomerase to the 5′ terminus of the ds nucleic acid molecule. Furthermore, upon contacting such a type II topoisomerase-charged ds nucleic acid molecule with a second nucleic acid molecule containing a 3′ hydroxyl group, the type II topoisomerase can ligate the sequences together, and then is released from the recombinant nucleic acid molecule.

In some examples, the topoisomerase is DNA topoisomerase I, e.g., a Vaccinia virus topoisomerase I. The topoisomerase may be pre-loaded with a donor polynucleotide. The Vaccinia virus topoisomerase may need a target comprising a 5′-OH group.

### Phosphatases

The systems herein may further comprise a phosphatase domain. A phosphatase is an enzyme capable of removing a phosphate group from a molecule e.g., a nucleic acid such as DNA. Examples of phosphatases include calf intestinal phosphatase, shrimp alkaline phosphatase, Antarctic phosphatase, and APEX alkaline phosphatase.

In some examples, the 5′-OH group of in the target polynucleotide may be generated by a phosphatase. A topoisomerase compatible with a 5′ phosphate target may be used to generate stable loaded intermediates. In some cases, a Cas nuclease that leaves a 5′ OH after cleaving the target polynucleotide may be used. In some cases, the phosphatase domain may be associated with (e.g., fused to) the Cas protein. The phosphatase domain may be capable of generating a —OH group at a 5′ end of the target polynucleotide. The phosphatase may be delivered separated from other components in the system, e.g., as a separate protein, on a separate vector from other components.

### Polymerases

The systems herein may further comprise a polymerase domain. A polymerase refers to an enzyme that synthesizes chains of nucleic acids. The polymerase may be a DNA polymerase or an RNA polymerase.

In some embodiments, the systems comprise an engineered system for modifying a target polynucleotide comprising: a Cas protein; a DNA polymerase domain; and a DNA template comprising a donor polynucleotide to be inserted to a target sequence of the target polynucleotide. In some examples, two or more of: the Cas protein; DNA polymerase domain; and DNA template may form a complex. In some examples, two or more of: the Cas protein; DNA polymerase domain; are comprised in a fusion protein. For example, the Cas protein and DNA polymerase domain may be comprised in a fusion protein.

In some embodiments, the systems may comprise a Cas enzyme (or variant thereof such as a dCas or Cas nickase) and a DNA polymerase (e.g. phi29, T4, T7 DNA polymerase). The systems may further comprise a single-stranded DNA or double-stranded DNA template. The DNA template may comprise i) a first sequence homologous to a target site of the Cas protein on the target polynucleotide, and/or ii) a second sequence homologous to another region of the target polynucleotide. In some embodiments, the template may be a synthetic single-stranded or PCR-generated DNA molecule, (optionally end-protected by modified nucleotides), or a viral genome (e.g. AAV). In another embodiment, the template is generated using a reverse transcriptase. When the system is delivered in to a cell, an endogenous DNA polymerase in the may be used. Alternatively or additionally, an exogenous DNA polymerase may be expressed in the cell.

The DNA template may be end-protected by one or more modified nucleotides, or comprises a portion of a viral genome. In some embodiment, the DNA template comprises LNA or other modifications (e.g., at the 3′ end). The presence of LNA and/or the modifications may lead to more efficient annealing with the 3′ flap generated by Cas protein cleavage.

In yet another embodiment, PRIME editing is used first to create a longer 3′ region (e.g. 20 nucleotides). Examples of prime editing systems and methods include those described in Anzalone A V et al., Search-and-replace genome editing without double-strand breaks or donor DNA, Nature. 2019 Oct. 21. doi: 10.1038/s41586-019-1711-4, which is incorporated by reference herein in its entirety. In such cases, the system comprises a Cas protein with nickase activity, a reverse transcriptase domain, and a DNA polymerase, and a guide molecule comprising a binding sequence capable of hybridizing to the target polynucleotide and a editing sequence. The generated region may be further extended on a DNA template as described herein. The latter may allow generation of a target-independent sequence, compatible with a generic donor sequence.

The Cas protein is capable of generating a first cleavage of in the target sequence and a second cleavage outside the target sequence on the target polynucleotide. In some variations, a second Cas-mediated cleavage in vicinity to the target site may be made, which may enable more efficient invasion of the extended DNA.

Examples of DNA polymerase include Taq, Tne (exo-), Tma (exo-), Pfu (exo-), Pwo (exo-), Thermoanaerobacter thermohydrosulfuricus DNA polymerase, Thermococcus litoralis DNA polymerase I, E. coli DNA polymerase I, Taq DNA polymerase I, Tth DNA polymerase I, Bacillus stearothermophilus (Bst) DNA polymerase I, E. coli DNA polymerase III, bacteriophage T5 DNA polymerase, bacteriophage M2 DNA polymerase, bacteriophage T4 DNA polymerase, bacteriophage T7 DNA polymerase, bacteriophage phi29 DNA polymerase, bacteriophage PRD1 DNA polymerase, bacteriophage phi15 DNA polymerase, bacteriophage phi21DNA polymerase, bacteriophage PZE DNA polymerase, bacteriophage PZA DNA polymerase, bacteriophage Nf DNA polymerase, bacteriophage M2Y DNA polymerase, bacteriophage B103 DNA polymerase, bacteriophage SF5 DNA polymerase, bacteriophage GA-1 DNA polymerase, bacteriophage Cp-5 DNA polymerase, bacteriophage Cp-7 DNA polymerase, bacteriophage PR4 DNA polymerase, bacteriophage PR5 DNA polymerase, bacteriophage PR722 DNA polymerase and bacteriophage L17 DNA polymerase.

### Ligases

In some embodiments, the compositions and systems may comprise a Cas protein and a ligase associated with the Cas protein. The Cas protein may be recruited to the target sequence by a guide RNA, and generate a break on the target sequence. The guide RNA may further comprise a template sequence with desired mutations or other sequence elements. The template sequence may be ligated to the target sequence to introduce the mutations or other sequence elements to the nucleic acid molecule. The Cas protein may be a nickase that generates a single-strand break on nucleic acid molecule, and the ligase may be a single-strand DNA ligase. In some embodiments, the systems comprise a pair of CRISPR-Cas complexes with two distinct guide sequences. Each CRISPR-Cas complex can target one strand of a double-stranded polynucleotides, and work together to effectively modify the sequence of the double-stranded polynucleotides.

In some examples, the Cas9 is associated with a ligase or functional fragment thereof. The ligase may ligate a single-strand break (a nick) generated by the Cas9. In certain cases, the ligase may ligate a double-strand break generated by the Cas9. In certain examples, the Cas9 is associated with a reverse transcriptase or functional fragment thereof.

The present disclosure further provides systems and methods of modifying a nucleic acid sequence using a pair of distinct CRISPR-Cas complexes, said systems and methods comprising: (a) an engineered Cas protein connected to or complexed with a ligase; (b) two distinct guide RNA sequences complexed with such Cas-ligase protein to form a first and a second distinct CRISPR-Cas complexes; (c) the first CRISPR-Cas complex binding to one strand of a target double-stranded polynucleotide sequence, and the second CRISPR-Cas complex binding to another strand of the target double-stranded polynucleotide sequence; (d) upon binding of the said complexes to the locus of interest the effector protein induces the modification of the sequences associated with or at the target locus of interest, whereby the two CRISPR-Cas complexes work together on different strands of the double-stranded target sequence and modify the sequence.

One of the advantages of using such a “pair” of CRISPR-Cas complexes includes high efficiency in modifying the sequence associated with or at the locus of interest of target double-stranded polynucleotides.

In some embodiments, the Cas protein can be a nickase. In a preferred embodiment, a ligase is linked to the Cas protein. The ligase can ligate the donor sequence to the target sequence. The ligase can be a single-strand DNA ligase or a double-strand DNA ligase. The ligase can be fused to the carboxyl-terminus of a Cas protein, or to the amino-terminus of a Cas protein.

As used herein the term “ligase” refers to an enzyme, which catalyzes the joining of breaks (e.g., double-stranded breaks or single-stranded breaks (“nicks”) between adjacent bases of nucleic acids. For example, a ligase may be an enzyme capable of forming intra- or inter-molecular covalent bonds between a 5′ phosphate group and a 3′ hydroxyl group. The term “ligate” refers to the reaction of covalently joining adjacent oligonucleotides through formation of an internucleotide linkage.

DNA ligases fall into two general categories: ATP-dependent DNA ligases (EC 6.5.1.1), and NAD (+) dependent DNA ligases (EC 6.5.1.2). NAD (+) dependent DNA ligases are found only in bacteria (and some viruses) while ATP-dependent DNA ligases are ubiquitous. The ATP-dependent DNA ligases can be divided into four classes: DNA ligase I, II, III, and IV. DNA ligase I links Okazaki fragments to form a continuous strand of DNA; DNA ligase II is an alternatively spliced form of DNA ligase III, found only in non-dividing cells; DNA ligase III is involved in base excision repair; and DNA ligase IV is involved in the repair of DNA double-strand breaks by non-homologous end joining (NHEJ). Amongst all ligases, there are two types of prokaryotic and one type of eukaryotic ligases that are particularly well suited for facilitating the blunt ended double stranded DNA ligation: Prokaryotic DNA ligases (T3 and T4) and Eukaryotic DNA ligase (Ligase 1).

In some cases, the ligase is specific for double-stranded nucleic acids (e.g., dsDNA, dsRNA, RNA/DNA duplex). An example of a ligase specific for double-stranded DNA and DNA/RNA hybrids is T4 DNA ligase. In some cases, the ligase is specific for single-stranded nucleic acids (e.g., ssDNA, ssRNA). An example of such ligase is CircLigase II. In some cases, the ligase is specific for RNA/DNA duplexes. In some cases, the ligase is able to work on single-stranded, double-stranded, and/or RNA/DNA nucleic acids in any combination.

In some cases, the ligase may be a pan-ligase, which is a single ligase with the ability to ligate both DNA and RNA targets. The ligase may be specific for a target (e.g., DNA-specific or RNA-specific). In some cases, the ligase may be a dual ligase system that include DNA-specific, RNA-specific, and/or pan-ligases, in any combination.

Examples of ligases that can be used with the disclosure include T4 DNA Ligase, T3 DNA Ligase, T7 DNA Ligase, E. coli DNA Ligase, HiFi Taq DNA Ligase, 9° N™ DNA Ligase, Taq DNA Ligase, SplintR® Ligase (also known as. PBCV-1 DNA Ligase or Chlorella virus DNA Ligase), Thermostable 5′ AppDNA/RNA Ligase, T4 RNA Ligase, T4 RNA Ligase 2, T4 RNA Ligase 2 Truncated, T4 RNA Ligase 2 Truncated K227Q, T4 RNA Ligase 2, Truncated K Q, RtcB Ligase (joins single stranded RNA with a 3″-phosphate or 2′,3′-cyclic phosphate to another RNA), CircLigase II, CircLigase ssDNA Ligase, CircLigase RNA Ligase, or Ampligase® Thermostable DNA Ligas, NAD-dependent ligases including Taq DNA ligase, Thermus filiformis DNA ligase, Escherichia coli DNA ligase, Tth DNA ligase, Thermus scotoductus DNA ligase (I and II), thermostable ligase, Ampligase thermostable DNA ligase, VanC-type ligase, 9° N DNA Ligase, Tsp DNA ligase, and novel ligases discovered by bioprospecting; ATP-dependent ligases including T4 RNA ligase, T4 DNA ligase, T3 DNA ligase, T7 DNA ligase, Pfu DNA ligase, DNA ligase I, DNA ligase III, DNA ligase IV, and novel ligases discovered by bioprospecting, and wild-type, mutant isoforms, and genetically engineered variants thereof. In some embodiments, the examples of the ligases include those used in sequencing by synthesis or sequencing by ligation reactions.

### Split Proteins

It is noted that in this context, and more generally for the various applications as described herein, the use of a split version of the Cas protein can be envisaged. Indeed, this may not only allow increased specificity but may also be advantageous for delivery. The Cas is split in the sense that the two parts of the Cas enzyme substantially comprise a functioning Cas. The split may be so that the catalytic domain(s) are unaffected. That Cas may function as a nuclease or it may be a dead-Cas which is essentially an RNA-binding protein with very little or no catalytic activity, due to typically mutation(s) in its catalytic domains.

Each half of the split Cas may be fused to a dimerization partner. By means of example, and without limitation, employing rapamycin sensitive dimerization domains, allows to generate a chemically inducible split Cas for temporal control of Cas activity. Cas can thus be rendered chemically inducible by being split into two fragments and that rapamycin-sensitive dimerization domains may be used for controlled reassembly of the Cas. The two parts of the split Cas can be thought of as the N′ terminal part and the C′ terminal part of the split Cas. The fusion is typically at the split point of the Cas. In other words, the C′ terminal of the N′ terminal part of the split Cas is fused to one of the dimer halves, whilst the N′ terminal of the C′ terminal part is fused to the other dimer half.

The Cas does not have to be split in the sense that the break is newly created. The split point is typically designed in silico and cloned into the constructs. Together, the two parts of the split Cas, the N′ terminal and C′ terminal parts, form a full Cas, comprising preferably at least 70% or more of the wildtype amino acids (or nucleotides encoding them), preferably at least 80% or more, preferably at least 90% or more, preferably at least 95% or more, and most preferably at least 99% or more of the wildtype amino acids (or nucleotides encoding them). Some trimming may be possible, and mutants are envisaged. Non-functional domains may be removed entirely. What is important is that the two parts may be brought together and that the desired Cas function is restored or reconstituted. The dimer may be a homodimer or a heterodimer.

In certain embodiments, the Cas effector as described herein may be used for mutation-specific, or allele-specific targeting, such as. for mutation-specific, or allele-specific knockdown.

The effector protein can moreover be fused to another functional RNase domain, such as a non-specific RNase or Argonaute 2, which acts in synergy to increase the RNase activity or to ensure further degradation of the message.

### Modulating Cas Proteins

The present disclosure provides accessory proteins that modulate CRISPR protein function. In certain embodiments, the accessory protein modulates catalytic activity of a CRISPR protein. In an embodiment of the present disclosure an accessory protein modulates targeted, or sequence specific, nuclease activity. In an embodiment of the present disclosure, an accessory protein modulates collateral nuclease activity. In an embodiment of the present disclosure, an accessory protein modulates binding to a target nucleic acid.

According to the present disclosure, the nuclease activity to be modulated can be directed against nucleic acids comprising or consisting of RNA, including without limitation mRNA, miRNA, siRNA and nucleic acids comprising cleavable RNA linkages along with nucleotide analogs. In an embodiment of the present disclosure, the nuclease activity to be modulated can be directed against nucleic acids comprising or consisting of DNA, including without limitation nucleic acids comprising cleavable DNA linkages and nucleic acid analogs.

In an embodiment of the present disclosure, an accessory protein enhances an activity of a CRISPR protein. In certain embodiments, the accessory protein inhibits an activity of a CRISPR protein.

According to the present disclosure, naturally occurring accessory proteins of Type II CRISPR systems comprise small proteins encoded at or near a CRISPR locus that function to modify an activity of a CRISPR protein. In general, a CRISPR locus can be identified as comprising a putative CRISPR array and/or encoding a putative CRISPR effector protein. In an embodiment, an effector protein can be from 800 to 2000 amino acids, or from 900 to 1800 amino acids, or from 950 to 1300 amino acids. In an embodiment, an accessory protein can be encoded within 25 kb, or within 20 kb or within 15 kb, or within 10 kb of a putative CRISPR effector protein or array, or from 2 kb to 10 kb from a putative CRISPR effector protein or array.

In an embodiment of the present disclosure, an accessory protein is from 50 to 300 amino acids, or from 100 to 300 amino acids or from 150 to 250 amino acids or about 200 amino acids.

Identification and use of a CRISPR accessory protein of the present disclosure is independent of CRISPR effector protein classification. Accessory proteins of the present disclosure can be found in association with or engineered to function with a variety of CRISPR effector proteins. Examples of accessory proteins identified and used herein are representative of CRISPR effector proteins generally. It is understood that CRISPR effector protein classification may involve homology, feature location, nucleic acid target (e.g. DNA or RNA), absence or presence of tracr RNA, location of guide/spacer sequence 5′ or 3′ of a direct repeat, or other criteria. In embodiments of the present disclosure, accessory protein identification and use transcend such classifications.

According to the present disclosure, in certain embodiments, enhancing activity of a Type II Cas protein or complex thereof comprises contacting the Type II Cas protein or complex thereof with an accessory protein from the same organism that activates the Cas protein. In other embodiments, enhancing activity of a Type II Cas protein of complex thereof comprises contacting the Type II Cas protein or complex thereof with an activator accessory protein from a different organism within the same subclass (e.g., Type II). In other embodiments, enhancing activity of a Type II Cas protein or complex thereof comprises contacting the Type II Cas protein or complex thereof with an accessory protein not within the subclass (e.g., a Type II Cas protein other than Type II-b with a Type II accessory protein or vice-versa).

According to the present disclosure, in certain embodiments, repressing activity of a Type II Cas protein or complex thereof comprises contacting the Type II Cas protein or complex thereof with an accessory protein from the same organism that represses the Cas protein. In other embodiments, repressing activity of a Type II Cas protein or complex thereof comprises contacting the Type II Cas protein or complex thereof with a repressor accessory protein from a different organism within the same subclass (e.g., Type II-B or Type II-C). In other embodiments, repressing activity of a Type II Cas protein or complex thereof comprises contacting the Type II Cas protein or complex thereof with a repressor accessory protein not within the subclass (e.g., a Type II Cas protein other than Type II-B with a Type II-B repressor accessory protein or vice-versa).

In certain embodiments where the Type II Cas protein and the Type II accessory protein are from the same organism, the two proteins will function together in an engineered CRISPR system. In certain embodiments, it will be desirable to alter the function of the engineered CRISPR system, for example by modifying either or both of the proteins or their expression. In embodiments where the Type II Cas protein and the Type II accessory protein are from different organisms which may be within the same class or different classes, the proteins may function together in an engineered CRISPR system but it will often be desired or necessary to modify either or both of the proteins to function together.

Accordingly, in certain embodiments of the present disclosure either or both of a Cas protein and an accessory protein may be modified to adjust aspects of protein-protein interactions between the Cas protein and accessory protein. In certain embodiments, either or both of a Cas protein and an accessory protein may be modified to adjust aspects of protein-nucleic acid interactions. Ways to adjust protein-protein interactions and protein-nucleic acid interaction include without limitation, fitting molecular surfaces, polar interactions, hydrogen bonds, and modulating van der Waals interactions. In certain embodiments, adjusting protein-protein interactions or protein-nucleic acid binding comprises increasing or decreasing binding interactions. In certain embodiments, adjusting protein-protein interactions or protein-nucleic acid binding comprises modifications that favor or disfavor a conformation of the protein or nucleic acid.

By “fitting”, is meant determining including by automatic, or semi-automatic means, interactions between one or more atoms of a Cas protein (and optionally at least one atoms of a Cas accessory protein), or between one or more atoms of a Cas protein and one or more atoms of a nucleic acid, (or optionally between one or more atoms of a Cas accessory protein and a nucleic acid), and calculating the extent to which such interactions are stable. Interactions include attraction and repulsion, brought about by charge, steric considerations and the like.

The three-dimensional structure of Type II CRISPR protein or complex thereof (and/or a Type II CRISPR accessory protein or complex thereof in the context of Casb) provides in the context of the instant present disclosure an additional tool for identifying additional mutations in orthologs of Cas. The crystal structure can also be basis for the design of new and specific Cass (and optionally Cas accessory proteins). Various computer-based methods for fitting are described further. Binding interactions of Cass (and optionally accessory proteins), and nucleic acids can be examined through the use of computer modeling using a docking program. Docking programs are known; for example, GRAM, DOCK or AUTODOCK (see Walters et al. Drug Discovery Today, vol. 3, no. 4 (1998), 160-178, and Dunbrack et al. Folding and Design 2 (1997), 27-42). This procedure can include computer fitting to ascertain how well the shape and the chemical structure of the binding partners. Computer-assisted, manual examination of the active site or binding site of a Type II system may be performed. Programs such as GRID (P. Goodford, J. Med. Chem, 1985, 28, 849-57)—a program that determines probable interaction sites between molecules with various functional groups—may also be used to analyze the active site or binding site to predict partial structures of binding compounds. Computer programs can be employed to estimate the attraction, repulsion or steric hindrance of the two binding partners, e.g., components of a Type II CRISPR system, or a nucleic acid molecule and a component of a Type II CRISPR system.

Amino acid substitutions may be made on the basis of differences or similarities in amino acid properties (such as polarity, charge, solubility, hydrophobicity, hydrophilicity, and/or the amphipathic nature of the residues) and it is therefore useful to group amino acids together in functional groups. Amino acids may be grouped together based on the properties of their side chains alone. In comparing orthologs, there are likely to be residues conserved for structural or catalytic reasons. These sets may be described in the form of a Venn diagram (Livingstone C. D. and Barton G. J. (1993) “Protein sequence alignments: a strategy for the hierarchical analysis of residue conservation” Comput. Appl. Biosci. 9: 745-756) (Taylor W. R. (1986) “The classification of amino acid conservation” J. Theor. Biol. 119; 205-218). Conservative substitutions may be made, for example according to the table below which describes a generally accepted Venn diagram grouping of amino acids (see Table 5 below).

TABLE 6

| Set | | Sub-set | |
|---|---|---|---|
| Hydrophobic | F W Y H K M | Aromatic | F W Y H |
| | I L V A G C | | |
| | | Aliphatic | I L V |
| Polar | W Y H K R E | Charged | H K R E D |
| | D C S T N Q | | |
| | | Positively | H K R |
| | | charged | |
| | | Negatively | E D |
| | | charged | |
| Small | V C A G S P | Tiny | A G S |
| | T N D | | |

In some embodiments, the modifications in Cas may comprise modification of one or more amino acid residues of the Cas protein. In some embodiments, the modifications in Cas may comprise modification of one or more amino acid residues located in a region which comprises residues which are positively charged in the unmodified Cas protein (and/or Cas accessory protein). In some embodiments, the modifications in Cas may comprise modification of one or more amino acid residues which are positively charged in the unmodified Cas protein (and/or Cas accessory protein). In some embodiments, the modifications in Cas may comprise modification of one or more amino acid residues which are not positively charged in the unmodified Cas protein (and/or Cas accessory protein). The modification may comprise modification of one or more amino acid residues which are uncharged in the unmodified Cas protein (and/or Cas accessory protein). The modification may comprise modification of one or more amino acid residues which are negatively charged in the unmodified Cas protein (and/or Cas accessory protein). The modification may comprise modification of one or more amino acid residues which are hydrophobic in the unmodified Cas protein (and/or Cas accessory protein). The modification may comprise modification of one or more amino acid residues which are polar in the unmodified Cas protein (and/or Cas accessory protein). The modification may comprise substitution of a hydrophobic amino acid or polar amino acid with a charged amino acid, which can be a negatively charged or positively charged amino acid. The modification may comprise substitution of a negatively charged amino acid with a positively charged or polar or hydrophobic amino acid. The modification may comprise substitution of a positively charged amino acid with a negatively charged or polar or hydrophobic amino acid.

Embodiments herein also include sequences (both polynucleotide or polypeptide) which may comprise homologous substitution (substitution and replacement are both used herein to mean the interchange of an existing amino acid residue or nucleotide, with an alternative residue or nucleotide) that may occur i.e., like-for-like substitution in the case of amino acids such as basic for basic, acidic for acidic, polar for polar, etc. Non-homologous substitution may also occur i.e., from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine. Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, which involves the presence of one or more amino acid residues in peptoid form, may be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

Homology modelling: Corresponding residues in other Cas orthologs can be identified by the methods of Zhang et al., 2012 (Nature; 490(7421): 556-60) and Chen et al., 2015 (PLoS Comput Biol; 11(5): e1004248)—a computational protein-protein interaction (PPI) method to predict interactions mediated by domain-motif interfaces. PrePPI (Predicting PPI), a structure based PPI prediction method, combines structural evidence with non-structural evidence using a Bayesian statistical framework. The method involves taking a pair of query proteins and using structural alignment to identify structural representatives that correspond to either their experimentally determined structures or homology models. Structural alignment is further used to identify both close and remote structural neighbors by considering global and local geometric relationships. Whenever two neighbors of the structural representatives form a complex reported in the Protein Data Bank, this defines a template for modelling the interaction between the two query proteins. Models of a complex are created by superimposing the representative structures on their corresponding structural neighbor in the template. This approach is in Dey et al., 2013 (Prot Sci; 22: 359-66).

### Cas Protein Complex System Promotes Non-Homologous End-Joining

In certain embodiments, nuclease-induced non-homologous end-joining (NHEJ) can be used to target gene-specific knockouts. Nuclease-induced NHEJ can also be used to remove (e.g., delete) sequence in a gene of interest. Generally, NHEJ repairs a double-strand break in the DNA by joining together the two ends; however, generally, the original sequence is restored only if two compatible ends, exactly as they were formed by the double-strand break, are perfectly ligated. The DNA ends of the double-strand break are frequently the subject of enzymatic processing, resulting in the addition or removal of nucleotides, at one or both strands, prior to rejoining of the ends. This results in the presence of insertion and/or deletion (indel) mutations in the DNA sequence at the site of the NHEJ repair. Two-thirds of these mutations typically alter the reading frame and, therefore, produce a non-functional protein. Additionally, mutations that maintain the reading frame, but which insert or delete a significant amount of sequence, can destroy functionality of the protein. This is locus dependent as mutations in critical functional domains are likely less tolerable than mutations in non-critical regions of the protein. The indel mutations generated by NHEJ are unpredictable in nature; however, at a given break site certain indel sequences are favored and are over represented in the population, likely due to small regions of microhomology. The lengths of deletions can vary widely; most commonly in the 1-50 bp range, but they can easily be greater than 50 bp, e.g., they can easily reach greater than about 100-200 bp. Insertions tend to be shorter and often include short duplications of the sequence immediately surrounding the break site. However, it is possible to obtain large insertions, and in these cases, the inserted sequence has often been traced to other regions of the genome or to plasmid DNA present in the cells.

Because NHEJ is a mutagenic process, it may also be used to delete small sequence motifs as long as the generation of a specific final sequence is not required. If a double-strand break is targeted near to a short target sequence, the deletion mutations caused by the NHEJ repair often span, and therefore remove, the unwanted nucleotides. For the deletion of larger DNA segments, introducing two double-strand breaks, one on each side of the sequence, can result in NHEJ between the ends with removal of the entire intervening sequence. Both of these approaches can be used to delete specific DNA sequences; however, the error-prone nature of NHEJ may still produce indel mutations at the site of repair.

Both double strand cleaving Cas9 molecules and single strand, or nickase, Cas9 molecules can be used in the methods and compositions described herein to generate NHEJ-mediated indels. NHEJ-mediated indels targeted to the gene, e.g., a coding region, e.g., an early coding region of a gene of interest can be used to knockout (i.e., eliminate expression of) a gene of interest. For example, early coding region of a gene of interest includes sequence immediately following a transcription start site, within a first exon of the coding sequence, or within 500 bp of the transcription start site (e.g., less than 500, 450, 400, 350, 300, 250, 200, 150, 100 or 50 bp).

In an embodiment, in which a guide RNA and Cas9 nuclease generate a double strand break for the purpose of inducing NHEJ-mediated indels, a guide RNA may be configured to position one double-strand break in close proximity to a nucleotide of the target position. In an embodiment, the cleavage site may be between 0-500 bp away from the target position (e.g., less than 500, 400, 300, 200, 100, 50, 40, 30, 25, 20, 15, 10, 9, 8, 7, 6, 5, 4, 3, 2 or 1 bp from the target position).

In an embodiment, in which two guide RNAs complexing with Cas9 nickases induce two single strand breaks for the purpose of inducing NHEJ-mediated indels, two guide RNAs may be configured to position two single-strand breaks to provide for NHEJ repair a nucleotide of the target position.

### Guide Sequences

The systems and compositions herein may further comprise one or more guide sequences. The guide sequences may hybridize or be capable of hybridizing with a target sequence. In embodiments of the present disclosure the terms guide sequence and guide RNA and crRNA are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. Preferably the guide sequence is 10-30 nucleotides long, such as 30 nucleotides long. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art. A guide sequence may be selected to target any target sequence. In some embodiments, the target sequence is a sequence within a genome of a cell. Exemplary target sequences include those that are unique in the target genome.

In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Vectors for and that result in expression in a eukaryotic cell can be referred to herein as “eukaryotic expression vectors.” Common expression vectors of utility in recombinant DNA techniques are often in the form of plasmids.

Recombinant expression vectors can comprise a nucleic acid of the present disclosure in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell).

The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

Advantageous vectors include lentiviruses and adeno-associated viruses, and types of such vectors can also be selected for targeting particular types of cells.

As used herein, the term “crRNA” or “guide RNA” or “single guide RNA” or “sgRNA” or “one or more nucleic acid components” of a Type II CRISPR-Cas locus effector protein comprises any polynucleotide sequence having sufficient complementarity with a target nucleic acid sequence to hybridize with the target nucleic acid sequence and direct sequence-specific binding of a nucleic acid-targeting complex to the target nucleic acid sequence.

In certain embodiments, the CRISPR system as provided herein can make use of a crRNA or analogous polynucleotide comprising a guide sequence, wherein the polynucleotide is an RNA, a DNA or a mixture of RNA and DNA, and/or wherein the polynucleotide comprises one or more nucleotide analogs. The sequence can comprise any structure, including but not limited to a structure of a native crRNA, such as a bulge, a hairpin or a stem loop structure. In certain embodiments, the polynucleotide comprising the guide sequence forms a duplex with a second polynucleotide sequence which can be an RNA or a DNA sequence.

In certain embodiments, guides of the present disclosure comprise non-naturally occurring nucleic acids and/or non-naturally occurring nucleotides and/or nucleotide analogs, and/or chemically modifications. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the present disclosure, a guide nucleic acid comprises ribonucleotides and non-ribonucleotides. In one such embodiment, a guide comprises one or more ribonucleotides and one or more deoxyribonucleotides. In an embodiment of the present disclosure, the guide comprises one or more non-naturally occurring nucleotide or nucleotide analog such as a nucleotide with phosphorothioate linkage, boranophosphate linkage, a locked nucleic acid (LNA) nucleotides comprising a methylene bridge between the 2′ and 4′ carbons of the ribose ring, or bridged nucleic acids (BNA). Other examples of modified nucleotides include 2′-O-methyl analogs, 2′-deoxy analogs, 2-thiouridine analogs, N6-methyladenosine analogs, or 2′-fluoro analogs. Further examples of modified bases include, but are not limited to, 2-aminopurine, 5-bromo-uridine, pseudouridine (Ψ), N1-methylpseudouridine (me1Ψ), 5-methoxyuridine(5moU), inosine, 7-methylguanosine. Examples of guide RNA chemical modifications include, without limitation, incorporation of 2′-O-methyl (M), 2′-O-methyl 3′phosphorothioate (MS), S-constrained ethyl (cEt), or 2′-O-methyl 3′thioPACE (MSP) at one or more terminal nucleotides. Such chemically modified guide RNAs can comprise increased stability and increased activity as compared to unmodified guide RNAs, though on-target vs. off-target specificity is not predictable. (See, Hendel, 2015, Nat Biotechnol. 33(9):985-9, doi: 10.1038/nbt.3290, published online 29 Jun. 2015; Allerson et al., J. Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Li et al., Nature Biomedical Engineering, 2017, 1, 0066 DOI:10.1038/s41551-017-0066).

In some embodiments, the 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In certain embodiments, a guide comprises ribonucleotides in a region that binds to a target DNA and one or more deoxyribonucleotides and/or nucleotide analogs in a region that binds to Cas9, Cpf1, or C2c1. In an embodiment of the present disclosure, deoxyribonucleotides and/or nucleotide analogs are incorporated in engineered guide structures, such as, without limitation, 5′ and/or 3′ end, stem-loop regions, and the seed region. In certain embodiments, the modification is not in the 5′-handle of the stem-loop regions. Chemical modification in the 5′-handle of the stem-loop region of a guide may abolish its function (see Li, et al., Nature Biomedical Engineering, 2017, 1:0066). In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either the 3′ or the 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at the 5′ and/or the 3′ end of the guide are chemically modified with 2′-O-methyl (M), 2′-O-methyl-3′-phosphorothioate (MS), S-constrained ethyl(cEt), or 2′-O-methyl-3′-thioPACE (MSP). Such modification can enhance genome editing efficiency (see Hendel et al., Nat. Biotechnol. (2015) 33(9): 985-989). In certain embodiments, all of the phosphodiester bonds of a guide are substituted with phosphorothioates (PS) for enhancing levels of gene disruption. In certain embodiments, more than five nucleotides at the 5′ and/or the 3′ end of the guide are chemically modified with 2′-O-Me, 2′-F or S-constrained ethyl(cEt). Such chemically modified guide can mediate enhanced levels of gene disruption (see Ragdarm et al., 0215, PNAS, E7110-E7111). In an embodiment of the present disclosure, a guide is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells generically edited by a CRISPR system (see Lee et al., eLife, 2017, 6:e25312, DOI:10.7554)

In some embodiments, the modification to the guide is a chemical modification, an insertion, a deletion or a split. In some embodiments, the chemical modification includes, but is not limited to, incorporation of 2′-O-methyl (M) analogs, 2′-deoxy analogs, 2-thiouridine analogs, N6-methyladenosine analogs, 2′-fluoro analogs, 2-aminopurine, 5-bromo-uridine, pseudouridine (Ψ), N1-methylpseudouridine (me1Ψ), 5-methoxyuridine(5moU), inosine, 7-methylguanosine, 2′-O-methyl-3′-phosphorothioate (MS), S-constrained ethyl(cEt), phosphorothioate (PS), or 2′-O-methyl-3′-thioPACE (MSP). In some embodiments, the guide comprises one or more of phosphorothioate modifications. In certain embodiments, at least 1, 2, 3, 4, 5, 6, 7, 8, 9, 10, 11, 12, 13, 14, 15, 16, 17, 18, 19, 20, or 25 nucleotides of the guide are chemically modified. In certain embodiments, one or more nucleotides in the seed region are chemically modified. In certain embodiments, one or more nucleotides in the 3′-terminus are chemically modified. In certain embodiments, none of the nucleotides in the 5′-handle is chemically modified. In some embodiments, the chemical modification in the seed region is a minor modification, such as incorporation of a 2′-fluoro analog. In a specific embodiment, one nucleotide of the seed region is replaced with a 2′-fluoro analog. In some embodiments, 5 or 10 nucleotides in the 3′-terminus are chemically modified. Such chemical modifications at the 3′-terminus of the Cpf1 CrRNA improve gene cutting efficiency (see Li, et al., Nature Biomedical Engineering, 2017, 1:0066). In a specific embodiment, 5 nucleotides in the 3′-terminus are replaced with 2′-fluoro analogues. In a specific embodiment, 10 nucleotides in the 3′-terminus are replaced with 2′-fluoro analogues. In a specific embodiment, 5 nucleotides in the 3′-terminus are replaced with 2′-O-methyl (M) analogs.

In some embodiments, the loop of the 5′-handle of the guide is modified. In some embodiments, the loop of the 5′-handle of the guide is modified to have a deletion, an insertion, a split, or chemical modifications. In certain embodiments, the loop comprises 3, 4, or 5 nucleotides. In certain embodiments, the loop comprises the sequence of UCUU, UUUU, UAUU, or UGUU.

In one aspect, the guide comprises portions that are chemically linked or conjugated via a non-phosphodiester bond. In one aspect, the guide comprises, in non-limiting examples, direct repeat sequence portion and a targeting sequence portion that are chemically linked or conjugated via a non-nucleotide loop. In some embodiments, the portions are joined via a non-phosphodiester covalent linker. Examples of the covalent linker include but are not limited to a chemical moiety selected from the group consisting of carbamates, ethers, esters, amides, imines, amidines, aminotrizines, hydrozone, disulfides, thioethers, thioesters, phosphorothioates, phosphorodithioates, sulfonamides, sulfonates, sulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, portions of the guide are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some embodiments, the non-targeting guide portions can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sulfonyl, ally, propargyl, diene, alkyne, and azide. Once a non-targeting portions of a guide is functionalized, a covalent chemical bond or linkage can be formed between the two oligonucleotides. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, sulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, one or more portions of a guide can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

In some embodiments, the guide portions can be covalently linked using various bioconjugation reactions, loops, bridges, and non-nucleotide links via modifications of sugar, internucleotide phosphodiester bonds, purine and pyrimidine residues. Sletten et al., Angew. Chem. Int. Ed. (2009) 48:6974-6998; Manoharan, M. Curr. Opin. Chem. Biol. (2004) 8: 570-9; Behlke et al., Oligonucleotides (2008) 18: 305-19; Watts, et al., Drug. Discov. Today (2008) 13: 842-55; Shukla, et al., ChemMedChem (2010) 5: 328-49.

In some embodiments, the guide portions can be covalently linked using click chemistry. In some embodiments, guide portions can be covalently linked using a triazole linker. In some embodiments, guide portions can be covalently linked using Huisgen 1,3-dipolar cycloaddition reaction involving an alkyne and azide to yield a highly stable triazole linker (He et al., ChemBioChem (2015) 17: 1809-1812; WO 2016/186745). In some embodiments, guide portions are covalently linked by ligating a 5′-hexyne portion and a 3′-azide portion. In some embodiments, either or both of the 5′-hexyne guide portion and a 3′-azide guide portion can be protected with 2′-acetoxyethl orthoester (2′-ACE) group, which can be subsequently removed using Dharmacon protocol (Scaringe et al., J. Am. Chem. Soc. (1998) 120: 11820-11821; Scaringe, Methods Enzymol. (2000) 317: 3-18).

In some embodiments, guide portions can be covalently linked via a linker (e.g., a non-nucleotide loop) that comprises a moiety such as spacers, attachments, bioconjugates, chromophores, reporter groups, dye labeled RNAs, and non-naturally occurring nucleotide analogues. More specifically, suitable spacers for purposes of this present disclosure include, but are not limited to, polyethers (e.g., polyethylene glycols, polyalcohols, polypropylene glycol or mixtures of ethylene and propylene glycols), polyamines group (e.g., spennine, spermidine and polymeric derivatives thereof), polyesters (e.g., poly(ethyl acrylate)), polyphosphodiesters, alkylenes, and combinations thereof. Suitable attachments include any moiety that can be added to the linker to add additional properties to the linker, such as but not limited to, fluorescent labels. Suitable bioconjugates include, but are not limited to, peptides, glycosides, lipids, cholesterol, phospholipids, diacyl glycerols and dialkyl glycerols, fatty acids, hydrocarbons, enzyme substrates, steroids, biotin, digoxigenin, carbohydrates, polysaccharides. Suitable chromophores, reporter groups, and dye-labeled RNAs include, but are not limited to, fluorescent dyes such as fluorescein and rhodamine, chemiluminescent, electrochemiluminescent, and bioluminescent marker compounds. The design of example linkers conjugating two RNA components are also described in WO 2004/015075.

The linker (e.g., a non-nucleotide loop) can be of any length. In some embodiments, the linker has a length equivalent to about 0-16 nucleotides. In some embodiments, the linker has a length equivalent to about 0-8 nucleotides. In some embodiments, the linker has a length equivalent to about 0-4 nucleotides. In some embodiments, the linker has a length equivalent to about 2 nucleotides. Example linker design is also described in WO2011/008730.

In some embodiments, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). The ability of a guide sequence (within a guide RNA or crRNA) to direct sequence-specific binding of a nucleic acid-targeting complex to a target nucleic acid sequence may be assessed by any suitable assay. For example, the components of a CRISPR-Cas system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible, and will occur to those skilled in the art. A guide sequence, and hence a guide RNA or crRNA may be selected to target any target nucleic acid sequence. The target sequence may be DNA. The target sequence may be any RNA sequence. In some embodiments, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within a RNA molecule selected from the group consisting of mRNA, pre-mRNA, and rRNA. In some preferred embodiments, the target sequence may be a sequence within a RNA molecule selected from the group consisting of ncRNA, and lncRNA. In some more preferred embodiments, the target sequence may be a sequence within an mRNA molecule or a pre-mRNA molecule.

In some embodiments, a guide RNA or crRNA is selected to reduce the degree secondary structure within the guide RNA or crRNA. In some embodiments, about or less than about 75%, 50%, 40%, 30%, 25%, 20%, 15%, 10%, 5%, 1%, or fewer of the nucleotides of the guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In some embodiments, a nucleic acid-targeting guide is designed or selected to modulate intermolecular interactions among guide molecules, such as among stem-loop regions of different guide molecules. It will be appreciated that nucleotides within a guide that base-pair to form a stem-loop are also capable of base-pairing to form an intermolecular duplex with a second guide and that such an intermolecular duplex would not have a secondary structure compatible with CRISPR complex formation. Accordingly, is useful to select or design DR sequences in order to modulate stem-loop formation and CRISPR complex formation. In some embodiments, about or less than about 75%, 50%, 40%, 30%, 25%, 20%, 15%, 10%, 5%, 1%, or fewer of nucleic acid-targeting guides are in intermolecular duplexes. It will be appreciated that stem-loop variation will often be within limits imposed by DR-CRISPR effector interactions. One way to modulate stem-loop formation or change the equilibrium between stem-loop and intermolecular duplex is to vary nucleotide pairs in the stem of the stem-loop of a DR. For example, in one embodiment, a G-C pair is replaced by an A-U or U-A pair. In another embodiment, an A-U pair is substituted for a G-C or a C-G pair. In another embodiment, a naturally occurring nucleotide is replaced by a nucleotide analog. Another way to modulate stem-loop formation or change the equilibrium between stem-loop and intermolecular duplex is to modify the loop of the stem-loop of a DR. Without be bound by theory, the loop can be viewed as an intervening sequence flanked by two sequences that are complementary to each other. When that intervening sequence is not self-complementary, its effect will be to destabilize intermolecular duplex formation. The same principle applies when guides are multiplexed: while the targeting sequences may differ, it may be advantageous to modify the stem-loop region in the DRs of the different guides. Moreover, when guides are multiplexed, the relative activities of the different guides can be modulated by balancing the activity of each individual guide. In certain embodiments, the equilibrium between intermolecular stem-loops vs. intermolecular duplexes is determined. The determination may be made by physical or biochemical means and can be in the presence or absence of a CRISPR effector.

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence. In other embodiments, multiple DRs (such as dual DRs) may be present.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In general, degree of complementarity is with reference to the optimal alignment of the sca sequence and tracr sequence, along the length of the shorter of the two sequences. Optimal alignment may be determined by any suitable alignment algorithm, and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher. In certain embodiments, the tracrRNA may not be required. Indeed, the CRISPR-Cas protein from Bergeyella zoohelcum and orthologs thereof do not require a tracrRNA to ensure cleavage of an RNA target.

In further detail, the assay is as follows for a RNA target, provided that a PAM sequence is required to direct recognition. Two E. coli strains are used in this assay. One carries a plasmid that encodes the endogenous effector protein locus from the bacterial strain. The other strain carries an empty plasmid (e.g. pACYC184, control strain). All possible 7 or 8 bp PAM sequences are presented on an antibiotic resistance plasmid (pUC19 with ampicillin resistance gene). The PAM is located next to the sequence of proto-spacer 1 (the RNA target to the first spacer in the endogenous effector protein locus). Two PAM libraries were cloned. One has a 8 random bp 5′ of the proto-spacer (e.g. total of 65536 different PAM sequences=complexity). The other library has 7 random bp 3′ of the proto-spacer (e.g. total complexity is 16384 different PAMs). Both libraries were cloned to have in average 500 plasmids per possible PAM. Test strain and control strain were transformed with 5′PAM and 3′PAM library in separate transformations and transformed cells were plated separately on ampicillin plates. Recognition and subsequent cutting/interference with the plasmid renders a cell vulnerable to ampicillin and prevents growth. Approximately 12 h after transformation, all colonies formed by the test and control strains where harvested and plasmid RNA was isolated. Plasmid RNA was used as template for PCR amplification and subsequent deep sequencing. Representation of all PAMs in the untransformed libraries showed the expected representation of PAMs in transformed cells. Representation of all PAMs found in control strains showed the actual representation. Representation of all PAMs in test strain showed which PAMs are not recognized by the enzyme and comparison to the control strain allows extracting the sequence of the depleted PAM. In particular embodiments, the cleavage, such as the RNA cleavage is not PAM dependent.

For minimization of toxicity and off-target effect, it will be important to control the concentration of guide RNA delivered. Optimal concentrations of nucleic acid—targeting guide RNA can be determined by testing different concentrations in a cellular or non-human eukaryote animal model and using deep sequencing the analyze the extent of modification at potential off-target genomic loci. The concentration that gives the highest level of on-target modification while minimizing the level of off-target modification should be chosen for in vivo delivery. The system is derived advantageously from a CRISPR-Cas system. Dead guide sequences

In one aspect, the present disclosure provides guide sequences which are modified in a manner which allows for formation of the CRISPR Cas complex and successful binding to the target, while at the same time, not either allowing for or not allowing for successful nuclease activity (i.e. without nuclease activity/without indel activity). For matters of explanation such modified guide sequences are referred to as “dead guides” or “dead guide sequences”. These dead guides or dead guide sequences can be thought of as catalytically inactive or conformationally inactive with regard to nuclease activity. Indeed, dead guide sequences may not sufficiently engage in productive base pairing with respect to the ability to promote catalytic activity or to distinguish on-target and off-target binding activity. Briefly, the assay involves synthesizing a CRISPR target RNA and guide RNAs comprising mismatches with the target RNA, combining these with the enzyme and analyzing cleavage based on gels based on the presence of bands generated by cleavage products, and quantifying cleavage based upon relative band intensities.

Hence, in a related aspect, the present disclosure provides a non-naturally occurring or engineered composition CRISPR-Cas system comprising a functional enzyme as described herein, and guide RNA (gRNA) or crRNA wherein the gRNA or crRNA comprises a dead guide sequence whereby the gRNA is capable of hybridizing to a target sequence such that the CRISPR-Cas system is directed to a genomic locus of interest in a cell without detectable RNA cleavage activity of a non-mutant enzyme of the system. It is to be understood that any of the gRNAs or crRNAs according to the present disclosure as described herein elsewhere may be used as dead gRNAs/crRNAs comprising a dead guide sequence.

The ability of a dead guide sequence to direct sequence-specific binding of a CRISPR complex to an RNA target sequence may be assessed by any suitable assay. For example, the components of a CRISPR-Cas system sufficient to form a CRISPR-Cas complex, including the dead guide sequence to be tested, may be provided to a host cell having the corresponding target sequence, such as by transfection with vectors encoding the components of the system, followed by an assessment of preferential cleavage within the target sequence.

As explained further herein, several structural parameters allow for a proper framework to arrive at such dead guides. Dead guide sequences can be typically shorter than respective guide sequences which result in active RNA cleavage. In particular embodiments, dead guides are 5%, 10%, 20%, 30%, 40%, 50%, shorter than respective guides directed to the same.

As explained below and known in the art, one aspect of gRNA or crRNA-specificity is the direct repeat sequence, which is to be appropriately linked to such guides. In particular, this implies that the direct repeat sequences are designed dependent on the origin of the enzyme. Structural data available for validated dead guide sequences may be used for designing CRISPR-Cas specific equivalents. Structural similarity between, e.g., the orthologous nuclease domains of two or more CRISPR-Cas proteins may be used to transfer design equivalent dead guides. Thus, the dead guide herein may be appropriately modified in length and sequence to reflect such CRISPR-Cas specific equivalents, allowing for formation of the CRISPR-Cas complex and successful binding to the target RNA, while at the same time, not allowing for successful nuclease activity.

Dead guides allow one to use gRNA or crRNA as a means for gene targeting, without the consequence of nuclease activity, while at the same time providing directed means for activation or repression. Guide RNA or crRNA comprising a dead guide may be modified to further include elements in a manner which allow for activation or repression of gene activity, in particular protein adaptors (e.g. aptamers) as described herein elsewhere allowing for functional placement of gene effectors (e.g. activators or repressors of gene activity). One example is the incorporation of aptamers, as explained herein and in the state of the art. By engineering the gRNA or crRNA comprising a dead guide to incorporate protein-interacting aptamers (Konermann et al., “Genome-scale transcription activation by an engineered CRISPR-Cas9 complex,” doi:10.1038/nature14136, incorporated herein by reference), one may assemble multiple distinct effector domains. Such may be modeled after natural processes.

### Determination of PAM

Determination of PAM can be performed as follows. This experiment closely parallels similar work in E. coli for the heterologous expression of StCas9 (Sapranauskas, R. et al. Nucleic Acids Res 39, 9275-9282 (2011)). Applicants introduce a plasmid containing both a PAM and a resistance gene into the heterologous E. coli, and then plate on the corresponding antibiotic. If there is DNA cleavage of the plasmid, Applicants observed no viable colonies.

In further detail, the assay is as follows for a DNA target. Two E. coli strains are used in this assay. One carries a plasmid that encodes the endogenous effector protein locus from the bacterial strain. The other strain carries an empty plasmid (e.g. pACYC184, control strain). All possible 7 or 8 bp PAM sequences are presented on an antibiotic resistance plasmid (pUC19 with ampicillin resistance gene). The PAM is located next to the sequence of proto-spacer 1 (the DNA target to the first spacer in the endogenous effector protein locus). Two PAM libraries were cloned. One has a 8 random bp 5′ of the proto-spacer (e.g. total of 65536 different PAM sequences=complexity). The other library has 7 random bp 3′ of the proto-spacer (e.g. total complexity is 16384 different PAMs). Both libraries were cloned to have in average 500 plasmids per possible PAM. Test strain and control strain were transformed with 5′PAM and 3′PAM library in separate transformations and transformed cells were plated separately on ampicillin plates. Recognition and subsequent cutting/interference with the plasmid renders a cell vulnerable to ampicillin and prevents growth. Approximately 12 h after transformation, all colonies formed by the test and control strains where harvested and plasmid DNA was isolated. Plasmid DNA was used as template for PCR amplification and subsequent deep sequencing. Representation of all PAMs in the untransformed libraries showed the expected representation of PAMs in transformed cells. Representation of all PAMs found in control strains showed the actual representation. Representation of all PAMs in test strain showed which PAMs are not recognized by the enzyme and comparison to the control strain allows extracting the sequence of the depleted PAM.

### Base Editing

The present disclosure also provides for a base editing system. In general, such a system may comprise a deaminase (e.g., an adenosine deaminase or cytidine deaminase) fused with a Cas protein described herein. The Cas protein may be a dead Cas protein or a Cas nickase protein. In certain examples, the system comprises a mutated form of an adenosine deaminase fused with a dead CRISPR-Cas or CRISPR-Cas nickase. The mutated form of the adenosine deaminase may have both adenosine deaminase and cytidine deaminase activities.

In some examples, the present disclosure provides an engineered, non-naturally occurring composition comprising: the nuclei acid-guided nuclease that is catalytically inactive, a nucleotide deaminase associated with or otherwise capable of forming a complex with the Cas protein, and a single guide molecule capable of forming a complex with the Cas protein and directing site-specific binding at a target sequence. A base-editing system may comprise a deaminase (e.g., an adenosine deaminase or cytidine deaminase) fused with a nucleic acid-guided nuclease or a variant thereof. In some cases, the target polynucleotide is edited at one or more bases to introduce a G→A or C→T mutation.

In one aspect, the present disclosure provides an engineered adenosine deaminase. The engineered adenosine deaminase may comprise one or more mutations herein. In some embodiments, the engineered adenosine deaminase has cytidine deaminase activity. In certain examples, the engineered adenosine deaminase has both cytidine deaminase activity and adenosine deaminase. In some cases, the modifications by base editors herein may be used for targeting post-translational signaling or catalysis. In some embodiments, compositions herein comprise nucleotide sequence comprising encoding sequences for one or more components of a base editing system. A base-editing system may comprise a deaminase (e.g., an adenosine deaminase or cytidine deaminase) fused with a Cas protein or a variant thereof.

In some cases, the adenosine deaminase is double-stranded RNA-specific adenosine deaminase (ADAR). Examples of ADARs include those described Yiannis A Savva et al., The ADAR protein family, Genome Biol. 2012; 13(12): 252, which is incorporated by reference in its entirety. In some examples, the ADAR may be hADAR1. In certain examples, the ADAR may be hADAR2. The sequence of hADAR2 may be that described under Accession No. AF525422.1.

In some cases, the deaminase may be a deaminase domain, e.g., a deaminase domain of ADAR (“ADAR-D”). In one example, the deaminase may be the deaminase domain of hADAR2 (“hADAR2-D), e.g., as described in Phelps K J et al., Recognition of duplex RNA by the deaminase domain of the RNA editing enzyme ADAR2. Nucleic Acids Res. 2015 January; 43(2):1123-32, which is incorporated by reference herein in its entirety. In a particular example, the hADAR2-D has a sequence comprising amino acid 299-701 of hADAR2-D, e.g., amino acid 299-701 of the sequence under Accession No. AF525422.1.

In certain examples, the system comprises a mutated form of an adenosine deaminase fused with a dead CRISPR-Cas or CRISPR-Cas nickase. The mutated form of the adenosine deaminase may have both adenosine deaminase and cytidine deaminase activities. In some embodiments, the adenosine deaminase may comprise one or more of the mutations: E488Q based on amino acid sequence positions of hADAR2-D, and mutations in a homologous ADAR protein corresponding to the above. In some embodiments, the adenosine deaminase may comprise one or more of the mutations: E488Q, V351G, based on amino acid sequence positions of hADAR2-D, and mutations in a homologous ADAR protein corresponding to the above. In some embodiments, the adenosine deaminase may comprise one or more of the mutations: E488Q, V351G, S486A, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, S582T, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, S582T, V440I based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, S582T, V440I, S495N based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, S582T, V440I, S495N, K418E based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, S582T, V440I, S495N, K418E, S661T based on amino acid sequence positions of hADAR2-D, and mutations in a homologous ADAR protein corresponding to the above. In some examples, provided herein includes a mutated adenosine deaminase e.g., an adenosine deaminase comprising one or more mutations of E488Q, V351G, S486A, T375S, S370C, P462A, N597I, L332I, I398V, K350I, M383L, D619G, S582T, V440I, S495N, K418E, S661T, fused with a dead CRISPR-Cas protein or CRISPR-Cas nickase. In some examples, provided herein includes a mutated adenosine deaminase e.g., an adenosine deaminase comprising E488Q, V351G, S486A, T375S, S370C, P462A, N597I, L332I, I398V, K350I, M383L, D619G, S582T, V440I, S495N, K418E, and S661T, fused with a dead CRISPR-Cas protein or a CRISPR-Cas nickase. In some examples, provided herein includes a mutated adenosine deaminase e.g., an adenosine deaminase comprising E488Q, V351G, S486A, T375S, S370C, P462A, N597I, L332I, I398V, K350I, M383L, D619G, S582T, V440I, S495N, K418E, S661T, and S375N fused with a dead CRISPR-Cas protein or a CRISPR-Cas nickase.

In some embodiments, the adenosine deaminase may be a tRNA-specific adenosine deaminase or a variant thereof. In some embodiments, the adenosine deaminase may comprise one or more of the mutations: W23L, W23R, R26G, H36L, N37S, P48S, P48T, P48A, I49V, R51L, N72D, L84F, S97C, A106V, D108N, H123Y, G125A, A142N, S 146C, D147Y, R152H, R152P, E155V, I156F, K157N, K161T, based on amino acid sequence positions of E. coli TadA, and mutations in a homologous deaminase protein corresponding to the above. In some embodiments, the adenosine deaminase may comprise one or more of the mutations: D108N based on amino acid sequence positions of E. coli TadA, and mutations in a homologous deaminase protein corresponding to the above. In some embodiments, the adenosine deaminase may comprise one or more of the mutations: A106V, D108N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, based on amino acid sequence positions of E. coli TadA, and mutations in a homologous deaminase protein corresponding to the above. In some embodiments, the adenosine deaminase may comprise one or more of the mutations: A106V, D108N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, A142N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, A142N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, W23R, P48A, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, W23R, P48A, A142N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, W23R, P48A, R152P, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, W23R, P48A, R152P, A142N, based on amino acid sequence positions of E. coli TadA, and mutations in a homologous deaminase protein corresponding to the above.

In some examples, the base editing systems may comprise an intein-mediated trans-splicing system that enables in vivo delivery of a base editor, e.g., a split-intein cytidine base editors (CBE) or adenine base editor (ABE) engineered to trans-splice. Examples of the such base editing systems include those described in Colin K. W. Lim et al., Treatment of a Mouse Model of ALS by In Vivo Base Editing, Mol Ther. 2020 Jan. 14. pii: S1525-0016(20)30011-3. doi: 10.1016/j.ymthe.2020.01.005; and Jonathan M. Levy et al., Cytosine and adenine base editing of the brain, liver, retina, heart and skeletal muscle of mice via adeno-associated viruses, Nature Biomedical Engineering volume 4, pages 97-110(2020), which are incorporated by reference herein in their entireties.

Examples of base editing systems include those described in WO2019071048 (e.g. paragraphs [0933]-0938]), WO2019084063 (e.g., paragraphs [0173]-[0186], [0323]-[0475], [0893]-[1094]), WO2019126716 (e.g., paragraphs [0290]-[0425], [1077]-[1084]), WO2019126709 (e.g., paragraphs [0294]-[0453]), WO2019126762 (e.g., paragraphs [0309]-[0438]), WO2019126774 (e.g., paragraphs [0511]-[0670]), Cox DBT, et al., RNA editing with CRISPR-Cas13, Science. 2017 Nov. 24; 358(6366):1019-1027; Abudayyeh 00, et al., A cytosine deaminase for programmable single-base RNA editing, Science 26 Jul. 2019: Vol. 365, Issue 6451, pp. 382-386; Gaudelli N M et al., Programmable base editing of A•T to G•C in genomic DNA without DNA cleavage, Nature volume 551, pages 464-471 (23 Nov. 2017); Komor A C, et al., Programmable editing of a target base in genomic DNA without double-stranded DNA cleavage. Nature. 2016 May 19; 533(7603):420-4; Jordan L. Doman et al., Evaluation and minimization of Cas9-independent off-target DNA editing by cytosine base editors, Nat Biotechnol (2020). doi.org/10.1038/s41587-020-0414-6; and Richter M F et al., Phage-assisted evolution of an adenine base editor with improved Cas domain compatibility and activity, Nat Biotechnol (2020). doi.org/10.1038/s41587-020-0453-z, which are incorporated by reference herein in their entireties.

### Regulation of Post-Translational Modification of Gene Products

In some cases, base editing may be used for regulating post-translational modification of a gene products. In some cases, an amino acid residue that is a post-translational modification site may be mutated by base editing to an amino residue that cannot be modified. Examples of such post-translational modifications include disulfide bond formation, glycosylation, lipidation, acetylation, phosphorylation, methylation, ubiquitination, sumoylation, or any combinations thereof.

In some embodiments, the base editors herein may regulate Stat3/IRF-5 pathway, e.g., for reduction of inflammation. For example, phosphorylation on Tyr705 of Stat3, Thr10, Ser158, Ser309, Ser317, Ser451, and/or Ser462 of IRF-5 may be involved with interleukin signaling. Base editors herein may be used to mutate one or more of these procreation sites for regulating immunity, autoimmunity, and/or inflammation.

In some embodiments, the base editors herein may regulate insulin receptor substrate (IRS) pathway. For example, phosphorylation on Ser265, Ser302, Ser325, Ser336, Ser358, Ser407, and/or Ser408 may be involved in regulating (e.g., inhibit) ISR pathway. Alternatively, or additionally, Serine 307 in mouse (or Serine 312 in human) may be mutated so the phosphorylation may be regulated. For example, Serine 307 phosphorylation may lead to degradation of IRS-1 and reduce MAPK signaling. Serine 307 phosphorylation may be induced under insulin insensitivity conditions, such as insulin overstimulation and/or TNFα treatment. In some examples, S307F mutation may be generated for stabilizing the interaction between IRS-1 and other components in the pathway. Base editors herein may be used to mutate one or more of these procreation sites for regulating IRS pathway.

### Regulation of Stability of Gene Products

In some embodiments, base editing may be used for regulating the stability of gene products. For example, one or more amino acid residues that regulate protein degradation rates may be mutated by the base editors herein. In some cases, such amino acid residues may be in a degron. A degron may refer to a portion of a protein involved in regulating the degradation rate of the protein. Degrons may include short amino acid sequences, structural motifs, and exposed amino acids (e.g., lysine or arginine). Some protein may comprise multiple degrons. The degrons be ubiquitin-dependent (e.g., regulating protein degradation based on ubiquitination of the protein) or ubiquitin-independent.

In some cases, the based editing may be used to mutate one or more amino acid residues in a signal peptide for protein degradation. In some examples, the signal peptide may be a PEST sequence, which is a peptide sequence that is rich in proline (P), glutamic acid (E), serine (S), and threonine (T). For example, the stability of NANOG, which comprises a PEST sequence, may be increased, e.g., to promote embryonic stem cell pluripotency.

In some examples, the base editors may be used for mutating SMN2 (e.g., to generate S270A mutilation) to increase stability of the SMN2 protein, which is involved in spinal muscular atrophy. Other mutations in SMN2 that may be generated by based editors include those described in Cho S. et al., Genes Dev. 2010 Mar. 1; 24(5): 438-442. In certain examples, the base editors may be used for generating mutations on IκBα, as described in Fortmann K T et al., J Mol Biol. 2015 Aug. 28; 427(17): 2748-2756. Target sites in degrons may be identified by computational tools, e.g., the online tools provided on slim.ucd.ie/apc/index.php. Other targets include Cdc25A phosphatase.

Examples of Genes that can be Targeted by Base Editors

In some examples, the base editors may be used for modifying PCSK9. The base editors may introduce stop codons and/or disease-associated mutations that reduce PCSK9 activity. The base editing may introduce one or more of the following mutations in PCSK9: R46L, R46A, A53V, A53A, E57K, Y142X, L253F, R237W, H391N, N425S, A443T, I474V, I474A, Q554E, Q619P, E670G, E670A, C679X, H417Q, R469W, E482G, F515L, and/or H553R.

In some examples, the base editors may be used for modifying ApoE. The base editors may target ApoE in synthetic model and/or patient-derived neurons (e.g., those derived from iPSC). The targeting may be tested by sequencing.

In some examples, the base editors may be used for modifying Stat1/3. The base editor may target Y705 and/or S727 for reducing Stat1/3 activation. The base editing may be tested by luciferase-based promoter. Targeting Stat1/3 by base editing may block monocyte to macrophage differentiation, and inflammation in response to ox-LDL stimulation of macrophages.

In some examples, the base editors may be used for modifying TFEB (transcription factor for EB). The base editor may target one or more amino acid residues that regulate translocation of the TFEB. In some cases, the base editor may target one or more amino acid residues that regulate autophagy.

In some examples, the base editors may be used for modifying ornithine carbamoyl transferase (OTC). Such modification may be used for correct ornithine carbamoyl transferase deficiency. For example, base editing may correct Leu45Pro mutation by converting nucleotide 134C to U.

In some examples, the base editors may be used for modifying Lipin1. The base editor may target one or more serine's that can be phosphorylated by mTOR. Base editing of Lipin1 may regulate lipid accumulation. The base editors may target Lipin1 in 3T3L1 preadipocyte model. Effects of the base editing may be tested by measuring reduction of lipid accumulation (e.g., via oil red).

### Prime Editing

In another aspect, the present disclosure provides compositions and systems for prime editing. In some embodiments, the Cas protein herein may be used for prime editing. In some cases, the Cas protein may be a nickase, e.g., a DNA nickase. The Cas may be a Cas9. The Cas9 may be a dCas9-t. In some cases, the Cas protein has one or more mutations. In some examples, the Cas protein may be a Cas9 from or derived from Streptococcus pyogenes and comprises the H840A mutation. In some examples, the Cas9 is from or derived from Streptococcus pyogenes and comprises the D10A mutations. In some examples, the Cas9 has mutation(s) corresponding to D10A or H840A.

The Cas protein may be associated with a reverse transcriptase. The reverse transcriptase may be fused to the C-terminus of a Cas9 protein. Alternatively, or additionally, the reverse transcriptase may be fused to the N-terminus of a Cas9 protein. The fusion may be via a linker and/or an adaptor protein.

A reverse transcriptase domain may be a reverse transcriptase or a fragment thereof. A wide variety of reverse transcriptases (RT) may be used in alternative embodiments of the present disclosure, including prokaryotic and eukaryotic RT, provided that the RT functions within the host to generate a donor polynucleotide sequence from the RNA template. If desired, the nucleotide sequence of a native RT may be modified, for example using known codon optimization techniques, so that expression within the desired host is optimized. A reverse transcriptase (RT) is an enzyme used to generate complementary DNA (cDNA) from an RNA template, a process termed reverse transcription. Reverse transcriptases are used by retroviruses to replicate their genomes, by retrotransposon mobile genetic elements to proliferate within the host genome, by eukaryotic cells to extend the telomeres at the ends of their linear chromosomes, and by some non-retroviruses such as the hepatitis B virus, a member of the Hepadnaviridae, which are dsDNA-RT viruses. Retroviral RT has three sequential biochemical activities: RNA-dependent DNA polymerase activity, ribonuclease H, and DNA-dependent DNA polymerase activity. Collectively, these activities enable the enzyme to convert single-stranded RNA into double-stranded cDNA. In certain embodiments, the RT domain of a reverse transcriptase is used in the present disclosure. The domain may include only the RNA-dependent DNA polymerase activity. In some examples, the RT domain is non-mutagenic, i.e., dose not cause mutation in the donor polynucleotide (e.g., during the reverse transcriptase process). In some cases, In some examples, the RT domain may be non-retron RT, e.g., a viral RT or a human endogenous RTs. In some examples, the RT domain may be retron RT or DGRs RT. In some example, the RT may be less mutagenic than a counterpart wildtype RT. In some embodiments, the RT herein is not mutagenic.

In some examples, the reverse transcriptase may be an M-MLV reverse transcriptase or variant thereof. The M-MLV reverse transcriptase variant may comprise one or more mutations. For the examples, the M-MLV reverse transcriptase may comprise D200N, L603W, and T330P. In another example, the M-MLV reverse transcriptase may comprise D200N, L603W, T330P, T306K, and W313F. In a particular example, the fusion of Cas9 and reverse transcriptase is Cas9 (H840A) fused with M-MLV reverse transcriptase (D200N+L603W+T330P+T306K+W313F).

In some embodiments, the Cas protein herein may target DNA using a guide RNA containing a binding sequence that hybridizes to the target sequence on the DNA. The guide RNA may further comprise an editing sequence that contains new genetic information that replaces target DNA nucleotides. The small sizes of the Cas proteins herein may allow easier packaging and delivery of the prime editing system, e.g., with a viral vector, e.g., AAV or lentiviral vector.

A single-strand break (a nick) may be generated on the target DNA by the Cas9 protein at the target site to expose a 3′-hydroxyl group, thus priming the reverse transcription of an edit-encoding extension on the guide directly into the target site. These steps may result in a branched intermediate with two redundant single-stranded DNA flaps: a 5′ flap that contains the unedited DNA sequence, and a 3′ flap that contains the edited sequence copied from the guide RNA. The 5′ flaps may be removed by a structure-specific endonuclease, e.g., FEN122, which excises 5′ flaps generated during lagging-strand DNA synthesis and long-patch base excision repair. The non-edited DNA strand may be nicked to induce bias DNA repair to preferentially replace the non-edited strand. Examples of prime editing systems and methods include those described in Anzalone A V et al., Search-and-replace genome editing without double-strand breaks or donor DNA, Nature. 2019 Oct. 21. doi: 10.1038/s41586-019-1711-4, which is incorporated by reference herein in its entirety.

The Cas proteins may be used to prime-edit a single nucleotide on a target DNA. Alternatively or additionally, the Cas9 proteins may be used to prime-edit at least 2, at least 3, at least 4, at least 5, at least 6, at least 7, at least 8, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 40, at least 50, at least 60, at least 70, at least 80, at least 90, at least 100, at least 200, at least 300, at least 400, at least 500, at least 600, at least 700, at least 800, at least 900, or at least 10000 nucleotides on a target DNA.

In certain embodiments, the reverse transcriptase is Human immunodeficiency virus (HIV) RT, Avian myoblastosis virus (AMV) RT, Moloney murine leukemia virus (M-MLV) RT a group II intron RT, a group II intron-like RT, or a chimeric RT. In certain embodiments, the RT comprises modified forms of these RTs, such as, engineered variants of Avian myoblastosis virus (AMV) RT, Moloney murine leukemia virus (M-MLV) RT, or Human immunodeficiency virus (HIV) RT (see, e.g., Anzalone, et al., Search-and-replace genome editing without double-strand breaks or donor DNA, Nature. 2019 December; 576(7785): 149-157).

In some examples, the compositions and systems may comprise the Cas protein herein; a reverse transcriptase (RT) polypeptide connected to or otherwise capable of forming a complex with the Cas protein; and a guide molecule capable of forming a CRISPR-Cas complex with the Cas protein and comprising: a guide sequence capable of directing site-specific binding of the CRISPR-Cas complex to a target sequence of a target polynucleotide; a 3′ binding site region capable of binding to a cleaved upstream strand of the target polynucleotide; and a RT template sequence encoding an extended sequence, wherein the extended sequence comprises a variant region and a 3′ homologous sequence capable of hybridization to the downstream cleaved strand of the target polynucleotide.

In some examples, the compositions and systems the Cas protein herein; a reverse transcriptase (RT) polypeptide connected to or otherwise capable of forming a complex with the Cas protein; a first guide molecule capable of forming a first CRISPR-Cas complex with the Cas protein and comprising: a guide sequence capable of directing site-specific binding of the first CRISPR-Cas complex to a first target sequence of a target polynucleotide; a first binding site region capable of binding to a cleaved or nicked strand of the target polynucleotide; and a RT template sequence encoding a first extended sequence; a second guide molecule capable of forming a second CRISPR-Cas complex with the Cas protein and comprising: a guide sequence capable of directing site specific binding of the second CRISPR-Cas complex to a second target sequence of the target polynucleotide; a second binding site region capable of binding to a cleaved or nicked strand of the target polynucleotide; and a RT template sequence encoding a second extended sequence.

In some cases, the compositions and systems may further comprise: a donor template; a third guide sequence capable of forming a CRISPR-Cas complex with the Cas protein and comprising: a guide sequence capable of directing site-specific binding to a target sequence on the donor template; a third binding region capable of binding to a cleaved or nicked strand of the donor template; and a RT template encoding a third extended region complementary to the first extended region generated on the target polynucleotide: and a fourth guide sequence capable of forming a CRISPR-Cas complex with the Cas protein and comprising: a guide sequence capable of directing site-specific binding to a second target sequence on the donor template; a fourth binding region capable of binding to a cleaved or nicked strand of the donor template; and a RT template encoding a fourth extended region complementary to the second extended region generated on the target polynucleotide.

In some cases, the compositions and systems may further comprise a site-specific recombinase, and wherein the first and second extended regions are complementary to each other and introduce a serine integrase recombination site; and a donor molecule comprising a donor sequence for insertion into the target polypeptide and the complementary recombination site to the serine integrase recombination site.

In some examples, the compositions and systems may further comprise a recombinase. The recombinase is connected to or otherwise capable of forming a complex with the Cas protein. In certain embodiments, the complex is capable of inserting a recombination site in the DNA loci of interest by extension of RT templates that encode for the recombination site on the 3′ extension of the guide sequences by the reverse transcriptase. In certain embodiments, a donor template comprising a compatible recombination site is provided that can recombine unidirectionally with the inserted recombination site when a recombinase specific for the recombination site is also provided. In certain embodiments, the donor template is a plasmid comprising the complementary recombination site and any sequence for insertion at the DNA loci of interest. In certain embodiments, the recombinase is connected to or capable of forming a complex with the CRISPR enzyme, such that all of the enzymatic proteins are brought into contact at the loci of interest. In certain embodiments, the recombinase is codon optimized for eukaryotic cells (described further herein). In certain embodiments, the recombinase includes a NLS (described further herein). In certain embodiments, the recombinase is provided as a separate protein. The separate recombinase may form a dimer and bind to the donor template recombination site. The recombinase may be targeted to the loci of interest as a result of the insertion of the compatible recombination site that is also recognized by the recombinase. Thus, the recombinase may recognize the recombination site inserted at the DNA loci of interest and the recombination site on the donor and be targeted to the DNA loci of interest without any additional modifications to the recombinase.

In certain embodiments, a second CRISPR complex connected to a recombinase is targeted to the DNA loci of interest. In certain embodiments, the second CRISPR complex comprises a dead Cas protein (dCas, described further herein), such that the recombinase is targeted to the DNA loci of interest, but the target sequence is not further cleaved. In certain embodiments, the dCas targets a sequence generated only after the insertion of the recombination site. In certain embodiments, the recombinase recognizes and binds to the donor template recombination site and the inserted recombination site. In certain embodiments, the recombinase forms a dimer with a recombinase provided as a separate protein.

As used herein, the term “Recombinase” refers to an enzyme that catalyzes recombination between two or more recombination sites (e.g., an acceptor and donor site). Recombinases useful in the present invention catalyze recombination at specific recombination sites which are specific polynucleotide sequences that are recognized by a particular recombinase. “Uni-directional recombinases” or “integrases” refer to recombinase enzymes whose recognition sites are destroyed after the recombination has taken place. The term “integrase” refers to a type of recombinase. In other words, the sequence recognized by the recombinase is changed into one that is not recognized by the recombinase upon recombination. As a result, once a sequence is subjected to recombination by the uni-directional recombinase, the continued presence of the recombinase cannot reverse the previous recombination event.

“Recombination sites” are specific polynucleotide sequences that are recognized by the recombinase enzymes described herein. Typically, two different sites are involved (in regards to recombination termed “complementary sites”), one present in the target nucleic acid (e.g., a chromosome or episome of a eukaryote) and another on the nucleic acid that is to be integrated at the target recombination site. The terms “attB” and “attP,” which refer to attachment (or recombination) sites originally from a bacterial target (attachment site of bacteria) and a phage donor (attachment site of phage), respectively, are used herein although recombination sites for particular enzymes may have different names. The two attachment sites can share as little sequence identity as a few base pairs. The recombination sites typically include left and right arms separated by a core or spacer region. Thus, an attB recombination site consists of BOB′, where B and B′ are the left and right arms, respectively, and O is the core region. Similarly, attP is POP′, where P and P′ are the arms and O is again the core region. Upon recombination between the attB and attP sites, and concomitant integration of a nucleic acid at the target, the recombination sites that flank the integrated DNA are referred to as “attL” and “aatR.” The attL and attR sites, using the terminology above, thus consist of BOP′ and POB′, respectively. In some representations herein, the “O” is omitted and attB and attP, for example, are designated as BB′ and PP′, respectively.

### Retrotransposons

The systems and compositions herein may comprise a nucleic acid-guided nuclease, one or more guide molecules, and one or more components of a retrotransposon, e.g., a non-LTR retrotransposon. The one or more components of a retrotransposon include a retrotransposon protein and retrotransposon RNA. The systems and compositions may be used to insert a donor polynucleotide to a target polynucleotide. The systems and compositions may further comprise a donor polynucleotide.

In some examples, the present disclosure provides an engineered, non-naturally occurring composition comprising: a nucleic acid-guided nuclease, a non-LTR retrotransposon protein associated with or otherwise capable of forming a complex with the nucleic acid-guided nuclease; a single guide molecule capable of forming a complex with the nucleic acid-guided nuclease and directing site-specific binding to a target sequence of a target polynucleotide. The composition may further comprise a donor construct comprising a donor polynucleotide for insertion to the target polynucleotide and located between two binding elements capable of forming a complex with the non-LTR retrotransposon protein. In some cases, the nucleic acid-guided nuclease is engineered to have nickase activity.

In some examples, the nucleic acid-guided nuclease is fused to the N-terminus of the non-LTR retrotransposon protein. In some examples, the nucleic acid-guided nuclease is fused to the C-terminus of the non-LTR retrotransposon protein.

The guides may direct the fusion protein to a target sequence 5′ of the targeted insertion site, and wherein the nucleic acid-guided nuclease generates a double-strand break at the targeted insertion site. The guides may direct the fusion protein to a target sequence 3′ of the targeted insertion site, and wherein the nucleic acid-guided nuclease generates a double-strand break at the targeted insertion site.

The donor polynucleotide may further comprise a polymerase processing element to facilitate 3′ end processing of the donor polynucleotide sequence. The polymerase may be a DNA polymerase, e.g., DNA polymerase I. In some examples, the polymerase may be an RNA polymerase.

In some examples, the donor polynucleotide further comprises a homology region to the target sequence on the 5′ end of the donor construct, the 3′ end of the donor construct, or both. In some examples, the homology region is from 1 to 50, from 5 to 30, from 8 to 25, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 base pairs in length.

Native or wild-type non-LTR retrotransposons encode the protein machinery necessary for their self-mobilization. The non-LTR retrotransposon element comprises a DNA element integrated into a host genome. This DNA element may encode one or two open reading frames (ORFs). For example, the R2 element of Bombyx mori encodes a single ORF containing reverse transcriptase (RT) activity and a restriction enzyme-like (REL) domain. L1 elements encode two ORFs, ORF1 and ORF2. ORF1 contains a leucine zipper domain involved in protein-protein interactions and a C-terminal nucleic acid binding domain. ORF2 has a N-terminal apurinic/apyrimidinic endonuclease (APE), a central RT domain, and a C-terminal cysteine histidine rich domain. An example replicative cycle of a non-LTR retrotransposon may comprise transcription of the full-length retrotransposon element to generate an mRNA active element (retrotransposon RNA). The active element mRNA is translated to generate the encoded retrotransposon proteins or polypeptides. A ribonucleoprotein complex comprising the active element and retrotransposon protein or polypeptide is formed and this RNP facilitates integration of the active element into the genome. The RNA-transposase complex nicks the genome. The 3′ end of the nicked DNA serves as a primer to allow the reverse transcription of the transposon RNA into cDNA. Fourth, the transposase proteins integrate the cDNA into the genome.

Elements of these systems may be engineered to work within the context of the invention. For example a non-LTR retrotransposon polypeptide may be fused to a site-specific nuclease. The binding elements that allow a non-LTR retrotransposon polypeptide to bind to the native retrotransposon DNA element, may be engineered into a donor construct to facilitate entry of a donor polynucleotide sequence into a target polypeptide.

In the present disclosure the protein component of the non-LTR retrotransposon may be connected to or otherwise engineered to form a complex with a site-specific nuclease. The retrotransposon RNA may be engineered to encode a donor polynucleotide sequence. Thus, in certain example embodiments, the nucleic acid-guided nuclease, via formation of a nucleic acid-guided nuclease complex with a guide sequence, directs the retrotransposon complex (e.g. the retrotransposon polypeptide(s) and retrotransposon RNA to a target sequence in a target polynucleotide, where the retrotransposon RNP complex facilitates integration of the donor polynucleotide sequence into the target polynucleotide. Accordingly, the one or more non-LTR retrotransposon components may comprise retrotransposon polypeptides, or function domains thereof, that facilitate binding of the retrotransposon RNA, reverse transcription of the retrotransposon RNA into cDNA, and/or integration of the donor polynucleotide into the target polynucleotide, as well as retrotransposon RNA elements modified to encode the donor polynucleotide sequence.

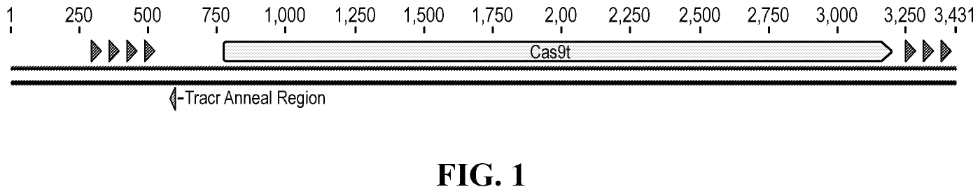

Examples non-LTR retrotransposons include CRE, R2, R4, L1, RTE, Tad, R1, LOA, I, Jockey, CR1 (see FIG. 1). In one example, the non-LTR retrotransposon is R2. In another example, the non-LTR retrotransposon is L1. Examples of non-LTR retrotransposons may include those described in Christensen S M et al., RNA from the 5′ end of the R2 retrotransposon controls R2 protein binding to and cleavage of its DNA target site, Proc Natl Acad Sci USA. 2006 Nov. 21; 103(47):17602-7; Eickbush T H et al, Integration, Regulation, and Long-Term Stability of R2 Retrotransposons, Microbiol Spectr. 2015 April; 3(2):MDNA3-0011-2014. doi: 10.1128/microbiolspec.MDNA3-0011-2014; Han J S, Non-long terminal repeat (non-LTR) retrotransposons: mechanisms, recent developments, and unanswered questions, Mob DNA. 2010 May 12; 1(1):15. doi: 10.1186/1759-8753-1-15; Malik H S et al., The age and evolution of non-LTR retrotransposable elements, Mol Biol Evol. 1999 June; 16(6):793-805, which are incorporated by reference herein in their entireties.

Examples of the non-LTR retrotransposon polypeptides also include R2 from Clonorchis sinensis, or Zonotrichia albicollis.

A non-LTR retrotransposon may comprise multiple retrotransposon polypeptides or polynucleotides encoding same. In some embodiments, the retrotransposon polypeptides may form a complex. For example, a non-LTR retrotransposon is a dimer, e.g., comprising two retrotransposon polypeptides forming a dimer. The dimer subunits may be connected or form a tandem fusion. A nucleic acid-guided nuclease may be associate with (e.g., connected to) one or more subunits of such complex. In some examples, the non-LTR retrotransposon is a dimer of two retrotransposon polypeptides; one of the retrotransposon polypeptides comprises nuclease or nickase activity and is connected with a nucleic acid-guided nuclease.

The retrotransposon polypeptides may comprise one or more modifications to, for example, enhance specificity or efficiency of donor polynucleotide recognition, target-primed template recognition (TPTR). The retrotransposon polypeptides may also comprise one or more truncations or excisions to remove domains or regions of wild-type protein to arrive at a minimal polypeptide that retain donor polynucleotide recognition and TPTR. In some example embodiments, the native endonuclease activity may be mutated to eliminate endonuclease activity.

In certain example embodiments, the modifications or truncations of the non-LTR retrotransposon peptide may be in a zinc finger region, a Myb region, a basic region, a reverse transcriptase domain, a cysteine-histidine rich motif, or an endonuclease domain.

A non-LTR retrotransposon may comprise polynucleotide encoding one or more retrotransposon RNA molecules. The polynucleotide may comprise one or more regulatory elements. The regulatory elements may be promoters. The regulatory elements and promoters on the polynucleotides include those described throughout this application. For example, the polynucleotide may comprise a pol2 promoter, a pol3 promoter, or a T7 promoter.

In some cases, the polynucleotide encodes a retrotransposon RNA with at least a portion of its sequence complementary to a target sequence. For example, the 3′ end of the retrotransposon RNA may be complementary to a target sequence. The RNA may be complementary to a portion of a nicked target sequence. In some embodiments, a retrotransposon RNA may comprise one or more donor polynucleotides. In certain cases, a retrotransposon RNA may encode one or more donor polynucleotides.

A retrotransposon RNA may be capable of binding to a retrotransposon polypeptide. Such retrotransposon RNA may comprise one or more elements for binding to the retrotransposon polypeptide. Examples of binding elements include hairpin structures, pseudoknots (e.g., a nucleic acid secondary structure containing at least two stem-loop structures in which half of one stem is intercalated between the two halves of another stem), stem loops, and bulges (e.g., unpaired stretches of nucleotides located within one strand of a nucleic acid duplex). In certain examples, the retrotransposon RNA comprises one or more hairpin structures. In some examples, the retrotransposon RNA comprises one or more pseudoknots. In certain examples, a retrotransposon RNA comprises a sequence encoding a donor polynucleotide and one or more binding elements for forming a complex with the retrotransposon polypeptide. The binding elements may be located on the 5′ end or the 3′ end.

In some embodiments, a retrotransposon RNA comprises a region capable of hybridizing with an overhang of a target polynucleotide at the target site. The overhang may be a stretch of single-stranded DNA. The overhang may function as a primer for reverse transcription of at least a portion of the retrotransposon RNA to a cDNA. In some cases, a region of the cDNA may be capable of hybridizing a second overhang of the target polynucleotide. The second overhang may function as a primer for the synthesis of a second strand to generate a double-stranded cDNA. The cDNA may comprise a donor polynucleotide sequence. The two overhangs may be from different strands of the target polynucleotide.

### Guided Excision-Transposition Systems

Embodiments disclosed herein also provide an engineered or non-natural guided excision-transposition system. The engineered or non-natural guided excision-transposition system may comprise one or more components of a CRISPR-Cas system herein (e.g., one or more Cas9-t and one or more guide molecules) and one or more components of a Class II transposon. The components of the CRISPR-Cas system can direct the Class II transposon component(s) to retrotransposon to a target nucleic acid sequence and guide its transposition into a recipient polynucleotide.

For example, the engineered or non-natural guided excision-transposition systems that can include (a) a first Cas protein; (b) a first Class II transposon polypeptide coupled to or otherwise capable of complexing with the first Cas protein; (c) a first guide molecule capable of forming a CRISPR-Cas complex with the first Cas protein and directing site-specific binding to a first target sequence of a first target polynucleotide; (d) a second Cas protein; (e) a second Class II transposon polypeptide coupled to or otherwise capable of complexing with the second Cas protein; (f) a second guide molecule capable of forming a CRISPR-Cas complex with the first Cas protein and directing site-specific binding to a second target sequence of the first target polynucleotide; and (g) a Class II transposon polynucleotide comprising the first target polynucleotide and is capable of forming a complex with the first and second Cas protein, the first and second guide molecules, and the first and second Class II transposon polypeptides.

In some embodiments, the engineered or non-natural guided excision-transposition system can include (h) a third guide molecule capable of complexing with the first Cas protein and directing site-specific binding to a first target sequence of a second target polynucleotide, wherein the third guide molecule is optionally coupled to the first Cas protein; (i) optionally, a first guide molecule polynucleotide that encodes the third guide molecule; (j) a fourth guide molecule capable of complexing with the second Cas protein and directing site-specific binding to a second target sequence of the second target polynucleotide, wherein the fourth guide molecule is optionally coupled to the second Cas protein; and (k) optionally, a second guide molecule polynucleotide that encodes the fourth guide molecule.

In some embodiments, the first and the second Class II transposon polypeptides are capable of excising the first target polynucleotide from the Class II transposon polynucleotide. In some embodiments, the first and the second Class II transposon polypeptides are capable of transposing the first target polynucleotide in the second target polynucleotide. In some embodiments, the first target polynucleotide does not include one or more Class II transposon long terminal repeats.

The engineered or non-natural guided excision-transposition systems described herein can be based on a Class II transposon or Class II transposon system. The engineered or non-natural guided excision-transposition system may include a first target polynucleotide, also referred to as a donor polynucleotide or transposon and a second target polynucleotide, which is also referred to herein as a recipient polynucleotide. As used herein, “transposon” (also referred to as transposable element) refers to a polynucleotide sequence that is capable of moving form location in a genome to another. There are several classes of transposons. Transposons include retrotransposons (Class I transposons) and DNA transposons (Class II transposons). In some cases, retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide.

Any suitable transposon system can be used. Suitable transposon and systems thereof can include, Sleeping Beauty transposon system (Tc1/mariner superfamily) (see e.g. Ivies et al. 1997. Cell. 91(4): 501-510), piggyBac (piggyBac superfamily) (see e.g. Li et al. 2013 110(25): E2279-E2287 and Yusa et al. 2011. PNAS. 108(4): 1531-1536), Tol2 (superfamily hAT), Frog Prince (Tc1/mariner superfamily) (see e.g. Miskey et al. 2003 Nucleic Acid Res. 31(23):6873-6881) and variants thereof.

In some embodiments, the first and/or second Class II transposon polypeptide is a DD[E/D] transposon or transposon polypeptide. In some embodiments, the first and/or the second Class II transposon polynucleotide is a Tc1/mariner, PiggyBac, Frog Prince, Tn3, Tn5, hAT, CACTA, P, Mutator, PIF/Harbinger, Transib, or a Merlin/IS1016 transposon polynucleotide. In some embodiments, the first and/or second Class II transposon polypeptide is a Tc1/mariner, PiggyBac, Frog Prince, Tn3, Tn5, hAT, CACTA, P, Mutator, PIF/Harbinger, Transib, or a Merlin/IS1016 transposon polypeptide.

Suitable Class II transposon systems and components that can be utilized can also be and are not limited to those described in e.g. and without limitation, Han et al., 2013. BMC Genomics. 14:71, doi: 10.1186/1471-2164-14-71, Lopez and Garcia-Perez. 2010. Curr. Genomics. 11(2):115-128; Wessler. 2006. PNAS. 103(47): 176000-17601; Gao et al., 2017. Marine Genomics. 34:67-77; Bradic et al. 2014. Mobile DNA. 5(12) doi:10.1186/1759-8753-5-12; Li et al., 2013. PNAS. 110(25)E2279-E2287; Kebriaei et al. 2017. Trends in Genetics. 33(11): 852-870); Miskey et al. 2003. Nucleic Acid res. 31(23):6873-6881; Nicolas et al. 2015. Microbiol Spectr. 3(4) doi: 10.1128/microbiolspec.MDNA3-0060-2014); W. S. Reznikoff. 1993. Annu Rev. Microbiol. 47:945-963; Rubin et al. 2001. Genetics. 158(3): 949-957; Wicker et al. 2003. Plant Physiol. 132(1): 52-63; Majumdar and Rio. 2015. Microbiol. Spectr. 3(2) doi: 10.1128/microbiolspec.MDNA3-0004-2014; D. Lisch. 2002. Trends in Plant Sci. 7(11): 498-504; Sinzelle et al. 2007. PNAS. 105(12): 4715-4720; Han et al. 2014; Genome Biol. Evol. 6(7):1748-1757; Grzebelus et al. 2006; Mol. Genet. Genomics. 275(5):450-459; Zhang et al. 2004. Genetics. 166(2):971-986; Chen and Li. 2008. Gene. 408(1-2):51-63; and C. Feschotte. 2004. Mol. Biol. Evol. 21(9):1769-1780.

### Polynucleotides and Vectors

The compositions and systems herein may comprise one or more polynucleotides. The polynucleotide(s) may comprise coding sequences of Cas protein(s), guide sequences, or any combination thereof. The present disclosure further provides vectors or vector systems comprising one or more polynucleotides herein. The vectors or vector systems include those described in the delivery sections herein.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. As used herein the term “wild type” is a term of the art understood by skilled persons and means the typical form of an organism, strain, gene or characteristic as it occurs in nature as distinguished from mutant or variant forms. A “wild type” can be a base line. As used herein the term “variant” should be taken to mean the exhibition of qualities that have a pattern that deviates from what occurs in nature. The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature. “Complementarity” refers to the ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y. Where reference is made to a polynucleotide sequence, then complementary or partially complementary sequences are also envisaged. These are preferably capable of hybridizing to the reference sequence under highly stringent conditions. Generally, in order to maximize the hybridization rate, relatively low-stringency hybridization conditions are selected: about 20 to 25° C. lower than the thermal melting point (Tm). The Tm is the temperature at which 50% of specific target sequence hybridizes to a perfectly complementary probe in solution at a defined ionic strength and pH. Generally, in order to require at least about 85% nucleotide complementarity of hybridized sequences, highly stringent washing conditions are selected to be about 5 to 15° C. lower than the Tm. A sequence capable of hybridizing with a given sequence is referred to as the “complement” of the given sequence.

As used herein, the term “genomic locus” or “locus” (plural loci) is the specific location of a gene or DNA sequence on a chromosome. A “gene” refers to stretches of DNA or RNA that encode a polypeptide or an RNA chain that has functional role to play in an organism and hence is the molecular unit of heredity in living organisms. For the purpose of this invention, it may be considered that genes include regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. As used herein, “expression of a genomic locus” or “gene expression” is the process by which information from a gene is used in the synthesis of a functional gene product. The products of gene expression are often proteins, but in non-protein coding genes such as rRNA genes or tRNA genes, the product is functional RNA. The process of gene expression is used by all known life—eukaryotes (including multicellular organisms), prokaryotes (bacteria and archaea) and viruses to generate functional products to survive. As used herein “expression” of a gene or nucleic acid encompasses not only cellular gene expression, but also the transcription and translation of nucleic acid(s) in cloning systems and in any other context. As used herein, “expression” also refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics. As used herein, the term “domain” or “protein domain” refers to a part of a protein sequence that may exist and function independently of the rest of the protein chain. As described in aspects of the present disclosure, sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences.

In certain embodiments, the polynucleotide sequence is recombinant DNA. In further embodiments, the polynucleotide sequence further comprises additional sequences as described elsewhere herein. In certain embodiments, the nucleic acid sequence is synthesized in vitro.

Aspects of the present disclosure relate to polynucleotide molecules that encode one or more components of the CRISPR-Cas system or Cas protein as referred to in any embodiment herein. In certain embodiments, the polynucleotide molecules may comprise further regulatory sequences. By means of guidance and not limitation, the polynucleotide sequence can be part of an expression plasmid, a minicircle, a lentiviral vector, a retroviral vector, an adenoviral or adeno-associated viral vector, a piggyback vector, or a tol2 vector. In certain embodiments, the polynucleotide sequence may be a bicistronic expression construct. In further embodiments, the isolated polynucleotide sequence may be incorporated in a cellular genome. In yet further embodiments, the isolated polynucleotide sequence may be part of a cellular genome. In further embodiments, the isolated polynucleotide sequence may be comprised in an artificial chromosome. In certain embodiments, the 5′ and/or 3′ end of the isolated polynucleotide sequence may be modified to improve the stability of the sequence of actively avoid degradation. In certain embodiments, the isolated polynucleotide sequence may be comprised in a bacteriophage. In other embodiments, the isolated polynucleotide sequence may be contained in Agrobacterium species. In certain embodiments, the isolated polynucleotide sequence is lyophilized.

### Codon Optimization

Aspects of the present disclosure relate to polynucleotide molecules that encode one or more components of one or more CRISPR-Cas systems as described in any of the embodiments herein, wherein at least one or more regions of the polynucleotide molecule may be codon optimized for expression in a eukaryotic cell. In certain embodiments, the polynucleotide molecules that encode one or more components of one or more CRISPR-Cas systems as described in any of the embodiments herein are optimized for expression in a mammalian cell or a plant cell.

An example of a codon optimized sequence, is in this instance a sequence optimized for expression in a eukaryote, e.g., humans (i.e. being optimized for expression in humans), or for another eukaryote, animal or mammal as herein discussed; see, e.g., SaCas9 human codon optimized sequence in International Patent Publication No. WO 2014/093622 (PCT/US2013/074667) as an example of a codon optimized sequence (from knowledge in the art and this disclosure, codon optimizing coding nucleic acid molecule(s), especially as to effector protein is within the ambit of the skilled artisan). Whilst this is preferred, it will be appreciated that other examples are possible and codon optimization for a host species other than human, or for codon optimization for specific organs is known. In some embodiments, an enzyme coding sequence encoding a DNA/RNA-targeting Cas protein is codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence.

Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, P A), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid.

### Delivery

The present disclosure also provides delivery systems for introducing components of the systems and compositions herein to cells, tissues, organs, or organisms. A delivery system may comprise one or more delivery vehicles and/or cargos. Exemplary delivery systems and methods include those described in paragraphs [00117] to [00278] of Feng Zhang et al., (WO2016106236A1), and pages 1241-1251 and Table 1 of Lino C A et al., Delivering CRISPR: a review of the challenges and approaches, DRUG DELIVERY, 2018, VOL. 25, NO. 1, 1234-1257, which are incorporated by reference herein in their entireties.

### Cargos

The delivery systems may comprise one or more cargos. The cargos may comprise one or more components of the systems and compositions herein. A cargo may comprise one or more of the following: i) a plasmid encoding one or more Cas proteins; ii) a plasmid encoding one or more guide RNAs, iii) mRNA of one or more Cas proteins; iv) one or more guide RNAs; v) one or more Cas proteins; vi) any combination thereof. In some examples, a cargo may comprise a plasmid encoding one or more Cas protein and one or more (e.g., a plurality of) guide RNAs. In some embodiments, a cargo may comprise mRNA encoding one or more Cas proteins and one or more guide RNAs.

In some examples, a cargo may comprise one or more Cas proteins and one or more guide RNAs, e.g., in the form of ribonucleoprotein complexes (RNP). The ribonucleoprotein complexes may be delivered by methods and systems herein. In some cases, the ribonucleoprotein may be delivered by way of a polypeptide-based shuttle agent. In one example, the ribonucleoprotein may be delivered using synthetic peptides comprising an endosome leakage domain (ELD) operably linked to a cell penetrating domain (CPD), to a histidine-rich domain and a CPD, e.g., as described in WO2016161516.

### Physical Delivery

In some embodiments, the cargos may be introduced to cells by physical delivery methods. Examples of physical methods include microinjection, electroporation, and hydrodynamic delivery.

### Microinjection

Microinjection of the cargo directly to cells can achieve high efficiency, e.g., above 90% or about 100%. In some embodiments, microinjection may be performed using a microscope and a needle (e.g., with 0.5-5.0 μm in diameter) to pierce a cell membrane and deliver the cargo directly to a target site within the cell. Microinjection may be used for in vitro and ex vivo delivery.

Plasmids comprising coding sequences for Cas proteins and/or guide RNAs, mRNAs, and/or guide RNAs, may be microinjected. In some cases, microinjection may be used i) to deliver DNA directly to a cell nucleus, and/or ii) to deliver mRNA (e.g., in vitro transcribed) to a cell nucleus or cytoplasm. In certain examples, microinjection may be used to delivery sgRNA directly to the nucleus and Cas-encoding mRNA to the cytoplasm, e.g., facilitating translation and shuttling of Cas to the nucleus.

Microinjection may be used to generate genetically modified animals. For example, gene editing cargos may be injected into zygotes to allow for efficient germline modification. Such approach can yield normal embryos and full-term mouse pups harboring the desired modification(s). Microinjection can also be used to provide transiently up- or down-regulate a specific gene within the genome of a cell, e.g., using CRISPRa and CRISPRi.

### Electroporation

In some embodiments, the cargos and/or delivery vehicles may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo into cells. Electroporation may be used for in vitro and ex vivo delivery.

Electroporation may also be used to deliver the cargo to into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391.

### Hydrodynamic Delivery

Hydrodynamic delivery may also be used for delivering the cargos, e.g., for in vivo delivery. In some examples, hydrodynamic delivery may be performed by rapidly pushing a large volume (8-10% body weight) solution containing the gene editing cargo into the bloodstream of a subject (e.g., an animal or human), e.g., for mice, via the tail vein. As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins. The delivered cargos may be enriched in liver, kidney, lung, muscle, and/or heart.

### Transfection

The cargos, e.g., nucleic acids, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid.

### Delivery Vehicles

The delivery systems may comprise one or more delivery vehicles. The delivery vehicles may deliver the cargo into cells, tissues, organs, or organisms (e.g., animals or plants). The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses, non-viral vehicles, and other delivery reagents described herein.

The delivery vehicles in accordance with the present disclosure may a greatest dimension (e.g. diameter) of less than 100 microns (μm). In some embodiments, the delivery vehicles have a greatest dimension of less than 10 μm. In some embodiments, the delivery vehicles may have a greatest dimension of less than 2000 nanometers (nm). In some embodiments, the delivery vehicles may have a greatest dimension of less than 1000 nanometers (nm). In some embodiments, the delivery vehicles may have a greatest dimension (e.g., diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In some embodiments, the delivery vehicles may have a greatest dimension ranging between 25 nm and 200 nm.

In some embodiments, the delivery vehicles may be or comprise particles. For example, the delivery vehicle may be or comprise nanoparticles (e.g., particles with a greatest dimension (e.g., diameter) no greater than 1000 nm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles).

### Vectors

The systems, compositions, and/or delivery systems may comprise one or more vectors. The present disclosure also include vector systems. A vector system may comprise one or more vectors. In some embodiments, a vector refers to a nucleic acid molecule capable of transporting another nucleic acid to which it has been linked. Vectors include nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. A vector may be a plasmid, e.g., a circular double stranded DNA loop into which additional DNA segments can be inserted, such as by standard molecular cloning techniques. Certain vectors may be capable of autonomous replication in a host cell into which they are introduced (e.g., bacterial vectors having a bacterial origin of replication and episomal mammalian vectors). Some vectors (e.g., non-episomal mammalian vectors) are integrated into the genome of a host cell upon introduction into the host cell, and thereby are replicated along with the host genome. In certain examples, vectors may be expression vectors, e.g., capable of directing the expression of genes to which they are operatively-linked. In some cases, the expression vectors may be for expression in eukaryotic cells. Common expression vectors of utility in recombinant DNA techniques are often in the form of plasmids.

Examples of vectors include pGEX, pMAL, pRIT5, E. coli expression vectors (e.g., pTrc, pET 11d, yeast expression vectors (e.g., pYepSec1, pMFa, pJRY88, pYES2, and picZ, Baculovirus vectors (e.g., for expression in insect cells such as SF9 cells) (e.g., pAc series and the pVL series), mammalian expression vectors (e.g., pCDM8 and pMT2PC.

A vector may comprise i) Cas encoding sequence(s), and/or ii) a single, or at least 2, at least 3, at least 4, at least 5, at least 6, at least 7, at least 8, at least 9, at least 10, at least 12, at least 14, at least 16, at least 32, at least 48, at least 50 guide RNA(s) encoding sequences. In a single vector there can be a promoter for each RNA coding sequence. Alternatively, or additionally, in a single vector, there may be a promoter controlling (e.g., driving transcription and/or expression) multiple RNA encoding sequences.

### Regulatory Elements

A vector may comprise one or more regulatory elements. The regulatory element(s) may be operably linked to coding sequences of Cas proteins, accessary proteins, guide RNAs (e.g., a single guide RNA, crRNA, and/or tracrRNA), or combination thereof. The term “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). In certain examples, a vector may comprise: a first regulatory element operably linked to a nucleotide sequence encoding a Cas protein, and a second regulatory element operably linked to a nucleotide sequence encoding a guide RNA.

Examples of regulatory elements include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific.

Examples of promoters include one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter.

### Viral Vectors

The cargos may be delivered by viruses. In some embodiments, viral vectors are used. A viral vector may comprise virally-derived DNA or RNA sequences for packaging into a virus (e.g., retroviruses, replication defective retroviruses, adenoviruses, replication defective adenoviruses, and adeno-associated viruses). Viral vectors also include polynucleotides carried by a virus for transfection into a host cell. Viruses and viral vectors may be used for in vitro, ex vivo, and/or in vivo deliveries.

### Adeno Associated Virus (AAV)

The systems and compositions herein may be delivered by adeno associated virus (AAV). AAV vectors may be used for such delivery. AAV, of the Dependovirus genus and Parvoviridae family, is a single stranded DNA virus. In some embodiments, AAV may provide a persistent source of the provided DNA, as AAV delivered genomic material can exist indefinitely in cells, e.g., either as exogenous DNA or, with some modification, be directly integrated into the host DNA. In some embodiments, AAV do not cause or relate with any diseases in humans. The virus itself is able to efficiently infect cells while provoking little to no innate or adaptive immune response or associated toxicity.

Examples of AAV that can be used herein include AAV-1, AAV-2, AAV-3, AAV-4, AAV-5, AAV-6, AAV-8, and AAV-9. The type of AAV may be selected with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. AAV-2-based vectors were originally proposed for CFTR delivery to CF airways, other serotypes such as AAV-1, AAV-5, AAV-6, and AAV-9 exhibit improved gene transfer efficiency in a variety of models of the lung epithelium. Examples of cell types targeted by AAV are described in Grimm, D. et al, J. Virol. 82: 5887-5911 (2008)), and shown as follows:

TABLE 7

| | AAV- | AAV- | AAV- | AAV- | AAV- | AAV- | AAV- | AAV- |
|---|---|---|---|---|---|---|---|---|
| Cell Line | 1 | 2 | 3 | 4 | 5 | 6 | 8 | 9 |
| Huh-7 | 13 | 100 | 2.5 | 0.0 | 0.1 | 10 | 0.7 | 0.0 |
| HEK293 | 25 | 100 | 2.5 | 0.1 | 0.1 | 5 | 0.7 | 0.1 |
| HeLa | 3 | 100 | 2.0 | 0.1 | 6.7 | 1 | 0.2 | 0.1 |
| HepG2 | 3 | 100 | 16.7 | 0.3 | 1.7 | 5 | 0.3 | ND |
| Hep1A | 20 | 100 | 0.2 | 1.0 | 0.1 | 1 | 0.2 | 0.0 |
| 911 | 17 | 100 | 11 | 0.2 | 0.1 | 17 | 0.1 | ND |
| CHO | 100 | 100 | 14 | 1.4 | 333 | 50 | 10 | 1.0 |
| COS | 33 | 100 | 33 | 3.3 | 5.0 | 14 | 2.0 | 0.5 |
| MeWo | 10 | 100 | 20 | 0.3 | 6.7 | 10 | 1.0 | 0.2 |
| NIH3T3 | 10 | 100 | 2.9 | 2.9 | 0.3 | 10 | 0.3 | ND |
| A549 | 14 | 100 | 20 | ND | 0.5 | 10 | 0.5 | 0.1 |
| HT1180 | 20 | 100 | 10 | 0.1 | 0.3 | 33 | 0.5 | 0.1 |
| Monocytes | 1111 | 100 | ND | ND | 125 | 1429 | ND | ND |
| Immature | 2500 | 100 | ND | ND | 222 | 2857 | ND | ND |
| DC | | | | | | | | |
| Mature DC | 2222 | 100 | ND | ND | 333 | 3333 | ND | ND |

CRISPR-Cas AAV particles may be created in HEK 293 T cells. Once particles with specific tropism have been created, they are used to infect the target cell line much in the same way that native viral particles do. This may allow for persistent presence of CRISPR-Cas components in the infected cell type, and what makes this version of delivery particularly suited to cases where long-term expression is desirable. Examples of doses and formulations for AAV that can be used include those describe in U.S. Pat. Nos. 8,454,972 and 8,404,658.

Various strategies may be used for delivery the systems and compositions herein with AAVs. In some examples, coding sequences of Cas and gRNA may be packaged directly onto one DNA plasmid vector and delivered via one AAV particle. In some examples, AAVs may be used to deliver gRNAs into cells that have been previously engineered to express Cas. In some examples, coding sequences of Cas and gRNA may be made into two separate AAV particles, which are used for co-transfection of target cells. In some examples, markers, tags, and other sequences may be packaged in the same AAV particles as coding sequences of Cas and/or gRNAs.

### Lentiviruses

The systems and compositions herein may be delivered by lentiviruses. Lentiviral vectors may be used for such delivery. Lentiviruses are complex retroviruses that have the ability to infect and express their genes in both mitotic and post-mitotic cells.

Examples of lentiviruses include human immunodeficiency virus (HIV), which may use its envelope glycoproteins of other viruses to target a broad range of cell types; minimal non-primate lentiviral vectors based on the equine infectious anemia virus (EIAV), which may be used for ocular therapies. In certain embodiments, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) may be used/and or adapted to the nucleic acid-targeting system herein.

Lentiviruses may be pseudo-typed with other viral proteins, such as the G protein of vesicular stomatitis virus. In doing so, the cellular tropism of the lentiviruses can be altered to be as broad or narrow as desired. In some cases, to improve safety, second- and third-generation lentiviral systems may split essential genes across three plasmids, which may reduce the likelihood of accidental reconstitution of viable viral particles within cells.

In some examples, leveraging the integration ability, lentiviruses may be used to create libraries of cells comprising various genetic modifications, e.g., for screening and/or studying genes and signaling pathways.

### Adenoviruses

The systems and compositions herein may be delivered by adenoviruses. Adenoviral vectors may be used for such delivery. Adenoviruses include nonenveloped viruses with an icosahedral nucleocapsid containing a double stranded DNA genome. Adenoviruses may infect dividing and non-dividing cells. In some embodiments, adenoviruses do not integrate into the genome of host cells, which may be used for limiting off-target effects of CRISPR-Cas systems in gene editing applications.

### Non-Viral Vehicles

The delivery vehicles may comprise non-viral vehicles. In general, methods and vehicles capable of delivering nucleic acids and/or proteins may be used for delivering the systems compositions herein. Examples of non-viral vehicles include lipid nanoparticles, cell-penetrating peptides (CPPs), DNA nanoclews, gold nanoparticles, streptolysin O, multifunctional envelope-type nanodevices (MENDs), lipid-coated mesoporous silica particles, and other inorganic nanoparticles.

### Lipid Particles

The delivery vehicles may comprise lipid particles, e.g., lipid nanoparticles (LNPs) and liposomes.

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples. LNPs may be used for delivering DNA molecules (e.g., those comprising coding sequences of Cas and/or gRNA) and/or RNA molecules (e.g., mRNA of Cas, gRNAs). In certain cases, LNPs may be use for delivering RNP complexes of Cas/gRNA.

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), R-3-[(ro-methoxy-poly(ethylene glycol)2000) carbamoyl]-1,2-dimyristyloxlpropyl-3-amine (PEG-C-DOMG, and any combination thereof. Preparation of LNPs and encapsulation may be adapted from Rosin et al, Molecular Therapy, vol. 19, no. 12, pages 1286-2200, December 2011).

### Liposomes

In some embodiments, a lipid particle may be liposome. Liposomes are spherical vesicle structures composed of a uni- or multilamellar lipid bilayer surrounding internal aqueous compartments and a relatively impermeable outer lipophilic phospholipid bilayer. In some embodiments, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

### Stable Nucleic-Acid-Lipid Particles (SNALPs)

In some embodiments, the lipid particles may be stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol)2000)carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-cDMA, and 1,2-dilinoleyloxy-3-(N;N-dimethyl)aminopropane (DLinDMA)

### Other Lipids

The lipid particles may also comprise one or more other types of lipids, e.g., cationic lipids, such as amino lipid 2,2-dilinoleyl-4-dimethylaminoethyl[1,3]-dioxolane (DLin-KC2-DMA), DLin-KC2-DMA4, C12-200 and colipids disteroylphosphatidyl choline, cholesterol, and PEG-DMG.

### Lipoplexes/Polyplexes

In some embodiments, the delivery vehicles comprise lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2 [symbol] (e.g., forming DNA/Ca²⁺ microcomplexes), polyethenimine (PEI) (e.g., branched PEI), and poly(L-lysine) (PLL).

### Cell Penetrating Peptides

In some embodiments, the delivery vehicles comprise cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyl). Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the Cas protein directly, which is then complexed with the gRNA and delivered to cells. In some examples, separate delivery of CPP-Cas and CPP-gRNA to multiple cells may be performed. CPP may also be used to delivery RNPs.

### DNA Nanoclews

In some embodiments, the delivery vehicles comprise DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136(42):14722-5; and Sun W et al, Angew Chem Int Ed Engl. 2015 Oct. 5; 54(41):12029-33. DNA nanoclew may have a palindromic sequences to be partially complementary to the gRNA within the Cas:gRNA ribonucleoprotein complex. A DNA nanoclew may be coated, e.g., coated with PEI to induce endosomal escape.

### Gold Nanoparticles

In some embodiments, the delivery vehicles comprise gold nanoparticles (also referred to AuNPs or colloidal gold). Gold nanoparticles may form complex with cargos, e.g., Cas:gRNA RNP. Gold nanoparticles may be coated, e.g., coated in a silicate and an endosomal disruptive polymer, PAsp(DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901.

iTOP

In some embodiments, the delivery vehicles comprise iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D'Astolfo D S, Pagliero R J, Pras A, et al. (2015). Cell 161:674-690.

### Polymer-Based Particles

In some embodiments, the delivery vehicles may comprise polymer-based particles (e.g., nanoparticles). In some embodiments, the polymer-based particles may mimic a viral mechanism of membrane fusion. The polymer-based particles may be a synthetic copy of Influenza virus machinery and form transfection complexes with various types of nucleic acids ((siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In some embodiments, the polymer-based particles may comprise alkylated and carboxyalkylated branched polyethylenimine. In some examples, the polymer-based particles are VIROMER, e.g., VIROMER RNAi, VIROMER RED, VIROMER mRNA, VIROMER CRISPR. Example methods of delivering the systems and compositions herein include those described in Bawage S S et al., Synthetic mRNA expressed Cas13a mitigates RNA virus infections, www.biorxiv.org/content/10.1101/370460v1.full doi: doi.org/10.1101/370460, Viromer® RED, a powerful tool for transfection of keratinocytes. doi: 10.13140/RG.2.2.16993.61281, Viromer® Transfection-Factbook 2018: technology, product overview, users' data., doi:10.13140/RG.2.2.23912.16642.

### Streptolysin O (SLO)

The delivery vehicles may be streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci USA 98:3185-90; Teng K W, et al. (2017). Elife 6:e25460.

### Multifunctional Envelope-Type Nanodevice (MEND)

The delivery vehicles may comprise multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In some embodiments, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles may comprise inorganic nanoparticles. Examples of inorganic nanoparticles include carbon nanotubes (CNTs) (e.g., as described in Bates K and Kostarelos K. (2013). Adv Drug Deliv Rev 65:2023-33.), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Exosomes

The delivery vehicles may comprise exosomes. Exosomes include membrane bound extracellular vesicles, which can be used to contain and delivery various types of biomolecules, such as proteins, carbohydrates, lipids, and nucleic acids, and complexes thereof (e.g., RNPs). Examples of exosomes include those described in Schroeder A, et al., J Intern Med. 2010 January; 267(1):9-21; El-Andaloussi S, et al., Nat Protoc. 2012 December; 7(12):2112-26; Uno Y, et al., Hum Gene Ther. 2011 June; 22(6):711-9; Zou W, et al., Hum Gene Ther. 2011 April; 22(4):465-75.

In some examples, the exosome may form a complex (e.g., by binding directly or indirectly) to one or more components of the cargo. In certain examples, a molecule of an exosome may be fused with first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr. 28. doi: 10.1039/d0bm00427h.

### Genetically Modified Cells and Organisms

The present disclosure further provides cells comprising one or more components of the systems herein, e.g., the Cas protein and/or guide molecule(s). Also provided include cells modified by the systems and methods herein, and cell cultures, tissues, organs, organism comprising such cells or progeny thereof. The present disclosure in some embodiments comprehends a method of modifying an cell or organism. The cell may be a prokaryotic cell or a eukaryotic cell. The cell may be a mammalian cell. The mammalian cell many be a non-human primate, bovine, porcine, rodent or mouse cell. The cell may be a non-mammalian eukaryotic cell such as poultry, fish or shrimp. The cell may be a therapeutic T cell or antibody-producing B-cell. The cell may also be a plant cell. The plant cell may be of a crop plant such as cassava, corn, sorghum, wheat, or rice. The plant cell may also be of an algae, tree or vegetable. The modification introduced to the cell by the present disclosure may be such that the cell and progeny of the cell are altered for improved production of biologic products such as an antibody, starch, alcohol or other desired cellular output. The modification introduced to the cell by the present disclosure may be such that the cell and progeny of the cell include an alteration that changes the biologic product produced.

In some embodiments, one or more polynucleotide molecules, vectors, or vector systems driving expression of one or more elements of a nucleic acid-targeting system or delivery systems comprising one or more elements of the nucleic acid-targeting system are introduced into a host cell such that expression of the elements of the nucleic acid-targeting system direct formation of a nucleic acid-targeting complex at one or more target sites. In certain embodiments of the present disclosure the host cell may be a eukaryotic cell, a prokaryotic cell, or a plant cell.

In particular embodiments, the host cell is a cell of a cell line. Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

Further intended are isolated human cells or tissues, plants or non-human animals comprising one or more of the polynucleotide molecules, vectors, vector systems, or cells described in any of the embodiments herein. In an aspect, host cells and cell lines modified by or comprising the compositions, systems or modified enzymes of present disclosure are provided, including (isolated) stem cells, and progeny thereof.

In certain embodiments, the plants or non-human animals comprise at least one of the CRISPR system components, polynucleotide molecules, vectors, vector systems, or cells described in any of the embodiments herein at least one tissue type of the plant or non-human animal. In certain embodiments, non-human animals comprise at least one of the CRISPR system components, polynucleotide molecules, vectors, vector systems, or cells described in any of the embodiments herein in at least one tissue type. In certain embodiments, the presence of the CRISPR system components is transient, in that they are degraded over time. In certain embodiments, expression of the CRISPR-Cas systems or Cas proteins described in any of the embodiments comprised in polynucleotide molecules, vectors, vector systems, or cells is limited to certain tissue types or regions in the plant or non-human animal. In certain embodiments, the expression of the CRISPR-Cas systems or Cas proteins described in any of the embodiments comprised in polynucleotide molecules, vectors, vector systems, or cells is dependent of a physiological cue. In certain embodiments, expression of the CRISPR-Cas systems or Cas proteins described in any of the embodiments comprised in polynucleotide molecules, vectors, vector systems, or cells may be triggered by an exogenous molecule. In certain embodiments, expression of the CRISPR-Cas systems or Cas proteins described in any of the embodiments comprised in polynucleotide molecules, vectors, vector systems, or cells is dependent on the expression of a non-cas molecule in the plant or non-human animal.

### Methods of Use in General

In another aspect, the present disclosure discloses methods of using the compositions and systems herein. In general, the methods include modifying a target nucleic acid by introducing in a cell or organism that comprises the target nucleic acid the engineered Cas protein, polynucleotide(s) encoding engineered Cas protein, the CRISPR-Cas system, or the vector or vector system comprising the polynucleotide(s), such that the engineered Cas protein modifies the target nucleic acid in the cell or organism. The engineered Cas protein or system may be introduced via delivery by liposomes, nanoparticles, exosomes, microvesicles, nucleic acid nanoassemblies, a gene gun, an implantable device, or the vector system herein. The cell or organisms may be a eukaryotic cell or organism. The cell or organisms is an animal cell or organism. The cell or organisms is a plant cell or organism. Examples of nucleic acid nanoassemblies include DNA origami and RNA origami, e.g., those described in U.S. Pat. No. 8,554,489, US20160103951, WO2017189914, and WO2017189870, which are incorporated by reference in their entireties. A gene gun may include a biolistic particle delivery system, which is a device for delivering exogenous DNA (transgenes) to cells. The payload may be an elemental particle of a heavy metal coated with DNA (typically plasmid DNA). An example of delivery components in CRISPR-Cas systems is described in Svitashev et al., Nat Commun. 2016; 7: 13274.

In some embodiments, the target nucleic acid comprises a genomic locus, and the engineered Cas protein modifies gene product encoded at the genomic locus or expression of the gene product. The target nucleic acid is DNA or RNA and wherein one or more nucleotides in the target nucleic acid may be base edited. The target nucleic acid may be DNA or RNA and wherein the target nucleic acid is cleaved. The engineered Cas protein may further cleave non-target nucleic acid.

In some embodiments, the methods may further comprise visualizing activity and, optionally, using a detectable label. The method may also comprise detecting binding of one or more components of the CRISPR-Cas system to the target nucleic acid.

The aptamer may comprise a polynucleotide-tethered inhibitor that sequesters an enzyme, wherein the enzyme generates a detectable signal upon release from the aptamer or polynucleotide-tethered inhibitor by acting upon a substrate; or may be an inhibitory aptamer that inhibits an enzyme and prevents the enzyme from catalyzing generation of a detectable signal from a substrate or wherein the polynucleotide-tethered inhibitor inhibits an enzyme and prevents the enzyme from catalyzing generation of a detectable signal from a substrate; or sequesters a pair of agents that when released from the aptamers combine to generate a detectable signal.

The nanoparticle may be a colloidal metal. The colloidal metal material may include water-insoluble metal particles or metallic compounds dispersed in a liquid, a hydrosol, or a metal sol. The colloidal metal may be selected from the metals in groups IA, IB, IIB and IIIB of the periodic table, as well as the transition metals, especially those of group VIII. Preferred metals include gold, silver, aluminum, ruthenium, zinc, iron, nickel and calcium. Other suitable metals also include the following in all of their various oxidation states: lithium, sodium, magnesium, potassium, scandium, titanium, vanadium, chromium, manganese, cobalt, copper, gallium, strontium, niobium, molybdenum, palladium, indium, tin, tungsten, rhenium, platinum, and gadolinium. The metals are preferably provided in ionic form, derived from an appropriate metal compound, for example the A1³⁺, Ru³⁺, Zn²⁺, Fe³⁺, Ni²⁺ and Ca²⁺ ions.

When the RNA bridge is cut by the activated CRISPR effector, the before mentioned color shift is observed. In certain example embodiments the particles are colloidal metals. In certain other example embodiments, the colloidal metal is a colloidal gold. In certain example embodiments, the colloidal nanoparticles are 15 nm gold nanoparticles (AuNPs). Due to the unique surface properties of colloidal gold nanoparticles, maximal absorbance is observed at 520 nm when fully dispersed in solution and appear red in color to the naked eye. Upon aggregation of AuNPs, they exhibit a red-shift in maximal absorbance and appear darker in color, eventually precipitating from solution as a dark purple aggregate.

In some embodiments, at least one guide polynucleotide comprises a mismatch. The mismatch may be up- or downstream of a single nucleotide variation on the one or more guide sequences. In certain embodiments, modulations of cleavage efficiency can be exploited by introduction of mismatches, e.g. 1 or more mismatches, such as 1 or 2 mismatches between spacer sequence and target sequence, including the position of the mismatch along the spacer/target. The more central (i.e. not 3′ or 5′) for instance a double mismatch is, the more cleavage efficiency is affected. Accordingly, by choosing mismatch position along the spacer, cleavage efficiency can be modulated. By means of example, if less than 100% cleavage of targets is desired (e.g. in a cell population), 1 or more, such as preferably 2 mismatches between spacer and target sequence may be introduced in the spacer sequences. The more central along the spacer of the mismatch position, the lower the cleavage percentage. In certain example embodiments, the cleavage efficiency may be exploited to design single guides that can distinguish two or more targets that vary by a single nucleotide, such as a single nucleotide polymorphism (SNP), variation, or (point) mutation. The CRISPR effector may have reduced sensitivity to SNPs (or other single nucleotide variations) and continue to cleave SNP targets with a certain level of efficiency. Thus, for two targets, or a set of targets, a guide RNA may be designed with a nucleotide sequence that is complementary to one of the targets i.e. the on-target SNP. The guide RNA is further designed to have a synthetic mismatch. As used herein a “synthetic mismatch” refers to a non-naturally occurring mismatch that is introduced upstream or downstream of the naturally occurring SNP, such as at most 5 nucleotides upstream or downstream, for instance 4, 3, 2, or 1 nucleotide upstream or downstream, preferably at most 3 nucleotides upstream or downstream, more preferably at most 2 nucleotides upstream or downstream, most preferably 1 nucleotide upstream or downstream (i.e. adjacent the SNP). When the CRISPR effector binds to the on-target SNP, only a single mismatch will be formed with the synthetic mismatch and the CRISPR effector will continue to be activated and a detectable signal produced. When the guide RNA hybridizes to an off-target SNP, two mismatches will be formed, the mismatch from the SNP and the synthetic mismatch, and no detectable signal generated. Thus, the systems disclosed herein may be designed to distinguish SNPs within a population. For, example the systems may be used to distinguish pathogenic strains that differ by a single SNP or detect certain disease specific SNPs, such as but not limited to, disease associated SNPs, such as without limitation cancer associated SNPs.

In certain embodiments, the guide RNA is designed such that the SNP is located on position 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the spacer sequence (starting at the 5′ end). In certain embodiments, the guide RNA is designed such that the SNP is located on position 1, 2, 3, 4, 5, 6, 7, 8, or 9 of the spacer sequence (starting at the 5′ end). In certain embodiments, the guide RNA is designed such that the SNP is located on position 2, 3, 4, 5, 6, or 7 of the spacer sequence (starting at the 5′ end). In certain embodiments, the guide RNA is designed such that the SNP is located on position 3, 4, 5, or 6 of the spacer sequence (starting at the 5′ end). In certain embodiments, the guide RNA is designed such that the SNP is located on position 3 of the spacer sequence (starting at the 5′ end).

In certain embodiments, the guide RNA is designed such that the mismatch (e.g. The synthetic mismatch, i.e. an additional mutation besides a SNP) is located on position 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the spacer sequence (starting at the 5′ end). In certain embodiments, the guide RNA is designed such that the mismatch is located on position 1, 2, 3, 4, 5, 6, 7, 8, or 9 of the spacer sequence (starting at the 5′ end). In certain embodiments, the guide RNA is designed such that the mismatch is located on position 4, 5, 6, or 7 of the spacer sequence (starting at the 5′ end. In certain embodiments, the guide RNA is designed such that the mismatch is located on position 5 of the spacer sequence (starting at the 5′ end).

In certain embodiments, the guide RNA is designed such that the mismatch is located 2 nucleotides upstream of the SNP (i.e. one intervening nucleotide). In certain embodiments, the guide RNA is designed such that the mismatch is located 2 nucleotides downstream of the SNP (i.e. one intervening nucleotide). In certain embodiments, the guide RNA is designed such that the mismatch is located on position 5 of the spacer sequence (starting at the 5′ end) and the SNP is located on position 3 of the spacer sequence (starting at the 5′ end).

In an aspect the present disclosure provides a system for specific delivery of functional components to the RNA environment. This can be ensured using the CRISPR systems comprising the Cas proteins of the present disclosure which allow specific targeting of different components to RNA. More particularly such components include activators or repressors, such as activators or repressors of RNA translation, degradation, etc. Applications of this system are described elsewhere herein.

According to one aspect, the present disclosure provides non-naturally occurring or engineered composition comprising a guide RNA comprising a guide sequence capable of hybridizing to a target sequence in a genomic locus of interest in a cell, wherein the guide RNA is modified by the insertion of one or more distinct RNA sequence(s) that bind an adaptor protein. In particular embodiments, the RNA sequences may bind to two or more adaptor proteins (e.g. aptamers), and wherein each adaptor protein is associated with one or more functional domains. The guide RNAs of the CRISPR-Cas enzymes described herein are shown to be amenable to modification of the guide sequence. In particular embodiments, the guide RNA is modified by the insertion of distinct RNA sequence(s) 5′ of the direct repeat, within the direct repeat, or 3′ of the guide sequence. When there is more than one functional domain, the functional domains can be same or different, e.g., two of the same or two different activators or repressors. In an aspect the present disclosure provides a herein-discussed composition, wherein the one or more functional domains are attached to the Cas protein so that upon binding to the target RNA the functional domain is in a spatial orientation allowing for the functional domain to function in its attributed function; In an aspect the present disclosure provides a herein-discussed composition, wherein the composition comprises a CRISPR-Cas complex having at least three functional domains, at least one of which is associated with the Cas protein and at least two of which are associated with the gRNA.

Accordingly, in an aspect, the present disclosure provides non-naturally occurring or engineered CRISPR-Cas complex composition comprising the guide RNA as herein-discussed and a CRISPR-Cas which is an Cas protein, wherein optionally the Cas protein comprises at least one mutation, such that the Cas protein has no more than 5% of the nuclease activity of the enzyme not having the at least one mutation, and optionally one or more comprising at least one or more nuclear localization sequences. In particular embodiments, the guide RNA is additionally or alternatively modified so as to still ensure binding of the Cas protein but to prevent cleavage by the Cas protein (as detailed elsewhere herein).

In particular embodiments, the Cas protein is a Cas protein which has a diminished nuclease activity of at least 97%, or 100% as compared with the CRISPR-Cas enzyme not having the at least one mutation. In an aspect the present disclosure provides a herein-discussed composition, wherein the CRISPR-Cas enzyme comprises two or more mutations as otherwise herein-discussed.

In particular embodiments, a system is provided as described herein above comprising two or more functional domains. In particular embodiments, the two or more functional domains are heterologous functional domain. In particular embodiments, the system comprises an adaptor protein which is a fusion protein comprising a functional domain, the fusion protein optionally comprising a linker between the adaptor protein and the functional domain. In particular embodiments, the linker includes a GlySer linker. Additionally or alternatively, one or more functional domains are attached to the RNA effector protein by way of a linker, optionally a GlySer linker.

In an aspect, the present disclosure provides a herein-discussed composition, wherein the one or more functional domains associated with the adaptor protein or the Cas protein is a domain capable of activating or repressing RNA translation. In an aspect the present disclosure provides a herein-discussed composition, wherein at least one of the one or more functional domains associated with the adaptor protein have one or more activities comprising methylase activity, demethylase activity, transcription activation activity, transcription repression activity, transcription release factor activity, histone modification activity, DNA integration activity RNA cleavage activity, DNA cleavage activity or nucleic acid binding activity, or molecular switch activity or chemical inducibility or light inducibility.

In an aspect, the present disclosure provides a herein-discussed composition comprising an aptamer sequence. In particular embodiments, the aptamer sequence is two or more aptamer sequences specific to the same adaptor protein. In an aspect the present disclosure provides a herein-discussed composition, wherein the aptamer sequence is two or more aptamer sequences specific to different adaptor protein. In an aspect the present disclosure provides a herein-discussed composition, wherein the adaptor protein comprises bacteriophage coat proteins. Accordingly, in particular embodiments, the aptamer is selected from a binding protein specifically binding any one of the adaptor proteins listed above. In an aspect the present disclosure provides a herein-discussed composition, wherein the cell is a eukaryotic cell. In an aspect the present disclosure provides a herein-discussed composition, wherein the eukaryotic cell is a mammalian cell, a plant cell or a yeast cell, whereby the mammalian cell is optionally a mouse cell. In an aspect the present disclosure provides a herein-discussed composition, wherein the mammalian cell is a human cell.

In an aspect, the present disclosure provides a herein above-discussed composition wherein there is more than one guide RNA or gRNA or crRNA, and these target different sequences whereby when the composition is employed, there is multiplexing. In an aspect the present disclosure provides a composition wherein there is more than one guide RNA or gRNA or crRNA modified by the insertion of distinct RNA sequence(s) that bind to one or more adaptor proteins.

In an aspect the present disclosure provides a herein-discussed composition wherein one or more adaptor proteins associated with one or more functional domains is present and bound to the distinct RNA sequence(s) inserted into the guide RNA(s).

In an aspect, the present disclosure provides a herein-discussed composition wherein the guide RNA is modified to have at least one non-coding functional loop; e.g., wherein the at least one non-coding functional loop is repressive; for instance, wherein at least one non-coding functional loop comprises Alu.

In an aspect, the present disclosure provides a method for modifying gene expression comprising the administration to a host or expression in a host in vivo of one or more of the compositions as herein-discussed.

In an aspect, the present disclosure provides a herein-discussed method comprising the delivery of the composition or nucleic acid molecule(s) coding therefor, wherein said nucleic acid molecule(s) are operatively linked to regulatory sequence(s) and expressed in vivo. In an aspect the present disclosure provides a herein-discussed method wherein the expression in vivo is via a lentivirus, an adenovirus, or an AAV.

In an aspect, the present disclosure provides a mammalian cell line of cells as herein-discussed, wherein the cell line is, optionally, a human cell line or a mouse cell line. In an aspect the present disclosure provides a transgenic mammalian model, optionally a mouse, wherein the model has been transformed with a herein-discussed composition or is a progeny of said transformant.

In an aspect, the present disclosure provides a nucleic acid molecule(s) encoding guide RNA or the CRISPR-Cas complex or the composition as herein-discussed. In an aspect the present disclosure provides a vector comprising: a nucleic acid molecule encoding a guide RNA (gRNA) or crRNA comprising a guide sequence capable of hybridizing to an RNA target sequence in a cell, wherein the direct repeat of the gRNA or crRNA is modified by the insertion of distinct RNA sequence(s) that bind(s) to two or more adaptor proteins, and wherein each adaptor protein is associated with one or more functional domains; or, wherein the gRNA is modified to have at least one non-coding functional loop. In an aspect, the present disclosure provides vector(s) comprising nucleic acid molecule(s) encoding: non-naturally occurring or engineered CRISPR-Cas complex composition comprising the gRNA or crRNA herein-discussed, and an Cas protein, wherein optionally the Cas protein comprises at least one mutation, such that the Cas protein has no more than 5% of the nuclease activity of the Cas protein not having the at least one mutation, and optionally one or more comprising at least one or more nuclear localization sequences. In an aspect, a vector can further comprise regulatory element(s) operable in a eukaryotic cell operably linked to the nucleic acid molecule encoding the guide RNA (gRNA) or crRNA and/or the nucleic acid molecule encoding the Cas protein and/or the optional nuclear localization sequence(s).

In one aspect, the present disclosure provides a kit comprising one or more of the components described herein. In some embodiments, the kit comprises a vector system as described herein and instructions for using the kit.

In an aspect, the present disclosure provides a method of screening for gain of function (GOF) or loss of function (LOF) or for screening non-coding RNAs or potential regulatory regions (e.g. enhancers, repressors) comprising the cell line of as herein-discussed or cells of the model herein-discussed containing or expressing the Cas protein and introducing a composition as herein-discussed into cells of the cell line or model, whereby the gRNA or crRNA includes either an activator or a repressor, and monitoring for GOF or LOF respectively as to those cells as to which the introduced gRNA or crRNA includes an activator or as to those cells as to which the introduced gRNA or crRNA includes a repressor.

In an aspect, the present disclosure provides a library of non-naturally occurring or engineered compositions, each comprising a CRISPR guide RNA (gRNA) or crRNA comprising a guide sequence capable of hybridizing to a target RNA sequence of interest in a cell, an Cas protein, wherein the Cas protein comprises at least one mutation, such that the Cas protein has no more than 5% of the nuclease activity of the Cas protein not having the at least one mutation, wherein the gRNA or crRNA is modified by the insertion of distinct RNA sequence(s) that bind to one or more adaptor proteins, and wherein the adaptor protein is associated with one or more functional domains, wherein the composition comprises one or more or two or more adaptor proteins, wherein the each protein is associated with one or more functional domains, and wherein the gRNAs or crRNAs comprise a genome wide library comprising a plurality of guide RNAs (gRNAs) or crRNAs. In an aspect, the present disclosure provides a library as herein-discussed, wherein the Cas protein has a diminished nuclease activity of at least 97%, or 100% as compare with the Cas protein not having the at least one mutation. In an aspect, the present disclosure provides a library as herein-discussed, wherein the adaptor protein is a fusion protein comprising the functional domain. In an aspect the present disclosure provides a library as herein discussed, wherein the gRNA or crRNA is not modified by the insertion of distinct RNA sequence(s) that bind to the one or two or more adaptor proteins. In an aspect, the present disclosure provides a library as herein discussed, wherein the one or two or more functional domains are associated with the Cas protein. In an aspect, the present disclosure provides a library as herein discussed, wherein the cell population of cells is a population of eukaryotic cells. In an aspect, the present disclosure provides a library as herein discussed, wherein the eukaryotic cell is a mammalian cell, a plant cell or a yeast cell. In an aspect, the present disclosure provides a library as herein discussed, wherein the mammalian cell is a human cell. In an aspect, the present disclosure provides a library as herein discussed, wherein the population of cells is a population of embryonic stem (ES) cells.

In an aspect, the present disclosure provides a library as herein discussed, wherein the targeting is of about 100 or more RNA sequences. In an aspect the present disclosure provides a library as herein discussed, wherein the targeting is of about 1000 or more RNA sequences. In an aspect the present disclosure provides a library as herein discussed, wherein the targeting is of about 20,000 or more sequences. In an aspect the present disclosure provides a library as herein discussed, wherein the targeting is of the entire transcriptome. In an aspect the present disclosure provides a library as herein discussed, wherein the targeting is of a panel of target sequences focused on a relevant or desirable pathway. In an aspect the present disclosure provides a library as herein discussed, wherein the pathway is an immune pathway. In an aspect the present disclosure provides a library as herein discussed, wherein the pathway is a cell division pathway.

In one aspect, the present disclosure provides a method of generating a model eukaryotic cell comprising a gene with modified expression. In some embodiments, a disease gene is any gene associated an increase in the risk of having or developing a disease. In some embodiments, the method comprises (a) introducing one or more vectors encoding the components of the system described herein above into a eukaryotic cell, and (b) allowing a CRISPR complex to bind to a target polynucleotide so as to modify expression of a gene, thereby generating a model eukaryotic cell comprising modified gene expression.

The structural information provided herein allows for interrogation of guide RNA or crRNA interaction with the target RNA and the Cas protein permitting engineering or alteration of guide RNA structure to optimize functionality of the entire CRISPR-Cas system. For example, the guide RNA or crRNA may be extended, without colliding with the Cas protein by the insertion of adaptor proteins that can bind to RNA. These adaptor proteins can further recruit effector proteins or fusions which comprise one or more functional domains.

An aspect of the present disclosure is that the above elements are comprised in a single composition or comprised in individual compositions. These compositions may advantageously be applied to a host to elicit a functional effect on the genomic level.

The skilled person will understand that modifications to the guide RNA or crRNA which allow for binding of the adapter+functional domain but not proper positioning of the adapter+functional domain (e.g. due to steric hindrance within the three dimension structure of the CRISPR-Cas complex) are modifications which are not intended. The one or more modified guide RNA or crRNA may be modified, by introduction of a distinct RNA sequence(s) 5′ of the direct repeat, within the direct repeat, or 3′ of the guide sequence.

The modified guide RNA or crRNA, the inactivated Cas protein (with or without functional domains), and the binding protein with one or more functional domains, may each individually be comprised in a composition and administered to a host individually or collectively. Alternatively, these components may be provided in a single composition for administration to a host. Administration to a host may be performed via viral vectors known to the skilled person or described herein for delivery to a host (e.g. lentiviral vector, adenoviral vector, AAV vector). As explained herein, use of different selection markers (e.g. for lentiviral gRNA or crRNA selection) and concentration of gRNA or crRNA (e.g. dependent on whether multiple gRNAs or crRNAs are used) may be advantageous for eliciting an improved effect.

Using the provided compositions, the person skilled in the art can advantageously and specifically target single or multiple loci with the same or different functional domains to elicit one or more genomic events. The compositions may be applied in a wide variety of methods for screening in libraries in cells and functional modeling in vivo (e.g. gene activation of lincRNA and identification of function; gain-of-function modeling; loss-of-function modeling; the use the compositions of the present disclosure to establish cell lines and transgenic animals for optimization and screening purposes).

The current present disclosure comprehends the use of the compositions of the current present disclosure to establish and utilize conditional or inducible CRISPR-Cas events. (See, e.g., Platt et al., Cell (2014), dx.doi.org/10.1016/j.cell.2014.09.014, or PCT patent publications cited herein, such as WO 2014/093622 (PCT/US2013/074667), which are not believed prior to the present disclosure or application).

### Transcript Tracking

In another aspect, the present disclosure provides compositions and methods for transcript tracking. In some embodiments, transcript tracking allows researchers to visualize transcripts in cells, tissues, organs or animals, providing important spatio-temporal information regarding RNA dynamics and function.

In some embodiments, the compositions may be a Cas protein herein with one or more labels, or a CRISPR-Cas system comprising such labeled Cas protein. The Cas protein or system may bind to one or more transcripts such that the transcripts may be detected (e.g., visualized) using the label on the Cas protein.

In some embodiments, the present disclosure includes a system for expressing a Cas protein with one or more polypeptides or polynucleotide labels. The system may comprise polynucleotides encoding the Cas protein and/or the labels. The system may further include vector systems comprising such polynucleotides. For example, a Cas protein may be fused with a fluorescent protein or a fragment thereof. Examples of fluorescent proteins include GFP proteins, EGFP, Azami-Green, Kaede, ZsGreen1 and CopGFP; CFP proteins, such as Cerulean, mCFP, AmCyan1, MiCy, and CyPet; BFP proteins such as EBFP; YFP proteins such as EYFP, YPet, Venus, ZsYellow, and mCitrine; OFP proteins such as cOFP, mKO, and mOrange; red fluorescent protein, or RFP; red or far-red fluorescent proteins from any other species, such as Heteractis reef coral and Actinia or Entacmaea sea anemone, as well as variants thereof. RFPs include, for example, Discosoma variants, such as mRFP1, mCherry, tdTomato, mStrawberry, mTangerine, DsRed2, and DsRed-T1, Anthomedusa J-Red and Anemonia AsRed2. Far-red fluorescent proteins include, for example, Actinia AQ143, Entacmaea eqFP611, Discosoma variants such as mPlum and mRasberry, and Heteractis HcRed1 and t-HcRed.

In some cases, the systems for expressing the labeled Cas protein may be inducible. For example, the systems may comprise polynucleotides encoding the Cas protein and/or labels under control of a regulatory element herein, e.g., inducible promoters. Such systems may allow spatial and/or temporal control of the expression of the labels, thus enabling spatial and/or temporal control of transcript tracking.

In certain cases, the CRISPR-Cas may be labeled with a detectable tag. The labeling may be performed in cells. Alternatively or additionally, the labeling may be performed first and the labeled Cas protein is then delivered into cells, tissues, organs, or organs.

The detectable tags may be detected (e.g., visualized by imaging, ultrasound, or MRI). Examples of such detectable tags include detectable oligonucleotide tags may be, but are not limited to, oligonucleotides comprising unique nucleotide sequences, oligonucleotides comprising detectable moieties, and oligonucleotides comprising both unique nucleotide sequences and detectable moieties. In some cases, the detectable tag comprises a labeling substance, which is detectable by spectroscopic, photochemical, biochemical, immunochemical, electrical, optical or chemical means. Such tags include biotin for staining with labeled streptavidin conjugate, magnetic beads (e.g., Dynabeads®), fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Detectable tags may be detected by many methods. For example, radiolabels may be detected using photographic film or scintillation counters, fluorescent markers may be detected using a photodetector to detect emitted light. Enzymatic labels are typically detected by providing the enzyme with a substrate and detecting, the reaction product produced by the action of the enzyme on the substrate, and calorimetric labels are detected by simply visualizing the colored label. Examples of the labeling substance which may be employed include labeling substances known to those skilled in the art, such as fluorescent dyes, enzymes, coenzymes, chemiluminescent substances, and radioactive substances. Specific examples include radioisotopes (e.g., ³²P, ¹⁴C, ¹²⁵I, ¹³H, and ¹³¹I) fluorescein, rhodamine, dansyl chloride, umbelliferone, luciferase, peroxidase, alkaline phosphatase, β-galactosidase, β-glucosidase, horseradish peroxidase, glucoamylase, lysozyme, saccharide oxidase, microperoxidase, biotin, and ruthenium. In the case where biotin is employed as a labeling substance, preferably, after addition of a biotin-labeled antibody, streptavidin bound to an enzyme (e.g., peroxidase) is further added. Advantageously, the label is a fluorescent label. Examples of fluorescent labels include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 di sulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine. A fluorescent label may be a fluorescent protein, such as blue fluorescent protein, cyan fluorescent protein, green fluorescent protein, red fluorescent protein, yellow fluorescent protein or any photoconvertible protein. Colorimetric labeling, bioluminescent labeling and/or chemiluminescent labeling may further accomplish labeling. Labeling further may include energy transfer between molecules in the hybridization complex by perturbation analysis, quenching, or electron transport between donor and acceptor molecules, the latter of which may be facilitated by double stranded match hybridization complexes. The fluorescent label may be a perylene or a terrylen. In the alternative, the fluorescent label may be a fluorescent bar code. Advantageously, the label may be light sensitive, wherein the label is light-activated and/or light cleaves the one or more linkers to release the molecular cargo. The light-activated molecular cargo may be a major light-harvesting complex (LHCII). In another embodiment, the fluorescent label may induce free radical formation. In some embodiments, the detectable moieties may be quantum dots.

In some embodiments, the present disclosure provides for a system for delivery the labeled Cas proteins or labeled CRISPR-Cas systems. The delivery system may comprise any delivery vehicles, e.g., those described herein such as RNP, liposomes, nanoparticles, exosomes, microvesicles, nucleic acid nanoassemblies, a gene gun, an implantable device, or the vector systems herein.

### Nucleic Acid Targeting

In certain embodiments, the CRISPR-Cas protein of the present disclosure is, or in, or comprises, or consists essentially of, or consists of, or involves or relates to such a protein from or as set forth in Tables 1-5, wherein one or more amino acids are mutated, as described herein elsewhere. Thus, in some embodiments, the effector protein may be a RNA-binding protein, such as a dead-Cas type effector protein, which may be optionally functionalized as described herein for instance with an transcriptional activator or repressor domain, NLS or other functional domain. In some embodiments, the effector protein may be a RNA-binding protein that cleaves a single strand of RNA. If the RNA bound is ssRNA, then the ssRNA is fully cleaved. In some embodiments, the effector protein may be a RNA-binding protein that cleaves a double strand of RNA, for example if it comprises two RNase domains. If the RNA bound is dsRNA, then the dsRNA is fully cleaved. In some embodiments, the effector protein may be a RNA-binding protein that has nickase activity, i.e. it binds dsRNA, but only cleaves one of the RNA strands.

RNase function in CRISPR systems is known, for example mRNA targeting has been reported for certain type III CRISPR-Cas systems (Hale et al., 2014, Genes Dev, vol. 28, 2432-2443; Hale et al., 2009, Cell, vol. 139, 945-956; Peng et al., 2015, Nucleic acids research, vol. 43, 406-417) and provides significant advantages. A CRISPR-Cas system, composition or method targeting RNA via the present effector proteins is thus provided.

The target RNA, i.e. the RNA of interest, is the RNA to be targeted by the present disclosure leading to the recruitment to, and the binding of the effector protein at, the target site of interest on the target RNA. The target RNA may be any suitable form of RNA. This may include, in some embodiments, mRNA. In other embodiments, the target RNA may include tRNA or rRNA.

### DNA Cleavage and Repair

The method comprises modifying a target polynucleotide using a CRISPR complex that binds to the target polynucleotide and effect cleavage of said target polynucleotide. Typically, the CRISPR complex of the present disclosure, when introduced into a cell, creates a break (e.g., a single or a double strand break) in the genome sequence. For example, the method can be used to cleave a disease gene in a cell. The break created by the CRISPR complex can be repaired by a repair processes such as the error prone non-homologous end joining (NHEJ) pathway or the high fidelity homology-directed repair (HDR). During these repair process, an exogenous polynucleotide template can be introduced into the genome sequence. In some methods, the HDR process is used modify genome sequence. For example, an exogenous polynucleotide template comprising a sequence to be integrated flanked by an upstream sequence and a downstream sequence is introduced into a cell. The upstream and downstream sequences share sequence similarity with either side of the site of integration in the chromosome. Where desired, a donor polynucleotide can be DNA, e.g., a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function. The upstream and downstream sequences in the exogenous polynucleotide template are selected to promote recombination between the chromosomal sequence of interest and the donor polynucleotide. The upstream sequence is a nucleic acid sequence that shares sequence similarity with the genome sequence upstream of the targeted site for integration. Similarly, the downstream sequence is a nucleic acid sequence that shares sequence similarity with the chromosomal sequence downstream of the targeted site of integration. The upstream and downstream sequences in the exogenous polynucleotide template can have 75%, 80%, 85%, 90%, 95%, or 100% sequence identity with the targeted genome sequence. Preferably, the upstream and downstream sequences in the exogenous polynucleotide template have about 95%, 96%, 97%, 98%, 99%, or 100% sequence identity with the targeted genome sequence. In some methods, the upstream and downstream sequences in the exogenous polynucleotide template have about 99% or 100% sequence identity with the targeted genome sequence. An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000 bp. In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the present disclosure can be constructed using recombinant techniques (see, for example, Sambrook et al., 2001 and Ausubel et al., 1996). In a method for modifying a target polynucleotide by integrating an exogenous polynucleotide template, a double stranded break is introduced into the genome sequence by the CRISPR complex, the break is repaired via homologous recombination an exogenous polynucleotide template such that the template is integrated into the genome. The presence of a double-stranded break facilitates integration of the template. In other embodiments, this present disclosure provides a method of modifying expression of a polynucleotide in a eukaryotic cell. The method comprises increasing or decreasing expression of a target polynucleotide by using a CRISPR complex that binds to the polynucleotide. In some methods, a target polynucleotide can be inactivated to effect the modification of the expression in a cell. For example, upon the binding of a CRISPR complex to a target sequence in a cell, the target polynucleotide is inactivated such that the sequence is not transcribed, the coded protein is not produced, or the sequence does not function as the wild-type sequence does. For example, a protein or microRNA coding sequence may be inactivated such that the protein or microRNA or pre-microRNA transcript is not produced. In some methods, a control sequence can be inactivated such that it no longer functions as a control sequence. As used herein, “control sequence” refers to any nucleic acid sequence that effects the transcription, translation, or accessibility of a nucleic acid sequence. Examples of a control sequence include, a promoter, a transcription terminator, and an enhancer are control sequences. The target polynucleotide of a CRISPR complex can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide or a junk DNA). Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level. The target polynucleotide of a CRISPR complex can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide or a junk DNA).

Gene Editing or Altering a Target Loci with Cas9; HDR and Templates

The double strand break or single strand break in one of the strands advantageously should be sufficiently close to target position such that correction occurs. In an embodiment, the distance is not more than 50, 100, 200, 300, 350 or 400 nucleotides. While not wishing to be bound by theory, it is believed that the break should be sufficiently close to target position such that the break is within the region that is subject to exonuclease-mediated removal during end resection. If the distance between the target position and a break is too great, the mutation may not be included in the end resection and, therefore, may not be corrected, as the template nucleic acid sequence may only be used to correct sequence within the end resection region.

In an embodiment, in which a guide RNA and a Type II molecule, in particular Cas9 or an ortholog or homolog thereof, preferably a Cas9 nuclease induce a double strand break for the purpose of inducing HDR-mediated correction, the cleavage site is between 0-200 bp (e.g., 0 to 175, 0 to 150, 0 to 125, 0 to 100, 0 to 75, 0 to 50, 0 to 25, 25 to 200, 25 to 175, 25 to 150, 25 to 125, 25 to 100, 25 to 75, 25 to 50, 50 to 200, 50 to 175, 50 to 150, 50 to 125, 50 to 100, 50 to 75, 75 to 200, 75 to 175, 75 to 150, 75 to 125, 75 to 100 bp) away from the target position. In an embodiment, the cleavage site is between 0-100 bp (e.g., 0 to 75, 0 to 50, 0 to 25, 25 to 100, 25 to 75, 25 to 50, 50 to 100, 50 to 75 or 75 to 100 bp) away from the target position. In a further embodiment, two or more guide RNAs complexing with Cas9 or an ortholog or homolog thereof, may be used to induce multiplexed breaks for purpose of inducing HDR-mediated correction.

The homology arm should extend at least as far as the region in which end resection may occur, e.g., in order to allow the resected single stranded overhang to find a complementary region within the donor template. The overall length could be limited by parameters such as plasmid size or viral packaging limits. In an embodiment, a homology arm may not extend into repeated elements. Exemplary homology arm lengths include a least 50, 100, 250, 500, 750 or 1000 nucleotides.

Target position, as used herein, refers to a site on a target nucleic acid or target gene (e.g., the chromosome) that is modified by a Type II, in particular Cas9 or an ortholog or homolog thereof, preferably Cas9 molecule-dependent process. For example, the target position can be a modified Cas9 molecule cleavage of the target nucleic acid and template nucleic acid directed modification, e.g., correction, of the target position. In an embodiment, a target position can be a site between two nucleotides, e.g., adjacent nucleotides, on the target nucleic acid into which one or more nucleotides is added. The target position may comprise one or more nucleotides that are altered, e.g., corrected, by a template nucleic acid. In an embodiment, the target position is within a target sequence (e.g., the sequence to which the guide RNA binds). In an embodiment, a target position is upstream or downstream of a target sequence (e.g., the sequence to which the guide RNA binds).

A template nucleic acid, as that term is used herein, refers to a nucleic acid sequence which can be used in conjunction with a Type II molecule, in particular Cas9 or an ortholog or homolog thereof, preferably a Cas9 molecule and a guide RNA molecule to alter the structure of a target position. In an embodiment, the target nucleic acid is modified to have some or all of the sequence of the template nucleic acid, typically at or near cleavage site(s). In an embodiment, the template nucleic acid is single stranded. In an alternate embodiment, the template nucleic acid is double stranded. In an embodiment, the template nucleic acid is DNA, e.g., double stranded DNA. In an alternate embodiment, the template nucleic acid is single stranded DNA.

In an embodiment, the template nucleic acid alters the structure of the target position by participating in homologous recombination. In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include sequence that corresponds to a site on the target sequence that is cleaved by a Cas9 mediated cleavage event. In an embodiment, the template nucleic acid may include sequence that corresponds to both, a first site on the target sequence that is cleaved in a first Cas9 mediated event, and a second site on the target sequence that is cleaved in a second Cas9 mediated event.

In certain embodiments, the template nucleic acid can include sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include sequence which, when integrated, results in: decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include sequence which results in: a change in sequence of 1, 2, 3, 4, 5, 6, 7, 8, 9, 10, 11, 12 or more nucleotides of the target sequence. In an embodiment, the template nucleic acid may be 20+/−10, 30+/−10, 40+/−10, 50+/−10, 60+/−10, 70+/−10, 80+/−10, 90+/−10, 100+/−10, 110+/−10, 120+/−10, 130+/−10, 140+/−10, 150+/−10, 160+/−10, 170+/−10, 180+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, I 50+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

A template nucleic acid comprises the following components: [5′ homology arm]-[replacement sequence]-[3′ homology arm]. The homology arms provide for recombination into the chromosome, thus replacing the undesired element, e.g., a mutation or signature, with the replacement sequence. In an embodiment, the homology arms flank the most distal cleavage sites. In an embodiment, the 3′ end of the 5′ homology arm is the position next to the 5′ end of the replacement sequence. In an embodiment, the 5′ homology arm can extend at least 10, 20, 30, 40, 50, 100, 200, 300, 400, 500, 600, 700, 800, 900, 1000, 1500, or 2000 nucleotides 5′ from the 5′ end of the replacement sequence. In an embodiment, the 5′ end of the 3′ homology arm is the position next to the 3′ end of the replacement sequence. In an embodiment, the 3′ homology arm can extend at least 10, 20, 30, 40, 50, 100, 200, 300, 400, 500, 600, 700, 800, 900, 1000, 1500, or 2000 nucleotides 3′ from the 3′ end of the replacement sequence.

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In certain embodiments, a template of nucleic acids for correcting a mutation may designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

### DNA Repair and NHEJ

In certain embodiments, nuclease-induced non-homologous end-joining (NHEJ) can be used to target gene-specific knockouts. Nuclease-induced NHEJ can also be used to remove (e.g., delete) sequence in a gene of interest. Generally, NHEJ repairs a double-strand break in the DNA by joining together the two ends; however, generally, the original sequence is restored only if two compatible ends, exactly as they were formed by the double-strand break, are perfectly ligated. The DNA ends of the double-strand break are frequently the subject of enzymatic processing, resulting in the addition or removal of nucleotides, at one or both strands, prior to rejoining of the ends. This results in the presence of insertion and/or deletion (indel) mutations in the DNA sequence at the site of the NHEJ repair. Two-thirds of these mutations typically alter the reading frame and, therefore, produce a non-functional protein. Additionally, mutations that maintain the reading frame, but which insert or delete a significant amount of sequence, can destroy functionality of the protein. This is locus dependent as mutations in critical functional domains are likely less tolerable than mutations in non-critical regions of the protein. The indel mutations generated by NHEJ are unpredictable in nature; however, at a given break site certain indel sequences are favored and are over represented in the population, likely due to small regions of microhomology. The lengths of deletions can vary widely; most commonly in the 1-50 bp range, but they can easily be greater than 50 bp, e.g., they can easily reach greater than about 100-200 bp. Insertions tend to be shorter and often include short duplications of the sequence immediately surrounding the break site. However, it is possible to obtain large insertions, and in these cases, the inserted sequence has often been traced to other regions of the genome or to plasmid DNA present in the cells.

Because NHEJ is a mutagenic process, it may also be used to delete small sequence motifs as long as the generation of a specific final sequence is not required. If a double-strand break is targeted near to a short target sequence, the deletion mutations caused by the NHEJ repair often span, and therefore remove, the unwanted nucleotides. For the deletion of larger DNA segments, introducing two double-strand breaks, one on each side of the sequence, can result in NHEJ between the ends with removal of the entire intervening sequence. Both of these approaches can be used to delete specific DNA sequences; however, the error-prone nature of NHEJ may still produce indel mutations at the site of repair.

Both double strand cleaving Type II molecule, in particular Cas9 or an ortholog or homolog thereof, preferably Cas9 molecules and single strand, or nickase, Type II molecule, in particular Cas9 or an ortholog or homolog thereof, preferably Cas9 molecules can be used in the methods and compositions described herein to generate NHEJ-mediated indels. NHEJ-mediated indels targeted to the gene, e.g., a coding region, e.g., an early coding region of a gene of interest can be used to knockout (i.e., eliminate expression of) a gene of interest. For example, early coding region of a gene of interest includes sequence immediately following a transcription start site, within a first exon of the coding sequence, or within 500 bp of the transcription start site (e.g., less than 500, 450, 400, 350, 300, 250, 200, 150, 100 or 50 bp).

In an embodiment, in which a guide RNA and Type II molecule, in particular Cas9 or an ortholog or homolog thereof, preferably Cas9 nuclease generate a double strand break for the purpose of inducing NHEJ-mediated indels, a guide RNA may be configured to position one double-strand break in close proximity to a nucleotide of the target position. In an embodiment, the cleavage site may be between 0-500 bp away from the target position (e.g., less than 500, 400, 300, 200, 100, 50, 40, 30, 25, 20, 15, 10, 9, 8, 7, 6, 5, 4, 3, 2 or 1 bp from the target position).

In an embodiment, in which two guide RNAs complexing with Type II molecules, in particular Cas9 or an ortholog or homolog thereof, preferably Cas9 nickases induce two single strand breaks for the purpose of inducing NHEJ-mediated indels, two guide RNAs may be configured to position two single-strand breaks to provide for NHEJ repair a nucleotide of the target position.

### Self-Inactivating Systems

Once all copies of RNA in a cell have been edited, continued a CRISPR-Cas protein expression or activity in that cell is no longer necessary. A Self-Inactivating system that relies on the use of RNA as to the CRISPR-Cas or crRNA as the guide target sequence can shut down the system by preventing expression of CRISPR-Cas or complex formation.

### Modulation of Cellular Status

In certain embodiments CRISPR-Cas in a complex with crRNA is activated upon binding to target RNA and subsequently cleaves any nearby ssRNA targets (i.e. “collateral” or “bystander” effects). CRISPR-Cas, once primed by the cognate target, can cleave other (non-complementary) RNA molecules. Such promiscuous RNA cleavage could potentially cause cellular toxicity, or otherwise affect cellular physiology or cell status.

Accordingly, in certain embodiments, the non-naturally occurring or engineered composition, vector system, or delivery systems as described herein are used for or are for use in induction of cell dormancy. In certain embodiments, the non-naturally occurring or engineered composition, vector system, or delivery systems as described herein are used for or are for use in induction of cell cycle arrest. In certain embodiments, the non-naturally occurring or engineered composition, vector system, or delivery systems as described herein are used for or are for use in reduction of cell growth and/or cell proliferation, In certain embodiments, the non-naturally occurring or engineered composition, vector system, or delivery systems as described herein are used for or are for use in induction of cell anergy. In certain embodiments, the non-naturally occurring or engineered composition, vector system, or delivery systems as described herein are used for or are for use in induction of cell apoptosis. In certain embodiments, the non-naturally occurring or engineered composition, vector system, or delivery systems as described herein are used for or are for use in induction of cell necrosis. In certain embodiments, the non-naturally occurring or engineered composition, vector system, or delivery systems as described herein are used for or are for use in induction of cell death. In certain embodiments, the non-naturally occurring or engineered composition, vector system, or delivery systems as described herein are used for or are for use in induction of programmed cell death.

In certain embodiments, the present disclosure relates to a method for induction of cell dormancy comprising introducing or inducing the non-naturally occurring or engineered composition, vector system, or delivery systems as described herein. In certain embodiments, the present disclosure relates to a method for induction of cell cycle arrest comprising introducing or inducing the non-naturally occurring or engineered composition, vector system, or delivery systems as described herein. In certain embodiments, the present disclosure relates to a method for reduction of cell growth and/or cell proliferation comprising introducing or inducing the non-naturally occurring or engineered composition, vector system, or delivery systems as described herein. In certain embodiments, the present disclosure relates to a method for induction of cell anergy comprising introducing or inducing the non-naturally occurring or engineered composition, vector system, or delivery systems as described herein. In certain embodiments, the present disclosure relates to a method for induction of cell apoptosis comprising introducing or inducing the non-naturally occurring or engineered composition, vector system, or delivery systems as described herein. In certain embodiments, the present disclosure relates to a method for induction of cell necrosis comprising introducing or inducing the non-naturally occurring or engineered composition, vector system, or delivery systems as described herein. In certain embodiments, the present disclosure relates to a method for induction of cell death comprising introducing or inducing the non-naturally occurring or engineered composition, vector system, or delivery systems as described herein. In certain embodiments, the present disclosure relates to a method for induction of programmed cell death comprising introducing or inducing the non-naturally occurring or engineered composition, vector system, or delivery systems as described herein.

The methods and uses as described herein may be therapeutic or prophylactic and may target particular cells, cell (sub)populations, or cell/tissue types. In particular, the methods and uses as described herein may be therapeutic or prophylactic and may target particular cells, cell (sub)populations, or cell/tissue types expressing one or more target sequences, such as one or more particular target RNA (e.g. ssRNA). Without limitation, target cells may for instance be cancer cells expressing a particular transcript, e.g. neurons of a given class, (immune) cells causing e.g. autoimmunity, or cells infected by a specific (e.g. viral) pathogen, etc.

Accordingly, in certain embodiments, the present disclosure relates to a method for treating a pathological condition characterized by the presence of undesirable cells (host cells), comprising introducing or inducing the non-naturally occurring or engineered composition, vector system, or delivery systems as described herein. In certain embodiments, the present disclosure relates the use of the non-naturally occurring or engineered composition, vector system, or delivery systems as described herein for treating a pathological condition characterized by the presence of undesirable cells (host cells). In certain embodiments, the present disclosure relates the non-naturally occurring or engineered composition, vector system, or delivery systems as described herein for use in treating a pathological condition characterized by the presence of undesirable cells (host cells). It is to be understood that preferably the CRISPR-Cas system targets a target specific for the undesirable cells. In certain embodiments, the present disclosure relates to the use of the non-naturally occurring or engineered composition, vector system, or delivery systems as described herein for treating, preventing, or alleviating cancer. In certain embodiments, the present disclosure relates to the non-naturally occurring or engineered composition, vector system, or delivery systems as described herein for use in treating, preventing, or alleviating cancer. In certain embodiments, the present disclosure relates to a method for treating, preventing, or alleviating cancer comprising introducing or inducing the non-naturally occurring or engineered composition, vector system, or delivery systems as described herein. It is to be understood that preferably the CRISPR-Cas system targets a target specific for the cancer cells. In certain embodiments, the present disclosure relates to the use of the non-naturally occurring or engineered composition, vector system, or delivery systems as described herein for treating, preventing, or alleviating infection of cells by a pathogen. In certain embodiments, the present disclosure relates to the non-naturally occurring or engineered composition, vector system, or delivery systems as described herein for use in treating, preventing, or alleviating infection of cells by a pathogen. In certain embodiments, the present disclosure relates to a method for treating, preventing, or alleviating infection of cells by a pathogen comprising introducing or inducing the non-naturally occurring or engineered composition, vector system, or delivery systems as described herein. It is to be understood that preferably the CRISPR-Cas system targets a target specific for the cells infected by the pathogen (e.g. a pathogen derived target). In certain embodiments, the present disclosure relates to the use of the non-naturally occurring or engineered composition, vector system, or delivery systems as described herein for treating, preventing, or alleviating an autoimmune disorder. In certain embodiments, the present disclosure relates to the non-naturally occurring or engineered composition, vector system, or delivery systems as described herein for use in treating, preventing, or alleviating an autoimmune disorder. In certain embodiments, the present disclosure relates to a method for treating, preventing, or alleviating an autoimmune disorder comprising introducing or inducing the non-naturally occurring or engineered composition, vector system, or delivery systems as described herein. It is to be understood that preferably the CRISPR-Cas system targets a target specific for the cells responsible for the autoimmune disorder (e.g. specific immune cells).

### In Vitro Apex Labeling

Cellular processes depend on a network of molecular interactions among protein, RNA, and DNA. Accurate detection of protein-DNA and protein-RNA interactions is key to understanding such processes. In vitro proximity labeling technology employs an affinity tag combined with e.g. a photoactivatable probe to label polypeptides and RNAs in the vicinity of a protein or RNA of interest in vitro. After UV irradiation the photoactivatable group reacts with proteins and other molecules that are in close proximity to the tagged molecule, thereby labelling them. Labelled interacting molecules can subsequently be recovered and identified. The Cas protein of the present disclosure can for instance be used to target a probe to a selected RNA sequence.

These applications could also be applied in animal models for in vivo imaging of disease relevant applications or difficult-to culture cell types.

### Synthetic Biology

The development of biological systems has a wide utility, including in clinical applications. It is envisaged that the programmable Cas proteins of the present disclosure can be used fused to split proteins of toxic domains for targeted cell death, for instance using cancer-linked RNA as target transcript. Further, pathways involving protein-protein interaction can be influenced in synthetic biological systems with e.g. fusion complexes with the appropriate effectors such as kinases or other enzymes.

### Protein Splicing: Inteins

Protein splicing is a post-translational process in which an intervening polypeptide, referred to as an intein, catalyzes its own excision from the polypeptides flacking it, referred to as exteins, as well as subsequent ligation of the exteins. The assembly of two or more Cas proteins as described herein on a target transcript could be used to direct the release of a split intein (Topilina and Mills Mob DNA. 2014 Feb. 4; 5(1):5), thereby allowing for direct computation of the existence of a mRNA transcript and subsequent release of a protein product, such as a metabolic enzyme or a transcription factor (for downstream actuation of transcription pathways). This application may have significant relevance in synthetic biology (see above) or large-scale bioproduction (only produce product under certain conditions).

### Inducible, Dosed and Self-Inactivating Systems

In one embodiment, fusion complexes comprising a Cas protein of the present disclosure and an effector component are designed to be inducible, for instance light inducible or chemically inducible. Such inducibility allows for activation of the effector component at a desired moment in time.

Light inducibility is for instance achieved by designing a fusion complex wherein CRY2 PHR/CIBN pairing is used for fusion. This system is particularly useful for light induction of protein interactions in living cells (Konermann S, et al. Nature. 2013; 500:472-476).

Chemical inducibility is for instance provided for by designing a fusion complex wherein FKBP/FRB (FK506 binding protein/FKBP rapamycin binding) pairing is used for fusion. Using this system rapamycin is required for binding of proteins (Zetsche et al. Nat Biotechnol. 2015; 33(2):139-42 describes the use of this system for Cas9).

Further, when introduced in the cell as DNA, the Cas protein of the present disclosures can be modulated by inducible promoters, such as tetracycline or doxycycline controlled transcriptional activation (Tet-On and Tet-Off expression system), hormone inducible gene expression system such as for instance an ecdysone inducible gene expression system and an arabinose-inducible gene expression system. When delivered as RNA, expression of the Cas protein can be modulated via a riboswitch, which can sense a small molecule like tetracycline (as described in Goldfless et al. Nucleic Acids Res. 2012; 40(9):e64).

In one embodiment, the delivery of the Cas protein of the present disclosure can be modulated to change the amount of protein or crRNA in the cell, thereby changing the magnitude of the desired effect or any undesired off-target effects.

In one embodiment, the Cas proteins described herein can be designed to be self-inactivating. When delivered to a cell as RNA, either mRNA or as a replication RNA therapeutic (Wrobleska et al Nat Biotechnol. 2015 August; 33(8): 839-841), they can self-inactivate expression and subsequent effects by destroying the own RNA, thereby reducing residency and potential undesirable effects.

For further in vivo applications of Cas proteins as described herein, reference is made to Mackay J P et al (Nat Struct Mol Biol. 2011 March; 18(3):256-61), Nelles et al (Bioessays. 2015 July; 37(7):732-9) and Abil Z and Zhao H (Mol Biosyst. 2015 October; 11(10):2658-65), which are incorporated herein by reference. In particular, the following applications are envisaged in certain embodiments of the present disclosure, preferably in certain embodiments by using catalytically inactive CRISPR-Cas: enhancing translation (e.g. CRISPR-Cas-translation promotion factor fusions (e.g. eIF4 fusions)); repressing translation (e.g. gRNA targeting ribosome binding sites); exon skipping (e.g. gRNAs targeting splice donor and/or acceptor sites); exon inclusion (e.g. gRNA targeting a particular exon splice donor and/or acceptor site to be included or CRISPR-Cas fused to or recruiting spliceosome components (e.g. U1 snRNA)); accessing RNA localization (e.g. CRISPR-Cas-marker fusions (e.g. EGFP fusions)); altering RNA localization (e.g. CRISPR-Cas-localization signal fusions (e.g. NLS or NES fusions)); RNA degradation (in this case no catalytically inactive CRISPR-Cas is to be used if relied on the activity of CRISPR-Cas, alternatively and for increased specificity, a split CRISPR-Cas may be used); inhibition of non-coding RNA function (e.g. miRNA), such as by degradation or binding of gRNA to functional sites (possibly titrating out at specific sites by relocalization by CRISPR-Cas-signal sequence fusions).

As described herein before and demonstrated in the Examples, CRISPR-Cas function is robust to 5′ or 3′ extensions of the crRNA and to extension of the crRNA loop. It is therefore envisaging that MS2 loops and other recruitment domains can be added to the crRNA without affecting complex formation and binding to target transcripts. Such modifications to the crRNA for recruitment of various effector domains are applicable in the uses of a RNA targeted effector proteins described above.

CRISPR-Cas is capable of mediating resistance to RNA phages. It is therefore envisaged that CRISPR-Cas can be used to immunize, e.g. animals, humans and plants, against RNA-only pathogens, including but not limited to Ebola virus and Zika virus.

In certain embodiments, CRISPR-Cas can process (cleave) its own array. This applies to both the wildtype Cas protein and the mutated Cas protein containing one or more mutated amino acid residues as herein-discussed. It is therefore envisaged that multiple crRNAs designed for different target transcripts and/or applications can be delivered as a single pre-crRNA or as a single transcript driven by one promotor. Such method of delivery has the advantages that it is substantially more compact, easier to synthesize and easier to delivery in viral systems. It will be understood that exact amino acid positions may vary for orthologues of a herein CRISPR-Cas can be adequately determined by protein alignment, as is known in the art, and as described herein elsewhere. Aspects of the present disclosure also encompass methods and uses of the compositions and systems described herein in genome engineering, e.g. for altering or manipulating the expression of one or more genes or the one or more gene products, in prokaryotic or eukaryotic cells, in vitro, in vivo or ex vivo.

In an aspect, the present disclosure provides methods and compositions for modulating, e.g., reducing, expression of a target RNA in cells. In the subject methods, a CRISPR-Cas system of the present disclosure is provided that interferes with transcription, stability, and/or translation of an RNA.

In certain embodiments, an effective amount of CRISPR-Cas system is used to cleave RNA or otherwise inhibit RNA expression. In this regard, the system has uses similar to siRNA and shRNA, thus can also be substituted for such methods. The method includes, without limitation, use of a CRISPR-Cas system as a substitute for e.g., an interfering ribonucleic acid (such as an siRNA or shRNA) or a transcription template thereof, e.g., a DNA encoding an shRNA. The CRISPR-Cas system is introduced into a target cell, e.g., by being administered to a mammal that includes the target cell.

Advantageously, a CRISPR-Cas system of the present disclosure is specific. For example, whereas interfering ribonucleic acid (such as an siRNA or shRNA) polynucleotide systems are plagued by design and stability issues and off-target binding, a CRISPR-Cas system of the present disclosure can be designed with high specificity.

In an aspect of the present disclosure, novel systems also referred to as RNA- or CRISPR systems of the present application are based on herein-identified Cas proteins which do not require the generation of customized proteins to target specific RNA sequences but rather a single enzyme can be programmed by a RNA molecule to recognize a specific RNA target, in other words the enzyme can be recruited to a specific RNA target using said RNA molecule.

In some embodiments, one or more elements of a nucleic acid-targeting system is derived from a particular organism comprising an endogenous CRISPR system. In certain embodiments, the CRISPR system is found in Eubacterium and Ruminococcus. In certain embodiments, the effector protein comprises targeted and collateral ssRNA cleavage activity.

In certain embodiments, the effector protein locus structures include a WYL domain containing accessory protein (so denoted after three amino acids that were conserved in the originally identified group of these domains; see, e.g., WYL domain IPR026881). In certain embodiments, the WYL domain accessory protein comprises at least one helix-turn-helix (HTH) or ribbon-helix-helix (RHH) DNA-binding domain. In certain embodiments, the WYL domain containing accessory protein increases both the targeted and the collateral ssRNA cleavage activity of the Cas protein. In certain embodiments, the WYL domain containing accessory protein comprises an N-terminal RHH domain, as well as a pattern of primarily hydrophobic conserved residues, including an invariant tyrosine-leucine doublet corresponding to the original WYL motif. In certain embodiments, the WYL domain containing accessory protein is WYL1. WYL1 is a single WYL-domain protein associated primarily with Ruminococcus.

### Genome-Wide Knock-Out Screening

The Cas proteins and systems described herein can be used to perform efficient and cost effective functional genomic screens. Such screens can utilize CRISPR-Cas genome wide libraries. Such screens and libraries can provide for determining the function of genes, cellular pathways genes are involved in, and how any alteration in gene expression can result in a particular biological process. An advantage of the present disclosure is that the CRISPR system avoids off-target binding and its resulting side effects. This is achieved using systems arranged to have a high degree of sequence specificity for the target DNA.

A genome wide library may comprise a plurality of CRISPR-Cas system guide RNAs, as described herein, comprising guide sequences that are capable of targeting a plurality of target sequences in a plurality of genomic loci in a population of eukaryotic cells. The population of cells may be a population of embryonic stem (ES) cells. The target sequence in the genomic locus may be a non-coding sequence. The non-coding sequence may be an intron, regulatory sequence, splice site, 3′ UTR, 5′ UTR, or polyadenylation signal. Gene function of one or more gene products may be altered by said targeting. The targeting may result in a knockout of gene function. The targeting of a gene product may comprise more than one guide RNA. A gene product may be targeted by 2, 3, 4, 5, 6, 7, 8, 9, or 10 guide RNAs, preferably 3 to 4 per gene. Off-target modifications may be minimized (See, e.g., DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi:10.1038/nbt.2647 (2013)), incorporated herein by reference. The targeting may be of about 100 or more sequences. The targeting may be of about 1000 or more sequences. The targeting may be of about 20,000 or more sequences. The targeting may be of the entire genome. The targeting may be of a panel of target sequences focused on a relevant or desirable pathway. The pathway may be an immune pathway. The pathway may be a cell division pathway.

One aspect of the present disclosure comprehends a genome wide library that may comprise a plurality of CRISPR-Cas system guide RNAs that may comprise guide sequences that are capable of targeting a plurality of target sequences in a plurality of genomic loci, wherein said targeting results in a knockout of gene function. This library may potentially comprise guide RNAs that target each and every gene in the genome of an organism.

In some embodiments of the present disclosure the organism or subject is a eukaryote (including mammal including human) or a non-human eukaryote or a non-human animal or a non-human mammal. In some embodiments, the organism or subject is a non-human animal, and may be an arthropod, for example, an insect, or may be a nematode. In some methods of the present disclosure the organism or subject is a plant. In some methods of the present disclosure the organism or subject is a mammal or a non-human mammal. A non-human mammal may be for example a rodent (preferably a mouse or a rat), an ungulate, or a primate. In some methods of the present disclosure the organism or subject is algae, including microalgae, or is a fungus.

The knockout of gene function may comprise: introducing into each cell in the population of cells a vector system of one or more vectors comprising an engineered, non-naturally occurring CRISPR-Cas system comprising I. a Cas protein, and II. one or more guide RNAs, wherein components I and II may be same or on different vectors of the system, integrating components I and II into each cell, wherein the guide sequence targets a unique gene in each cell, wherein the Cas protein is operably linked to a regulatory element, wherein when transcribed, the guide RNA comprising the guide sequence directs sequence-specific binding of a CRISPR-Cas system to a target sequence in the genomic loci of the unique gene, inducing cleavage of the genomic loci by the Cas protein, and confirming different knockout mutations in a plurality of unique genes in each cell of the population of cells thereby generating a gene knockout cell library. The present disclosure comprehends that the population of cells is a population of eukaryotic cells, and in a preferred embodiment, the population of cells is a population of embryonic stem (ES) cells.

The one or more vectors may be plasmid vectors. The vector may be a single vector comprising Cas9, a sgRNA, and optionally, a selection marker into target cells. Not being bound by a theory, the ability to simultaneously deliver Cas9 and sgRNA through a single vector enables application to any cell type of interest, without the need to first generate cell lines that express Cas9. The regulatory element may be an inducible promoter. The inducible promoter may be a doxycycline inducible promoter. In some methods of the present disclosure the expression of the guide sequence is under the control of the T7 promoter and is driven by the expression of T7 polymerase. The confirming of different knockout mutations may be by whole exome sequencing. The knockout mutation may be achieved in 100 or more unique genes. The knockout mutation may be achieved in 1000 or more unique genes. The knockout mutation may be achieved in 20,000 or more unique genes. The knockout mutation may be achieved in the entire genome. The knockout of gene function may be achieved in a plurality of unique genes which function in a particular physiological pathway or condition. The pathway or condition may be an immune pathway or condition. The pathway or condition may be a cell division pathway or condition.

The present disclosure also provides kits that comprise the genome wide libraries mentioned herein. The kit may comprise a single container comprising vectors or plasmids comprising the library of the present disclosure. The kit may also comprise a panel comprising a selection of unique CRISPR-Cas system guide RNAs comprising guide sequences from the library of the present disclosure, wherein the selection is indicative of a particular physiological condition. The present disclosure comprehends that the targeting is of about 100 or more sequences, about 1000 or more sequences or about 20,000 or more sequences or the entire genome. Furthermore, a panel of target sequences may be focused on a relevant or desirable pathway, such as an immune pathway or cell division.

### Applications in Plants and Fungi

The compositions, systems, and methods described herein can be used to perform gene or genome interrogation or editing or manipulation in plants and fungi. For example, the applications include investigation and/or selection and/or interrogations and/or comparison and/or manipulations and/or transformation of plant genes or genomes; e.g., to create, identify, develop, optimize, or confer trait(s) or characteristic(s) to plant(s) or to transform a plant or fugus genome. There can accordingly be improved production of plants, new plants with new combinations of traits or characteristics or new plants with enhanced traits. The compositions, systems, and methods can be used with regard to plants in Site-Directed Integration (SDI) or Gene Editing (GE) or any Near Reverse Breeding (NRB) or Reverse Breeding (RB) techniques.

The compositions, systems, and methods herein may be used to confer desired traits (e.g., enhanced nutritional quality, increased resistance to diseases and resistance to biotic and abiotic stress, and increased production of commercially valuable plant products or heterologous compounds) on essentially any plants and fungi, and their cells and tissues. The compositions, systems, and methods may be used to modify endogenous genes or to modify their expression without the permanent introduction into the genome of any foreign gene.

In some embodiments, compositions, systems, and methods may be used in genome editing in plants or where RNAi or similar genome editing techniques have been used previously; see, e.g., Nekrasov, “Plant genome editing made easy: targeted mutagenesis in model and crop plants using the CRISPR-Cas system,” Plant Methods 2013, 9:39 (doi:10.1186/1746-4811-9-39); Brooks, “Efficient gene editing in tomato in the first generation using the CRISPR-Cas9 system,” Plant Physiology September 2014 pp 114.247577; Shan, “Targeted genome modification of crop plants using a CRISPR-Cas system,” Nature Biotechnology 31, 686-688 (2013); Feng, “Efficient genome editing in plants using a CRISPR/Cas system,” Cell Research (2013) 23:1229-1232. doi:10.1038/cr.2013.114; published online 20 Aug. 2013; Xie, “RNA-guided genome editing in plants using a CRISPR-Cas system,” Mol Plant. 2013 November; 6(6):1975-83. doi: 10.1093/mp/sst119. Epub 2013 Aug. 17; Xu, “Gene targeting using the Agrobacterium tumefaciens-mediated CRISPR-Cas system in rice,” Rice 2014, 7:5 (2014), Zhou et al., “Exploiting SNPs for biallelic CRISPR mutations in the outcrossing woody perennial Populus reveals 4-coumarate: CoA ligase specificity and Redundancy,” New Phytologist (2015) (Forum) 1-4 (available online only at www.newphytologist.com); Caliando et al, “Targeted DNA degradation using a CRISPR device stably carried in the host genome, NATURE COMMUNICATIONS 6:6989, DOI: 10.1038/ncomms7989, www.nature.com/naturecommunications DOI: 10.1038/ncomms7989; U.S. Pat. No. 6,603,061—Agrobacterium-Mediated Plant Transformation Method; U.S. Pat. No. 7,868,149—Plant Genome Sequences and Uses Thereof and US 2009/0100536—Transgenic Plants with Enhanced Agronomic Traits, Morrell et al “Crop genomics: advances and applications,” Nat Rev Genet. 2011 Dec. 29; 13(2):85-96, all the contents and disclosure of each of which are herein incorporated by reference in their entirety. Aspects of utilizing the compositions, systems, and methods may be analogous to the use of the composition in plants, and mention is made of the University of Arizona website “CRISPR-PLANT” (www.genome.arizona.edu/crispr/) (supported by Penn State and AGI).

The compositions, systems, and methods may also be used on protoplasts. A “protoplast” refers to a plant cell that has had its protective cell wall completely or partially removed using, for example, mechanical or enzymatic means resulting in an intact biochemical competent unit of living plant that can reform their cell wall, proliferate and regenerate grow into a whole plant under proper growing conditions.

The compositions, systems, and methods may be used for screening genes (e.g., endogenous, mutations) of interest. In some examples, genes of interest include those encoding enzymes involved in the production of a component of added nutritional value or generally genes affecting agronomic traits of interest, across species, phyla, and plant kingdom. By selectively targeting e.g. genes encoding enzymes of metabolic pathways, the genes responsible for certain nutritional aspects of a plant can be identified. Similarly, by selectively targeting genes which may affect a desirable agronomic trait, the relevant genes can be identified. Accordingly, the present disclosure encompasses screening methods for genes encoding enzymes involved in the production of compounds with a particular nutritional value and/or agronomic traits.

It is also understood that reference herein to animal cells may also apply, mutatis mutandis, to plant or fungal cells unless otherwise apparent; and, the enzymes herein having reduced off-target effects and systems employing such enzymes can be used in plant applications, including those mentioned herein.

In some cases, nucleic acids introduced to plants and fungi may be codon optimized for expression in the plants and fungi. Methods of codon optimization include those described in Kwon K C, et al., Codon Optimization to Enhance Expression Yields Insights into Chloroplast Translation, Plant Physiol. 2016 September; 172(1):62-77.

The components (e.g., nucleic acid-guided nuclease) in the compositions and systems may further comprise one or more functional domains described herein. In some examples, the functional domains may be an exonuclease. Such exonuclease may increase the efficiency of the nucleic acid-guided nuclease' function, e.g., mutagenesis efficiency. An example of the functional domain is Trex2, as described in Weiss T et al., www.biorxiv.org/content/10.1101/2020.04.11.037572v1, doi: doi.org/10.1101/2020.04.11.037572.

### Examples of Plants

The compositions, systems, and methods herein can be used to confer desired traits on essentially any plant. A wide variety of plants and plant cell systems may be engineered for the desired physiological and agronomic characteristics. In general, the term “plant” relates to any various photosynthetic, eukaryotic, unicellular or multicellular organism of the kingdom Plantae characteristically growing by cell division, containing chloroplasts, and having cell walls comprised of cellulose. The term plant encompasses monocotyledonous and dicotyledonous plants.

The compositions, systems, and methods may be used over a broad range of plants, such as for example with dicotyledonous plants belonging to the orders Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales; monocotyledonous plants such as those belonging to the orders Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales, or with plants belonging to Gymnospermae, e.g., those belonging to the orders Pinales, Ginkgoales, Cycadales, Araucariales, Cupressales and Gnetales.

The compositions, systems, and methods herein can be used over a broad range of plant species, included in the non-limitative list of dicot, monocot or gymnosperm genera hereunder: Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Trifolium, Trigonella, Vicia, Vinca, Vilis, and Vigna; and the genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, Zea, Abies, Cunninghamia, Ephedra, Picea, Pinus, and Pseudotsuga.

In some embodiments, target plants and plant cells for engineering include those monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). Specifically, the plants are intended to comprise without limitation angiosperm and gymnosperm plants such as acacia, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, and zucchini.

The term plant also encompasses Algae, which are mainly photoautotrophs unified primarily by their lack of roots, leaves and other organs that characterize higher plants. The compositions, systems, and methods can be used over a broad range of “algae” or “algae cells.” Examples of algae include eukaryotic phyla, including the Rhodophyta (red algae), Chlorophyta (green algae), Phaeophyta (brown algae), Bacillariophyta (diatoms), Eustigmatophyta and dinoflagellates as well as the prokaryotic phylum Cyanobacteria (blue-green algae). Examples of algae species include those of Amphora, Anabaena, Anikstrodesmis, Botryococcus, Chaetoceros, Chlamydomonas, Chlorella, Chlorococcum, Cyclotella, Cylindrotheca, Dunaliella, Emiliana, Euglena, Hematococcus, Isochrysis, Monochrysis, Monoraphidium, Nannochloris, Nannnochloropsis, Navicula, Nephrochloris, Nephroselmis, Nitzschia, Nodularia, Nostoc, Oochromonas, Oocystis, Oscillartoria, Pavlova, Phaeodactylum, Playtmonas, Pleurochrysis, Porhyra, Pseudoanabaena, Pyramimonas, Stichococcus, Synechococcus, Synechocystis, Tetraselmis, Thalassiosira, and Trichodesmium.

### Plant Promoters

In order to ensure appropriate expression in a plant cell, the components of the components and systems herein may be placed under control of a plant promoter. A plant promoter is a promoter operable in plant cells. A plant promoter is capable of initiating transcription in plant cells, whether or not its origin is a plant cell. The use of different types of promoters is envisaged.

In some examples, the plant promoter is a constitutive plant promoter, which is a promoter that is able to express the open reading frame (ORF) that it controls in all or nearly all of the plant tissues during all or nearly all developmental stages of the plant (referred to as “constitutive expression”). One example of a constitutive promoter is the cauliflower mosaic virus 35S promoter. In some examples, the plant promoter is a regulated promoter, which directs gene expression not constitutively, but in a temporally- and/or spatially-regulated manner, and includes tissue-specific, tissue-preferred and inducible promoters. Different promoters may direct the expression of a gene in different tissues or cell types, or at different stages of development, or in response to different environmental conditions. In some examples, the plant promoter is a tissue-preferred promoters, which can be utilized to target enhanced expression in certain cell types within a particular plant tissue, for instance vascular cells in leaves or roots or in specific cells of the seed.

Exemplary plant promoters include those obtained from plants, plant viruses, and bacteria such as Agrobacterium or Rhizobium which comprise genes expressed in plant cells. Additional examples of promoters include those described in Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-91.

In some examples, a plant promoter may be an inducible promoter, which is inducible and allows for spatiotemporal control of gene editing or gene expression may use a form of energy. The form of energy may include sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome), such as a Light Inducible Transcriptional Effector (LITE) that direct changes in transcriptional activity in a sequence-specific manner. In a particular example, of the components of a light inducible system include a nucleic acid-guided nuclease, a light-responsive cytochrome heterodimer (e.g. from Arabidopsis thaliana), and a transcriptional activation/repression domain.

In some examples, the promoter may be a chemical-regulated promotor (where the application of an exogenous chemical induces gene expression) or a chemical-repressible promoter (where application of the chemical represses gene expression). Examples of chemical-inducible promoters include maize ln2-2 promoter (activated by benzene sulfonamide herbicide safeners), the maize GST promoter (activated by hydrophobic electrophilic compounds used as pre-emergent herbicides), the tobacco PR-1 a promoter (activated by salicylic acid), promoters regulated by antibiotics (such as tetracycline-inducible and tetracycline-repressible promoters).

### Stable Integration in the Genome of Plants

In some embodiments, polynucleotides encoding the components of the compositions and systems may be introduced for stable integration into the genome of a plant cell. In some cases, vectors or expression systems may be used for such integration. The design of the vector or the expression system can be adjusted depending on for when, where and under what conditions the guide RNA and/or the nucleic acid-guided nuclease gene are expressed. In some cases, the polynucleotides may be integrated into an organelle of a plant, such as a plastid, mitochondrion or a chloroplast. The elements of the expression system may be on one or more expression constructs which are either circular such as a plasmid or transformation vector, or non-circular such as linear double stranded DNA.

In some embodiments, the method of integration generally comprises the steps of selecting a suitable host cell or host tissue, introducing the construct(s) into the host cell or host tissue, and regenerating plant cells or plants therefrom. In some examples, the expression system for stable integration into the genome of a plant cell may contain one or more of the following elements: a promoter element that can be used to express the RNA and/or nucleic acid-guided nuclease in a plant cell; a 5′ untranslated region to enhance expression; an intron element to further enhance expression in certain cells, such as monocot cells; a multiple-cloning site to provide convenient restriction sites for inserting the guide RNA and/or the nucleic acid-guided nuclease gene sequences and other desired elements; and a 3′ untranslated region to provide for efficient termination of the expressed transcript.

### Transient Expression in Plants

In some embodiments, the components of the compositions and systems may be transiently expressed in the plant cell. In some examples, the compositions and systems may modify a target nucleic acid only when both the guide RNA and the nucleic acid-guided nuclease are present in a cell, such that genomic modification can further be controlled. As the expression of the nucleic acid-guided nuclease is transient, plants regenerated from such plant cells typically contain no foreign DNA. In certain examples, the nucleic acid-guided nuclease is stably expressed and the guide sequence is transiently expressed.

DNA and/or RNA (e.g., mRNA) may be introduced to plant cells for transient expression. In such cases, the introduced nucleic acid may be provided in sufficient quantity to modify the cell but do not persist after a contemplated period of time has passed or after one or more cell divisions.

The transient expression may be achieved using suitable vectors. Exemplary vectors that may be used for transient expression include a pEAQ vector (may be tailored for Agrobacterium-mediated transient expression) and Cabbage Leaf Curl virus (CaLCuV), and vectors described in Sainsbury F. et al., Plant Biotechnol J. 2009 September; 7(7):682-93; and Yin K et al., Scientific Reports volume 5, Article number: 14926 (2015).

Combinations of the different methods described above are also envisaged.

Translocation to and/or Expression in Specific Plant Organelles

The compositions and systems herein may comprise elements for translocation to and/or expression in a specific plant organelle.

### Chloroplast Targeting

In some embodiments, it is envisaged that the compositions and systems are used to specifically modify chloroplast genes or to ensure expression in the chloroplast. The compositions and systems (e.g., nucleic acid-guided nuclease, guide molecules, or their encoding polynucleotides) may be transformed, compartmentalized, and/or targeted to the chloroplast. In an example, the introduction of genetic modifications in the plastid genome can reduce biosafety issues such as gene flow through pollen.

Examples of methods of chloroplast transformation include Particle bombardment, PEG treatment, and microinjection, and the translocation of transformation cassettes from the nuclear genome to the plastid. In some examples, targeting of chloroplasts may be achieved by incorporating in chloroplast localization sequence, and/or the expression construct a sequence encoding a chloroplast transit peptide (CTP) or plastid transit peptide, operably linked to the 5′ region of the sequence encoding the components of the compositions and systems. Additional examples of transforming, targeting and localization of chloroplasts include those described in WO2010061186, Protein Transport into Chloroplasts, 2010, Annual Review of Plant Biology, Vol. 61: 157-180, and US 20040142476, which are incorporated by reference herein in their entireties.

### Exemplary Applications in Plants

The compositions, systems, and methods may be used to generate genetic variation(s) in a plant (e.g., crop) of interest. One or more, e.g., a library of, guide molecules targeting one or more locations in a genome may be provided and introduced into plant cells together with the nucleic acid-guided nuclease. For example, a collection of genome-scale point mutations and gene knock-outs can be generated. In some examples, the compositions, systems, and methods may be used to generate a plant part or plant from the cells so obtained and screening the cells for a trait of interest. The target genes may include both coding and non-coding regions. In some cases, the trait is stress tolerance and the method is a method for the generation of stress-tolerant crop varieties.

In some embodiments, the compositions, systems, and methods are used to modify endogenous genes or to modify their expression. The expression of the components may induce targeted modification of the genome, either by direct activity of the nucleic acid-guided nuclease and optionally introduction of recombination template DNA, or by modification of genes targeted. The different strategies described herein above allow nucleic acid-guided nuclease-mediated targeted genome editing without requiring the introduction of the components into the plant genome.

In some cases, the modification may be performed without the permanent introduction into the genome of the plant of any foreign gene, including those encoding components of the composition herein, so as to avoid the presence of foreign DNA in the genome of the plant. This can be of interest as the regulatory requirements for non-transgenic plants are less rigorous. Components which are transiently introduced into the plant cell are typically removed upon crossing.

For example, the modification may be performed by transient expression of the components of the compositions and systems. The transient expression may be performed by delivering the components of the compositions and systems with viral vectors, delivery into protoplasts, with the aid of particulate molecules such as nanoparticles or CPPs.

Generation of Plants with Desired Traits

The compositions, systems, and methods herein may be used to introduce desired traits to plants. The approaches include introduction of one or more foreign genes to confer a trait of interest, editing or modulating endogenous genes to confer a trait of interest.

### Agronomic Traits

In some embodiments, crop plants can be improved by influencing specific plant traits. Examples of the traits include improved agronomic traits such as herbicide resistance, disease resistance, abiotic stress tolerance, high yield, and superior quality, pesticide-resistance, disease resistance, insect and nematode resistance, resistance against parasitic weeds, drought tolerance, nutritional value, stress tolerance, self-pollination voidance, forage digestibility biomass, and grain yield.

In some embodiments, genes that confer resistance to pests or diseases may be introduced to plants. In cases there are endogenous genes that confer such resistance in a plants, their expression and function may be enhanced (e.g., by introducing extra copies, modifications that enhance expression and/or activity).

Examples of genes that confer resistance include plant disease resistance genes (e.g., Cf-9, Pto, RSP2, SlDMR6-1), genes conferring resistance to a pest (e.g., those described in WO96/30517), Bacillus thuringiensis proteins, lectins, Vitamin-binding proteins (e.g., avidin), enzyme inhibitors (e.g., protease or proteinase inhibitors or amylase inhibitors), insect-specific hormones or pheromones (e.g., ecdysteroid or a juvenile hormone, variant thereof, a mimetic based thereon, or an antagonist or agonist thereof) or genes involved in the production and regulation of such hormone and pheromones, insect-specific peptides or neuropeptide, Insect-specific venom (e.g., produced by a snake, a wasp, etc., or analog thereof), Enzymes responsible for a hyperaccumulation of a monoterpene, a sesquiterpene, a steroid, hydroxamic acid, a phenylpropanoid derivative or another nonprotein molecule with insecticidal activity, Enzymes involved in the modification of biologically active molecule (e.g., a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic), molecules that stimulates signal transduction, Viral-invasive proteins or a complex toxin derived therefrom, Developmental-arrestive proteins produced in nature by a pathogen or a parasite, a developmental-arrestive protein produced in nature by a plant, or any combination thereof.

The compositions, systems, and methods may be used to identify, screen, introduce or remove mutations or sequences lead to genetic variability that give rise to susceptibility to certain pathogens, e.g., host specific pathogens. Such approach may generate plants that are non-host resistance, e.g., the host and pathogen are incompatible or there can be partial resistance against all races of a pathogen, typically controlled by many genes and/or also complete resistance to some races of a pathogen but not to other races.

In some embodiments, the compositions, systems, and methods may be used to modify genes involved in plant diseases. Such genes may be removed, inactivated, or otherwise regulated or modified. Examples of plant diseases include those described in [0045]-[0080] of US20140213619A1, which is incorporated by reference herein in its entirety.

In some embodiments, genes that confer resistance to herbicides may be introduced to plants. Examples of genes that confer resistance to herbicides include genes conferring resistance to herbicides that inhibit the growing point or meristem, such as an imidazolinone or a sulfonylurea, genes conferring glyphosate tolerance (e.g., resistance conferred by, e.g., mutant 5-enolpyruvylshikimate-3-phosphate synthase genes, aroA genes and glyphosate acetyl transferase (GAT) genes, respectively), or resistance to other phosphono compounds such as by glufosinate (phosphinothricin acetyl transferase (PAT) genes from Streptomyces species, including Streptomyces hygroscopicus and Streptomyces viridichromogenes), and to pyridinoxy or phenoxy proprionic acids and cyclohexones by ACCase inhibitor-encoding genes), genes conferring resistance to herbicides that inhibit photosynthesis (such as a triazine (psbA and gs+ genes) or a benzonitrile (nitrilase gene), and glutathione S-transferase), genes encoding enzymes detoxifying the herbicide or a mutant glutamine synthase enzyme that is resistant to inhibition, genes encoding a detoxifying enzyme is an enzyme encoding a phosphinothricin acetyltransferase (such as the bar or pat protein from Streptomyces species), genes encoding hydroxyphenylpyruvatedioxygenases (HPPD) inhibitors, e.g., naturally occurring HPPD resistant enzymes, and genes encoding a mutated or chimeric HPPD enzyme.

In some embodiments, genes involved in Abiotic stress tolerance may be introduced to plants. Examples of genes include those capable of reducing the expression and/or the activity of poly(ADP-ribose) polymerase (PARP) gene, transgenes capable of reducing the expression and/or the activity of the PARG encoding genes, genes coding for a plant-functional enzyme of the nicotineamide adenine dinucleotide salvage synthesis pathway including nicotinamidase, nicotinate phosphoribosyltransferase, nicotinic acid mononucleotide adenyl transferase, nicotinamide adenine dinucleotide synthetase or nicotine amide phosphorybosyltransferase, enzymes involved in carbohydrate biosynthesis, enzymes involved in the production of polyfructose (e.g., the inulin and levan-type), the production of alpha-1,6 branched alpha-1,4-glucans, the production of alternan, the production of hyaluronan.

In some embodiments, genes that improve drought resistance may be introduced to plants. Examples of genes Ubiquitin Protein Ligase protein (UPL) protein (UPL3), DR02, DR03, ABC transporter, and DREB1A.

### Nutritionally Improved Plants

In some embodiments, the compositions, systems, and methods may be used to produce nutritionally improved plants. In some examples, such plants may provide functional foods, e.g., a modified food or food ingredient that may provide a health benefit beyond the traditional nutrients it contains. In certain examples, such plants may provide nutraceuticals foods, e.g., substances that may be considered a food or part of a food and provides health benefits, including the prevention and treatment of disease. The nutraceutical foods may be useful in the prevention and/or treatment of diseases in animals and humans, e.g., cancers, diabetes, cardiovascular disease, and hypertension.

An improved plant may naturally produce one or more desired compounds and the modification may enhance the level or activity or quality of the compounds. In some cases, the improved plant may not naturally produce the compound(s), while the modification enables the plant to produce such compound(s). In some cases, the compositions, systems, and methods used to modify the endogenous synthesis of these compounds indirectly, e.g. by modifying one or more transcription factors that controls the metabolism of this compound.

Examples of nutritionally improved plants include plants comprising modified protein quality, content and/or amino acid composition, essential amino acid contents, oils and fatty acids, carbohydrates, vitamins and carotenoids, functional secondary metabolites, and minerals. In some examples, the improved plants may comprise or produce compounds with health benefits. Examples of nutritionally improved plants include those described in Newell-McGloughlin, Plant Physiology, July 2008, Vol. 147, pp. 939-953.

Examples of compounds that can be produced include carotenoids (e.g., α-Carotene or β-Carotene), lutein, lycopene, Zeaxanthin, Dietary fiber (e.g., insoluble fibers, β-Glucan, soluble fibers, fatty acids (e.g., ω-3 fatty acids, Conjugated linoleic acid, GLA,), Flavonoids (e.g., Hydroxycinnamates, flavonols, catechins and tannins), Glucosinolates, indoles, isothiocyanates (e.g., Sulforaphane), Phenolics (e.g., stilbenes, caffeic acid and ferulic acid, epicatechin), Plant stanols/sterols, Fructans, inulins, fructo-oligosaccharides, Saponins, Soybean proteins, Phytoestrogens (e.g., isoflavones, lignans), Sulfides and thiols such as diallyl sulphide, Allyl methyl trisulfide, dithiolthiones, Tannins, such as proanthocyanidins, or any combination thereof.

The compositions, systems, and methods may also be used to modify protein/starch functionality, shelf life, taste/aesthetics, fiber quality, and allergen, antinutrient, and toxin reduction traits.

Examples of genes and nucleic acids that can be modified to introduce the traits include stearyl-ACP desaturase, DNA associated with the single allele which may be responsible for maize mutants characterized by low levels of phytic acid, Tf RAP2.2 and its interacting partner SINAT2, Tf Dof1, and DOF Tf AtDof1.1 (OBP2).

### Modification of Polyploid Plants

The compositions, systems, and methods may be used to modify polyploid plants. Polyploid plants carry duplicate copies of their genomes (e.g. as many as six, such as in wheat). In some cases, the compositions, systems, and methods may be can be multiplexed to affect all copies of a gene, or to target dozens of genes at once. For instance, the compositions, systems, and methods may be used to simultaneously ensure a loss of function mutation in different genes responsible for suppressing defenses against a disease. The modification may be simultaneous suppression the expression of the TaMLO-A1, TaMLO-B1 and TaMLO-D1 nucleic acid sequence in a wheat plant cell and regenerating a wheat plant therefrom, in order to ensure that the wheat plant is resistant to powdery mildew (e.g., as described in WO2015109752).

### Regulation of Fruit-Ripening

The compositions, systems, and methods may be used to regulate ripening of fruits. Ripening is a normal phase in the maturation process of fruits and vegetables. Only a few days after it starts it may render a fruit or vegetable inedible, which can bring significant losses to both farmers and consumers.

In some embodiments, the compositions, systems, and methods are used to reduce ethylene production. In some examples, the compositions, systems, and methods may be used to suppress the expression and/or activity of ACC synthase, insert a ACC deaminase gene or a functional fragment thereof, insert a SAM hydrolase gene or functional fragment thereof, suppress ACC oxidase gene expression

Alternatively or additionally, the compositions, systems, and methods may be used to modify ethylene receptors (e.g., suppressing ETR1) and/or Polygalacturonase (PG). Suppression of a gene may be achieved by introducing a mutation, an antisense sequence, and/or a truncated copy of the gene to the genome.

### Increasing Storage Life of Plants

In some embodiments, the compositions, systems, and methods are used to modify genes involved in the production of compounds which affect storage life of the plant or plant part. The modification may be in a gene that prevents the accumulation of reducing sugars in potato tubers. Upon high-temperature processing, these reducing sugars react with free amino acids, resulting in brown, bitter-tasting products and elevated levels of acrylamide, which is a potential carcinogen. In particular embodiments, the methods provided herein are used to reduce or inhibit expression of the vacuolar invertase gene (VInv), which encodes a protein that breaks down sucrose to glucose and fructose.

### Reducing Allergens in Plants

In some embodiments, the compositions, systems, and methods are used to generate plants with a reduced level of allergens, making them safer for consumers. To this end, the compositions, systems, and methods may be used to identify and modify (e.g., suppress) one or more genes responsible for the production of plant allergens. Examples of such genes include Lol p5, as well as those in peanuts, soybeans, lentils, peas, lupin, green beans, mung beans, such as those described in Nicolaou et al., Current Opinion in Allergy and Clinical Immunology 2011; 11(3):222), which is incorporated by reference herein in its entirety.

### Generation of Male Sterile Plants

The compositions, systems, and methods may be used to generate male sterile plants. Hybrid plants typically have advantageous agronomic traits compared to inbred plants. However, for self-pollinating plants, the generation of hybrids can be challenging. In different plant types (e.g., maize and rice), genes have been identified which are important for plant fertility, more particularly male fertility. Plants that are as such genetically altered can be used in hybrid breeding programs.

The compositions, systems, and methods may be used to modify genes involved male fertility, e.g., inactivating (such as by introducing mutations to) genes required for male fertility. Examples of the genes involved in male fertility include cytochrome P450-like gene (MS26) or the meganuclease gene (MS45), and those described in Wan X et al., Mol Plant. 2019 Mar. 4; 12(3):321-342; and Kim Y J, et al., Trends Plant Sci. 2018 January; 23(1):53-65.

### Increasing the Fertility Stage in Plants

In some embodiments, the compositions, systems, and methods may be used to prolong the fertility stage of a plant such as of a rice. For instance, a rice fertility stage gene such as Ehd3 can be targeted in order to generate a mutation in the gene and plantlets can be selected for a prolonged regeneration plant fertility stage.

### Production of Early Yield of Products

In some embodiments, the compositions, systems, and methods may be used to produce early yield of the product. For example, flowering process may be modulated, e.g., by mutating flowering repressor gene such as SP5G. Examples of such approaches include those described in Soyk S, et al., Nat Genet. 2017 January; 49(1):162-168.

### Oil and Biofuel Production

The compositions, systems, and methods may be used to generate plants for oil and biofuel production. Biofuels include fuels made from plant and plant-derived resources. Biofuels may be extracted from organic matter whose energy has been obtained through a process of carbon fixation or are made through the use or conversion of biomass. This biomass can be used directly for biofuels or can be converted to convenient energy containing substances by thermal conversion, chemical conversion, and biochemical conversion. This biomass conversion can result in fuel in solid, liquid, or gas form. Biofuels include bioethanol and biodiesel. Bioethanol can be produced by the sugar fermentation process of cellulose (starch), which may be derived from maize and sugar cane. Biodiesel can be produced from oil crops such as rapeseed, palm, and soybean. Biofuels can be used for transportation.

### Generation of Plants for Production of Vegetable Oils and Biofuels

The compositions, systems, and methods may be used to generate algae (e.g., diatom) and other plants (e.g., grapes) that express or overexpress high levels of oil or biofuels.

In some cases, the compositions, systems, and methods may be used to modify genes involved in the modification of the quantity of lipids and/or the quality of the lipids. Examples of such genes include those involved in the pathways of fatty acid synthesis, e.g., acetyl-CoA carboxylase, fatty acid synthase, 3-ketoacyl_acyl-carrier protein synthase III, glycerol-3-phospate deshydrogenase (G3PDH), Enoyl-acyl carrier protein reductase (Enoyl-ACP-reductase), glycerol-3-phosphate acyltransferase, lysophosphatidic acyl transferase or diacylglycerol acyltransferase, phospholipid: diacylglycerol acyltransferase, phoshatidate phosphatase, fatty acid thioesterase such as palmitoyi protein thioesterase, or malic enzyme activities.

In further embodiments, it is envisaged to generate diatoms that have increased lipid accumulation. This can be achieved by targeting genes that decrease lipid catabolization. Examples of genes include those involved in the activation of triacylglycerol and free fatty acids, β-oxidation of fatty acids, such as genes of acyl-CoA synthetase, 3-ketoacyl-CoA thiolase, acyl-CoA oxidase activity and phosphoglucomutase.

In some examples, algae may be modified for production of oil and biofuels, including fatty acids (e.g., fatty esters such as acid methyl esters (FAME) and fatty acid ethyl esters (FAEE)). Examples of methods of modifying microalgae include those described in Stovicek et al. Metab. Eng. Comm., 2015; 2:1; U.S. Pat. No. 8,945,839; and International Patent Publication No. WO 2015/086795.

In some examples, one or more genes may be introduced (e.g., overexpressed) to the plants (e.g., algae) to produce oils and biofuels (e.g., fatty acids) from a carbon source (e.g., alcohol). Examples of the genes include genes encoding acyl-CoA synthases, ester synthases, thioesterases (e.g., tesA, ‘tesA, tesB, fatB, fatB2, fatB3, fatA1, or fatA), acyl-CoA synthases (e.g., fadD, JadK, BH3103, pfl-4354, EAV15023, fadD1, fadD2, RPC_4074, fadDD35, fadDD22, faa39), ester synthases (e.g., synthase/acyl-CoA:diacylglycerl acyltransferase from Simmondsia chinensis, Acinetobacter sp. ADP, Alcanivorax borkumensis, Pseudomonas aeruginosa, Fundibacter jadensis, Arabidopsis thaliana, or Alkaligenes eutrophus, or variants thereof).

Additionally or alternatively, one or more genes in the plants (e.g., algae) may be inactivated (e.g., expression of the genes is decreased). For examples, one or more mutations may be introduced to the genes. Examples of such genes include genes encoding acyl-CoA dehydrogenases (e.g., fade), outer membrane protein receptors, and transcriptional regulator (e.g., repressor) of fatty acid biosynthesis (e.g., fabR), pyruvate formate lyases (e.g., pflB), lactate dehydrogenases (e.g., IdhA).

### Organic Acid Production

In some embodiments, plants may be modified to produce organic acids such as lactic acid. The plants may produce organic acids using sugars, pentose or hexose sugars. To this end, one or more genes may be introduced (e.g., and overexpressed) in the plants. An example of such genes include LDH gene.

In some examples, one or more genes may be inactivated (e.g., expression of the genes is decreased). For examples, one or more mutations may be introduced to the genes. The genes may include those encoding proteins involved an endogenous metabolic pathway which produces a metabolite other than the organic acid of interest and/or wherein the endogenous metabolic pathway consumes the organic acid.

Examples of genes that can be modified or introduced include those encoding pyruvate decarboxylases (pdc), fumarate reductases, alcohol dehydrogenases (adh), acetaldehyde dehydrogenases, phosphoenolpyruvate carboxylases (ppc), D-lactate dehydrogenases (d-ldh), L-lactate dehydrogenases (l-ldh), lactate 2-monooxygenases, lactate dehydrogenase, cytochrome-dependent lactate dehydrogenases (e.g., cytochrome B2-dependent L-lactate dehydrogenases).

### Enhancing Plant Properties for Biofuel Production

In some embodiments, the compositions, systems, and methods are used to alter the properties of the cell wall of plants to facilitate access by key hydrolyzing agents for a more efficient release of sugars for fermentation. By reducing the proportion of lignin in a plant the proportion of cellulose can be increased. In particular embodiments, lignin biosynthesis may be downregulated in the plant so as to increase fermentable carbohydrates.

In some examples, one or more lignin biosynthesis genes may be down regulated. Examples of such genes include 4-coumarate 3-hydroxylases (C3H), phenylalanine ammonia-lyases (PAL), cinnamate 4-hydroxylases (C4H), hydroxycinnamoyl transferases (HCT), caffeic acid O-methyltransferases (COMT), caffeoyl CoA 3-O-methyltransferases (CCoAOMT), ferulate 5-hydroxylases (F5H), cinnamyl alcohol dehydrogenases (CAD), cinnamoyl CoA-reductases (CCR), 4-coumarate-CoA ligases (4CL), monolignol-lignin-specific glycosyltransferases, and aldehyde dehydrogenases (ALDH), and those described in WO 2008064289.

In some examples, plant mass that produces lower level of acetic acid during fermentation may be reduced. To this end, genes involved in polysaccharide acetylation (e.g., Cas1L and those described in WO 2010096488) may be inactivated.

### Other Microorganisms for Oils and Biofuel Production

In some embodiments, microorganisms other than plants may be used for production of oils and biofuels using the compositions, systems, and methods herein. Examples of the microorganisms include those of the genus of Escherichia, Bacillus, Lactobacillus, Rhodococcus, Synechococcus, Synechoystis, Pseudomonas, Aspergillus, Trichoderma, Neurospora, Fusarium, Humicola, Rhizomucor, Kluyveromyces, Pichia, Mucor, Myceliophtora, Penicillium, Phanerochaete, Pleurotus, Trametes, Chrysosporium, Saccharomyces, Stenotrophamonas, Schizosaccharomyces, Yarrowia, or Streptomyces.

### Plant Cultures and Regeneration

In some embodiments, the modified plants or plant cells may be cultured to regenerate a whole plant which possesses the transformed or modified genotype and thus the desired phenotype. Examples of regeneration techniques include those relying on manipulation of certain phytohormones in a tissue culture growth medium, relying on a biocide and/or herbicide marker which has been introduced together with the desired nucleotide sequences, obtaining from cultured protoplasts, plant callus, explants, organs, pollens, embryos or parts thereof.

### Detecting Modifications in the Plant Genome-Selectable Markers

When the compositions, systems, and methods are used to modify a plant, suitable methods may be used to confirm and detect the modification made in the plant. In some examples, when a variety of modifications are made, one or more desired modifications or traits resulting from the modifications may be selected and detected. The detection and confirmation may be performed by biochemical and molecular biology techniques such as Southern analysis, PCR, Northern blot, S1 RNase protection, primer-extension or reverse transcriptase-PCR, enzymatic assays, ribozyme activity, gel electrophoresis, Western blot, immunoprecipitation, enzyme-linked immunoassays, in situ hybridization, enzyme staining, and immunostaining.

In some cases, one or more markers, such as selectable and detectable markers, may be introduced to the plants. Such markers may be used for selecting, monitoring, isolating cells and plants with desired modifications and traits. A selectable marker can confer positive or negative selection and is conditional or non-conditional on the presence of external substrates. Examples of such markers include genes and proteins that confer resistance to antibiotics, such as hygromycin (hpt) and kanamycin (nptII), and genes that confer resistance to herbicides, such as phosphinothricin (bar) and chlorosulfuron (als), enzyme capable of producing or processing a colored substances (e.g., the β-glucuronidase, luciferase, B or C1 genes).

### Applications in Fungi

The compositions, systems, and methods described herein can be used to perform efficient and cost effective gene or genome interrogation or editing or manipulation in fungi or fungal cells, such as yeast. The approaches and applications in plants may be applied to fungi as well.

A fungal cell may be any type of eukaryotic cell within the kingdom of fungi, such as phyla of Ascomycota, Basidiomycota, Blastocladiomycota, Chytridiomycota, Glomeromycota, Microsporidia, and Neocallimastigomycota. Examples of fungi or fungal cells in include yeasts, molds, and filamentous fungi.

In some embodiments, the fungal cell is a yeast cell. A yeast cell refers to any fungal cell within the phyla Ascomycota and Basidiomycota. Examples of yeasts include budding yeast, fission yeast, and mold, S. cerervisiae, Kluyveromyces marxianus, Issatchenkia orientalis, Candida spp. (e.g., Candida albicans), Yarrowia spp. (e.g., Yarrowia hpolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientalis, Pichia kudriavzevii and Candida acidothermophilum).

In some embodiments, the fungal cell is a filamentous fungal cell, which grow in filaments, e.g., hyphae or mycelia. Examples of filamentous fungal cells include Aspergillus spp. (e.g., Aspergillus niger), Trichoderma spp. (e.g., Trichoderma reesei), Rhizopus spp. (e.g., Rhizopus oryzae), and Mortierella spp. (e.g., Mortierella isabellina).

In some embodiments, the fungal cell is of an industrial strain. Industrial strains include any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes include fermentation (e.g., in production of food or beverage products), distillation, biofuel production, production of a compound, and production of a polypeptide. Examples of industrial strains include, without limitation, JAY270 and ATCC4124.

In some embodiments, the fungal cell is a polyploid cell whose genome is present in more than one copy. Polyploid cells include cells naturally found in a polyploid state, and cells that has been induced to exist in a polyploid state (e.g., through specific regulation, alteration, inactivation, activation, or modification of meiosis, cytokinesis, or DNA replication). A polyploid cell may be a cell whose entire genome is polyploid, or a cell that is polyploid in a particular genomic locus of interest. In some examples, the abundance of guide RNA may more often be a rate-limiting component in genome engineering of polyploid cells than in haploid cells, and thus the methods using the composition described herein may take advantage of using certain fungal cell types.

In some embodiments, the fungal cell is a diploid cell, whose genome is present in two copies. Diploid cells include cells naturally found in a diploid state, and cells that have been induced to exist in a diploid state (e.g., through specific regulation, alteration, inactivation, activation, or modification of meiosis, cytokinesis, or DNA replication). A diploid cell may refer to a cell whose entire genome is diploid, or it may refer to a cell that is diploid in a particular genomic locus of interest.

In some embodiments, the fungal cell is a haploid cell, whose genome is present in one copy. Haploid cells include cells naturally found in a haploid state, or cells that have been induced to exist in a haploid state (e.g., through specific regulation, alteration, inactivation, activation, or modification of meiosis, cytokinesis, or DNA replication). A haploid cell may refer to a cell whose entire genome is haploid, or it may refer to a cell that is haploid in a particular genomic locus of interest.

The compositions and systems, and nucleic acid encoding thereof may be introduced to fungi cells using the delivery systems and methods herein. Examples of delivery systems include lithium acetate treatment, bombardment, electroporation, and those described in Kawai et al., 2010, Bioeng Bugs. 2010 November-December; 1(6): 395-403.

In some examples, a yeast expression vector (e.g., those with one or more regulatory elements) may be used. Examples of such vectors include a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2μ plasmids, yeast integrative plasmids, yeast replicative plasmids, shuttle vectors, and episomal plasmids.

### Biofuel and Materials Production by Fungi

In some embodiments, the compositions, systems, and methods may be used for generating modified fungi for biofuel and material productions. For instance, the modified fungi for production of biofuel or biopolymers from fermentable sugars and optionally to be able to degrade plant-derived lignocellulose derived from agricultural waste as a source of fermentable sugars. Foreign genes required for biofuel production and synthesis may be introduced in to fungi In some examples, the genes may encode enzymes involved in the conversion of pyruvate to ethanol or another product of interest, degrade cellulose (e.g., cellulase), endogenous metabolic pathways which compete with the biofuel production pathway.

In some examples, the compositions, systems, and methods may be used for generating and/or selecting yeast strains with improved xylose or cellobiose utilization, isoprenoid biosynthesis, and/or lactic acid production. One or more genes involved in the metabolism and synthesis of these compounds may be modified and/or introduced to yeast cells. Examples of the methods and genes include lactate dehydrogenase, PDC1 and PDC5, and those described in Ha, S. J., et al. (2011) Proc. Natl. Acad. Sci. USA 108(2):504-9 and Galazka, J. M., et al. (2010) Science 330(6000):84-6; Jakočiūnas T et al., Metab Eng. 2015 March; 28:213-222; Stovicek V, et al., FEMS Yeast Res. 2017 Aug. 1; 17(5).

### Improved Plants and Yeast Cells

The present disclosure further provides improved plants and fungi. The improved and fungi may comprise one or more genes introduced, and/or one or more genes modified by the compositions, systems, and methods herein. The improved plants and fungi may have increased food or feed production (e.g., higher protein, carbohydrate, nutrient or vitamin levels), oil and biofuel production (e.g., methanol, ethanol), tolerance to pests, herbicides, drought, low or high temperatures, excessive water, etc.

The plants or fungi may have one or more parts that are improved, e.g., leaves, stems, roots, tubers, seeds, endosperm, ovule, and pollen. The parts may be viable, nonviable, regeneratable, and/or non-regeneratable.

The improved plants and fungi may include gametes, seeds, embryos, either zygotic or somatic, progeny and/or hybrids of improved plants and fungi. The progeny may be a clone of the produced plant or fungi, or may result from sexual reproduction by crossing with other individuals of the same species to introgress further desirable traits into their offspring. The cell may be in vivo or ex vivo in the cases of multicellular organisms, particularly plants.

### Further Applications in Plants

Further applications of the compositions, systems, and methods on plants and fungi include visualization of genetic element dynamics (e.g., as described in Chen B, et al., Cell. 2013 Dec. 19; 155(7):1479-91), targeted gene disruption positive-selection in vitro and in vivo (as described in Malina A et al., Genes Dev. 2013 Dec. 1; 27(23):2602-14), epigenetic modification such as using fusion of nucleic acid-guided nuclease and histone-modifying enzymes (e.g., as described in Rusk N, Nat Methods. 2014 January; 11(1):28), identifying transcription regulators (e.g., as described in Waldrip Z J, Epigenetics. 2014 September; 9(9):1207-11), anti-virus treatment for both RNA and DNA viruses (e.g., as described in Price A A, et al., Proc Natl Acad Sci USA. 2015 May 12; 112(19):6164-9; Ramanan V et al., Sci Rep. 2015 Jun. 2; 5:10833), alteration of genome complexity such as chromosome numbers (e.g., as described in Karimi-Ashtiyani R et al., Proc Natl Acad Sci USA. 2015 Sep. 8; 112(36):11211-6; Anton T, et al., Nucleus. 2014 March-April; 5(2):163-72), self-cleavage of the composition for controlled inactivation/activation (e.g., as described Sugano S S et al., Plant Cell Physiol. 2014 March; 55(3):475-81), multiplexed gene editing (as described in Kabadi A M et al., Nucleic Acids Res. 2014 Oct. 29; 42(19):e147), development of kits for multiplex genome editing (as described in Xing H L et al., BMC Plant Biol. 2014 Nov. 29; 14:327), starch production (as described in Hebelstrup K H et al., Front Plant Sci. 2015 Apr. 23; 6:247), targeting multiple genes in a family or pathway (e.g., as described in Ma X et al., Mol Plant. 2015 August; 8(8):1274-84), regulation of non-coding genes and sequences (e.g., as described in Lowder L G, et al., Plant Physiol. 2015 October; 169(2):971-85), editing genes in trees (e.g., as described in Belhaj K et al., Plant Methods. 2013 Oct. 11; 9(1):39; Harrison M M, et al., Genes Dev. 2014 Sep. 1; 28(17):1859-72; Zhou X et al., New Phytol. 2015 October; 208(2):298-301), introduction of mutations for resistance to host-specific pathogens and pests.

Additional examples of modifications of plants and fungi that may be performed using the compositions, systems, and methods include those described in International Patent Publication Nos. WO2016/099887, WO2016/025131, WO2016/073433, WO2017/066175, WO2017/100158, WO 2017/105991, WO2017/106414, WO2016/100272, WO2016/100571, WO 2016/100568, WO 2016/100562, and WO 2017/019867.

### Applications in Non-Human Animals

The compositions, systems, and methods may be used to study and modify non-human animals, e.g., introducing desirable traits and disease resilience, treating diseases, facilitating breeding, etc. In some embodiments, the compositions, systems, and methods may be used to improve breeding and introducing desired traits, e.g., increasing the frequency of trait-associated alleles, introgression of alleles from other breeds/species without linkage drag, and creation of de novo favorable alleles. Genes and other genetic elements that can be targeted may be screened and identified. Examples of application and approaches include those described in Tait-Burkard C, et al., Livestock 2.0-genome editing for fitter, healthier, and more productive farmed animals. Genome Biol. 2018 Nov. 26; 19(1):204; Lillico S, Agricultural applications of genome editing in farmed animals. Transgenic Res. 2019 August; 28(Suppl 2):57-60; Houston R D, et al., Harnessing genomics to fast-track genetic improvement in aquaculture. Nat Rev Genet. 2020 Apr. 16. doi: 10.1038/s41576-020-0227-y, which are incorporated herein by reference in their entireties. Applications described in other sections such as therapeutic, diagnostic, etc. can also be used on the animals herein.

The compositions, systems, and methods may be used on animals such as fish, amphibians, reptiles, mammals, and birds. The animals may be farm and agriculture animals, or pets. Examples of farm and agriculture animals include horses, goats, sheep, swine, cattle, llamas, alpacas, and birds, e.g., chickens, turkeys, ducks, and geese. The animals may be a non-human primate, e.g., baboons, capuchin monkeys, chimpanzees, lemurs, macaques, marmosets, tamarins, spider monkeys, squirrel monkeys, and vervet monkeys. Examples of pets include dogs, cats horses, wolfs, rabbits, ferrets, gerbils, hamsters, chinchillas, fancy rats, guinea pigs, canaries, parakeets, and parrots.

In some embodiments, one or more genes may be introduced (e.g., overexpressed) in the animals to obtain or enhance one or more desired traits. Growth hormones, insulin-like growth factors (IGF-1) may be introduced to increase the growth of the animals, e.g., pigs or salmon (such as described in Pursel V G et al., J Reprod Fertil Suppl. 1990; 40:235-45; Waltz E, Nature. 2017; 548:148). Fat-1 gene (e.g., from C elegans) may be introduced for production of larger ratio of n−3 to n−6 fatty acids may be induced, e.g. in pigs (such as described in Li M, et al., Genetics. 2018; 8:1747-54). Phytase (e.g., from E coli) xylanase (e.g., from Aspergillus niger), beta-glucanase (e.g., from Bacillus lichenformis) may be introduced to reduce the environmental impact through phosphorous and nitrogen release reduction, e.g. in pigs (such as described in Golovan S P, et al., Nat Biotechnol. 2001; 19:741-5; Zhang X et al., elife. 2018). shRNA decoy may be introduced to induce avian influenza resilience e.g. in chicken (such as described in Lyall et al., Science. 2011; 331:223-6). Lysozyme or lysostaphin may be introduced to induce mastitis resilience e.g., in goat and cow (such as described in Maga E A et al., Foodborne Pathog Dis. 2006; 3:384-92; Wall R J, et al., Nat Biotechnol. 2005; 23:445-51). Histone deacetylase such as HDAC6 may be introduced to induce PRRSV resilience, e.g., in pig (such as described in Lu T., et al., PLoS One. 2017; 12:e0169317). CD163 may be modified (e.g., inactivated or removed) to introduce PRRSV resilience in pigs (such as described in Prather R S et al., Sci Rep. 2017 Oct. 17; 7(1):13371). Similar approaches may be used to inhibit or remove viruses and bacteria (e.g., Swine Influenza Virus (SIV) strains which include influenza C and the subtypes of influenza A known as H1N1, H1N2, H2N1, H3N1, H3N2, and H2N3, as well as pneumonia, meningitis and oedema) that may be transmitted from animals to humans.

In some embodiments, one or more genes may be modified or edited for disease resistance and production traits. Myostatin (e.g., GDF8) may be modified to increase muscle growth, e.g., in cow, sheep, goat, catfish, and pig (such as described in Crispo M et al., PLoS One. 2015; 10:e0136690; Wang X, et al., Anim Genet. 2018; 49:43-51; Khalil K, et al., Sci Rep. 2017; 7:7301; Kang J-D, et al., RSC Adv. 2017; 7:12541-9). Pc POLLED may be modified to induce horlessness, e.g., in cow (such as described in Carlson D F et al., Nat Biotechnol. 2016; 34:479-81). KISS1R may be modified to induce boretaint (hormone release during sexual maturity leading to undesired meat taste), e.g., in pigs. Dead end protein (dnd) may be modified to induce sterility, e.g., in salmon (such as described in Wargelius A, et al., Sci Rep. 2016; 6:21284). Nano2 and DDX may be modified to induce sterility (e.g., in surrogate hosts), e.g., in pigs and chicken (such as described Park K-E, et al., Sci Rep. 2017; 7:40176; Taylor L et al., Development. 2017; 144:928-34). CD163 may be modified to induce PRRSV resistance, e.g., in pigs (such as described in Whitworth K M, et al., Nat Biotechnol. 2015; 34:20-2) RELA may be modified to induce ASFV resilience, e.g., in pigs (such as described in Lillico S G, et al., Sci Rep. 2016; 6:21645). CD18 may be modified to induce Mannheimia (Pasteurella) haemolytica resilience, e.g., in cows (such as described in Shanthalingam S, et al., roc Natl Acad Sci USA. 2016; 113:13186-90). NRAMP1 may be modified to induce tuberculosis resilience, e.g., in cows (such as described in Gao Y et al., Genome Biol. 2017; 18:13). Endogenous retrovirus genes may be modified or removed for xenotransplantation such as described in Yang L, et al. Science. 2015; 350:1101-4; Niu D et al., Science. 2017; 357:1303-7). Negative regulators of muscle mass (e.g., Myostatin) may be modified (e.g., inactivated) to increase muscle mass, e.g., in dogs (as described in Zou Q et al., J Mol Cell Biol. 2015 December; 7(6):580-3).

Animals such as pigs with severe combined immunodeficiency (SCID) may generated (e.g., by modifying RAG2) to provide useful models for regenerative medicine, xenotransplantation (discussed also elsewhere herein), and tumor development. Examples of methods and approaches include those described Lee K, et al., Proc Natl Acad Sci USA. 2014 May 20; 111(20):7260-5; and Schomberg et al. FASEB Journal, April 2016; 30(1): Suppl 571.1.

SNPs in the animals may be modified. Examples of methods and approaches include those described Tan W. et al., Proc Natl Acad Sci USA. 2013 Oct. 8; 110(41):16526-31; Mali P, et al., Science. 2013 Feb. 15; 339(6121):823-6.

Stem cells (e.g., induced pluripotent stem cells) may be modified and differentiated into desired progeny cells, e.g., as described in Heo Y T et al., Stem Cells Dev. 2015 Feb. 1; 24(3):393-402.

Profile analysis (such as Igenity) may be performed on animals to screen and identify genetic variations related to economic traits. The genetic variations may be modified to introduce or improve the traits, such as carcass composition, carcass quality, maternal and reproductive traits and average daily gain.

### Therapeutic Uses and Methods of Treatment

Also provided herein are methods of diagnosing, prognosing, treating, and/or preventing a disease, state, or condition in or of a subject. Generally, the methods of diagnosing, prognosing, treating, and/or preventing a disease, state, or condition in or of a subject can include modifying a polynucleotide in a subject or cell thereof using a composition, system, or component thereof described herein and/or include detecting a diseased or healthy polynucleotide in a subject or cell thereof using a composition, system, or component thereof described herein. In some embodiments, the method of treatment or prevention can include using a composition, system, or component thereof to modify a polynucleotide of an infectious organism (e.g. bacterial or virus) within a subject or cell thereof. In some embodiments, the method of treatment or prevention can include using a composition, system, or component thereof to modify a polynucleotide of an infectious organism or symbiotic organism within a subject. The composition, system, and components thereof can be used to develop models of diseases, states, or conditions. The composition, system, and components thereof can be used to detect a disease state or correction thereof, such as by a method of treatment or prevention described herein. The composition, system, and components thereof can be used to screen and select cells that can be used, for example, as treatments or preventions described herein. The composition, system, and components thereof can be used to develop biologically active agents that can be used to modify one or more biologic functions or activities in a subject or a cell thereof.

In general, the method can include delivering a composition, system, and/or component thereof to a subject or cell thereof, or to an infectious or symbiotic organism by a suitable delivery technique and/or composition. Once administered the components can operate as described elsewhere herein to elicit a nucleic acid modification event. In some aspects, the nucleic acid modification event can occur at the genomic, epigenomic, and/or transcriptomic level. DNA and/or RNA cleavage, gene activation, and/or gene deactivation can occur. Additional features, uses, and advantages are described in greater detail below. On the basis of this concept, several variations are appropriate to elicit a genomic locus event, including DNA cleavage, gene activation, or gene deactivation. Using the provided compositions, the person skilled in the art can advantageously and specifically target single or multiple loci with the same or different functional domains to elicit one or more genomic locus events. In addition to treating and/or preventing a disease in a subject, the compositions may be applied in a wide variety of methods for screening in libraries in cells and functional modeling in vivo (e.g. gene activation of lincRNA and identification of function; gain-of-function modeling; loss-of-function modeling; the use the compositions of the present disclosure to establish cell lines and transgenic animals for optimization and screening purposes).

The composition, system, and components thereof described elsewhere herein can be used to treat and/or prevent a disease, such as a genetic and/or epigenetic disease, in a subject. The composition, system, and components thereof described elsewhere herein can be used to treat and/or prevent genetic infectious diseases in a subject, such as bacterial infections, viral infections, fungal infections, parasite infections, and combinations thereof. The composition, system, and components thereof described elsewhere herein can be used to modify the composition or profile of a microbiome in a subject, which can in turn modify the health status of the subject. The composition, system, described herein can be used to modify cells ex vivo, which can then be administered to the subject whereby the modified cells can treat or prevent a disease or symptom thereof. This is also referred to in some contexts as adoptive therapy. The composition, system, described herein can be used to treat mitochondrial diseases, where the mitochondrial disease etiology involves a mutation in the mitochondrial DNA.

Also provided is a method of treating a subject, e.g., a subject in need thereof, comprising inducing gene editing by transforming the subject with the polynucleotide encoding one or more components of the composition, system, or complex or any of polynucleotides or vectors described herein and administering them to the subject. A suitable repair template may also be provided, for example delivered by a vector comprising said repair template. The repair template may be a recombination template herein. Also provided is a method of treating a subject, e.g., a subject in need thereof, comprising inducing transcriptional activation or repression of multiple target gene loci by transforming the subject with the polynucleotides or vectors described herein, wherein said polynucleotide or vector encodes or comprises one or more components of composition, system, complex or component thereof comprising multiple nucleic acid-guided nucleases. Where any treatment is occurring ex vivo, for example in a cell culture, then it will be appreciated that the term ‘subject’ may be replaced by the phrase “cell or cell culture.”

Also provided is a method of treating a subject, e.g., a subject in need thereof, comprising inducing gene editing by transforming the subject with the nucleic acid-guided nuclease(s), advantageously encoding and expressing in vivo the remaining portions of the composition, system, (e.g., RNA, guides). A suitable repair template may also be provided, for example delivered by a vector comprising said repair template. Also provided is a method of treating a subject, e.g., a subject in need thereof, comprising inducing transcriptional activation or repression by transforming the subject with the nucleic acid-guided nuclease(s) advantageously encoding and expressing in vivo the remaining portions of the composition, system, (e.g., RNA, guides); advantageously in some embodiments the nucleic acid-guided nuclease is a catalytically inactive nucleic acid-guided nuclease and includes one or more associated functional domains. Where any treatment is occurring ex vivo, for example in a cell culture, then it will be appreciated that the term ‘subject’ may be replaced by the phrase “cell or cell culture.”

One or more components of the composition and system described herein can be included in a composition, such as a pharmaceutical composition, and administered to a host individually or collectively. Alternatively, these components may be provided in a single composition for administration to a host. Administration to a host may be performed via viral vectors known to the skilled person or described herein for delivery to a host (e.g. lentiviral vector, adenoviral vector, AAV vector). As explained herein, use of different selection markers (e.g. for lentiviral gRNA selection) and concentration of gRNA (e.g. dependent on whether multiple gRNAs are used) may be advantageous for eliciting an improved effect.

Thus, also described herein are methods of inducing one or more polynucleotide modifications in a eukaryotic or prokaryotic cell or component thereof (e.g. a mitochondria) of a subject, infectious organism, and/or organism of the microbiome of the subject. The modification can include the introduction, deletion, or substitution of one or more nucleotides at a target sequence of a polynucleotide of one or more cell(s). The modification can occur in vitro, ex vivo, in situ, or in vivo.

In some embodiments, the method of treating or inhibiting a condition or a disease caused by one or more mutations in a genomic locus in a eukaryotic organism or a non-human organism can include manipulation of a target sequence within a coding, non-coding or regulatory element of said genomic locus in a target sequence in a subject or a non-human subject in need thereof comprising modifying the subject or a non-human subject by manipulation of the target sequence and wherein the condition or disease is susceptible to treatment or inhibition by manipulation of the target sequence including providing treatment comprising delivering a composition comprising the particle delivery system or the delivery system or the virus particle of any one of the above embodiment or the cell of any one of the above embodiment.

Also provided herein is the use of the particle delivery system or the delivery system or the virus particle of any one of the above embodiment or the cell of any one of the above embodiment in ex vivo or in vivo gene or genome editing; or for use in in vitro, ex vivo or in vivo gene therapy. Also provided herein are particle delivery systems, non-viral delivery systems, and/or the virus particle of any one of the above embodiments or the cell of any one of the above embodiments used in the manufacture of a medicament for in vitro, ex vivo or in vivo gene or genome editing or for use in in vitro, ex vivo or in vivo gene therapy or for use in a method of modifying an organism or a non-human organism by manipulation of a target sequence in a genomic locus associated with a disease or in a method of treating or inhibiting a condition or disease caused by one or more mutations in a genomic locus in a eukaryotic organism or a non-human organism.

In some embodiments, polynucleotide modification can include the introduction, deletion, or substitution of 1-75 nucleotides at each target sequence of said polynucleotide of said cell(s). The modification can include the introduction, deletion, or substitution of at least 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence. The modification can include the introduction, deletion, or substitution of at least 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s). The modification can include the introduction, deletion, or substitution of at least 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s). The modification can include the introduction, deletion, or substitution of at least 20, 21, 22, 23, 24, 25, 26, 27, 28, 29, 30, 35, 40, 45, 50, or 75 nucleotides at each target sequence of said cell(s). The modification can include the introduction, deletion, or substitution of at least 40, 45, 50, 75, 100, 200, 300, 400 or 500 nucleotides at each target sequence of said cell(s). The modification can include the introduction, deletion, or substitution of at least 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, or 9900 to 10000 nucleotides at each target sequence of said cell(s).

In some embodiments, the modifications can include the introduction, deletion, or substitution of nucleotides at each target sequence of said cell(s) via nucleic acid components (e.g. guide(s) RNA(s) or sgRNA(s)), such as those mediated by a composition, system, or a component thereof described elsewhere herein. In some embodiments, the modifications can include the introduction, deletion, or substitution of nucleotides at a target or random sequence of said cell(s) via a composition, system, or technique.

In some embodiments, the composition, system, or component thereof can promote Non-Homologous End-Joining (NHEJ). In some embodiments, modification of a polynucleotide by a composition, system, or a component thereof, such as a diseased polynucleotide, can include NHEJ. In some embodiments, promotion of this repair pathway by the composition, system, or a component thereof can be used to target gene or polynucleotide specific knock-outs and/or knock-ins. In some embodiments, promotion of this repair pathway by the composition, system, or a component thereof can be used to generate NHEJ-mediated indels. Nuclease-induced NHEJ can also be used to remove (e.g., delete) sequence in a gene of interest. Generally, NHEJ repairs a double-strand break in the DNA by joining together the two ends; however, generally, the original sequence is restored only if two compatible ends, exactly as they were formed by the double-strand break, are perfectly ligated. The DNA ends of the double-strand break are frequently the subject of enzymatic processing, resulting in the addition or removal of nucleotides, at one or both strands, prior to rejoining of the ends. This results in the presence of insertion and/or deletion (indel) mutations in the DNA sequence at the site of the NHEJ repair. The indel can range in size from 1-50 or more base pairs. In some embodiments thee indel can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 base pairs or more. If a double-strand break is targeted near to a short target sequence, the deletion mutations caused by the NHEJ repair often span, and therefore remove, the unwanted nucleotides. For the deletion of larger DNA segments, introducing two double-strand breaks, one on each side of the sequence, can result in NHEJ between the ends with removal of the entire intervening sequence. Both of these approaches can be used to delete specific DNA sequences.

In some embodiments, composition, system, mediated NHEJ can be used in the method to delete small sequence motifs. In some embodiments, composition, system, mediated NHEJ can be used in the method to generate NHEJ-mediate indels that can be targeted to the gene, e.g., a coding region, e.g., an early coding region of a gene of interest can be used to knockout (i.e., eliminate expression of) a gene of interest. For example, early coding region of a gene of interest includes sequence immediately following a transcription start site, within a first exon of the coding sequence, or within 500 bp of the transcription start site (e.g., less than 500, 450, 400, 350, 300, 250, 200, 150, 100 or 50 bp). In an embodiment, in which a guide RNA and nucleic acid-guided nuclease generate a double strand break for the purpose of inducing NHEJ-mediated indels, a guide RNA may be configured to position one double-strand break in close proximity to a nucleotide of the target position. In an embodiment, the cleavage site may be between 0-500 bp away from the target position (e.g., less than 500, 400, 300, 200, 100, 50, 40, 30, 25, 20, 15, 10, 9, 8, 7, 6, 5, 4, 3, 2 or 1 bp from the target position). In an embodiment, in which two guide RNAs complexing with one or more nickases induce two single strand breaks for the purpose of inducing NHEJ-mediated indels, two guide RNAs may be configured to position two single-strand breaks to provide for NHEJ repair a nucleotide of the target position.

For minimization of toxicity and off-target effect, it may be important to control the concentration of nucleic acid-guided nuclease mRNA and guide RNA delivered. Optimal concentrations of nucleic acid-guided nuclease mRNA and guide RNA can be determined by testing different concentrations in a cellular or non-human eukaryote animal model and using deep sequencing the analyze the extent of modification at potential off-target genomic loci. Alternatively, to minimize the level of toxicity and off-target effect, nickase mRNA (for example S. pyogenes Cas9 with the D10A mutation) can be delivered with a pair of guide RNAs targeting a site of interest. Guide sequences and strategies to minimize toxicity and off-target effects can be as in International Patent Publication No. WO 2014/093622 (PCT/US2013/074667); or, via mutation. Others are as described elsewhere herein.

Typically, in the context of an endogenous nucleic acid-guided nuclease, formation of a nucleic acid-guided nuclease or complex (comprising a guide sequence hybridized to a target sequence and complexed with one or more nucleic acid-guided nucleases) results in cleavage, nicking, and/or another modification of one or both strands in or near (e.g. within 1, 2, 3, 4, 5, 6, 7, 8, 9, 10, 20, 50, or more base pairs from) the target sequence. In some embodiments, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), can also form part of a composition herein, such as by hybridization along at least a portion of the tracr sequence to all or a portion of a tracr mate sequence that is operably linked to the guide sequence.

In some embodiments, a method of modifying a target polynucleotide in a cell to treat or prevent a disease can include allowing a composition, system, or component thereof to bind to the target polynucleotide, e.g., to effect cleavage, nicking, or other modification as the composition, system, is capable of said target polynucleotide, thereby modifying the target polynucleotide, wherein the composition, system, or component thereof, complex with a guide sequence, and hybridize said guide sequence to a target sequence within the target polynucleotide, wherein said guide sequence is optionally linked to a tracr mate sequence, which in turn can hybridize to a tracr sequence. In some of these embodiments, the composition, system, or component thereof can be or include a nucleic acid-guided nuclease complexed with a guide sequence. In some embodiments, modification can include cleaving or nicking one or two strands at the location of the target sequence by one or more components of the composition, system, or component thereof.

The cleavage, nicking, or other modification capable of being performed by the composition, system, can modify transcription of a target polynucleotide. In some embodiments, modification of transcription can include decreasing transcription of a target polynucleotide. In some embodiments, modification can include increasing transcription of a target polynucleotide. In some embodiments, the method includes repairing said cleaved target polynucleotide by homologous recombination with an recombination template polynucleotide, wherein said repair results in a modification such as, but not limited to, an insertion, deletion, or substitution of one or more nucleotides of said target polynucleotide. In some embodiments, said modification results in one or more amino acid changes in a protein expressed from a gene comprising the target sequence. In some embodiments, the modification imparted by the composition, system, or component thereof provides a transcript and/or protein that can correct a disease or a symptom thereof, including but not limited to, any of those described in greater detail elsewhere herein.

In some embodiments, the method of treating or preventing a disease can include delivering one or more vectors or vector systems to a cell, such as a eukaryotic or prokaryotic cell, wherein one or more vectors or vector systems include the composition, system, or component thereof. In some embodiments, the vector(s) or vector system(s) can be a viral vector or vector system, such as an AAV or lentiviral vector system, which are described in greater detail elsewhere herein. In some embodiments, the method of treating or preventing a disease can include delivering one or more viral particles, such as an AAV or lentiviral particle, containing the composition, system, or component thereof. In some embodiments, the viral particle has a tissue specific tropism. In some embodiments, the viral particle has a liver, muscle, eye, heart, pancreas, kidney, neuron, epithelial cell, endothelial cell, astrocyte, glial cell, immune cell, or red blood cell specific tropism.

It will be understood that the composition and system, according to the present disclosure as described herein, such as the composition and system, for use in the methods according to the present disclosure as described herein, may be suitably used for any type of application known for composition, system, preferably in eukaryotes. In certain aspects, the application is therapeutic, preferably therapeutic in a eukaryote organism, such as including but not limited to animals (including human), plants, algae, fungi (including yeasts), etc. Alternatively, or in addition, in certain aspects, the application may involve accomplishing or inducing one or more particular traits or characteristics, such as genotypic and/or phenotypic traits or characteristics, as also described elsewhere herein.

### Treating Diseases of the Circulatory System

In some embodiments, the composition, system, and/or component thereof described herein can be used to treat and/or prevent a circulatory system disease. Exemplary disease is provided, for example, in Tables 2 and 3. In some embodiments the plasma exosomes of Wahlgren et al. (Nucleic Acids Research, 2012, Vol. 40, No. 17 e130) can be used to deliver the composition, system, and/or component thereof described herein to the blood. In some embodiments, the circulatory system disease can be treated by using a lentivirus to deliver the composition, system, described herein to modify hematopoietic stem cells (HSCs) in vivo or ex vivo (see e.g. Drakopoulou, “Review Article, The Ongoing Challenge of Hematopoietic Stem Cell-Based Gene Therapy for β-Thalassemia,” Stem Cells International, Volume 2011, Article ID 987980, 10 pages, doi:10.4061/2011/987980, which can be adapted for use with the composition, system, herein in view of the description herein). In some embodiments, the circulatory system disorder can be treated by correcting HSCs as to the disease using a composition, system, herein or a component thereof, wherein the composition, system, optionally includes a suitable HDR repair template (see e.g. Cavazzana, “Outcomes of Gene Therapy for β-Thalassemia Major via Transplantation of Autologous Hematopoietic Stem Cells Transduced Ex Vivo with a Lentiviral βA-T87Q-Globin Vector.”; Cavazzana-Calvo, “Transfusion independence and HMGA2 activation after gene therapy of human β-thalassaemia”, Nature 467, 318-322 (16 Sep. 2010) doi:10.1038/nature09328; Nienhuis, “Development of Gene Therapy for Thalassemia, Cold Spring Harbor Perspectives in Medicine, doi: 10.1101/cshperspect.a011833 (2012), LentiGlobin BB305, a lentiviral vector containing an engineered β-globin gene (βA-T87Q); and Xie et al., “Seamless gene correction of β-thalassaemia mutations in patient-specific iPSCs using CRISPR/Cas9 and piggyback” Genome Research gr.173427.114 (2014) www.genome.org/cgi/doi/10.1101/gr.173427.114 (Cold Spring Harbor Laboratory Press; [1599] Watts, “Hematopoietic Stem Cell Expansion and Gene Therapy” Cytotherapy 13(10):1164-1171. doi:10.3109/14653249.2011.620748 (2011), which can be adapted for use with the composition, system, herein in view of the description herein). In some embodiments, iPSCs can be modified using a composition, system, described herein to correct a disease polynucleotide associated with a circulatory disease. In this regard, the teachings of Xu et al. (Sci Rep. 2015 Jul. 9; 5:12065. doi: 10.1038/srep12065) and Song et al. (Stem Cells Dev. 2015 May 1; 24(9):1053-65. doi: 10.1089/scd.2014.0347. Epub 2015 Feb. 5) with respect to modifying iPSCs can be adapted for use in view of the description herein with the composition, system, described herein.

The term “Hematopoietic Stem Cell” or “HSC” refers broadly those cells considered to be an HSC, e.g., blood cells that give rise to all the other blood cells and are derived from mesoderm; located in the red bone marrow, which is contained in the core of most bones. HSCs of the present disclosure include cells having a phenotype of hematopoietic stem cells, identified by small size, lack of lineage (lin) markers, and markers that belong to the cluster of differentiation series, like: CD34, CD38, CD90, CD133, CD105, CD45, and also c-kit, —the receptor for stem cell factor. Hematopoietic stem cells are negative for the markers that are used for detection of lineage commitment, and are, thus, called Lin−; and, during their purification by FACS, a number of up to 14 different mature blood-lineage markers, e.g., CD13 & CD33 for myeloid, CD71 for erythroid, CD19 for B cells, CD61 for megakaryocytic, etc. for humans; and, B220 (murine CD45) for B cells, Mac-1 (CD11b/CD18) for monocytes, Gr-1 for Granulocytes, Ter119 for erythroid cells, Il7Ra, CD3, CD4, CD5, CD8 for T cells, etc. Mouse HSC markers: CD34lo/−, SCA-1+, Thy1.1+/lo, CD38+, C-kit+, lin−, and Human HSC markers: CD34+, CD59+, Thy1/CD90+, CD38lo/−, C-kit/CD117+, and lin−. HSCs are identified by markers. Hence in embodiments discussed herein, the HSCs can be CD34+ cells. HSCs can also be hematopoietic stem cells that are CD34-/CD38-. Stem cells that may lack c-kit on the cell surface that are considered in the art as HSCs are within the ambit of the present disclosure, as well as CD133+ cells likewise considered HSCs in the art.

In some embodiments, the treatment or prevention for treating a circulatory system or blood disease can include modifying a human cord blood cell with any modification described herein. In some embodiments, the treatment or prevention for treating a circulatory system or blood disease can include modifying a granulocyte colony-stimulating factor-mobilized peripheral blood cell (mPB) with any modification described herein. In some embodiments, the human cord blood cell or mPB can be CD34+. In some embodiments, the cord blood cell(s) or mPB cell(s) modified can be autologous. In some embodiments, the cord blood cell(s) or mPB cell(s) can be allogenic. In addition to the modification of the disease gene(s), allogenic cells can be further modified using the composition, system, described herein to reduce the immunogenicity of the cells when delivered to the recipient. Such techniques are described elsewhere herein and e.g. Cartier, “MINI-SYMPOSIUM: X-Linked Adrenoleukodystrophypa, Hematopoietic Stem Cell Transplantation and Hematopoietic Stem Cell Gene Therapy in X-Linked Adrenoleukodystrophy,” Brain Pathology 20 (2010) 857-862, which can be adapted for use with the composition, system, herein. The modified cord blood cell(s) or mPB cell(s) can be optionally expanded in vitro. The modified cord blood cell(s) or mPB cell(s) can be derived to a subject in need thereof using any suitable delivery technique.

The compositions may be engineered to target genetic locus or loci in HSCs. In some embodiments, the nucleic acid-guided nuclease(s) can be codon-optimized for a eukaryotic cell and especially a mammalian cell, e.g., a human cell, for instance, HSC, or iPSC and sgRNA targeting a locus or loci in HSC, such as circulatory disease, can be prepared. These may be delivered via particles. The particles may be formed by the nucleic acid-guided nuclease and the gRNA being admixed. The gRNA and nucleic acid-guided nuclease mixture can be, for example, admixed with a mixture comprising or consisting essentially of or consisting of surfactant, phospholipid, biodegradable polymer, lipoprotein and alcohol, whereby particles containing the gRNA and nucleic acid-guided nuclease may be formed. The present disclosure comprehends so making particles and particles from such a method as well as uses thereof. Particles suitable delivery of the composition in the context of blood or circulatory system or HSC delivery to the blood or circulatory system are described in greater detail elsewhere herein.

In some embodiments, after ex vivo modification the HSCs or iPCS can be expanded prior to administration to the subject. Expansion of HSCs can be via any suitable method such as that described by, Lee, “Improved ex vivo expansion of adult hematopoietic stem cells by overcoming CUL4-mediated degradation of HOXB4.” Blood. 2013 May 16; 121(20):4082-9. doi: 10.1182/blood-2012-09-455204. Epub 2013 Mar. 21.

In some embodiments, the HSCs or iPSCs modified can be autologous. In some embodiments, the HSCs or iPSCs can be allogenic. In addition to the modification of the disease gene(s), allogenic cells can be further modified using the composition, system, described herein to reduce the immunogenicity of the cells when delivered to the recipient. Such techniques are described elsewhere herein and e.g. Cartier, “MINI-SYMPOSIUM: X-Linked Adrenoleukodystrophypa, Hematopoietic Stem Cell Transplantation and Hematopoietic Stem Cell Gene Therapy in X-Linked Adrenoleukodystrophy,” Brain Pathology 20 (2010) 857-862, which can be adapted for use with the composition, system, herein.

### Treating Neurological Diseases

In some embodiments, the compositions, systems, described herein can be used to treat diseases of the brain and CNS. Delivery options for the brain include encapsulation of nucleic acid-guided nuclease and guide RNA in the form of either DNA or RNA into liposomes and conjugating to molecular Trojan horses for trans-blood brain barrier (BBB) delivery. Molecular Trojan horses have been shown to be effective for delivery of B-gal expression vectors into the brain of non-human primates. The same approach can be used to delivery vectors containing nucleic acid-guided nuclease and guide RNA. For instance, Xia C F and Boado R J, Pardridge W M (“Antibody-mediated targeting of siRNA via the human insulin receptor using avidin-biotin technology.” Mol Pharm. 2009 May-June; 6(3):747-51. doi: 10.1021/mp800194) describes how delivery of short interfering RNA (siRNA) to cells in culture, and in vivo, is possible with combined use of a receptor-specific monoclonal antibody (mAb) and avidin-biotin technology. The authors also report that because the bond between the targeting mAb and the siRNA is stable with avidin-biotin technology, and RNAi effects at distant sites such as brain are observed in vivo following an intravenous administration of the targeted siRNA, the teachings of which can be adapted for use with the compositions, systems, herein. In other embodiments, an artificial virus can be generated for CNS and/or brain delivery. See e.g. Zhang et al. (Mol Ther. 2003 January; 7(1):11-8.)), the teachings of which can be adapted for use with the compositions, systems, herein.

### Treating Hearing Diseases

In some embodiments the composition and system described herein can be used to treat a hearing disease or hearing loss in one or both ears. Deafness is often caused by lost or damaged hair cells that cannot relay signals to auditory neurons. In such cases, cochlear implants may be used to respond to sound and transmit electrical signals to the nerve cells. But these neurons often degenerate and retract from the cochlea as fewer growth factors are released by impaired hair cells.

In some embodiments, the composition, system, or modified cells can be delivered to one or both ears for treating or preventing hearing disease or loss by any suitable method or technique. Suitable methods and techniques include, but are not limited to those set forth in US Patent Publication No. 20120328580 describes injection of a pharmaceutical composition into the ear (e.g., auricular administration), such as into the luminae of the cochlea (e.g., the Scala media, Sc vestibulae, and Sc tympani), e.g., using a syringe, e.g., a single-dose syringe. For example, one or more of the compounds described herein can be administered by intratympanic injection (e.g., into the middle ear), and/or injections into the outer, middle, and/or inner ear; administration in situ, via a catheter or pump (see e.g. McKenna et al., (U.S. Patent Publication No. 2006/0030837) and Jacobsen et al., (U.S. Pat. No. 7,206,639); administration in combination with a mechanical device such as a cochlear implant or a hearing aid, which is worn in the outer ear (see e.g. U.S. Patent Publication No. 2007/0093878, which provides an exemplary cochlear implant suitable for delivery of the compositions, systems, described herein to the ear). Such methods are routinely used in the art, for example, for the administration of steroids and antibiotics into human ears. Injection can be, for example, through the round window of the ear or through the cochlear capsule. Other inner ear administration methods are known in the art (see, e.g., Salt and Plontke, Drug Discovery Today, 10:1299-1306, 2005). In some embodiments, a catheter or pump can be positioned, e.g., in the ear (e.g., the outer, middle, and/or inner ear) of a patient during a surgical procedure. In some embodiments, a catheter or pump can be positioned, e.g., in the ear (e.g., the outer, middle, and/or inner ear) of a patient without the need for a surgical procedure.

In general, the cell therapy methods described in US Patent Publication No. 20120328580 can be used to promote complete or partial differentiation of a cell to or towards a mature cell type of the inner ear (e.g., a hair cell) in vitro. Cells resulting from such methods can then be transplanted or implanted into a patient in need of such treatment. The cell culture methods required to practice these methods, including methods for identifying and selecting suitable cell types, methods for promoting complete or partial differentiation of selected cells, methods for identifying complete or partially differentiated cell types, and methods for implanting complete or partially differentiated cells are described below.

Cells suitable for use in the present disclosure include, but are not limited to, cells that are capable of differentiating completely or partially into a mature cell of the inner ear, e.g., a hair cell (e.g., an inner and/or outer hair cell), when contacted, e.g., in vitro, with one or more of the compounds described herein. Exemplary cells that are capable of differentiating into a hair cell include, but are not limited to stem cells (e.g., inner ear stem cells, adult stem cells, bone marrow derived stem cells, embryonic stem cells, mesenchymal stem cells, skin stem cells, iPS cells, and fat derived stem cells), progenitor cells (e.g., inner ear progenitor cells), support cells (e.g., Deiters' cells, pillar cells, inner phalangeal cells, tectal cells and Hensen's cells), and/or germ cells. The use of stem cells for the replacement of inner ear sensory cells is described in Li et al., (U.S. Patent Publication No. 2005/0287127) and Li et al., (U.S. patent application Ser. No. 11/953,797). The use of bone marrow derived stem cells for the replacement of inner ear sensory cells is described in Edge et al., PCT/US2007/084654. iPS cells are described, e.g., at Takahashi et al., Cell, Volume 131, Issue 5, Pages 861-872 (2007); Takahashi and Yamanaka, Cell 126, 663-76 (2006); Okita et al., Nature 448, 260-262 (2007); Yu, J. et al., Science 318(5858):1917-1920 (2007); Nakagawa et al., Nat. Biotechnol. 26:101-106 (2008); and Zaehres and Scholer, Cell 131(5):834-835 (2007). Such suitable cells can be identified by analyzing (e.g., qualitatively or quantitatively) the presence of one or more tissue specific genes. For example, gene expression can be detected by detecting the protein product of one or more tissue-specific genes. Protein detection techniques involve staining proteins (e.g., using cell extracts or whole cells) using antibodies against the appropriate antigen. In this case, the appropriate antigen is the protein product of the tissue-specific gene expression. Although, in principle, a first antibody (i.e., the antibody that binds the antigen) can be labeled, it is more common (and improves the visualization) to use a second antibody directed against the first (e.g., an anti-IgG). This second antibody is conjugated either with fluorochromes, or appropriate enzymes for colorimetric reactions, or gold beads (for electron microscopy), or with the biotin-avidin system, so that the location of the primary antibody, and thus the antigen, can be recognized.

The composition and system may be delivered to the ear by direct application of pharmaceutical composition to the outer ear, with compositions modified from US Patent Publication No. 20110142917. In some embodiments the pharmaceutical composition is applied to the ear canal. Delivery to the ear may also be referred to as aural or otic delivery.

In some embodiments, the compositions, systems, or components thereof and/or vectors or vector systems can be delivered to ear via a transfection to the inner ear through the intact round window by a novel proteidic delivery technology which may be applied to the nucleic acid-targeting system of the present disclosure (see, e.g., Qi et al., Gene Therapy (2013), 1-9). About 40 μl of 10 mM RNA may be contemplated as the dosage for administration to the ear.

According to Rejali et al. (Hear Res. 2007 June; 228(1-2):180-7), cochlear implant function can be improved by good preservation of the spiral ganglion neurons, which are the target of electrical stimulation by the implant and brain derived neurotrophic factor (BDNF) has previously been shown to enhance spiral ganglion survival in experimentally deafened ears. Rejali et al. tested a modified design of the cochlear implant electrode that includes a coating of fibroblast cells transduced by a viral vector with a BDNF gene insert. To accomplish this type of ex vivo gene transfer, Rejali et al. transduced guinea pig fibroblasts with an adenovirus with a BDNF gene cassette insert, and determined that these cells secreted BDNF and then attached BDNF-secreting cells to the cochlear implant electrode via an agarose gel, and implanted the electrode in the scala tympani. Rejali et al. determined that the BDNF expressing electrodes were able to preserve significantly more spiral ganglion neurons in the basal turns of the cochlea after 48 days of implantation when compared to control electrodes and demonstrated the feasibility of combining cochlear implant therapy with ex vivo gene transfer for enhancing spiral ganglion neuron survival. Such a system may be applied to the nucleic acid-targeting system of the present disclosure for delivery to the ear.

In some embodiments, the system set forth in Mukherjea et al. (Antioxidants & Redox Signaling, Volume 13, Number 5, 2010) can be adapted for transtympanic administration of the composition, system, or component thereof to the ear. In some embodiments, a dosage of about 2 mg to about 4 mg of nucleic acid-guided nuclease for administration to a human.

In some embodiments, the system set forth in [Jung et al. (Molecular Therapy, vol. 21 no. 4, 834-841 April 2013) can be adapted for vestibular epithelial delivery of the composition, system, or component thereof to the ear. In some embodiments, a dosage of about 1 to about 30 mg of nucleic acid-guided nuclease for administration to a human.

### Treating Diseases in Non-Dividing Cells

In some embodiments, the gene or transcript to be corrected is in a non-dividing cell. Exemplary non-dividing cells are muscle cells or neurons. Non-dividing (especially non-dividing, fully differentiated) cell types present issues for gene targeting or genome engineering, for example because homologous recombination (HR) is generally suppressed in the G1 cell-cycle phase. However, while studying the mechanisms by which cells control normal DNA repair systems, Durocher discovered a previously unknown switch that keeps HR “off” in non-dividing cells and devised a strategy to toggle this switch back on. Orthwein et al. (Daniel Durocher's lab at the Mount Sinai Hospital in Ottawa, Canada) recently reported (Nature 16142, published online 9 Dec. 2015) have shown that the suppression of HR can be lifted and gene targeting successfully concluded in both kidney (293T) and osteosarcoma (U20S) cells. Tumor suppressors, BRCA1, PALB2 and BRAC2 are known to promote DNA DSB repair by HR. They found that formation of a complex of BRCA1 with PALB2-BRAC2 is governed by a ubiquitin site on PALB2, such that action on the site by an E3 ubiquitin ligase. This E3 ubiquitin ligase is composed of KEAP1 (a PALB2-interacting protein) in complex with cullin-3 (CUL3)-RBX1. PALB2 ubiquitylation suppresses its interaction with BRCA1 and is counteracted by the deubiquitylase USP11, which is itself under cell cycle control. Restoration of the BRCA1-PALB2 interaction combined with the activation of DNA-end resection is sufficient to induce homologous recombination in G1, as measured by a number of methods including a nucleic acid-guided nuclease-based gene-targeting assay directed at USP11 or KEAP1 (expressed from a pX459 vector). However, when the BRCA1-PALB2 interaction was restored in resection-competent G1 cells using either KEAP1 depletion or expression of the PALB2-KR mutant, a robust increase in gene-targeting events was detected. These teachings can be adapted for and/or applied to the compositions, systems, described herein.

Thus, reactivation of HR in cells, especially non-dividing, fully differentiated cell types is preferred, in some embodiments. In some embodiments, promotion of the BRCA1-PALB2 interaction is preferred in some embodiments. In some embodiments, the target ell is a non-dividing cell. In some embodiments, the target cell is a neuron or muscle cell. In some embodiments, the target cell is targeted in vivo. In some embodiments, the cell is in G1 and HR is suppressed. In some embodiments, use of KEAP1 depletion, for example inhibition of expression of KEAP1 activity, is preferred. KEAP1 depletion may be achieved through siRNA, for example as shown in Orthwein et al. Alternatively, expression of the PALB2-KR mutant (lacking all eight Lys residues in the BRCA1-interaction domain is preferred, either in combination with KEAP1 depletion or alone. PALB2-KR interacts with BRCA1 irrespective of cell cycle position. Thus, promotion or restoration of the BRCA1-PALB2 interaction, especially in G1 cells, is preferred in some embodiments, especially where the target cells are non-dividing, or where removal and return (ex vivo gene targeting) is problematic, for example neuron or muscle cells. KEAP1 siRNA is available from ThermoFischer. In some embodiments, a BRCA1-PALB2 complex may be delivered to the G1 cell. In some embodiments, PALB2 deubiquitylation may be promoted for example by increased expression of the deubiquitylase USP11, so it is envisaged that a construct may be provided to promote or up-regulate expression or activity of the deubiquitylase USP11.

### Treating Diseases of the Eye

In some embodiments, the disease to be treated is a disease that affects the eyes. Thus, in some embodiments, the composition, system, or component thereof described herein is delivered to one or both eyes.

The composition, system, can be used to correct ocular defects that arise from several genetic mutations further described in Genetic Diseases of the Eye, Second Edition, edited by Elias I. Traboulsi, Oxford University Press, 2012.

In some embodiments, the condition to be treated or targeted is an eye disorder. In some embodiments, the eye disorder may include glaucoma. In some embodiments, the eye disorder includes a retinal degenerative disease. In some embodiments, the retinal degenerative disease is selected from Stargardt disease, Bardet-Biedl Syndrome, Best disease, Blue Cone Monochromacy, Choroidermia, Cone-rod dystrophy, Congenital Stationary Night Blindness, Enhanced S-Cone Syndrome, Juvenile X-Linked Retinoschisis, Leber Congenital Amaurosis, Malattia Leventinesse, Norrie Disease or X-linked Familial Exudative Vitreoretinopathy, Pattern Dystrophy, Sorsby Dystrophy, Usher Syndrome, Retinitis Pigmentosa, Achromatopsia or Macular dystrophies or degeneration, Retinitis Pigmentosa, Achromatopsia, and age related macular degeneration. In some embodiments, the retinal degenerative disease is Leber Congenital Amaurosis (LCA) or Retinitis Pigmentosa. Other exemplary eye diseases are described in greater detail elsewhere herein.

In some embodiments, the composition, system, is delivered to the eye, optionally via intravitreal injection or subretinal injection. Intraocular injections may be performed with the aid of an operating microscope. For subretinal and intravitreal injections, eyes may be prolapsed by gentle digital pressure and fundi visualized using a contact lens system consisting of a drop of a coupling medium solution on the cornea covered with a glass microscope slide coverslip. For subretinal injections, the tip of a 10-mm 34-gauge needle, mounted on a 5-μl Hamilton syringe may be advanced under direct visualization through the superior equatorial sclera tangentially towards the posterior pole until the aperture of the needle was visible in the subretinal space. Then, 2 μl of vector suspension may be injected to produce a superior bullous retinal detachment, thus confirming subretinal vector administration. This approach creates a self-sealing sclerotomy allowing the vector suspension to be retained in the subretinal space until it is absorbed by the RPE, usually within 48 h of the procedure. This procedure may be repeated in the inferior hemisphere to produce an inferior retinal detachment. This technique results in the exposure of approximately 70% of neurosensory retina and RPE to the vector suspension. For intravitreal injections, the needle tip may be advanced through the sclera 1 mm posterior to the corneoscleral limbus and 2 μl of vector suspension injected into the vitreous cavity. For intracameral injections, the needle tip may be advanced through a corneoscleral limbal paracentesis, directed towards the central cornea, and 2 μl of vector suspension may be injected. For intracameral injections, the needle tip may be advanced through a corneoscleral limbal paracentesis, directed towards the central cornea, and 2 μl of vector suspension may be injected. These vectors may be injected at titers of either 1.0-1.4×10¹⁰ or 1.0-1.4×10⁹ transducing units (TU)/ml.

In some embodiments, for administration to the eye, lentiviral vectors. In some embodiments, the lentiviral vector is an equine infectious anemia virus (EIAV) vector. Exemplary EIAV vectors for eye delivery are described in Balagaan, J Gene Med 2006; 8: 275-285, Published online 21 Nov. 2005 in Wiley InterScience (www.interscience.wiley.com). DOI: 10.1002/jgm.845; Binley et al., HUMAN GENE THERAPY 23:980-991 (September 2012), which can be adapted for use with the composition, system, described herein. In some embodiments, the dosage can be 1.1×10⁵ transducing units per eye (TU/eye) in a total volume of 100 μl.

Other viral vectors can also be used for delivery to the eye, such as AAV vectors, such as those described in Campochiaro et al., Human Gene Therapy 17:167-176 (February 2006), Millington-Ward et al. (Molecular Therapy, vol. 19 no. 4, 642-649 April 2011; Dalkara et al. (Sci Transl Med 5, 189ra76 (2013)), which can be adapted for use with the composition, system, described herein. In some embodiments, the dose can range from about 10⁶ to 10^{9.5} particle units. In the context of the Millington-Ward AAV vectors, a dose of about 2×10¹¹ to about 6×10¹³ virus particles can be administered. In the context of Dalkara vectors, a dose of about 1×10¹⁵ to about 1×10¹⁶ vg/ml administered to a human.

In some embodiments, the sd-rxRNA® system of RXi Pharmaceuticals may be used/and or adapted for delivering composition, system, to the eye. In this system, a single intravitreal administration of 3 μg of sd-rxRNA results in sequence-specific reduction of PPIB mRNA levels for 14 days. The sd-rxRNA® system may be applied to the nucleic acid-targeting system of the present disclosure, contemplating a dose of about 3 to 20 mg of composition administered to a human.

In other embodiments, the methods of US Patent Publication No. 20130183282, which is directed to methods of cleaving a target sequence from the human rhodopsin gene, may also be modified to the nucleic acid-targeting system of the present disclosure.

In other embodiments, the methods of US Patent Publication No. 20130202678 for treating retinopathies and sight-threatening ophthalmologic disorders relating to delivering of the Puf-A gene (which is expressed in retinal ganglion and pigmented cells of eye tissues and displays a unique anti-apoptotic activity) to the sub-retinal or intravitreal space in the eye may be used or adapted. In particular, desirable targets are zgc:193933, prdm1a, spata2, tex10, rbb4, ddx3, zp2.2, Blimp-1 and HtrA2, all of which may be targeted by the composition, system, of the present disclosure.

Wu (Cell Stem Ce11, 13:659-62, 2013) designed a guide RNA that led Cas9 to a single base pair mutation that causes cataracts in mice, where it induced DNA cleavage. Then using either the other wild-type allele or oligos given to the zygotes repair mechanisms corrected the sequence of the broken allele and corrected the cataract-causing genetic defect in mutant mouse. This approach can be adapted to and/or applied to the compositions, systems, described herein.

US Patent Publication No. 20120159653, describes use of zinc finger nucleases to genetically modify cells, animals and proteins associated with macular degeneration (MD), the teachings of which can be applied to and/or adapted for the compositions, systems, described herein.

One aspect of US Patent Publication No. 20120159653 relates to editing of any chromosomal sequences that encode proteins associated with MD which may be applied to the nucleic acid-targeting system of the present disclosure.

### Treating Muscle Diseases and Cardiovascular Diseases

In some embodiments, the composition, system can be used to treat and/or prevent a muscle disease and associated circulatory or cardiovascular disease or disorder. The present disclosure also contemplates delivering the composition, system, described herein, e.g. Cas effector protein systems, to the heart. For the heart, a myocardium tropic adeno-associated virus (AAVM) is preferred, in particular AAVM41 which showed preferential gene transfer in the heart (see, e.g., Lin-Yanga et al., PNAS, Mar. 10, 2009, vol. 106, no. 10). Administration may be systemic or local. A dosage of about 1-10×10¹⁴ vector genomes are contemplated for systemic administration. See also, e.g., Eulalio et al. (2012) Nature 492: 376 and Somasuntharam et al. (2013) Biomaterials 34: 7790, the teachings of which can be adapted for and/or applied to the compositions, systems, described herein.

For example, US Patent Publication No. 20110023139, the teachings of which can be adapted for and/or applied to the compositions, systems, described herein describes use of zinc finger nucleases to genetically modify cells, animals and proteins associated with cardiovascular disease. Cardiovascular diseases generally include high blood pressure, heart attacks, heart failure, and stroke and TIA. Any chromosomal sequence involved in cardiovascular disease or the protein encoded by any chromosomal sequence involved in cardiovascular disease may be utilized in the methods described in this disclosure. The cardiovascular-related proteins are typically selected based on an experimental association of the cardiovascular-related protein to the development of cardiovascular disease. For example, the production rate or circulating concentration of a cardiovascular-related protein may be elevated or depressed in a population having a cardiovascular disorder relative to a population lacking the cardiovascular disorder. Differences in protein levels may be assessed using proteomic techniques including but not limited to Western blot, immunohistochemical staining, enzyme linked immunosorbent assay (ELISA), and mass spectrometry. Alternatively, the cardiovascular-related proteins may be identified by obtaining gene expression profiles of the genes encoding the proteins using genomic techniques including but not limited to DNA microarray analysis, serial analysis of gene expression (SAGE), and quantitative real-time polymerase chain reaction (Q-PCR).

The compositions, systems, herein can be used for treating diseases of the muscular system. The present disclosure also contemplates delivering the composition, system, described herein, effector protein systems, to muscle(s).

In some embodiments, the muscle disease to be treated is a muscle dystrophy such as DMD. In some embodiments, the composition, system, such as a system capable of RNA modification, described herein can be used to achieve exon skipping to achieve correction of the diseased gene. As used herein, the term “exon skipping” refers to the modification of pre-mRNA splicing by the targeting of splice donor and/or acceptor sites within a pre-mRNA with one or more complementary antisense oligonucleotide(s) (AONs). By blocking access of a spliceosome to one or more splice donor or acceptor site, an AON may prevent a splicing reaction thereby causing the deletion of one or more exons from a fully-processed mRNA. Exon skipping may be achieved in the nucleus during the maturation process of pre-mRNAs. In some examples, exon skipping may include the masking of key sequences involved in the splicing of targeted exons by using a composition, system, described herein capable of RNA modification. In some embodiments, exon skipping can be achieved in dystrophin mRNA. In some embodiments, the composition, system, can induce exon skipping at exon 1, 2, 3, 4, 5, 6, 7, 8, 10, 11, 12, 13, 14, 15, 16, 17, 18, 19, 20, 21, 22, 23, 24, 25, 26, 27, 28, 29, 30, 31, 32, 33, 34, 45, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or any combination thereof of the dystrophin mRNA. In some embodiments, the composition, system, can induce exon skipping at exon 43, 44, 50, 51, 52, 55, or any combination thereof of the dystrophin mRNA. Mutations in these exons, can also be corrected using non-exon skipping polynucleotide modification methods.

In some embodiments, for treatment of a muscle disease, the method of Bortolanza et al. Molecular Therapy vol. 19 no. 11, 2055-264 November 2011) may be applied to an AAV expressing nucleic acid-guided nuclease and injected into humans at a dosage of about 2×10¹⁵ or 2×10¹⁶ vg of vector. The teachings of Bortolanza et al., can be adapted for and/or applied to the compositions, systems, described herein.

In some embodiments, the method of Dumonceaux et al. (Molecular Therapy vol. 18 no. 5, 881-887 May 2010) may be applied to an AAV expressing nucleic acid-guided nuclease and injected into humans, for example, at a dosage of about 10¹⁴ to about 10¹⁵ vg of vector. The teachings of Dumonceaux described herein can be adapted for and/or applied to the compositions, systems, described herein.

In some embodiments, the method of Kinouchi et al. (Gene Therapy (2008) 15, 1126-1130) may be applied to compositions described herein and injected into a human, for example, at a dosage of about 500 to 1000 ml of a 40 μM solution into the muscle.

In some embodiments, the method of Hagstrom et al. (Molecular Therapy Vol. 10, No. 2, August 2004) can be adapted for and/or applied to the compositions, systems, herein and injected at a dose of about 15 to about 50 mg into the great saphenous vein of a human.

In some embodiments, the method comprise treating a sickle cell related disease, e.g., sickle cell trait, sickle cell disease such as sickle cell anemia, β-thalassaemia. For example, the method and system may be used to modify the genome of the sickle cell, e.g., by correcting one or more mutations of the β-globin gene. In the case of β-thalassaemia, sickle cell anemia can be corrected by modifying HSCs with the systems. The system allows the specific editing of the cell's genome by cutting its DNA and then letting it repair itself. The nucleic acid-guided nuclease is inserted and directed by a RNA guide to the mutated point and then it cuts the DNA at that point. Simultaneously, a healthy version of the sequence is inserted. This sequence is used by the cell's own repair system to fix the induced cut. In this way, the nucleic acid-guided nuclease allows the correction of the mutation in the previously obtained stem cells. The methods and systems may be used to correct HSCs as to sickle cell anemia using a systems that targets and corrects the mutation (e.g., with a suitable HDR template that delivers a coding sequence for β-globin, advantageously non-sickling β-globin); specifically, the guide RNA can target mutation that give rise to sickle cell anemia, and the HDR can provide coding for proper expression of β-globin. An guide RNA that targets the mutation-and-nucleic acid-guided nuclease containing particle is contacted with HSCs carrying the mutation. The particle also can contain a suitable HDR template to correct the mutation for proper expression of β-globin; or the HSC can be contacted with a second particle or a vector that contains or delivers the HDR template. The so contacted cells can be administered; and optionally treated/expanded; cf. Cartier. The HDR template can provide for the HSC to express an engineered β-globin gene (e.g., βA-T87Q), or β-globin.

### Treating Diseases of the Liver and Kidney

In some embodiments, the composition, system, or component thereof described herein can be used to treat a disease of the kidney or liver. Thus, in some embodiments, delivery of the composition or component thereof described herein is to the liver or kidney.

Delivery strategies to induce cellular uptake of the therapeutic nucleic acid include physical force or vector systems such as viral-, lipid- or complex-based delivery, or nanocarriers. From the initial applications with less possible clinical relevance, when nucleic acids were addressed to renal cells with hydrodynamic high-pressure injection systemically, a wide range of gene therapeutic viral and non-viral carriers have been applied already to target posttranscriptional events in different animal kidney disease models in vivo (Csaba Révész and Péter Hamar (2011). Delivery Methods to Target RNAs in the Kidney, Gene Therapy Applications, Prof. Chunsheng Kang (Ed.), ISBN: 978-953-307-541-9, InTech, Available from: www.intechopen.com/books/gene-therapy-applications/delivery-methods-to-target-rnas-inthe-kidney). Delivery methods to the kidney may include those in Yuan et al. (Am J Physiol Renal Physiol 295: F605-F617, 2008). The method of Yuang et al. may be applied to the composition of the present disclosure contemplating a 1-2 g subcutaneous injection of nucleic acid-guided nuclease conjugated with cholesterol to a human for delivery to the kidneys. In some embodiments, the method of Molitoris et al. (J Am Soc Nephrol 20: 1754-1764, 2009) can be adapted to the composition and a cumulative dose of 12-20 mg/kg to a human can be used for delivery to the proximal tubule cells of the kidneys. In some embodiments, the methods of Thompson et al. (Nucleic Acid Therapeutics, Volume 22, Number 4, 2012) can be adapted to the compositions and a dose of up to 25 mg/kg can be delivered via i.v. administration. In some embodiments, the method of Shimizu et al. (J Am Soc Nephrol 21: 622-633, 2010) can be adapted to the compositions and a dose of about of 10-20 μmol compositions complexed with nanocarriers in about 1-2 liters of a physiologic fluid for i.p. administration can be used.

Other various delivery vehicles can be used to deliver the composition, system to the kidney such as viral, hydrodynamic, lipid, polymer nanoparticles, aptamers and various combinations thereof (see e.g. Larson et al., Surgery, (August 2007), Vol. 142, No. 2, pp. (262-269); Hamar et al., Proc Natl Acad Sci, (October 2004), Vol. 101, No. 41, pp. (14883-14888); Zheng et al., Am J Pathol, (October 2008), Vol. 173, No. 4, pp. (973-980); Feng et al., Transplantation, (May 2009), Vol. 87, No. 9, pp. (1283-1289); Q. Zhang et al., PloS ONE, (July 2010), Vol. 5, No. 7, e11709, pp. (1-13); Kushibikia et al., J Controlled Release, (July 2005), Vol. 105, No. 3, pp. (318-331); Wang et al., Gene Therapy, (July 2006), Vol. 13, No. 14, pp. (1097-1103); Kobayashi et al., Journal of Pharmacology and Experimental Therapeutics, (February 2004), Vol. 308, No. 2, pp. (688-693); Wolfrum et al., Nature Biotechnology, (September 2007), Vol. 25, No. 10, pp. (1149-1157); Molitoris et al., J Am Soc Nephrol, (August 2009), Vol. 20, No. 8 pp. (1754-1764); Mikhaylova et al., Cancer Gene Therapy, (March 2011), Vol. 16, No. 3, pp. (217-226); Y. Zhang et al., J Am Soc Nephrol, (April 2006), Vol. 17, No. 4, pp. (1090-1101); Singhal et al., Cancer Res, (May 2009), Vol. 69, No. 10, pp. (4244-4251); Malek et al., Toxicology and Applied Pharmacology, (April 2009), Vol. 236, No. 1, pp. (97-108); Shimizu et al., J Am Soc Nephrology, (April 2010), Vol. 21, No. 4, pp. (622-633); Jiang et al., Molecular Pharmaceutics, (May-June 2009), Vol. 6, No. 3, pp. (727-737); Cao et al, J Controlled Release, (June 2010), Vol. 144, No. 2, pp. (203-212); Ninichuk et al., Am J Pathol, (March 2008), Vol. 172, No. 3, pp. (628-637); Purschke et al., Proc Natl Acad Sci, (March 2006), Vol. 103, No. 13, pp. (5173-5178).

In some embodiments, delivery is to liver cells. In some embodiments, the liver cell is a hepatocyte. Delivery of the composition and system herein may be via viral vectors, especially AAV (and in particular AAV2/6) vectors. These can be administered by intravenous injection. A preferred target for the liver, whether in vitro or in vivo, is the albumin gene. This is a so-called ‘safe harbor” as albumin is expressed at very high levels and so some reduction in the production of albumin following successful gene editing is tolerated. It is also preferred as the high levels of expression seen from the albumin promoter/enhancer allows for useful levels of correct or transgene production (from the inserted recombination template) to be achieved even if only a small fraction of hepatocytes are edited. See sites identified by Wechsler et al. (reported at the 57th Annual Meeting and Exposition of the American Society of Hematology abstract available online at ash.confex.com/ash/2015/webprogram/Paper86495.html and presented on 6th December 2015) which can be adapted for use with the compositions, systems, herein.

Exemplary liver and kidney diseases that can be treated and/or prevented are described elsewhere herein.

### Treating Epithelial and Lung Diseases

In some embodiments, the disease treated or prevented by the composition and system described herein can be a lung or epithelial disease. The compositions and systems described herein can be used for treating epithelial and/or lung diseases. The present disclosure also contemplates delivering the composition, system, described herein, to one or both lungs.

In some embodiments, as viral vector can be used to deliver the composition, system, or component thereof to the lungs. In some embodiments, the AAV is an AAV-1, AAV-2, AAV-5, AAV-6, and/or AAV-9 for delivery to the lungs. (see, e.g., Li et al., Molecular Therapy, vol. 17 no. 12, 2067-277 December 2009). In some embodiments, the MOI can vary from 1×10³ to 4×10⁵ vector genomes/cell. In some embodiments, the delivery vector can be an RSV vector as in Zamora et al. (Am J Respir Crit Care Med Vol 183. pp 531-538, 2011. The method of Zamora et al. may be applied to the nucleic acid-targeting system of the present disclosure and an aerosolized composition, for example with a dosage of 0.6 mg/kg, may be contemplated for the present disclosure.

Subjects treated for a lung disease may for example receive pharmaceutically effective amount of aerosolized AAV vector system per lung endobronchially delivered while spontaneously breathing. As such, aerosolized delivery is preferred for AAV delivery in general. An adenovirus or an AAV particle may be used for delivery. Suitable gene constructs, each operably linked to one or more regulatory sequences, may be cloned into the delivery vector. In this instance, the following constructs are provided as examples: Cbh or EF1a promoter for Cas, U6 or H1 promoter for guide RNA), A preferred arrangement is to use a CFTRdelta508 targeting guide, a repair template for deltaF508 mutation and a codon optimized composition, with optionally one or more nuclear localization signal or sequence(s) (NLS(s)), e.g., two (2) NLSs.

### Treating Diseases of the Skin

The compositions and systems described herein can be used for the treatment of skin diseases. The present disclosure also contemplates delivering the composition and system, described herein, to the skin.

In some embodiments, delivery to the skin (intradermal delivery) of the composition, system, or component thereof can be via one or more microneedles or microneedle containing device. For example, in some embodiments the device and methods of Hickerson et al. (Molecular Therapy-Nucleic Acids (2013) 2, e129) can be used and/or adapted to deliver the composition, system, described herein, for example, at a dosage of up to 300 μl of 0.1 mg/ml compositions to the skin.

In some embodiments, the methods and techniques of Leachman et al. (Molecular Therapy, vol. 18 no. 2, 442-446 February 2010) can be used and/or adapted for delivery of a compositions described herein to the skin.

In some embodiments, the methods and techniques of Zheng et al. (PNAS, Jul. 24, 2012, vol. 109, no. 30, 11975-11980) can be used and/or adapted for nanoparticle delivery of a compositions described herein to the skin. In some embodiments, as dosage of about 25 nM applied in a single application can achieve gene knockdown in the skin.

### Treating Cancer

The compositions, systems, described herein can be used for the treatment of cancer. The present disclosure also contemplates delivering the composition, system, described herein, to a cancer cell. Also, as is described elsewhere herein the compositions, systems, can be used to modify an immune cell, such as a CAR or CAR T cell, which can then in turn be used to treat and/or prevent cancer. This is also described in International Patent Publication No. WO 2015/161276, the disclosure of which is hereby incorporated by reference and described herein below.

Target genes suitable for the treatment or prophylaxis of cancer can include those set forth in Tables 2 and 3. In some embodiments, target genes for cancer treatment and prevention can also include those described in International Patent Publication No. WO 2015/048577 the disclosure of which is hereby incorporated by reference and can be adapted for and/or applied to the composition, system, described herein.

### Adoptive Cell Therapy

The compositions, systems, and components thereof described herein can be used to modify cells for an adoptive cell therapy. In an aspect of the present disclosure, methods and compositions which involve editing a target nucleic acid sequence, or modulating expression of a target nucleic acid sequence, and applications thereof in connection with cancer immunotherapy are comprehended by adapting the composition, system, of the present disclosure. In some examples, the compositions, systems, and methods may be used to modify a stem cell (e.g., induced pluripotent cell) to derive modified natural killer cells, gamma delta T cells, and alpha beta T cells, which can be used for the adoptive cell therapy. In certain examples, the compositions, systems, and methods may be used to modify modified natural killer cells, gamma delta T cells, and alpha beta T cells.

As used herein, “ACT”, “adoptive cell therapy” and “adoptive cell transfer” may be used interchangeably. In certain embodiments, Adoptive cell therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for β-thalassemia, Nat Commun. 2017 Sep. 4; 8(1):424). As used herein, the term “engraft” or “engraftment” refers to the process of cell incorporation into a tissue of interest in vivo through contact with existing cells of the tissue. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Zacharakis et al., (2018) Nat Med. 2018 June; 24(6):724-730; Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma, metastatic breast cancer and colorectal carcinoma, as well as patients with CD19-expressing hematologic malignancies (Kalos et al., (2011) Science Translational Medicine 3 (95): 95ra73). In certain embodiments, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

Aspects of the present disclosure involve the adoptive transfer of immune system cells, such as T cells, specific for selected antigens, such as tumor associated antigens or tumor specific neoantigens (see, e.g., Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144; and Rajasagi et al., 2014, Systematic identification of personal tumor-specific neoantigens in chronic lymphocytic leukemia. Blood. 2014 Jul. 17; 124(3):453-62).

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: MR1 (see, e.g., Crowther, et al., 2020, Genome-wide CRISPR-Cas9 screening reveals ubiquitous T cell cancer targeting via the monomorphic MHC class I-related protein MR1, Nature Immunology volume 21, pages 178-185), B cell maturation antigen (BCMA) (see, e.g., Friedman et al., Effective Targeting of Multiple BCMA-Expressing Hematological Malignancies by Anti-BCMA CAR T Cells, Hum Gene Ther. 2018 Mar. 8; Berdeja J G, et al. Durable clinical responses in heavily pretreated patients with relapsed/refractory multiple myeloma: updated results from a multicenter study of bb2121 anti-Bcma CAR T cell therapy. Blood. 2017; 130:740; and Mouhieddine and Ghobrial, Immunotherapy in Multiple Myeloma: The Era of CAR T Cell Therapy, Hematologist, May-June 2018, Volume 15, issue 3); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostase; Prostatic acid phosphatase (PAP); elongation factor 2 mutant (ELF2M); Insulin-like growth factor 1 receptor (IGF-1R); gplOO; BCR-ABL (breakpoint cluster region-Abelson); tyrosinase; New York esophageal squamous cell carcinoma 1 (NY-ESO-1); κ-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTA1 (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (iCE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac(2-8)aNeu5Ac(2-3)bDGalp(1-4)bDGlcp(1-1)Cer); Tn antigen (Tn Ag); Fms-Like Tyrosine Kinase 3 (FLT3); CD38; CD138; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis(Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac(2-3)bDGalp(1-4)bDGlcp(1-1)Cer); TGS5; high molecular weight-melanoma-associated antigen (HMWMAA); o-acetyl-GD2 ganglioside (OAcGD2); Folate receptor alpha; Folate receptor beta; tumor endothelial marker 1 (TEM1/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SART1, SART3); Paired box protein Pax-5 (PAXS); proacrosin binding protein sp32 (OY-TES1); lymphocyte-specific protein tyrosine kinase (LCK); A kinase anchor protein 4 (AKAP-4); synovial sarcoma, X breakpoint-1, -2, -3 or -4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRLS); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAP1 (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2, -3, 4); FBP (folate binding protein); fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); L1CAM (L1 cell adhesion molecule); MC1R (melanocortin 1 receptor); Myosin/m (myosin mutated); MUM-1, -2, -3 (melanoma ubiquitous mutated 1, 2, 3); NA88-A (NA cDNA clone of patient M88); KG2D (Natural killer group 2, member D) ligands; oncofetal antigen (h5T4); p190 minor bcr-abl (protein of 190KD bcr-abl); Pml/RARa (promyelocytic leukemia/retinoic acid receptor a); PRAME (preferentially expressed antigen of melanoma); SAGE (sarcoma antigen); TEL/AML1 (translocation Ets-family leukemia/acute myeloid leukemia 1); TPI/m (triosephosphate isomerase mutated); CD70; and any combination thereof.

In certain embodiments, an antigen to be targeted in adoptive cell therapy (such as particularly CAR or TCR T-cell therapy) of a disease (such as particularly of tumor or cancer) is a tumor-specific antigen (TSA).

In certain embodiments, an antigen to be targeted in adoptive cell therapy (such as particularly CAR or TCR T-cell therapy) of a disease (such as particularly of tumor or cancer) is a neoantigen.

In certain embodiments, an antigen to be targeted in adoptive cell therapy (such as particularly CAR or TCR T-cell therapy) of a disease (such as particularly of tumor or cancer) is a tumor-associated antigen (TAA).

In certain embodiments, an antigen to be targeted in adoptive cell therapy (such as particularly CAR or TCR T-cell therapy) of a disease (such as particularly of tumor or cancer) is a universal tumor antigen. In certain preferred embodiments, the universal tumor antigen is selected from the group consisting of: a human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (Dl), and any combinations thereof.

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: CD19, BCMA, CD70, CLL-1, MAGE A3, MAGE A6, HPV E6, HPV E7, WT1, CD22, CD171, ROR1, MUC16, and SSX2. In certain preferred embodiments, the antigen may be CD19. For example, CD19 may be targeted in hematologic malignancies, such as in lymphomas, more particularly in B-cell lymphomas, such as without limitation in diffuse large B-cell lymphoma, primary mediastinal b-cell lymphoma, transformed follicular lymphoma, marginal zone lymphoma, mantle cell lymphoma, acute lymphoblastic leukemia including adult and pediatric ALL, non-Hodgkin lymphoma, indolent non-Hodgkin lymphoma, or chronic lymphocytic leukemia. For example, BCMA may be targeted in multiple myeloma or plasma cell leukemia (see, e.g., 2018 American Association for Cancer Research (AACR) Annual meeting Poster: Allogeneic Chimeric Antigen Receptor T Cells Targeting B Cell Maturation Antigen). For example, CLL1 may be targeted in acute myeloid leukemia. For example, MAGE A3, MAGE A6, SSX2, and/or KRAS may be targeted in solid tumors. For example, HPV E6 and/or HPV E7 may be targeted in cervical cancer or head and neck cancer. For example, WT1 may be targeted in acute myeloid leukemia (AML), myelodysplastic syndromes (MDS), chronic myeloid leukemia (CML), non-small cell lung cancer, breast, pancreatic, ovarian or colorectal cancers, or mesothelioma. For example, CD22 may be targeted in B cell malignancies, including non-Hodgkin lymphoma, diffuse large B-cell lymphoma, or acute lymphoblastic leukemia. For example, CD171 may be targeted in neuroblastoma, glioblastoma, or lung, pancreatic, or ovarian cancers. For example, ROR1 may be targeted in ROR1+malignancies, including non-small cell lung cancer, triple negative breast cancer, pancreatic cancer, prostate cancer, ALL, chronic lymphocytic leukemia, or mantle cell lymphoma. For example, MUC16 may be targeted in MUC16ecto+epithelial ovarian, fallopian tube or primary peritoneal cancer. For example, CD70 may be targeted in both hematologic malignancies as well as in solid cancers such as renal cell carcinoma (RCC), gliomas (e.g., GBM), and head and neck cancers (HNSCC). CD70 is expressed in both hematologic malignancies as well as in solid cancers, while its expression in normal tissues is restricted to a subset of lymphoid cell types (see, e.g., 2018 American Association for Cancer Research (AACR) Annual meeting Poster: Allogeneic CRISPR Engineered Anti-CD70 CAR-T Cells Demonstrate Potent Preclinical Activity Against Both Solid and Hematological Cancer Cells).

Various strategies may for example be employed to genetically modify T cells by altering the specificity of the T cell receptor (TCR) for example by introducing new TCR a and β chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO 9215322).

In general, CARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CDS, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8α hinge domain and a CD8α transmembrane domain, to the transmembrane and intracellular signaling domains of either CD3t or FcRγ (scFv-CD3t or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3; see U.S. Pat. Nos. 8,911,993; 8,916,381; 8,975,071; 9,101,584; 9,102,760; 9,102,761). Third-generation CARs include a combination of costimulatory endodomains, such a CD3-chain, CD97, GDI 1a-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-iBB-CD3ζ or scFv-CD28-OX40-CD3; see U.S. Pat. Nos. 8,906,682; 8,399,645; 5,686,281; PCT Publication No. WO 2014/134165; PCT Publication No. WO 2012/079000). In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon Rib), CD79a, CD79b, Fc gamma RIIa, DAP10, and DAP12. In certain preferred embodiments, the primary signaling domain comprises a functional signaling domain of CD3t or FcRγ. In certain embodiments, the one or more costimulatory signaling domains comprise a functional signaling domain of a protein selected, each independently, from the group consisting of: CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In certain embodiments, the one or more costimulatory signaling domains comprise a functional signaling domain of a protein selected, each independently, from the group consisting of: 4-1BB, CD27, and CD28. In certain embodiments, a chimeric antigen receptor may have the design as described in U.S. Pat. No. 7,446,190, comprising an intracellular domain of CD3 chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM 006139. Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, certain embodiments employ a CAR comprising (a) a zeta chain portion comprising the intracellular domain of human CD3t chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34: 1157-1165), a portion of the human CD28 molecule, and the intracellular component of the human TCR-molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-ζ molecule. The exact sequence of the CD28 molecule included in the FMC63-28Z CAR corresponded to Genbank identifier NM_006139; the sequence included all amino acids starting with the amino acid sequence IEVMYPPPY (SEQ. I.D. No. 2) and continuing all the way to the carboxy-terminus of the protein. To encode the anti-CD19 scFv component of the vector, the authors designed a DNA sequence which was based on a portion of a previously published CAR (Cooper et al., (2003) Blood 101: 1637-1644). This sequence encoded the following components in frame from the 5′ end to the 3′ end: an XhoI site, the human granulocyte-macrophage colony-stimulating factor (GM-CSF) receptor α-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with XhoI and NotI. To form the MSGV-FMC63-28Z retroviral vector, the XhoI and NotI-digested fragment encoding the FMC63 scFv was ligated into a second XhoI and NotI-digested fragment that encoded the MSGV retroviral backbone (as in Hughes et al., (2005) Human Gene Therapy 16: 457-472) as well as part of the extracellular portion of human CD28, the entire transmembrane and cytoplasmic portion of human CD28, and the cytoplasmic portion of the human TCR-molecule (as in Maher et al., 2002) Nature Biotechnology 20: 70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in certain embodiments, cells intended for adoptive cell therapies, more particularly immunoresponsive cells such as T cells, may express the FMC63-28Z CAR as described by Kochenderfer et al. (supra). Hence, in certain embodiments, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD3ζ chain, and a costimulatory signaling region comprising a signaling domain of CD28. Preferably, the CD28 amino acid sequence is as set forth in Genbank identifier NM_006139 (sequence version 1, 2 or 3) starting with the amino acid sequence IEVMYPPPY and continuing all the way to the carboxy-terminus of the protein. Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in International Patent Publication No. WO 2015/187528. More particularly Example 1 and Table 1 of WO2015187528, incorporated by reference herein, demonstrate the generation of anti-CD19 CARs based on a fully human anti-CD19 monoclonal antibody (47G4, as described in US20100104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signaling domains (CD28-CD3ζ; 4-1BB-CD3ζ; CD27-CD3; CD28-CD27-CD3ζ, 4-1BB-CD27-CD3ζ; CD27-4-1BB-CD3ζ; CD28-CD27-FcεRI gamma chain; or CD28-FcεRT gamma chain) were disclosed. Hence, in certain embodiments, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO2015187528 and an intracellular T-cell signaling domain as set forth in Table 1 of No. WO 2015/187528. Preferably, the antigen is CD19, more preferably the antigen-binding element is an anti-CD19 scFv, even more preferably the mouse or human anti-CD19 scFv as described in Example 1 of. WO 2015/187528. In certain embodiments, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO2015187528.

By means of an example and without limitation, chimeric antigen receptor that recognizes the CD70 antigen is described in WO2012058460A2 (see also, Park et al., CD70 as a target for chimeric antigen receptor T cells in head and neck squamous cell carcinoma, Oral Oncol. 2018 March; 78:145-150; and Jin et al., CD70, a novel target of CAR T-cell therapy for gliomas, Neuro Oncol. 2018 Jan. 10; 20(1):55-65). CD70 is expressed by diffuse large B-cell and follicular lymphoma and also by the malignant cells of Hodgkins lymphoma, Waldenstrom's macroglobulinemia and multiple myeloma, and by HTLV-1- and EBV-associated malignancies. (Agathanggelou et al. Am. J. Pathol. 1995; 147: 1152-1160; Hunter et al., Blood 2004; 104:4881. 26; Lens et al., J Immunol. 2005; 174:6212-6219; Baba et al., J Virol. 2008; 82:3843-3852.) In addition, CD70 is expressed by non-hematological malignancies such as renal cell carcinoma and glioblastoma. (Junker et al., J Urol. 2005; 173:2150-2153; Chahlavi et al., Cancer Res 2005; 65:5428-5438) Physiologically, CD70 expression is transient and restricted to a subset of highly activated T, B, and dendritic cells.

By means of an example and without limitation, chimeric antigen receptor that recognizes BCMA has been described (see, e.g., US20160046724A1; WO2016014789A2; WO2017211900A1; WO2015158671A1; US20180085444A1; WO2018028647A1; US20170283504A1; and WO2013154760A1).

In certain embodiments, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In certain embodiments, the chimeric inhibitory receptor comprises an extracellular antigen-binding element (or portion or domain) configured to specifically bind to a target antigen, a transmembrane domain, and an intracellular immunosuppressive or repressive signaling domain. In certain embodiments, the second target antigen is an antigen that is not expressed on the surface of a cancer cell or infected cell or the expression of which is downregulated on a cancer cell or an infected cell. In certain embodiments, the second target antigen is an WIC-class I molecule. In certain embodiments, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its WIC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), nucleic acid-guided nuclease, or other methods that target the nucleic acids encoding specific TCRs (e.g., TCR-a and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., WO 2013/044225, WO 2016/000304, WO 2015/057834, WO 2015/057852, WO 2016/070061, U.S. Pat. No. 9,233,125, US 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US Patent Publication Nos. US 2015/0368342, US 2016/0175359, US 2015/0368360) and/or an exogenous signal, such as a small molecule drug (US 2016/0166613, Yung et al., Science, 2015), in order to elicit a T-cell response. Some CARs may also comprise a “suicide switch” to induce cell death of the CAR T-cells following treatment (Buddee et al., PLoS One, 2013) or to downregulate expression of the CAR following binding to the target antigen (International Patent Publication No. WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3t and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-y). CAR T cells of this kind may for example be used in animal models, for example to treat tumor xenografts.

In certain embodiments, ACT includes co-transferring CD4+Th1 cells and CD8+CTLs to induce a synergistic antitumor response (see, e.g., Li et al., Adoptive cell therapy with CD4+T helper 1 cells and CD8+ cytotoxic T cells enhances complete rejection of an established tumor, leading to generation of endogenous memory responses to non-targeted tumor epitopes. Clin Transl Immunology. 2017 October; 6(10): e160).

In certain embodiments, Th17 cells are transferred to a subject in need thereof. Th17 cells have been reported to directly eradicate melanoma tumors in mice to a greater extent than Th1 cells (Muranski P, et al., Tumor-specific Th17-polarized cells eradicate large established melanoma. Blood. 2008 Jul. 15; 112(2):362-73; and Martin-Orozco N, et al., T helper 17 cells promote cytotoxic T cell activation in tumor immunity. Immunity. 2009 Nov. 20; 31(5):787-98). Those studies involved an adoptive T cell transfer (ACT) therapy approach, which takes advantage of CD4+ T cells that express a TCR recognizing tyrosinase tumor antigen. Exploitation of the TCR leads to rapid expansion of Th17 populations to large numbers ex vivo for reinfusion into the autologous tumor-bearing hosts.

In certain embodiments, ACT may include autologous iPSC-based vaccines, such as irradiated iPSCs in autologous anti-tumor vaccines (see e.g., Kooreman, Nigel G. et al., Autologous iPSC-Based Vaccines Elicit Anti-tumor Responses In Vivo, Cell Stem Cell 22, 1-13, 2018, doi.org/10.1016/j.stem.2018.01.016).

Unlike T-cell receptors (TCRs) that are MHC restricted, CARs can potentially bind any cell surface-expressed antigen and can thus be more universally used to treat patients (see Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267). In certain embodiments, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs C S, Rosenberg S A. Exploiting the curative potential of adoptive T-cell therapy for cancer. Immunol Rev (2014) 257(1):56-71. doi:10.1111/imr.12132).

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

In certain embodiments, the treatment can be administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., T-cell-based immunotherapy: adoptive cell transfer and checkpoint inhibition. Cancer Immunol Res (2015) 3(10):1115-22; and Kamta et al., Advancing Cancer Therapy with Present and Emerging Immuno-Oncology Approaches. Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment can be administrated into patients undergoing an immunosuppressive treatment (e.g., glucocorticoid treatment). The cells or population of cells, may be made resistant to at least one immunosuppressive agent due to the inactivation of a gene encoding a receptor for such immunosuppressive agent. In certain embodiments, the immunosuppressive treatment provides for the selection and expansion of the immunoresponsive T cells within the patient.

In certain embodiments, the treatment can be administered before primary treatment (e.g., surgery or radiation therapy) to shrink a tumor before the primary treatment. In another embodiment, the treatment can be administered after primary treatment to remove any remaining cancer cells.

In certain embodiments, immunometabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267).

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, intrathecally, by intravenous or intralymphatic injection, or intraperitoneally. In some embodiments, the disclosed CARs may be delivered or administered into a cavity formed by the resection of tumor tissue (i.e. intracavity delivery) or directly into a tumor prior to resection (i.e. intratumoral delivery). In one embodiment, the cell compositions of the present disclosure are preferably administered by intravenous injection.

The administration of the cells or population of cells can consist of the administration of 104-109 cells per kg body weight, preferably 105 to 106 cells/kg body weight including all integer values of cell numbers within those ranges. Dosing in CART cell therapies may for example involve administration of from 106 to 109 cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the Herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; International Patent Publication WO 2011/146862; International Patent Publication WO 2014/011987; International Patent Publication WO 2013/040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2017, Multiplex genome editing to generate universal CAR T cells resistant to PD1 inhibition, Clin Cancer Res. 2017 May 1; 23(9):2255-2266. doi: 10.1158/1078-0432.CCR-16-1300. Epub 2016 Nov. 4; Qasim et al., 2017, Molecular remission of infant B-ALL after infusion of universal TALEN gene-edited CAR T cells, Sci Transl Med. 2017 Jan. 25; 9(374); Legut, et al., 2018, CRISPR-mediated TCR replacement generates superior anticancer transgenic T cells. Blood, 131(3), 311-322; and Georgiadis et al., Long Terminal Repeat CRISPR-CAR-Coupled “Universal” T Cells Mediate Potent Anti-leukemic Effects, Molecular Therapy, In Press, Corrected Proof, Available online 6 Mar. 2018). Cells may be edited using any CRISPR system and method of use thereof as described herein. The composition and systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed for example to insert or knock-in an exogenous gene, such as an exogenous gene encoding a CAR or a TCR, at a preselected locus in a cell (e.g. TRAC locus); to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more MHC constituent proteins in a cell; to activate a T cell; to modulate cells such that the cells are resistant to exhaustion or dysfunction; and/or increase the differentiation and/or proliferation of functionally exhausted or dysfunctional CD8+ T-cells (see International Patent Publication Nos. WO 2013/176915, WO 2014/059173, WO 2014/172606, WO 2014/184744, and WO 2014/191128).

In certain embodiments, editing may result in inactivation of a gene. By inactivating a gene, it is intended that the gene of interest is not expressed in a functional protein form. In a particular embodiment, the system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art. In certain embodiments, homology directed repair (HDR) is used to concurrently inactivate a gene (e.g., TRAC) and insert an endogenous TCR or CAR into the inactivated locus.

Hence, in certain embodiments, editing of cells, particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci (e.g., TRAC locus).

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543: 113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, α and β, which assemble to form a heterodimer and associates with the CD3-transducing subunits to form the T cell receptor complex present on the cell surface. Each α and β chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Hence, in certain embodiments, editing of cells, particularly cells intended for adoptive cell therapies, more particularly immunoresponsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as CRISPR/Cas system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present disclosure further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present disclosure allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In certain embodiments, editing of cells, particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

International Patent Publication No. WO 2014/172606 relates to the use of MT1 and/or MT2 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

By means of an example and without limitation, International Patent Publication No. WO 2016/196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD-L1, and/or disruption of a gene encoding PD-L1, wherein the disruption of the gene may be mediated by a gene editing nuclease, a zinc finger nuclease (ZFN), CRISPR/Cas9 and/or TALEN. WO2015142675 relates to immune effector cells comprising a CAR in combination with an agent (such as the composition or system herein) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIR1, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In certain embodiments, cells may be engineered to express a CAR, wherein expression and/or function of methylcytosine dioxygenase genes (TET1, TET2 and/or TET3) in the cells has been reduced or eliminated, (such as the composition or system herein) (for example, as described in WO201704916).

In certain embodiments, editing of cells, particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In certain embodiments, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in International Patent Publication Nos. WO 2016/011210 and WO 2017/011804).

In certain embodiments, editing of cells, particularly cells intended for adoptive cell therapies, more particularly immunoresponsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23 (9) 2255-2266 performed lentiviral delivery of CAR and electro-transfer of Cas mRNA and gRNAs targeting endogenous TCR, β-2 microglobulin (B2M) and PD1 simultaneously, to generate gene-disrupted allogeneic CART cells deficient of TCR, HLA class I molecule and PD1.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ, B2M and TCRα, B2M and TCRβ.

In certain embodiments, a cell may be multiplied edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L 1 and/or CTLA4); and (3) knock-out or knock-down expression of one or more MEW constituent proteins (for example, HLA-A, B and/or C, and/or B2M, preferably B2M).

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Immune cells may be obtained using any method known in the art. In one embodiment, allogenic T cells may be obtained from healthy subjects. In one embodiment T cells that have infiltrated a tumor are isolated. T cells may be removed during surgery. T cells may be isolated after removal of tumor tissue by biopsy. T cells may be isolated by any means known in the art. In one embodiment, T cells are obtained by apheresis. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In some embodiments, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells (PBMC), bone marrow, lymph node tissue, spleen tissue, and tumors. In certain embodiments of the present disclosure, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the present disclosure, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (e.g., CD14+ cells) may be depleted from blood preparations by a variety of methodologies, including anti-CD14 coated beads or columns, or utilization of the phagocytotic activity of these cells to facilitate removal. Accordingly, in one embodiment, the present disclosure uses paramagnetic particles of a size sufficient to be engulfed by phagocytotic monocytes. In certain embodiments, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In certain embodiments, the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×106/ml. In other embodiments, the concentration used can be from about 1×105/ml to 1×106/ml, and any integer value in between.

T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present disclosure may also be antigen-specific T cells. For example, tumor-specific T cells can be used. In certain embodiments, antigen-specific T cells can be isolated from a patient of interest, such as a patient afflicted with a cancer or an infectious disease. In one embodiment, neoepitopes are determined for a subject and T cells specific to these antigens are isolated. Antigen-specific cells for use in expansion may also be generated in vitro using any number of methods known in the art, for example, as described in U.S. Patent Publication No. US 20040224402 entitled, Generation and Isolation of Antigen-Specific T Cells, or in U.S. Pat. No. 6,040,177. Antigen-specific cells for use in the present disclosure may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g. via magnetic selection) the antigen specific cells prior to or following one or two rounds of expansion. Sorting or positively selecting antigen-specific cells can be carried out using peptide-WIC tetramers (Altman, et al., Science. 1996 Oct. 4; 274(5284):94-6). In another embodiment, the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MHC molecule of interest and any antigen of interest as described herein. Specific epitopes to be used in this context can be identified using numerous assays known in the art. For example, the ability of a polypeptide to bind to MHC class I may be evaluated indirectly by monitoring the ability to promote incorporation of 125I labeled β2-microglobulin (β2m) into WIC class I/β2m/peptide heterotrimeric complexes (see Parker et al., J. Immunol. 152:163, 1994).

In one embodiment cells are directly labeled with an epitope-specific reagent for isolation by flow cytometry followed by characterization of phenotype and TCRs. In one embodiment, T cells are isolated by contacting with T cell specific antibodies. Sorting of antigen-specific T cells, or generally any cells of the present disclosure, can be carried out using any of a variety of commercially available cell sorters, including, but not limited to, MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in patent publication Nos. WO2014133567 and WO2014133568, herein incorporated by reference in their entirety. Additionally, activated T cells can be selected for based on surface expression of CD107a.

In one embodiment of the present disclosure, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in patent publication No. WO 2003/057171, U.S. Pat. No. 8,034,334, and U.S. Patent Publication No. 2012/0244133, each of which is incorporated herein by reference.

In one embodiment, ex vivo T cell expansion can be performed by isolation of T cells and subsequent stimulation or activation followed by further expansion. In one embodiment of the present disclosure, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In certain embodiments, T cells comprising a CAR or an exogenous TCR, may be manufactured as described in International Patent Publication No. WO 2015/120096, by a method comprising enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In certain embodiments, T cells comprising a CAR or an exogenous TCR, may be manufactured as described in WO 2015/120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In certain embodiments, T cell maturation or differentiation in vitro may be delayed or inhibited by the method as described in International Patent Publication No. WO 2017/070395, comprising contacting one or more T cells from a subject in need of a T cell therapy with an AKT inhibitor (such as, e.g., one or a combination of two or more AKT inhibitors disclosed in claim 8 of WO2017070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

In certain embodiments, a patient in need of a T cell therapy may be conditioned by a method as described in International Patent Publication No. WO 2016/191756 comprising administering to the patient a dose of cyclophosphamide between 200 mg/m2/day and 2000 mg/m2/day and a dose of fludarabine between 20 mg/m2/day and 900 mg/m²/day.

### Diseases

Genetic Diseases and Diseases with a Genetic and/or Epigenetic Aspect

The compositions, systems, or components thereof can be used to treat and/or prevent a genetic disease or a disease with a genetic and/or epigenetic aspect. The genes and conditions exemplified herein are not exhaustive. In some embodiments, a method of treating and/or preventing a genetic disease can include administering a composition, system, and/or one or more components thereof to a subject, where the composition, system, and/or one or more components thereof is capable of modifying one or more copies of one or more genes associated with the genetic disease or a disease with a genetic and/or epigenetic aspect in one or more cells of the subject. In some embodiments, modifying one or more copies of one or more genes associated with a genetic disease or a disease with a genetic and/or epigenetic aspect in the subject can eliminate a genetic disease or a symptom thereof in the subject. In some embodiments, modifying one or more copies of one or more genes associated with a genetic disease or a disease with a genetic and/or epigenetic aspect in the subject can decrease the severity of a genetic disease or a symptom thereof in the subject. In some embodiments, the compositions, systems, or components thereof can modify one or more genes or polynucleotides associated with one or more diseases, including genetic diseases and/or those having a genetic aspect and/or epigenetic aspect, including but not limited to, any one or more set forth in Table 8. It will be appreciated that those diseases and associated genes listed herein are non-exhaustive and non-limiting. Further some genes play roles in the development of multiple diseases.

TABLE 8
Exemplary Genetic and Other Diseases and Associated Genes

| | | Addi- | |
|---|---|---|---|
| | Primary | tional | |
| | Tissues or | Tissues/ | |
| Disease | System | Systems | |
| Name | Affected | Affected | Genes |
| Achondro- | Bone and | | fibroblast growth factor |
| plasia | Muscle | | receptor 3 (FGFR3) |
| Achroma- | eye | | CNGA3, CNGB3, |
| topsia | | | GNAT2, PDE6C, |
| | | | PDE6H, ACHM2, ACHM3, |
| Acute | kidney | | NFkappaB, AATF, |
| Renal | | | p85alpha, FAS, |
| Injury | | | Apoptosis cascade |
| | | | elements (e.g. FASR, |
| | | | Caspase 2, 3, 4, 6, 7, 8, |
| | | | 9, 10, AKT, TNF |
| | | | alpha, IGF1, IGF1R, |
| | | | RIPK1), p53 |
| Age Related | eye | | Abcr; CCL2; CC2; |
| Macular | | | CP (ceruloplasmin); |
| Degeneration | | | Timp3; cathepsinD; |
| | | | VLDLR, CCR2 |
| AIDS | Immune | | KIR3DL1, NKAT3, |
| | System | | NKB1, AMB11, |
| | | | KIR3DS1, IFNG, |
| | | | CXCL12, SDF1 |
| Albinism | Skin, | | TYR, OCA2, |
| (including | hair, | | TYRP1, and SLC45A2, |
| oculo- | eyes, | | SLC24A5 and C10orf11 |
| cutaneous | | | |
| albinism | | | |
| (types 1-7) | | | |
| and ocular | | | |
| albinism) | | | |
| Alkaptonuria | Metab- | Tissues/ | HGD |
| | olism | organs | |
| | of | where | |
| | amino | homo- | |
| | acids | gentisic | |
| | | acid | |
| | | accu- | |
| | | mulates, | |
| | | partic- | |
| | | ularly | |
| | | cartilage | |
| | | (joints), | |
| | | heart | |
| | | valves, | |
| | | kidneys | |
| alpha-1 | Lung | Liver, | SERPINA1, those set forth in |
| antitrypsin | | skin, | WO2017165862, PiZ allele |
| deficiency | | vascular | |
| (AATD or | | system, | |
| A1AD) | | kidneys, | |
| | | GI | |
| ALS | CNS | | SOD1; ALS2; ALS3; |
| | | | ALS5; ALS7; STEX; |
| | | | FUS; TARDBP; VEGF |
| | | | (VEGF-a; VEGF-b; |
| | | | VEGF-c); |
| | | | DPP6; NEFH, PTGS1, |
| | | | SLC1A2, TNFRSF10B, |
| | | | PRPH, |
| | | | HSP90AA1, CRIA2, |
| | | | IFNG, AMPA2 |
| | | | S100B, FGF2, |
| | | | AOX1, CS, TXN, RAPHJ1, |
| | | | MAP3K5, NBEAL1, |
| | | | GPX1, ICA1L, |
| | | | RAC1, MAPT, |
| | | | ITPR2, ALS2CR4, GLS, |
| | | | ALS2CR8, CNTFR, |
| | | | ALS2CR11, FOLH1, |
| | | | FAM117B, P4HB, CNTF, |
| | | | SQSTM1, STRADB, NAIP, |
| | | | NLR, YWHAQ, SLC33A1, |
| | | | TRAK2, SCA1, |
| | | | NIF3L1, NIF3, |
| | | | PARD3B, COX8A, |
| | | | CDK15, HECW1, |
| | | | HECT, C2, WW 15, |
| | | | NOS1, MET, SOD2, |
| | | | HSPB1, NEFL, CTSB, |
| | | | ANG, HSPA8, |
| | | | RNase A, VAPB, |
| | | | VAMP, SNCA, alpha |
| | | | HGF, CAT, ACTB, |
| | | | NEFM, TH, BCL2, |
| | | | FAS, CASP3, CLU, |
| | | | SMN1, G6PD, BAX, |
| | | | HSF1, RNF19A, |
| | | | JUN, ALS2CR12, |
| | | | HSPA5, MAPK14, |
| | | | APEX1, TXNRD1, |
| | | | NOS2, TIMP1, |
| | | | CASP9, XIAP, GLG1, |
| | | | EPO, VEGFA, ELN, |
| | | | GDNF, NFE2L2, |
| | | | SLC6A3, HSPA4, |
| | | | APOE, PSMB8, |
| | | | DCTN2, TIMP3, |
| | | | KIFAP3, SLC1A1, |
| | | | SMN2, CCNC, STUB1, |
| | | | ALS2, PRDX6, |
| | | | SYP, CABIN1, CASP1, |
| | | | GART, CDK5, |
| | | | ATXN3, RTN4, |
| | | | C1QB, VEGFC, HTT, |
| | | | PARK7, XDH, GFAP, |
| | | | MAP2, CYCS, FCGR3B, |
| | | | CCS, UBL5, MMP9m |
| | | | SLC18A3, TRPM7, |
| | | | HSPB2, AKT1, |
| | | | DEERL1, CCL2, |
| | | | NGRN, GSR, TPPP3, |
| | | | APAF1, BTBD10, |
| | | | GLUD1, CXCR4, |
| | | | S:C1A3, FLT1, |
| | | | PON1, AR, LIF, ERBB3, |
| | | | :GA:S1, CD44, |
| | | | TP53, TLR3, GRIA1, |
| | | | GAPDH, AMPA, |
| | | | GRIK1, DES, CHAT, |
| | | | FLT4, CHMP2B, |
| | | | BAG1, CHRNA4, GSS, |
| | | | BAK1, KDR, |
| | | | GSTP1, OGG1, IL6 |
| Alzheimer's | Brain | | E1; CHIP; UCH; UBB; Tau; |
| Disease | | | LRP; PICALM; CLU; PS1; |
| | | | SORL1; CR1; VLDLR; |
| | | | UBA1; UBA3; |
| | | | CHIP28; AQP1; |
| | | | UCHL1; UCHL3; APP, AAA, |
| | | | CVAP, AD1, APOE, |
| | | | AD2, DCP1, ACE1, MPO, |
| | | | PACIP1, PAXIP1L, PTIP, |
| | | | A2M, BLMH, BMH, |
| | | | PSEN1, AD3, |
| | | | ALAS2, ABCA1, |
| | | | BIN1, BDNF, BTNL8, |
| | | | C1ORF49, CDH4, |
| | | | CHRNB2, CKLFSF2, |
| | | | CLEC4E, CR1L, CSF3R, |
| | | | CST3, CYP2C, |
| | | | DAPK1, ESR1, |
| | | | FCAR, FCGR3B, FFA2, |
| | | | FGA, GAB2, GALP, |
| | | | GAPDHS, GMPB, |
| | | | HP, HTR7, IDE, |
| | | | IF127, IFI6, IFIT2, |
| | | | IL1RN, IL-1RA, IL8RA, |
| | | | IL8RB, JAG1, |
| | | | KCNJ15, LRP6, |
| | | | MAPT, MARK4, |
| | | | MPHOSPH1, MTHFR, |
| | | | NBN, NCSTN, |
| | | | NIACR2, NMNAT3, |
| | | | NTM, ORM1, |
| | | | P2RY13, PBEF1, |
| | | | PCK1, PICALM, PLAU, |
| | | | PLXNC1, PRNP, |
| | | | PSEN1, PSEN2, PTPRA, |
| | | | RALGPS2, RGSL2, |
| | | | SELENBP1, |
| | | | SLC25A37, SORL1, |
| | | | Mitoferrin-1, TF, TFAM, |
| | | | TNF, TNFRSF10C, UBE1C |
| Amyloidosis | | | APOA1, APP, AAA, |
| | | | CVAP, AD1, GSN, |
| | | | FGA, LYZ, TTR, PALB |
| Amyloid | | | TTR, PALB |
| neuropathy | | | |
| Anemia | Blood | | CDAN1, CDA1, |
| | | | RPS19, DBA, PKLR, |
| | | | PK1, NT5C3, UMPH1, |
| | | | PSN1, RHAG, RH50A, |
| | | | NRAMP2, SPTB, |
| | | | ALAS2, ANH1, |
| | | | ASB, ABCB7, ABC7, ASAT |
| Angelman | Nervous | | UBE3A |
| Syndrome | system, | | |
| | brain | | |
| Attention | Brain | | PTCHD1 |
| Deficit | | | |
| Hyperactivity | | | |
| Disorder | | | |
| (ADHD) | | | |
| Autoimmune | Immune | | TNFRSF6, APT1, FAS, |
| lympho- | system | | CD95, ALPS1A |
| proliferative | | | |
| syndrome | | | |
| Autism, | Brain | | PTCHD1; Mecp2; |
| Autism | | | BZRAP1; MDGA2; |
| spectmm | | | Sema5A; Neurexin 1; |
| disorders | | | GLO1, RTT, PPMX, |
| (ASDs), | | | MRX16, RX79, |
| including | | | NLGN3, NLGN4, |
| Asperger's | | | KIAA1260, AUTSX2, |
| and a | | | FMR1, FMR2; |
| general | | | FXR1; FXR2; |
| diagnostic | | | MGLUR5, ATP10C, |
| category | | | CDH10, GRM6, |
| called | | | MGLUR6, CDH9, |
| Pervasive | | | CNTN4, NLGN2, |
| Develop- | | | CNTNAP2, SEMA5A, |
| mental | | | DHCR7, NLGN4X, |
| Disorders | | | NLGN4Y, DPP6, |
| (PDDs) | | | NLGN5, EN2, |
| | | | NRCAM, MDGA2, |
| | | | NRXN1, FMR2, |
| | | | AFF2, FOXP2, OR4M2, |
| | | | OXTR, FXR1, FXR2, |
| | | | PAH, GABRA1, |
| | | | PTEN, GABRA5, |
| | | | PTPRZ1, GABRB3, |
| | | | GABRG1, HIRIP3, |
| | | | SEZ6L2, HOXA1, |
| | | | SHANK3, IL6, |
| | | | SHBZRAP1, LAMB1, |
| | | | SLC6A4, SERT, |
| | | | MAPK3, TAS2R1, MAZ, |
| | | | TSC1, MDGA2, TSC2, |
| | | | MECP2, UBE3A, |
| | | | WNT2, see also |
| | | | 20110023145 |
| autosomal | kidney | liver | PKD1, PKD2 |
| dominant | | | |
| polycystic | | | |
| kidney | | | |
| disease | | | |
| (ADPKD)- | | | |
| (includes | | | |
| diseases | | | |
| such as von | | | |
| Hippel- | | | |
| Lindau | | | |
| disease and | | | |
| tubreous | | | |
| sclerosis | | | |
| complex | | | |
| disease) | | | |
| Autosomal | kidney | liver | PKDH1 |
| Recessive | | | |
| Polycystic | | | |
| Kidney | | | |
| Disease | | | |
| (ARPKD) | | | |
| Ataxia- | Nervous | various | ATM |
| Telangiectasia | system, | | |
| (a.k.a | immune | | |
| Louis Bar | system | | |
| syndrome) | | | |
| B-Cell | | | BCL7A, BCL7 |
| Non-Hodgkin | | | |
| Lymphoma | | | |
| Bardet- | Eye, | Liver, | ARL6, BBS1, BBS2, |
| Biedl | musculo- | ear, | BBS4, BBS5, BBS7, |
| syndrome | skeletal | gastro- | BBS9, BBS10, BBS12, |
| | system, | intestinal | CEP290, INPP5E, |
| | kidney, | system, | LZTFL1, MKKS, |
| | repro- | brain | MKS1, SDCCAG8, |
| | ductive | | TRIM32, TTC8 |
| | organs | | |
| Bare | blood | | TAPBP, TPSN, TAP2, |
| Lymphocyte | | | ABCB3, PSF2, |
| Syndrome | | | RING11, MHC2TA, |
| | | | C2TA, RFX5, |
| | | | RFXAP, RFX5 |
| Bartter's | kidney | | SLC12A1 (type I), |
| Syndrome | | | KCNJ1 (type II), |
| (types I, | | | CLCNKB (type III), |
| II, III, IVA | | | BSND (type IV A), or |
| and B, | | | both the CLCNKA |
| and V) | | | CLCNKB genes (type |
| | | | IV B), CASR (type V). |
| Becker | Muscle | | DMD, BMD, MYF6 |
| muscular | | | |
| dystrophy | | | |
| Best | eye | | VMD2 |
| Disease | | | |
| (Vitelliform | | | |
| Macular | | | |
| Dystrophy | | | |
| type 2) | | | |
| Bleeding | blood | | TBXA2R, P2RX1, P2X1 |
| Disorders | | | |
| Blue Cone | eye | | OPN1LW, OPN1MW, |
| Mono- | | | and LCR |
| chromacy | | | |
| Breast | Breast | | BRCA1, BRCA2, COX-2 |
| Cancer | tissue | | |
| Bruton's | Immune | | BTK |
| Disease (aka | system, | | |
| X-linked | specif- | | |
| Agamm- | ically | | |
| globulinemia) | B cells | | |
| Cancers (e.g., | Various | | FAS, BID, CTLA4, |
| lymphoma, | | | PDCD1, CBLB, |
| chronic | | | PTPN6, TRAC, TRBC, |
| lymphocytic | | | those described in |
| leukemia | | | WO2015048577 |
| (CLL), B | | | |
| cell acute | | | |
| lymphocytic | | | |
| leukemia | | | |
| (B-ALL), | | | |
| acute | | | |
| lympho- | | | |
| blastic | | | |
| leukemia, | | | |
| acute | | | |
| myeloid | | | |
| leukemia, | | | |
| non- | | | |
| Hodgkin's | | | |
| lymphoma | | | |
| (NHL), | | | |
| diffuse | | | |
| large cell | | | |
| lymphoma | | | |
| (DLCL), | | | |
| multiple | | | |
| myeloma, | | | |
| renal cell | | | |
| carcinoma | | | |
| (RCC), | | | |
| neuro- | | | |
| blastoma, | | | |
| colorectal | | | |
| cancer, | | | |
| breast cancer, | | | |
| ovarian | | | |
| cancer, | | | |
| melanoma, | | | |
| sarcoma, | | | |
| prostate | | | |
| cancer, | | | |
| lung cancer, | | | |
| esophageal | | | |
| cancer, | | | |
| hepato- | | | |
| cellular | | | |
| carcinoma, | | | |
| pancreatic | | | |
| cancer, | | | |
| astrocytoma, | | | |
| mesothelioma, | | | |
| head | | | |
| and neck | | | |
| cancer, and | | | |
| medullo- | | | |
| blastoma | | | |
| Cardio- | heart | Vascular | IL1B, XDH, TP53, |
| vascular | | system | PTGS, MB, IL4, |
| Diseases | | | ANGPT1, ABCGu8, |
| | | | CTSK, PTGIR, |
| | | | KCNJ11, INS, CRP, |
| | | | PDGFRB, CCNA2, |
| | | | PDGFB, KCNJ5, |
| | | | KCNN3, CAPN10, |
| | | | ADRA2B, ABCG5, |
| | | | PRDX2, CPAN5, |
| | | | PARP14, MEX3C, |
| | | | ACE, RNF, IL6, TNF, |
| | | | STN, SERPINE1, |
| | | | ALB, ADIPOQ, APOB, |
| | | | APOE, LEP, MTHFR, |
| | | | APOA1, EDN1, |
| | | | NPPB, NOS3, PPARG, |
| | | | PLAT, PTGS2, CETP, |
| | | | AGTR1, HMGCR, |
| | | | IGF1, SELE, |
| | | | REN, PPARA, PON1, |
| | | | KNG1, CCL2, LPL, |
| | | | VWF, F2, ICAM1, |
| | | | TGFB, NPPA, IL10, |
| | | | EPO, SOD1, VCAM1, |
| | | | IFNG, LPA, MPO, |
| | | | ESR1, MAPK, HP, |
| | | | F3, CST3, COG2, |
| | | | MMP9, SERPINC1, |
| | | | F8, HMOX1, APOC3, |
| | | | IL8, PROL1, CBS, |
| | | | NOS2, TLR4, SELP, |
| | | | ABCA1, AGT, LDLR, |
| | | | GPT, VEGFA, |
| | | | NR3C2, IL18, NOS1, |
| | | | NR3C1, FGB, HGF, |
| | | | IL1A, AKT1, LIPC, |
| | | | HSPD1, MAPK14, |
| | | | SPP1, ITGB3, CAT, |
| | | | UTS2, THBD, F10, |
| | | | CP, TNFRSF11B, |
| | | | EGFR, MMP2, PLG, |
| | | | NPY, RHOD, MAPK8, |
| | | | MYC, FN1, |
| | | | CMA1, PLAU, GNB3, |
| | | | ADRB2, SOD2, F5, |
| | | | VDR, ALOX5, |
| | | | HLA-DRB1, PARP1, |
| | | | CD40LG, PON2, |
| | | | AGER, IRS1, PTGS1, |
| | | | ECE1, F7, IRMN, |
| | | | EPHX2, IGFBP1, |
| | | | MAPK10, FAS, |
| | | | ABCB1, JUN, IGFBP3, |
| | | | CD14, PDE5A, |
| | | | AGTR2, CD40, LCAT, |
| | | | CCR5, MMP1, |
| | | | TIMP1, ADM, DYT10, |
| | | | STAT3, MMP3, |
| | | | ELN, USF1, CFH, |
| | | | HSPA4, MMP12, |
| | | | MME, F2R, SELL, |
| | | | CTSB, ANXA5, |
| | | | ADRB1, CYBA, FGA, |
| | | | GGT1, LIPG, HIF1A, |
| | | | CXCR4, PROC, |
| | | | SCARB1, CD79A, |
| | | | PLTP, ADD1, FGG, |
| | | | SAA1, KCNH2, |
| | | | DPP4, NPRI, VTN, |
| | | | KIAA0101, FOS, |
| | | | TLR2, PPIG, IL1R1, AR, |
| | | | CYP1A1, SERPINA1, |
| | | | MTR, RBP4, APOA4, |
| | | | CDKN2A, FGF2, EDNRB, |
| | | | ITGA2, VLA-2, |
| | | | CABIN1, SHBG, |
| | | | HMGB1, HSP90B2P, |
| | | | CYP3A4, GJA1, CAV1, |
| | | | ESR2, LTA, GDF15, BDNF, |
| | | | CYP2D6, NGF, SP1, |
| | | | TGIF1, SRC, EGF, |
| | | | PIK3CG, HLA-A, |
| | | | KCNQ1, CNR1, FBN1, |
| | | | CHKA, BEST1, |
| | | | CTNNB1, IL2, CD36, |
| | | | PRKAB1, TPO, |
| | | | ALDH7A1, CX3CR1, TH, |
| | | | F9, CH1, TF, HFE, |
| | | | IL17A, PTEN, GSTM1, |
| | | | DMD, GATA4, |
| | | | F13A1, TTR, FABP4, |
| | | | PON3, APOC1, INSR, |
| | | | TNFRSF1B, HTR2A, CSF3, |
| | | | CYP2C9, TXN, CYP11B2, |
| | | | PTH, CSF2, KDR, |
| | | | PLA2G2A, THBS1, |
| | | | GCG, RHOA, ALDH2, |
| | | | TCF7L2, NFE2L2, NOTCH1, |
| | | | UGT1A1, IFNA1, PPARD, |
| | | | SIRT11, GNHR1, PAPPA, |
| | | | ARR3, NPPC, AHSP, |
| | | | PTK2, IL13, MTOR, ITGB2, |
| | | | GSTT1, IL6ST, CPB2, |
| | | | CYP1A2, HNF4A, SLC64A, |
| | | | PLA2G6, TNFSF11, SLC8A1, |
| | | | F2RL1, AKR1A1, |
| | | | ALDH9A1, BGLAP, MTTP, |
| | | | MTRR, SULT1A3, RAGE, |
| | | | C4B, P2RY12, RNLS, |
| | | | CREB1, POMC, RAC1, |
| | | | LMNA, CD59, SCM5A, |
| | | | CYP1B1, MIF, MMP13, |
| | | | TIMP2, CYP19A1, CUP21A2, |
| | | | PTPN22, MYH14, |
| | | | MBL2, SELPLG, AOC3, |
| | | | CTSL1, PCNA, IGF2, ITGB1, |
| | | | CAST, CXCL12, |
| | | | IGHE, KCNE1, TFRC, |
| | | | COL1A1, COL1A2, |
| | | | IL2RB, PLA2G10, |
| | | | ANGPT2, PROCR, |
| | | | NOX4, HAMP, |
| | | | PTPN11, SLCA1, |
| | | | IL2RA, CCL5, IRF1, |
| | | | CF:AR, CA:CA, |
| | | | EIF4E, GSTP1, JAK2, |
| | | | CYP3A5, HSPG2, |
| | | | CCL3, MYD88, VIP, |
| | | | SOAT1, ADRBK1, |
| | | | NR4A2, MMP8, |
| | | | NPR2, GCH1, EPRS, |
| | | | PPARGC1A, F12, |
| | | | PECAM1, CCL4, |
| | | | CERPINA34, CASR, |
| | | | FABP2, TTF2, PROS1, |
| | | | CTF1, SGCB, YME1L1, |
| | | | CAMP, ZC3H12A, AKR1B1, |
| | | | MMP7, AHR, CSF1, |
| | | | HDAC9, CTGF, |
| | | | KCNMA1, UGT1A, |
| | | | PRKCA, COMT, |
| | | | S100B, EGR1, PRL, |
| | | | IL15, DRD4, CAMK2G, |
| | | | SLC22A2, CCL11, PGF, |
| | | | THPO, GP6, TACR1, |
| | | | NTS, HNF1A, SST, |
| | | | KCDN1, LOC646627, |
| | | | TBXAS1, CUP2J2, |
| | | | TBXA2R, ADH1C, |
| | | | ALOX12, AHSG, |
| | | | BHMT, GJA4, SLC25A4, |
| | | | ACLY, ALOX5AP, |
| | | | NUMA1, CYP27B1, |
| | | | CYSLTR2, SOD3, |
| | | | LTC4S, UCN, GHRL, |
| | | | APOC2, CLEC4A, |
| | | | KBTBD10, TNC, |
| | | | TYMS, SHC1, LRP1, |
| | | | SOCS3, ADH1B, KLK3, |
| | | | HSD11B1, VKORC1, |
| | | | SERPINB2, |
| | | | TNS1, RNF19A, EPOR, |
| | | | ITGAM, PITX2, |
| | | | MAPK7, FCGR3A, |
| | | | LEEPR, ENG, GPX1, |
| | | | GOT2, HRH1, NR112, |
| | | | CRH, HTR1A, VDAC1, |
| | | | HPSE, SFTPD, TAP2, |
| | | | RMF123, PTK2Bm NTRK2, |
| | | | IL6R, ACHE, GLP1R, |
| | | | GHR, GSR, NQO1, |
| | | | NR5A1, GJB2, |
| | | | SLC9A1, MAOA, |
| | | | PCSK9, FCGR2A, |
| | | | SERPINF1, EDN3, |
| | | | UCP2, TFAP2A, |
| | | | C4BPA, SERPINF2, |
| | | | TYMP, ALPP, CXCR2, |
| | | | SLC3A3, ABCG2, |
| | | | ADA, JAK3, |
| | | | HSPA1A, FASN, |
| | | | FGF1, F11, ATP7A, |
| | | | CR1, GFPA, ROCK1, |
| | | | MECP2, MYLK, |
| | | | BCHE, LIPE, |
| | | | ADORA1, WRN, CXCR3, |
| | | | CD81, SMAD7, |
| | | | LAMC2, MAP3K5, |
| | | | CHGA, IAPP, |
| | | | RHO, ENPP1, PTHLH, |
| | | | NRG1, VEGFC, |
| | | | ENPEP, CEBPB, |
| | | | NAGLU,. F2RL3, |
| | | | CX3CL1, BDKRB1, |
| | | | ADAMTS13, ELANE, |
| | | | ENPP2, CISH, |
| | | | GAST, MYOC, |
| | | | ATP1A2, NF1, GJB1, |
| | | | MEF2A, VCL, BMPR2, |
| | | | TUBB, CDC42, |
| | | | KRT18, HSF1, |
| | | | MYB, PRKAA2, ROCK2, |
| | | | TFP1, PRKG1, BMP2, |
| | | | CTNND1, CTH, |
| | | | CTSS, VAV2, NPY2R, |
| | | | IGFBP2, CD28, |
| | | | GSTA1, PPIA, |
| | | | APOH, S100A8, IL11, |
| | | | ALOX15, FBLN1, |
| | | | NR1H3, SCD, GIP, |
| | | | CHGB, PRKCB, |
| | | | SRD5A1, HSD11B2, |
| | | | CALCRL, GALNT2, |
| | | | ANGPTL4, KCNN4, |
| | | | PIK3C2A, HBEGF, |
| | | | CYP7A1, HLA-DRB5, |
| | | | BNIP3, GCKR, |
| | | | S100A12, PADI4, |
| | | | HSPA14, CXCR1, |
| | | | H19, KRTAP19-3, |
| | | | IDDM2, RAC2, YRY1, |
| | | | CLOCK, NGFR, |
| | | | DBH, CHRNA4, |
| | | | CACNA1C, PRKAG2, |
| | | | CHAT, PTGDS, |
| | | | NR1H2, TEK, VEGFB, |
| | | | MEF2C, MAPKAPK2, |
| | | | TNFRSF11A, HSPA9, |
| | | | CYSLTR1, MAT1A, OPRL1, |
| | | | IMPA1, CLCN2, DLD, |
| | | | PSMA6, PSMB8, |
| | | | CHI3L1, ALDH1B1, |
| | | | PARP2, STAR, LBP, |
| | | | ABCC6, RGS2, EFNB2, |
| | | | GJB6, APOA2, |
| | | | AMPD1, DYSF, |
| | | | FDFT1, EMD2, CCR6, |
| | | | GJB3, IL1RL1, ENTPD1, |
| | | | BBS4, CELSR2, |
| | | | F11R, RAPGEF3, |
| | | | HYAL1, ZNF259, |
| | | | ATOX1, ATF6, KHK, |
| | | | SAT1, GGH, |
| | | | TIMP4, SLC4A4, |
| | | | PDE2A, PDE3B, |
| | | | FADS1, FADS2, |
| | | | TMSB4X, TXNIP, |
| | | | LIMS1, RHOB, |
| | | | LY96, FOXO1, |
| | | | PNPLA2, TRH, |
| | | | GJC1, S:C17A5, FTO, |
| | | | GJD2, PRSC1, |
| | | | CASP12, GPBAR1, PXK, |
| | | | IL33, TRIB1, PBX4, |
| | | | NUPR1, 15-SEP, |
| | | | CILP2, TERC, |
| | | | GGT2, MTCO1, UOX, |
| | | | AVP |
| Cataract | eye | | CRYAA, CRYA1, |
| | | | CRYBB2, CRYB2, |
| | | | PITX3, BFSP2, |
| | | | CP49, CP47, CRYAA, |
| | | | CRYA1, PAX6, |
| | | | AN2, MGDA, CRYBA1, |
| | | | CRYB1, CRYGC, |
| | | | CRYG3, CCL, LIM2, |
| | | | MP19, CRYGD, CRYG4, |
| | | | BFSP2, CP49, |
| | | | CP47, HSF4, CTM, |
| | | | HSF4, CTM, MIP, |
| | | | AQP0, CRYAB, |
| | | | CRYA2, CTPP2, |
| | | | CRYBB1, CRYGD, |
| | | | CRYG4, CRYBB2, |
| | | | CRYB2, CRYGC, |
| | | | CRYG3, CCL, CRYAA, |
| | | | CRYA1, GJA8, |
| | | | CX50, CAE1, GJA3, |
| | | | CX46, CZP3, CAE3, |
| | | | CCM1, CAM, KRIT1 |
| CDKL-5 | Brain, | | CDKL5 |
| Deficiencies | CNS | | |
| or Mediated | | | |
| Diseases | | | |
| Charcot- | Nervous | Muscles | PMP22 (CMT1A and |
| Marie-Tooth | system | (dys- | E), MPZ (CMT1B), |
| (CMT) | | trophy) | LITAF (CMT1C), |
| disease | | | EGR2 (CMT1D), NEFL |
| (Types | | | (CMT1F), GJB1 |
| 1, 2, 3, 4,) | | | (CMT1X), MFN2 |
| | | | (CMT2A), KIF1B |
| | | | (CMT2A2B), RAB7A |
| | | | (CMT2B), TRPV4 |
| | | | (CMT2C), GARS |
| | | | (CMT2D), NEFL |
| | | | (CMT2E), GAPD1 |
| | | | (CMT2K), HSPB8 |
| | | | (CMT2L), DYNC1H1, |
| | | | CMT2O), LRSAM1 |
| | | | (CMT2P), IGHMBP2 |
| | | | (CMT2S), MORC2 |
| | | | (CMT2Z), GDAP1 |
| | | | (CMT4A), MTMR2 |
| | | | or SBF2/MTMR13 |
| | | | (CMT4B), SH3TC2 |
| | | | (CMT4C), NDRG1 |
| | | | (CMT4D), PRX |
| | | | (CMT4F), FIG4 (CMT4J), |
| | | | NT-3 |
| Chédiak- | Immune | Skin, | LYST |
| Higashi | system | hair, | |
| Syndrome | | eyes, | |
| | | neurons | |
| Choroidermia | | | CHM, REP1, |
| Chorioretinal | eye | | PRDM13, RGR, TEAD1 |
| atrophy | | | |
| Chronic | Immune | | CYBA, CYBB, |
| Granu- | system | | NCF1, NCF2, NCF4 |
| lomatous | | | |
| Disease | | | |
| Chronic | Immune | | AIRE, CARD9, |
| Muco- | system | | CLEC7A IL12B, IL12B1, |
| cutaneous | | | IL1F, IL17RA, IL17RC, |
| Candidiasis | | | RORC, STAT1, |
| | | | STAT3, TRAF31P2 |
| Cirrhosis | liver | | KRT18, KRT8, |
| | | | CIRH1A, NAIC, TEX292, |
| | | | KIAA1988 |
| Colon cancer | Gastro- | | FAP: APC HNPCC: |
| (Familial | intestinal | | MSH2, MLH1, |
| adenomatous | | | PMS2, SH6, PMS1 |
| polyposis | | | |
| (FAP) and | | | |
| hereditary | | | |
| nonpolyposis | | | |
| colon | | | |
| cancer | | | |
| (HNPCC)) | | | |
| Combined | Immune | | IL2RG, SCIDX1, |
| Immuno- | System | | SCIDX, IMD4); HIV-1 |
| deficiency | | | (CCL5, SCYA5, |
| | | | D17S136E, TCP228 |
| Cone(-rod) | eye | | AIPL1, CRX, GUA1A, |
| dystrophy | | | GUCY2D, PITPM3, |
| | | | PROM1, PRPH2, RIMS1, |
| | | | SEMA4A, ABCA4, |
| | | | ADAM9, ATF6, |
| | | | C21ORF2, C8ORF37, |
| | | | CACNA2D4, CDHR1, |
| | | | CERKL, CNGA3, CNGB3, |
| | | | CNNM4, CNAT2, |
| | | | IFT81, KCNV2, PDE6C, |
| | | | PDE6H, POC1B, RAX2, |
| | | | RDH5, RPGRIP1, |
| | | | TTLL5, RetCG1, GUCY2E |
| Congenital | eye | | CABP4, CACNA1F, |
| Stationary | | | CACNA2D4, |
| Night | | | GNAT1, CPR179, |
| Blindness | | | GRK1, GRM6, LRIT3, |
| | | | NYX, PDE6B, RDH5, |
| | | | RHO, RLBP1, |
| | | | RPE65, SAG, SLC24A1, |
| | | | TRPM1, |
| Congenital | Metab- | | ALDOB |
| Fructose | olism | | |
| Intolerance | | | |
| Cori's Disease | Various- | | AGL |
| (Glycogen | wherever | | |
| Storage | glycogen | | |
| Disease | accu- | | |
| Type III) | mulates, | | |
| | partic- | | |
| | ularly | | |
| | liver, | | |
| | heart, | | |
| | skeletal | | |
| | muscle | | |
| Corneal | eye | | APOA1, TGFBI, |
| clouding and | | | CSD2, CDGG1, CSD, |
| dystrophy | | | BIGH3, CDG2, |
| | | | TACSTD2, TROP2, M1S1, |
| | | | VSX1, RINX, PPCD, |
| | | | PPD, KTCN, COL8A2, |
| | | | FECD, PPCD2, PIP5K3, CFD |
| Cornea plana | | | KERA, CNA2 |
| congenital | | | |
| Cri du chat | | | Deletions involving |
| Syndrome, | | | only band 5p15.2 to the |
| also known | | | entire short arm |
| as 5p | | | of chromosome 5, e.g. |
| syndrome | | | CTNND2, TERT, |
| and cat cry | | | |
| syndrome | | | |
| Cystic | Lungs and | Pancreas, | CTFR, ABCC7, |
| Fibrosis (CF) | respiratory | liver, | CF, MRP7, SCNN1A, |
| | system | digestive | those described in |
| | | system, | WO2015157070 |
| | | repro- | |
| | | ductive | |
| | | system, | |
| | | exocrine, | |
| | | glands, | |
| Diabetic | kidney | | Gremlin, 12/15- |
| nephropathy | | | lipoxygenase, TIM44, |
| Dent Disease | Kidney | | Type 1: CLCN5, |
| (Types 1 | | | Type 2: ORCL |
| and 2) | | | |
| Dentatorubro- | CNS, | | Atrophin-1 and Atn1 |
| Pallidoluysian | brain, | | |
| Atrophy | muscle | | |
| (DRPLA) | | | |
| (aka Haw | | | |
| River and | | | |
| Naito- | | | |
| Oyanagi | | | |
| Disease) | | | |
| Down | various | | Chromosome 21 trisomy |
| Syndrome | | | |
| Drug | Brain | | Prkce; Drd2; Drd4; |
| Addiction | | | ABAT; GRIA2; Grm5; |
| | | | Grin1; Htr1b; Grin2a; |
| | | | Drd3; Pdyn; Gria1 |
| Duane | eye | | CHN1, indels on |
| syndrome | | | chromosomes 4 and 8 |
| (Types 1, | | | |
| 2, and 3, | | | |
| including | | | |
| subgroups A, | | | |
| B and C). | | | |
| Other names | | | |
| for this | | | |
| condition | | | |
| include: | | | |
| Duane's | | | |
| Retraction | | | |
| Syndrome | | | |
| (or DR | | | |
| syndrome), | | | |
| Eye | | | |
| Retraction | | | |
| Syndrome, | | | |
| Retraction | | | |
| Syndrome, | | | |
| Congenital | | | |
| retraction | | | |
| syndrome | | | |
| and Stilling- | | | |
| Turk-Duane | | | |
| Syndrome | | | |
| Duchenne | muscle | Cardio- | DMD, BMD, |
| muscular | | vascular, | dystrophin gene, intron |
| dystrophy | | respi- | flanking exon 51 of |
| (DMD) | | ratory | DMD gene, exon 51 |
| | | | mutations in DMD |
| | | | gene, see also |
| | | | WO2013163628 |
| | | | and US Pat. Pub. |
| | | | 20130145487 |
| Edward's | | | Complete or partial |
| Syndrome | | | trisomy of chromosome |
| (Trisomy 18) | | | 18 |
| Ehlers-Danlos | Various | | COL5A1, COL5A2, |
| Syndrome | depending | | COL1A1, COL3A1, |
| (Types I-VI) | on type: | | TNXB, PLOD1, |
| | including | | COL1A2, FKBP14 and |
| | musculo- | | ADAMTS2 |
| | skeletal, | | |
| | eye, | | |
| | vas- | | |
| | culature, | | |
| | immune, | | |
| | and skin | | |
| Emery- | muscle | | LMNA, LMN1, |
| Dreifuss | | | EMD2, FPLD, CMD1A, |
| muscular | | | HGPS, LGMD1B, |
| dystrophy | | | LMNA, LMN1, EMD2, |
| | | | FPLD, CMD1A |
| Enhanced | eye | | NR2E3, NRL |
| S-Cone | | | |
| Syndrome | | | |
| Fabry's | Various- | | GLA |
| Disease | including | | |
| | skin, | | |
| | eyes, and | | |
| | gastro- | | |
| | intestinal | | |
| | system, | | |
| | kidney, | | |
| | heart, | | |
| | brain, | | |
| | nervous | | |
| | system | | |
| Facioscapu- | muscles | | FSHMD1A, |
| lohumeral | | | FSHD1A, FRG1, |
| muscular | | | |
| dystrophy | | | |
| Factor H | blood | | HF1, CFH, HUS |
| and Factor H- | | | |
| like 1 | | | |
| Factor | blood | | Factor V (F5) |
| V Leiden | | | |
| thrombo- | | | |
| philia and | | | |
| Factor V | | | |
| deficiency | | | |
| Factor V | blood | | MCFD2 |
| and Factor | | | |
| VII deficiency | | | |
| Factor VII | blood | | F7 |
| deficiency | | | |
| Factor X | blood | | F10 |
| deficiency | | | |
| Factor XI | blood | | F11 |
| deficiency | | | |
| Factor XII | blood | | F12, HAF |
| deficiency | | | |
| Factor XIIIA | blood | | F13A1, F13A |
| deficiency | | | |
| Factor XIIIB | blood | | F13B |
| deficiency | | | |
| Familial | Cardio- | | APOB, LDLR, PCSK9 |
| Hyper- | vascular | | |
| cholester- | system | | |
| olemia | | | |
| Familial | Various- | Heart, | MEFV |
| Mediterranean | organs/ | kidney, | |
| Fever | tissues | brain/ | |
| (FMF) also | with | CNS, | |
| called | serous or | repro- | |
| recurrent | synovial | ductive | |
| polyserositis | mem- | organs | |
| or familial | branes, | | |
| paroxysmal | skin, joints | | |
| polyserositis | | | |
| Fanconi | Various- | | FANCA, FACA, FA1, |
| Anemia | blood | | FA, FAA, FAAP95, |
| | (anemia), | | FAAP90, FLJ34064, |
| | immune | | FANCC, FANCG, |
| | system, | | RAD51, BRCA1, |
| | cognitive, | | BRCA2, BRIP1, BACH1, |
| | kidneys, | | FANCJ, FANCB, FANCD1, |
| | eyes, | | FANCD2, FANCD, |
| | musculo- | | FAD, FANCE, FACE, |
| | skeletal | | FANCF, FANCI, |
| | | | ERCC4, FANCL, FANCM, |
| | | | PALB2, RAD51C, SLX4, |
| | | | UBE2T, FANCB, XRCC9, |
| | | | PHF9, KIAA1596 |
| Fanconi | kidneys | | FRTS1, GATM |
| Syndrome | | | |
| Types I | | | |
| (Childhood | | | |
| onset) and | | | |
| II (Adult | | | |
| Onset) | | | |
| Fragile X | brain | | FMR1, FMR2; FXR1; |
| syndrome | | | FXR2; mGLUR5 |
| and related | | | |
| disorders | | | |
| Fragile XE | Brain, | | FMR1 |
| Mental | nervous | | |
| Retardation | system | | |
| (aka | | | |
| Martin Bell | | | |
| syndrome) | | | |
| Friedreich | Brain, | heart | FXN/X25 |
| Ataxia | nervous | | |
| (FRDA) | system | | |
| Fuchs | Eye | | TCF4; COL8A2 |
| endothelial | | | |
| corneal | | | |
| dystrophy | | | |
| Galactosemia | Carbo- | Various- | GALT, GALK1, and GALE |
| | hydrate | where | |
| | metab- | galactose | |
| | olism | accu- | |
| | disorder | mulates- | |
| | | liver, | |
| | | brain, | |
| | | eyes | |
| Gastro- | | | CISH |
| intestinal | | | |
| Epithelial | | | |
| Cancer, GI | | | |
| cancer | | | |
| Gaucher | Fat | Various- | GBA |
| Disease | metab- | liver, | |
| (Types 1, 2, | olism | spleen, | |
| and 3, as | disorder | blood, | |
| well as | | CNS, | |
| other unusual | | skeletal | |
| forms that | | system | |
| may not fit | | | |
| into these | | | |
| types) | | | |
| Griscelli | | | |
| syndrome | | | |
| Glaucoma | eye | | MYOC, TIGR, GLC1A, |
| | | | JOAG, GPOA, OPTN, |
| | | | GLC1E, FIP2, HYPL, NRP, |
| | | | CYP1B1, GLC3A, OPA1, |
| | | | NTG, NPG, CYP1B1, |
| | | | GLC3A, those described in |
| | | | WO2015153780 |
| Glomerulo | kidney | | CC chemokine ligand 2 |
| sclerosis | | | |
| Glycogen | Metab- | | SLC2A2, GLUT2, |
| Storage | olism | | G6PC, G6PT, G6PT1, |
| Diseases | Diseases | | GAA, LAMP2, |
| Types I-VI- | | | LAMPB, AGL, GDE, |
| See also | | | GBE1, GYS2, PYGL, |
| Cori's | | | PFKM, see also |
| Disease, | | | Cori's Disease, |
| Pompe's | | | Pompe's Disease, |
| Disease, | | | McArdle's disease, |
| McArdle's | | | Hers Disease, and Von |
| disease, | | | Gierke's disease |
| Hers Disease, | | | |
| and Von | | | |
| Gierke's | | | |
| disease | | | |
| RBC | blood | | any mutations in a |
| Glycolytic | | | gene for an enzyme in |
| enzyme | | | the glycolysis pathway |
| deficiency | | | including mutations |
| | | | in genes for hexokinases |
| | | | I and II, glucokinase, |
| | | | phosphoglucose isomerase, |
| | | | phosphofructokinase, |
| | | | aldolase Bm |
| | | | triosephosphate isomerease, |
| | | | glyceraldehydee-3-phosphate |
| | | | dehydrogenase, |
| | | | phosphoglycerokinase, |
| | | | phosphoglycerate mutase, |
| | | | enolase I, |
| | | | pyruvate kinase |
| Hartnup's | Mal- | Various- | SLC6A19 |
| disease | absorption | brain, | |
| | disease | gastro- | |
| | | intestinal, | |
| | | skin, | |
| Hearing Loss | ear | | NOX3, Hes5, BDNF, |
| Hemo- | Iron | Various- | HFE and H63D |
| chromatosis | absorption | wherever | |
| (HH) | regulation | iron | |
| | disease | accu- | |
| | | mulates, | |
| | | liver, | |
| | | heart, | |
| | | pancreas | |
| | | joints, | |
| | | pituitary | |
| | | gland | |
| Hemo- | blood | | PRF1, HPLH2, |
| phagocytic | | | UNC13D, MUNC13-4, |
| lympho- | | | HPLH3, HLH3, FHL3 |
| histiocytosis | | | |
| disorders | | | |
| Hemorrhagic | blood | | PI, ATT, F5 |
| disorders | | | |
| Hers disease | liver | muscle | PYGL |
| (Glycogen | | | |
| storage | | | |
| disease | | | |
| Type VI) | | | |
| Hereditary | | | kalikrein B1 |
| angioedema | | | |
| (HAE) | | | |
| Hereditary | Skin and | | ACVRL1, ENG |
| Hemorrhagic | mucous | | and SMAD4 |
| Telangiectasia | mem- | | |
| (Osler- | branes | | |
| Weber-Rendu | | | |
| Syndrome) | | | |
| Hereditary | blood | | NK1, EPB42, SLC4A1, |
| Spherocytosis | | | SPTA1, and SPTB |
| Hereditary | blood | | HBG1, HBG2, BCL11A, |
| Persistence | | | promoter region |
| of Fetal | | | of HBG 1 and/or 2 |
| Hemoglobin | | | (in the CCAAT box) |
| Hemophilia | blood | | A: FVIII, F8C, HEMA |
| (hemophilia A | | | B: FVIX, HEMB |
| (Classic) | | | C: F9, F11 |
| a B (aka | | | |
| Christmas | | | |
| disease) and | | | |
| C) | | | |
| Hepatic | liver | | TCF1, HNF1A, MODY3 |
| adenoma | | | |
| Hepatic | liver | | SCOD1, SCO1 |
| failure, early | | | |
| onset, and | | | |
| neurologic | | | |
| disorder | | | |
| Hepatic lipase | liver | | LIPC |
| deficiency | | | |
| Hepato- | liver | | CTNNB1, PDGFRL, |
| blastoma, | | | PDGRL, PRLTS, |
| cancer and | | | AXIN1, AXIN, CTNNB1, |
| carcinomas | | | TP53, P53, LFS1, IGF2R, |
| | | | MPRI, MET, CASP8, |
| | | | MCH5 |
| Hermansky- | Skin, | | HPS1, HPS3, HPS4, |
| Pudlak | eyes, | | HPS5, HPS6, HPS7, |
| syndrome | blood, | | DTNBP1, BLOC1, |
| | lung, | | BLOC1S2, BLOC3 |
| | kidneys, | | |
| | intestine | | |
| HIV | Immune | | IL10, CSIF, CMKBR2, |
| susceptibility | system | | CCR2, CMKBR5, |
| or infection | | | CCCKR5 (CCR5), those in |
| | | | WO2015148670A1 |
| Holo- | brain | | ACVRL1, ENG, SMAD4 |
| prosencephaly | | | |
| (HPE) | | | |
| (Alobar, | | | |
| Semilobar, | | | |
| and Lobar) | | | |
| Homo- | Metabolic | Various- | CBS, MTHFR, |
| cystinuria | disease | connec- | MTR, MTRR, and |
| | | tive | MMADHC |
| | | tissue, | |
| | | muscles, | |
| | | CNS, | |
| | | cardio- | |
| | | vascular | |
| | | system | |
| HPV | | | HPV16 and HPV18 E6/E7 |
| HSV1, | eye | | HSV1 genes |
| HSV2, and | | | (immediate early and late |
| related | | | HSV-1 genes (UL1, 1.5, |
| | | | 5, 6, 8, 9, 12, 15, |
| keratitis | | | 16, 18, 19, 22, 23, 26, |
| | | | 26.5, 27, 28, 29, 30, |
| | | | 31, 32, 33, 34, 35, 36, |
| | | | 37, 38, 42, 48, 49.5, |
| | | | 50, 52, 54, S6, RL2, |
| | | | RS1, those described |
| | | | in WO2015153789, |
| | | | WO2015153791 |
| Hunter's | Lysosomal | Various- | IDS |
| Syndrome | storage | liver, | |
| (aka | disease | spleen, | |
| Mucopoly- | | eye, | |
| saccharidosis | | joint, | |
| type II) | | heart, | |
| | | brain, | |
| | | skeletal | |
| Huntington's | Brain, | | HD, HTT, IT15, |
| disease | nervous | | PRNP, PRIP, JPH3, JP3, |
| (HD) and | system | | HDL2, TBP, SCA17, |
| HD-like | | | PRKCE; IGF1; |
| disorders | | | EP300; RCOR1; PRKCZ; |
| | | | HDAC4; and TGM2, and |
| | | | those described in |
| | | | WO2013130824, |
| | | | WO2015089354 |
| Hurler's | Lysosomal | Various- | IDUA, α-L-iduronidase |
| Syndrome | storage | liver, | |
| (aka | disease | spleen, | |
| mucopoly- | | eye, | |
| saccharidosis | | joint, | |
| type I H, | | heart, | |
| MPS IH) | | brain, | |
| | | skeletal | |
| Hurler-Scheie | Lysosomal | Various- | IDUA, α-L-iduronidase |
| syndrome | storage | liver, | |
| (aka | disease | spleen, | |
| mucopoly- | | eye, | |
| saccharidosis | | joint, | |
| type I H-S, | | heart, | |
| MPS I H-S) | | brain, | |
| | | skeletal | |
| hyaluronidase | Soft and | | HYAL1 |
| deficiency | connective | | |
| (aka MPS IX) | tissues | | |
| Hyper IgM | Immune | | CD40L |
| syndrome | system | | |
| Hyper-tension | kidney | | Mineral corticoid receptor |
| caused | | | |
| renal damage | | | |
| Immuno- | Immune | | CD3E, CD3G, AICDA, |
| deficiencies | System | | AID, HIGM2, TNFRSF5, |
| | | | CD40, UNG, DGU, HIGM4, |
| | | | TNFSF5, CD40LG, |
| | | | HIGM1, IGM, FOXP3, |
| | | | IPEX, AIID, XPID, PIDX, |
| | | | TNFRSF14B, TACI |
| Inborn | Metab- | Various | See also: |
| errors of | olism | organs | Carbohydrate metabolism |
| metabolism: | diseases, | and cells | disorders (e.g. |
| including | liver | | galactosemia), Amino acid |
| urea cycle | | | Metabolism |
| disorders, | | | disorders (e.g. |
| organic | | | phenylketonuria), |
| acidemias), | | | Fatty acid metabolism |
| fatty acid | | | (e.g. MCAD deficiency), |
| oxidation | | | Urea Cycle disorders |
| defects, amino | | | (e.g. Citrullinemia), Organic |
| acidopathies, | | | acidemias (e.g. Maple |
| carbohydrate | | | Syrup Urine disease), |
| disorders, | | | Mitochondrial disorders (e.g. |
| mitochondrial | | | MELAS), peroxisomal |
| disorders | | | disorders (e.g. |
| | | | Zellweger syndrome) |
| Inflammation | Various | | IL-10; IL-1 (IL-1a; |
| | | | IL-1b); IL-13; IL-17 |
| | | | (IL-17a (CTLA8); IL- |
| | | | 17b; IL-17c; IL-17d; |
| | | | IL-17f); II-23; |
| | | | Cx3cr1; ptpn22; TNFa; |
| | | | NOD2/CARD15 for |
| | | | IBD; IL-6; IL-12 (IL- |
| | | | 12a; IL-12b); |
| | | | CTLA4; Cx3c11 |
| Inflammatory | Gastro- | Joints, | NOD2, IRGM, LRRK2, |
| Bowel | intestinal | skin | ATG5, ATG16L1, |
| Diseases (e.g. | | | IRGM, GATM, ECM1, |
| Ulcerative | | | CDH1, LAMB1, |
| Colitis and | | | HNF4A, GNA12, IL10, |
| Chron's | | | CARD9/15. CCR6, |
| Disease) | | | IL2RA, MST1, TNFSF15, |
| | | | REL, STAT3, |
| | | | IL23R, IL12B, FUT2 |
| Interstitial | kidney | | TGF-β type II receptor |
| renal | | | |
| fibrosis | | | |
| Job's | Immune | | STAT3, DOCK8 |
| Syndrome | System | | |
| (aka | | | |
| Hyper IgE | | | |
| Syndrome) | | | |
| Juvenile | eye | | RS1, XLRS1 |
| Retinoschisis | | | |
| Kabuki | | | MLL4, KMT2D |
| Syndrome 1 | | | |
| Kennedy | Muscles, | | SBMA/SMAX1/AR |
| Disease (aka | brain, | | |
| Spinobulbar | nervous | | |
| Muscular | system | | |
| Atrophy) | | | |
| Klinefelter | Various- | | Extra X |
| syndrome | partic- | | chromosome in males |
| | ularly | | |
| | those | | |
| | involved | | |
| | in | | |
| | develop- | | |
| | ment | | |
| | of male | | |
| | charac- | | |
| | teristics | | |
| Lafora | Brain, | | EMP2A and EMP2B |
| Disease | CNS | | |
| Leber | eye | | CRB1, RP12, |
| Congenital | | | CORD2, CRD, CRX, |
| Amaurosis | | | IMPDH1, OTX2, AIPL1, |
| | | | CABP4, CCT2, |
| | | | CEP290, CLUAP1, CRB1, |
| | | | CRX, DTHD1, GDF6, |
| | | | GUCY2D, IFT140, IQCB1, |
| | | | KCNJ13, LCA5, LRAT, |
| | | | NMNAT1, PRPH2, RD3, |
| | | | RDH12, RPE65, RP20, |
| | | | RPGRIP1, SPATA7, |
| | | | TULP1, LCA1, LCA4, |
| | | | GUC2D, CORD6, LCA3, |
| Lesch-Nyhan | Metab- | Various- | HPRT1 |
| Syndrome | olism | joints, | |
| | disease | cognitive, | |
| | | brain, | |
| | | nervous | |
| | | system | |
| Leukocyte | blood | | ITGB2, CD18, LCAMB, |
| deficiencies | | | LAD, EIF2B1, EIF2BA, |
| and disorders | | | EIF2B2, EIF2B3, EIF2B5, |
| | | | LVWM, CACH, |
| | | | CLE, EIF2B4 |
| Leukemia | Blood | | TAL1, TCL5, SCL, TAL2, |
| | | | FLT3, NBS1, NBS, |
| | | | ZNFN1A1, IK1, LYF1, |
| | | | HOXD4, HOX4B, |
| | | | BCR, CML, |
| | | | PHL, ALL, ARNT, KRAS2, |
| | | | RASK2, GMPS, AF10, |
| | | | ARHGEF12, LARG, |
| | | | KIAA0382, CALM, CLTH, |
| | | | CEBPA, CEBP, CHIC2, BTL, |
| | | | FLT3, KIT, PBT, LPP, NPM1, |
| | | | NUP214, D9S46E, CAN, |
| | | | CAIN, RUNX1, CBFA2, |
| | | | AML1, WHSC1L1, NSD3, |
| | | | FLT3, AF1Q, |
| | | | NPM1, NUMA1, ZNF145, |
| | | | PLZF, PML, MYL, STAT5B, |
| | | | AF10, CALM, CLTH, ARL11, |
| | | | ARLTS1, P2RX7, P2X7, BCR, |
| | | | CML, PHL, ALL, GRAF, |
| | | | NF1, VRNF, WSS, NFNS, |
| | | | PTPN11, PTP2C, SHP2, NS1, |
| | | | BCL2, CCND1, PRAD1, |
| | | | BCL1, TCRA, GATA1, GF1, |
| | | | ERYF1, NFE1, |
| | | | ABL1, NQO1, DIA4, NMOR1, |
| | | | NUP214, D9S46E, |
| | | | CAN, CAIN |
| Limb-girdle | muscle | | LGMD |
| muscular | | | |
| dystrophy | | | |
| diseases | | | |
| Lowe | brain, | | OCRL |
| syndrome | eyes, | | |
| | kidneys | | |
| Lupus | kidney | | MAPK1 |
| glomerulo- | | | |
| nephritis | | | |
| Machado- | Brain, | | ATX3 |
| Joseph's | CNS, | | |
| Disease (also | muscle | | |
| known as | | | |
| Spino- | | | |
| cerebellar | | | |
| ataxia | | | |
| Type 3) | | | |
| Macular | eye | | ABC4, CBC1, CHM1, |
| degeneration | | | APOE, C1QTNF5, C2, C3, |
| | | | CCL2, CCR2, CD36, |
| | | | CFB, CFH, |
| | | | CFHR1, CFHR3, CNGB3, |
| | | | CP, CRP, CST3, CTSD, |
| | | | CX3CR1, ELOVL4, ERCC6, |
| | | | FBLN5, FBLN6, FSCN2, |
| | | | HMCN1, HTRA1, IL6, IL8, |
| | | | PLEKHA1, PROM1, |
| | | | PRPH2, RPGR, SERPING1, |
| | | | TCOF1 ,TIMP3, TLR3 |
| Macular | eye | | BEST1, C1QTNF5, CTNNA1, |
| Dystrophy | | | EFEMP1, ELOVL4, |
| | | | FSCN2, GUCA1B, HMCN1, |
| | | | IMPG1, OTX2, |
| | | | PRDM13, PROM1, PRPH2, |
| | | | RP1L1, TIMP3, ABCA4, |
| | | | CFH, DRAM2, |
| | | | IMG1, MFSD8, |
| | | | ADMD, STGD2, STGD3, |
| | | | RDS, RP7, PRPH, AVMD, |
| | | | AOFMD, VMD2 |
| Malattia | eye | | EFEMP1, FBLN3 |
| Leventinesse | | | |
| Maple | Metab- | | BCKDHA, BCKDHB, |
| Syrup Urine | olism | | and DBT |
| Disease | disease | | |
| Marfan | Connec- | Musculo- | FBN1 |
| syndrome | tive | skeletal | |
| | tissue | | |
| Maroteaux- | Musculo- | Liver, | ARSB |
| Lamy | skeletal | spleen | |
| Syndrome | system, | | |
| (aka MPS | nervous | | |
| VI) | system | | |
| McArdle's | Glycogen | muscle | PYGM |
| Disease | storage | | |
| (Glycogen | disease | | |
| Storage | | | |
| Disease | | | |
| Type V) | | | |
| Medullary | kidney | | UMOD, HNFJ, |
| cystic | | | FJHN, MCKD2, |
| kidney disease | | | ADMCKD2 |
| Meta- | Lysosomal | Nervous | ARSA |
| chromatic | storage | system | |
| leuko- | disease | | |
| dystrophy | | | |
| Methyl- | Metab- | | MMAA, MMAB, |
| malonic | olism | | MUT, MMACHC, |
| acidemia | disease | | MMADHC, LMBRD1 |
| (MMA) | | | |
| Morquio | Connec- | heart | GALNS |
| Syndrome | tive | | |
| (aka MPS | tissue, | | |
| IV A and B) | skin, | | |
| | bone, eyes | | |
| Mucopoly- | Lysosomal | | See also Hurler/ |
| saccharidosis | storage | | Scheie syndrome, Hurler |
| diseases | disease- | | disease, Sanfillipo |
| (Types I H/S, | affects | | syndrome, Scheie |
| I H, II, III A | various | | syndrome, Morquio |
| B and C, I | organs/ | | syndrome, |
| S, IVA and | tissues | | hyaluronidase |
| B, IX, VII, | | | deficiency, Sly syndrome, |
| and VI) | | | and Maroteaux- |
| | | | Lamy syndrome |
| Muscular | muscle | | VAPB, VAPC, ALS8, SMN1, |
| Atrophy | | | SMA1, SMA2, SMA3, SMA4, |
| | | | BSCL2, SPG17, GARS, |
| | | | SMAD1, CMT2D, HEXB, |
| | | | IGHMBP2, SMUBP2, |
| | | | CATF1, SMARD1 |
| Muscular | muscle | | FKRP, MDC1C, |
| dystrophy | | | LGMD2I, LAMA2, |
| | | | LAMM, LARGE, KIAA0609, |
| | | | MDC1D, FCMD, TTID, |
| | | | MYOT, CAPN3, CANP3, |
| | | | DYSF, LGMD2B, SGCG, |
| | | | LGMD2C, DMDA1, |
| | | | SCG3, SGCA, ADL, DAG2, |
| | | | LGMD2D, DMDA2, SGCB, |
| | | | LGMD2E, SGCD, |
| | | | SGD, LGMD2F, |
| | | | CMD1L, TCAP, LGMD2G, |
| | | | CMD1N, TRIM32, HT2A, |
| | | | LGMD2H, FKRP, MDC1C, |
| | | | LGMD2I, TTN, |
| | | | CMD1G, TMD, |
| | | | LGMD2J, POMT1, CAV3, |
| | | | LGMD1C, SEPN1, SELN, |
| | | | RSMD1, PLEC1, PLTN, EBS1 |
| Myotonic | Muscles | Eyes, | CNBP (Type 2) |
| dystrophy | | heart, | and DMPK (Type 1) |
| (Type 1 | | endocrine | |
| and Type 2) | | | |
| Neoplasia | | | PTEN; ATM; ATR; EGFR; |
| | | | ERBB2; ERBB3; |
| | | | ERBB4; Notch1; Notch2; |
| | | | Notch3; Notch4; AKT; |
| | | | AKT2; AKT3; HIF; |
| | | | HIF1a; HIF3a; Met; HRG; |
| | | | Bc12; PPAR alpha; PPAR |
| | | | gamma; WT1 (Wilms Tumor); |
| | | | FGF Receptor Family |
| | | | members (5 members: |
| | | | 1, 2, 3, 4, 5); |
| | | | CDKN2a; APC; RB |
| | | | (retinoblastoma); MEN1; |
| | | | VHL; BRCA1; BRCA2; AR |
| | | | (Androgen |
| | | | Receptor); TSG101; |
| | | | IGF; IGF Receptor; Igf1 (4 |
| | | | variants); Igf2 (3 variants); |
| | | | Igf 1 Receptor; Igf 2 Receptor; |
| | | | Bax; Bc12; caspases family |
| | | | (9 members: 1, 2, 3, 4, |
| | | | 6, 7, 8, 9, 12); Kras; Apc |
| Neuro- | brain, | | NF1, NF2 |
| fibromatosis | spinal | | |
| (NF) (NF1, | cord, | | |
| formerly | nerves, | | |
| Reckling- | and skin | | |
| hausen's NF, | | | |
| and NF2) | | | |
| Niemann- | Lysosomal | Various- | Types A andB: |
| Pick | Storage | where | SMPD1; Type C: NPC1 or |
| Lipidosis | Disease | sphingo- | NPC2 |
| (Types A, B, | | myelin | |
| and C) | | accu- | |
| | | mulates, | |
| | | partic- | |
| | | ularly | |
| | | spleen, | |
| | | liver, | |
| | | blood, | |
| | | CNS | |
| Noonan | Various- | | PTPN11, SOS1, |
| Syndrome | musculo- | | RAF1 and KRAS |
| | skeletal, | | |
| | heart, | | |
| | eyes, | | |
| | repro- | | |
| | ductive | | |
| | organs, | | |
| | blood | | |
| Norrie | eye | | NDP |
| Disease or | | | |
| X-linked | | | |
| Familial | | | |
| Exudative | | | |
| Vitreo- | | | |
| retinopathy | | | |
| North | eye | | MCDRI |
| Carolina | | | |
| Macular | | | |
| Dystrophy | | | |
| Osteogenesis | bones, | | COL1A1, COL1A2, |
| imperfecta | musculo- | | CRTAP, P3H |
| (OI) (Types | skeletal | | |
| I, II, III, | | | |
| IV, V, VI, | | | |
| VII) | | | |
| Osteopetrosis | bones | | LRP5, BMND1, LRP7, |
| | | | LR3, OPPG, VBCH2, |
| | | | CLCN7, CLC7, OPTA2, |
| | | | OSTM1, GL, TCIRG1, |
| | | | TIRC7, OC116, OPTB1 |
| Patau's | Brain, | | Additional copy |
| Syndrome | heart, | | of chromosome 13 |
| (Trisomy 13) | skeletal | | |
| | system | | |
| Parkinson's | Brain, | | SNCA (PARK1), |
| disease | nervous | | UCHL1 (PARK 5), |
| (PD) | system | | and LRRK2 |
| | | | (PARK8), (PARK3), PARK2, |
| | | | PARK4, PARK7 |
| | | | (PARK7), PINK1 |
| | | | (PARK6); x-Synuclein, DJ-1, |
| | | | Parkin, NR4A2, NURR1, |
| | | | NOT, TINUR, SNCAIP, |
| | | | TBP, SCA17, NCAP, PRKN, |
| | | | PDJ, DBH, NDUFV2 |
| Pattern | eye | | RDS/peripherin |
| Dystrophy of | | | |
| the RPE | | | |
| Phenyl- | Metab- | Various | PAH, PKU1, |
| ketonuria | olism | due to | QDPR, DHPR, PTS |
| (PKU) | disorder | build- | |
| | | up of | |
| | | phenyl- | |
| | | alanine, | |
| | | phenyl | |
| | | ketones | |
| | | in tissues | |
| | | and CNS | |
| Polycystic | Kidney, | | FCYT, PKHD1, |
| kidney and | liver | | ARPKD, PKD1, PKD2, |
| hepatic | | | PKD4, PKDTS, PRKCSH, |
| disease | | | G19P1, PCLD, SEC63 |
| Pompe's | Glycogen | Various- | GAA |
| Disease | storage | heart, | |
| | disease | liver, | |
| | | spleen | |
| Porphyria | Various- | | ALAD, ALAS2, |
| (actually | wherever | | CPOX, FECH, HMBS, |
| refers to | heme | | PPOX, UROD, or UROS |
| a group of | precursors | | |
| different | accumulate | | |
| diseases all | | | |
| having a | | | |
| specific heme | | | |
| production | | | |
| process | | | |
| abnormality) | | | |
| posterior | eyes | | TCF4; COL8A2 |
| polymorphous | | | |
| corneal | | | |
| dystrophy | | | |
| Primary | Various- | | LDHA (lactate |
| Hyperoxaluria | eyes, | | dehydrogenase A) and |
| (e.g. type 1) | heart, | | hydroxyacid |
| | kidneys, | | oxidase 1 (HAO1) |
| | skeletal | | |
| | system | | |
| Primary | eyes | | MYOC |
| Open Angle | | | |
| Glaucoma | | | |
| (POAG) | | | |
| Primary | Liver, | | TCF4; COL8A2 |
| sclerosing | gall- | | |
| cholangitis | bladder | | |
| Progeria | All | | LMNA |
| (also called | | | |
| Hutchinson- | | | |
| Gilford | | | |
| progeria | | | |
| syndrome) | | | |
| Prader-Willi | Musculo- | | Deletion of region |
| Syndrome | skeletal | | of short arm of |
| | system, | | chromosome 15, |
| | brain, | | including UBE3A |
| | repro- | | |
| | ductive | | |
| | and | | |
| | endocrine | | |
| | system | | |
| Prostate | prostate | | HOXB13, MSMB, |
| Cancer | | | GPRC6A, TP53 |
| Pyruvate | Brain, | | PDHA1 |
| Dehydro- | nervous | | |
| genase | system | | |
| Deficiency | | | |
| Kidney/Renal | kidney | | RLIP76, VEGF |
| carcinoma | | | |
| Rett | Brain | | MECP2, RTT, PPMX, |
| Syndrome | | | MRX16, MRX79, CDKL5, |
| | | | STK9, MECP2, RTT, PPMX, |
| | | | MRX16, MRX79, |
| | | | x-Synuclein, DJ-1 |
| Retinitis | eye | | ADIPOR1, ABCA4, AGBL5, |
| pigmentosa | | | ARHGEF18, ARL2BP, |
| (RP) | | | ARL3, ARL6, BEST1, BBS1, |
| | | | BBS2, C2ORF71, C8ORF37, |
| | | | CA4, CERKL, CLRN1, |
| | | | CNGA1, CMGB1, CRB1, |
| | | | CRX, CYP4V2, DHDDS, |
| | | | DHX38, EMC1, |
| | | | EYS, FAM161A, |
| | | | FSCN2, GPR125, GUCA1B, |
| | | | HK1, HPRPF3, HGSNAT, |
| | | | IDH3B, IMPDH1, |
| | | | IMPG2, IFT140, |
| | | | IFT172, KLHL7, KIAA1549, |
| | | | KIZ, LRAT, MAK, MERTK, |
| | | | MVK, NEK2, |
| | | | NUROD1, NR2E3, |
| | | | NRL, OFD1, PDE6A, PDE6B, |
| | | | PDE6G, POMGNT1, PRCD, |
| | | | PROM1, PRPF3, |
| | | | PRPF4, PRPF6, |
| | | | PRPF8, PRPF31, PRPH2, |
| | | | RPB3, RDH12, REEP6, RP39, |
| | | | RGR, RHO, RLBP1, |
| | | | ROM1, RP1, RP1L1, |
| | | | RPY, RP2, RP9, RPE65, |
| | | | RPGR, SAMD11, |
| | | | SAG, SEMA4A, |
| | | | SLC7A14, SNRNP200, |
| | | | SPP2, SPATA7, TRNT1, |
| | | | TOPORS, TTC8, |
| | | | TULP1, USH2A, ZFN408, |
| | | | ZNF513, see also |
| | | | 20120204282 |
| Scheie | Various- | | IDUA, α-L-iduronidase |
| syndrome | liver, | | |
| (also | spleen, | | |
| known as | eye, | | |
| mucopoly- | joint, | | |
| saccharidosis | heart, | | |
| type I S | brain, | | |
| (MPS I-S)) | skeletal | | |
| Schizophrenia | Brain | | Neuregulin1 (Nrg1); Erb4 |
| | | | (receptor for Neuregulin); |
| | | | Complexin1 (Cplx1); Tph1 |
| | | | Tryptophan hydroxylase; Tph2 |
| | | | Tryptophan hydroxylase |
| | | | 2; Neurexin 1; |
| | | | GSK3; GSK3a; GSK3b; |
| | | | 5-HTT (Slc6a4); COMT; DRD |
| | | | (Drd1a); SLC6A3; |
| | | | DAOA; DTNBP1; |
| | | | Dao (Dao1); TCF4; COL8A2 |
| Secretase | Various | | APH-1 (alpha and beta); |
| Related | | | PSEN1; NCSTN; PEN-2; |
| Disorders | | | Nos1, Parp1, Nat1, Nat2, CTSB, |
| | | | APP, APH1B, PSEN2, |
| | | | PSENEN, BACE1, ITM2B, |
| | | | CTSD, NOTCH1, TNF, |
| | | | INS, DYT10, ADAM17, |
| | | | APOE, ACE, STN, TP53, |
| | | | IL6, NGFR, IL1B, ACHE, |
| | | | CTNNB1, IGF1, IFNG, |
| | | | NRG1, CASP3, MAPK1, |
| | | | CDH1, APBB1, |
| | | | HMGCR, CREB1, |
| | | | PTGS2, HES1, CAT, TGFB1, |
| | | | ENO2, ERBB4, TRAPPC10, |
| | | | MAOB, NGF, MMP12, JAG1, |
| | | | CD40LG, PPARG, FGF2, |
| | | | LRP1, NOTCH4, |
| | | | MAPK8, PREP, |
| | | | NOTCH3, PRNP, CTSG, |
| | | | EGF, REN, CD44, SELP, |
| | | | GHR, ADCYAP1, INSR, |
| | | | GFAP, MMP3, |
| | | | MAPK10, SP1, |
| | | | MYC, CTSE, PPARA, JUN, |
| | | | TIMP1, IL5, IL1A, MMP9, |
| | | | HTR4, HSPG2, |
| | | | KRAS, CYCS, |
| | | | SMG1, IL1R1, PROK1, |
| | | | MAPK3, NTRK1, |
| | | | IL13, MME, |
| | | | TKT, CXCR2, CHRM1, |
| | | | ATXN1, PAWR, |
| | | | NOTCJ2, M6PR, CYP46A1, |
| | | | CSNK1D, MAPK14, |
| | | | PRG2, PRKCA, L1 CAM, |
| | | | CD40, NR1I2, JAG2, |
| | | | CTNND1, CMA1, SORT1, |
| | | | DLK1, THEM4, JUP, CD46, |
| | | | CCL11, CAV3, |
| | | | RNASE3, HSPA8, CASP9, |
| | | | CYP3A4, CCR3, TFAP2A, |
| | | | SCP2, CDK4, |
| | | | JOF1A, TCF7L2, |
| | | | B3GALTL, MDM2, RELA, |
| | | | CASP7, IDE, FANP4, CASK, |
| | | | ADCYAP1R1, |
| | | | ATF4, PDGFA, |
| | | | C21ORF33, SCG5, RMF123, |
| | | | NKFB1, ERBB2, |
| | | | CAV1, MMP7, TGFA, |
| | | | RXRA, STX1A, PSMC4, |
| | | | P2RY2, TNFRSF21, DLG1, |
| | | | NUMBL, SPN, PLSCR1, |
| | | | UBQLN2, UBQLN1, PCSK7, |
| | | | SPON1, SILV, QPCT, |
| | | | HESS, GCC1 |
| Selective IgA | Immune | | Type 1: MSH5; Type 2: |
| Deficiency | system | | TNFRSF13B |
| Severe | Immune | | JAK3, JAKL, DCLRE1C, |
| Combined | system | | ARTEMIS, |
| Immuno- | | | SCIDA, RAG1, RAG2, |
| deficiency | | | ADA, PTPRC, CD45, LCA, |
| (SCID) and | | | IL7R, CD3D, T3D, IL2RG, |
| SCID-X1, | | | SCIDX1, SCIDX, IMD4, |
| and ADA- | | | those identified in |
| SCID | | | US Pat. App. |
| | | | Pub. 20110225664, |
| | | | 20110091441, 20100229252, |
| | | | 20090271881 |
| | | | and 20090222937; |
| Sickle cell | blood | | HBB, BCL11A, |
| disease | | | BCLUAe, cis-regulatory |
| | | | elements of the |
| | | | B-globin locus, HBG 1/2 |
| | | | promoter, HBG distal |
| | | | CCAAT box region |
| | | | between −92 and |
| | | | −130 of the HBG |
| | | | Transcription Start Site, |
| | | | those described in |
| | | | WO2015148863, |
| | | | WO 2013/126794, US |
| | | | Pat Pub. 20110182867 |
| Sly Syndrome | | | GUSB |
| (aka MPS | | | |
| VII) | | | |
| Spino- | | | ATXN1, ATXN2, ATX3 |
| cerebellar | | | |
| Ataxias (SCA | | | |
| types 1, | | | |
| 2, 3, 6, 7, | | | |
| 8, 12 and 17) | | | |
| Sorsby | eye | | TIMP3 |
| Fundus | | | |
| Dystrophy | | | |
| Stargardt | eye | | ABCR, ELOVL4, |
| disease | | | ABCA4, PROM1 |
| Tay-Sachs | Lysosomal | Various- | HEX-A |
| Disease | Storage | CNS, | |
| | disease | brain, | |
| | | eye | |
| Thalassemia | blood | | HBA1, HBA2 (Alpha), |
| (Alpha, | | | HBB (Beta), HBB |
| Beta, Delta) | | | and HBD (delta), |
| | | | LCRB, BCL11A, |
| | | | BCL11Ae, cis-regulatory |
| | | | elements of the |
| | | | B-globin locus, HBG |
| | | | 1/2 promoter, those |
| | | | described in |
| | | | WO2015148860, |
| | | | US Pat. Pub. |
| | | | 20110182867, 2015/148860 |
| Thymic | Immune | | deletion of 30 to 40 |
| Aplasia | system, | | genes in the middle |
| (DiGeorge | thymus | | of chromosome 22 |
| Syndrome; | | | at a location known |
| 22q11.2 | | | as 22q11.2, including |
| deletion | | | TBX1, DGCR8 |
| syndrome) | | | |
| Transthyretin | liver | | TTR (transthyretin) |
| amyloidosis | | | |
| (ATTR) | | | |
| trimethyl- | Metab- | | FMO3 |
| aminuria | olism | | |
| | disease | | |
| Trinucleotide | Various | | HTT; SBMA/ |
| Repeat | | | SMAX1/AR; |
| Disorders | | | FXN/X25 ATX3; |
| (generally) | | | ATXN1; ATXN2; |
| | | | DMPK; Atrophin-1 and Ata1 |
| | | | (DRPLA Dx); CBP |
| | | | (Creb-BP-global |
| | | | instability); VLDLR; |
| | | | Atxn7; Atxn10; |
| | | | FEN1, TNRC6A, |
| | | | PABPN1, JPH3, MED15, |
| | | | ATXN1, ATXN3, |
| | | | TBP, CACNA1A, |
| | | | ATXN80S, PPP2R2B, |
| | | | ATXN7, TNRC6B, |
| | | | TNRC6C, CELF3, |
| | | | MAB21L1, MSH2, |
| | | | TMEM185A, SIX5, |
| | | | CNPY3, RAXE, |
| | | | GNB2, RPL14, ATXN8, |
| | | | ISR, TTR, EP400, GIGYF2, |
| | | | OGG1, STC1, CNDP1, |
| | | | C10ORF2, MAML3, |
| | | | DKC1, PAXIP1, |
| | | | CASK, MAPT, SP1, |
| | | | POLG, AFF2, |
| | | | THBS1, TP53, ESR1, |
| | | | CGGBP1, ABT1, |
| | | | KLK3, PRNP, JUN, |
| | | | KCNN3, BAX, |
| | | | FRAXA, KBTBD10, |
| | | | MBNL1, RAD51, |
| | | | NCOA3, ERDA1, TSC1, |
| | | | COMP, GGLC, |
| | | | RRAD, MSH3, |
| | | | DRD2, CD44, CTCF, |
| | | | CCND1, CLSPN, |
| | | | MEF2A, PTPRU, |
| | | | GAPDH, TRIM22, |
| | | | WT1, AHR, GPX1, |
| | | | TPMT, NDP, ARX, |
| | | | TYR, EGR1, UNG, |
| | | | NUMBL, FABP2, |
| | | | EN2, CRYGC, SRP14, |
| | | | CRYGB, PDCD1, |
| | | | HOXA1, ATXN2L, |
| | | | PMS2, GLA, CBL, |
| | | | FTH1, IL12RB2, |
| | | | OTX2, HOXA5, |
| | | | POLG2, DLX2, |
| | | | AHRR, MANF, RMEM158, |
| | | | see also 20110016540 |
| Turner's | Various- | | Monosomy X |
| Syndrome | repro- | | |
| (XO) | ductive | | |
| | organs, | | |
| | and sex | | |
| | charac- | | |
| | teristics, | | |
| | vas- | | |
| | culature | | |
| Tuberous | CNS, | | TSC1, TSC2 |
| Sclerosis | heart, | | |
| | kidneys | | |
| Usher | Ears, eyes | | ABHD12, CDH23, |
| syndrome | | | CIB2, CLRN1, DFNB31, |
| (Types I, II, | | | GPR98, HARS, MYO7A, |
| and III) | | | PCDH15, USH1C, |
| | | | USH1G, USH2A, |
| | | | USH11A, those described in |
| | | | WO2015134812A1 |
| Velo- | Various- | | Many genes are deleted, |
| cardiofacial | skeletal, | | COM, TBX1, and |
| syndrome | heart, | | other are associated |
| (aka 22q11.2 | kidney, | | with symptoms |
| deletion | immune | | |
| syndrome, | system, | | |
| DiGeorge | brain | | |
| syndrome, | | | |
| conotruncal | | | |
| anomaly | | | |
| face | | | |
| syndrome | | | |
| (CTAF), | | | |
| autosomal | | | |
| dominant | | | |
| Opitz G/BB | | | |
| syndrome | | | |
| or Cayler | | | |
| cardiofacial | | | |
| syndrome) | | | |
| Von Gierke's | Glycogen | Various- | G6PC and SLC37A4 |
| Disease | Storage | liver, | |
| (Glycogen | disease | kidney | |
| Storage | | | |
| Disease | | | |
| type I) | | | |
| Von Hippel- | Various- | CNS, | VHL |
| Lindau | cell | Kidney, | |
| Syndrome | growth | Eye, | |
| | regulation | visceral | |
| | disorder | organs | |
| Von | blood | | VWF |
| Willebrand | | | |
| Disease | | | |
| (Types I, II | | | |
| and III) | | | |
| Wilson | Various- | Liver, | ATP7B |
| Disease | Copper | brains, | |
| | Storage | eyes, | |
| | Disease | other | |
| | | tissues | |
| | | where | |
| | | copper | |
| | | builds up | |
| Wiskott- | Immune | | WAS |
| Aldrich | System | | |
| Syndrome | | | |
| Xeroderma | Skin | Nervous | POLH |
| Pigmentosum | | system | |
| XXX | Endocrine, | | X chromosome |
| Syndrome | brain | | trisomy |

In some embodiments, the compositions, systems, or components thereof can be used treat or prevent a disease in a subject by modifying one or more genes associated with one or more cellular functions, such as any one or more of those in Table 9. In some embodiments, the disease is a genetic disease or disorder. In some of embodiments, the composition, system, or component thereof can modify one or more genes or polynucleotides associated with one or more genetic diseases such as any set forth in Table 9.

TABLE 9
Exemplary Genes controlling Cellular Functions

| CELLULAR | |
|---|---|
| FUNCTION | GENES |
| PI3K/AKT | PRKCE; ITGAM; ITGA5; IRAK1; |
| Signaling | PRKAA2; EIF2AK2; PTEN; EIF4E; |
| | PRKCZ; GRK6; MAPK1; TSC1; |
| | PLK1; AKT2; IKBKB; PIK3CA; CDK8; |
| | CDKN1B; NFKB2; BCL2; PIK3CB; |
| | PPP2R1A; MAPK8; BCL2L1; MAPK3; |
| | TSC2; ITGA1; KRAS; EIF4EBP1; |
| | RELA; PRKCD; NOS3; |
| | PRKAA1; MAPK9; CDK2; |
| | PPP2CA; PIM1; ITGB7; |
| | YWHAZ; ILK; TP53; RAF1; |
| | IKBKG; RELB; DYRK1A; |
| | CDKN1A; ITGB1; MAP2K2; |
| | JAK1; AKT1; JAK2; PIK3R1; |
| | CHUK; PDPK1; PPP2R5C; |
| | CTNNB1; MAP2K1; NFKB1; |
| | PAK3; ITGB3; CCND1; GSK3A; |
| | FRAP1; SFN; ITGA2; |
| | TTK; CSNK1A1; BRAF; |
| | GSK3B; AKT3; FOXO1; SGK; |
| | HSP90AA1; RPS6KB1 |
| ERK/MAPK | PRKCE; ITGAM; ITGA5; |
| Signaling | HSPB1; IRAK1; PRKAA2; |
| | EIF2AK2; RAC1; RAP1A; |
| | TLN1; EIF4E; ELK1; GRK6; |
| | MAPK1; RAC2; PLK1; AKT2; |
| | PIK3CA; CDK8; CREB1; |
| | PRKCI; PTK2; FOS; |
| | RPS6KA4; PIK3CB; PPP2R1A; |
| | PIK3C3; MAPK8; MAPK3; ITGA1; |
| | ETS1; KRAS; MYCN; |
| | EIF4EBP1; PPARG; PRKCD; |
| | PRKAA1; MAPK9; SRC; |
| | CDK2; PPP2CA; PIM1; |
| | PIK3C2A; ITGB7; YWHAZ; |
| | PPP1CC; KSR1; PXN; |
| | RAF1; FYN; DYRK1A; ITGB1; |
| | MAP2K2; PAK4; PIK3R1; |
| | STAT3; PPP2R5C; MAP2K1; |
| | PAK3; ITGB3; ESR1; |
| | ITGA2; MYC; TTK; CSNK1A1; |
| | CRKL; BRAF; ATF4; PRKCA; |
| | SRF; STAT1; SGK |
| Gluco- | RAC1; TAF4B; EP300; |
| corticoid | SMAD2; TRAF6; PCAF; ELK1; |
| Receptor | MAPK1; SMAD3; AKT2; |
| Signaling | IKBKB; NCOR2; UBE2I; |
| | PIK3CA; CREB1; FOS; |
| | HSPA5; NFKB2; BCL2; |
| | MAP3K14; STAT5B; PIK3CB; |
| | PIK3C3; MAPK8; BCL2L1; |
| | MAPK3; TSC22D3; MAPK10; |
| | NRIP1; KRAS; MAPK13; |
| | RELA; STAT5A; MAPK9; |
| | NOS2A; PBX1; NR3C1; |
| | PIK3C2A; CDKN1C; TRAF2; |
| | SERPINE1; NCOA3; |
| | MAPK14; TNF; RAF1; |
| | IKBKG; MAP3K7; CREBBP; |
| | CDKN1A; MAP2K2; JAK1; IL8; |
| | NCOA2; AKT1; JAK2; |
| | PIK3R1; CHUK; STAT3; |
| | MAP2K1; NFKB1; TGFBR1; |
| | ESR1; SMAD4; CEBPB; JUN; |
| | AR; AKT3; CCL2; MMP1; |
| | STAT1; IL6; HSP90AA1 |
| Axonal | PRKCE; ITGAM; ROCK1; |
| Guidance | ITGA5; CXCR4; ADAM12; |
| Signaling | IGF1; RAC1; RAP1A; EIF4E; |
| | PRKCZ; NRP1; NTRK2; ARHGEF7; |
| | SMO; ROCK2; MAPK1; PGF; RAC2; |
| | PTPN11; GNAS; AKT2; |
| | PIK3CA; ERBB2; PRKCI; PTK2; |
| | CFL1; GNAQ; PIK3CB; |
| | CXCL12; PIK3C3; WNT11; |
| | PRKD1; GNB2L1; ABL1; |
| | MAPK3; ITGA1; KRAS; RHOA; |
| | PRKCD; PIK3C2A; ITGB7; |
| | GLI2; PXN; VASP; RAF1; |
| | FYN; ITGB1; MAP2K2; |
| | PAK4; ADAM17; AKT1; PIK3R1; |
| | GLI1; WNT5A; ADAM10; |
| | MAP2K1; PAK3; ITGB3; |
| | CDC42; VEGFA; ITGA2; |
| | EPHA8; CRKL; RND1; GSK3B; |
| | AKT3; PRKCA |
| Ephrin | PRKCE; ITGAM; ROCK1; |
| Receptor | ITGA5; CXCR4; IRAK1; |
| Signaling | PRKAA2; EIF2AK2; RAC1; |
| Actin | RAP1A; GRK6; ROCK2; |
| Cyto- | MAPK1; PGF; RAC2; PTPN11; |
| skeleton | GNAS; PLK1; AKT2; |
| Signaling | DOK1; CDK8; CREB1; |
| | PTK2; CFL1; GNAQ; MAP3K14; |
| | CXCL12; MAPK8; GNB2L1; |
| | ABL1; MAPK3; ITGA1; |
| | KRAS; RHOA; PRKCD; |
| | PRKAA1; MAPK9; SRC; CDK2; |
| | PIM1; ITGB7; PXN; RAF1; |
| | FYN; DYRK1A; ITGB1; |
| | MAP2K2; PAK4; AKT1; |
| | JAK2; STAT3; ADAM10; |
| | MAP2K1; PAK3; ITGB3; |
| | CDC42; VEGFA; ITGA2; |
| | EPHA8; TTK; CSNK1A1; |
| | CRKL; BRAF; PTPN13; ATF4; |
| | AKT3; SGK |
| | ACTN4; PRKCE; ITGAM; |
| | ROCK1; ITGA5; IRAK1; |
| | PRKAA2; EIF2AK2; RAC1; |
| | INS; ARHGEF7; GRK6; |
| | ROCK2; MAPK1; RAC2; |
| | PLK1; AKT2; PIK3CA; CDK8; |
| | PTK2; CFL1; PIK3CB; MYH9; |
| | DIAPH1; PIK3C3; MAPK8; |
| | F2R; MAPK3; SLC9A1; |
| | ITGA1; KRAS; RHOA; PRKCD; |
| | PRKAA1; MAPK9; CDK2; |
| | PIM1; PIK3C2A; ITGB7; |
| | PPP1CC; PXN; VIL2; |
| | RAF1; GSN; DYRK1A; ITGB1; |
| | MAP2K2; PAK4; PIP5K1A; |
| | PIK3R1; MAP2K1; PAK3; |
| | ITGB3; CDC42; APC; |
| | ITGA2; TTK; CSNK1A1; CRKL; |
| | BRAF; VAV3; SGK |
| Huntington's | PRKCE; IGF1; EP300; RCOR1; |
| Disease | PRKCZ; HDAC4; TGM2; |
| Signaling | MAPK1; CAPNS1; AKT2; |
| | EGFR; NCOR2; SP1; CAPN2; |
| | PIK3CA; HDAC5; CREB1; |
| | PRKCI; HSPA5; REST; |
| | GNAQ; PIK3CB; PIK3C3; |
| | MAPK8; IGF1R; PRKD1; |
| | GNB2L1; BCL2L1; CAPN1; |
| | MAPK3; CASP8; HDAC2; |
| | HDAC7A; PRKCD; HDAC11; |
| | MAPK9; HDAC9; PIK3C2A; |
| | HDAC3; TP53; CASP9; |
| | CREBBP; AKT1; PIK3R1; |
| | PDPK1; CASP1; APAF1; |
| | FRAP1; CASP2; JUN; BAX; |
| | ATF4; AKT3; PRKCA; |
| | CLTC; SGK; HDAC6; CASP3 |
| Apoptosis | PRKCE; ROCK1; BID; IRAK1; |
| Signaling | PRKAA2; EIF2AK2; BAK1; |
| | BIRC4; GRK6; MAPK1; |
| | CAPNS1; PLK1; AKT2; IKBKB; |
| | CAPN2; CDK8; FAS; NFKB2; |
| | BCL2; MAP3K14; MAPK8; |
| | BCL2L1; CAPN1; MAPK3; |
| | CASP8; KRAS; RELA; |
| | PRKCD; PRKAA1; MAPK9; |
| | CDK2; PIM1; TP53; TNF; |
| | RAF1; IKBKG; RELB; CASP9; |
| | DYRK1A; MAP2K2; |
| | CHUK; APAF1; MAP2K1; |
| | NFKB1; PAK3; LMNA; CASP2; |
| | BIRC2; TTK; CSNK1A1; BRAF; |
| | BAX; PRKCA; SGK; |
| | CASP3; BIRC3; PARP1 |
| B Cell | RAC1; PTEN; LYN; ELK1; |
| Receptor | MAPK1; RAC2; PTPN11; |
| Signaling | AKT2; IKBKB; PIK3CA; |
| | CREB1; SYK; NFKB2; CAMK2A; |
| | MAP3K14; PIK3CB; PIK3C3; |
| | MAPK8; BCL2L1; ABL1; |
| | MAPK3; ETS1; KRAS; |
| | MAPK13; RELA; PTPN6; MAPK9; |
| | EGR1; PIK3C2A; BTK; MAPK14; |
| | RAF1; IKBKG; RELB; |
| | MAP3K7; MAP2K2; AKT1; |
| | PIK3R1; CHUK; MAP2K1; |
| | NFKB1; CDC42; GSK3A; FRAP1; |
| | BCL6; BCL10; JUN; GSK3B; |
| | ATF4; AKT3; VAV3; RPS6KB1 |
| Leukocyte | ACTN4; CD44; PRKCE; |
| Extra- | ITGAM; ROCK1; CXCR4; CYBA; |
| vasation | RAC1; RAP1A; PRKCZ; |
| Signaling | ROCK2; RAC2; PTPN11; |
| | MMP14; PIK3CA; PRKCI; |
| | PTK2; PIK3CB; CXCL12; |
| | PIK3C3; MAPK8; PRKD1; |
| | ABL1; MAPK10; CYBB; |
| | MAPK13; RHOA; PRKCD; |
| | MAPK9; SRC; PIK3C2A; BTK; |
| | MAPK14; NOX1; PXN; VIL2; |
| | VASP; ITGB1; MAP2K2; |
| | CTNND1; PIK3R1; CTNNB1; |
| | CLDN1; CDC42; F11R; ITK; |
| | CRKL; VAV3; CTTN; |
| | PRKCA; MMP1; MMP9 |
| Integrin | ACTN4; ITGAM; ROCK1; |
| Signaling | ITGA5; RAC1; PTEN; RAP1A; |
| | TLN1; ARHGEF7; MAPK1; |
| | RAC2; CAPNS1; AKT2; |
| | CAPN2; PIK3CA; PTK2; |
| | PIK3CB; PIK3C3; MAPK8; |
| | CAV1; CAPN1; ABL1; |
| | MAPK3; ITGA1; KRAS; RHOA; |
| | SRC; PIK3C2A; ITGB7; |
| | PPP1CC; ILK; PXN; VASP; |
| | RAF1; FYN; ITGB1; |
| | MAP2K2; PAK4; AKT1; PIK3R1; |
| | TNK2; MAP2K1; PAK3; ITGB3; |
| | CDC42; RND3; ITGA2; |
| | CRKL; BRAF; GSK3B; AKT3 |
| Acute Phase | IRAK1; SOD2; MYD88; |
| Response | TRAF6; ELK1; MAPK1; PTPN11; |
| Signaling | AKT2; IKBKB; PIK3CA; |
| | FOS; NFKB2; MAP3K14; |
| | PIK3CB; MAPK8; RIPK1; |
| | MAPK3; IL6ST; KRAS; |
| | MAPK13; IL6R; RELA; |
| | SOCS1; MAPK9; FTL; NR3C1; |
| | TRAF2; SERPINE1; MAPK14; |
| | TNF; RAF1; PDK1; |
| | IKBKG; RELB; MAP3K7; |
| | MAP2K2; AKT1; JAK2; PIK3R1; |
| | CHUK; STAT3; MAP2K1; |
| | NFKB1; FRAP1; CEBPB; JUN; |
| | AKT3; IL1R1; IL6 |
| PTEN | ITGAM; ITGA5; RAC1; |
| Signaling | PTEN; PRKCZ; BCL2L11; |
| | MAPK1; RAC2; AKT2; |
| | EGFR; IKBKB; CBL; PIK3CA; |
| | CDKN1B; PTK2; NFKB2; |
| | BCL2; PIK3CB; BCL2L1; |
| | MAPK3; ITGA1; KRAS; |
| | ITGB7; ILK; PDGFRB; INSR; |
| | RAF1; IKBKG; CASP9; |
| | CDKN1A; ITGB1; MAP2K2; |
| | AKT1; PIK3R1; CHUK; |
| | PDGFRA; PDPK1; MAP2K1; |
| | NFKB1; ITGB3; CDC42; |
| | CCND1; GSK3A; ITGA2; |
| | GSK3B; AKT3; FOXO1; |
| | CASP3; RPS6KB1 |
| p53 | PTEN; EP300; BBC3; PCAF; |
| Signaling | FASN; BRCA1; GADD45A; |
| Aryl | BIRC5; AKT2; PIK3CA; |
| Hydro- | CHEK1; TP53INP1; BCL2; |
| carbon | PIK3CB; PIK3C3; MAPK8; |
| Receptor | THBS1; ATR; BCL2L1; E2F1; |
| Signaling | PMAIP1; CHEK2; TNFRSF10B; |
| | TP73; RB1; HDAC9; |
| | CDK2; PIK3C2A; MAPK14; |
| | TP53; LRDD; CDKN1A; |
| | HIPK2; AKT1; PIK3R1; |
| | RRM2B; APAF1; CTNNB1; |
| | SIRT1; CCND1; PRKDC; ATM; SFN; CDKN2A; JUN; |
| | SNAI2; GSK3B; BAX; AKT3 |
| | HSPB1; EP300; FASN; TGM2; RXRA; MAPK1; NQO1; |
| | NCOR2; SP1; ARNT; CDKN1B; FOS; CHEK1; |
| | SMARCA4; NFKB2; MAPK8; ALDH1A1; ATR; E2F1; |
| | MAPK3; NRIP1; CHEK2; RELA; TP73; GSTP1; RB1; |
| | SRC; CDK2; AHR; NFE2L2; NCOA3; TP53; TNF; |
| | CDKN1A; NCOA2; APAF1; |
| | NFKB1; CCND1; ATM; ESR1; |
| | CDKN2A; MYC; JUN; ESR2; BAX; IL6; CYP1B1; |
| | HSP90AA1 |
| Xenobiotic | PRKCE; EP300; PRKCZ; RXRA; MAPK1; NQO1; |
| Metabolism | NCOR2; PIK3CA; ARNT; PRKCI; NFKB2; CAMK2A; |
| Signaling | PIK3CB; PPP2R1A; PIK3C3; MAPK8; PRKD1; |
| | ALDH1A1; MAPK3; NRIP1; KRAS; MAPK13; |
| | PRKCD; GSTP1; MAPK9; NOS2A; |
| | ABCB1; AHR; PPP2CA; FTL; NFE2L2; |
| | PIK3C2A; PPARGC1A; MAPK14; TNF; RAF1; |
| | CREBBP; MAP2K2; PIK3R1; PPP2R5C; MAP2K1; |
| | NFKB1; KEAP1; PRKCA; EIF2AK3; IL6; CYP1B1; |
| | HSP90AA1 |
| SAPK/JNK | PRKCE; IRAK1; PRKAA2; EIF2AK2; RAC1; ELK1; |
| Signaling | GRK6; MAPK1; GADD45A; |
| | RAC2; PLK1; AKT2; PIK3CA; |
| | FADD; CDK8; PIK3CB; PIK3C3; MAPK8; RIPK1; |
| | GNB2L1; IRS1; MAPK3; MAPK10; DAXX; KRAS; |
| | PRKCD; PRKAA1; MAPK9; CDK2; PIM1; PIK3C2A; |
| | TRAF2; TP53; LCK; MAP3K7; DYRK1A; MAP2K2; |
| | PIK3R1; MAP2K1; PAK3; CDC42; |
| | JUN; TTK; CSNK1A1; |
| | CRKL; BRAF; SGK |
| PPAr/RXR | PRKAA2; EP300; INS; SMAD2; |
| Signaling | TRAF6; PPARA; FASN; |
| | RXRA; MAPK1; SMAD3; GNAS; IKBKB; NCOR2; |
| | ABCA1; GNAQ; NFKB2; MAP3K14; STAT5B; |
| | MAPK8; IRS1; MAPK3; KRAS; RELA; PRKAA1; |
| | PPARGC1A; NCOA3; MAPK14; INSR; |
| | RAF1; IKBKG; RELB; MAP3K7; |
| | CREBBP; MAP2K2; JAK2; CHUK; MAP2K1; NFKB1; |
| | TGFBR1; SMAD4; JUN; IL1R1; PRKCA; |
| | IL6; HSP90AA1; ADIPOQ |
| NF-KB | IRAK1; EIF2AK2; EP300; INS; |
| Signaling | MYD88; PRKCZ; TRAF6; TBK1; AKT2; |
| | EGFR; IKBKB; PIK3CA; BTRC; NFKB2; MAP3K14; |
| | PIK3CB; PIK3C3; MAPK8; RIPK1; HDAC2; |
| | KRAS; RELA; PIK3C2A; TRAF2; TLR4; PDGFRB; |
| | TNF; INSR; LCK; IKBKG; RELB; MAP3K7; |
| | CREBBP; AKT1; PIK3R1; CHUK; PDGFRA; NFKB1; |
| | TLR2; BCL10; GSK3B; AKT3; TNFAIP3; ILIR1 |
| Neuregulin | ERBB4; PRKCE; ITGAM; ITGA5; |
| Signaling | PTEN; PRKCZ; ELK1; |
| Wnt & | MAPK1; PTPN11; AKT2; EGFR; ERBB2; PRKCI; |
| Beta catenin | CDKN1B; STAT5B; PRKD1; MAPK3; ITGA1; KRAS; |
| Signaling | PRKCD; STAT5A; SRC; ITGB7; |
| | RAF1; ITGB1; MAP2K2; |
| | ADAM17; AKT1; PIK3R1; PDPK1; MAP2K1; ITGB3; |
| | EREG; FRAP1; PSEN1; ITGA2; MYC; NRG1; CRKL; |
| | AKT3; PRKCA; HSP90AA1; RPS6KB1 |
| | CD44; EP300; LRP6; DVL3; CSNK1E; GJA1; SMO; |
| | AKT2; PIN1; CDH1; BTRC; |
| | GNAQ; MARK2; PPP2R1A; |
| | WNT11; SRC; DKK1; PPP2CA; SOX6; SFRP2; ILK; |
| | LEF1; SOX9; TP53; MAP3K7; CREBBP; TCF7L2; |
| | AKT1; PPP2R5C; WNT5A; LRP5; CTNNB1; TGFBR1; |
| | CCND1; GSK3A; DVL1; APC; CDKN2A; MYC; |
| | CSNK1A1; GSK3B; AKT3; SOX2 |
| Insulin | PTEN; INS; EIF4E; PTPN1; PRKCZ; MAPK1; TSC1; |
| Receptor | PTPN11; AKT2; CBL; PIK3CA; |
| Signaling | PRKCI; PIK3CB; PIK3C3; |
| | MAPK8; IRS1; MAPK3; TSC2; KRAS; EIF4EBP1; |
| | SLC2A4; PIK3C2A; PPP1CC; INSR; RAF1; FYN; |
| | MAP2K2; JAK1; AKT1; JAK2; |
| | PIK3R1; PDPK1; MAP2K1; |
| | GSK3A; FRAP1; CRKL; GSK3B; AKT3; FOXO1; SGK; |
| | RPS6KB1 |
| IL-6 | HSPB1; TRAF6; MAPKAPK2; ELK1; MAPK1; |
| Signaling | PTPN11; IKBKB; FOS; NFKB2; MAP3K14; MAPK8; |
| | MAPK3; MAPK10; IL6ST; KRAS; MAPK13; |
| | IL6R; RELA; SOCS1; MAPK9; ABCB1; |
| | TRAF2; MAPK14; TNF; RAF1; IKBKG; RELB; |
| | MAP3K7; MAP2K2; IL8; JAK2; CHUK; STAT3; |
| | MAP2K1; NFKB1; CEBPB; JUN; ILIR1; SRF; IL6 |
| Hepatic | PRKCE; IRAK1; INS; MYD88; |
| Cholestasis | PRKCZ; TRAF6; PPARA; |
| | RXRA; IKBKB; PRKCI; NFKB2; MAP3K14; MAPK8; |
| | PRKD1; MAPK10; RELA; PRKCD; MAPK9; ABCB1; |
| | TRAF2; TLR4; TNF; INSR; IKBKG; RELB; MAP3K7; |
| | IL8; CHUK; NR1H2; TJP2; NFKB1; ESR1; |
| | SREBF1; FGFR4; JUN; IL1R1; PRKCA; IL6 |
| IGF-1 | IGF1; PRKCZ; ELK1; MAPK1; PTPN11; NEDD4; |
| Signaling | AKT2; PIK3CA; PRKCI; PTK2; FOS; |
| | PIK3CB; PIK3C3; MAPK8; |
| | IGF1R; IRS1; MAPK3; IGFBP7; KRAS; PIK3C2A; |
| | YWHAZ; PXN; RAF1; CASP9; MAP2K2; AKT1; |
| | PIK3R1; PDPK1; MAP2K1; IGFBP2; SFN; JUN; |
| | CYR61; AKT3; FOXO1; SRF; CTGF; RPS6KB1 |
| NRF2- | PRKCE; EP300; SOD2; |
| mediated | PRKCZ; MAPK1; SQSTM1; |
| Oxidative | NQO1; PIK3CA; PRKCI; FOS; |
| Stress | PIK3CB; PIK3C3; MAPK8; |
| Response | PRKD1; MAPK3; KRAS; |
| | PRKCD; GSTP1; MAPK9; FTL; |
| | NFE2L2; PIK3C2A; MAPK14; |
| | RAF1; MAP3K7; CREBBP; MAP2K2; |
| | AKT1; PIK3R1; MAP2K1; PPIB; JUN; KEAP1; |
| | GSK3B; ATF4; PRKCA; EIF2AK3; HSP90AA1 |
| Hepatic | EDN1; IGF1; KDR; FLT1; SMAD2; |
| Fibrosis/ | FGFR1; MET; PGF; SMAD3; EGFR; FAS; |
| Hepatic | CSF1; NFKB2; BCL2; MYH9; IGFIR; IL6R; |
| Stellate Cell | RELA; TLR4; PDGFRB; TNF; RELB; IL8; |
| Activation | PDGFRA; NFKB1; TGFBR1; SMAD4; VEGFA; BAX; |
| | IL1R1; CCL2; HGF; MMP1; STAT1; IL6; CTGF; MMP9 |
| PPAR | EP300; INS; TRAF6; PPARA; RXRA; MAPK1; IKBKB; |
| Signaling | NCOR2; FOS; NFKB2; MAP3K14; STAT5B; MAPK3; |
| | NRIP1; KRAS; PPARG; RELA; STAT5A; TRAF2; |
| | PPARGC1A; PDGFRB; TNF; INSR; RAF1; IKBKG; |
| | RELB; MAP3K7; CREBBP; MAP2K2; CHUK; |
| | PDGFRA; MAP2K1; NFKB1; JUN; IL1R1; HSP90AA1 |
| Fc Epsilon | PRKCE; RAC1; PRKCZ; LYN; MAPK1; RAC2; |
| RI Signaling | PTPN11; AKT2; PIK3CA; SYK; PRKCI; |
| | PIK3CB; PIK3C3; MAPK8; |
| | PRKD1; MAPK3; MAPK10; KRAS; MAPK13; PRKCD; |
| | MAPK9; PIK3C2A; BTK; MAPK14; TNF; RAF1; FYN; |
| | MAP2K2; AKT1; PIK3R1; PDPK1; MAP2K1; AKT3; |
| | VAV3; PRKCA |
| G-Protein | PRKCE; RAP1A; RGS16; |
| Coupled | MAPK1; GNAS; AKT2; IKBKB; |
| Receptor | PIK3CA; CREB1; GNAQ; NFKB2; CAMK2A; PIK3CB; |
| Signaling | PIK3C3; MAPK3; KRAS; RELA; SRC; PIK3C2A; |
| | RAF1; IKBKG; RELB; FYN; MAP2K2; AKT1; |
| | PIK3R1; CHUK; PDPK1; STAT3; MAP2K1; |
| | NFKB1; BRAF; ATF4; AKT3; PRKCA |
| Inositol | PRKCE; IRAK1; PRKAA2; EIF2AK2; PTEN; GRK6; |
| Phosphate | MAPK1; PLK1; AKT2; PIK3CA; |
| Metabolism | CDK8; PIK3CB; PIK3C3; |
| | MAPK8; MAPK3; PRKCD; PRKAA1; MAPK9; CDK2; |
| | PIM1; PIK3C2A; DYRK1A; MAP2K2; PIP5K1A; |
| | PIK3R1; MAP2K1; PAK3; ATM; TTK; CSNK1A1; |
| | BRAF; SGK |
| PDGF | EIF2AK2; ELK1; ABL2; MAPK1; |
| Signaling | PIK3CA; FOS; PIK3CB; |
| | PIK3C3; MAPK8; CAV1; ABL1; MAPK3; KRAS; SRC; |
| | PIK3C2A; PDGFRB; RAF1; MAP2K2; JAK1; JAK2; |
| | PIK3R1; PDGFRA; STAT3; SPHK1; MAP2K1; MYC; |
| | JUN; CRKL; PRKCA; SRF; STAT1; SPHK2 |
| VEGF | ACTN4; ROCK1; KDR; FLT1; ROCK2; MAPK1; PGF; |
| Signaling | AKT2; PIK3CA; ARNT; PTK2; |
| | BCL2; PIK3CB; PIK3C3; |
| | BCL2L1; MAPK3; KRAS; HIF1A; NOS3; PIK3C2A; |
| | PXN; RAF1; MAP2K2; ELAVL1; AKT1; PIK3R1; |
| | MAP2K1; SFN; VEGFA; AKT3; FOXO1; PRKCA |
| Natural | PRKCE; RAC1; PRKCZ; MAPK1; RAC2; PTPN11; |
| Killer Cell | KIR2DL3; AKT2; PIK3CA; SYK; PRKCI; PIK3CB; |
| Signaling | PIK3C3; PRKD1; MAPK3; KRAS; PRKCD; PTPN6; |
| | PIK3C2A; LCK; RAF1; FYN; MAP2K2; PAK4; AKT1; |
| | PIK3R1; MAP2K1; PAK3; AKT3; VAV3; PRKCA |
| Cell | HDAC4; SMAD3; SUV39H1; |
| Cycle: G1/S | HDAC5; CDKN1B; BTRC; |
| Checkpoint | ATR; ABL1; E2F1; HDAC2; HDAC7A; RB1; HDAC11; |
| Regulation | HDAC9; CDK2; E2F2; HDAC3; TP53; CDKN1A; |
| | CCND1; E2F4; ATM; RBL2; SMAD4; CDKN2A; |
| | MYC; NRG1; GSK3B; RBL1; HDAC6 |
| T Cell | RAC1; ELK1; MAPK1; IKBKB; CBL; PIK3CA; FOS; |
| Receptor | NFKB2; PIK3CB; PIK3C3; MAPK8; MAPK3; KRAS; |
| Signaling | RELA; PIK3C2A; BTK; LCK; RAF1; IKBKG; |
| | RELB; FYN; MAP2K2; PIK3R1; CHUK; MAP2K1; |
| | NFKB1; ITK; BCL10; JUN; VAV3 |
| Death | CRADD; HSPB1; BID; BIRC4; TBK1; IKBKB; FADD; |
| Receptor | FAS; NFKB2; BCL2; MAP3K14; MAPK8; RIPK1; |
| Signaling | CASP8; DAXX; TNFRSF10B; RELA; TRAF2; TNF; |
| | IKBKG; RELB; CASP9; CHUK; APAF1; NFKB1; |
| | CASP2; BIRC2; CASP3; BIRC3 |
| FGF | RAC1; FGFR1; MET; MAPKAPK2; MAPK1; PTPN11; |
| Signaling | AKT2; PIK3CA; CREB1; PIK3CB; PIK3C3; MAPK8; |
| | MAPK3; MAPK13; PTPN6; PIK3C2A; MAPK14; RAF1; |
| | AKT1; PIK3R1; STAT3; MAP2K1; FGFR4; |
| | CRKL; ATF4; AKT3; PRKCA; HGF |
| GM-CSF | LYN; ELK1; MAPK1; PTPN11; |
| Signaling | AKT2; PIK3CA; CAMK2A; |
| | STAT5B; PIK3CB; PIK3C3; GNB2L1; BCL2L1; |
| | MAPK3; ETS1; KRAS; RUNX1; PIM1; PIK3C2A; |
| | RAF1; MAP2K2; AKT1; JAK2; PIK3R1; |
| | STAT3; MAP2K1; CCND1; AKT3; STAT1 |
| Amyo- | BID; IGF1; RAC1; BIRC4; PGF; CAPNS1; CAPN2; |
| trophic | PIK3CA; BCL2; PIK3CB; PIK3C3; BCL2L1; CAPN1; |
| Lateral | PIK3C2A; TP53; CASP9; PIK3R1; RAB5A; CASP1; |
| Sclerosis | APAF1; VEGFA; BIRC2; BAX; AKT3; CASP3; BIRC3 |
| Signaling | |
| JAK/Stat | PTPN1; MAPK1; PTPN11; AKT2; PIK3CA; STAT5B; |
| Signaling | PIK3CB; PIK3C3; MAPK3; KRAS; SOCS1; STAT5A; |
| | PTPN6; PIK3C2A; RAF1; CDKN1A; MAP2K2; JAK1; |
| | AKT1; JAK2; PIK3R1; STAT3; MAP2K1; |
| | FRAP1; AKT3; STAT1 |
| Nicotinate | PRKCE; IRAK1; PRKAA2; EIF2AK2; GRK6; MAPK1; |
| and Nico- | PLK1; AKT2; CDK8; MAPK8; |
| tinamide | MAPK3; PRKCD; PRKAA1; |
| Metabolism | PBEF1; MAPK9; CDK2; PIM1; DYRK1A; MAP2K2; |
| | MAP2K1; PAK3; NT5E; TTK; CSNK1A1; BRAF; SGK |
| Chemokine | CXCR4; ROCK2; MAPK1; PTK2; FOS; CFL1; GNAQ; |
| Signaling | CAMK2A; CXCL12; MAPK8; MAPK3; KRAS; |
| | MAPK13; RHOA; CCR3; SRC; |
| | PPP1CC; MAPK14; NOX1; |
| | RAF1; MAP2K2; MAP2K1; JUN; CCL2; PRKCA |
| IL-2 | ELK1; MAPK1; PTPN11; AKT2; PIK3CA; SYK; FOS; |
| Signaling | STAT5B; PIK3CB; PIK3C3; MAPK8; MAPK3; KRAS; |
| | SOCS1; STAT5A; PIK3C2A; LCK; RAF1; MAP2K2; |
| | JAK1; AKT1; PIK3R1; MAP2K1; JUN; AKT3 |
| Synaptic | PRKCE; IGF1; PRKCZ; PRDX6; LYN; MAPK1; GNAS; |
| Long Term | PRKCI; GNAQ; PPP2R1A; IGF1R; PRKD1; MAPK3; |
| Depression | KRAS; GRN; PRKCD; NOS3; NOS2A; PPP2CA; |
| | YWHAZ; RAF1; MAP2K2; |
| | PPP2R5C; MAP2K1; PRKCA |
| Estrogen | TAF4B; EP300; CARM1; PCAF; MAPK1; NCOR2; |
| Receptor | SMARCA4; MAPK3; NRIP1; KRAS; SRC; NR3C1; |
| Signaling | HDAC3; PPARGC1A; RBM9; |
| | NCOA3; RAF1; CREBBP; |
| | MAP2K2; NCOA2; MAP2K1; PRKDC; ESR1; ESR2 |
| Protein | TRAF6; SMURF1; BIRC4; |
| Ubiquit- | BRCA1; UCHL1; NEDD4; |
| ination | CBL; UBE2I; BTRC; HSPA5; |
| Pathway | USP7; USP10; FBXW7; |
| | USP9X; STUB1; USP22; |
| | B2M; BIRC2; PARK2; USP8; |
| | USP1; VHL; HSP90AA1; BIRC3 |
| IL-10 | TRAF6; CCR1; ELK1; |
| Signaling | IKBKB; SP1; FOS; NFKB2; |
| | MAP3K14; MAPK8; MAPK13; |
| | RELA; MAPK14; TNF; |
| | IKBKG; RELB; MAP3K7; |
| | JAK1; CHUK; STAT3; NFKB1; |
| | JUN; IL1R1; IL6 |
| VDR/RXR | PRKCE; EP300; PRKCZ; |
| Activation | RXRA; GADD45A; HES1; |
| | NCOR2; SP1; PRKCI; |
| | CDKN1B; PRKD1; PRKCD; |
| | RUNX2; KLF4; YY1; |
| | NCOA3; CDKN1A; NCOA2; SPP1; |
| | LRP5; CEBPB; FOXO1; PRKCA |
| TGF-beta | EP300; SMAD2; SMURF1; |
| Signaling | MAPK1; SMAD3; SMAD1; |
| | FOS; MAPK8; MAPK3; |
| | KRAS; MAPK9; RUNX2; |
| | SERPINE1; RAF1; MAP3K7; |
| | CREBBP; MAP2K2; |
| | MAP2K1; TGFBR1; |
| | SMAD4; JUN; SMAD5 |
| Toll-like | IRAK1; EIF2AK2; MYD88; |
| Receptor | TRAF6; PPARA; ELK1; |
| Signaling | IKBKB; FOS; NFKB2; |
| | MAP3K14; MAPK8; MAPK13; |
| | RELA; TLR4; MAPK14; |
| | IKBKG; RELB; MAP3K7; CHUK; |
| | NFKB1; TLR2; JUN |
| p38 MAPK | HSPB1; IRAK1; TRAF6; |
| Signaling | MAPKAPK2; ELK1; FADD; FAS; |
| | CREB1; DDIT3; RPS6KA4; |
| | DAXX; MAPK13; TRAF2; |
| | MAPK14; TNF; MAP3K7; |
| | TGFBR1; MYC; ATF4; IL1R1; |
| | SRF; STAT1 |
| Neuro- | NTRK2; MAPK1; PTPN11; |
| trophin/ | PIK3CA; CREB1; FOS; |
| TRK | PIK3CB; PIK3C3; MAPK8; |
| Signaling | MAPK3; KRAS; PIK3C2A; |
| | RAF1; MAP2K2; AKT1; |
| | PIK3R1; PDPK1; MAP2K1; |
| | CDC42; JUN; ATF4 |
| FXR/RXR | INS; PPARA; FASN; RXRA; |
| Activation | AKT2; SDC1; MAPK8; |
| | APOB; MAPK10; PPARG; |
| | MTTP; MAPK9; PPARGC1A; |
| | TNF; CREBBP; AKT1; |
| | SREBF1; FGFR4; AKT3; FOXO1 |
| Synaptic | PRKCE; RAP1A; EP300; |
| Long Term | PRKCZ; MAPK1; CREB1; |
| Potentiation | PRKCI; GNAQ; CAMK2A; |
| | PRKD1; MAPK3; KRAS; |
| | PRKCD; PPP1CC; RAF1; |
| | CREBBP; MAP2K2; MAP2K1; |
| | ATF4; PRKCA |
| Calcium | RAP1A; EP300; HDAC4; |
| Signaling | MAPK1; HDAC5; CREB1; |
| | CAMK2A; MYH9; MAPK3; |
| | HDAC2; HDAC7A; HDAC11; |
| | HDAC9; HDAC3; CREBBP; |
| | CALR; CAMKK2; ATF4; HDAC6 |
| EGF | ELK1; MAPK1; EGFR; PIK3CA; |
| Signaling | FOS; PIK3CB; PIK3C3; |
| | MAPK8; MAPK3; PIK3C2A; |
| | RAF1; JAK1; PIK3R1; |
| | STAT3; MAP2K1; JUN; |
| | PRKCA; SRF; STAT1 |
| Hypoxia | EDN1; PTEN; EP300; |
| Signaling | NQO1; UBE2I; CREB1; ARNT; |
| in the | HIF1A; SLC2A4; NOS3; |
| Cardio- | TP53; LDHA; AKT1; ATM; |
| vascular | VEGFA; JUN; ATF4; |
| System | VHL; HSP90AA1 |
| LPS/IL-1 | IRAK1; MYD88; TRAF6; |
| Mediated | PPARA; RXRA; ABCA1; |
| Inhibition | MAPK8; ALDH1A1; GSTP1; |
| of RXR | MAPK9; ABCB1; TRAF2; |
| Function | TLR4; TNF; MAP3K7; |
| | NR1H2; SREBF1; JUN; IL1R1 |
| LXR/RXR | FASN; RXRA; NCOR2; |
| Activation | ABCA1; NFKB2; IRF3; RELA; |
| | NOS2A; TLR4; TNF; |
| | RELB; LDLR; NR1H2; NFKB1; |
| | SREBF1; IL1R1; CCL2; IL6; MMP9 |
| Amyloid | PRKCE; CSNK1E; MAPK1; |
| Processing | CAPNS1; AKT2; CAPN2; |
| | CAPN1; MAPK3; MAPK13; |
| | MAPT; MAPK14; AKT1; |
| | PSEN1; CSNK1A1; |
| | GSK3B; AKT3; APP |
| IL-4 | AKT2; PIK3CA; PIK3CB; |
| Signaling | PIK3C3; IRS1; KRAS; SOCS1; |
| | PTPN6; NR3C1; PIK3C2A; |
| | JAK1; AKT1; JAK2; PIK3R1; |
| | FRAP1; AKT3; RPS6KB1 |
| Cell Cycle: | EP300; PCAF; BRCA1; |
| G2/M DNA | GADD45A; PLK1; BTRC; |
| Damage | CHEK1; ATR; CHEK2; |
| Checkpoint | YWHAZ; TP53; CDKN1A; |
| Regulation | PRKDC; ATM; SFN; CDKN2A |
| Nitric Oxide | KDR; FLT1; PGF; AKT2; |
| Signaling | PIK3CA; PIK3CB; PIK3C3; |
| in the | CAV1; PRKCD; NOS3; |
| Cardio- | PIK3C2A; AKT1; PIK3R1; |
| vascular | VEGFA; AKT3; HSP90AA1 |
| System | |
| Purine | NME2; SMARCA4; MYH9; |
| Metabolism | RRM2; ADAR; EIF2AK4; |
| | PKM2; ENTPD1; RAD51; |
| | RRM2B; TJP2; RAD51C; |
| | NT5E; POLD1; NME1 |
| cAMP- | RAP1A; MAPK1; GNAS; |
| mediated | CREB1; CAMK2A; MAPK3; |
| Signaling | SRC; RAF1; MAP2K2; |
| | STAT3; MAP2K1; BRAF; ATF4 |
| Mito- | SOD2; MAPK8; CASP8; |
| chondrial | MAPK10; MAPK9; CASP9; |
| Dysfunction | PARK7; PSEN1; PARK2; APP; CASP3 |
| Notch | HES1; JAG1; NUMB; |
| Signaling | NOTCH4; ADAM17; NOTCH2; |
| | PSEN1; NOTCH3; NOTCH1; DLL4 |
| Endoplasmic | HSPA5; MAPK8; XBP1; |
| Reticulum | TRAF2; ATF6; CASP9; ATF4; |
| Stress | EIF2AK3; CASP3 |
| Pathway | NME2; AICDA; RRM2; |
| Pyrimidine | EIF2AK4; ENTPD1; RRM2B; |
| Metabolism | NT5E; POLD1; NME1 |
| Parkinson's | UCHL1; MAPK8; MAPK13; |
| Signaling | MAPK14; CASP9; PARK7; |
| | PARK2; CASP3 |
| Cardiac & | GNAS; GNAQ; PPP2R1A; |
| Beta | GNB2L1; PPP2CA; PPP1CC; |
| Adrenergic | PPP2R5C |
| Signaling | |
| Glycolysis/ | HK2; GCK; GPI; ALDH1A1; |
| Gluco- | PKM2; LDHA; HK1 |
| neogenesis | |
| Interferon | IRF1; SOCS1; JAK1; JAK2; |
| Signaling | IFITM1; STAT1; IFIT3 |
| Sonic | ARRB2; SMO; GLI2; |
| Hedgehog | DYRK1A; GLI1; GSK3B; DYRK1B |
| Signaling | |
| Glycero- | PLD1; GRN; GPAM; |
| phospholipid | YWHAZ; SPHK1; SPHK2 |
| Metabolism | |
| Phospholipid | PRDX6; PLD1; GRN; |
| Degradation | YWHAZ; SPHK1; SPHK2 |
| Tryptophan | SIAH2; PRMT5; NEDD4; |
| Metabolism | ALDH1A1; CYP1B1; SIAH1 |
| Lysine | SUV39H1; EHMT2; |
| Degradation | NSD1; SETD7; PPP2R5C |
| Nucleotide | ERCC5; ERCC4; |
| Excision | XPA; XPC; ERCC1 |
| Repair | |
| Pathway | |
| Starch and | UCHL1; HK2; |
| Sucrose | GCK; GPI; HK1 |
| Metabolism | |
| Aminosugars | NQO1; HK2; GCK; HK1 |
| Metabolism | |
| Arachidonic | PRDX6; GRN; |
| Acid | YWHAZ; CYP1B1 |
| Metabolism | |
| Circadian | CSNK1E; CREB1; |
| Rhythm | ATF4; NR1D1 |
| Signaling | |
| Coagulation | BDKRB1; F2R; |
| System | SERPINE1; F3 |
| Dopamine | PPP2R1A; PPP2CA; |
| Receptor | PPP1CC; PPP2R5C |
| Signaling | |
| Glutathione | IDH2; GSTP1; |
| Metabolism | ANPEP; IDH1 |
| Glycerolipid | ALDH1A1; GPAM; |
| Metabolism | SPHK1; SPHK2 |
| Linoleic | PRDX6; GRN; |
| Acid | YWHAZ; CYP1B1 |
| Metabolism | |
| Methionine | DNMT1; DNMT3B; |
| Metabolism | AHCY; DNMT3A |
| Pyruvate | GLO1; ALDH1A1; |
| Metabolism | PKM2; LDHA |
| Arginine | ALDH1A1; NOS3; NOS2A |
| and Proline | |
| Metabolism | |
| Eicosanoid | PRDX6; GRN; YWHAZ |
| Signaling | |
| Fructose and | HK2; GCK; HK1 |
| Mannose | |
| Metabolism | |
| Galactose | HK2; GCK; HK1I |
| Metabolism | |
| Stilbene, | PRDX6; PRDX1; TYR |
| Coumarine | |
| and Lignin | |
| Biosynthesis | |
| Antigen | CALR; B2M |
| Presentation | |
| Pathway | |
| Biosynthesis | NQO1; DHCR7 |
| of Steroids | |
| Butanoate | ALDH1A1; NLGN1 |
| Metabolism | |
| Citrate Cycle | IDH2; IDH1 |
| Fatty Acid | ALDH1A1; CYP1B1 |
| Metabolism | |
| Glycero- | PRDX6; CHKA |
| phospholipid | |
| Metabolism | |
| Histidine | PRMT5; ALDH1A1 |
| Metabolism | |
| Inositol | ERO1L; APEX1 |
| Metabolism | |
| Metabolism | GSTP1; CYP1B1 |
| of | |
| Xenobiotics | |
| by | |
| Cytochrome | |
| p450 | |
| Methane | PRDX6; PRDX1 |
| Metabolism | |
| Phenyl- | PRDX6; PRDX1 |
| alanine | |
| Metabolism | |
| Propanoate | ALDH1A1; LDHA |
| Metabolism | |
| Selenoamino | PRMT5; AHCY |
| Acid | |
| Metabolism | |
| Sphingolipid | SPHK1; SPHK2 |
| Metabolism | |
| Amino- | PRMT5 |
| phosphonate | |
| Metabolism | |
| Androgen | PRMT5 |
| and Estrogen | |
| Metabolism | |
| Ascorbate | ALDH1A1 |
| and Aldarate | |
| Metabolism | |
| Bile Acid | ALDH1A1 |
| Biosynthesis | |
| Cysteine | LDHA |
| Metabolism | |
| Fatty Acid | FASN |
| Biosynthesis | |
| Glutamate | GNB2L1 |
| Receptor | |
| Signaling | |
| NRF2- | PRDX1 |
| mediated | |
| Oxidative | |
| Stress | |
| Response | |
| Pentose | GP1 |
| Phosphate | |
| Pathway | |
| Pentose and | UCHL1 |
| Glucuronate | |
| Inter- | |
| conversions | |
| Retinol | ALDH1A1 |
| Metabolism | |
| Riboflavin | TYR |
| Metabolism | |
| Tyrosine | PRMT5, TYR |
| Metabolism | |
| Ubiquinone | PRMT5 |
| Biosynthesis | |
| Valine, | ALDH1A1 |
| Leucine and | |
| Isoleucine | |
| Degradation | |
| Glycine, | CHKA |
| Serine and | |
| Threonine | |
| Metabolism | |
| Lysine | ALDH1A1 |
| Degradation | |
| Pain/Taste | TRPM5; TRPAl |
| Pain | TRPM7; TRPC5; TRPC6; |
| | TRPC1; Cnr1; cnr2; Grk2; |
| | Trpa1; Pomc; Cgrp; Crf; Pka; |
| | Era; Nr2b; TRPM5; Prkaca; |
| | Prkacb; Prkar1a; Prkar2a |
| Mito- | AIF; CytC; SMAC (Diablo); |
| chondrial | Aifm-1; Aifm-2 |
| Function | |
| Develop- | BMP-4; Chordin (Chrd); |
| mental | Noggin (Nog); WNT (Wnt2; |
| Neurology | Wnt2b; Wnt3a; Wnt4; |
| | Wnt5a; Wnt6; Wnt7b; Wnt8b; |
| | Wnt9a; Wnt9b; Wnt10a; |
| | Wnt10b; Wnt16); beta-catenin; |
| | Dkk-1; Frizzled related proteins; |
| | Otx-2; Gbx2; FGF-8; Reelin; Dab1; |
| | unc-86 (Pou4f1 or Brn3a); Numb; Reln |

In an aspect, the present disclosure provides a method of individualized or personalized treatment of a genetic disease in a subject in need of such treatment comprising: (a) introducing one or more mutations ex vivo in a tissue, organ or a cell line, or in vivo in a transgenic non-human mammal, comprising delivering to cell(s) of the tissue, organ, cell or mammal a composition comprising the particle delivery system or the delivery system or the virus particle of any one of the above embodiment or the cell of any one of the above embodiment, wherein the specific mutations or precise sequence substitutions are or have been correlated to the genetic disease; (b) testing treatment(s) for the genetic disease on the cells to which the vector has been delivered that have the specific mutations or precise sequence substitutions correlated to the genetic disease; and (c) treating the subject based on results from the testing of treatment(s) of step (b).

### Infectious Diseases

In some embodiments, the composition, system,(s) or component(s) thereof can be used to diagnose, prognose, treat, and/or prevent an infectious disease caused by a microorganism, such as bacteria, virus, fungi, parasites, or combinations thereof.

In some embodiments, the system(s) or component(s) thereof can be capable of targeting specific microorganism within a mixed population. Exemplary methods of such techniques are described in e.g. Gomaa A A, Klumpe H E, Luo M L, Selle K, Barrangou R, Beisel C L. 2014. Programmable removal of bacterial strains by use of genome-targeting composition, systems, mBio 5:e00928-13; Citorik R J, Mimee M, Lu T K. 2014. Sequence-specific antimicrobials using efficiently delivered RNA-guided nucleases. Nat Biotechnol 32:1141-1145, the teachings of which can be adapted for use with the compositions, systems, and components thereof described herein.

In some embodiments, the composition, system,(s) and/or components thereof can be capable of targeting pathogenic and/or drug-resistant microorganisms, such as bacteria, virus, parasites, and fungi. In some embodiments, the composition, system,(s) and/or components thereof can be capable of targeting and modifying one or more polynucleotides in a pathogenic microorganism such that the microorganism is less virulent, killed, inhibited, or is otherwise rendered incapable of causing disease and/or infecting and/or replicating in a host cell.

In some embodiments, the pathogenic bacteria that can be targeted and/or modified by the composition, system,(s) and/or component(s) thereof described herein include, but are not limited to, those of the genus Actinomyces (e.g. A. israehi), Bacillus (e.g. B. anthracis, B. cereus), Bactereoides (e.g. B. fragilis), Bartonella (B. henselae, B. quintana), Bordetella (B. pertussis), Borrelia (e.g. B. burgdorferi, B. garinii, B. afzelii, and B. recurreentis), Brucella (e.g. B. abortus, B. canis, B. melitensis, and B. suis), Campylobacter (e.g. C. jejuni), Chlamydia (e.g. C. pneumoniae and C. trachomatis), Chlamydophila (e.g. C. psittaci), Clostridium (e.g. C. botulinum, C. difficile, C. perfringens. C. tetani), Corynebacterium (e.g. C. diptheriae), Enterococcus (e.g. E. faecalis, E. faecium), Ehrlichia (E. canis and E. chaffensis) Escherichia (e.g. E. coli), Francisella (e.g. F. tularensis), Haemophilus (e.g. H. influenzae), Helicobacter H. pylori), Klebsiella (E.g. K. pneumoniae), Legionella (e.g. L. pneumophila), Leptospira (e.g. L. interrogans, L. santarosai, L. weilii, L. noguchii), Listereia (e.g. L. monocytogeenes), Mycobacterium (e.g. M. leprae, M. tuberculosis, M. ulcerans), Mycoplasma (M. pneumoniae), Neisseria (N. gonorrhoeae and N. menigitidis), Nocardia (e.g. N. asteeroides), Pseudomonas (P. aeruginosa), Rickettsia (R. rickettsia), Salmonella (S. typhi and S. typhimurium), Shigella (S. sonnei and S. dysenteriae), Staphylococcus (S. aureus, S. epidermidis, and S. saprophyticus), Streeptococcus (S. agalactiaee, S. pneumoniae, S. pyogenes), Treponema (T. pallidum), Ureeaplasma (e.g. U. urealyticum), Vibrio (e.g. V. cholerae), Yersinia (e.g. Y. pestis, Y. enteerocolitica, and Y. pseudotuberculosis).

In some embodiments, the pathogenic virus that can be targeted and/or modified by the composition, system,(s) and/or component(s) thereof described herein include, but are not limited to, a double-stranded DNA virus, a partly double-stranded DNA virus, a single-stranded DNA virus, a positive single-stranded RNA virus, a negative single-stranded RNA virus, or a double stranded RNA virus. In some embodiments, the pathogenic virus can be from the family Adenoviridae (e.g. Adenovirus), Herpeesviridae (e.g. Herpes simplex, type 1, Herpes simplex, type 2, Varicella-zoster virus, Epstein-Barr virus, Human cytomegalovirus, Human herpesvirus, type 8), Papillomaviridae (e.g. Human papillomavirus), Polyomaviridae (e.g. BK virus, JC virus), Poxviridae (e.g. smallpox), Hepadnaviridae (e.g. Hepatitis B), Parvoviridae (e.g. Parvovirus B19), Astroviridae (e.g. Human astrovirus), Caliciviridae (e.g. Norwalk virus), Picornaviridae (e.g. coxsackievirus, hepatitis A virus, poliovirus, rhinovirus), Coronaviridae (e.g. Severe acute respiratory syndrome-related coronavirus, strains: Severe acute respiratory syndrome virus, Severe acute respiratory syndrome coronavirus 2 (COVID-19)), Flaviviridae (e.g. Hepatitis C virus, yellow fever virus, dengue virus, West Nile virus, TBE virus), Togaviridae (e.g. Rubella virus), Hepeviridae (e.g. Hepatitis E virus), Retroviridae (Human immunodeficiency virus (HIV)), Orthomyxoviridae (e.g. Influenza virus), Arenaviridae (e.g. Lassa virus), Bunyaviridae (e.g. Crimean-Congo hemorrhagic fever virus, Hantaan virus), Filoviridae (e.g. Ebola virus and Marburg virus), Paramyxoviridae (e.g. Measles virus, Mumps virus, Parainfluenza virus, Respiratory syncytial virus), Rhabdoviridae (Rabies virus), Hepatits D virus, Reoviridae (e.g. Rotavirus, Orbivirus, Coltivirus, Banna virus).

In some embodiments, the pathogenic fungi that can be targeted and/or modified by the composition, system,(s) and/or component(s) thereof described herein include, but are not limited to, those of the genus Candida (e.g. C. albicans), Aspergillus (e.g. A. fumigatus, A. flavus, A. clavatus), Cryptococcus (e.g. C. neoformans, C. gattii), Histoplasma (H. capsulatum), Pneumocystis (e.g. P. jiroveecii), Stachybotrys (e.g. S. chartarum).

In some embodiments, the pathogenic parasites that can be targeted and/or modified by the composition, system,(s) and/or component(s) thereof described herein include, but are not limited to, protozoa, helminths, and ectoparasites. In some embodiments, the pathogenic protozoa that can be targeted and/or modified by the composition, system,(s) and/or component(s) thereof described herein include, but are not limited to, those from the groups Sarcodina (e.g. ameba such as Entamoeba), Mastigophora (e.g. flagellates such as Giardia and Leishmania), Cilophora (e.g. ciliates such as Balantidum), and sporozoa (e.g. plasmodium and cryptosporidium). In some embodiments, the pathogenic helminths that can be targeted and/or modified by the composition, system,(s) and/or component(s) thereof described herein include, but are not limited to, flatworms (platyhelminths), thorny-headed worms (acanthoceephalins), and roundworms (nematodes). In some embodiments, the pathogenic ectoparasites that can be targeted and/or modified by the composition, system,(s) and/or component(s) thereof described herein include, but are not limited to, ticks, fleas, lice, and mites.

In some embodiments, the pathogenic parasite that can be targeted and/or modified by the composition, system,(s) and/or component(s) thereof described herein include, but are not limited to, Acanthamoeba spp., Balamuthia mandrillaris, Babesiosis spp. (e.g. Babesia B. divergens, B. bigemina, B. equi, B. microfti, B. duncani), Balantidiasis spp. (e.g. Balantidium coli), Blastocystis spp., Cryptosporidium spp., Cyclosporiasis spp. (e.g. Cyclospora cayetanensis), Dientamoebiasis spp. (e.g. Dientamoeba fragilis), Amoebiasis spp. (e.g. Entamoeba histolytica), Giardiasis spp. (e.g. Giardia lamblia), Isosporiasis spp. (e.g. Isospora belh), Leishmania spp., Naegleria spp. (e.g. Naegleria fowleri), Plasmodium spp. (e.g. Plasmodium falciparum, Plasmodium vivax, Plasmodium ovale curtisi, Plasmodium ovale wallikeri, Plasmodium malariae, Plasmodium knowlesi), Rhinosporidiosis spp. (e.g. Rhinosporidium seeberi), Sarcocystosis spp. (e.g. Sarcocystis bovihominis, Sarcocystis suihominis), Toxoplasma spp. (e.g. Toxoplasma gondii), Trichomonas spp. (e.g. Trichomonas vaginalis), Trypanosoma spp. (e.g. Trypanosoma brucei), Trypanosoma spp. (e.g. Trypanosoma cruzi), Tapeworm (e.g. Cestoda, Taenia multiceps, Taenia saginata, Taenia solium), Diphyllobothrium latum spp., Echinococcus spp. (e.g. Echinococcus granulosus, Echinococcus multilocularis, E. vogeli, E. oligarthrus), Hymenolepis spp. (e.g. Hymenolepis nana, Hymenolepis diminuta), Bertiella spp. (e.g. Bertiella mucronata, Bertiella studeri), Spirometra (e.g. Spirometra erinaceieuropaei), Clonorchis spp. (e.g. Clonorchis sinensis; Clonorchis viverrini), Dicrocoelium spp. (e.g. Dicrocoelium dendriticum), Fasciola spp. (e.g. Fasciola hepatica, Fasciola gigantica), Fasciolopsis spp. (e.g. Fasciolopsis buski), Metagonimus spp. (e.g. Metagonimus yokogawai), Metorchis spp. (e.g. Metorchis conjunctus), Opisthorchis spp. (e.g. Opisthorchis viverrini, Opisthorchis felineus), Clonorchis spp. (e.g. Clonorchis sinensis), Paragonimus spp. (e.g. Paragonimus westermani; Paragonimus africanus; Paragonimus caliensis; Paragonimus kellicotti; Paragonimus skrjabini; Paragonimus uterobilateralis), Schistosoma sp., Schistosoma spp. (e.g. Schistosoma mansoni, Schistosoma haematobium, Schistosoma japonicum, Schistosoma mekongi, and Schistosoma intercalatum), Echinostoma spp. (e.g. E. echinatum), Trichobilharzia spp. (e.g. Trichobilharzia regent), Ancylostoma spp. (e.g. Ancylostoma duodenale), Necator spp. (e.g. Necator americanus), Angiostrongylus spp., Anisakis spp., Ascaris spp. (e.g. Ascaris lumbricoides), Baylisascaris spp. (e.g. Baylisascaris procyonis), Brugia spp. (e.g. Brugia malayi, Brugia timori), Dioctophyme spp. (e.g. Dioctophyme renale), Dracunculus spp. (e.g. Dracunculus medinensis), Enterobius spp. (e.g. Enterobius vermicularis, Enterobius gregorii), Gnathostoma spp. (e.g. Gnathostoma spinigerum, Gnathostoma hispidum), Halicephalobus spp. (e.g. Halicephalobus gingivalis), Loa loa spp. (e.g. Loa boa filaria), Mansonella spp. (e.g. Mansonella streptocerca), Onchocerca spp. (e.g. Onchocerca volvulus), Strongyloides spp. (e.g. Strongyloides stercoralis), Thelazia spp. (e.g. Thelazia californiensis, Thelazia callipaeda), Toxocara spp. (e.g. Toxocara canis, Toxocara cati, Toxascaris leonine), Trichinella spp. (e.g. Trichinella spiralis, Trichinella britovi, Trichinella nelsoni, Trichinella nativa), Trichuris spp. (e.g. Trichuris trichiura, Trichuris vulpis), Wuchereria spp. (e.g. Wuchereria bancrofti), Dermatobia spp. (e.g. Dermatobia hominis), Tunga spp. (e.g. Tunga penetrans), Cochliomyia spp. (e.g. Cochliomyia hominivorax), Linguatula spp. (e.g. Linguatula serrata), Archiacanthocephala sp., Moniliformis sp. (e.g. Moniliformis moniliformis), Pediculus spp. (e.g. Pediculus humanus capitis, Pediculus humanus humanus), Pthirus spp. (e.g. Pthirus pubis), Arachnida spp. (e.g. Trombiculidae, Ixodidae, Argaside), Siphonaptera spp (e.g. Siphonaptera: Pulicinae), Cimicidae spp. (e.g. Cimex lectularius and Cimex hemipterus), Diptera spp., Demodex spp. (e.g. Demodex folliculorum/brevis/canis), Sarcoptes spp. (e.g. Sarcoptes scabiei), Dermanyssus spp. (e.g. Dermanyssus gallinae), Ornithonyssus spp. (e.g. Ornithonyssus sylviarum, Ornithonyssus bursa, Ornithonyssus bacoti), Laelaps spp. (e.g. Laelaps echidnina), Liponyssoides spp. (e.g. Liponyssoides sanguineus).

In some embodiments the gene targets can be any of those as set forth in Table 1 of Strich and Chertow. 2019. J. Clin. Microbio. 57:4 e01307-18, which is incorporated herein as if expressed in its entirety herein.

In some embodiments, the method can include delivering a composition, system, and/or component thereof to a pathogenic organism described herein, allowing the composition, system, and/or component thereof to specifically bind and modify one or more targets in the pathogenic organism, whereby the modification kills, inhibits, reduces the pathogenicity of the pathogenic organism, or otherwise renders the pathogenic organism non-pathogenic. In some embodiments, delivery of the composition, system, occurs in vivo (i.e. in the subject being treated). In some embodiments occurs by an intermediary, such as microorganism or phage that is non-pathogenic to the subject but is capable of transferring polynucleotides and/or infecting the pathogenic microorganism. In some embodiments, the intermediary microorganism can be an engineered bacteria, virus, or phage that contains the composition, system,(s) and/or component(s) thereof and/or vectors and/or vector systems. The method can include administering an intermediary microorganism containing the composition, system,(s) and/or component(s) thereof and/or vectors and/or vector systems to the subject to be treated. The intermediary microorganism can then produce the compositions and/or component thereof or transfer a composition, system, polynucleotide to the pathogenic organism. In embodiments, where the compositions and/or component thereof, vector, or vector system is transferred to the pathogenic microorganism, the composition, system, or component thereof is then produced in the pathogenic microorganism and modifies the pathogenic microorganism such that it is less virulent, killed, inhibited, or is otherwise rendered incapable of causing disease and/or infecting and/or replicating in a host or cell thereof.

In some embodiments, where the pathogenic microorganism inserts its genetic material into the host cell's genome (e.g. a virus), the composition, system, can be designed such that it modifies the host cell's genome such that the viral DNA or cDNA cannot be replicated by the host cell's machinery into a functional virus. In some embodiments, where the pathogenic microorganism inserts its genetic material into the host cell's genome (e.g. a virus), the composition, system, can be designed such that it modifies the host cell's genome such that the viral DNA or cDNA is deleted from the host cell's genome.

It will be appreciated that inhibiting or killing the pathogenic microorganism, the disease and/or condition that its infection causes in the subject can be treated or prevented. Thus, also provided herein are methods of treating and/or preventing one or more diseases or symptoms thereof caused by any one or more pathogenic microorganisms, such as any of those described herein.

### Mitochondrial Diseases

Some of the most challenging mitochondrial disorders arise from mutations in mitochondrial DNA (mtDNA), a high copy number genome that is maternally inherited. In some embodiments, mtDNA mutations can be modified using a composition, system, described herein. In some embodiments, the mitochondrial disease that can be diagnosed, prognosed, treated, and/or prevented can be MELAS (mitochondrial myopathy encephalopathy, and lactic acidosis and stroke-like episodes), CPEO/PEO (chronic progressive external ophthalmoplegia syndrome/progressive external ophthalmoplegia), KSS (Kearns-Sayre syndrome), MIDD (maternally inherited diabetes and deafness), MERRF (myoclonic epilepsy associated with ragged red fibers), NIDDM (noninsulin-dependent diabetes mellitus), LHON (Leber hereditary optic neuropathy), LS (Leigh Syndrome) an aminoglycoside induced hearing disorder, NARP (neuropathy, ataxia, and pigmentary retinopathy), Extrapyramidal disorder with akinesia-rigidity, psychosis and SNHL, Nonsyndromic hearing loss a cardiomyopathy, an encephalomyopathy, Pearson's syndrome, or a combination thereof.

In some embodiments, the mtDNA of a subject can be modified in vivo or ex vivo. In some embodiments, where the mtDNA is modified ex vivo, after modification the cells containing the modified mitochondria can be administered back to the subject. In some embodiments, the composition, system, or component thereof can be capable of correcting an mtDNA mutation, or a combination thereof.

In some embodiments, at least one of the one or more mtDNA mutations is selected from the group consisting of: A3243G, C3256T, T3271C, G1019A, A1304T, A15533G, C1494T, C4467A, T1658C, G12315A, A3421G, A8344G, T8356C, G8363A, A13042T, T3200C, G3242A, A3252G, T3264C, G3316A, T3394C, T14577C, A4833G, G3460A, G9804A, G11778A, G14459A, A14484G, G15257A, T8993C, T8993G, G10197A, G13513A, T1095C, C1494T, A1555G, G1541A, C1634T, A3260G, A4269G, T7587C, A8296G, A8348G, G8363A, T9957C, T9997C, G12192A, C12297T, A14484G, G15059A, duplication of CCCCCTCCCC-tandem repeats at positions 305-314 and/or 956-965, deletion at positions from 8,469-13,447, 4,308-14,874, and/or 4,398-14,822, 961ins/delC, the mitochondrial common deletion (e.g. mtDNA 4,977 bp deletion), and combinations thereof.

In some embodiments, the mitochondrial mutation can be any mutation as set forth in or as identified by use of one or more bioinformatic tools available at Mitomap available at mitomap.org. Such tools include, but are not limited to, “Variant Search, aka Market Finder”, Find Sequences for Any Haplogroup, aka “Sequence Finder”, “Variant Info”, “POLG Pathogenicity Prediction Server”, “MITOMASTER”, “Allele Search”, “Sequence and Variant Downloads”, “Data Downloads”. MitoMap contains reports of mutations in mtDNA that can be associated with disease and maintains a database of reported mitochondrial DNA Base Substitution Diseases: rRNA/tRNA mutations.

In some embodiments, the method includes delivering a composition, system, and/or a component thereof to a cell, and more specifically one or more mitochondria in a cell, allowing the composition, system, and/or component thereof to modify one or more target polynucleotides in the cell, and more specifically one or more mitochondria in the cell. The target polynucleotides can correspond to a mutation in the mtDNA, such as any one or more of those described herein. In some embodiments, the modification can alter a function of the mitochondria such that the mitochondria functions normally or at least is/are less dysfunctional as compared to an unmodified mitochondria. Modification can occur in vivo or ex vivo. Where modification is performed ex vivo, cells containing modified mitochondria can be administered to a subject in need thereof in an autologous or allogenic manner.

### Microbiome Modification

Microbiomes play important roles in health and disease. For example, the gut microbiome can play a role in health by controlling digestion, preventing growth of pathogenic microorganisms and have been suggested to influence mood and emotion. Imbalanced microbiomes can promote disease and are suggested to contribute to weight gain, unregulated blood sugar, high cholesterol, cancer, and other disorders. A healthy microbiome has a series of joint characteristics that can be distinguished from non-healthy individuals, thus detection and identification of the disease-associated microbiome can be used to diagnose and detect disease in an individual. The compositions, systems, and components thereof can be used to screen the microbiome cell population and be used to identify a disease associated microbiome. Cell screening methods utilizing compositions, systems, and components thereof are described elsewhere herein and can be applied to screening a microbiome, such as a gut, skin, vagina, and/or oral microbiome, of a subject.

In some embodiments, the microbe population of a microbiome in a subject can be modified using a composition, system, and/or component thereof described herein. In some embodiments, the composition, system, and/or component thereof can be used to identify and select one or more cell types in the microbiome and remove them from the microbiome population. Exemplary methods of selecting cells using a composition, system, and/or component thereof are described elsewhere herein. In this way the make-up or microorganism profile of the microbiome can be altered. In some embodiments, the alteration causes a change from a diseased microbiome composition to a healthy microbiome composition. In this way the ratio of one type or species of microorganism to another can be modified, such as going from a diseased ratio to a healthy ratio. In some embodiments, the cells selected are pathogenic microorganisms.

In some embodiments, the compositions and systems described herein can be used to modify a polynucleotide in a microorganism of a microbiome in a subject. In some embodiments, the microorganism is a pathogenic microorganism. In some embodiments, the microorganism is a commensal and non-pathogenic microorganism. Methods of modifying polynucleotides in a cell in the subject are described elsewhere herein and can be applied to these embodiments.

### Models of Diseases and Conditions

In an aspect, the present disclosure provides a method of modeling a disease associated with a genomic locus in a eukaryotic organism or a non-human organism comprising manipulation of a target sequence within a coding, non-coding or regulatory element of said genomic locus comprising delivering a non-naturally occurring or engineered composition comprising a viral vector system comprising one or more viral vectors operably encoding a composition for expression thereof, wherein the composition comprises particle delivery system or the delivery system or the virus particle of any one of the above embodiments or the cell of any one of the above embodiment.

In one aspect, the present disclosure provides a method of generating a model eukaryotic cell that can include one or more a mutated disease genes and/or infectious microorganisms. In some embodiments, a disease gene is any gene associated an increase in the risk of having or developing a disease. In some embodiments, the method includes (a) introducing one or more vectors into a eukaryotic cell, wherein the one or more vectors comprise a composition, system, and/or component thereof and/or a vector or vector system that is capable of driving expression of a composition, system, and/or component thereof including, but not limited to: a guide sequence optionally linked to a tracr mate sequence, a tracr sequence, one or more nucleic acid-guided nucleases, and combinations thereof and (b) allowing a composition, system, or complex to bind to one or more target polynucleotides, e.g., to effect cleavage, nicking, or other modification of the target polynucleotide within said disease gene, wherein the composition, system, or complex is composed of one or more nucleic acid-guided nuclease complexed with (1) one or more guide sequences that is/are hybridized to the target sequence(s) within the target polynucleotide(s), and optionally (2) the tracr mate sequence(s) that is/are hybridized to the tracr sequence(s), thereby generating a model eukaryotic cell comprising one or more mutated disease gene(s). Thus, in some embodiments the composition and system, contains nucleic acid molecules for and drives expression of one or more of: a nucleic acid-guided nuclease, a guide sequence linked to a tracr mate sequence, and a tracr sequence and/or a Homologous Recombination template and/or a stabilizing ligand if the nucleic acid-guided nuclease has a destabilization domain. In some embodiments, said cleavage comprises cleaving one or two strands at the location of the target sequence by the nucleic acid-guided nuclease. In some embodiments, nicking comprises nicking one or two strands at the location of the target sequence by the nucleic acid-guided nuclease. In some embodiments, said cleavage or nicking results in modified transcription of a target polynucleotide. In some embodiments, modification results in decreased transcription of the target polynucleotide. In some embodiments, the method further comprises repairing said cleaved or nicked target polynucleotide by homologous recombination with an recombination template polynucleotide, wherein said repair results in a mutation comprising an insertion, deletion, or substitution of one or more nucleotides of said target polynucleotide. In some embodiments, said mutation results in one or more amino acid changes in a protein expression from a gene comprising the target sequence.

The disease modeled can be any disease with a genetic or epigenetic component. In some embodiments, the disease modeled can be any as discussed elsewhere herein.

### In Situ Disease Detection

The compositions, systems, and/or components thereof can be used for diagnostic methods of detection such as in CASFISH (see e.g. Deng et al. 2015. PNAS USA 112(38): 11870-11875), CRISPR-Live FISH (see e.g. Wang et al. 2020. Science; 365(6459):1301-1305), sm-FISH (Lee and Jefcoate. 2017. Front. Endocrinol. doi.org/10.3389/fendo.2017.00289), sequential FISH CRISPRainbow (Ma et al. Nat Biotechnol, 34 (2016), pp. 528-530), CRISPR-Sirius (Nat Methods, 15 (2018), pp. 928-931), Casilio (Cheng et al. Cell Res, 26 (2016), pp. 254-257), Halo-Tag based genomic loci visualization techniques (e.g. Deng et al. 2015. PNAS USA 112(38): 11870-11875; Knight et al., Science, 350 (2015), pp. 823-826), RNA-aptamer based methods (e.g. Ma et al., J Cell Biol, 214 (2016), pp. 529-537), molecular beacon-based methods (e.g. Zhao et al. Biomaterials, 100 (2016), pp. 172-183; Wu et al. Nucleic Acids Res (2018)), Quantum Dot-based systems (e.g. Ma et al. Anal Chem, 89 (2017), pp. 12896-12901), multiplexed methods (e.g. Ma et al., Proc Natl Acad Sci USA, 112 (2015), pp. 3002-3007; Fu et al. Nat Commun, 7 (2016), p. 11707; Ma et al. Nat Biotechnol, 34 (2016), pp. 528-530; Shao et al. Nucleic Acids Res, 44 (2016), Article e86); Wang et al. Sci Rep, 6 (2016), p. 26857), c, and other in situ CRISPR-hybridization based methods (e.g. Chen et al. Cell, 155 (2013), pp. 1479-1491; Gu et al. Science, 359 (2018), pp. 1050-1055; Tanebaum et al. Cell, 159 (2014), pp. 635-646; Ye et al. Protein Cell, 8 (2017), pp. 853-855; Chen et al. Nat Commun, 9 (2018), p. 5065; Shao et al. ACS Synth Biol (2017); Fu et al. Nat Commun, 7 (2016), p. 11707; Shao et al. Nucleic Acids Res, 44 (2016), Article e86; Wang et al., Sci Rep, 6 (2016), p. 26857), all of which are incorporated by reference herein as if expressed in their entirety and whose teachings can be adapted to the compositions, systems, and components thereof described herein in view of the description herein.

In some embodiments, the composition, system, or component thereof can be used in a detection method, such as an in situ detection method described herein. In some embodiments, the composition, system, or component thereof can include a catalytically inactivate nucleic acid-guided nuclease described herein and use this system in detection methods such as fluorescence in situ hybridization (FISH) or any other described herein. In some embodiments, the inactivated nucleic acid-guided nuclease, which lacks the ability to produce DNA double-strand breaks may be fused with a marker, such as fluorescent protein, such as the enhanced green fluorescent protein (eEGFP) and co-expressed with small guide RNAs to target pericentric, centric and telomeric repeats in vivo. The dead nucleic acid-guided nuclease or system thereof can be used to visualize both repetitive sequences and individual genes in the human genome. Such new applications of labelled dead nucleic acid-guided nuclease and compositions, systems, thereof can be important in imaging cells and studying the functional nuclear architecture, especially in cases with a small nucleus volume or complex 3-D structures.

### Cell Selection

In some embodiments, the compositions, systems, and/or components thereof described herein can be used in a method to screen and/or select cells. In some embodiments, composition, system,-based screening/selection method can be used to identify diseased cells in a cell population. In some embodiments, selection of the cells results in a modification in the cells such that the selected cells die. In this way, diseased cells can be identified, and removed from the healthy cell population. In some embodiments, the diseased cells can be a cancer cell, pre-cancerous cell, a virus or other pathogenic organism infected cells, or otherwise abnormal cell. In some embodiments, the modification can impart another detectable change in the cells to be selected (e.g. a functional change and/or genomic barcode) that facilitates selection of the desired cells. In some embodiments a negative selection scheme can be used to obtain a desired cell population. In these embodiments, the cells to be selected against are modified, thus can be removed from the cell population based on their death or identification or sorting based the detectable change imparted on the cells. Thus, in these embodiments, the remaining cells after selection are the desired cell population.

In some embodiments, a method of selecting one or more cell(s) containing a polynucleotide modification can include: introducing one or more composition, system,(s) and/or components thereof, and/or vectors or vector systems into the cell(s), wherein the composition, system,(s) and/or components thereof, and/or vectors or vector systems contains and/or is capable of expressing one or more of: a nucleic acid-guided nuclease, a guide sequence optionally linked to a tracr mate sequence, a tracr sequence, and an recombination template; wherein, for example that which is being expressed is within and expressed in vivo by the composition, system, vector or vector system and/or the recombination template comprises the one or more mutations that abolish nucleic acid-guided nuclease cleavage; allowing homologous recombination of the recombination template with the target polynucleotide in the cell(s) to be selected; allowing a composition, system, or complex to bind to a target polynucleotide to effect cleavage of the target polynucleotide within said gene, wherein the AAV-complex comprises the nucleic acid-guided nuclease complexed with (1) the guide sequence that is hybridized to the target sequence within the target polynucleotide, and (2) the tracr mate sequence that is hybridized to the tracr sequence, wherein binding of the complex to the target polynucleotide induces cell death or imparts some other detectable change to the cell, thereby allowing one or more cell(s) in which one or more mutations have been introduced to be selected. In some embodiments, the cell to be selected may be a eukaryotic cell. In some embodiments, the cell to be selected may be a prokaryotic cell. Selection of specific cells via the methods herein can be performed without requiring a selection marker or a two-step process that may include a counter-selection system.

### Therapeutic Agent Development

The compositions, systems, and components thereof described herein can be used to develop nucleic acid-guided nuclease-based biologically active agents, such as small molecule therapeutics. Thus, described herein are methods for developing a biologically active agent that modulates a cell function and/or signaling event associated with a disease and/or disease gene. In some embodiments, the method comprises (a) contacting a test compound with a diseased cell and/or a cell containing a disease gene cell; and (b) detecting a change in a readout that is indicative of a reduction or an augmentation of a cell signaling event or other cell functionality associated with said disease or disease gene, thereby developing said biologically active agent that modulates said cell signaling event or other functionality associated with said disease gene. In some embodiments, the diseased cell is a model cell described elsewhere herein. In some embodiments, the diseased cell is a diseased cell isolated from a subject in need of treatment. In some embodiments, the test compound is a small molecule agent. In some embodiments, test compound is a small molecule agent. In some embodiments, the test compound is a biologic molecule agent.

In some embodiments, the method involves developing a therapeutic based on the composition, system, described herein. In particular embodiments, the therapeutic comprises a nucleic acid-guided nuclease and/or a guide RNA capable of hybridizing to a target sequence of interest. In particular embodiments, the therapeutic is a vector or vector system that can contain a) a first regulatory element operably linked to a nucleotide sequence encoding the nucleic acid-guided nuclease; and b) a second regulatory element operably linked to one or more nucleotide sequences encoding one or more nucleic acid molecules comprising a guide RNA comprising a guide sequence, a direct repeat sequence; wherein components (a) and (b) are located on same or different vectors. In particular embodiments, the biologically active agent is a composition comprising a delivery system operably configured to deliver composition, system, or components thereof, and/or or one or more polynucleotide sequences, vectors, or vector systems containing or encoding said components into a cell and capable of forming a complex with the components of the composition and system herein, and wherein said complex is operable in the cell. In some embodiments, the complex can include the nucleic acid-guided nuclease as described herein, guide RNA comprising the guide sequence, and a direct repeat sequence. In any such compositions, the delivery system can be a yeast system, a lipofection system, a microinjection system, a biolistic system, virosomes, liposomes, immunoliposomes, polycations, lipid:nucleic acid conjugates or artificial virions, or any other system as described herein. In particular embodiments, the delivery is via a particle, a nanoparticle, a lipid or a cell penetrating peptide (CPP).

Also described herein are methods for developing or designing a composition, system, optionally a composition, system, based therapy or therapeutic, comprising (a) selecting for a (therapeutic) locus of interest gRNA target sites, wherein said target sites have minimal sequence variation across a population, and from said selected target sites subselecting target sites, wherein a gRNA directed against said target sites recognizes a minimal number of off-target sites across said population, or (b) selecting for a (therapeutic) locus of interest gRNA target sites, wherein said target sites have minimal sequence variation across a population, or selecting for a (therapeutic) locus of interest gRNA target sites, wherein a gRNA directed against said target sites recognizes a minimal number of off-target sites across said population, and optionally estimating the number of (sub)selected target sites needed to treat or otherwise modulate or manipulate a population, and optionally validating one or more of the (sub)selected target sites for an individual subject, optionally designing one or more gRNA recognizing one or more of said (sub)selected target sites.

In some embodiments, the method for developing or designing a gRNA for use in a composition, system, optionally a composition, system, based therapy or therapeutic, can include (a) selecting for a (therapeutic) locus of interest gRNA target sites, wherein said target sites have minimal sequence variation across a population, and from said selected target sites subselecting target sites, wherein a gRNA directed against said target sites recognizes a minimal number of off-target sites across said population, or (b) selecting for a (therapeutic) locus of interest gRNA target sites, wherein said target sites have minimal sequence variation across a population, or selecting for a (therapeutic) locus of interest gRNA target sites, wherein a gRNA directed against said target sites recognizes a minimal number of off-target sites across said population, and optionally estimating the number of (sub)selected target sites needed to treat or otherwise modulate or manipulate a population, optionally validating one or more of the (sub)selected target sites for an individual subject, optionally designing one or more gRNA recognizing one or more of said (sub)selected target sites.

In some embodiments, thee method for developing or designing a composition, system, optionally a composition, system, based therapy or therapeutic in a population, can include (a) selecting for a (therapeutic) locus of interest gRNA target sites, wherein said target sites have minimal sequence variation across a population, and from said selected target sites subselecting target sites, wherein a gRNA directed against said target sites recognizes a minimal number of off-target sites across said population, or (b) selecting for a (therapeutic) locus of interest gRNA target sites, wherein said target sites have minimal sequence variation across a population, or selecting for a (therapeutic) locus of interest gRNA target sites, wherein a gRNA directed against said target sites recognizes a minimal number of off-target sites across said population, and optionally estimating the number of (sub)selected target sites needed to treat or otherwise modulate or manipulate a population, optionally validating one or more of the (sub)selected target sites for an individual subject, optionally designing one or more gRNA recognizing one or more of said (sub)selected target sites.

In some embodiments the method for developing or designing a gRNA for use in a composition, system, optionally a composition, system, based therapy or therapeutic in a population, can include (a) selecting for a (therapeutic) locus of interest gRNA target sites, wherein said target sites have minimal sequence variation across a population, and from said selected target sites subselecting target sites, wherein a gRNA directed against said target sites recognizes a minimal number of off-target sites across said population, or (b) selecting for a (therapeutic) locus of interest gRNA target sites, wherein said target sites have minimal sequence variation across a population, or selecting for a (therapeutic) locus of interest gRNA target sites, wherein a gRNA directed against said target sites recognizes a minimal number of off-target sites across said population, and optionally estimating the number of (sub)selected target sites needed to treat or otherwise modulate or manipulate a population, optionally validating one or more of the (sub)selected target sites for an individual subject, optionally designing one or more gRNA recognizing one or more of said (sub)selected target sites.

In some embodiments, the method for developing or designing a composition, system, such as a composition, system, based therapy or therapeutic, optionally in a population; or for developing or designing a gRNA for use in a composition, system, optionally a composition, system, based therapy or therapeutic, optionally in a population, can include selecting a set of target sequences for one or more loci in a target population, wherein the target sequences do not contain variants occurring above a threshold allele frequency in the target population (i.e. platinum target sequences); removing from said selected (platinum) target sequences any target sequences having high frequency off-target candidates (relative to other (platinum) targets in the set) to define a final target sequence set; preparing one or more, such as a set of compositions, systems, based on the final target sequence set, optionally wherein a number of composition prepared is based (at least in part) on the size of a target population.

In certain embodiments, off-target candidates/off-targets, PAM restrictiveness, target cleavage efficiency, or effector protein specificity is identified or determined using a sequencing-based double-strand break (DSB) detection assay, such as described herein elsewhere. In certain embodiments, off-target candidates/off-targets are identified or determined using a sequencing-based double-strand break (DSB) detection assay, such as described herein elsewhere. In certain embodiments, off-targets, or off target candidates have at least 1, preferably 1-3, mismatches or (distal) PAM mismatches, such as 1 or more, such as 1, 2, 3, or more (distal) PAM mismatches. In certain embodiments, sequencing-based DSB detection assay comprises labeling a site of a DSB with an adapter comprising a primer binding site, labeling a site of a DSB with a barcode or unique molecular identifier, or combination thereof, as described herein elsewhere.

It will be understood that the guide sequence of the gRNA is 100% complementary to the target site, i.e. does not comprise any mismatch with the target site. It will be further understood that “recognition” of an (off-)target site by a gRNA presupposes composition, system, functionality, i.e. an (off-)target site is only recognized by a gRNA if binding of the gRNA to the (off-)target site leads to composition, system, activity (such as induction of single or double strand DNA cleavage, transcriptional modulation, etc.).

In certain embodiments, the target sites having minimal sequence variation across a population are characterized by absence of sequence variation in at least 99%, preferably at least 99.9%, more preferably at least 99.99% of the population. In certain embodiments, optimizing target location comprises selecting target sequences or loci having an absence of sequence variation in at least 99%, %, preferably at least 99.9%, more preferably at least 99.99% of a population. These targets are referred to herein elsewhere also as “platinum targets”. In certain embodiments, said population comprises at least 1000 individuals, such as at least 5000 individuals, such as at least 10000 individuals, such as at least 50000 individuals.

In certain embodiments, the off-target sites are characterized by at least one mismatch between the off-target site and the gRNA. In certain embodiments, the off-target sites are characterized by at most five, preferably at most four, more preferably at most three mismatches between the off-target site and the gRNA. In certain embodiments, the off-target sites are characterized by at least one mismatch between the off-target site and the gRNA and by at most five, preferably at most four, more preferably at most three mismatches between the off-target site and the gRNA.

In certain embodiments, said minimal number of off-target sites across said population is determined for high-frequency haplotypes in said population. In certain embodiments, said minimal number of off-target sites across said population is determined for high-frequency haplotypes of the off-target site locus in said population. In certain embodiments, said minimal number of off-target sites across said population is determined for high-frequency haplotypes of the target site locus in said population. In certain embodiments, the high-frequency haplotypes are characterized by occurrence in at least 0.1% of the population.

In certain embodiments, the number of (sub)selected target sites needed to treat a population is estimated based on based low frequency sequence variation, such as low frequency sequence variation captured in large scale sequencing datasets. In certain embodiments, the number of (sub)selected target sites needed to treat a population of a given size is estimated.

In certain embodiments, the method further comprises obtaining genome sequencing data of a subject to be treated; and treating the subject with a composition, system, selected from the set of compositions, systems, wherein the composition, system, selected is based (at least in part) on the genome sequencing data of the individual. In certain embodiments, the ((sub)selected) target is validated by genome sequencing, preferably whole genome sequencing.

In certain embodiments, target sequences or loci as described herein are (further) selected based on optimization of one or more parameters, such as PAM type (natural or modified), PAM nucleotide content, PAM length, target sequence length, PAM restrictiveness, target cleavage efficiency, and target sequence position within a gene, a locus or other genomic region. Methods of optimization are discussed in greater detail elsewhere herein.

In certain embodiments, target sequences or loci as described herein are (further) selected based on optimization of one or more of target loci location, target length, target specificity, and PAM characteristics. As used herein, PAM characteristics may comprise for instance PAM sequence, PAM length, and/or PAM GC contents. In certain embodiments, optimizing PAM characteristics comprises optimizing nucleotide content of a PAM. In certain embodiments, optimizing nucleotide content of PAM is selecting a PAM with a motif that maximizes abundance in the one or more target loci, minimizes mutation frequency, or both. Minimizing mutation frequency can for instance be achieved by selecting PAM sequences devoid of or having low or minimal CpG.

In certain embodiments, the effector protein for each composition and system, in the set of compositions, systems, is selected based on optimization of one or more parameters selected from the group consisting of; effector protein size, ability of effector protein to access regions of high chromatin accessibility, degree of uniform enzyme activity across genomic targets, epigenetic tolerance, mismatch/budge tolerance, effector protein specificity, effector protein stability or half-life, effector protein immunogenicity or toxicity. Methods of optimization are discussed in greater detail elsewhere herein.

### Optimization of the Systems

The methods of the present disclosure can involve optimization of selected parameters or variables associated with the composition, system, and/or its functionality, as described herein further elsewhere. Optimization of the composition, system, in the methods as described herein may depend on the target(s), such as the therapeutic target or therapeutic targets, the mode or type of composition, system, modulation, such as composition, system, based therapeutic target(s) modulation, modification, or manipulation, as well as the delivery of the composition, system, components. One or more targets may be selected, depending on the genotypic and/or phenotypic outcome. For instance, one or more therapeutic targets may be selected, depending on (genetic) disease etiology or the desired therapeutic outcome. The (therapeutic) target(s) may be a single gene, locus, or other genomic site, or may be multiple genes, loci or other genomic sites. As is known in the art, a single gene, locus, or other genomic site may be targeted more than once, such as by use of multiple gRNAs.

The activity of the composition and/or system, such as nucleic acid-guided nuclease-based therapy or therapeutics may involve target disruption, such as target mutation, such as leading to gene knockout. The activity of the composition and/or system, such as nucleic acid-guided nuclease-based therapy or therapeutics may involve replacement of particular target sites, such as leading to target correction. nucleic acid-guided nuclease based therapy or therapeutics may involve removal of particular target sites, such as leading to target deletion. The activity of the composition and/or system, such as nucleic acid-guided nuclease-based therapy or therapeutics may involve modulation of target site functionality, such as target site activity or accessibility, leading for instance to (transcriptional and/or epigenetic) gene or genomic region activation or gene or genomic region silencing. The skilled person will understand that modulation of target site functionality may involve nucleic acid-guided nuclease mutation (such as for instance generation of a catalytically inactive nucleic acid-guided nuclease) and/or functionalization (such as for instance fusion of the nucleic acid-guided nuclease with a heterologous functional domain, such as a transcriptional activator or repressor), as described herein elsewhere.

Accordingly, in an aspect, the present disclosure relates to a method as described herein, comprising selection of one or more (therapeutic) target, selecting one or more functionality of the composition and/or system, and optimization of selected parameters or variables associated with the composition and/or its functionality. In a related aspect, the present disclosure relates to a method as described herein, comprising (a) selecting one or more (therapeutic) target loci, (b) selecting one or more composition functionalities, (c) optionally selecting one or more modes of delivery, and preparing, developing, or designing a composition herein selected based on steps (a)-(c).

In certain embodiments, the functionality of the composition and/or system comprises genomic mutation. In certain embodiments, the functionality of the composition and/or system comprises single genomic mutation. In certain embodiments, the functionality of the composition and/or system functionality comprises multiple genomic mutation. In certain embodiments, the functionality of the composition and/or system comprises gene knockout. In certain embodiments, the functionality of the composition and/or system comprises single gene knockout. In certain embodiments, the functionality of the composition and/or system comprises multiple gene knockout. In certain embodiments, the functionality of the composition and/or system comprises gene correction. In certain embodiments, the functionality of the composition and/or system comprises single gene correction. In certain embodiments, the functionality of the composition and/or system comprises multiple gene correction. In certain embodiments, the functionality of the composition and/or system comprises genomic region correction. In certain embodiments, the functionality of the composition and/or system comprises single genomic region correction. In certain embodiments, the functionality of the composition and/or system comprises multiple genomic region correction. In certain embodiments, the functionality of the composition and/or system comprises gene deletion. In certain embodiments, the functionality of the composition and/or system comprises single gene deletion. In certain embodiments, the functionality of the composition and/or system comprises multiple gene deletion. In certain embodiments, the functionality of the composition and/or system comprises genomic region deletion. In certain embodiments, the functionality of the composition and/or system comprises single genomic region deletion. In certain embodiments, the functionality of the composition and/or system comprises multiple genomic region deletion. In certain embodiments, the functionality of the composition and/or system comprises modulation of gene or genomic region functionality. In certain embodiments, the functionality of the composition and/or system comprises modulation of single gene or genomic region functionality. In certain embodiments, the functionality of the composition and/or system comprises modulation of multiple gene or genomic region functionality. In certain embodiments, the functionality of the composition and/or system comprises gene or genomic region functionality, such as gene or genomic region activity. In certain embodiments, the functionality of the composition and/or system comprises single gene or genomic region functionality, such as gene or genomic region activity. In certain embodiments, the functionality of the composition and/or system comprises multiple gene or genomic region functionality, such as gene or genomic region activity. In certain embodiments, the functionality of the composition and/or system comprises modulation gene activity or accessibility optionally leading to transcriptional and/or epigenetic gene or genomic region activation or gene or genomic region silencing. In certain embodiments, the functionality of the composition and/or system comprises modulation single gene activity or accessibility optionally leading to transcriptional and/or epigenetic gene or genomic region activation or gene or genomic region silencing. In certain embodiments, the functionality of the composition and/or system comprises modulation multiple gene activity or accessibility optionally leading to transcriptional and/or epigenetic gene or genomic region activation or gene or genomic region silencing.

Optimization of selected parameters or variables in the methods as described herein may result in optimized or improved the system, such as nucleic acid-guided nuclease-based therapy or therapeutic, specificity, efficacy, and/or safety. In certain embodiments, one or more of the following parameters or variables are taken into account, are selected, or are optimized in the methods of the present disclosure as described herein: nucleic acid-guided nuclease allosteric interactions, nucleic acid-guided nuclease functional domains and functional domain interactions, nucleic acid-guided nuclease specificity, gRNA specificity, composition specificity, PAM restrictiveness, PAM type (natural or modified), PAM nucleotide content, PAM length, nucleic acid-guided nuclease activity, gRNA activity, nucleic acid-guided nuclease/guide complex activity, target cleavage efficiency, target site selection, target sequence length, ability of effector protein to access regions of high chromatin accessibility, degree of uniform enzyme activity across genomic targets, epigenetic tolerance, mismatch/budge tolerance, nucleic acid-guided nuclease stability, nucleic acid-guided nuclease mRNA stability, gRNA stability, nucleic acid-guided nuclease complex stability, nucleic acid-guided nuclease protein or mRNA immunogenicity or toxicity, gRNA immunogenicity or toxicity, nucleic acid-guided nuclease immunogenicity or toxicity, nucleic acid-guided nuclease or mRNA dose or titer, gRNA dose or titer, dose or titer, nucleic acid-guided nuclease protein size, nucleic acid-guided nuclease expression level, gRNA expression level, nucleic acid-guided nuclease expression level, nucleic acid-guided nuclease spatiotemporal expression, gRNA spatiotemporal expression, nucleic acid-guided nuclease/guide molecule spatiotemporal expression.

By means of example, and without limitation, parameter or variable optimization may be achieved as follows. nucleic acid-guided nuclease specificity may be optimized by selecting the most specific nucleic acid-guided nuclease. This may be achieved for instance by selecting the most specific nucleic acid-guided nuclease orthologue or by specific nucleic acid-guided nuclease mutations which increase specificity. gRNA specificity may be optimized by selecting the most specific gRNA. This can be achieved for instance by selecting gRNA having low homology, i.e. at least one or preferably more, such as at least 2, or preferably at least 3, mismatches to off-target sites. The specificity may be optimized by increasing nucleic acid-guided nuclease specificity and/or gRNA specificity as above. PAM restrictiveness may be optimized by selecting a nucleic acid-guided nuclease having to most restrictive PAM recognition. This can be achieved for instance by selecting a nucleic acid-guided nuclease ortholog having more restrictive PAM recognition or by specific nucleic acid-guided nuclease mutations which increase or alter PAM restrictiveness. PAM type may be optimized for instance by selecting the appropriate nucleic acid-guided nuclease, such as the appropriate nucleic acid-guided nuclease recognizing a desired PAM type. The nucleic acid-guided nuclease or PAM type may be naturally occurring or may for instance be optimized based on nucleic acid-guided nuclease mutants having an altered PAM recognition, or PAM recognition repertoire. PAM nucleotide content may for instance be optimized by selecting the appropriate nucleic acid-guided nuclease, such as the appropriate nucleic acid-guided nuclease recognizing a desired PAM nucleotide content. The nucleic acid-guided nuclease or PAM type may be naturally occurring or may for instance be optimized based on nucleic acid-guided nuclease mutants having an altered PAM recognition, or PAM recognition repertoire. PAM length may for instance be optimized by selecting the appropriate nucleic acid-guided nuclease, such as the appropriate nucleic acid-guided nuclease recognizing a desired PAM nucleotide length. The nucleic acid-guided nuclease or PAM type may be naturally occurring or may for instance be optimized based on nucleic acid-guided nuclease mutants having an altered PAM recognition, or PAM recognition repertoire.

Target length or target sequence length may be optimized, for instance, by selecting the appropriate nucleic acid-guided nuclease, such as the appropriate nucleic acid-guided nuclease recognizing a desired target or target sequence nucleotide length. Alternatively, or in addition, the target (sequence) length may be optimized by providing a target having a length deviating from the target (sequence) length typically associated with the nucleic acid-guided nuclease, such as the naturally occurring nucleic acid-guided nuclease. The nucleic acid-guided nuclease or target (sequence) length may be naturally occurring or may for instance be optimized based on nucleic acid-guided nuclease mutants having an altered target (sequence) length recognition, or target (sequence) length recognition repertoire. For instance, increasing or decreasing target (sequence) length may influence target recognition and/or off-target recognition. nucleic acid-guided nuclease activity may be optimized by selecting the most active nucleic acid-guided nuclease. This may be achieved for instance by selecting the most active nucleic acid-guided nuclease ortholog or by specific nucleic acid-guided nuclease mutations which increase activity. The ability of the nucleic acid-guided nuclease protein to access regions of high chromatin accessibility, may be optimized by selecting the appropriate nucleic acid-guided nuclease or mutant thereof, and can consider the size of the nucleic acid-guided nuclease, charge, or other dimensional variables etc. The degree of uniform nucleic acid-guided nuclease activity may be optimized by selecting the appropriate nucleic acid-guided nuclease or mutant thereof, and can consider nucleic acid-guided nuclease specificity and/or activity, PAM specificity, target length, mismatch tolerance, epigenetic tolerance, nucleic acid-guided nuclease and/or gRNA stability and/or half-life, nucleic acid-guided nuclease and/or gRNA immunogenicity and/or toxicity, etc. gRNA activity may be optimized by selecting the most active gRNA. In some embodiments, this can be achieved by increasing gRNA stability through RNA modification. compositions activity may be optimized by increasing nucleic acid-guided nuclease activity and/or gRNA activity as above.

The target site selection may be optimized by selecting the optimal position of the target site within a gene, locus or other genomic region. The target site selection may be optimized by optimizing target location comprises selecting a target sequence with a gene, locus, or other genomic region having low variability. This may be achieved for instance by selecting a target site in an early and/or conserved exon or domain (i.e. having low variability, such as polymorphisms, within a population).

In certain embodiments, optimizing target (sequence) length comprises selecting a target sequence within one or more target loci between 5 and 25 nucleotides. In certain embodiments, a target sequence is 20 nucleotides.

In certain embodiments, optimizing target specificity comprises selecting targets loci that minimize off-target candidates.

In some embodiments, the target site may be selected by minimization of off-target effects (e.g. off-targets qualified as having 1-5, 1-4, or preferably 1-3 mismatches compared to target and/or having one or more PAM mismatches, such as distal PAM mismatches), preferably also considering variability within a population. nucleic acid-guided nuclease stability may be optimized by selecting nucleic acid-guided nuclease having appropriate half-life, such as preferably a short half-life while still capable of maintaining sufficient activity. In some embodiments, this can be achieved by selecting an appropriate nucleic acid-guided nuclease orthologue having a specific half-life or by specific nucleic acid-guided nuclease mutations or modifications which affect half-life or stability, such as inclusion (e.g. fusion) of stabilizing or destabilizing domains or sequences. nucleic acid-guided nuclease mRNA stability may be optimized by increasing or decreasing nucleic acid-guided nuclease mRNA stability. In some embodiments, this can be achieved by increasing or nucleic acid-guided nuclease mRNA stability through mRNA modification. gRNA stability may be optimized by increasing or decreasing gRNA stability. In some embodiments, this can be achieved by increasing or decreasing gRNA stability through RNA modification. The stability may be optimized by increasing or decreasing nucleic acid-guided nuclease stability and/or gRNA stability as above. nucleic acid-guided nuclease protein or mRNA immunogenicity or toxicity may be optimized by decreasing nucleic acid-guided nuclease or mRNA immunogenicity or toxicity. In some embodiments, this can be achieved by mRNA or protein modifications. Similarly, in case of DNA based expression systems, DNA immunogenicity or toxicity may be decreased. gRNA immunogenicity or toxicity may be optimized by decreasing gRNA immunogenicity or toxicity. In some embodiments, this can be achieved by gRNA modifications. Similarly, in case of DNA based expression systems, DNA immunogenicity or toxicity may be decreased. The immunogenicity or toxicity may be optimized by decreasing nucleic acid-guided nuclease immunogenicity or toxicity and/or gRNA immunogenicity or toxicity as above, or by selecting the least immunogenic or toxic nucleic acid-guided nuclease/gRNA combination. Similarly, in case of DNA based expression systems, DNA immunogenicity or toxicity may be decreased. nucleic acid-guided nuclease protein or mRNA dose or titer may be optimized by selecting dosage or titer to minimize toxicity and/or maximize specificity and/or efficacy. gRNA dose or titer may be optimized by selecting dosage or titer to minimize toxicity and/or maximize specificity and/or efficacy. The composition dose or titer may be optimized by selecting dosage or titer to minimize toxicity and/or maximize specificity and/or efficacy. The nucleic acid-guided nuclease size may be optimized by selecting minimal protein size to increase efficiency of delivery, in particular for virus mediated delivery. nucleic acid-guided nuclease, gRNA, or complex thereof expression level may be optimized by limiting (or extending) the duration of expression and/or limiting (or increasing) expression level. This may be achieved for instance by using self-inactivating compositions, systems—such as including a self-targeting (e.g. nucleic acid-guided nuclease targeting) gRNA, by using viral vectors having limited expression duration, by using appropriate promoters for low (or high) expression levels, by combining different delivery methods for individual CRISP-Cas system components, such as virus mediated delivery of nucleic acid-guided nuclease encoding nucleic acid combined with non-virus mediated delivery of gRNA, or virus mediated delivery of gRNA combined with non-virus mediated delivery of nucleic acid-guided nuclease or mRNA. Nucleic acid-guided nuclease, gRNA, or CRISPR-Cas complex spatiotemporal expression may be optimized by appropriate choice of conditional and/or inducible expression systems, including controllable Nucleic acid-guided nuclease activity optionally a destabilized Nucleic acid-guided nuclease and/or a split Nucleic acid-guided nuclease, and/or cell- or tissue-specific expression systems.

In an aspect, the present disclosure relates to a method as described herein, comprising selection of one or more (therapeutic) target, selecting the functionality of the composition and/or system, selecting composition mode of delivery, selecting composition delivery vehicle or expression system, and optimization of selected parameters or variables associated with the composition and/or its functionality, optionally wherein the parameters or variables are one or more selected from nucleic acid-guided nuclease specificity, gRNA specificity, CRISPR-Cas complex specificity, PAM restrictiveness, PAM type (natural or modified), PAM nucleotide content, PAM length, nucleic acid-guided nuclease activity, gRNA activity, nucleic acid-guided nuclease/guide molecule complex activity, target cleavage efficiency, target site selection, target sequence length, ability of effector protein to access regions of high chromatin accessibility, degree of uniform enzyme activity across genomic targets, epigenetic tolerance, mismatch/budge tolerance, nucleic acid-guided nuclease stability, nucleic acid-guided nuclease mRNA stability, gRNA stability, CRISPR-Cas complex stability, nucleic acid-guided nuclease protein or mRNA immunogenicity or toxicity, gRNA immunogenicity or toxicity, nucleic acid-guided nuclease/guide molecule complex immunogenicity or toxicity, nucleic acid-guided nuclease protein or mRNA dose or titer, gRNA dose or titer, CRISPR-Cas complex dose or titer, nucleic acid-guided nuclease protein size, nucleic acid-guided nuclease expression level, gRNA expression level, nucleic acid-guided nuclease/guide molecule complex expression level, nucleic acid-guided nuclease spatiotemporal expression, gRNA spatiotemporal expression, nucleic acid-guided nuclease/guide molecule complex spatiotemporal expression.

It will be understood that the parameters or variables to be optimized as well as the nature of optimization may depend on the (therapeutic) target, the functionality of the composition and/or system, the system mode of delivery, and/or the composition delivery vehicle or expression system.

In an aspect, the present disclosure relates to a method as described herein, comprising optimization of gRNA specificity at the population level. Preferably, said optimization of gRNA specificity comprises minimizing gRNA target site sequence variation across a population and/or minimizing gRNA off-target incidence across a population.

In some embodiments, optimization can result in selection of a nucleic acid-guided nuclease that is naturally occurring or is modified. In some embodiments, optimization can result in selection of a nucleic acid-guided nuclease that has nuclease, nickase, deaminase, transposase, and/or has one or more effector functionalities deactivated or eliminated. In some embodiments, optimizing a PAM specificity can include selecting a nucleic acid-guided nuclease with a modified PAM specificity. In some embodiments, optimizing can include selecting a nucleic acid-guided nuclease having a minimal size. In certain embodiments, optimizing effector protein stability comprises selecting an effector protein having a short half-life while maintaining sufficient activity, such as by selecting an appropriate nucleic acid-guided nuclease orthologue having a specific half-life or stability. In certain embodiments, optimizing immunogenicity or toxicity comprises minimizing effector protein immunogenicity or toxicity by protein modifications. In certain embodiments, optimizing functional specific comprises selecting a protein effector with reduced tolerance of mismatches and/or bulges between the guide RNA and one or more target loci.

In certain embodiments, optimizing efficacy comprises optimizing overall efficiency, epigenetic tolerance, or both. In certain embodiments, maximizing overall efficiency comprises selecting an effector protein with uniform enzyme activity across target loci with varying chromatin complexity, selecting an effector protein with enzyme activity limited to areas of open chromatin accessibility. In certain embodiments, chromatin accessibility is measured using one or more of ATAC-seq, or a DNA-proximity ligation assay. In certain embodiments, optimizing epigenetic tolerance comprises optimizing methylation tolerance, epigenetic mark competition, or both. In certain embodiments, optimizing methylation tolerance comprises selecting an effector protein that modify methylated DNA. In certain embodiments, optimizing epigenetic tolerance comprises selecting an effector protein unable to modify silenced regions of a chromosome, selecting an effector protein able to modify silenced regions of a chromosome, or selecting target loci not enriched for epigenetic markers

In certain embodiments, selecting an optimized guide RNA comprises optimizing gRNA stability, gRNA immunogenicity, or both, or other gRNA associated parameters or variables as described herein elsewhere.

In certain embodiments, optimizing gRNA stability and/or gRNA immunogenicity comprises RNA modification, or other gRNA associated parameters or variables as described herein elsewhere. In certain embodiments, the modification comprises removing 1-3 nucleotides form the 3′ end of a target complementarity region of the gRNA. In certain embodiments, modification comprises an extended gRNA and/or trans RNA/DNA element that create stable structures in the gRNA that compete with gRNA base pairing at a target of off-target loci, or extended complimentary nucleotides between the gRNA and target sequence, or both.

In certain embodiments, the mode of delivery comprises delivering gRNA and/or nucleic acid-guided nuclease, delivering gRNA and/or nucleic acid-guided nuclease mRNA, or delivery gRNA and/or nucleic acid-guided nuclease as a DNA based expression system. In certain embodiments, the mode of delivery further comprises selecting a delivery vehicle and/or expression systems from the group consisting of liposomes, lipid particles, nanoparticles, biolistics, or viral-based expression/delivery systems. In certain embodiments, expression is spatiotemporal expression is optimized by choice of conditional and/or inducible expression systems, including controllable nucleic acid-guided nuclease activity optionally a destabilized nucleic acid-guided nuclease and/or a split nucleic acid-guided nuclease, and/or cell- or tissue-specific expression system.

The methods as described herein may further involve selection of the mode of delivery. In certain embodiments, gRNA (and tracr, if and where needed, optionally provided as a sgRNA) and/or nucleic acid-guided nuclease are or are to be delivered. In certain embodiments, gRNA (and tracr, if and where needed, optionally provided as a sgRNA) and/or nucleic acid-guided nuclease mRNA are or are to be delivered. In certain embodiments, gRNA (and tracr, if and where needed, optionally provided as a sgRNA) and/or nucleic acid-guided nuclease provided in a DNA-based expression system are or are to be delivered. In certain embodiments, delivery of the individual system components comprises a combination of the above modes of delivery. In certain embodiments, delivery comprises delivering gRNA and/or nucleic acid-guided nuclease protein, delivering gRNA and/or nucleic acid-guided nuclease mRNA, or delivering gRNA and/or nucleic acid-guided nuclease as a DNA based expression system.

The methods as described herein may further involve selection of the composition delivery vehicle and/or expression system. Delivery vehicles and expression systems are described herein elsewhere. By means of example, delivery vehicles of nucleic acids and/or proteins include nanoparticles, liposomes, etc. Delivery vehicles for DNA, such as DNA-based expression systems include for instance biolistics, viral based vector systems (e.g. adenoviral, AAV, lentiviral), etc. the skilled person will understand that selection of the mode of delivery, as well as delivery vehicle or expression system may depend on for instance the cell or tissues to be targeted. In certain embodiments, the delivery vehicle and/or expression system for delivering the compositions, systems, or components thereof comprises liposomes, lipid particles, nanoparticles, biolistics, or viral-based expression/delivery systems.

### Considerations for Therapeutic Applications

A consideration in genome editing therapy is the choice of sequence-specific nuclease, such as a variant of a nucleic acid-guided nuclease. Each nuclease variant may possess its own unique set of strengths and weaknesses, many of which must be balanced in the context of treatment to maximize therapeutic benefit. For a specific editing therapy to be efficacious, a sufficiently high level of modification must be achieved in target cell populations to reverse disease symptoms. This therapeutic modification ‘threshold’ is determined by the fitness of edited cells following treatment and the amount of gene product necessary to reverse symptoms. With regard to fitness, editing creates three potential outcomes for treated cells relative to their unedited counterparts: increased, neutral, or decreased fitness. In the case of increased fitness, corrected cells may be able and expand relative to their diseased counterparts to mediate therapy. In this case, where edited cells possess a selective advantage, even low numbers of edited cells can be amplified through expansion, providing a therapeutic benefit to the patient. Where the edited cells possess no change in fitness, an increase the therapeutic modification threshold can be warranted. As such, significantly greater levels of editing may be needed to treat diseases, where editing creates a neutral fitness advantage, relative to diseases where editing creates increased fitness for target cells. If editing imposes a fitness disadvantage, as would be the case for restoring function to a tumor suppressor gene in cancer cells, modified cells would be outcompeted by their diseased counterparts, causing the benefit of treatment to be low relative to editing rates. This may be overcome with supplemental therapies to increase the potency and/or fitness of the edited cells relative to the diseased counterparts.

In addition to cell fitness, the amount of gene product necessary to treat disease can also influence the minimal level of therapeutic genome editing that can treat or prevent a disease or a symptom thereof. In cases where a small change in the gene product levels can result in significant changes in clinical outcome, the minimal level of therapeutic genome editing is less relative to cases where a larger change in the gene product levels are needed to gain a clinically relevant response. In some embodiments, the minimal level of therapeutic genome editing can range from 0.1 to 1%, 1-5%, 5-10%, 10-15%, 15-20%, 20-25%, 25-30%, 30-35%, 35-40%, 40-45%. 45-50%, or 50-55%. Thus, where a small change in gene product levels can influence clinical outcomes and diseases where there is a fitness advantage for edited cells, are ideal targets for genome editing therapy, as the therapeutic modification threshold is low enough to permit a high chance of success.

The activity of NHEJ and HDR DSB repair can vary by cell type and cell state. NHEJ is not highly regulated by the cell cycle and is efficient across cell types, allowing for high levels of gene disruption in accessible target cell populations. In contrast, HDR acts primarily during S/G2 phase, and is therefore restricted to cells that are actively dividing, limiting treatments that require precise genome modifications to mitotic cells [Ciccia, A. & Elledge, S. J. Molecular cell 40, 179-204 (2010); Chapman, J. R., et al. Molecular cell 47, 497-510 (2012)].

The efficiency of correction via HDR may be controlled by the epigenetic state or sequence of the targeted locus, or the specific repair template configuration (single vs. double stranded, long vs. short homology arms) used [Hacein-Bey-Abina, S., et al. The New England journal of medicine 346, 1185-1193 (2002); Gaspar, H. B., et al. Lancet 364, 2181-2187 (2004); Beumer, K. J., et al. G3 (2013)]. The relative activity of NHEJ and HDR machineries in target cells may also affect gene correction efficiency, as these pathways may compete to resolve DSBs [Beumer, K. J., et al. Proceedings of the National Academy of Sciences of the United States of America 105, 19821-19826 (2008)]. HDR also imposes a delivery challenge not seen with NHEJ strategies, as it uses the concurrent delivery of nucleases and repair templates. Thus, these differences can be kept in mind when designing, optimizing, and/or selecting a nucleic acid-guided nuclease based therapeutic as described in greater detail elsewhere herein.

nucleic acid-guided nuclease-based polynucleotide modification application can include combinations of proteins, small RNA molecules, and/or repair templates, and can make, in some embodiments, delivery of these multiple parts substantially more challenging than, for example, traditional small molecule therapeutics. Two main strategies for delivery of compositions, systems, and components thereof have been developed: ex vivo and in vivo. In some embodiments of ex vivo treatments, diseased cells are removed from a subject, edited and then transplanted back into the patient. In other embodiments, cells from a healthy allogeneic donor are collected, modified using a composition or component thereof, to impart various functionalities and/or reduce immunogenicity, and administered to an allogeneic recipient in need of treatment. Ex vivo editing has the advantage of allowing the target cell population to be well defined and the specific dosage of therapeutic molecules delivered to cells to be specified. The latter consideration may be particularly important when off-target modifications are a concern, as titrating the amount of nuclease may decrease such mutations (Hsu et al., 2013). Another advantage of ex vivo approaches is the typically high editing rates that can be achieved, due to the development of efficient delivery systems for proteins and nucleic acids into cells in culture for research and gene therapy applications.

In vivo polynucleotide modification via compositions, systems, and/or components thereof involves direct delivery of the compositions, systems, and/or components thereof to cell types in their native tissues. In vivo polynucleotide modification via compositions, systems, and/or components thereof allows diseases in which the affected cell population is not amenable to ex vivo manipulation to be treated. Furthermore, delivering compositions, systems, and/or components thereof to cells in situ allows for the treatment of multiple tissue and cell types.

In some embodiments, such as those where viral vector systems are used to generate viral particles to deliver the composition and/or component thereof to a cell, the total cargo size of the composition and/or component thereof should be considered as vector systems can have limits on the size of a polynucleotide that can be expressed therefrom and/or packaged into cargo inside of a viral particle. In some embodiments, the tropism of a vector system, such as a viral vector system, should be considered as it can impact the cell type to which the composition or component thereof can be efficiently and/or effectively delivered.

When delivering a system or component thereof via a viral-based system, it can be important to consider the amount of viral particles that will be needed to achieve a therapeutic effect so as to account for the potential immune response that can be elicited by the viral particles when delivered to a subject or cell(s). When delivering a system or component thereof via a viral based system, it can be important to consider mechanisms of controlling the distribution and/or dosage of the system in vivo. Generally, to reduce the potential for off-target effects, it is optimal but not necessarily required, that the amount of the system be as close to the minimum or least effective dose. In practice this can be challenging to do.

In some embodiments, it can be important to consider the immunogenicity of the system or component thereof. In embodiments, where the immunogenicity of the system or component thereof is of concern, the immunogenicity system or component thereof can be reduced. By way of example only, the immunogenicity of the system or component thereof can be reduced using the approach set out in Tangri et al. Accordingly, directed evolution or rational design may be used to reduce the immunogenicity of the nucleic acid-guided nuclease in the host species (human or other species).

### Xenotransplantation

The present disclosure also contemplates use of the composition described herein, e.g. nucleic acid-guided nuclease protein systems, to provide RNA-guided DNA nucleases adapted to be used to provide modified tissues for transplantation. For example, RNA-guided DNA nucleases may be used to knockout, knockdown or disrupt selected genes in an animal, such as a transgenic pig (such as the human heme oxygenase-1 transgenic pig line), for example by disrupting expression of genes that encode epitopes recognized by the human immune system, i.e. xenoantigen genes. Candidate porcine genes for disruption may for example include α(1,3)-galactosyltransferase and cytidine monophosphate-N-acetylneuraminic acid hydroxylase genes (see PCT Patent Publication WO 2014/066505). In addition, genes encoding endogenous retroviruses may be disrupted, for example the genes encoding all porcine endogenous retroviruses (see Yang et al., 2015, Genome-wide inactivation of porcine endogenous retroviruses (PERVs), Science 27 Nov. 2015: Vol. 350 no. 6264 pp. 1101-1104). In addition, RNA-guided DNA nucleases may be used to target a site for integration of additional genes in xenotransplant donor animals, such as a human CD55 gene to improve protection against hyperacute rejection.

Embodiments of the present disclosure also relate to methods and compositions related to knocking out genes, amplifying genes and repairing particular mutations associated with DNA repeat instability and neurological disorders (Robert D. Wells, Tetsuo Ashizawa, Genetic Instabilities and Neurological Diseases, Second Edition, Academic Press, Oct. 13, 2011—Medical). Specific aspects of tandem repeat sequences have been found to be responsible for more than twenty human diseases (New insights into repeat instability: role of RNA•DNA hybrids. Mclvor E I, Polak U, Napierala M. RNA Biol. 2010 September-October; 7(5):551-8). The present effector protein systems may be harnessed to correct these defects of genomic instability.

Several further aspects of the present disclosure relate to correcting defects associated with a wide range of genetic diseases which are further described on the website of the National Institutes of Health under the topic subsection Genetic Disorders (website at health.nih.gov/topic/GeneticDisorders). The genetic brain diseases may include but are not limited to Adrenoleukodystrophy, Agenesis of the Corpus Callosum, Aicardi Syndrome, Alpers' Disease, Alzheimer's Disease, Barth Syndrome, Batten Disease, CADASIL, Cerebellar Degeneration, Fabry's Disease, Gerstmann-Straussler-Scheinker Disease, Huntington's Disease and other Triplet Repeat Disorders, Leigh's Disease, Lesch-Nyhan Syndrome, Menkes Disease, Mitochondrial Myopathies and NINDS Colpocephaly. These diseases are further described on the website of the National Institutes of Health under the subsection Genetic Brain Disorders.

### Kits

In one aspect, the present disclosure provides kits containing any one or more of the elements disclosed in the above methods and compositions. In some embodiments, the kit comprises a vector system as taught herein or one or more of the components of the CRISPR-Cas system or complex as taught herein, such as crRNAs and/or Cas protein or Cas protein encoding mRNA, and instructions for using the kit. Elements may be provided individually or in combinations, and may be provided in any suitable container, such as a vial, a bottle, or a tube. In some embodiments, the kit includes instructions in one or more languages, for example in more than one language. The instructions may be specific to the applications and methods described herein. In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g., in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide sequence for insertion into a vector so as to operably link the guide or crRNA sequence and a regulatory element. In some embodiments, the kit comprises a homologous recombination template polynucleotide. In some embodiments, the kit comprises one or more of the vectors and/or one or more of the polynucleotides described herein. The kit may advantageously allow to provide all elements of the systems of the present disclosure.

The invention is further described in the following examples, which do not limit the scope of the invention described in the claims.

The present application also provides aspects and embodiments as set forth in the following numbered Statements:

Statement 1. A non-naturally occurring or engineered composition comprising: a Cas protein that comprises a RuvC domain and a HNH domain, and is less than 850 amino acids in size; and a guide sequence capable of forming a complex with the Cas protein and directing the complex to bind to a target sequence.

Statement 2. The composition of Statement 1, wherein the Cas protein is a Type II Cas protein.

Statement 3. The composition of any one of the proceeding Statements, wherein the Type II Cas protein is a Type II-B Cas protein.

Statement 4. The composition of any one of the proceeding Statements, wherein the Type II Cas protein is a Type II-C Cas protein.

Statement 5. The composition of any one of the proceeding Statements, wherein the Type II Cas protein is Cas9 or an ortholog thereof.

Statement 6. The composition of any one of the proceeding Statements, wherein the Cas protein is a protein from Table 12.

Statement 7. The composition of any one of the proceeding Statements, wherein the Cas protein is derived from Gammaproteobacteria bacterium AqS3, Deltaproteobacteria bacterium GWF2_42_12, JGI Metagenome: IMG 3300025323, Nitrospirae bacterium RBG_13_39_12, or Nitrospiraceae bacterium isolate UBA9935.

Statement 8. The composition of any one of the proceeding Statements, wherein the composition comprises two or more guide sequences capable of hybridizing to two different target sequences or different regions of a target sequence.

Statement 9. The composition of any one of the proceeding Statements, wherein the guide sequence is capable of hybridizing to one or more target sequences in a prokaryotic cell.

Statement 10. The composition of any one of the proceeding Statements, wherein the guide sequence is capable of hybridizing to one or more target sequences in a eukaryotic cell.

Statement 11. The composition of any one of the proceeding Statements, wherein the Cas protein comprises one or more nuclear localization signals.

Statement 12. The composition of any one of the proceeding Statements, wherein the Cas protein comprises two or more nuclear localization signals.

Statement 13. The composition of any one of the proceeding Statements, wherein the Cas protein comprises one or more nuclear export signals.

Statement 14. The composition of any one of the proceeding Statements, wherein the Cas protein is catalytically inactive.

Statement 15. The composition of any one of the proceeding Statements, wherein the Cas protein is a nickase.

Statement 16. The composition of any one of the proceeding Statements, wherein the Cas protein is associated with one or more functional domains.

Statement 17. The composition of Statement 16, wherein the one or more functional domains comprises one or more heterologous functional domains.

Statement 18. The composition of Statement 16 or 17, wherein the one or more functional domains cleaves the target sequence.

Statement 19. The composition of any one of Statements 16-18, wherein the one or more functional domains modifies transcription or translation of the target sequence.

Statement 20. The composition of any one of Statements 16-19, wherein the one or more functional domains comprises one or more transcriptional activation domains.

Statement 21. The composition of Statement 20, wherein the one or more transcriptional activation domains comprises VP64.

Statement 22. The composition of any one of Statements 16-21, wherein the one or more functional domains comprises one or more transcriptional repression domains.

Statement 23. The composition of Statement 22, wherein the one or more transcriptional repression domains comprises a KRAB domain or a SID domain.

Statement 24. The composition of any one of Statements 16-23, wherein the one or more functional domains comprises one or more nuclease domains.

Statement 25. The composition of Statement 24, wherein the one or more nuclease domains comprises Fok1.

Statement 26. The composition of any one of Statements 16-25, wherein the one or more functional domains has one or more of the following activities: methylase activity, demethylase activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity and nucleic acid binding activity.

Statement 27. The composition of any one of the proceeding Statements, further comprising a recombination template.

Statement 28. The composition of Statement 27, wherein the recombination template is inserted by homology-directed repair (HDR).

Statement 29. The composition of any one of the proceeding Statements, further comprising a tracr RNA.

Statement 30. The composition of any one of the proceeding Statements, wherein the Cas protein is a chimeric protein comprising a first fragment from a first Cas protein and a second fragment from a second Cas protein.

Statement 31. The composition of any one of the proceeding Statements, further comprising a nucleotide deaminase or a catalytic domain thereof.

Statement 32. The composition of Statement 31, wherein the nucleotide deaminase is an adenosine deaminase.

Statement 33. The composition of any one of Statements 31-32, wherein the nucleotide deaminase is a cytidine deaminase.

Statement 34. The composition of any one of Statements 31-33, wherein the nucleotide deaminase or catalytic domain thereof is covalently or non-covalently linked to the Cas protein or the guide sequence, or is adapted to link thereof after delivered to a cell.

Statement 35. The composition of any one of Statements 31-34, wherein the nucleotide deaminase or catalytic domain thereof has been modified to increase its activity against a DNA-RNA heteroduplex.

Statement 36. The composition of any one of Statements 31-35, wherein the nucleotide deaminase or catalytic domain thereof has been modified to reduce off-target effects.

Statement 37. The composition of any one of Statements 31-36, wherein the composition is capable of modifying one or more nucleotides in the target sequence.

Statement 38. The composition of Statement 37, wherein modification of the one or more nucleotides in the target sequence remedies a disease caused by a G→A or C→T point mutation or a pathogenic SNP.

Statement 39. The composition of Statement 37 or 38, wherein the disease is cancer, haemophilia, beta-thalassemia, Marfan syndrome, or Wiskott-Aldrich syndrome.

Statement 40. The composition of any one of Statements 37-39, wherein modification of the one or more nucleotides in the target sequence remedies a disease caused by a T→C or A→G point mutation or a pathogenic SNP.

Statement 41. The composition of any one of Statements 37-40, wherein modification of the one or more nucleotides at the target sequence inactivates a gene.

Statement 42. The composition of any one of Statements 37-41, wherein modification of the one or more nucleotides modifies gene product encoded at the target sequence or expression of the gene product.

Statement 43. The composition of any one of the proceeding Statements, further comprising a reverse transcriptase or functional fragment thereof.

Statement A non-naturally occurring or engineered composition comprising one or more polynucleotide sequences encoding: a Cas protein that comprises a RuvC domain and a HNH domain, and is less than 900 amino acids in size; and a guide sequence capable of forming a complex with the Cas protein and directing the complex to bind to a target sequence.

Statement 45. The composition of Statement 44, wherein the one or more polynucleotide sequences are codon optimized to express in a eukaryote.

Statement 46. The composition of any one of Statements 44-45, wherein the one or more polynucleotide sequences is mRNA.

Statement 47. The composition of any one of Statements 44-46, wherein the one or more polynucleotide sequences further encode a reverse transcriptase or functional fragment thereof.

Statement 48. A vector system comprising the one or more polynucleotides sequences of any one of Statements 44 to 46.

Statement 49. The vector system of Statement 48, comprising: a first regulatory element operably linked to the polynucleotide sequence encoding the Cas protein; and a second regulatory element operably linked to the polynucleotide sequence encoding the guide sequence.

Statement 50. The vector system of Statement 49, wherein the first and/or second regulatory element is a promoter.

Statement 51. The vector system of Statement 50, wherein the promoter is a minimal promoter.

Statement 52. The vector system of Statement 51, wherein the minimal promoter is Mecp2 promoter, tRNA promoter, or U6 promoter.

Statement 53. The vector system of any one of Statements 48-52, which is comprised in a single vector.

Statement 54. The vector system of any one of Statements 48-52, wherein the one or more vectors comprises viral vectors.

Statement 55. The vector system of any one of Statements 48-52, wherein the one or more vectors comprises retroviral, lentiviral, adenoviral, adeno-associated, or Herpes simplex viral vectors.

Statement 56. A delivery system comprising the system of any one of Statements 1 to 55 and a delivery vehicle.

Statement 57. The delivery system of Statement 56, wherein the delivery vehicle comprises lipids, sugars, metals, proteins, liposomes, nanoparticles, exosomes, microvesicles, nucleic acid nanoassemblies, a gene gun, an implantable device, or a vector system.

Statement 58. The delivery system of Statement 56 or 57, wherein the delivery vehicle comprises ribonucleoproteins.

Statement 59. A cell comprising the composition of any one of Statements 1 to 55.

Statement 60. The cell of Statement 59, wherein the cell is a eukaryotic cell, a human or non-human animal cell, a therapeutic T cell, antibody-producing B-cell, a stem cell, or a plant cell.

Statement 61. A tissue, organ, or organism comprising the cell of Statement 59.

Statement 62. A cell product from the cell of Statement 59.

Statement 63. A method of modifying one or more target sequences, the method comprising contacting the one or more target sequences with a composition of any one of Statements 1 to 55.

Statement 64. The method of Statement 63, wherein the composition further comprises a recombination template, and wherein modifying the one or more target sequences comprises insertion of the recombination template or a portion thereof.

Statement 65. The method of Statement 63 or 64, wherein the one or more target sequences is in a prokaryotic cell.

Statement 66. The method of any one of Statements 63-65, wherein the one or more target sequences is in a eukaryotic cell.

Statement 67. The method of any one of Statements 63-67, wherein the one or more target sequences is comprised in a nucleic acid molecule in vitro.

Statement 68. A cell obtained from the method of any one of Statements 63-67.

Statement 69. The cell or progeny thereof of Statement 68, wherein the cell is a eukaryotic cell, a human or non-human animal cell, a therapeutic T cell, antibody-producing B-cell, a stem cell, or a plant cell.

Statement 70. A non-human animal or plant comprising the modified cell or progeny thereof of Statement 68 or 69.

Statement 71. A modified cell or progeny thereof of Statement 68 or 69 for use in therapy.

Statement 72. A method of treating a disease, disorder, or infection comprising administering an effective amount of the composition of any one of Statements 1 to 55 in a subject in need thereof.

Statement 73. A method of producing a plant having a modified trait of interest encoded by a gene of interest, the method comprises contacting a plant cell with a composition of any one of Statements 1 to 55, thereby either modifying or introducing the gene of interest, and regenerating a plant from the plant cell.

Statement 74. A method of identifying a trait of interest in a plant, the trait of interest encoded by a gene of interest, the method comprises contacting a plant cell with a composition any one of Statements 1 to 55, thereby identifying the gene of interest.

### EXAMPLES

### Example 1—Exemplary Small Cas9 Proteins

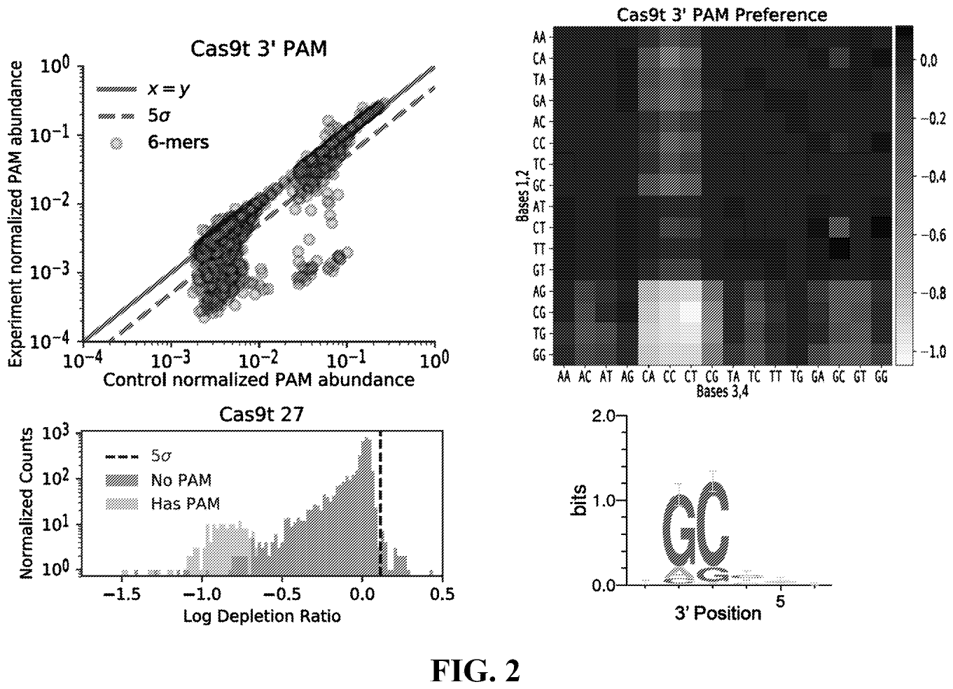

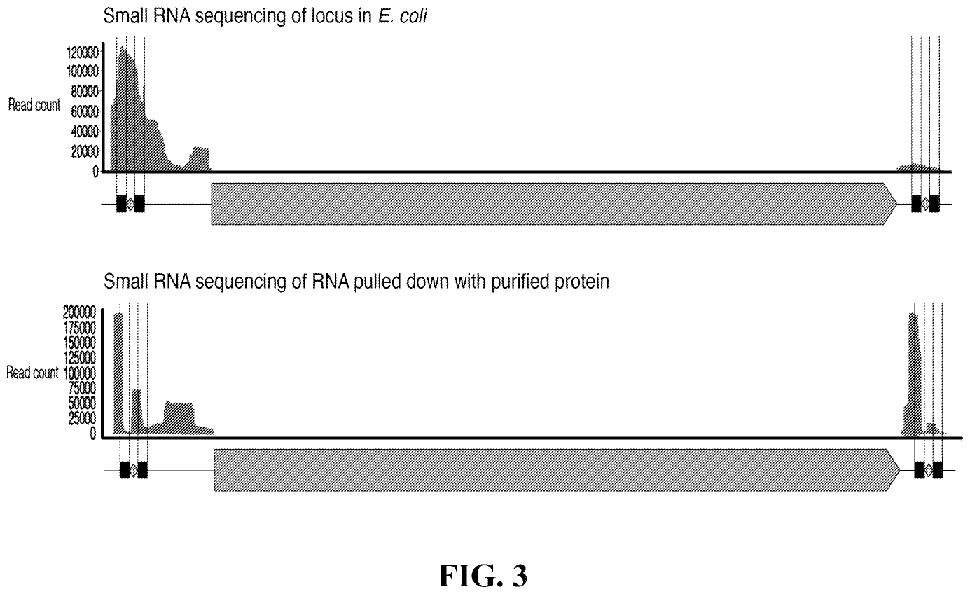

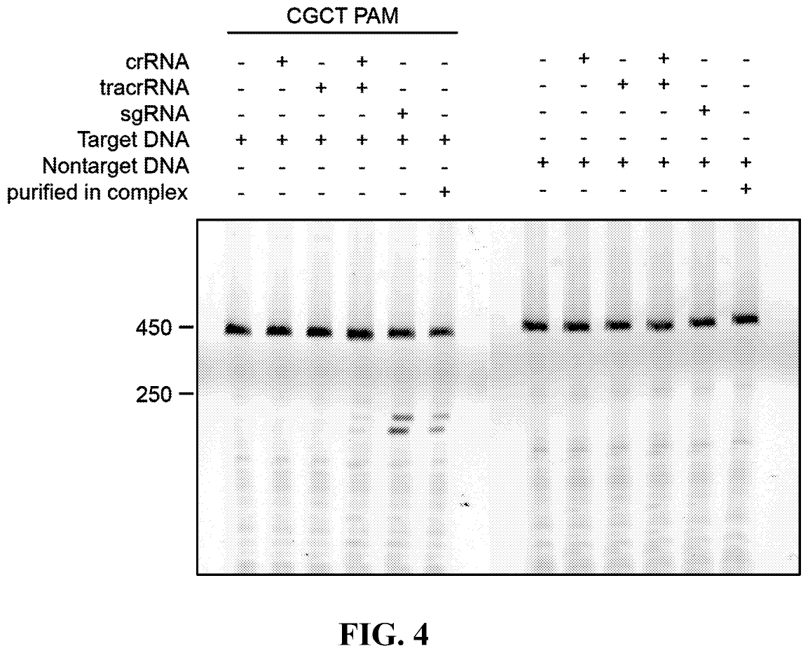

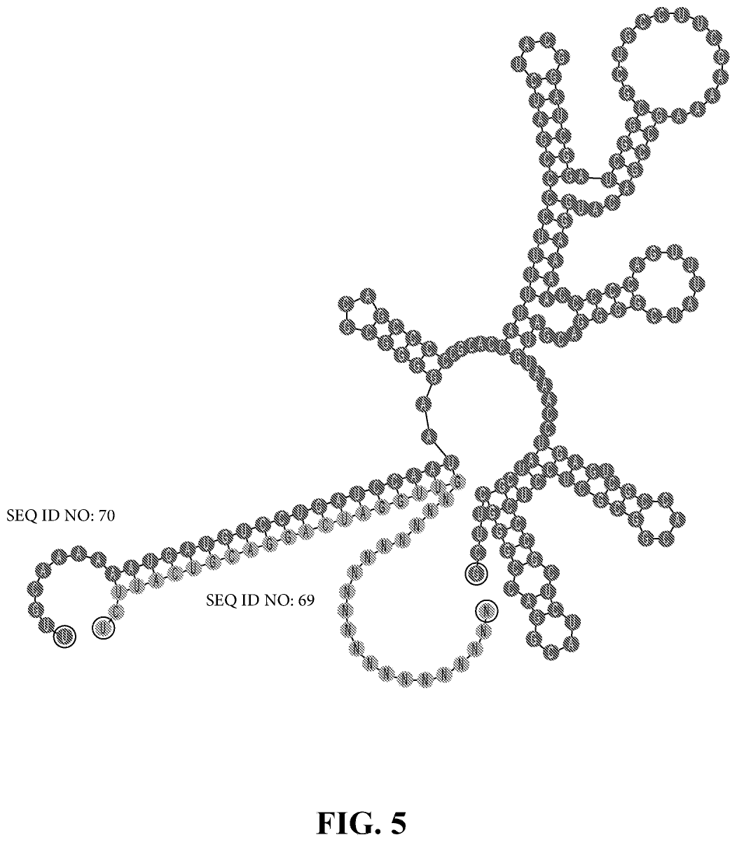

An exemplary small Type II-C Cas9 (Cas9-t) protein is shown in FIG. 1. The exemplary Cas9-t had an NGCH PAM (FIG. 2). The purification pulldown showed more distinct associated small RNAs (FIG. 3). The purified Cas9-t was able to cleave DNA in vitro at 37° C. with a single guide RNA (FIG. 4). FIG. 5 shows the structure of the crRNA and tracrRNA in the form of a complex.

The relevant sequences of the exemplary of the Type II-C Cas9 is shown in Table 10 below.

TABLE 10

| | |
|---|---|
| Cas9t-1 | 1 ctaccccgca aggaaggagc cgccgaccga tgtcttgtta ggaaaactaa tccttcggaa |
| Contig | 61 attagtcgag aaggggctcg cccccccaaa agccaacctg tcgtggcaat gcatcgaaag |
| | 121 cctgaccatc accccgtgga tatgggatcg cttcgccgag cacgggatcg aagtcgcgct |
| | 181 cggcgagatg gtcaggcggc ggggcgaaga gaaatcaaaa aaaaatcgaa cggggggttg |
| | 241 acttttggat aatagccttc ataatctcgc ccatcgatag ccgagcaacg aagttggatc |
| | 301 aggacgtcat tcttcgaatt cgaaaacgcg gggattgctc gaatgggtgg atgatatgtt |
| | 361 ggatcaggac gtcacttttc gaattcgaaa acgcaagtcg aagttggcga cttgatcaaa |
| | 421 tagttggatc aggacgtcac ttttcgaatt cgaaaactaa agaccgctcg gctgatactt |
| | 481 gacggaagtt ggatcaggac gtcacttttc gaattcgaaa accgatctcg cctaagtgat |
| | 541 tgattcagcg aagaaattgt agaatcgcag ttgcaaaaaa tgatgtcctg atccaataag |
| | 601 gggcgcagcc cccgcaccat ttttccccga tctacggatc ggatcggcgc tgccttcgaa |
| | 661 aagccgacat ggaaaacccc cagtttatcg gggacgatgt aaacctgagt cgccacggcg |
| | 721 ctcatctcgc ccgctctacg gaccgggctt cgctggaaac tgggagcggt aaccatgaaa |
| | 781 caactcacac tcgatgtcgg catcgcttcg atcggctggg cgattgtcag taaaaaaggg |
| | 841 aacgtcaaag ccggatcacg catctttccc gacgccaaat ctgggcggga aagcaaccaa |
| | 901 tcccgacggg cggctcggct gatgcgccga ggatatcggc gcaaagcaaa gcgccgagcc |
| | 961 gacacgctcg ccatcattcg ctccatccac cccggcttcg accccgaagg gcaccctgac |
| | 1021 atcgagcgcg aagctctgat caaagccatc attacccccg gcgcccccac cccatcgctc |
| | 1081 gaccaactcg cttgcgcgtt ccagcgattc gccaagtctc ggtggccgca atattcgaga |
| | 1141 actctcccaa aacgaaccga gcgggaagac cagttcatcc aagtttgggt gatcgccgag |
| | 1201 cgaacctatc ccgaccgctt tacccccgac gtcgcaaccc gcttgcttca ggcgatattc |
| | 1261 tttcagcgcc ctatcaaaga cggcgaccgc gcgaaatgtc aactattcag gcatcacggt |
| | 1321 gacaaagccc cgctcgtcgg ctggacgcac gaacccgaac tccaacgatt cgccatcttg |
| | 1381 tccgatctgt ccaatctcac tatcgggatc ggctcgactg ataacctgct ttgcgaatat |
| | 1441 cccgacatca tcgaagactt ggagacccga tgcttcgaga ccggcatgag ctggagagag |
| | 1501 atagccgaac acgtcaagga agtcatcggc aaaggggtgg tgtttcgagg cattgacggg |
| | 1561 cagaaaaaag tcgggcggaa cgggatcggg cctgccaaac tcgaaacaat cgacgaagaa |
| | 1621 ggcaactcta ccaagagcac cgcttcgatg tcggtcgaag cggcggtaat gatctatcac |
| | 1681 caaatgaaag cggatcgatg ccgagcggca accgctaaaa aaacgctgat cgacgcaggg |
| | 1741 gctctttcag cccccctaac cgccaaagat atcaaacgtg gcgatcgcac tctcaccatc |
| | 1801 accgaactga tggatatggc gggcaggatt accgacccga ctatcagggc gatctaccac |
| | 1861 caagtcgaga tgttggtcaa cgagttgatc gcccgcttcg gtaaacccga acgcatcgtt |
| | 1921 atcgaagccc aaaaggaaat cgggcgaagc atcgaagaca tcgagaaagc gatggcaaga |
| | 1981 gagcgagaaa agcatatcga acggcaacga gagaatcgag cccgcaacgc cgcaatgggt |
| | 2041 accaaagccc gctttgcccg cctgtgcgcc atcaggggtg atcgatgctt tatcagcgac |
| | 2101 cgacccgcag ccgaagtcgg ccacctgatc gccgattcga tcgggggcac gcttgaaatg |
| | 2161 gctaacctga tcccgatcga ccctgccatc aataaagaga tgggcaaccg cactccctac |
| | 2221 gaagcctttc gaaagactga gtattggtcg atcatccagc gcaaactgca agcgcttgaa |
| | 2281 gacgaagtga aggctttgaa accgccgaaa gggacaaaag gaacggcgtg gacgatctat |
| | 2341 caccgagcca agcatcagtt tgattttttc gcttggcgat ttcaatcgaa tgcgagagaa |
| | 2401 acccatcaaa ggaactttcg ccccggctcg cttgacgacc tgcggtggat cgaaaacttg |
| | 2461 ctctttctcg gcgttgcccc gatttgcgac aatatccgaa tcgtcagtgg gcgaacaacc |
| | 2521 gagcgcattc gccgagagat acttggaatg gacaaagacc gccgagacca tcgccaccat |
| | 2581 gcgctcgatg cgcttgctat catgctcgcc aatcctttga agccgtggga tttgaaatcg |
| | 2641 agcaattcgc tcggtatccc gctcggccga atcaaacaag ccttcgccga cgctgtcgtc |
| | 2701 tcgcaaaagc aagaccactc gcttcgcact gcgttgcata aagagaacgc gatcccgaag |
| | 2761 accaagcggg gggctgcata tcgaaaaatc ggaacggggg cgagcgaacg cgtagtcgac |
| | 2821 acccaatcaa aggcctattg cgaagtttgg gcgttgccta acgggaagtg ggaagcggtc |
| | 2881 gtagtgtcga gtttcgacgc tgcgcaaaag aactaccggc aagggattga ccatcgcccc |
| | 2941 catcccgccg ctcgactggt tatgcgcttg ttcaaatccg atttgctcgg tatcgggggc |
| | 3001 aagatatacc gggttcagga actactcggt tctggctcga tctacttggt cgatcatcga |
| | 3061 ttcgctggca ctattcgaga cgcccgcgca gtttgtaaga cgggcgtcaa tgtcgatttc |
| | 3121 tttagcaaag ggggtgattc attgcgcaag gcgggggctc gtcttgtttc gattcgtaag |
| | 3181 agttgggtgg gatcgtgagt cacttttcga attcggaaac ttttccgacc gacaagtact |
| | 3241 cgatcatcta gttggatcag gacgtcatgc ttcgaattcg aaaacgtcag tcgggtgacc |
| | 3301 tacgagcgat agggggttgg atcaggacgt cacttttcga attcgaaaac cattcgatgc |
| | 3361 cgatgcctac accgcaaaga gttggatcag gacgtcatgc ttcgaattcg aaaaccttcg |
| | 3421 tttcaataac c (SEQ ID NO: 34) |
| Cas9t-1 | CTGGGAGCGGTAACCATGAAACAACTCACACTCGATGTCGGCATCGCTTCGATCGGCTGGGCGATTGTCAGTA |
| Contig | AAAAAGGGAACGTCAAAGCCGGATCACGCATCTTTCCCGACGCCAAATCTGGGCGGGAAAGCAACCAATCCC |
| for | GACGGGCGGCTCGGCTGATGCGCCGAGGATATCGGCGCAAAGCAAAGCGCCGAGCCGACACGCTCGCCATCA |
| synthesis | TTCGCTCCATCCACCCCGGCTTCGACCCCGAAGGGCACCCTGACATCGAGCGCGAAGCTCTGATCAAAGCCAT |
| | CATTACCCCCGGCGCCCCCACCCCATCGCTCGACCAACTCGCTTGCGCGTTCCAGCGATTCGCCAAGTCTCGGT |
| | GGCCGCAATATTCGAGAACTCTCCCAAAACGAACCGAGCGGGAAGACCAGTTCATCCAAGTTTGGGTGATCG |
| | CCGAGCGAACCTATCCCGACCGCTTTACCCCCGACGTCGCAACCCGCTTGCTTCAGGCGATATTCTTTCAGCGC |
| | CCTATCAAAGACGGCGACCGCGCGAAATGTCAACTATTCAGGCATCACGGTGACAAAGCCCCGCTCGTCGGCT |
| | GGACGCACGAACCCGAACTCCAACGATTCGCCATCTTGTCCGATCTGTCCAATCTCACTATCGGGATCGGCTC |
| | GACTGATAACCTGCTTTGCGAATATCCCGACATCATCGAAGACTTGGAGACCCGATGCTTCGAGACCGGCATG |
| | AGCTGGAGAGAGATAGCCGAACACGTCAAGGAAGTCATCGGCAAAGGGGTGGTGTTTCGAGGCATTGACGGG |
| | CAGAAAAAAGTCGGGCGGAACGGGATCGGGCCTGCCAAACTCGAAACAATCGACGAAGAAGGCAACTCTAC |
| | CAAGAGCACCGCTTCGATGTCGGTCGAAGCGGCGGTAATGATCTATCACCAAATGAAAGCGGATCGATGCCG |
| | AGCGGCAACCGCTAAAAAAACGCTGATCGACGCAGGGGCTCTTTCAGCCCCCCTAACCGCCAAAGATATCAA |
| | ACGTGGCGATCGCACTCTCACCATCACCGAACTGATGGATATGGCGGGCAGGATTACCGACCCGACTATCAGG |
| | GCGATCTACCACCAAGTCGAGATGTTGGTCAACGAGTTGATCGCCCGCTTCGGTAAACCCGAACGCATCGTTA |
| | TCGAAGCCCAAAAGGAAATCGGGCGAAGCATCGAAGACATCGAGAAAGCGATGGCAAGAGAGCGAGAAAAG |
| | CATATCGAACGGCAACGAGAGAATCGAGCCCGCAACGCCGCAATGGGTACCAAAGCCCGCTTTGCCCGCCTG |
| | TGCGCCATCAGGGGTGATCGATGCTTTATCAGCGACCGACCCGCAGCCGAAGTCGGCCACCTGATCGCCGATT |
| | CGATCGGGGGCACGCTTGAAATGGCTAACCTGATCCCGATCGACCCTGCCATCAATAAAGAGATGGGCAACC |
| | GCACTCCCTACGAAGCCTTTCGAAAGACTGAGTATTGGTCGATCATCCAGCGCAAACTGCAAGCGCTTGAAGA |
| | CGAAGTGAAGGCTTTGAAACCGCCGAAAGGGACAAAAGGAACGGCGTGGACGATCTATCACCGAGCCAAGC |
| | ATCAGTTTGATTTTTTCGCTTGGCGATTTCAATCGAATGCGAGAGAAACCCATCAAAGGAACTTTCGCCCCGGC |
| | TCGCTTGACGACCTGCGGTGGATCGAAAACTTGCTCTTTCTCGGCGTTGCCCCGATTTGCGACAATATCCGAAT |
| | CGTCAGTGGGCGAACAACCGAGCGCATTCGCCGAGAGATACTTGGAATGGACAAAGACCGCCGAGACCATCG |
| | CCACCATGCGCTCGATGCGCTTGCTATCATGCTCGCCAATCCTTTGAAGCCGTGGGATTTGAAATCGAGCAATT |
| | CGCTCGGTATCCCGCTCGGCCGAATCAAACAAGCCTTCGCCGACGCTGTCGTCTCGCAAAAGCAAGACCACTC |
| | GCTTCGCACTGCGTTGCATAAAGAGAACGCGATCCCGAAGACCAAGCGGGGGGCTGCATATCGAAAAATCGG |
| | AACGGGGGCGAGCGAACGCGTAGTCGACACCCAATCAAAGGCCTATTGCGAAGTTTGGGCGTTGCCTAACGG |
| | GAAGTGGGAAGCGGTCGTAGTGTCGAGTTTCGACGCTGCGCAAAAGAACTACCGGCAAGGGATTGACCATCG |
| | CCCCCATCCCGCCGCTCGACTGGTTATGCGCTTGTTCAAATCCGATTTGCTCGGTATCGGGGGCAAGATATACC |
| | GGGTTCAGGAACTACTCGGTTCTGGCTCGATCTACTTGGTCGATCATCGATTCGCTGGCACTATTCGAGACGCC |
| | CGCGCAGTTTGTAAGACGGGCGTCAATGTCGATTTCTTTAGCAAAGGGGGTGATTCATTGCGCAAGGCGGGGG |
| | CTCGTCTTGTTTCGATTCGTAAGAGTTGGGTGGGATCG (SEQ ID NO: 35 |
| | 1 ctaccccgca aggaaggagc cgccgaccga tgtcttgtta ggaaaactaa tccttcggaa |
| | 61 attagtcgag aaggggctcg cccccccaaa agccaacctg tcgtggcaat gcatcgaaag |
| | 121 cctgaccatc accccgtgga tatgggatcg cttcgccgag cacgggatcg aagtcgcgct |
| | 181 cggcgagatg gtcaggcggc ggggcgaaga gaaatcaaaa aaaaatcgaa cggggggttg |
| | 241 acttttggat aatagccttc ataatctcgc ccatcgatag ccgagcaacg aagttggatc |
| | 301 aggacgtcat tcttcgaatt cgaaaacgcg gggattgctc gaatgggtgg atgatatgtt |
| | 361 ggatcaggac gtcacttttc gaattcgaaa accgatctcg cctaagtgat tgattcagcg |
| | 421 aagaaattgt agaatcgcag ttgcaaaaaa tgatgtcctg atccaataag gggcgcagcc |
| | 481 cccgcaccat ttttccccga tctacggatc ggatcggcgc tgccttcgaa aagccgacat |
| | 541 ggaaaacccc cagtttatcg gggacgatgt aaacctgagt cgccacggcg ctcatctcgc |
| | 601 ccgctctacg gaccgggctt cgctggaaaa tgggagccgt gaccatgaag cagctgaccc |
| | 661 tggatgtggg aatcgcctct atcggctggg ccatcgtgtc caagaaaggc aatgtgaagg |
| | 721 ccggcagccg gatcttccct gatgccaagt ctggcagaga gagcaaccag tctagaaggg |
| | 781 ccgccagact gatgcggaga ggctacagac ggaaggccaa gcggagagcc gatacactgg |
| | 841 ccatcatccg ctctattcac cccggcttcg atcccgaagg acaccccgat attgagagag |
| | 901 aggccctgat caaggccatc attacccctg gcgctcccac accttctctg gatcaactgg |
| | 961 cctgcgcctt ccagagattc gccaagtcta gatggcccca gtacagcaga accctgccta |
| | 1021 agagaaccga aagagaggac cagttcatcc aagtgtgggt tatcgccgag cggacatacc |
| | 1081 ccgacagatt cacccctgat gtggccacca gactgctgca ggccatcttc tttcagcggc |
| | 1141 ccatcaagga cggcgacaga gccaagtgcc agctgtttag acaccacggc gataaggccc |
| | 1201 ctctcgtcgg atggacacac gagcctgagc tgcagagatt tgccatcctg agcgacctgt |
| | 1261 ccaacctgac catcggcatc ggcagcaccg acaatctgct gtgcgagtac cctgacatca |
| | 1321 tcgaggacct ggaaacccgg tgcttcgaga caggcatgag ctggcgggaa attgccgagc |
| | 1381 acgtgaaaga agtgatcggc aaaggcgtgg tgttccgggg catcgacggc cagaaaaaag |
| | 1441 tgggcagaaa cggcatcgga cccgccaagc tggaaaccat cgacgaggaa ggcaacagca |
| | 1501 ccaagagcac cgccagcatg tctgtggaag ccgccgtgat gatctaccac cagatgaagg |
| | 1561 ccgacagatg cagagccgcc accgccaaga aaaccctgat tgatgctggc gccctgagcg |
| | 1621 cccctctgac cgccaaagat attaagcggg gcgatagaac cctgaccatt accgagctga |
| | 1681 tggacatggc cggcagaatc acagacccca ccatcagagc catctatcac caggtggaaa |
| | 1741 tgctggtcaa cgagctgatc gccagattcg gcaagcccga gagaatcgtg atcgaggccc |
| | 1801 agaaagagat cggccggtct atcgaggaca tcgagaaagc catggccaga gagcgcgaga |
| | 1861 agcacatcga gagacagaga gagaacagag cccggaacgc cgccatggga acaaaggcca |
| | 1921 gatttgctag actgtgcgcc atcagaggcg atcggtgctt catcagcgat aggcctgctg |
| | 1981 ccgaagtggg acacctgatc gccgatagca tcggaggcac actggaaatg gccaatctga |
| | 2041 tccccatcga tcccgccatc aacaaagaga tgggcaacag aaccccttac gaggccttca |
| | 2101 gaaagaccga gtattggagc atcatccagc ggaaactgca ggccctggaa gatgaagtga |
| | 2161 aggccctgaa gccacctaag ggcaccaagg gaacagcctg gaccatctac cacagggcca |
| | 2221 agcaccagtt cgatttcttc gcctggcggt tccagagcaa cgccagagag acacaccagc |
| | 2281 ggaacttcag acctggcagc ctggatgacc tgcggtggat cgagaacctg ctgttcctgg |
| | 2341 gagtcgcccc tatctgcgac aacatcagaa tcgtgtccgg cagaaccacc gagcggatca |
| | 2401 gacgggaaat cctcggcatg gacaaggaca gacgggacca cagacaccat gctctggatg |
| | 2461 ccctggccat tatgctggcc aatcctctga agccctggga cctgaagtcc agcaactctc |
| | 2521 tgggcatccc tctgggaaga atcaagcagg cttttgccga cgctgtggtg tcccagaagc |
| | 2581 aggaccactc tctgagaaca gccctgcaca aagagaacgc tatccccaag accaagaggg |
| | 2641 gagccgccta cagaaagatc ggaacaggcg ccagcgaaag agtggtggac acacagtcta |
| | 2701 aggcctactg tgaagtgtgg gccctgccaa acggaaagtg ggaagctgtg gtggtgtcca |
| | 2761 gcttcgacgc cgctcagaag aactaccggc agggcatcga tcacaggcct catcctgctg |
| | 2821 ctcggctggt catgagactg ttcaagagcg atctgctcgg aatcggcggc aagatctacc |
| | 2881 gggtgcaaga gctgctcgga tctggcagca tctacctggt ggatcacaga ttcgccggca |
| | 2941 ccatcaggga tgctagagcc gtgtgtaaaa ccggcgtgaa cgtggacttc ttcagcaaag |
| | 3001 gcggcgatag cctgagaaaa gccggcgcaa gactggtgtc tatcagaaag agctgggtcg |
| | 3061 gaagctgagt cacttttcga attcggaaac ttttccgacc gacaagtact cgatcatcta |
| | 3121 gttggatcag gacgtcatgc ttcgaattcg aaaaccattc gatgccgatg cctacaccgc |
| | 3181 aaagagttgg atcaggacgt catgcttcga attcgaaaac cttcgtttca ataacc |
| | (SEQ ID NO: 36) |
| Cas9t | GTTGGATCAGGACGTCATTCTAAAATTGCAAAAAATGATGTCCTGATCCAATAAGGGGCGCAGCCCCCGCACC |
| single | ATTTTTCCCCGATCTACGGATCGGATCGGCGCTGCCTTCGAAAAGCCGACATGGAAAACCCCCAGTTTATCGG |
| guide | GGACGATGTAAACCTGAGTCGCCACGGCGCTCATCTCGCCCGCTCTACGGACCGGGCTTCG |
| scaffold | (SEQ ID NO: 37) |
| Cas9t_ | GTTGGATCAGGACGTCATTCT (SEQ ID NO: 38) |
| processed | |
| DR | |
| Cas9t_tr | TTGCAAAAAATGATGTCCTGATCCAATAAGGGGCGCAGCCCCCGCACCATTTTTCCCCGATCTACGGATCGGA |
| acr | TCGGCGCTGCCTTCGAAAAGCCGACATGGAAAACCCCCAGTTTATCGGGGACGATGTAAACCTGAGTCGCCAC |
| | GGCGCTCATCTCGCCCGCTCTACGGACCGGGCTTCG (SEQ ID NO: 39) |

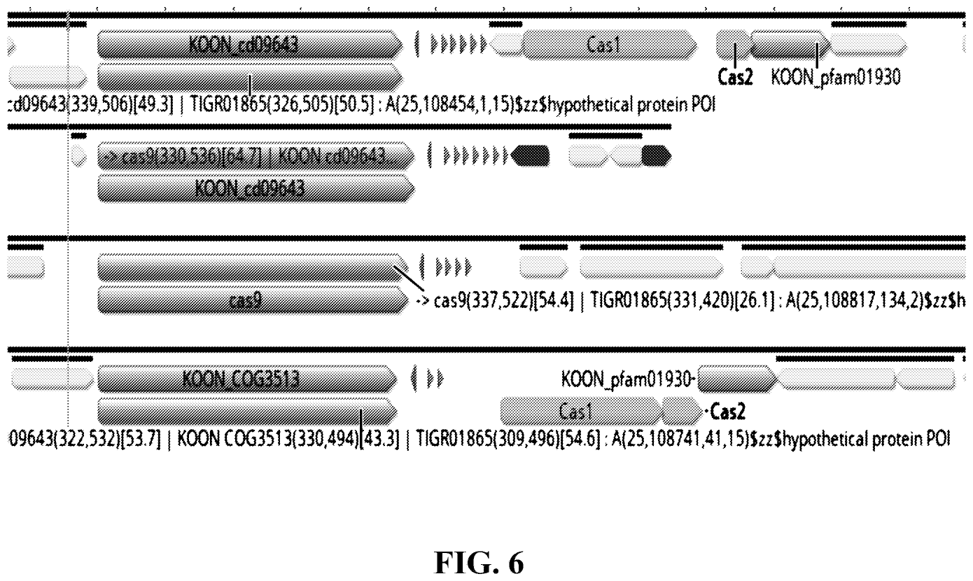

Some exemplary small Type II-B Cas9 proteins are shown in FIG. 6. Relevant sequences of the exemplary small Type II-B Cas9 are shown in Table 11 below.

TABLE 11

| | |
|---|---|
| MGQA01000 | 1 attgggtggg atagcatgga tttgtaatcc ttttccctca gctcagcgaa ttttttatat |
| 002.1_ | 61 cttggatcct ttgatacatc ttttacatag attggctttc cttcttttgc cgctgatcct |
| organized | 121 gtaactcctt ctcccaattt gagccttacc ttgccaatcg cctcttttac aaagcttgtg |
| Deltaproteo | 181 gttgccgtta agacaagttc ttttccattc cagaggtata ttgaggaaac atctacgccc |
| bacteria | 241 aatctctttg cggtcttttc tacgactgac tggagcacct catcaagcgc ataatcagag |
| bacterium | 301 cttgcaagca tgcttatctc ttcaagggtc ataatctcca ttttttccct gttcgccctt |
| GWF2_42_12 | 361 tccatctgaa agacaatgcc gccttcttca ctaatcttta catgagaaaa agcatcgctt |
| gwf2_ | 421 ggataatatg gattgatagc cttgcttgct gacggcattg tcttatattc ccaaaaaccg |
| scaffold | 481 catgcgcaca caagctttac cctgtaatcg tcaaccctct cgcttttcat cttctttccg |
| 1015, whole | 541 catttcctgc atcgtgtaat aaattccatt cagcgcctcg ttaaaatcag ttggaattta |
| genome | 601 acaggctgtt gaaaaagcca tcaacagcct tgattacaca gattagagaa aaagattaca |
| shotgun | 661 cagatattta cagaaatttc aaggagtttt aatctgtgta atcttgtctt ttatctgtgt |
| sequence | 721 aatcccgaat agtcgggatg taatcggaga ttgttgagtt tttcaacaag ctcttaatat |
| | 781 tcgggagtcg gttcttcagt ctataacctg aggtatttct ttgttctctt ccgaaatatt |
| | 841 aggcaagacg agttttacga ccttgccttc gtcaccaagt ctgccaagtg gattgccatt |
| | 901 cagcgtcaca ttgacaccgc ctgcattccc tatcagtata gaaaattttt cttttgctat |
| | 961 ccatttaacc ttttcgcctt ctctgagaga tacctcaaac ggatcctgac tatccacttc |
| | 1021 agctcgaatc catgtgcttt tcgtggcttg tataatcaag gccatgggtt cttttaagtc |
| | 1081 tgccttagat gacacagatt cttttgtatt tttatcccct ttttgtaaag ccgaagggct |
| | 1141 aacaagtatg cctctatcgt cttcctgttt agaccgggcc tcgtaagcta tagcttcctt |
| | 1201 cttgttgcta tcagatacat cgttgccagt cgttagctgt atttgtgaat aatcaggagc |
| | 1261 atctggttta tactttttta agattacata tgctccagct gaaattatta aaaggatagt |
| | 1321 gacagctata gcaataagcg attgcgtaga taatgtggtt agtctctcta acttttcgtc |
| | 1381 ttttgctttg gcagcctcaa caggttcgac cttactttca gtaagggttt tcatatacac |
| | 1441 ttcataacga agcagcgcat catgtccgtc tacaccaaga tgtttgcaat aagcctgtat |
| | 1501 aaaacctttt acaaaaggcg gtgcgggtaa tttgctaaaa tcgtcttttt ctaatgcatt |
| | 1561 aagcagccca gcccttattt tagttatttt agccacatct tgtaatgaga tacctctaat |
| | 1621 ttctctttct cttttaagat attcgcctat gctctccatt ttgctaactc ctaattattt |
| | 1681 atgttgaagg cttttgccta tttaatgttt attatttcaa gaggtcaata tatcccttag |
| | 1741 ccgaccttgc catctcgctg tcggggacaa gtttaataac ttcttggaat gcattcgccg |
| | 1801 cgcctttctt gtctttaatc tttatgagaa caagaccgag gttgtaatgc gcatccgcat |
| | 1861 aatttggcgc gttcttaatt acgagtctga aagtatcagc tgcctcttta tctttattca |
| | 1921 ttctcatata ggtaaggcca aggctattat atgcctgcaa atacattgaa ttgtttttaa |
| | 1981 ttgccttttt aaaactctct tctgcctcta cataatctgt tttttcgtaa aatgctcgtc |
| | 2041 ccatattaaa ataggcaagc tctggagttg tataaaggat gtttgcgagc gctttttggg |
| | 2101 ccgctgcgat agcagcatcc cactcttttt tttcaatgta gacagctgag aggtttgtgc |
| | 2161 tggcatcaga aaattttggg tctattcgga tggccttttt aaagtgcttt atcgcatcat |
| | 2221 catggaggcc tttcgcaaaa taagcaagcc ccagggcatt atgatacgat gcctcatcag |
| | 2281 gatatttttc aacagcagtt atcagttctt tcagggcttc agggatattt ctttcattga |
| | 2341 gatgaacaac cccaagctta taatgtatat ctgcatcttc ctttctattt gcctttgagc |
| | 2401 ctgcacatcc gtaaaagaca gttgccagga ggcagtaagc ggtaagcagt aagcagtggg |
| | 2461 taataaacag taactgccta ctgtatactg cgtactgcat actttctttg actgctggtt |
| | 2521 tctgacttcc agtttctaaa ttcttaatca tctcatactc cttagcctct tgtcggtttt |
| | 2581 caagatatat cttcaaaatg ggttaattgc ttaaattttc tatatctatc gtaaatctct |
| | 2641 tttagtgtca gggtgcgcat tctgttgagg ctgaaatcct ctacattaaa tgaggccatg |
| | 2701 acactcccaa agataattgc ctgtcttata ttagattcat ttgtgttgtc aatatttgca |
| | 2761 agatatccta tcagtcctcc agcaaaactg tcgccagcgc cggttgggtc aaatacagat |
| | 2821 tccaggggat aggcaggtgc tgaaaatata gaaccaccat tgaacatcaa tgcgccgtat |
| | 2881 tcccctcttt ttattattac tgttttaggg ccatatgcaa gaattttttt tgctgccttt |
| | 2941 acaaggttgg gttcttttgc aaactctcgc gcctcacctt cattcaggac aaacaggtcg |
| | 3001 actttcgaca agagcccctt aagcgcatca ggcttgcctt ctatccagaa attcattgta |
| | 3061 tcacatgcaa cgaattttgg ctttttgact tgttcgagaa cattcaattg aatctctggg |
| | 3121 tcaatatttg caagaaacac atagggtgat tttgtataga ccgctggaat atgaggttta |
| | 3181 aattttttaa aaacatttag gtgagttgca atggtatggg cttcgtttaa atcatactca |
| | 3241 tatcgcccct gccagcgaaa tgtcttgcca gctatttttt gtaagccgtt tgtatctacc |
| | 3301 cctttttctt ttaaaaaatt aatatgctca tctggaaaat cttcacctat aacagctacg |
| | 3361 aggctgacat ctgtaaaata actggcagcc gtagaaaaat aagtggctga accgccaagg |
| | 3421 acattgtctg cctttccaaa aggtgtttca actgaatcaa gtgcaacaga acctacaacc |
| | 3481 aggatgccca tcaaaatcct cctgaatttc aagtaggcag tatgcagtaa ggagtaagca |
| | 3541 gaaagaaacc tgcctactgc ctaccgctta ctgcttactg ttttttacca ttattcatcc |
| | 3601 acactgtcaa ttaatgtgga aagggcaact atctcaactt ccctcttgag cattggtacc |
| | 3661 atctctttta tagctaaaat ggtggtatgg tgtggatggc ctatagcaat ggcagagcct |
| | 3721 ctcttttttg caacttctat caattctctt atctgtccct ttatgtaatc tatatcctct |
| | 3781 ttgttatcta aaaatacctg cctgctcaat gttttaagtc cgagctcttt tgctaatcta |
| | 3841 taggcagttg atttattcgt ggttttgcta tccagaaaga atagattttt ttcttttgcg |
| | 3901 atttccaata ctatcttcat aagtctttca ttctctgtaa atttcgagcc catatgattg |
| | 3961 tttattccaa taatatgcgg gattgcgtca atatcctttt taacctgtga agcaacctgt |
| | 4021 ttttcagaca tggttgtaag tattgcccct tcccctggat cgttgttgct taaatctttt |
| | 4081 ggctccatgg ggagatgcag caacacctct cgcccgttta aataagcctc ctttgcagcg |
| | 4141 ttctctgaat tcggcaaaaa tggcaataca gcgatagcaa tgggggcatc tatttctaat |
| | 4201 atctctttaa gctgtgccct gtcgcggcca agatcgtcta taattattgc taccttagcc |
| | 4261 agtggtatat gcggcttttg gggaacagga ggttgaggtt tctctatctt tgctacaact |
| | 4321 gtagatgttg aaataggttc tggaggtttt tcttttgtac tgtagctgag gtatagaata |
| | 4381 aaccctaata tagcaatgat aaaaaatacc gttgcaattc ccaaaacaaa aagggtcttt |
| | 4441 gatgagaaag accctttttt gctccagtta tttttagcca ttttttaacc tatatagcta |
| | 4501 atccctctta gatgagccac tcttatgatt ccttctttat tgtctcttta aatatatacc |
| | 4561 aactcttcag ataatccaat gcccttttga gctgcatgtc cgcttctgtc tcttcctttt |
| | 4621 cttttactac cttttcctct atctttatct ttttttcttt aggagattca acctcttcgg |
| | 4681 cttcaaggtg tctttctagt tccttttctt taatatgttc ttttattgtt tctccaacca |
| | 4741 caatatcggg ttctatgcct tttgcctgta tagatcttcc taaaggcgta taatatttag |
| | 4801 aggttgtaag tctaaccgct gaaccatcgc ctaaaggaat gatagtttga acagaaccct |
| | 4861 tgccaaatgt ctgggttcca agcacaaccg cccttttatg gtcctggagc gcgcctgcaa |
| | 4921 caatttctga tgcgcttgcg cttccaccat ttaccaggac tataatcgta tattgggggt |
| | 4981 gcgttccatc tgcccgtgcc tcaaacttca tatcctggcc tgggcttctg cccttggtat |
| | 5041 atacgattaa acctgattcc aaaaacgtgt cagctactcc tacagcctgc tgcaaaagtc |
| | 5101 caccaggatt attccttaag tcaaggatga gaccttttaa cttgctgtcc cttgagccaa |
| | 5161 gcttatttag cgcctcttca agttcattag tagtcttttc ctggaaatgt gcgattcgga |
| | 5221 cataaccaaa cccttcttct aacgttttat attttacact cttaatagct atgatatccc |
| | 5281 ttacaagtgt aaattcctgt ggttctttaa agtcctcccg cattatccag atagtaacct |
| | 5341 ttgttccttt ttggcctcgc attagcttta cagcatcgtt tatggtcata tcctttgtcg |
| | 5401 gcttatcacc tatttttact attttatctc cagcctttat cccagccctg aatgctggcg |
| | 5461 tatcttctat gggcgcaatg acagtaatta tgccatccct tattcccatc tctatgccaa |
| | 5521 taccaccaaa ggcgcctttg gtttcaacct gcatttcttt atactcatct ggagccataa |
| | 5581 aagacgaatg cgggtcgagc ccgagaagca tgccttttat tgcgccataa atgaggtttt |
| | 5641 tggggtcaac atcttctgca tagttatcct ctacgataga gatcacgtcg gtaaatattt |
| | 5701 ttagattctc atatatcttt ggaggcacag cggaaaccct gtgattaatc ccccatagaa |
| | 5761 gaacgctcgt tattagagct attcccaaca gcgtcatgag cttacgccct tttaacctct |
| | 5821 tgaacataaa tgtattcctc ctgagtttat ttaagataaa gacttgatgc gatatgcgaa |
| | 5881 agggttaaag gaggtagcgc tacctaattg ccttctgcct taagcctgac gctctctgtc |
| | 5941 ttatcatatg tcaaccaatt taccgggtct aatggtacac ccttctgtcg tatctcaaaa |
| | 6001 tacagatgag gtccttttat tgaacctgta tctcctacaa gtgcgattat atcgtctctt |
| | 6061 tccacggtag aattaacctc tgcgaggatt tttgacaaac ggccaaagag tgtatgatac |
| | 6121 ccattgccgt gatctataat caaaaccttt ccatagcccc tgaaccatcc agaatataca |
| | 6181 acgtggcctt tatatacgga cttaacctca gcccccatcg cagcctcaac ctcgatgccg |
| | 6241 ttattaaagg taacagtgtg aaatttggga tgctctacct ttccatacat tgaaatgact |
| | 6301 ttgccagcta caggcattgg caaatgaccg cgcatagacg caaaaccgtt cgtctcatta |
| | 6361 tccaaaggat tattaaccgt ctcattcttc aacctatcta tgaatagttg aagctctctg |
| | 6421 cctgcgcttt ccatttcctc aattgcggca atctgtattt cctttttctt ttttatatca |
| | 6481 tttaaaagga cctttttctt gtctttttct tcttttattt cttccttttt cttttcaact |
| | 6541 gcatctttta aagagcctat ctcctcctga aactctttga gccgtatttt ctctgcttct |
| | 6601 aagagcagtt gattctcatt gtaatctttt aacagggttg tatcatgatc tataaccttg |
| | 6661 tttacatatt tatatcttct actaaactca ttggcaaaat ttggggcaaa aatgctctgg |
| | 6721 gttatgcccg ccctattcat cttgtacatt gcaacaagcc tttgttttag aagttccatc |
| | 6781 aaccttattc tctcctgaat caactctttt attcggatat ccacatcaga gtttttttgc |
| | 6841 tttatgacaa caaggctgtt ttctatcttt tctaattcct cctccttttt agataaggcc |
| | 6901 ttgtcaagcc tttcaagtcc ttcaagaata gcatattctt tttttacatt atactctaaa |
| | 6961 cttctctttt tttctacaat ctcccttttg agggattcca gttgtttttc cttttttaca |
| | 7021 atctccttct ttagcttggc aggttcagcc gcataaccat gaatggccgg ggaaacgagt |
| | 7081 aaacacaaaa caagatagaa ggggtaggtt ctaagggtca ataacaataa taggtttaaa |
| | 7141 ttatggattg ccacagtttt aactttctct ttttaagcag tcattggttt tttatagaat |
| | 7201 ttaatttatc ctatctaaaa caattaaatt tatactcttt attctatctt tcagcgcccc |
| | 7261 catttttgaa tctttgccaa gctcgaggtt tgtaacaaca gctttattta cagttctgaa |
| | 7321 accatcttta tcataatagc caaaaagctg atggagcgca tagtagtttc catctacgtt |
| | 7381 gccgagatag agcatgatat gaccgttgag ggctaataag gtaatgcctg ggattgcgga |
| | 7441 acttattgcg ttatcaatat ctccgtttgc cggcatttca tcaaaacctg ctaacagcgt |
| | 7501 cccgacatta gattgtttgc cagaatgcct gggaaggttt atgcccatcg tcgcaaacac |
| | 7561 atccttgata aagcttgaac agtccctcaa accgtttcta cctccccagc tgtatttctc |
| | 7621 gccaaggatt ttaaatgcct gttttatcac atttttcttg gtatagggaa gatagccaat |
| | 7681 attcacatcg ctgcctttct taataaatgc atctgcccat tgaagctgac cattgcccat |
| | 7741 tttttttgga aatttaataa tccaatgtct tttatcttca ccgctaagcg caaaggaacg |
| | 7801 acccatggga atggactcaa gaacctttgc acccttattg tctgcaaaaa ctgccactcg |
| | 7861 gtttcctgtt attaaaacga atttttcagc atctattatt aaatcgtctt tgctatttac |
| | 7921 aaaagcaatg atattgagtt ttatccagcc acgtacaatg tttgtctgaa agaaacccca |
| | 7981 ctgtccatcc ctactcttgt gcaggagcgc caccacctga ggaggatata tggcggaata |
| | 8041 ctgaaatgca tcaaaatctt tggcagaagg gcctttaaac acaggttcat cagtagggaa |
| | 8101 tgaccttata tccgtccttt tagttacaat cccaaatgta agttcattcg attcatttat |
| | 8161 agcctcaaaa ttcatattgt ctaatagttc ttcataaaag gcgtctttta caggttttcc |
| | 8221 cttcttatca aacatatttt tatctactgg gagagggtct tcaagcagcc atttttcaag |
| | 8281 ctcttctttt gataccgatg ttcccatttt tattacatct gccatttcat tgttttcgcc |
| | 8341 aagaatcaaa gcgtttaatt ctgcaatttt ctctgaagaa agtattacct catcaggatt |
| | 8401 atcaagcctt ccaatccaga agtctgcttt aaatattgga aggtaggcgc ttgcttcgag |
| | 8461 caatacggga gagagaaaaa ttatactgat gactatcttc aaaactttca tgccaacttt |
| | 8521 gataacataa tacgtcaata ttctcaagtc ataaattatg ttcggtaaaa tcaatgtcta |
| | 8581 tttttgggta tgtttcaagc tctggacaat aaaatactac tgtgatatac ggagtctgtg |
| | 8641 gaaaagatta gaaatcctat tccaacagtt gatataataa ttgaattaaa aatcccccct |
| | 8701 cacccccctt tttcaaaggg gggaaggggg gattggggga ttgtccttat agagaggaaa |
| | 8761 aatcccccgt ttggctgggc aattccaggc ggttttgtgg attatggaga aaccttggaa |
| | 8821 gatgcggcta tacgagaagc tcatgaagag acagccttga aggtgagact aaaatgccag |
| | 8881 ttacattctt attcaaaccc tacgagggat ccccgatttc atacaatttc cacagtattt |
| | 8941 gttgcagagg cagatggaac accgcaagcc agagatgacg caaaaggaat cggcgttttc |
| | 9001 acagaagaca atctgcctaa gcctctggcc tttgaccata agaagatatt ggaagattat |
| | 9061 ttcagatgga agaaggaagg gtggagggtg tttgaggaaa aggcattgca gtcaaatgat |
| | 9121 ggctgtgata acttgcttag ctgaaaggta attggattat gataaaagat acccaactat |
| | 9181 taaaaaaatt tgaagacaca atcatgaaaa aagagggaag gctttctttt agttactcta |
| | 9241 tgcgcatatt tgaatctctc tggaatgaag ggataaaact cggaatattg cctcccaaga |
| | 9301 aaccattaga aggaatagaa gttgatataa agatagcaca ggtgctcaat tcatgtttga |
| | 9361 agaaatcctc gcaaggatag gcgcagcgct tgttaagtgc aatattccct acatgattat |
| | 9421 aggcggacag gctgtattgc tgtatggaga accgcggctc acaagagata ttgatataac |
| | 9481 cctcggcgtt gatacaaatc accttaatca acttcttgca atccttcaaa aaataccact |
| | 9541 caagcccata ccagaagaca tagaatcatt cgtcaagcag actatggtct tgccggcgct |
| | 9601 ggatgaaact acaggcatac gggttgactt tatattctca tttacgcctt atgaaacaga |
| | 9661 ggcgataaag agggcaaaga gtataaatat tctcaatcag gaagtatgct ttgcctctgt |
| | 9721 agaagatgtc ataatacaca aaatatttgc aggacggccg agggatttag aggatgcaag |
| | 9781 gacaatcctc ttaaaaaatc ctgatattga cattagatat atacagggtt ggttaaagga |
| | 9841 gtttgacgca tcaattggca ataaggattt tctgaaaacc tttgaggaca tattaaaaac |
| | 9901 cataaatcag tagaataggc ttgtttttag tggtaaaagg aaggacagaa ggtttttgag |
| | 9961 atatggtgat ccccatcgtc ataatcaaag gtaaataaaa atggaaagag aacttgtttt |
| | 10021 aggcattgat tacggcggca aatataccgg tcttgcagtt gttgaccgaa ggcataatca |
| | 10081 ggtattatat gcaaacagac tgaagatgcg ggacgatgtc gctggtattc tcaaagatcg |
| | 10141 cagaaagcaa agaggtatca gaagaaccgc tcaaaccaag aaaaagcggc tgcgtgaatt |
| | 10201 aaaaaactat ctaaaaagta ttggatataa cgaatccaca gcaacatttg aaacggttta |
| | 10261 cagccttgcc cataaaagag gatatgatta tgctgatatg cctgaagaaa agacttccga |
| | 10321 agaaatagag gcaatggatg tggaagagcg caaacagtgg gaaaaagaga aacaggaatg |
| | 10381 ggaagagaca aaaagaaatt caaggcacag gaaagaagtc gtaaaagatgtccataaagc |
| | 10441 aatgattgaa ggaagagcaa ccgaggagca gataaaaagg gttgaaagga tttttaataa |
| | 10501 acagtaccga cctaaaagat tcaataatag gattttaact aaatgtaagg tagaagattg |
| | 10561 cggagttaat acgccgctca gaaagaatgt gagagatttg cttatagaaa atatagtcag |
| | 10621 atttttccct atagaacaat ctgaaaagga taatctcaaa gatgctgtgc ttgataaaaa |
| | 10681 caggagggaa gaagttaaat catttttcag aaaacataag actgatgaac atattaggaa |
| | 10741 gcaggtttat gatatagcgg ataacaagct ttcgggtaga acggtctttt gcaaagaaca |
| | 10801 tatccttgaa catactgagc atagcaaaga ggaacgaaag gttttccgtc ttgcgccatc |
| | 10861 cttaaaaaca aaaatagaga atgtccttgc tgttattaaa gacgagatat tgcccaaatt |
| | 10921 tactgtcaat aaggtggtaa tggagagcaa caattttgat atagcagcca aaacacaggg |
| | 10981 gaaaaaacga ttggcaaaag aagaatatgg taaagggccc agggaaggca aagagaccag |
| | 11041 aaaggaagcc cttcttcgag aaacggatgg tagatgtatc tactgtggaa aatctattga |
| | 11101 catatccaat gcgcatgatg accatatatt tccccgaaag gccgggggct taaatatctt |
| | 11161 tgcaaattta gtagcatgtt gcgctgtatg taatgagaat aaaaaaggga gaactccgtt |
| | 11221 agaaagtgga atctcgccta aacccgaaat aatagccttc atgaagaatg atttaaagaa |
| | 11281 aaagattctt gaggacgcac gaaatatcaa tacagtagat tttaataaat acatgtccca |
| | 11341 tgcctctatc ggctggagat atatgaggga cagactaaga gagtctgccg gcaataaaaa |
| | 11401 gttgcccata gagaggcaga gcggcatata cactgcatat ttcaggaggt ggtggggatt |
| | 11461 taaaaaggaa agaggcaaca cccttcatca tgcccttgat gctgtaatcc ttgcttcaag |
| | 11521 gaaagggtat agtgatgatg gattggtgga tatgaccctt aagccaaaat ataataaagg |
| | 11581 tggagggttt gactctgaaa aacaccttcc tgaaccgata gaatttaaaa gggataaagg |
| | 11641 cagcaagggc tctgcattgc atgatagaaa cccactgtct tataagaagg gcattattac |
| | 11701 cagaagattt atggttacag agattgagtg cggtaaagaa gacgatgtta tttccgagac |
| | 11761 atatcgtgaa aaactaaaag aggcttttaa acgatttgat acgaaaaaag gaaaatgttt |
| | 11821 aacagacaag gaggcaaagg aagcaggttt ttgcattaag aagaatgaat tggttatgag |
| | 11881 ccttaaatgc agcattaaag gaaccgggcc agggcagatg ataagaatca ataataatgt |
| | 11941 ttttaagacg aatgtccata atgttggcgt ggatgtttat cttgatgaaa aaggtaagaa |
| | 12001 aaaagcatat gagcgaaaga atccgagact gtcaaaacac ttcatagaac caccaccgca |
| | 12061 gcctaatggg agagtttcat ttaccttaaa acgaagagat atggttacag ttgaaggtga |
| | 12121 agacgcaatc tatagaataa aaaaactcgg aacatctcct acgatagaag cagttgttgg |
| | 12181 tagtgatgga aaaacaagaa ctgtctctgc caccaagctg acaaaagcaa acagtgctga |
| | 12241 atagttttta aaattaaatg tcctaaccca cgatgaaggc gccatttctg gcatagaggc |
| | 12301 tcaggctgga caaacccaag cgtatatccc ttcactgtat aggcgtttta caattaaggc |
| | 12361 tcttttaaaa tttcgcaaac cccttaaggt ttgtttcaaa aaatggtctt gcggaaattt |
| | 12421 tataaattgt ttttgaaaat ccctttgaaa ttccagcaaa acaatagctg tttcacgcca |
| | 12481 tattttcaaa gagccttaac tgtaacttcg atcttctcga aggagatatg cgtgggcaga |
| | 12541 tgtttcacgc catattttca aagagcctta actgtaactg tgtgaagcgg tgcaaggcgc |
| | 12601 atcccgcctt tataagtttc acgccatatt ttcaaagagc cttaactgta actccttctt |
| | 12661 atacctgcgg taggccgcct gctcagaagt ttcacgccat attttcaaag agccttaact |
| | 12721 gtaacgttcc tcaatctgtg cggaaggccg ccccgaaagt tgtttcacgc catattttca |
| | 12781 aagagcctta actgtaacga ttgaaaatgg caaccatgag ctcggatttg tgtatgtttc |
| | 12841 acgccatatt ttcaaagagc cttaactgta acttcataat accacactga aacgaaactc |
| | 12901 atgctaatga tacttcggct gtcttgcctg tagattcatt aataacaagg agtttaccct |
| | 12961 catcctcttt tttcaactgt tcattggcat tcagcaattc tatgccatag accgttccat |
| | 13021 caggcgccaa atctatgttc agttcatcgc tgattttcac agtctccact tgtgctggtt |
| | 13081 tttcatgcag gcgaatataa gctatgttgt atctcggatc atatgttaac ttcatatatg |
| | 13141 ttccaccgta aggcttgcaa tatggcgtat actgctttat agttacacca cataacctca |
| | 13201 aaaatcaaga aaatattgag gaggtctata tggataatga aacatcaaaa atcctccgca |
| | 13261 tccatccgga atatcaaccg gaggaaaaac tcccgccagc acagttcaat ctctttacag |
| | 13321 gagaagcaga gccagtcccg caactgattg acaaaaatat tgatgatgaa atatttgttg |
| | 13381 acgatgggat ttcgcttgtg cagagcggag attcgtcgca aatagttctg tcaggttttg |
| | 13441 gcatcttctt aagtaaaaag agtgaacgcc ttattgtaaa aaagagcagt aaggttattt |
| | 13501 atgaatttcc cttttttaga ctgaatgagg ttattgtcgc ctctcgtggg atgtcattat |
| | 13561 cctctgacct tgttgaagag ttatgccaaa gggggataag actgaatttt ttgacaaatg |
| | 13621 gcggcaaacc gtatgcaatg ctttcctcgc ccatgctctc cgcaacggtt gtatccagaa |
| | 13681 gagagcagat tatggcgttt aatgatgaaa gggggcttga attctcaaag gcggttgtag |
| | 13741 aaggaaagat tgtaaatcag gaaaggctta taagatattt tggcaagtat attaagaccg |
| | 13801 tagatattga cagatttaat aaacttgaag gaattgctga tgatattaaa aagatgagcg |
| | 13861 gacaggtaga taatattgat gggcaaaata ttgaagatgc aagaggtggt ttgatggcaa |
| | 13921 tagagggtat atcaggcagg ttatattggg gcgcggtcaa agagattatt gcaggaaagg |
| | 13981 ttgagttttt cggccgtgag acaagagggg cagtggataa ggtaaactct ctcttaaatt |
| | 14041 atggttatgg cattctctat tcgcatgtct ggggagcaat tataaatgca ggtcttgagc |
| | 14101 catttgcggg atttctgcat gtggacaggc ccggcaagcc gtctcttgtt ctggatctga |
| | 14161 ttgaagaatt ccgccagcct gttgttgata aggttgtaat tgcccatatc aacctcggag |
| | 14221 aatccattaa catgaaaaat ggattgattg aagaggaaac aagaaagaca atgggaagca |
| | 14281 aaattataga aaggctggaa agccaggaaa catttgaagg caagaaatat aaaatcaggt |
| | 14341 ctattatcca gatgcaggca agaaacctat cggctttttt aagcggcagg agagaatata |
| | 14401 aggcgttcag gtttaagtgg taattagttc acgctgaaaa atgcccatct gctgcgttgc |
| | 14461 cttcgtcgct gcgctgctca catacctaaa agcgtatgct ccgctgctcg ctcctcggcg |
| | 14521 ccttgcatct gtgcaatttt tgagcgtgaa ctatataaaa gcatttttca actaattatg |
| | 14581 ggagaacata ctgtttatat tttctatgac atagaagacg acggcatccg caataaggtg |
| | 14641 gcagagacct gcaaagacta tgggcttcaa aggattcaat ttagcggatt ttcaggcacg |
| | 14701 ttgggcaaaa ataagcgcga agaacttttt ttaaagcttt cctttctcat tggcgataaa |
| | 14761 gccggaaaac tccttatgct ccctgtttgt gaaaaagaca tcaatgccaa aagggaactc |
| | 14821 atacaggaaa atagagatga ttcatctcag ggttagcgac cttaaacagt ttgtctattg |
| | 14881 tcaaagggtt gtgttctata actatgttat gcctgttgag aaaaaggcaa cctttaagat |
| | 14941 ggaatatgga aagatagcag aggataagat agacagatta gaaaacagaa ggaaacttaa |
| | 15001 ggaatacggc ttatccagcg gcacgagaga atttcataag caattatatt ctgagagata |
| | 15061 tgctttatcc ggcaaggtgg atttgctaat taaaaccaaa gattcttatt atcctgtgga |
| | 15121 ttttaaatat acaacctctc agccgcacaa aaatcatctt tatcagcttt tgggttatgc |
| | 15181 tataattttg gaagatgtat ataatcgcaa tgttgataag ggttttgtat acttaattcc |
| | 15241 aaaagaggat gctgtggttt ttgatttaac cggcaaagtc aaagaggaaa caaaaaattt |
| | 15301 attagacgat atccgaaaaa tgatacactg ccagcaaatt ccgcctccgg taaattctcc |
| | 15361 ggccaaatgt cttgactgcg aatatcgtaa tttttgcgga gatgtattat aatccgaaat |
| | 15421 agtattatgc ccctttcaaa agaagataaa ttgctcctct taaaaatagt aagggagacg |
| | 15481 attgaatctt atattaagac taaaaagatt cctcctttca atgtgagcag cccggcgctt |
| | 15541 ctggaacatc gtggggcatt tgtttctata aagacaaagg gcaatttgag ggggtgtata |
| | 15601 ggcatctttg cgtcagaaaa acccctttat ctcactgttg tagacatggc tgttgccact |
| | 15661 gctacgcagg accccagatt tattccgctt actgcaactg aactatcact gattaccata |
| | 15721 gagatatctt gtcttacggc gctaaaaaag gtaaaagata ctactgaaat agaggttggg |
| | 15781 agggatggtc tgtatattgt aaaggggtat tgcagaggcg ttcttctccc gcaggtcgca |
| | 15841 gtagagtgtt gctgggacaa agaaacattt ttagaacata cttgccttaa ggcaggtctt |
| | 15901 ccatctgacg gctggaagga tggggctgat atatatacat ttgaggcaga ggtgtttggc |
| | 15961 gaaggcgatt gataaggccg taaaatagcg attaaaaact tcgctatttt acaaacctgc |
| | 16021 gatttgcctg ctccccgata gagacactcg gggacaggca tacttcgtta tcgacacgct |
| | 16081 cagcgtgcct tgaccaagct tagcttggtc ggcgcatttt tgctcctcac cgtatcctcc |
| | 16141 ccctgtcccc ctgtggggac agatttcaaa tctgtcccca gaaaaggggg agggctcgtc |
| | 16201 gcaaaaacat gtccctgagt gcttttatca gggatggcct ctgcctcgtc tgacaacccc |
| | 16261 cgatagaatc aatcgggggc ggtttcccca aaaatcaaac gctatttttg ggtaaggtat |
| | 16321 ttttagtgaa aaatattctt actttatgct aaaataaaac cccaaatttg tatacgaatg |
| | 16381 ttaaaaaata attctacaac aatccatgag actgattaaa tctatactta ttattttaat |
| | 16441 tatctgttta ataacttcca tttttaggac aaatgcagag gctataccag cctttgcaag |
| | 16501 aaagtataag acgagctgca caacatgtca tgcagctttt ccaaaactta atgcctttgg |
| | 16561 tgaggcgttc agaagaaacg gataccagat cccagaggtt gacgaacgct atgttaagga |
| | 16621 gaaacctgtc agcttgggcg ctccggcgtg gaaagaggtt tggcctgaag gaatctggcc |
| | 16681 aggcgagatt cctggggcaa tacccctttc tttatttgga agtttgtttt accgatatga |
| | 16741 tgtaaactct cgggtaaagc atgattttac ctttccaagt accatagcgc ttgaatcagc |
| | 16801 tggaacactc ggtgaagatg tatctttttt tggcagtgtt tctcttatta gaagtggaaa |
| | 16861 tgagttcggc ggtttgtggc gttttttcct gaagtttaat gatacattca gcagctcgct |
| | 16921 tcctgaccgc cttttgaatg tgactatcgg tcaattcgaa ccgtcagcag tcccggtctc |
| | 16981 caataatcgc cgtttaacat atgcgccata tctcattaat acgtttacag tcgggaaaaa |
| | 17041 cagctttaac ttctcaaatc agcgaggcat agagatagat ggcattatag agagccgctt |
| | 17101 agaatatgca gtgggcatag ttaatggcaa cggtacaggg aagacagatt ccacaaccaa |
| | 17161 ctctgcagac aataatacga caaaggacgc ctgtcttaag gttggatata agttcggcgg |
| | 17221 cattggcctg gatggttctg gccttatctc agacaaagaa gatgtcctgc cagaggtaaa |
| | 17281 ggatgaaaaa tcgctatatg ttggcgtaat aggttattca ggcaaaaata aagtggttgt |
| | 17341 ttcaaacagc acgtatgacg ataatttcta taggtatggc tttacctttg acttcaatta |
| | 17401 taaaattttt aatctgtttg gcgcggttat tcagggggag catagtaatc cgagtggcaa |
| | 17461 ttttagtgat acctctgtaa gttcctattt ttctgaagcc gaccttgcca tttatccttg |
| | 17521 gcttattgca gcggtgaggt atggagtagc tgatatagaa catgagaagg aaaataaaga |
| | 17581 tgaagtggtt gtaagcttgt tggcgctggt tcgcgcaaat ataaagctta ctatcgaagg |
| | 17641 ggcgctgcat accacaggtg gcgaggggga cctgggcata gttaaacttg attttgtctt |
| | 17701 ttaagccaac attttcttat gccaattcgc tctgcgagaa tgccgtgtaa accccgttag |
| | 17761 agaactatgt ttcctaacga ccaaagatag tggcagcgcc caccgtaaga cctctggtct |
| | 17821 cttacggggt gaactaccgt ctctaaggaa tgatttatac taccgtccgc ctcaggcgga |
| | 17881 ggagtcggag gctttctcta acggggtaaa atatacttgt agcgccatac aatgacaaca |
| | 17941 cctcaatttt tcaataatac tttctcatag acctggggga agcatctatg attttaatag |
| | 18001 acataagcaa gaaactgttt aactcaaaaa atgcagttgc attttttgac aggagtggta |
| | 18061 agtatgcgat tgtgccgttg cagatgaaag cgcttctgcg gttgagatat ttagccgcat |
| | 18121 gcgcaatagt gcttattttg agctgcatat cgcatgccga gactattaga ggaaccgtca |
| | 18181 agctaaagag tgaagatgat gcggggaaag tagttattta tgtcgaaaaa gctccagggg |
| | 18241 gatttatacc gccagagaag catccggtta tggaccagat tgaactgata tttgttccgt |
| | 18301 atgttcttcc tattctcata ggaactactg ttgattttca taatagcgat gatgtgctca |
| | 18361 ataatatctt tactccgtca tgggcagggc acaaatttaa tctcggcacc tatcccaagg |
| | 18421 gagtagtgag atcatttaca tttgaccgtc tcggagaggt agcattatta tgcaacatcc |
| | 18481 atcctgatat ggagggctat atactggttt tgcagaatcc gtattttgct atccctgaca |
| | 18541 agcaggggag gtatgagata aaggatgcgc ctccgggggt ttatgatttc aaaatgtggt |
| | 18601 ataagaggaa agtaagccct tcttatacgg tgactgttga aaaggggaaa gatgctatag |
| | 18661 tggattttaa ataagggtaa cgggaggtaa cgacgttgaa ggtgacaata agggcaaaat |
| | 18721 taatgattgg gtatatagca gtcctaatct ttatgattat ggtgagtatc ttcgcatata |
| | 18781 tcacacttgt taatgtcaac aaaacagtaa atcaaatact tgtttctgcc cataaatatg |
| | 18841 acacggttaa cagcttgcgg cagtctgtta aacaatttgt tgatgtcagt gattcgttag |
| | 18901 taagagggca gattcaggat gtgggttatt atcgctctct aacgcttgat gtgaaaaaaa |
| | 18961 agcttttgta tgtccgtaaa ctgcgactca aggagcatga aaaggatttc ttggaaaaag |
| | 19021 tagatgctga atttgactct atacaaaatc taacagagca atccttaaag tggacagcag |
| | 19081 ctttgcggaa cacaaatctc gttggcgtgc tcaaggaatt ggacaaggcc aagccagtta |
| | 19141 taatcagcag tgttgatgga ctgtatgatg aggcatggcg ctcactggat aacgttacta |
| | 19201 ttatgactaa cgataatatg aggaagggcc tgcaacagat tctgacattt tcgataatcg |
| | 19261 ctattttggc gggaatagga atatctattt atatttctca aaagattact acgccaataa |
| | 19321 gggcattatc catgtccgct gccagtgttg cgaaaggcga tttggataaa atggtggaga |
| | 19381 agtcgtctgc agatgaaatc ggggaattag ttgcttcgtt caatcagatg ctggttgaat |
| | 19441 taaaaagctc aagggaacag attgaaaagt ataacaaaga gcttgagacc atggtggatg |
| | 19501 aaaggacagc agagctggaa aagacaaagg aataccttga aaatattctt gaacattcgg |
| | 19561 gagatatgat tatcacaaca accctctatg atgagatagt gcagtttaac agaggggctg |
| | 19621 aaaacatact gggctatgtc aaggaagacc ttggcggaat caagatagga gatatttttg |
| | 19681 tcaataagag tgagtataag cggataagaa agaaggtaat cgaggacgga gagatatccg |
| | 19741 gttatgatgc gcagcttatg aagaaaagcg gtgatatcat acgcgtaagt cttacgctct |
| | 19801 cgcgtcttga ggacaggact ggcaatatta tcggtcttgt aggcataggc aggaatatag |
| | 19861 gcgaggataa aaaaagtatt taaatgaaga tacataaaaa ggggattcaa cgcaggatag |
| | 19921 ccatttttat atttgtcgta gggatgtttc ctgtcatgat aagcatactt ttagtttatc |
| | 19981 tgcagggggt gagggagctt agagtaagtg cgggtgaaaa ttttgcgcga gtagctaaag |
| | 20041 agatagcgag taacgccgaa attgttatgg agcaatctgt tcacgatgtt aaaagccttg |
| | 20101 ctatttcacc ggtcttaaga aatgcagcga tattagcaaa tgaatcctat acaggtaaag |
| | 20161 ataaggggtc aatagagagg catatccaga aaattgatat gcagtggttg cagcatggag |
| | 20221 acaaggaaaa acgttttggg gcgtatgtat ccagtcgggc tgcgcaatac ttagcagaaa |
| | 20281 taaagaaaaa tacagaggga tacgttgagc tttttataac cgacacaaaa ggggtattag |
| | 20341 ttgcctccgc aggaaagaca aagtatttat actatggcag tgagaagtgg tggcagacag |
| | 20401 cctataataa aggtaaagga gatatttaca taagcgagat ataccttgac cctgatatca |
| | 20461 atcagttcct acaaagtgtt gcagtgccaa ttatgtctga gaaagaagga aaggtgctgg |
| | 20521 gagttattcg tgcagttagt accatagata agatttcgaa gatagtgcag aaattcagaa |
| | 20581 ttggaaaaac aggccatgca atgcttataa attccgttgg gattatcctc ctgtgtccta |
| | 20641 tattcccgcc tcaggttcac agggttactg acgagctgat gaataatatt accacagcgc |
| | 20701 cgttacttgg ctgggcggtt gttaaggata atgcccatga aggcattaat gctgttgcgg |
| | 20761 gatttgcccc tattgcaacg accgatattg taaagaatgc ctttgatgga aacaaatggt |
| | 20821 atatctttgt cagtcaatca ccggatgaaa gttatgcccc tatttatgtt ttgctaaaga |
| | 20881 gggttttttt tctactgata ttctctattt taatactctc tgctatggga ttctgggcgg |
| | 20941 caagaaagat tgtgaagccg atagagacgc ttcggcaagg ggttgagata attggggaag |
| | 21001 gcaatttaaa ccaccgtatc catattaaga cacatgatga gatagaggag cttgcaaata |
| | 21061 aattcaatca gatggtggaa aaaattaaca agacacattc tgaacttgaa cagcgtattt |
| | 21121 cggagaggac agggtttttg agaaaaggct atcaagagat ggaagtaatg agccgtctta |
| | 21181 agtcagaatt tctcacaaat gtatctcacg agctgaggac gccgcttaat tccattttgg |
| | 21241 gattttcaga gctgctccac gataaggttt gtggagatct gaatgaaaag caatctgaat |
| | 21301 atatagggta tatttatcgg agcggtaaac atcttctcga tataataaat gacatccttg |
| | 21361 acctgtcaaa gattgaggca ggaaagatgg agataaaacc agaagatttt ttagtttcca |
| | 21421 atggaataaa agaggcatgc gctatagtga ctcctttggc tataaagaag aaaataccta |
| | 21481 ttgatgttga aatagcacct gatgtttata ctatcgtggc tgatataagg atgtttaagc |
| | 21541 agataatgta taatcttatc agcaatgctg tgaaatttac ccaggaaggc ggccatgtca |
| | 21601 gcattaaggt taagtccaac aactattatc ttcaggtttc ggtgattgat actggtgtag |
| | 21661 gtatcaaaag ggaagatatg ggcagcatat tcaaagaatt taaacaggtc tcaaccggag |
| | 21721 atcaggaggg aactggtctt ggtcttgtcc ttgtaaaaag gtatgtagaa atgcagggcg |
| | 21781 gtagtatcag ggttgagagc gaatttggca aggggagtaa cttcaccttc aggcttccga |
| | 21841 tgaatattac gaaaatataa agagcaggct atgggcaatg ggctataggc tatggggttt |
| | 21901 ttcctattgc ctatcgcctc taccctactg cctactttta ataggcatat aaatttccat |
| | 21961 atggtcggag ggaggcaggt gtcagcttct taaaaccgga tttcattata tcttcggttt |
| | 22021 taaggttaaa gtctttgcaa taatcctcta aatgttcctt gagaaatttt ttgtcctctt |
| | 22081 cgtttaaatc tgtaattcca acctcagcgc cgcacccttt tttatccaca tcgcctctta |
| | 22141 taaatatcac gccgccatgc atgcctgtgc caaggtaatc cccaaccaac ggccttttat |
| | 22201 tgtcgccatc aaggcccagg aggattaata cgccgcctgc catg (SEQ ID NO: 40) |
| 0209542100 | nnnnnnnnng cttgcaatga |
| 61519_ | 9661 aatttaatgc tacggatact aacacagaac tattgaacat tacaaacctt aaacccctta |
| organized | 9721 agcccgggcc gtacattttc cggcaaattg cggccttgca cagcggatat aaaacgaaaa |
| Ga0209542_ | 9781 aactttactg cctttcttgc tttcaatcat ggaagttgtt ataaagatgg ttgcgaagat |
| 10061519\| | 9841 ggcaggaatg aaagaaggta gcaggcaggg aagggtgtgg tatgatggct gcaaatttga |
| GENOME: | 9901 caatttctaa ttttaataga accgttgtcc cattaaccat cttgctaaaa accgatggga |
| RifCSPhiglS | 9961 gaggaaagag agtgtttgag gtttagcatg aggaggtttt atggaaagag aaattgtttt |
| PAdes_$F_33 | 10021 aggcattgat tacggtggca aatataccgg tcttgcggtt gttgaccgaa ggcataatca |
| 00025323 | 10081 ggtattatat gcaaacagag tgaagatgcg ggacgatgtc gctgatattc tcaaaggccg |
| | 10141 cagagagcaa agaggtatca gaagaaccgc tcaaaccaag aagaaacgtt tgagagaact |
| | 10201 cagaaactat ctaaaaagta ttggatataa cgaatccaca gcaacattta aaacggttta |
| | 10261 cagccttgcc cataagagag gatacgatta tgctgatatg cctgaagaaa agacttctga |
| | 10321 aaagatagag gcaatggatg aggaagaacg gaaacagtgg gaaaaagaga aacaggaatg |
| | 10381 ggaagagaca aaaagaaatt caaggcacag ggaagaagtt ttaaaagatg tccgtaaagc |
| | 10441 aatgactgaa ggaagagcag ccgaggagca gataaaaagg gttgaaagta tttttaataa |
| | 10501 acagtacagg ccaaagagat tcaacaacag aattttgacc aagtgcaagg tctgcggaaa |
| | 10561 aaatacgcca ttaagaagaa aagtgaggga gttgcttctt gaaaatatag ttcgcttcct |
| | 10621 gccgttagaa agcgaaatga aagagactct taaacagaca attcttgaag gacagcagga |
| | 10681 aaatatcaat aaaatattca ggaaattgaa atttaatcag aaagactggc cgggcaagaa |
| | 10741 tcttacggac atagcaaaga acaaactgcc gggcagactg cctttttgca aggaacattt |
| | 10801 ctccgagaat gagaagttca ccatgattga aaaatcaact ttccgtcttg caccatcctt |
| | 10861 aaaaacaaaa atagagaatg tccttgctgt tattaaagac gagatattgc ccaaatttac |
| | 10921 tatcaataag gtggtaatgg agagcaacaa ttttgatata gccgccaaaa cacagggaaa |
| | 10981 aaagcgattg gcaaaggaag aatatggcaa agggcccagg gaaggcaaag agaccagaaa |
| | 11041 ggaagccctt cttcgagaaa cgaatggtag atgtatctac tgtggaaagt ctattgacat |
| | 11101 atccaatgcg catgatgacc atatatttcc ccgaaagtcc gggggcttaa atatctttgc |
| | 11161 aaatttagta gcatgttgcg ctgtatgcaa tgagaataaa aaagggagaa ctccgttaga |
| | 11221 aagtggaatc ctgcctaaac ccgacgttat acgttttatg gaggatgaat ttaacagact |
| | 11281 taaaattaag atgtggaaga tcaggcgaaa gcagtcattg acaactgaag agattatgga |
| | 11341 taaggcaaag cgcaataagc ttgaactgaa actaaaaatc cttgaagatg cgcagcaggt |
| | 11401 caatgaactc gatttcaaca agtatatgtc tcatgcctct atcggctgga gatatatgag |
| | 11461 ggacagactg agagagtctg ccggcaataa aaagttgcct atagagaggc aaagcggcat |
| | 11521 atacactgca tatttcagaa gatggtgggg atttaaaaag gaaagaggca acacccttca |
| | 11581 tcatgccctt gatgctgtaa tccttgcttc aaggaaaggg tatagtgatg atggattggt |
| | 11641 ggatatgacc cttaagccaa aatataacaa aggtggagag tttgaccctg aaaaacacat |
| | 11701 tcctgaaccg atagaattta aaagggataa aggcagcaag ggctctgcat tgcatgatag |
| | 11761 aaacccactg tcttataaga agggcattat taccagaaga tttatggtta cagagattga |
| | 11821 gtgcggtaaa gaagacgatg ttatttccga gacatatcgt gaaaagctaa aagaggcttt |
| | 11881 taaattattt gatacgaaaa aagggaaatg tttaacagac aaggaggcaa aggaagcagg |
| | 11941 tttttgcatt aagaagcatg aattggttat gagccttaaa tgcagcatta aaggaaccgg |
| | 12001 gccagggcag atgataagaa tcaataataa tgtttttaag acgaatgtcc ataatgttgg |
| | 12061 cgtggatgtt tatcttgatg aaaaaggtaa gaaaaaggca tatgagcgaa agaatccgag |
| | 12121 actgtcaaaa cacttcatag aaccaccacc gcagcctaat gggagagttt catttacctt |
| | 12181 aaaacgaaga gatatggtta cagttgaagg tgaagacgca atctatagaa taaaaaaact |
| | 12241 cggaacatct cctacaatag aagcagttgt tggtagtgat ggaaaaacaa gaactgtctc |
| | 12301 tgccaccaag ctgacaaaag caaacaatgc tgaatagttt ttaaaattaa atgtcctaac |
| | 12361 ccacgatgaa ggcgccattt ctggcataga ggctcaggct ggacaaaccc aagcgtatat |
| | 12421 atcttctcgt ataggcgttt tacaattaaa gctcttttga atttcgcaaa cctctatggg |
| | 12481 tttctttcaa aaaatatctt ttcggaaatt ttgcgatttg cttctgaaaa accctttgaa |
| | 12541 actccagcaa aacaaaagcg gtttcacgcc atattttcaa agagccttaa ctgtaacaaa |
| | 12601 gtaagagtgc cggttgattt gaaattcgtg ccaaagtttc acgccatatt ttcaaagagc |
| | 12661 cttaactgta actatacata ttatattcaa tttcctgcaa ccattctgtt tcacgccata |
| | 12721 ttttcaaaga gccttaactg taacctttga ttgtctcaat cagggtatcc aatttaaggg |
| | 12781 tttcacgcca tattttcaaa gagccttaac tgtaacaata agaagccgtc attgctgcaa |
| | 12841 gcgtgccgtc gccgtttcac gccatatttt caaagagcct taactgtaac atgtattgcc |
| | 12901 tgcaacattc tggatgattc caaattcgtt tcacgccata ttttcaaaga gccttaactg |
| | 12961 taacttcttt ggttcgggtg cggtgttgtt tgcaaggcgt ttcacgccat attttcaaag |
| | 13021 agccttaact gtaacttcat aataccatac taaaacgaaa ctcatgctaa tgatacttcg |
| | 13081 gctgtcttgc ctgtagattc attaataaca aggagtttac cctcatcctc ttttttcaac |
| | 13141 tgttcattgg cattcagcaa ttctatgcca tagaccgttc catcaggcgc caaatctatg |
| | 13201 ttcatttcat cgctgatttt cacagtctcg acttgcgctg gtttttcatg caggcgaata |
| | 13261 taagctatgt tgtatctcgg atcatatgtt aacttcatac tatacccaaa ctctgccgcg |
| | 13321 cctctttccg ccaatcgttc ctatgcgtgt atatgaaatt ttaccatata cggcttctat |
| | 13381 caatatgtcg gaactgttgc aatcgcaaga cttgcaatat gatttgcact gctttatagt |
| | 13441 tacaccacat aacctcaaaa atcaagaaaa tattaaggag ggcatatggg taatgataat |
| | 13501 tcaaaaatcc tccgcattca tccggaatac caaccagcag aaaaactccc gcctgcacag |
| | 13561 ttcaatctct ttacaggaga agcagagcca attccacaac tgattgacaa aagtattgat |
| | 13621 gacgaaatat tcattgacaa tgggatttcg cttgtgcaga gcggagattc ctcacaaata |
| | 13681 gttctgtcag gttttggcat ctttttaagc aaaaagagcg aacagcttat cgtaaagaag |
| | 13741 agcgacaagg tcatttatga attttccctt tgacaaactt ttttctacct gcaataattg |
| | 13801 gccttgcccc aaaaatagac attgattttt ggggaaactg acaagttgcc tgccagacga |
| | 13861 ggcggcggcc atttttgtga cgcaaccgta tgctcaccga tacggtgagg aacaaaaatt |
| | 13921 ggccgataac gaagtatggc aggtaaatcg ccagtttgta aaatagcggg ggttttaatc |
| | 13981 gctattttac ggcttgtttt taaaattcta ttgtattgca agcttataga tgatctcaga |
| | 14041 aaacaaaaga ctcaaaaaca cacccctgca tcaaacccat aaagaactca atgcgaggct |
| | 14101 ggttcctttt gcaggatggg aaatgcctgt gcagtacatg ggcgttatag aggaacatct |
| | 14161 taatgtccgc aaggcctgcg ggctttttga tgtaagccac atgggtgaga tagagatatc |
| | 14221 cggccctaag gcccttga (SEQ ID NO: 41) |
| MHEFO10001 | nnnnnnnnnn nnnctggtta aaactacctc |
| 35.1_ | 8281 atgtttatta taacaaattt gtttattttg tgttgaagta tccactgcac aggaagaaaa |
| organized | 8341 ggggaggtgt taatgctcac taattaggta actttttgga gtttagcagt tcatcatatt |
| Nitrospirae | 8401 taccatcgag ataagtgctt gaaaaatatg gtaggcacga ggggtatcga acccctgacc |
| bacterium | 8461 tcttccgtgt caaggagagt ctgtcgtatg tcgaggtttt acgaatcttt ataaaccttt |
| RBG_13_39 | 8521 gtaaattttt gttgcaaagg atttgggggg agaagggttt tatatccttt tcccttcttt |
| 12, | 8581 tttgactgtc tactatacaa aaactataca atggcattaa acaatactta tttttcagac |
| RBG_13_seat | 8641 ccccctacat cccctttttc agaagagggg tgggggttaa gtgcttaccc cctttctcgc |
| fold_5832, | 8701 atatgaaatc agttttatgg gaaaggctaa aattgaccta atagacgctg tctaatttaa |
| whole genome | 8761 aggttcactt tgaggatttc acctacaact attcatcttc agcaagttca aagagttaac |
| shotgun | 8821 tatttccgag agaaattaac aactattctt ttgtaaactc atcaataaat ttatcaacat |
| sequence | 8881 tatgtgtctt acgctgtatc aacttaaata aacgccaaag ttcccctggg gctgataact |
| | 8941 cttcaaagtc aaattcatat tccaagataa aatctcttga cgctaatgca agctgtggac |
| | 9001 ttgaacttcc ctccttaata taaattacta tagatttgtt accaaattca caataataac |
| | 9061 catctttcag tgtcgagata tcatcacgaa tgaactgata aataatattt ggagataggt |
| | 9121 cattaataag actattccaa tctaatttat ttgcttttgt tttttcaatt aagctccgaa |
| | 9181 gaaaggtcat ttctttcata ataaaaattc ctctttttta gaaagtttgc caagcaaaat |
| | 9241 agctacctta tctgcgactg ctattgtatc aatattgtct aaatcaccat ctaataatcg |
| | 9301 ttttagtttc tccagttccc ttttatgttc tttcccaaat tgaaattctt ccaatgtgct |
| | 9361 tatgagtctt tttaaatcgg aaatctgttt tctatcaaaa atattatctt catttataag |
| | 9421 ccgtagacac ccatctaact ctgcataaat tttatttttt tttgaacccc attgtttata |
| | 9481 atcaatgtat tttattaaag ttttctttac cccatgagca tatattaata atataatgct |
| | 9541 aactactaag ctaagaacac ttgcccaatt agctatatag tcaaacatct ccattccatt |
| | 9601 agttaacgca ttcaatatat atgacagtta ggaaatagac acttgattac cgatacaaat |
| | 9661 ctacaattta ttcataattc ctgtcaatgt taacttcagg aagaagcaaa ccggaaactt |
| | 9721 ttcttcctga aggtgacacc ctgaataatt cgcaaaatgt gagtagtact cttggtatta |
| | 9781 aacagttgtt aaggtggaaa caatgcgaag tatgtgtaga acaaaacaag gataatgttt |
| | 9841 tataagaaaa gaagtgcgat atcatccaga tgttaatctt tttcgccttt tacttgctgc |
| | 9901 catgaatttt gattctaaaa aatgactttt ctaacaaata attccttgta tttttaattg |
| | 9961 gaaagttgta aaaaaatctt atactttaac ttgacttatt atggaaaaag aacttgtttt |
| | 10021 aggtattgat tacggtggca aatatactgg tcttgcggtt gttaaccaga aaaataatca |
| | 10081 agtactttat gcacgaacag taaagatgcg tgatgatgtc acagacattc ttgcaggtcg |
| | 10141 cagagaacaa agaagtttaa gaagaacact acaaactaag aaaaaacgtt taagagagct |
| | 10201 caagaactat cttgaaagta ttggtggtat atatgaggaa tcaagtggga catttacaat |
| | 10261 tgaacctttt agaacagttt acagtcttgc ccataagagg ggttatgatt atgccgacct |
| | 10321 ccctgaagaa aaaacttctg aagagataga ggcaatggat gctaaagaac gaaaacagtg |
| | 10381 ggaaaaagag aaaaaagaat tggaggaaac gcaaaggaat tcaagacata gagacgaagt |
| | 10441 tcttagagat gtacgcaacg taatgactga aggtaatcta tcagaagagc agataataaa |
| | 10501 ggttgaatct attttcaata agcagtaccg ccataaaagg tttaataacc gaatcctgac |
| | 10561 taagtgcaaa gtctgcggta aaaatacccc attaagaata aatgttcggg aattgctact |
| | 10621 tgaaaatatt gtgcgttatt tgcctctcca gaataaggaa agagaattgc ttaaattgac |
| | 10681 tatcttgaaa gggcatcagc aggatataaa tgaaatcttt aagcacttcc gaaaagttta |
| | 10741 caagataact ctaaaccaaa aagattggcc agggaagaat cttattgata tagcacggaa |
| | 10801 tcaattgcgg ggcagattac ttttctgtaa agtacatttt cccgaaaatg aaaaatatgt |
| | 10861 ttctattgaa aaaaagacct tcaggcttgc accatcctta aaaacaaaga tagaaaatgt |
| | 10921 tctgtcggtt atcaaagatg acattttgcc taatttcact ttaaacaacg ttgttatgga |
| | 10981 aagtaataac tttgatatag cagctaagac aaagggaaag aaacgattac ttaaagaaga |
| | 11041 atacagtaaa ggacacagag agagtggaga aaccagaaaa gaagctcttc ttagagaaac |
| | 11101 agatagccga tgtatttact gtgggaaagg aatagattta tctaatgctc atgaagacca |
| | 11161 tatttttccg agaaaggctg gaggtattaa tatctttggc aatttagtcg catgttgcag |
| | 11221 cgtatgcaat gaggaaaaaa gaggaaggac accattagaa agtggcattt tgccaaaacc |
| | 11281 agaaattgta tctttcataa ccaatgactt gaagaaaaag attcttgaag atgcacaata |
| | 11341 tattaatact ttagatttca acaaatacat gtctcatgcc tctattggat ggcgacatat |
| | 11401 gagagatagg cttagagaat taactggcaa taaagaatta cttataaagc gacaaagtgg |
| | 11461 tatctatact gcatacttta gaaagtggtg gggatttatc aaagaaaggg gcaaccacgg |
| | 11521 acatcatgca cttgatgctg taattcttgc ttcaaagaag agctatgcag aagatggcaa |
| | 11581 agtagatatg actattaaac catgtggcga agatggcaaa gaatttgaca tagaaagaca |
| | 11641 cctttctgag atgaaagagt tcagaagaga taaagggggt aaaagtgctc ctcttcacga |
| | 11701 taggaatcct ttatctttta agaacgatat tataactcgc agatttatgg taactgaaat |
| | 11761 tgagtgcggg aaagaagccg ttattatttc ggaggaatat cgtaaaaaac taactgaggc |
| | 11821 ttttaaacgc tttggtattg ctaaagggaa atatttgaca gatgaacagg caaaggatgc |
| | 11881 tggattctat ttaaggaaaa atggcgaagg ggtgatgagt cttaaatgcg aggtaaaagg |
| | 11941 aactggctat aatcagatga ttagaataaa aaataatatt tttaaaacaa atgttcataa |
| | 12001 tgttggcgtg gcagtttttc ttgatgaaaa aggcaaaaaa agagcttgtg aactcaagaa |
| | 12061 cccaagactg tcaaagcatt ttgtaaagcc agcagagcag gttaaaggaa aagttatatt |
| | 12121 catcttgaaa cgtggtaata tggttacagt tgaaggtgaa gaaatgatat atagggttaa |
| | 12181 gaaacttggt acttctcctg ttatagaagc cattgtcggt agtgatggaa aaacaaggac |
| | 12241 tgtatcagct actaaactct tgaaaataaa ccatactaaa aaggtttaat taaattgtcc |
| | 12301 taacccacaa tgaaggtgcc atttctggcg tagaggttta ggctggacaa acccaaacgt |
| | 12361 acataccctc accgtatagg cgttttacaa ttaaggctct tttaattttc gcaaaccatt |
| | 12421 aaggatttat ttcaaaaaac aacttttcta aacttttatg aattatcact gacaaaacct |
| | 12481 ttgaattaaa atgaaaataa ttccgttaca gtcgttgttt tcaaagagcc ttaactgtaa |
| | 12541 ccccatatat cagttataaa cactatattc tgccatgtta cagtcgttat tttcaaagag |
| | 12601 ccttaactgt aacctaccag tgagccgatt cataaggcga acagtaaggc gttacagtcg |
| | 12661 ttgttttcaa agagccttaa ctgtaacctc ctgatgatgt acctcctttt cgacagggcg |
| | 12721 ggatttacag tcgttgtttt caaagagcct taactgaatt tgcaaacata attaaagtgt |
| | 12781 aatatgatta caccatattg cctaaaaatc aatagtatta taatagaata aactattata |
| | 12841 taataagtca atataaatcc taaattgtat agtattttca aactgttatg gggttcaaag |
| | 12901 aattatgagt ttattaaaat atggctattt gggcatgttt tttcggggaa agggggtctc |
| | 12961 acgtacacga aaagctaatt tttcagaaaa gcgtaaaaga tagctttccc cataactgag |
| | 13021 acaaaagaga gattgatata cttttggatt tgtgcatata atggcaaaag tgggggattc |
| | 13081 ataactatga tacatgttgc tccatctgat tgcagatagt taaatgataa cttctaatac |
| | 13141 atgctatcca tttggtggga aagtcgcaac agtcgaaaca ccatcattta aatgcatgat |
| | 13201 tagtacagcc aaagaaggcg ttttcaagaa acagtccgta gtttttcttg atattaatat |
| | 13261 gggcatggct aatcgccgac ctgtcgctaa gctgcattac aatgacatac tcactaaaga |
| | 13321 acaacgtttg caatttcacg atatcatagt aagcgtgata gatgaatccg gcatgaatgg |
| | 13381 actatctctc tatgatacac tcagggactt gtttcaagga cttcgcaaag agggcattgg |
| | 13441 ttctgggttt tttacagata cccattgatg acggctgaga agtgaaaagg gaataactta |
| | 13501 aaacaattgt ggagacatac ttttcaaaaa atccccttcc ctttaccttt gaaatagtct |
| | 13561 gttgagataa agtgaaagaa gaaaaaataa gggaagacgt tagggattat ttaaactctt |
| | 13621 ttctcattga aggtggttat tgttttaatg accctaaaaa attgtgttta gccattacca |
| | 13681 gtttgtcgaa cgacatatat atcttcagca aaaaagattt ccttgtgcat ttgttaagat |
| | 13741 tcagaactga cattgaaaat gctaaaacct tcaaagaagc ctcagattct tttttaactt |
| | 13801 tattaaaacc aatatctaag gagaaacctg ctgattttat tcaagagaca aaaaagcaaa |
| | 13861 ggatgacaag ggaagatgcc tacggaggat atgaaagaaa gagtgataaa tacaagggca |
| | 13921 aaaaaagagg cgatatttgg aatgttatta aaaaggcaga agatatactg actggtaaga |
| | 13981 agataagtga gtgggagcgc atcggaatag gtgaactcga atggttcaag cttttttcaa |
| | 14041 aactctcaag aggaattgct aaaggaattg aacttcacga cttagaagct tacacgggat |
| | 14101 acagtacaaa ggcgatccat aaaatgatag atgaacagtg tgtattaaag gatgtgttgg |
| | 14161 atatctttaa taaaaagatt aaaacagcat tagcaaagca aaaacctgat gacaggactt |
| | 14221 tcttaaacac gatcaaagga gtatggggta aaaaggcagc attggtctta gagaattgta |
| | 14281 cttatgaagg ggaacgaggc cgtaaagtct ctaaggagac tataaaggag aggcataaaa |
| | 14341 ggataagaga agttatttac agtgggaaaa ttgaaaaggt taattcaggg acttactaca |
| | 14401 gtagagactt tagagtattt ctatgcgtaa tggctcgtgt tgtttatcac ctcttgtttg |
| | 14461 aaattccctt tttaggcgac aaattgaaaa gagaagcaat atcagagttt agaaattttg |
| | 14521 caaaaaaata tgatataaaa ctctctaaat tatctgactt ccaatttata gaaagcaaac |
| | 14581 ccacatcaaa aggcttaccc gacataaagc ctcataaata gccattcttc ctaatagcac |
| | 14641 gaaatagatt caattccgtt cctttcagat caatcagaaa tccgacataa taccaatata |
| | 14701 tgtaaaaaaa agatttttgt tccaattttg ttcccgaatc cgaaaaaagg aggtaaaaga |
| | 14761 ataatgggaa aacacacctt tgaagatact tcaatagtag cattcttatc tctcaaaaat |
| | 14821 tacaaggtaa caccacagag aacctatgac ggaaaagttg tctttattgt agaaggcaag |
| | 14881 gatattaaca gggcattgca ggagctttat ggcaattctc aagtaggtgt tttggatttt |
| | 14941 attaagactt taaaagcctt acgctcaagt atctttgcac tcaaagcagg gggtgaaaga |
| | 15001 tgaaatctat acttgatcag aatttaatca ctgtatattc attccctttt gatatatttc |
| | 15061 catcaaagct attgaatgtc attaatgtac taagtgaagc cttacatgtg gagcctgaaa |
| | 15121 ttgtagcaag tgcaatgctt accataataa gtgggacaat aggcaacact gtaagaatat |
| | 15181 cccctaaaca tggttttgaa gtggcaccct ttatatggct gattatcatt gccttaagtg |
| | 15241 gttatggtaa atcccctgtg attcaaaccc ttctaaagca catcaatgaa ttgcaggcaa |
| | 15301 aggcttataa cgaataccaa aagcagtttc aagaatatga aagaagactt agaaaggcta |
| | 15361 aacaagatga aagcatagat ataccagaga agccaaaatt aaaacatcat gttgtatcag |
| | 15421 attgtacggt ggaggcattg gctaatgtgt ttgagaatga tagcagaggg gttattagct |
| | 15481 atcaggatga aatagcaagt ttaattttag gtcttgacca gtacaaagtc aaagggaacg |
| | 15541 atagacagca ttatttggaa ctatttaact gtgatagctg gaagatagac agaaaatcag |
| | 15601 gtgtcaagtt tatccacaac acaggtactt ctattattgg tgggatacag cctaaagtta |
| | 15661 tgcctgacgt cttcaaagta aattcttttg atgatgggtt tctaccccgt ttcttattgc |
| | 15721 tcaatgctga aaacagaccc atgaagttta gcagacaagc aattattgaa gagattatat |
| | 15781 cttactggag agacctatta aattggtgct atgccattcc attagaacat gatgatgatg |
| | 15841 gattcataaa accaaaagtt ttaaccctga gtagcaaagc cttagacata tgggaacaat |
| | 15901 tctataacga ttacggcgat aagatgcctt tcctttcaga aagggcaagg gtctttatcc |
| | 15961 cgaaacttat agcctatcac agtcttaagt ttgctggagt tttacatgtc atagaggcat |
| | 16021 tcagcaaagg tataacatcc ataaacaact taatcgagga tgagacaata catcacgcta |
| | 16081 ttgaattaat aaaattctat gctggacaga ctattaaggc attgaaactg tatgagaaac |
| | 16141 cagaagacac tctaaatgaa tttcagaaga gactcattga aacactacgt agcttgaagg |
| | 16201 gagaggttaa aaacgggaaa ttacagttat caagaattgt tgagctattc aatcatggcg |
| | 16261 tacctgaagg tgtgagtcat actccagaag gaattagcaa tatgttggct gatttggggt |
| | 16321 taactactga aaaaagcacg ggcaattatt catatctttt atgggaatct gagaagatac |
| | 16381 aaaaactttt ttctaaaaca accgtaacta ctgtaactac tgtaactact gtaactgata |
| | 16441 aatcaaattc agaaaacaac aaagttactg aagttacgga tgttaccgtt aattctgatg |
| | 16501 aagaagttat agaccttgaa catgaggagt ttgaaatagt caaatgagat acaaagtctt |
| | 16561 agaaagtttc aagattaaaa cgttaaaaaa ggaaatggaa ctgcaagcag ggcaggttgt |
| | 16621 cactcttcct catgacaagg ctatcattct aatcagtgaa ggtaagatta ctccctttga |
| | 16681 tgatacagct tatagggtct actcggagaa tctacatgct tacctttggg ttgtagaaga |
| | 16741 tgataaggat attgaccgtc tcagagaaca aggcataaaa gaagctatct acacaagaca |
| | 16801 ggaaatagag aaactaaaag atattgataa agactctctc aaggtaatcc acagagttaa |
| | 16861 agaggtcttt gagtcatcaa aggtcgtgga gataaagggc tgtgaagact gagcttgaaa |
| | 16921 cacaggagat tgaggcgata gccgataagg ttatcgagaa aatgaaaccg ttgcttgctg |
| | 16981 gtaatggtaa gtcagaggat gatgttatct ttgatgtaga ggggttaact caatatttga |
| | 17041 aggtatctaa gcaatggatt tacgaaagaa cacacctgaa aaagatagca cacttaaaaa |
| | 17101 tagatgggca attgagattt aggaaaaagg atattgataa atggcttagc tcttataata |
| | 17161 tccccgctat aaacacacct tgaaggattt taaaggctat aaagtaagag gaggccataa |
| | 17221 gattgtatgt ccgataacct atataagaaa cccttaataa taaacaatct acaaaccact |
| | 17281 gtgctttaaa ttgtgcacta aaaataattc ttgactttct aacaatgtta gtatatgatt |
| | 17341 tagatatgca aacctggaca cctgaagaaa tagagcaatt cagaaaagac aacaagctaa |
| | 17401 gcagaagggc tttaggtgag cttttaggtg ttacaggcaa ttgcatatat caatgggaaa |
| | 17461 gggggttgag agagccgagt aaaacaacaa aaatactttt atcaaggatt gagcaagaac |
| | 17521 taaaaggaaa gaaaaaacaa aaaggaaagg taggtgaaaa gaagcatggc aaaaagtaag |
| | 17581 catcggggga tattcctgag aaaccgcatt tactggatta gatatgcagg gcttgatggt |
| | 17641 aagatggttt atgaatcctc aggcaacgaa aagtttaaag atgctgaagc cctgcttatc |
| | 17701 cagcagaagc aatctatcaa agaaggcaag caacctgaga ttaggaaaat ctccaattac |
| | 17761 acattcaaag aactttctga gaaataccaa tcatggatta acggcagaca gaaatcagcc |
| | 17821 agaacaaaag gctatctcat caaacagttg gttgaaaggt tcggtaactt tccgattagg |
| | 17881 agattcaata cggtgattgt ggaacaatta cagacagacc ttatgaatag aggattaaag |
| | 17941 aatagtagct gtaataaggt cttgaatgtt ctcaaacata tgtttacaaa agctgtggaa |
| | 18001 tgggagatgg tagagtctga aacattaaag cgtaccagaa aagtaaagct attaagagac |
| | 18061 gatgggaaaa ggttaaggta tctctctaca gaggagtgtc aagccttaat caatgcctgt |
| | 18121 gataatcacc tgaagcctat tgtagtaaca gcagtgaata caggcatgag aaaaggtgaa |
| | 18181 atactctctc ttaaatggga taatgtagac cttagacatg gcttcattct gttagatgta |
| | 18241 acaaagaatg gagagagaag ggaaatacct attaacgata ctctcaggat aaccttacag |
| | 18301 ggtattacaa ggcggttaga tattccttat gtattctatg accaaaatat agggaaagcc |
| | 18361 tatcaggatg ttaagagaag ctttaaaaca gcattgagaa gaacagggat aagagatttt |
| | 18421 catttccatg acctgagaca tacctttgca tctcatcttg ttatggcagg ggtagattta |
| | 18481 accacagtgt caaggctgtt agggcataaa actttaacta tgaccttgag gtatgctcat |
| | 18541 cttgcacctg ctcacatggt taaagctatg gatatcttgg acaatacctt aaatggtaag |
| | 18601 tttagctcag taggtgaacg tttgcatgat gcaaactata caaaaactat acaatcggca |
| | 18661 gaggcaaggt aagatgaaat ctcctaagtg cttgaaaaat atggtaggca cgaggggtat |
| | 18721 cgaacccctg acctcttccg tgtcaaggaa gcgctctacc actgagctac gcgcctgtaa |
| | 18781 gactttataa aataacattt tacccattac tcctgtcaac cttgagttta tatcttttag |
| | 18841 tctcttttga cagttaatca tctcttttta tatactttc (SEQ ID NO: 42) |
| MHDT01000 | 1 gcttattcag gaacttaacg gcaacacgac aggcgatagg caagaggcta taggcaatgg |
| 042.1_ | 61 gcgaaagaca aaaggcatcg gcgcattgca ggcaaaactt aaagaggcga gggaatccga |
| organized, | 121 gtataaggca aaagaagacg ccttnnnnnn nnnnnnnnnn nnnnnnnnnn nnnnnnnnnn |
| Nitrospirae | 181 nnnnnnnnnn nnnnnnnnnn nnnnnnnnnn nnnnnnnnnn nnnnnnnnnn nnnnnnnnnn |
| bacterium | 241 nnnnnnnnnn nnnnnnnnnn nnnntttaga cgcccttaaa aaggaaaggg agaagggaat |
| GWA2_46_11 | 301 acttacggac atagtggaca gggaaaagag catcagcgcc atagaaggag aggcgataaa |
| gwa2_scaffold | 361 ggcaaagaag aaatacgaac tcgaaaggct ttgctctcct gttgacggca ctgttcacgg |
| 6184, whole | 421 acttgcctca tacactgtgg gaggagtggt aactcctgcc caacccttgg tcacggtagt |
| genome | 481 gcctgaaggg acgcctctta taatagaagc gacggcatta aataaagaca taggattttt |
| shotgun | 541 aaaagtcggg caggaggcgg aggtaaagtt agatacattt ccgtttcaga agtacggcac |
| sequence | 601 cgtaaaaggt aaggtaacgg ccataagccc ggatgccttc gaggatgaaa agttaggacc |
| | 661 ggtgtataag ataaaggtaa ctatcgaaaa attgaagatc tatgcggacg acaggtatat |
| | 721 acccgtagcg cccggaatga gcgtgtcggt agaagtgaag acaaataaaa ggcggataat |
| | 781 agagtttttc ctttcgccga tcgtcaaata cgcgaaggaa agcctgactt tgaggtaaaa |
| | 841 acgacagact tagggcgata ggcgataggt aatgacggat ttaaattggc agcttaggac |
| | 901 taatgtctaa aagtgagtct gaccttaagc ggatgataac taaggagtat aaaatcgaag |
| | 961 atataataaa accgaaaggg aggccgaaga aagatgaaga caagaataaa tagaaccgtc |
| | 1021 ccgttttttg tatcgaggag tattctctcc cccagggaga aagagatttt ggagcgcatg |
| | 1081 agagacggca aaaccgacct cgaaatatcc gccatttata ccatcagcga acacactgta |
| | 1141 aagtttcata taaagcatat cctgcgtaaa ctgcaagctt caaataggac acacgccgta |
| | 1201 ggcatcgcca tgcagaaacg cttgctcgat taattgacat cgaatctacc ccctactcaa |
| | 1261 atgagtagct tacattgggg aaggggaatg tggtatataa aggcctgcat gtcttgcagc |
| | 1321 cgaacttata gggaaggtat gaacgaattc agtacggcgt aaaagagtaa aagattgtgg |
| | 1381 aaattactgc tcaacagaaa tgcaaaaatt cctggaagac tattccgaca gattgcctcg |
| | 1441 actgtcaatg tccgctccga gccgattgca gaggaggatg cacagctgca tcagatgagg |
| | 1501 tatatggttc acaatatcgg attgatccaa tctgtgagtt atcttgttaa aaaaattagg |
| | 1561 ttggtttttc aacgataagg aaagagcatg ccttattgag atcgaataac gtgaggcaag |
| | 1621 atataatctt gcagattact ctaagtggga ggattgtatg cctgacatga aagatttttt |
| | 1681 aaatccgaaa tcgatgttaa cgcccggcat tgcaggcgga ttaacagcca gtgtttcttt |
| | 1741 aacattggca tctgcatttg caataagctt caaatggtcg gcacttggca tcagtttttt |
| | 1801 gctggggttg cttataatga tttccatgcg gaactctatt cccttattgc agcgatttat |
| | 1861 atattgcatc ctaaattcgc tgattgtttt ttctatggcg ttcggcgccg gtaagggcat |
| | 1921 tgattcgcag cctcagctcg atcctaagat tattgacaaa attgtgaata cgcaattatc |
| | 1981 agtagttgag cctcaacccc cttcggtttt agcgttatcc aatgcatatg cgcaacaata |
| | 2041 tcccattaaa tcagataagc ctaaaaaaag cactgctcct tccggaaccg acaaaaaagg |
| | 2101 gacaggcagc aatgaatcaa aggaaaataa tctgaccaag aaagagatgg aacagttaaa |
| | 2161 aacgtatatt caacaacaac agaattatga taagcgatgg tcgtggtaag agaaaagtga |
| | 2221 aattttaaga acagaaaatg gggaggagaa aatgtgtaaa gataagtcag tgagtcattt |
| | 2281 aaacgagtta ggatggaatg tggtaagatt acctaaagaa aatattaatc cgcttttaac |
| | 2341 tctttccaaa tccaatagct atcttgaaac tttaggagaa atctctgatt tcgtgataga |
| | 2401 agatcaaccc aagcccccag aaattgtcca agaccaatca gtagcagaaa tttcaggttt |
| | 2461 ggagactgac aaatttgaat tgggaatagg actaaagttt ttagaaaaat tcctatcttt |
| | 2521 agtgggagta gcgggattag gattagaagc aagctttaag aatgcagaat ccattcaatt |
| | 2581 tgcctatcag aatgtactga cagattttgt ctatccagtg aagattggaa agtacttatt |
| | 2641 gagtgtcagt cctgatgtca gtagcccctt tatggagcat attaatgaag agggagaggc |
| | 2701 ttatattatt acagacacgc ttagatctaa cacattcggt attgttgcct atgatgaaaa |
| | 2761 aggtgtaaaa attgatttgg atatttctgc attaaagcaa cttcttagcg ctactccaca |
| | 2821 tattgaagta tctaaagatg agaagaaagt tgtttctttt aagggggata aatttctgcg |
| | 2881 ttttgccttt aaggcaatcg gcgtatgggt ggagatacgg gacggcaaag cgagatttaa |
| | 2941 actgaataag ccggaaggac ccattgcacc aatgaaggct ttaccgtcca ctttaattag |
| | 3001 tcccaatgaa ccaacacctg taattttcgg tatgaacacc ctaatcagac taaaatgaat |
| | 3061 cataataact aaaaaggaga taaaacatgt cgaatcaaga atcgatcaac agaggtcttg |
| | 3121 acagggcata taatgagttg aaacctgaag atacgataaa atgcgatata gagtctgtgc |
| | 3181 gtatcgttat cttttcagat catcacaggg gagttcgcga tggagctgac gacttccagc |
| | 3241 ggtgtgaagg ggcatattgt gccgcactcg gatattattt agagagaggt tataacctca |
| | 3301 tagttctcgg cgatgcagaa gaattgtggg agtgtaggcc cgcccctgta attagagctt |
| | 3361 atgaaaacac attgaagctt gaggcagaat tttataagaa tagggaagac cgctactgcc |
| | 3421 gtatttatgg aaatcacgat gattactgga gggatacggg tagtattaat ataggtgggg |
| | 3481 aacaaataac ggcaagggat aagctgatga taagcctgca tgataaaaac aataaatcta |
| | 3541 ttggggacat tttttttgtt catggtcatc aagggacttt tgaaagcgac cgctttggga |
| | 3601 agatcagcag gttttttgta agaaacctct ggagaccctt tcagaggatt actcatataa |
| | 3661 agttgactac acctgccaag gatgcagaat tgcgcggcga acacgatacg gcaatgtata |
| | 3721 attgggcttt aaataagaat aatgggcaaa aaaagctttt gctgattgca ggccatacgc |
| | 3781 atttgccggt attcgcatca aaagatcata taggaaagat tagcgatgag cttaaatctc |
| | 3841 ttcagcagca gcttgataat gcgaggacat ccggcgatcc ccatgttgac ggcattgcag |
| | 3901 aacagacagc ggaaaaacgg gctgagcttg aatatcgaaa ggcgcaaaag aatatgaaag |
| | 3961 aggatcagaa aggcattttt atggagaagc catgctattt taattccggc tgttgttcat |
| | 4021 tcagcgatgg agatataaca ggattggaga ttgctgacgg aaaaataaag cttgttaagt |
| | 4081 ggcccaaaga taatccgaga gtagaagttc ttgaggatgc cgacttgaga agcgatattt |
| | 4141 ttggaagctt atagcattgc tgtaccttaa gttttttaag atgacgtttg tgaaaggaga |
| | 4201 cggcaatgat ttacgaatac gagatagacg gcttgacggt gcggacaggc gatatcatct |
| | 4261 gcacttctga cggcgctgcg gataccgata tcaaaggcca gttctggcgg ctgctgggga |
| | 4321 aaataatacc cggcgaggtc gatcacatta ttatttatgt aggaccaaac ggtcgttgcg |
| | 4381 tagaagcggg agcaaaaggc agggtgataa cctttgagat tatggatact acatgggatt |
| | 4441 ttcagaagat gattgcaaaa aggggaataa tagataccct gtatggagca gcatatccgc |
| | 4501 ttcaggggaa gagtctgagc gaaacggaga taacaggaat acgggaagcg gttgccgcgt |
| | 4561 attgcctgag gcaggctgaa ttggagaagc cgtataatat gaatttcctt gattcaacca |
| | 4621 ccgaagatgc atactactgc agccagctcg cttaccttgc atatttcaaa cacggcatcg |
| | 4681 acctgaatac cggcaaggga atacccgaaa taccagggac tgagcgcata atcttcccgc |
| | 4741 aggagatatg gagcgggtgc gggcataaaa aagcgcggta aacaagtttc gctcttgcca |
| | 4801 aaatccatgc ttattttgta gaataagagt ctaaattttt attttcagga gggtttgcgt |
| | 4861 tgaaagaggg aatacatccg aaatataaag aggtaaaggt cgcctgcgcc tgcggagaga |
| | 4921 cttttactac aaagtccaca atggacaata taaagctcga tatctgctcc aagtgccacc |
| | 4981 cgttcttcac cggcaagcag aagatattgg atgccgaggg aagggtagaa aagttcaaaa |
| | 5041 agaaatacgc taagaagtag cgaataaaca tatcggggtt agagcgcggg gtcgtttgcg |
| | 5101 ctctaacctt ttttttatta aggagatata tgcataagat aggcggacag gccgtaatag |
| | 5161 aaggcgttat gatgaaatct cccgaaggct ggagcgtggc cgtaaggggo ccgaagggcg |
| | 5221 acatcagctt taaaaccgta gtaacgaaaa aaccgaacag gtttttcagg ctgccctttg |
| | 5281 taagaggcgt ggctgccctt attcaggcgc tttccatcgg cgttaaggct atcgaatttt |
| | 5341 cgggaaatat cgcgtatcag gaagacgaga aacctatagg cacgctcggc atgggactga |
| | 5401 ccatcggttt cgcgattatt ttagccatag cccttttcaa gttccttccg ctttttctca |
| | 5461 cgactttaat cggaggcgtt tctgaaggcg tttcaaaaag ctcctttatg ttcaacttta |
| | 5521 tcgacgggac gattagggtg ggaatatttc ttttctacat attcgccatc gggctctgga |
| | 5581 aagagatgag gaggatatat cagtatcacg gtgcggagca taaggttatt tacgcttatg |
| | 5641 aagccgggga ggaactcacg gtcgaaaacg cgaagaaata taagccgtat catccgaggt |
| | 5701 gcggaaccag ctttttgctg atagtgatgg ttatcagcat gctcgtattc atgtcgatcc |
| | 5761 cgcagggctg gtctttttcc ggaaagctga tgtcaaggat tgtcctgttg cctgtgatag |
| | 5821 cgggcatttc ttacgagacg ctgaggcttt cggcgagaat gaaggataat ccggtcgtag |
| | 5881 ggctgatcgt ccttcccgga cttttgctcc agaggatgac cgtgcgggag ccggacgaca |
| | 5941 cgcaaataga ggtggcgctg gcggcaatga acgaagtcct gaaattaggc gcggataaag |
| | 6001 aggtttgcaa acagtgttaa aaaagctgct tacgatagaa gaaaaatacg aagagataac |
| | 6061 caattcgctc gtaaatccgg atgtgttatc gaatccccaa ttgtatcaga aatactcgaa |
| | 6121 ggagcagtcg gatctttttc ccattgtcga gaagataaag gagtataaga agcttcttgc |
| | 6181 cgacctcgaa ggtgcggagg aactgcttaa ggccggagac ggcgatttaa gggatcttgc |
| | 6241 tcaggaagag atggaggagc ttaaaaagag gaagcccgtt gtggaagacg aattgaaaat |
| | 6301 aatgctcctc cccaaagatc cgagggatgg aaagaacgta atcctcgaaa tacgggcggg |
| | 6361 aacagggggc gaagaggccg ccttgttcgg cgcggccctt ttcaggatgt attcgaaata |
| | 6421 ctccgaatct aaaagatgga aaatagatat tatagattca agcccgacgg gactcggagg |
| | 6481 cttaaaggaa gttacggcta atattacggg caaaggcgct tacagcaggc ttaaatacga |
| | 6541 aagcggcgtt cacagggtgc agcgggttcc cacgaccgag gcgtccggaa ggatacatac |
| | 6601 ctcggcggca actgtagccg tgctgcccga ggccgaagaa gtggacatta aaatagagga |
| | 6661 gaaggatttg aggatagata ccttctgctc ttcgggcgca ggcgggcaga gcgttaatac |
| | 6721 gacatattcc gccgtaagga taactcatat ccctacaggg cttgtcgttc agtgtcagga |
| | 6781 tgagcgctcc cagttgaaaa accgcgagaa ggccatgaag gtgttacgtt cgaggcttct |
| | 6841 ggaactcgaa atagaaaaga aggaaaaaga gcgtgccgcc gacaggaaga cgcaggtcgg |
| | 6901 aagcggggac aggagcgaga ggataaggac ttataattat ccgcagaacc gtatatccga |
| | 6961 ccacaggata gggcttacgc ttcataagct cgaacaggct cttgaaggaa atctcgatga |
| | 7021 gcttatcgac gccttgataa cccattatca ggcggagagg cttaaggaat tgtaaaaatc |
| | 7081 gtcaatacgg gatcgaattc tgctataata tttacaaaac agaaaggggc ggtacgcaca |
| | 7141 tagacagcag gactaaggca ttagaggcgg cgcaggcagc gctcgataaa aaggcaaaag |
| | 7201 agacggtaat cctcgaactg aaagacctta cgataatcgc ggattatttc gttatctgtt |
| | 7261 ccggcgaaag cactacgcag gtcaaggcaa tcgtcgaaaa catagagaag atattaagag |
| | 7321 agcacaagca aaggccatta gggatcgagg ggctgaggtc cgccggatgg gtcttgatgg |
| | 7381 attacgggga tgtgatagtc cacgtgttcg aagaagatac gagggcgtat tacgagcttg |
| | 7441 agaaattctg gctggacgcc ccgaaaatag cggtagaaga caagtccgct aagaaggccg |
| | 7501 cgggaagcaa aaagcagtat gtatgaaggc gcggatatta tgggtcggaa gaacgaagga |
| | 7561 aaaatatttg acggaaggga ttaaccgtta cctgaaaatg ttaaagcata tggcgaatgt |
| | 7621 ttccgtcata gaggtgaagg aagagaaggg caagacgaag gagaacgccc tttttgcgga |
| | 7681 gggagagcgt atattgcgcc aaacagcgtc ttatatcctg ctcgatgaga ccgggaagga |
| | 7741 attcagttct aaggatttcg cgaagcttct cggcgaaagg gatcaggtcg attttgtgat |
| | 7801 gggaggcgcg tacggggttt cctctgaagt taaggcaaag gcaacgggta aaatagcgct |
| | 7861 ctcgaaaatg acctttaccc atgagatggc aaggctgata tttttggagc agttttaccg |
| | 7921 cgcgatgaca atcgtcaaag gcaaggagta tcatcactga tgggcaagat agcgatactt |
| | 7981 gtatttttgt tgttcctcgg agtcctcggt ttttttgcgg tcgagaacaa agacgccgtg |
| | 8041 atcatgaaaa ttcccttcgg cgacgcttat gagatcccga agatagcgct tgttctcctt |
| | 8101 tcctccacca taggcgcgct tgccgtcctt atcgtgtttt ttataagaga tacgaaacgc |
| | 8161 gtaatcgaaa atctccagta tcagaggagg cagaagaaag aggccaggat tcaggaattt |
| | 8221 tattccaagg cgttgaacgc cataatgggc gataaggagg aggaggcgcg ggaagccctc |
| | 8281 ggcgatatat tgaaggagga cccggaatat atagacgccc ttttacggct cggcgacatc |
| | 8341 tcgctcaata acggaaaata tgaaaccgcg ttcgattatt acaaaaaggt gaaggacata |
| | 8401 aacccgaaga atctacaggt gcttttttcc atggaaacag tgatggaaaa gctgcagaga |
| | 8461 tacaacgacg cgtttaatta cctcgaagag atactcgatc tcgatcccga aaatcttacc |
| | 8521 gggctctaca agaagcgctc tctcctcgaa aggaaagaga tgtgggacga cctcctttcc |
| | 8581 acgcagaagg cgataatcaa gctcgaacat aacgagcacg acaggcagcg ggaagagcgc |
| | 8641 aggatgctcg gttataaata tgaatacgcc cgcgcgagcc ttgaaaacgg agatgttgaa |
| | 8701 aaggcggaaa aggcgttcag gacaatgctc aagatggacg gtaattttgt cccggcgcac |
| | 8761 ctcggtcttg cggaagtcat acttatgaaa ggagagaccg aggaggcgat aaactttctc |
| | 8821 gaaaaggcgt acgagcagct caggtcgata ataataatcg ctcgccttga agaccttctc |
| | 8881 atcaacgtag gagaaccggg caggcttata agattctata aaaacgccat agctaaaaat |
| | 8941 cctcaggaca acgggctgag atttctcctc ggcaagctct actacaggct tgaaatggtc |
| | 9001 gatgacgcca tggagatgct gaattcaatc gataccacgg tcttttcggt gcccgagctg |
| | 9061 tatagtttga agggagagtt atatataaag aggagccaga tacagaaggc gctggacgaa |
| | 9121 cttaaaaagg catgcggcat aaaaaggccg tttaagatcc aatactgctg ctcaaactgc |
| | 9181 ggtttaaaat ccgaagactg gtcgggaagg tgtcccggat gcacggagtg gaatacctac |
| | 9241 aggctcgacg tttatggctc ctgcaaagcg taaagacagc gatgagttat cagtgatgag |
| | 9301 tgatgagtta aaaaacaaaa aaaaatcaaa gacgaaaagt aaaagctcaa aagcaagcac |
| | 9361 atcaccgtcg ttaaaagtcg tactcgatac gagccttttc gtaaatccgg acgtcaggac |
| | 9421 aagcctcgga gcaaccccca cagaggcttt ggaaacattc ctttttctgg cggcgcagat |
| | 9481 acatatactc gaattctaca tgcctccgtc gatattcgaa gaactcctgc attttgtgga |
| | 9541 gagggacaag ataccggccg atctccttct tatcttgcat cagaaaccgc ctaaaaaaca |
| | 9601 tgaactcacg actcccgcct ttctcttata cgaattgatt gaggacataa gggaaagggt |
| | 9661 gaacaagggg ctgcggatag cagaaaaggc cgtaaggagt acagatcaaa aaaagacgga |
| | 9721 tgaaatcgtt caggatatga gaagaaagta cagggacgcg cttagagaag ggatcatcga |
| | 9781 cagcaaggaa gacgtcgatc tcctgctgct cgccatggaa cttgacgccc tgcttatcac |
| | 9841 ggccgatcag gggctgataa agtgggccga aaaattaggt gtaagatggc tgttcccgga |
| | 9901 gaaattcaag gaatatttaa tgagcgcgat aaaaagaacc gaacttcttt ccgcaaaaga |
| | 9961 ataaagtgta gaaacaatgt agttttggga agattctttg atggaaaaag aacttgtttt |
| | 10021 aggcatcgat tatgggggca aatacacagg ccttggggtg gttgaccgca ggaacaatca |
| | 10081 ggtgttgtat gcgaggacca taaaaatgcg ggatgatgtt gcagacatcc ttaaaggacg |
| | 10141 cagggaacag agaggtatca gaagaactca gcagacaagg aagaaaagac tgcgggagct |
| | 10201 taaaaaatat ctaaatagta taggatatga taattctacc gaactattta aatctattta |
| | 10261 tagcctcgcc cataagaggg gctatgacta tgccgatatg ccaacaccgg aagaaataga |
| | 10321 ggaaatggat gagaaggaaa aaaaagaatg gaaagatacg cagagaaatt caaggtacag |
| | 10381 gaaagaggtt ttggcagatg tccgcaaggt aatgtccaat ggtggagctt cggatgaaca |
| | 10441 gataaagagg gtagaaagta ttttcaataa gcagtatcgg cctaagagat ttaataacag |
| | 10501 gattttgacc aagtgcaagg tctgcggaaa aaatacgcca ttaagaagaa atgtgaggga |
| | 10561 gttgcttctt gaaaatatag ttcgcttcct gccgttagaa agcgaactaa aagaaactct |
| | 10621 taagcgaaca attcttgaag gacagcaagg aaatatcaat aaactattca ggaaattgaa |
| | 10681 atttaaccag aaagactggc cgggcaaaaa tcttacggac atagcaaaga acaaactgcc |
| | 10741 gggcagactg cctttttgca aggaacattt tgcagagaat gagaagttta ccactataga |
| | 10801 gaaatcaact ttcaggcttg cgccttcttt aaagacaaag atagaaaatg tcctgactgt |
| | 10861 gattaaagac gaagtaatgc ctaactttgc tctcgatagg gtagtaatgg aatctaacaa |
| | 10921 tttcgatata gctgcaaaaa cgaagggcaa aaagaggttg gcaaaggagg aatacagcaa |
| | 10981 ggggcataag gaaaatagag aaactcttat ggaatccctt ttaagggaga ctgatggaag |
| | 11041 atgtgtttat tgcggtaaga ctataacttt agccgatgca aataaagacc atatatatcc |
| | 11101 taagaaagca ggaggcagca atatctttgc taaccttgtg gcttgctgca ggagttgcaa |
| | 11161 tgaaaataaa ggggggcgga ctccatcgga aagcggcatt atgcctaatc cagaggtcgt |
| | 11221 tgcaacgatt aaaaacgatt taaagaaaaa aatcctcgat gatgcaagaa gcataaagca |
| | 11281 gttagatttc aacaaataca tgtctcatgc ctcgattggc tggaggcata tgagggacag |
| | 11341 gctgaaagag ctaaccggaa acgataaatt gcctgtagag cggctgagcg gtattgttac |
| | 11401 agcatacttt agaaggtggt ggggatttaa gaaggaaagg gctaacgata agcatcacgc |
| | 11461 gcttgatgct gttattcttg cttcaaggaa agattatacg gatgaggggc tggtggctat |
| | 11521 gacgcttaag cctgcaaatt cggatggacg ggagttcgac cctgaaaagc atatcaaaga |
| | 11581 atcggaagag tttaagagaa acaagggcag cagagggtct gccctctacg acaaaaaccc |
| | 11641 gttgtccatt aagaatggca aaattgctcg aagatatatg gttacggaga ttgaaagagg |
| | 11701 gaaggaagat gctgttattt ctgaggaatg gcgtgaaaag ctgaaagaag catttgaccg |
| | 11761 cttcggggtc agcaatggaa aatgtttaac tgatttacag actaaggaag ttgggttata |
| | 11821 tggtcagaaa aatcctatga gccttaaatg cgctgtcagg ggcgcaggca aagggcagat |
| | 11881 tgtgctgata ggcaacaatg catttaagac gaatgttcat aatgtcggcg tggcggttta |
| | 11941 tctcgacgaa aagggcaaga agcgggcatg cgaacttaaa aaccagagac tggcaaaaca |
| | 12001 ctttgtagag ccacaggatg agattaaagg aaagatatta tttaccttga gaaaagggga |
| | 12061 cacagttaag gcggaagatg gaaatattta caggatactt gaacttggcg aaaggcctgt |
| | 12121 tgtagacata aagtgggtac caacatctga tggcaagaaa aagagagtaa agacagctat |
| | 12181 acacgcaaca aagctcacaa agctctaact gaaaattttt ttaatgtcct aacccacaat |
| | 12241 gaaggtgtca ttatcgactg tagttttagt ttggcttagg ctggacaaac ccaagcgtac |
| | 12301 attccttcat catataggcg ttttacaatt aaggctcttt ttgattttcg caaaccccct |
| | 12361 ggggtttatt tcaaaagatg gcttttagaa aattattgaa aattttctct gaaaacttct |
| | 12421 tgaaaatact aagctataag gacggtttca atcatcattt tcaaagagcc ttaactgtaa |
| | 12481 tgtgctgccg tcctgtgaga gggctatctg cggtagtttc aatcatcatt ttcaaagagc |
| | 12541 cttaactgta acgctcatat aagcaaggtc tgtccgccgg agaggacggt gttttctcca |
| | 12601 tcggtcgcgc tactgaacca actgactgtc agttagacga aacaatcaga acagagtccc |
| | 12661 tgccggaaac cgtttccggt tgtccagcga acattgagaa agctgcgacg ccagatgcac |
| | 12721 cttccggtgg gaagtcctcc gctcaaacgc ctgaagcgtt tcgcagcctt tctgaaacaa |
| | 12781 atcaaaaaag ctgagacaag attcctttaa aatagagcat ctcaaagaaa agtccctgcg |
| | 12841 agcaatgcgc ttcttgaagg aagcccctct ctaacgtccg aaacgctcag ggacactaaa |
| | 12901 atatatctca gaattgtaaa agagcctaaa ctataatatg attacattat atgcctttaa |
| | 12961 atttcaagag ctttagcgga ggtggatatg caaagcaata cttttaaaat ttcacgcatc |
| | 13021 catcctgaat acgtgcctaa agaaaaattg cctcttgccc aattcaatct ttttacaaac |
| | 13081 gaagcggaga gcattccctt attgatcgac aaagatgttg acgacgagat atttatcgat |
| | 13141 gaagggattt ctctgataaa aagcggcgat tcatcgcaga tagtgttatc ggggttcggc |
| | 13201 atttttttaa gcaaaaagag cgagcgtctt attgtaaaga aaaacactaa agttgtttat |
| | 13261 gagtttccct ttttcaggct gaacgaagtc atcatggctt cccgcggcat atcgctgtcg |
| | 13321 tcggatctta tagaggaatt gtgcgagagg ggtataagac tgaattttct gaacaacaac |
| | 13381 ggcaagccgt acgcaatgct ttcttctccg atgctctccg caaccgtcat atcaagaaga |
| | 13441 gagcagatta ttgcctttac cgatacgcgc ggtcttcaat tctcaaaagc tattgtggag |
| | 13501 ggcaaaatca gaaatcaaga aagacttttg agatatttcg gcaaatatat taaaactgtt |
| | 13561 gacagcgcca ggtttgacag aatagaggag ataacagatg tcattaggaa gatgagcagg |
| | 13621 caggtggaag gtattcacgg gcagagcatt gaagatgcaa gaggcagact gatggcaatt |
| | 13681 gaaggcgctt caggtaggct atactgggac ggcgttaagg agataatcgg aaatagaata |
| | 13741 gagtttttcg ggcgggaaac gcggggagcc attgatgccg taaattccct gctgaattac |
| | 13801 ggctatggga ttttatattc ccacgtctgg ggcgccgtta taaacgccgg gcttgagccg |
| | 13861 tttgcggggt ttcttcatgt ggacaggccc ggaaagccgt ctcttgtttt ggatttggtc |
| | 13921 gaagagtttc gtcaacctat tatcgatagg attgtaatcg cccatattaa tcttggagaa |
| | 13981 tcaatcgaaa ttagaaatgg cctgcttgaa gctgagacca gaaagctgat agggaacaaa |
| | 14041 attattgaaa ggctggagag tcaggaaaca cttgacggca aaaaatataa aattaggtcg |
| | 14101 ataatccaga tgcaggccag aaatttagcg gcctttctga acggcaaaag agaatacaaa |
| | 14161 acgttcagat ttaagtggta attgatggaa gaacatacag tctatatttt ttacgatata |
| | 14221 gaagatgacg gcatacgcaa taagattgcg gagaagtgta aagactatgg gctcgaaaga |
| | 14281 atacagttca gcggcttttc cggcatttta aatagaaaca agcgggaaga actatttcta |
| | 14341 aggctcattt ctttaattgg cggcaaatca ggcaaaattc ttatgcttcc cttatgtgaa |
| | 14401 agggatacga aggtaagaag ggagtttata caggaaggac aagatgattc aactgagggt |
| | 14461 cggtgatctc aagcagttcg tatattgtcc aagagtcgtg ttctacaatt atgtgatgcc |
| | 14521 tgtcgagaaa gttccaacct tcaagatgga gcatgggaag atagcagagg ataaaataga |
| | 14581 caggcttgag cacagaagaa aacttaaaaa gtacggcttg tccggcgggg aaagggaatt |
| | 14641 ccacttgaat ttatattctc cgaaatatga gttatcaggc aagattgatc tgcttatcaa |
| | 14701 tactgtcggt tcttattatc cggttgactt taaatatacc acctcgcagc cgcataaaaa |
| | 14761 tcatatctat caacttttgg ggtatgccct aatacttgaa gacatatata aatgcaaagt |
| | 14821 caatagggga tttgtatata taattcccaa agaggatgca atttcttttg acttaacgga |
| | 14881 tgatttgaag ggcaaagcag aaattttgct tgatgagatc agagatatga tagcattgca |
| | 14941 gcaaatgcct gcccctgcaa actccagaaa taaatgtgac gactgcgaat ataggaattt |
| | 15001 ctgcggagat attttttgac aattataaac cgccgagggc tttatacagc cctatcacga |
| | 15061 attccgtcat cctctcgtaa tcgagcgttt cgaatgtatc tcccggcgca tggtaatgag |
| | 15121 gattcctgta aaatgccgtg tccgtgatca tgaacgccga atagccgaac tcccagaagc |
| | 15181 cccagtggtc cgagaaatta acgccgggga tcagcgaaat cccgttgaac gattcagcgg |
| | 15241 gcagtgaaga tgactcttga aaatatctct tcactttttt cgtaaaagac atggagcgga |
| | 15301 tatttccgac aaacgtaata aaattgccct tgtcaggata aaaccatttg aatatcggaa |
| | 15361 aaggataata ctgagagttt tttctgtcgg agtaatatcc gagcatttcg agggagatca |
| | 15421 tgccgcagac ttttatgcct tcctgtttaa ggcttttagc ataaacgtag cttcccatat |
| | 15481 gttttgtcat aaaagtggga ggctcttcaa gactgaaggc aacaaagcgg accgtccttt |
| | 15541 ggaaagattt tgttacggca agccgcgcca gttcgagcag tcccgcaatg ccgcttgcat |
| | 15601 tatcgtccgc gcccggagtg cccgttacgg tgtcgtaatg cgcgcctatt acgagaatcc |
| | 15661 cgtcttgcgg gtttccggcg ccttttattt cgcaggatac gttgtaatat gtattgccga |
| | 15721 gatacccaaa cggctgcttt ctaacctcac agccgtaaga gcgaaatttt ttttctatgt |
| | 15781 aatcggcggc cttgttgagc ttatctgtat cgcgataact cctctcgcct atatcgtggg |
| | 15841 cgaggcgctt gacggtttcg ataagattag cttttatatt ttccattttg ccgattaatg |
| | 15901 ttcatagctt tataaattct ttcggcgcta aattcgtcct cattgtccat accgatgcag |
| | 15961 tgccgttttt caagcgatac acaaccagcg ggtcatagcc gtattgctcc acaaagggtt |
| | 16021 tcagaaattc gcgcttttgc actatctcct tctttagttc aatatcttcc gtaagttcaa |
| | 16081 cgaccccgct gactcttatt tgtataaggt tctcaaagct gccgttgtta aagctcagct |
| | 16141 cgaccagcgg gttttcggtc aattgtttat gcaagtcttt cacctttccc gtgtgaaaca |
| | 16201 aaataccgtt ctcatcagcc ctatacatca gcataccgcg aacgcggggc ttatcacctt |
| | 16261 caatcgttgc catgtgaaat accgggtttg cattaagaaa ctccaaaatt tcagtcttgt |
| | 16321 tcatataagc ttcctcctaa cgataaatgt tttcaagcct gcggataata tcgagttttt |
| | 16381 ctttctctgt tgttctcact cagggttact ccatttgcgg ccctttgcct atcttcattt |
| | 16441 tgcccctgaa atatctttgt ttgacttccg cggtcttttc cttccatttg tccacgccgt |
| | 16501 gctgtttaat gtagcacaaa gcaaatagtt taatgttgtg cggcaggtcg ccggccttgt |
| | 16561 ccgttgcggg atttaatttg gcgcagggga attccgcgca ctcgaagcag aagtcatgtc |
| | 16621 cgcgtttgct gatgcaggca taggtctcgc atgtgccctc aacaaactgg cagcgtccct |
| | 16681 tgagcggcct gcatccctga catgggatgc cgttccagtt aaattttttg gtcagcattt |
| | 16741 ctctccacga aggatcgtcc tttactttgt acgctgggca gtcaccgcag tatcccccgc |
| | 16801 acggcgcaac taatgtgatg tcatcttttg tttccattcg ataagcctcc tgtagccggt |
| | 16861 ctgtatacct ttgcctggtt gtcgcgatat agttgatcga aaagttcagt aaattgggat |
| | 16921 tgtccggcca tcatatcctc ttcttagatt acacagcact aagctgtttt ttcaattcag |
| | 16981 gagtgtgagt taacatcggg acagcgacca ggaaaggccg ctgtcccgtt gcggtcatac |
| | 17041 taacaacagc ccttgtctga ttttgtcccg cagcaggact ggaacatgtt cttcatattt |
| | 17101 ccatccgtgc agcacttctc aaaaatggtt ttaatttttt cttttacgtc ttgaccggtg |
| | 17161 atctcaagac gatacccatt ctcgagttcc gtaacgttag cgctacagca ttgtttctcc |
| | 17221 attattattc acctccttcc agtcttttat tattcgagaa cactcgaaca agaccgccaa |
| | 17281 aaaaaactat tccatgaact tttttatgga atcgatcacg tccttattta ctctgcaacg |
| | 17341 atagatctgc ccctcgcgct cacatgttat aagcccggca ttttggagtt ccttgaaatg |
| | 17401 atgcgatatt gttgaccgtg aaagattcat gcaatcacag atctttgtaa aggccttttc |
| | 17461 catgcttcca tcagacgcgc aacatgttgc ccctttaatg cgaaattccg ccccggcctt |
| | 17521 ggacccctcg attccgcaac agtttttgta gagcataatg aatatcttca atcgctgctc |
| | 17581 atttgaaagg gctttaaata tttttgtgta gttagatagt ttcacaacat tagaataaac |
| | 17641 ttatcgtgcg tttttgtcaa gcattttgac gagaggtaaa agctttggcg ttattatcaa |
| | 17701 gagtatgaag gagtaggggg ataaaatcgg ctgaaatttt tacgaatgat ataccgaagc |
| | 17761 cttatgaaat tcttcaatcg ccatatcctt atcgtatgag aagccggcag gaaaaatgcc |
| | 17821 cttctttttg caatacacaa ccatctcatc cagcacagcc atgagttcat ctttcaggtt |
| | 17881 gttcacttcc cctaagccgt cacaacccct ctgcctttct tttataaaac tatatatctg |
| | 17941 aacgtagtcc ttagcttgtg cgtagagtcc tgaaagatga gaatcggatt ccgccatttt |
| | 18001 agtaattgca tcaatcatgc atacacctcc tttcgactgt atagacgaag gaaagtggga |
| | 18061 aaaggataca cccttatttt ttcttgtgga attttaaggt gggttctttg cggtcgcagg |
| | 18121 cgaattcgtt ccgctcgtcg cggtagaaac tgcagtgcgc ttccagcgtt tttttcagcc |
| | 18181 ttgcccgcgc acggtggagc ctgatcttga cggtatcaag gcttaccccg agaatctctg |
| | 18241 ctatttctcc gtttttgaac ccttcaagct cgctcaggac aagtatggtc cgggaatgag |
| | 18301 ggggaagggc atctacatac ccccggatac attcgctcat ttccttgcgg atcaattgca |
| | 18361 catcgggggg aggagttttt ttatcataaa aaacatctgt ttcttcatta tccataccga |
| | 18421 gcggctgttc ttgtctgaac gacggactgc gcatcctgtc gactgcagcg ttcgttgcaa |
| | 18481 ttcggtatac ccatgtcgac agccgggaat tttttctgaa atccctgagt ccacgatgaa |
| | 18541 cctttacgaa aacttcctgg gctgcatcct cggcctcgcc ggccccgatc aaccgctgca |
| | 18601 ggtaacggag aacaggcgtc ctgaattcat tataaatttg ttcaaatggc gtatcgttgg |
| | 18661 tcatcgattg ttgatctgct ttttaatctc ctgcatagga gtcattcctt ccccaaaatt |
| | 18721 aaatcaacca tgattttacg aggttggcag aaaagttttt ttactctttc cctatattct |
| | 18781 tcgggtgtaa cgtgttcctg atgagttcct atcacgattt taatattcgg gaatgcctct |
| | 18841 tccaaagccg atttgtatgt ctctttaaac gggcatagcg ccttcatgca gtaagtaaag |
| | 18901 tgtatcgcat ccacccggaa ttcagtcagt gcccttatac gttgcaataa tttatcggcg |
| | 18961 ccgctaaggg tagggcagcc cggacaatta ataatcccta ccaaatctaa cgggtcatct |
| | 19021 tttggatagt ccgcaaatgt tcctcttctt tttctgagat cggcaagaca gcttgccgac |
| | 19081 gaacagccca agtcctgcgt cgcattgctg catgtcaata ttccgattcg cgccatataa |
| | 19141 tcaccttctc gtttacagtt tctctaaaaa ctctttcctc acaggcgacc acgcgtcaac |
| | 19201 ggccttgcaa ggcatgttgc ctgtaaaggc cgaatgaacg gcgtttgacg gtatcgcgat |
| | 19261 tacatcccct gccttcaagg tagttgtttc tccttcatag gtaaaggtca attccccttc |
| | 19321 cagtatcagg gtgatctgtt ccgcctcgtg cgaatgctca ggaaattttg tgtccggctc |
| | 19381 catctcaaaa tatgtaagca tcgccttatc gagagctatt gcccacatcg aggctcccgg |
| | 19441 gacattgctt ctgagcttta attctccttt gcgataaata tgtatctcgc ccattgcttc |
| | 19501 tccccttttt agcgatttca tttttttaga tatttcatct gaatattcca gagtataagc |
| | 19561 cccgcgcctt cgggtctcga aggcagaaat tttctaattt cagaggcgat aaattcggca |
| | 19621 ttgtcggatt tggccgcaag ctctttggcc tttttatcaa aggcaataag atagtctttc |
| | 19681 atgtcttcta tatctttttt tgttgagacc ggcccgtgcc ccggaattat tttctcgacg |
| | 19741 tccatcgtca tgatatagtc gagcgttttt aaccatccct ctatatcgcc ttctcccatg |
| | 19801 aacgggtgat aaccggtaaa tagaatatcc cccgcgaata gaattttttt gtccggcaga |
| | 19861 tagaccataa tgcttccatc ggtatgcgac tgcctgaaat agatgagttc tattttccgg |
| | 19921 tcgccgaggt ctatttccat tctttcgtta aacgtcaaaa cagggtatgc gatcttcgtc |
| | 19981 cctttcacat ccttttcggt aagtccgtag gtatttgagt ttttcagggc tgtttcgccg |
| | 20041 cgaattttca gattattacc gtcattagtg tgcgagatga tcagcgctcc gagttttgcg |
| | 20101 aattccgagt tgccgaacgt gtggtcgagg tgataatggg tgttgaggac atatttaacg |
| | 20161 ggtttcttcg agactgacct tatgtccctg ataaaccttt tcgcctcttt tgcggatatg |
| | 20221 agggtatcta ctaccacaat gccgtcttta ccgataatga tgcccgcatt agcgccgtag |
| | 20281 ctgttctgcg gagtgctgtt tttagtatcc acatacgaat aaacgtcgtc ggctatcttt |
| | 20341 gtcagtttct ccgcggcaat tgcaatttga ccaaatgcta aagaaacgat gaatacaaat |
| | 20401 acccaaatcc tgaaaatcat caaccttttc ataacatcct cctcgttttt tattttaatc |
| | 20461 ctttaccgaa tccttcaaca gtttcctgaa aacaaccata ttcgattttg tgaagccgtg |
| | 20521 cttccgataa aattcatgcg cctgctcgtt atcattatcg gtaagcaggg tgatccttcc |
| | 20581 gcatccgtta ttaatcgcaa attctaccgc atgcttaatt aaacttgtac ccacgccttt |
| | 20641 cccccttgaa tcgggcgata caaccatatc ttcaagaagg gcgactttct tgccgagggc |
| | 20701 ggtacttatc gtgaataata cgactaccat tccctgaatg acgccgttag attcgcatac |
| | 20761 aaatacagag ccggtttcag ggttcttaac gatcatctcc aatcccctcc tttgagccgt |
| | 20821 cggattgggt ttaaactcat gctcttgacc gaaaagaagg ccgagaagct caatgcaaga |
| | 20881 gccgagatcg tctgtatttg caggtctaac ggttttcatt aatatgcttt ctctcgtccg |
| | 20941 atcacttcat tctcaagaat tcttcaatca ccggacacaa ttcgctccct ttttcttcct |
| | 21001 gcacaaaatg tcctacatca tcatatttta ttgttttgga atcggagaaa aggcttatcc |
| | 21061 atctgctcag ttcctgctct ctgaaagcaa tgtctttcat tccccataaa atgagagccg |
| | 21121 gcttatcttt tattttatca ctctgcgacc agagaaaatc cagccaatca ctggaatcaa |
| | 21181 taatccgctt cggaaatgtc cagcaaccct tccgttgctc cggtatttca agcgccttga |
| | 21241 aataatgctg atggattgaa gtggggagtt tggaggtatc acccatcgcc tttttcatta |
| | 21301 tgacccgcac aaaaaaatta aagcgtttga tcaagaacct gccgattggt ccgcccacaa |
| | 21361 atttgctgaa cctctcataa taaggatctc ccttgacaga ccacatccag gtattcatga |
| | 21421 tgatgatacg tttcatattt tccggagagt tgacggcata tgacaagccg ataggtccgc |
| | 21481 cccagtcctg aacaacgagc gttattcctt tcaaatctaa tttttctatc aatagtttca |
| | 21541 gatttttcgc atgatcttcc ggataatacg accaattcca cggcttatcc gataaaccga |
| | 21601 acccgataag gtcaacagct atgcagcgat aatttttcga gagacattta atcagatgcc |
| | 21661 tgtatagaaa agaccatgcg ggattgccgt gaaccataac tatcggctct ccgctgccct |
| | 21721 catcaacata atgcattttc cccatttcaa gttcaagaaa atgagactta aacggatact |
| | 21781 cttttctgtc tatccactta ttcatgaaac ctccttatct ctcatctcct ttttcgctcc |
| | 21841 gtgttcatag cctgaccgat tcgcgccgtt ataaaccggt atttgccaag tcggtccatg |
| | 21901 gagagagaac taccgccaaa catttcgatt atatttccac aacccattct tttatccagt |
| | 21961 tgctctcaag taaatcctga ataacaaaag ggtcgtttat tataatccta tttgcatcgt |
| | 22021 caacgctttc ggcctcaaat gtaatcattc cacccgtcct gtctgcaaaa ggccctccga |
| | 22081 gatacttgct caatttaagc ttgtgccaat actcaatatg caacggtaca gcatccttta |
| | 22141 tcttttccgg ctcctttttc atgaaataaa aaaatgcaaa ttgttttccc atgccatcct |
| | 22201 ccctgcag (SEQ ID NO: 43) |
| MGQA01000 | ATTGGGTGGGATAGCATGGATTTGTAATCCTTTTCCCTCAGCTCAGCGAATTTTTTATATCTTGGATCCTTTGA |
| 002.1_ | TACATCTTTTACATAGATTGGCTTTCCTTCTTTTGCCGCTGATCCTGTAACTCCTTCTCCCAATTTGAGCCTTAC |
| organized | CTTGCCAATCGCCTCTTTTACAAAGCTTGTGGTTGCCGTTAAGACAAGTTCTTTTCCATTCCAGAGGTATATTG |
| | AGGAAACATCTACGCCCAATCTCTTTGCGGTCTTTTCTACGACTGACTGGAGCACCTCATCAAGCGCATAATC |
| | AGAGCTTGCAAGCATGCTTATCTCTTCAAGGGTCATAATCTCCATTTTTTCCCTGTTCGCCCTTTCCATCTGAA |
| | AGACAATGCCGCCTTCTTCACTAATCTTTACATGAGAAAAAGCATCGCTTGGATAATATGGATTGATAGCCTT |
| | GCTTGCTGACGGCATTGTCTTATATTCCCAAAAACCGCATGCGCACACAAGCTTTACCCTGTAATCGTCAACC |
| | CTCTCGCTTTTCATCTTCTTTCCGCATTTCCTGCATCGTGTAATAAATTCCATTCAGCGCCTCGTTAAAATCAGT |
| | TGGAATTTAACAGGCTGTTGAAAAAGCCATCAACAGCCTTGATTACACAGATTAGAGAAAAAGATTACACAG |
| | ATATTTACAGAAATTTCAAGGAGTTTTAATCTGTGTAATCTTGTCTTTTATCTGTGTAATCCCGAATAGTCGGG |
| | ATGTAATCGGAGATTGTTGAGTTTTTCAACAAGCTCTTAATATTCGGGAGTCGGTTCTTCAGTCTATAACCTGA |
| | GGTATTTCTTTGTTCTCTTCCGAAATATTAGGCAAGACGAGTTTTACGACCTTGCCTTCGTCACCAAGTCTGCC |
| | AAGTGGATTGCCATTCAGCGTCACATTGACACCGCCTGCATTCCCTATCAGTATAGAAAATTTTTCTTTTGCTA |
| | TCCATTTAACCTTTTCGCCTTCTCTGAGAGATACCTCAAACGGATCCTGACTATCCACTTCAGCTCGAATCCAT |
| | GTGCTTTTCGTGGCTTGTATAATCAAGGCCATGGGTTCTTTTAAGTCTGCCTTAGATGACACAGATTCTTTTGT |
| | ATTTTTATCCCCTTTTTGTAAAGCCGAAGGGCTAACAAGTATGCCTCTATCGTCTTCCTGTTTAGACCGGGCCT |
| | CGTAAGCTATAGCTTCCTTCTTGTTGCTATCAGATACATCGTTGCCAGTCGTTAGCTGTATTTGTGAATAATCA |
| | GGAGCATCTGGTTTATACTTTTTTAAGATTACATATGCTCCAGCTGAAATTATTAAAAGGATAGTGACAGCTA |
| | TAGCAATAAGCGATTGCGTAGATAATGTGGTTAGTCTCTCTAACTTTTCGTCTTTTGCTTTGGCAGCCTCAACA |
| | GGTTCGACCTTACTTTCAGTAAGGGTTTTCATATACACTTCATAACGAAGCAGCGCATCATGTCCGTCTACAC |
| | CAAGATGTTTGCAATAAGCCTGTATAAAACCTTTTACAAAAGGCGGTGCGGGTAATTTGCTAAAATCGTCTTT |
| | TTCTAATGCATTAAGCAGCCCAGCCCTTATTTTAGTTATTTTAGCCACATCTTGTAATGAGATACCTCTAATTT |
| | CTCTTTCTCTTTTAAGATATTCGCCTATGCTCTCCATTTTGCTAACTCCTAATTATTTATGTTGAAGGCTTTTGC |
| | CTATTTAATGTTTATTATTTCAAGAGGTCAATATATCCCTTAGCCGACCTTGCCATCTCGCTGTCGGGGACAAG |
| | TTTAATAACTTCTTGGAATGCATTCGCCGCGCCTTTCTTGTCTTTAATCTTTATGAGAACAAGACCGAGGTTGT |
| | AATGCGCATCCGCATAATTTGGCGCGTTCTTAATTACGAGTCTGAAAGTATCAGCTGCCTCTTTATCTTTATTC |
| | ATTCTCATATAGGTAAGGCCAAGGCTATTATATGCCTGCAAATACATTGAATTGTTTTTAATTGCCTTTTTAAA |
| | ACTCTCTTCTGCCTCTACATAATCTGTTTTTTCGTAAAATGCTCGTCCCATATTAAAATAGGCAAGCTCTGGAG |
| | TTGTATAAAGGATGTTTGCGAGCGCTTTTTGGGCCGCTGCGATAGCAGCATCCCACTCTTTTTTTTCAATGTAG |
| | ACAGCTGAGAGGTTTGTGCTGGCATCAGAAAATTTTGGGTCTATTCGGATGGCCTTTTTAAAGTGCTTTATCG |
| | CATCATCATGGAGGCCTTTCGCAAAATAAGCAAGCCCCAGGGCATTATGATACGATGCCTCATCAGGATATTT |
| | TTCAACAGCAGTTATCAGTTCTTTCAGGGCTTCAGGGATATTTCTTTCATTGAGATGAACAACCCCAAGCTTAT |
| | AATGTATATCTGCATCTTCCTTTCTATTTGCCTTTGAGCCTGCACATCCGTAAAAGACAGTTGCCAGGAGGCA |
| | GTAAGCGGTAAGCAGTAAGCAGTGGGTAATAAACAGTAACTGCCTACTGTATACTGCGTACTGCATACTTTCT |
| | TTGACTGCTGGTTTCTGACTTCCAGTTTCTAAATTCTTAATCATCTCATACTCCTTAGCCTCTTGTCGGTTTTCA |
| | AGATATATCTTCAAAATGGGTTAATTGCTTAAATTTTCTATATCTATCGTAAATCTCTTTTAGTGTCAGGGTGC |
| | GCATTCTGTTGAGGCTGAAATCCTCTACATTAAATGAGGCCATGACACTCCCAAAGATAATTGCCTGTCTTAT |
| | ATTAGATTCATTTGTGTTGTCAATATTTGCAAGATATCCTATCAGTCCTCCAGCAAAACTGTCGCCAGCGCCG |
| | GTTGGGTCAAATACAGATTCCAGGGGATAGGCAGGTGCTGAAAATATAGAACCACCATTGAACATCAATGCG |
| | CCGTATTCCCCTCTTTTTATTATTACTGTTTTAGGGCCATATGCAAGAATTTTTTTTGCTGCCTTTACAAGGTTG |
| | GGTTCTTTTGCAAACTCTCGCGCCTCACCTTCATTCAGGACAAACAGGTCGACTTTCGACAAGAGCCCCTTAA |
| | GCGCATCAGGCTTGCCTTCTATCCAGAAATTCATTGTATCACATGCAACGAATTTTGGCTTTTTGACTTGTTCG |
| | AGAACATTCAATTGAATCTCTGGGTCAATATTTGCAAGAAACACATAGGGTGATTTTGTATAGACCGCTGGAA |
| | TATGAGGTTTAAATTTTTTAAAAACATTTAGGTGAGTTGCAATGGTATGGGCTTCGTTTAAATCATACTCATAT |
| | CGCCCCTGCCAGCGAAATGTCTTGCCAGCTATTTTTTGTAAGCCGTTTGTATCTACCCCTTTTTCTTTTAAAAA |
| | ATTAATATGCTCATCTGGAAAATCTTCACCTATAACAGCTACGAGGCTGACATCTGTAAAATAACTGGCAGCC |
| | GTAGAAAAATAAGTGGCTGAACCGCCAAGGACATTGTCTGCCTTTCCAAAAGGTGTTTCAACTGAATCAAGT |
| | GCAACAGAACCTACAACCAGGATGCCCATCAAAATCCTCCTGAATTTCAAGTAGGCAGTATGCAGTAAGGAG |
| | TAAGCAGAAAGAAACCTGCCTACTGCCTACCGCTTACTGCTTACTGTTTTTTACCATTATTCATCCACACTGTC |
| | AATTAATGTGGAAAGGGCAACTATCTCAACTTCCCTCTTGAGCATTGGTACCATCTCTTTTATAGCTAAAATG |
| | GTGGTATGGTGTGGATGGCCTATAGCAATGGCAGAGCCTCTCTTTTTTGCAACTTCTATCAATTCTCTTATCTG |
| | TCCCTTTATGTAATCTATATCCTCTTTGTTATCTAAAAATACCTGCCTGCTCAATGTTTTAAGTCCGAGCTCTTT |
| | TGCTAATCTATAGGCAGTTGATTTATTCGTGGTTTTGCTATCCAGAAAGAATAGATTTTTTTCTTTTGCGATTT |
| | CCAATACTATCTTCATAAGTCTTTCATTCTCTGTAAATTTCGAGCCCATATGATTGTTTATTCCAATAATATGC |
| | GGGATTGCGTCAATATCCTTTTTAACCTGTGAAGCAACCTGTTTTTCAGACATGGTTGTAAGTATTGCCCCTTC |
| | CCCTGGATCGTTGTTGCTTAAATCTTTTGGCTCCATGGGGAGATGCAGCAACACCTCTCGCCCGTTTAAATAA |
| | GCCTCCTTTGCAGCGTTCTCTGAATTCGGCAAAAATGGCAATACAGCGATAGCAATGGGGGCATCTATTTCTA |
| | ATATCTCTTTAAGCTGTGCCCTGTCGCGGCCAAGATCGTCTATAATTATTGCTACCTTAGCCAGTGGTATATGC |
| | GGCTTTTGGGGAACAGGAGGTTGAGGTTTCTCTATCTTTGCTACAACTGTAGATGTTGAAATAGGTTCTGGAG |
| | GTTTTTCTTTTGTACTGTAGCTGAGGTATAGAATAAACCCTAATATAGCAATGATAAAAAATACCGTTGCAAT |
| | TCCCAAAACAAAAAGGGTCTTTGATGAGAAAGACCCTTTTTTGCTCCAGTTATTTTTAGCCATTTTTTAACCTA |
| | TATAGCTAATCCCTCTTAGATGAGCCACTCTTATGATTCCTTCTTTATTGTCTCTTTAAATATATACCAACTCTT |
| | CAGATAATCCAATGCCCTTTTGAGCTGCATGTCCGCTTCTGTCTCTTCCTTTTCTTTTACTACCTTTTCCTCTAT |
| | CTTTATCTTTTTTTCTTTAGGAGATTCAACCTCTTCGGCTTCAAGGTGTCTTTCTAGTTCCTTTTCTTTAATATGT |
| | TCTTTTATTGTTTCTCCAACCACAATATCGGGTTCTATGCCTTTTGCCTGTATAGATCTTCCTAAAGGCGTATA |
| | ATATTTAGAGGTTGTAAGTCTAACCGCTGAACCATCGCCTAAAGGAATGATAGTTTGAACAGAACCCTTGCCA |
| | AATGTCTGGGTTCCAAGCACAACCGCCCTTTTATGGTCCTGGAGCGCGCCTGCAACAATTTCTGATGCGCTTG |
| | CGCTTCCACCATTTACCAGGACTATAATCGTATATTGGGGGTGCGTTCCATCTGCCCGTGCCTCAAACTTCATA |
| | TCCTGGCCTGGGCTTCTGCCCTTGGTATATACGATTAAACCTGATTCCAAAAACGTGTCAGCTACTCCTACAG |
| | CCTGCTGCAAAAGTCCACCAGGATTATTCCTTAAGTCAAGGATGAGACCTTTTAACTTGCTGTCCCTTGAGCC |
| | AAGCTTATTTAGCGCCTCTTCAAGTTCATTAGTAGTCTTTTCCTGGAAATGTGCGATTCGGACATAACCAAAC |
| | CCTTCTTCTAACGTTTTATATTTTACACTCTTAATAGCTATGATATCCCTTACAAGTGTAAATTCCTGTGGTTCT |
| | TTAAAGTCCTCCCGCATTATCCAGATAGTAACCTTTGTTCCTTTTTGGCCTCGCATTAGCTTTACAGCATCGTT |
| | TATGGTCATATCCTTTGTCGGCTTATCACCTATTTTTACTATTTTATCTCCAGCCTTTATCCCAGCCCTGAATGC |
| | TGGCGTATCTTCTATGGGCGCAATGACAGTAATTATGCCATCCCTTATTCCCATCTCTATGCCAATACCACCAA |
| | AGGCGCCTTTGGTTTCAACCTGCATTTCTTTATACTCATCTGGAGCCATAAAAGACGAATGCGGGTCGAGCCC |
| | GAGAAGCATGCCTTTTATTGCGCCATAAATGAGGTTTTTGGGGTCAACATCTTCTGCATAGTTATCCTCTACG |
| | ATAGAGATCACGTCGGTAAATATTTTTAGATTCTCATATATCTTTGGAGGCACAGCGGAAACCCTGTGATTAA |
| | TCCCCCATAGAAGAACGCTCGTTATTAGAGCTATTCCCAACAGCGTCATGAGCTTACGCCCTTTTAACCTCTT |
| | GAACATAAATGTATTCCTCCTGAGTTTATTTAAGATAAAGACTTGATGCGATATGCGAAAGGGTTAAAGGAG |
| | GTAGCGCTACCTAATTGCCTTCTGCCTTAAGCCTGACGCTCTCTGTCTTATCATATGTCAACCAATTTACCGGG |
| | TCTAATGGTACACCCTTCTGTCGTATCTCAAAATACAGATGAGGTCCTTTTATTGAACCTGTATCTCCTACAAG |
| | TGCGATTATATCGTCTCTTTCCACGGTAGAATTAACCTCTGCGAGGATTTTTGACAAACGGCCAAAGAGTGTA |
| | TGATACCCATTGCCGTGATCTATAATCAAAACCTTTCCATAGCCCCTGAACCATCCAGAATATACAACGTGGC |
| | CTTTATATACGGACTTAACCTCAGCCCCCATCGCAGCCTCAACCTCGATGCCGTTATTAAAGGTAACAGTGTG |
| | AAATTTGGGATGCTCTACCTTTCCATACATTGAAATGACTTTGCCAGCTACAGGCATTGGCAAATGACCGCGC |
| | ATAGACGCAAAACCGTTCGTCTCATTATCCAAAGGATTATTAACCGTCTCATTCTTCAACCTATCTATGAATA |
| | GTTGAAGCTCTCTGCCTGCGCTTTCCATTTCCTCAATTGCGGCAATCTGTATTTCCTTTTTCTTTTTTATATCAT |
| | TTAAAAGGACCTTTTTCTTGTCTTTTTCTTCTTTTATTTCTTCCTTTTTCTTTTCAACTGCATCTTTTAAAGAGCC |
| | TATCTCCTCCTGAAACTCTTTGAGCCGTATTTTCTCTGCTTCTAAGAGCAGTTGATTCTCATTGTAATCTTTTAA |
| | CAGGGTTGTATCATGATCTATAACCTTGTTTACATATTTATATCTTCTACTAAACTCATTGGCAAAATTTGGGG |
| | CAAAAATGCTCTGGGTTATGCCCGCCCTATTCATCTTGTACATTGCAACAAGCCTTTGTTTTAGAAGTTCCATC |
| | AACCTTATTCTCTCCTGAATCAACTCTTTTATTCGGATATCCACATCAGAGTTTTTTTGCTTTATGACAACAAG |
| | GCTGTTTTCTATCTTTTCTAATTCCTCCTCCTTTTTAGATAAGGCCTTGTCAAGCCTTTCAAGTCCTTCAAGAAT |
| | AGCATATTCTTTTTTTACATTATACTCTAAACTTCTCTTTTTTTCTACAATCTCCCTTTTGAGGGATTCCAGTTG |
| | TTTTTCCTTTTTTACAATCTCCTTCTTTAGCTTGGCAGGTTCAGCCGCATAACCATGAATGGCCGGGGAAACGA |
| | GTAAACACAAAACAAGATAGAAGGGGTAGGTTCTAAGGGTCAATAACAATAATAGGTTTAAATTATGGATTG |
| | CCACAGTTTTAACTTTCTCTTTTTAAGCAGTCATTGGTTTTTTATAGAATTTAATTTATCCTATCTAAAACAATT |
| | AAATTTATACTCTTTATTCTATCTTTCAGCGCCCCCATTTTTGAATCTTTGCCAAGCTCGAGGTTTGTAACAAC |
| | AGCTTTATTTACAGTTCTGAAACCATCTTTATCATAATAGCCAAAAAGCTGATGGAGCGCATAGTAGTTTCCA |
| | TCTACGTTGCCGAGATAGAGCATGATATGACCGTTGAGGGCTAATAAGGTAATGCCTGGGATTGCGGAACTT |
| | ATTGCGTTATCAATATCTCCGTTTGCCGGCATTTCATCAAAACCTGCTAACAGCGTCCCGACATTAGATTGTTT |
| | GCCAGAATGCCTGGGAAGGTTTATGCCCATCGTCGCAAACACATCCTTGATAAAGCTTGAACAGTCCCTCAA |
| | ACCGTTTCTACCTCCCCAGCTGTATTTCTCGCCAAGGATTTTAAATGCCTGTTTTATCACATTTTTCTTGGTATA |
| | GGGAAGATAGCCAATATTCACATCGCTGCCTTTCTTAATAAATGCATCTGCCCATTGAAGCTGACCATTGCCC |
| | ATTTTTTTTGGAAATTTAATAATCCAATGTCTTTTATCTTCACCGCTAAGCGCAAAGGAACGACCCATGGGAA |
| | TGGACTCAAGAACCTTTGCACCCTTATTGTCTGCAAAAACTGCCACTCGGTTTCCTGTTATTAAAACGAATTTT |
| | TCAGCATCTATTATTAAATCGTCTTTGCTATTTACAAAAGCAATGATATTGAGTTTTATCCAGCCACGTACAAT |
| | GTTTGTCTGAAAGAAACCCCACTGTCCATCCCTACTCTTGTGCAGGAGCGCCACCACCTGAGGAGGATATATG |
| | GCGGAATACTGAAATGCATCAAAATCTTTGGCAGAAGGGCCTTTAAACACAGGTTCATCAGTAGGGAATGAC |
| | CTTATATCCGTCCTTTTAGTTACAATCCCAAATGTAAGTTCATTCGATTCATTTATAGCCTCAAAATTCATATT |
| | GTCTAATAGTTCTTCATAAAAGGCGTCTTTTACAGGTTTTCCCTTCTTATCAAACATATTTTTATCTACTGGGA |
| | GAGGGTCTTCAAGCAGCCATTTTTCAAGCTCTTCTTTTGATACCGATGTTCCCATTTTTATTACATCTGCCATTT |
| | CATTGTTTTCGCCAAGAATCAAAGCGTTTAATTCTGCAATTTTCTCTGAAGAAAGTATTACCTCATCAGGATTA |
| | TCAAGCCTTCCAATCCAGAAGTCTGCTTTAAATATTGGAAGGTAGGCGCTTGCTTCGAGCAATACGGGAGAG |
| | AGAAAAATTATACTGATGACTATCTTCAAAACTTTCATGCCAACTTTGATAACATAATACGTCAATATTCTCA |
| | AGTCATAAATTATGTTCGGTAAAATCAATGTCTATTTTTGGGTATGTTTCAAGCTCTGGACAATAAAATACTA |
| | CTGTGATATACGGAGTCTGTGGAAAAGATTAGAAATCCTATTCCAACAGTTGATATAATAATTGAATTAAAA |
| | ATCCCCCCTCACCCCCCTTTTTCAAAGGGGGGAAGGGGGGATTGGGGGATTGTCCTTATAGAGAGGAAAAAT |
| | CCCCCGTTTGGCTGGGCAATTCCAGGCGGTTTTGTGGATTATGGAGAAACCTTGGAAGATGCGGCTATACGAG |
| | AAGCTCATGAAGAGACAGCCTTGAAGGTGAGACTAAAATGCCAGTTACATTCTTATTCAAACCCTACGAGGG |
| | ATCCCCGATTTCATACAATTTCCACAGTATTTGTTGCAGAGGCAGATGGAACACCGCAAGCCAGAGATGACG |
| | CAAAAGGAATCGGCGTTTTCACAGAAGACAATCTGCCTAAGCCTCTGGCCTTTGACCATAAGAAGATATTGG |
| | AAGATTATTTCAGATGGAAGAAGGAAGGGTGGAGGGTGTTTGAGGAAAAGGCATTGCAGTCAAATGATGGC |
| | TGTGATAACTTGCTTAGCTGAAAGGTAATTGGATTATGATAAAAGATACCCAACTATTAAAAAAATTTGAAG |
| | ACACAATCATGAAAAAAGAGGGAAGGCTTTCTTTTAGTTACTCTATGCGCATATTTGAATCTCTCTGGAATGA |
| | AGGGATAAAACTCGGAATATTGCCTCCCAAGAAACCATTAGAAGGAATAGAAGTTGATATAAAGATAGCAC |
| | AGGTGCTCAATTCATGTTTGAAGAAATCCTCGCAAGGATAGGCGCAGCGCTTGTTAAGTGCAATATTCCCTAC |
| | ATGATTATAGGCGGACAGGCTGTATTGCTGTATGGAGAACCGCGGCTCACAAGAGATATTGATATAACCCTC |
| | GGCGTTGATACAAATCACCTTAATCAACTTCTTGCAATCCTTCAAAAAATACCACTCAAGCCCATACCAGAAG |
| | ACATAGAATCATTCGTCAAGCAGACTATGGTCTTGCCGGCGCTGGATGAAACTACAGGCATACGGGTTGACT |
| | TTATATTCTCATTTACGCCTTATGAAACAGAGGCGATAAAGAGGGCAAAGAGTATAAATATTCTCAATCAGG |
| | AAGTATGCTTTG (SEQ ID NO: 44) |
| | GGACGATGTCGCTGGTATTCTCAAAGATCGCAGAAAGCAAAGAGGTATCAGAAGAACCGCTCAAACCAAGA |
| | AAAAGCGGCTGCGTGAATTAAAAAACTATCTAAAAAGTATTGGATATAACGAATCCACAGCAACATTTGAAA |
| | CGGTTTACAGCCTTGCCCATAAAAGAGGATATGATTATGCTGATATGCCTGAAGAAAAGACTTCCGAAGAAA |
| | TAGAGGCAATGGATGTGGAAGAGCGCAAACAGTGGGAAAAAGAGAAACAGGAATGGGAAGAGACAAAAAG |
| | AAATTCAAGGCACAGGAAAGAAGTCGTAAAAGATGTCCATAAAGCAATGATTGAAGGAAGAGCAACCGAGG |
| | AGCAGATAAAAAGGGTTGAAAGGATTTTTAATAAACAGTACCGACCTAAAAGATTCAATAATAGGATTTTAA |
| | CTAAATGTAAGGTAGAAGATTGCGGAGTTAATACGCCGCTCAGAAAGAATGTGAGAGATTTGCTTATAGAAA |
| | ATATAGTCAGATTTTTCCCTATAGAACAATCTGAAAAGGATAATCTCAAAGATGCTGTGCTTGATAAAAACAG |
| | GAGGGAAGAAGTTAAATCATTTTTCAGAAAACATAAGACTGATGAACATATTAGGAAGCAGGTTTATGATAT |
| | AGCGGATAACAAGCTTTCGGGTAGAACGGTCTTTTGCAAAGAACATATCCTTGAACATACTGAGCATAGCAA |
| | AGAGGAACGAAAGGTTTTCCGTCTTGCGCCATCCTTAAAAACAAAAATAGAGAATGTCCTTGCTGTTATTAAA |
| | GACGAGATATTGCCCAAATTTACTGTCAATAAGGTGGTAATGGAGAGCAACAATTTTGATATAGCAGCCAAA |
| | ACACAGGGGAAAAAACGATTGGCAAAAGAAGAATATGGTAAAGGGCCCAGGGAAGGCAAAGAGACCAGAA |
| | AGGAAGCCCTTCTTCGAGAAACGGATGGTAGATGTATCTACTGTGGAAAATCTATTGACATATCCAATGCGC |
| | ATGATGACCATATATTTCCCCGAAAGGCCGGGGGCTTAAATATCTTTGCAAATTTAGTAGCATGTTGCGCTGT |
| | ATGTAATGAGAATAAAAAAGGGAGAACTCCGTTAGAAAGTGGAATCTCGCCTAAACCCGAAATAATAGCCTT |
| | CATGAAGAATGATTTAAAGAAAAAGATTCTTGAGGACGCACGAAATATCAATACAGTAGATTTTAATAAATA |
| | CATGTCCCATGCCTCTATCGGCTGGAGATATATGAGGGACAGACTAAGAGAGTCTGCCGGCAATAAAAAGTT |
| | GCCCATAGAGAGGCAGAGCGGCATATACACTGCATATTTCAGGAGGTGGTGGGGATTTAAAAAGGAAAGAG |
| | GCAACACCCTTCATCATGCCCTTGATGCTGTAATCCTTGCTTCAAGGAAAGGGTATAGTGATGATGGATTGGT |
| | GGATATGACCCTTAAGCCAAAATATAATAAAGGTGGAGGGTTTGACTCTGAAAAACACCTTCCTGAACCGAT |
| | AGAATTTAAAAGGGATAAAGGCAGCAAGGGCTCTGCATTGCATGATAGAAACCCACTGTCTTATAAGAAGGG |
| | CATTATTACCAGAAGATTTATGGTTACAGAGATTGAGTGCGGTAAAGAAGACGATGTTATTTCCGAGACATAT |
| | CGTGAAAAACTAAAAGAGGCTTTTAAACGATTTGATACGAAAAAAGGAAAATGTTTAACAGACAAGGAGGC |
| | AAAGGAAGCAGGTTTTTGCATTAAGAAGAATGAATTGGTTATGAGCCTTAAATGCAGCATTAAAGGAACCGG |
| | GCCAGGGCAGATGATAAGAATCAATAATAATGTTTTTAAGACGAATGTCCATAATGTTGGCGTGGATGTTTAT |
| | CTTGATGAAAAAGGTAAGAAAAAAGCATATGAGCGAAAGAATCCGAGACTGTCAAAACACTTCATAGAACC |
| | ACCACCGCAGCCTAATGGGAGAGTTTCATTTACCTTAAAACGAAGAGATATGGTTACAGTTGAAGGTGAAGA |
| | CGCAATCTATAGAATAAAAAAACTCGGAACA (SEQ ID NO: 45) |
| | ATCAAAAATCCTCCGCATCCATCCGGAATATCAACCGGAGGAAAAACTCCCGCCAGCACAGTTCAATCTCTTT |
| | ACAGGAGAAGCAGAGCCAGTCCCGCAACTGATTGACAAAAATATTGATGATGAAATATTTGTTGACGATGGG |
| | ATTTCGCTTGTGCAGAGCGGAGATTCGTCGCAAATAGTTCTGTCAGGTTTTGGCATCTTCTTAAGTAAAAAGA |
| | GTGAACGCCTTATTGTAAAAAAGAGCAGTAAGGTTATTTATGAATTTCCCTTTTTTAGACTGAATGAGGTTAT |
| | TGTCGCCTCTCGTGGGATGTCATTATCCTCTGACCTTGTTGAAGAGTTATGCCAAAGGGGGATAAGACTGAAT |
| | TTTTTGACAAATGGCGGCAAACCGTATGCAATGCTTTCCTCGCCCATGCTCTCCGCAACGGTTGTATCCAGAA |
| | GAGAGCAGATTATGGCGTTTAATGATGAAAGGGGGCTTGAATTCTCAAAGGCGGTTGTAGAAGGAAAGATTG |
| | TAAATCAGGAAAGGCTTATAAGATATTTTGGCAAGTATATTAAGACCGTAGATATTGACAGATTTAATAAACT |
| | TGAAGGAATTGCTGATGATATTAAAAAGATGAGCGGACAGGTAGATAATATTGATGGGCAAAATATTGAAGA |
| | TGCAAGAGGTGGTTTGATGGCAATAGAGGGTATATCAGGCAGGTTATATTGGGGCGCGGTCAAAGAGATTAT |
| | TGCAGGAAAGGTTGAGTTTTTCGGCCGTGAGACAAGAGGGGCAGTGGATAAGGTAAACTCTCTCTTAAATTA |
| | TGGTTATGGCATTCTCTATTCGCATGTCTGGGGAGCAATTATAAATGCAGGTCTTGAGCCATTTGCGGGATTT |
| | CTGCATGTGGACAGGCCCGGCAAGCCGTCTCTTGTTCTGGATCTGATTGAAGAATTCCGCCAGCCTGTTGTTG |
| | ATAAGGTTGTAATTGCCCATATCAACCTCGGAGAATCCATTAACATGAAAAATGGATTGATTGAAGAGGAAA |
| | CAAGAAAGACAATGGGAAGCAAAATTATAGAAAGGCTGGAAAGCCAGGAAACATTTGAAGGCAAGAAATAT |
| | AAA (SEQ ID NO: 46) |
| | GAGACGATTGAATCTTATATTAAGACTAAAAAGATTCCTCCTTTCAATGTGAGCAGCCCGGCGCTTCTGGAAC |
| | ATCGTGGGGCATTTGTTTCTATAAAGACAAAGGGCAATTTGAGGGGGTGTATAGGCATCTTTGCGTCAGAAA |
| | AACCCCTTTATCTCACTGTTGTAGACATGGCTGTTGCCACTGCTACGCAGGACCCCAGATTTATTCCGCTTACT |
| | GCAACTGAACTATCACTGATTACCATAGAGATATCTTGTCTTACGGCGCTAAAAAAGGTAAAAGATACTACT |
| | GAAATAGAGGTTGGGAGGGATGGTCTGTATATTGTAAAGGGGTATTGCAGAGGCGTTCTTCTCCCGCAGGTC |
| | GCAGTAGAGTGTTGCTGGGACAAAGAAACATTTTTAGAACATACTTGCCTTAAGGCAGGTCTTCCATCTGACG |
| | GCTGGAAGGATGGGGCTGATATATATACATTTGAGGCAGAGGTGTTTGGCGAAGGCGATTGATAAGGCCGTA |
| | AAATAGCGATTAAAAACTTCGCTATTTTACAAACCTGCGATTTGCCTGCTCCCCGATAGAGACACTCGGGGAC |
| | AGGCATACTTCGTTATCGACACGCTCAGCGTGCCTTGACCAAGCTTAGCTTGGTCGGCGCATTTTTGCTCCTCA |
| | CCGTATCCTCCCCCTGTCCCCCTGTGGGGACAGATTTCAAATCTGTCCCCAGAAAAGGGGGAGGGCTCGTCGC |
| | AAAAACATGTCCCTGAGTGCTTTTATCAGGGATGGCCTCTGCCTCGTCTGACAACCCCCGATAGAATCAATCG |
| | GGGGCGGTTTCCCCAAAAATCAAACGCTATTTTTGGGTAAGGTATTTTTAGTGAAAAATATTCTTACTTTATG |
| | CTAAAATAAAACCCCAAATTTGTATACGAATGTTAAAAAATAATTCTACAACAATCCATGAGACTGATTAAA |
| | TCTATACTTATTATTTTAATTATCTGTTTAATAACTTCCATTTTTAGGACAAATGCAGAGGCTATACCAGCCTT |
| | TGCAAGAAAGTATAAGACGAGCTGCACAACATGTCATGCAGCTTTTCCAAAACTTAATGCCTTTGGTGAGGC |
| | GTTCAGAAGAAACGGATACCAGATCCCAGAGGTTGACGAACGCTATGTTAAGGAGAAACCTGTCAGCTTGGG |
| | CGCTCCGGCGTGGAAAGAGGTTTGGCCTGAAGGAATCTGGCCAGGCGAGATTCCTGGGGCAATACCCCTTTC |
| | TTTATTTGGAAGTTTGTTTTACCGATATGATGTAAACTCTCGGGTAAAGCATGATTTTACCTTTCCAAGTACCA |
| | TAGCGCTTGAATCAGCTGGAACACTCGGTGAAGATGTATCTTTTTTTGGCAGTGTTTCTCTTATTAGAAGTGG |
| | AAATGAGTTCGGCGGTTTGTGGCGTTTTTTCCTGAAGTTTAATGATACATTCAGCAGCTCGCTTCCTGACCGCC |
| | TTTTGAATGTGACTATCGGTCAATTCGAACCGTCAGCAGTCCCGGTCTCCAATAATCGCCGTTTAACATATGC |
| | GCCATATCTCATTAATACGTTTACAGTCGGGAAAAACAGCTTTAACTTCTCAAATCAGCGAGGCATAGAGATA |
| | GATGGCATTATAGAGAGCCGCTTAGAATATGCAGTGGGCATAGTTAATGGCAACGGTACAGGGAAGACAGAT |
| | TCCACAACCAACTCTGCAGACAATAATACGACAAAGGACGCCTGTCTTAAGGTTGGATATAAGTTCGGCGGC |
| | ATTGGCCTGGATGGTTCTGGCCTTATCTCAGACAAAGAAGATGTCCTGCCAGAGGTAAAGGATGAAAAATCG |
| | CTATATGTTGGCGTAATAGGTTATTCAGGCAAAAATAAAGTGGTTGTTTCAAACAGCACGTATGACGATAATT |
| | TCTATAGGTATGGCTTTACCTTTGACTTCAATTATAAAATTTTTAATCTGTTTGGCGCGGTTATTCAGGGGGAG |
| | CATAGTAATCCGAGTGGCAATTTTAGTGATACCTCTGTAAGTTCCTATTTTTCTGAAGCCGACCTTGCCATTTA |
| | TCCTTGGCTTATTGCAGCGGTGAGGTATGGAGTAGCTGATATAGAACATGAGAAGGAAAATAAAGATGAAGT |
| | GGTTGTAAGCTTGTTGGCGCTGGTTCGCGCAAATATAAAGCTTACTATCGAAGGGGCGCTGCATACCACAGGT |
| | GGCGAGGGGGACCTGGGCATAGTTAAACTTGATTTTGTCTTTTAAGCCAACATTTTCTTATGCCAATTCGCTCT |
| | GCGAGAATGCCGTGTAAACCCCGTTAGAGAACTATGTTTCCTAACGACCAAAGATAGTGGCAGCGCCCACCG |
| | TAAGACCTCTGGTCTCTTACGGGGTGAACTACCGTCTCTAAGGAATGATTTATACTACCGTCCGCCTCAGGCG |
| | GAGGAGTCGGAGGCTTTCTCTAACGGGGTAAAATATACTTGTAGCGCCATACAATGACAACACCTCAATTTTT |
| | CAATAATACTTTCTCATAGACCTGGGGGAAGCATCTATGATTTTAATAGACATAAGCAAGAAACTGTTTAACT |
| | CAAAAAATGCAGTTGCATTTTTTGACAGGAGTGGTAAGTATGCGATTGTGCCGTTGCAGATGAAAGCGCTTCT |
| | GCGGTTGAGATATTTAGCCGCATGCGCAATAGTGCTTATTTTGAGCTGCATATCGCATGCCGAGACTATTAGA |
| | GGAACCGTCAAGCTAAAGAGTGAAGATGATGCGGGGAAAGTAGTTATTTATGTCGAAAAAGCTCCAGGGGG |
| | ATTTATACCGCCAGAGAAGCATCCGGTTATGGACCAGATTGAACTGATATTTGTTCCGTATGTTCTTCCTATTC |
| | TCATAGGAACTACTGTTGATTTTCATAATAGCGATGATGTGCTCAATAATATCTTTACTCCGTCATGGGCAGG |
| | GCACAAATTTAATCTCGGCACCTATCCCAAGGGAGTAGTGAGATCATTTACATTTGACCGTCTCGGAGAGGTA |
| | GCATTATTATGCAACATCCATCCTGATATGGAGGGCTATATACTGGTTTTGCAGAATCCGTATTTTGCTATCCC |
| | TGACAAGCAGGGGAGGTATGAGATAAAGGATGCGCCTCCGGGGGTTTATGATTTCAAAATGTGGTATAAGAG |
| | GAAAGTAAGCCCTTCTTATACGGTGACTGTTGAAAAGGGGAAAGATGCTATAGTGGATTTTAAATAAGGGTA |
| | ACGGGAGGTAACGACGTTGAAGGTGACAATAAGGGCAAAATTAATGATTGGGTATATAGCAGTCCTAATCTT |
| | TATGATTATGGTGAGTATCTTCGCATATATCACACTTGTTAATGTCAACAAAACAGTAAATCAAATACTTGTT |
| | TCTGCCCATAAATATGACACGGTTAACAGCTTGCGGCAGTCTGTTAAACAATTTGTTGATGTCAGTGATTCGT |
| | TAGTAAGAGGGCAGATTCAGGATGTGGGTTATTATCGCTCTCTAACGCTTGATGTGAAAAAAAAGCTTTTGTA |
| | TGTCCGTAAACTGCGACTCAAGGAGCATGAAAAGGATTTCTTGGAAAAAGTAGATGCTGAATTTGACTCTAT |
| | ACAAAATCTAACAGAGCAATCCTTAAAGTGGACAGCAGCTTTGCGGAACACAAATCTCGTTGGCGTGCTCAA |
| | GGAATTGGACAAGGCCAAGCCAGTTATAATCAGCAGTGTTGATGGACTGTATGATGAGGCATGGCGCTCACT |
| | GGATAACGTTACTATTATGACTAACGATAATATGAGGAAGGGCCTGCAACAGATTCTGACATTTTCGATAATC |
| | GCTATTTTGGCGGGAATAGGAATATCTATTTATATTTCTCAAAAGATTACTACGCCAATAAGGGCATTATCCA |
| | TGTCCGCTGCCAGTGTTGCGAAAGGCGATTTGGATAAAATGGTGGAGAAGTCGTCTGCAGATGAAATCGGGG |
| | AATTAGTTGCTTCGTTCAATCAGATGCTGGTTGAATTAAAAAGCTCAAGGGAACAGATTGAAAAGTATAACA |
| | AAGAGCTTGAGACCATGGTGGATGAAAGGACAGCAGAGCTGGAAAAGACAAAGGAATACCTTGAAAATATT |
| | CTTGAACATTCGGGAGATATGATTATCACAACAACCCTCTATGATGAGATAGTGCAGTTTAACAGAGGGGCT |
| | GAAAACATACTGGGCTATGTCAAGGAAGACCTTGGCGGAATCAAGATAGGAGATATTTTTGTCAATAAGAGT |
| | GAGTATAAGCGGATAAGAAAGAAGGTAATCGAGGACGGAGAGATATCCGGTTATGATGCGCAGCTTATGAA |
| | GAAAAGCGGTGATATCATACGCGTAAGTCTTACGCTCTCGCGTCTTGAGGACAGGACTGGCAATATTATCGGT |
| | CTTGTAGGCATAGGCAGGAATATAGGCGAGGATAAAAAAAGTATTTAAATGAAGATACATAAAAAGGGGAT |
| | TCAACGCAGGATAGCCATTTTTATATTTGTCGTAGGGATGTTTCCTGTCATGATAAGCATACTTTTAGTTTATC |
| | TGCAGGGGGTGAGGGAGCTTAGAGTAAGTGCGGGTGAAAATTTTGCGCGAGTAGCTAAAGAGATAGCGAGT |
| | AACGCCGAAATTGTTATGGAGCAATCTGTTCACGATGTTAAAAGCCTTGCTATTTCACCGGTCTTAAGAAATG |
| | CAGCGATATTAGCAAATGAATCCTATACAGGTAAAGATAAGGGGTCAATAGAGAGGCATATCCAGAAAATTG |
| | ATATGCAGTGGTTGCAGCATGGAGACAAGGAAAAACGTTTTGGGGCGTATGTATCCAGTCGGGCTGCGCAAT |
| | ACTTAGCAGAAATAAAGAAAAATACAGAGGGATACGTTGAGCTTTTTATAACCGACACAAAAGGGGTATTAG |
| | TTGCCTCCGCAGGAAAGACAAAGTATTTATACTATGGCAGTGAGAAGTGGTGGCAGACAGCCTATAATAAAG |
| | GTAAAGGAGATATTTACATAAGCGAGATATACCTTGACCCTGATATCAATCAGTTCCTACAAAGTGTTGCAGT |
| | GCCAATTATGTCTGAGAAAGAAGGAAAGGTGCTGGGAGTTATTCGTGCAGTTAGTACCATAGATAAGATTTC |
| | GAAGATAGTGCAGAAATTCAGAATTGGAAAAACAGGCCATGCAATGCTTATAAATTCCGTTGGGATTATCCT |
| | CCTGTGTCCTATATTCCCGCCTCAGGTTCACAGGGTTACTGACGAGCTGATGAATAATATTACCACAGCGCCG |
| | TTACTTGGCTGGGCGGTTGTTAAGGATAATGCCCATGAAGGCATTAATGCTGTTGCGGGATTTGCCCCTATTG |
| | CAACGACCGATATTGTAAAGAATGCCTTTGATGGAAACAAATGGTATATCTTTGTCAGTCAATCACCGGATGA |
| | AAGTTATGCCCCTATTTATGTTTTGCTAAAGAGGGTTTTTTTTCTACTGATATTCTCTATTTTAATACTCTCTGC |
| | TATGGGATTCTGGGCGGCAAGAAAGATTGTGAAGCCGATAGAGACGCTTCGGCAAGGGGTTGAGATAATTGG |
| | GGAAGGCAATTTAAACCACCGTATCCATATTAAGACACATGATGAGATAGAGGAGCTTGCAAATAAATTCAA |
| | TCAGATGGTGGAAAAAATTAACAAGACACATTCTGAACTTGAACAGCGTATTTCGGAGAGGACAGGGTTTTT |
| | GAGAAAAGGCTATCAAGAGATGGAAGTAATGAGCCGTCTTAAGTCAGAATTTCTCACAAATGTATCTCACGA |
| | GCTGAGGACGCCGCTTAATTCCATTTTGGGATTTTCAGAGCTGCTCCACGATAAGGTTTGTGGAGATCTGAAT |
| | GAAAAGCAATCTGAATATATAGGGTATATTTATCGGAGCGGTAAACATCTTCTCGATATAATAAATGACATCC |
| | TTGACCTGTCAAAGATTGAGGCAGGAAAGATGGAGATAAAACCAGAAGATTTTTTAGTTTCCAATGGAATAA |
| | AAGAGGCATGCGCTATAGTGACTCCTTTGGCTATAAAGAAGAAAATACCTATTGATGTTGAAATAGCACCTG |
| | ATGTTTATACTATCGTGGCTGATATAAGGATGTTTAAGCAGATAATGTATAATCTTATCAGCAATGCTGTGAA |
| | ATTTACCCAGGAAGGCGGCCATGTCAGCATTAAGGTTAAGTCCAACAACTATTATCTTCAGGTTTCGGTGATT |
| | GATACTGGTGTAGGTATCAAAAGGGAAGATATGGGCAGCATATTCAAAGAATTTAAACAGGTCTCAACCGGA |
| | GATCAGGAGGGAACTGGTCTTGGTCTTGTCCTTGTAAAAAGGTATGTAGAAATGCAGGGCGGTAGTATCAGG |
| | GTTGAGAGCGAATTTGGCAAGGGGAGTAACTTCACCTTCAGGCTTCCGATGAATATTACGAAAATATAAAGA |
| | GCAGGCTATGGGCAATGGGCTATAGGCTATGGGGTTTTTCCTATTGCCTATCGCCTCTACCCTACTGCCTACTT |
| | TTAATAGGCATATAAATTTCCATATGGTCGGAGGGAGGCAGGTGTCAGCTTCTTAAAACCGGATTTCATTATA |
| | TCTTCGGTTTTAAGGTTAAAGTCTTTGCAATAATCCTCTAAATGTTCCTTGAGAAATTTTTTGTCCTCTTCGTTT |
| | AAATCTGTAATTCCAACCTCAGCGCCGCACCCTTTTTTATCCACATCGCCTCTTATAAATATCACGCCGCCATG |
| | CATGCCTGTGCCAAGGTAATCCCCAACCAACGGCCTTTTATTGTCGCCATCAAGGCCCAGGAGGATTAATACG |
| | CCGCCTGCCATG (SEQ ID NO: 47) |
| | 1 cctctgtaga agatgtcata atacacaaaa tatttgcagg acggccgagg gatttagagg |
| | 61 atgcaaggac aatcctctta aaaaatcctg atattgacat tagatatata cagggttggt |
| | 121 taaaggagtt tgacgcatca attggcaata aggattttct gaaaaccttt gaggacatat |
| | 181 taaaaaccat aaatcagtag aataggcttg tttttagtgg taaaaggaag gacagaaggt |
| | 241 ttttgagata tggtgatccc catcgtcata atcaaaggta aataaaaatg gaaagagaac |
| | 301 ttgttttagg cattgattac ggcggcaaat ataccggtct tgcagttgtt gaccgaaggc |
| | 361 ataatcaggt attatatgca aacagactga agatgcgtga tgatgttgca ggtattctga |
| | 421 aagatcgtcg taaacagcgt ggtattcgtc gtaccgcaca gaccaaaaaa aagcgtctgc |
| | 481 gtgaactgaa aaactatctg aaaagcatcg gctataatga aagcaccgca acctttgaaa |
| | 541 ccgtttatag cctggcacat aaacgcggtt atgattatgc agatatgccg gaagaaaaaa |
| | 601 ccagcgaaga aattgaagca atggatgtgg aagaacgtaa acagtgggag aaagaaaaac |
| | 661 aagaatggga agaaaccaaa cgcaatagcc gtcatcgtaa agaagttgtg aaagacgttc |
| | 721 ataaagccat gattgaaggt cgtgcaaccg aagaacaaat taaacgtgtt gaacgcatct |
| | 781 ttaacaaaca gtatcgtccg aaacgtttta acaatcgcat tctgaccaaa tgcaaagtgg |
| | 841 aagattgtgg tgttaataca ccgctgcgta aaaatgttcg tgatctgctg attgaaaaca |
| | 901 tcgtgcgttt ttttccgatt gagcagagcg aaaaagataa cctgaaagat gcagtgctgg |
| | 961 ataaaaatcg tcgtgaagaa gtgaaatcct tcttccgcaa acacaaaacc gatgaacata |
| | 1021 ttcgcaaaca ggtgtatgat attgccgata ataaactgag cggtcgtacc gttttttgca |
| | 1081 aagaacatat cctggaacat accgaacaca gcaaagaaga acgcaaagtt tttcgtctgg |
| | 1141 caccgagcct gaaaaccaaa attgaaaatg ttctggccgt gatcaaagat gaaatcctgc |
| | 1201 cgaaattcac cgtgaataaa gttgttatgg aaagcaacaa cttcgatatt gcagcaaaaa |
| | 1261 cccagggtaa aaaacgtctg gcaaaagaag aatacggtaa aggtccgcgt gaaggtaaag |
| | 1321 aaacccgcaa agaagcactg ctgcgcgaaa ccgatggtcg ttgtatttat tgtggtaaga |
| | 1381 gcattgatat tagcaacgcc catgatgatc atatctttcc gcgtaaagcc ggtggtctga |
| | 1441 atatctttgc caatctggtt gcatgttgtg ccgtttgcaa tgagaacaaa aaaggtcgca |
| | 1501 caccgctgga aagcggtatt agcccgaaac cggaaattat tgccttcatg aaaaatgacc |
| | 1561 tgaaaaaaaa gatccttgag gacgcacgta acattaacac cgtggatttc aacaaatata |
| | 1621 tgagccatgc aagcattggt tggcgttata tgcgtgatcg cctgcgtgaa agcgcaggta |
| | 1681 acaaaaaact gccgattgaa cgtcagagcg gtatttatac cgcatatttt cgtcgttggt |
| | 1741 ggggcttcaa aaaagaacgt ggtaataccc tgcatcatgc actggatgca gttattctgg |
| | 1801 caagccgtaa aggttatagt gatgatggtc tggttgatat gaccctgaaa ccgaaatata |
| | 1861 acaaaggtgg tggtttcgac agcgaaaaac atctgccgga accgattgaa tttaaacgtg |
| | 1921 ataaaggtag caaaggcagc gcactgcatg atcgtaatcc gctgagctat aaaaagggta |
| | 1981 ttattacccg tcgttttatg gtgaccgaaa ttgaatgtgg taaagaggat gacgttatca |
| | 2041 gcgaaaccta tcgcgaaaaa ctgaaagaag ccttcaaacg cttcgatacc aaaaaaggca |
| | 2101 aatgcctgac cgataaagaa gcgaaagaag caggtttttg catcaagaaa aatgagctgg |
| | 2161 tgatgagtct gaagtgtagc attaaaggca ccggtccggg tcagatgatt cgtattaaca |
| | 2221 ataacgtgtt caagaccaac gtgcataatg ttggtgttga tgtgtatctg gatgagaagg |
| | 2281 gtaagaaaaa agcctacgaa cgtaaaaatc cgcgtctgag caaacatttt attgaaccgc |
| | 2341 ctccgcagcc gaatggtcgt gttagcttta cactgaaacg tcgtgatatg gttaccgttg |
| | 2401 aaggtgaaga tgcaatctac cgtattaaga aactgggcac ctctcctacg atagaagcag |
| | 2461 ttgttggtag tgatggaaaa acaagaactg tctctgccac caagctgaca aaagcaaaca |
| | 2521 gtgctgaata gtttttaaaa ttaaatgtcc taacccacga tgaaggcgcc atttctggca |
| | 2581 tagaggctca ggctggacaa acccaagcgt atatcccttc actgtatagg cgttttacaa |
| | 2641 ttaaggctct tttaaaattt cgcaaacccc ttaaggtttg tttcaaaaaa tggtcttgcg |
| | 2701 gaaattttat aaattgtttt tgaaaatccc tttgaaattc cagcaaaaca atagctgttt |
| | 2761 cacgccatat tttcaaagag ccttaactgt aacttcgatc ttctcgaagg agatatgcgt |
| | 2821 gggcagatgt ttcacgccat attttcaaag agccttaact gtaactgtgt gaagcggtgc |
| | 2881 aaggcgcatc ccgcctttat aagtttcacg ccatattttc aaagagcctt aactgtaact |
| | 2941 ccttcttata cctgcggtag gccgcctgct cagaagtttc acgccatatt ttcaaagagc |
| | 3001 cttaactgta acgttcctca atctgtgcgg aaggccgccc cgaaagttgt ttcacgccat |
| | 3061 attttcaaag agccttaact gtaacgagaa gtcatttaat aaggccactg ttaaaaagct |
| | 3121 tgtttcacgc catattttca aagagcctta actgtaactt cataatacca cactgaaacg |
| | 3181 aaactcatgc taatgatact tcggctgtct tgcctgtaga ttcattaata acaaggagtt |
| | 3241 taccctcatc ctcttttttc aactgttcat tggcattcag caattctatg ccatagaccg |
| | 3301 ttccatcagg cgccaaatct atgttcagtt catcgctgat tttcacagtc tccacttgtg |
| | 3361 ctggtttttc atgcaggcga atataagcta tgttgtatct cggatcatat gttaacttca |
| | 3421 tatatgttcc accgtaaggc ttgcaatatg gcgtatactg ctttatagtt acaccacata |
| | 3481 acctcaaaaa tcaagaaaat attgaggagg tctatatgga taatgaaaca atcaggtcta |
| | 3541 ttatccagat gcaggcaaga aacctatcgg cttttttaag cggcaggaga gaatataagg |
| | 3601 cgttcaggtt taagtggtaa ttagttcacg ctgaaaaatg cccatctgct gcgttgcctt |
| | 3661 cgtcgctgcg ctgctcacat acctaaaagc gtatgctccg ctgctcgctc ctcggcgcct |
| | 3721 tgcatctgtg caatttttga gcgtgaacta tataaaagca tttttcaact aattatggga |
| | 3781 gaacatactg tttatatttt ctatgacata gaagacgacg gcatccgcaa taaggtggca |
| | 3841 gagacctgca aagactatgg gcttcaaagg attcaattta gcggattttc aggcacgttg |
| | 3901 ggcaaaaata agcgcgaaga acttttttta aagctttcct ttctcattgg cgataaagcc |
| | 3961 ggaaaactcc ttatgctccc tgtttgtgaa aaagacatca atgccaaaag ggaactcata |
| | 4021 caggaaaata gagatgattc atctcagggt tagcgacctt aaacagtttg tctattgtca |
| | 4081 aagggttgtg ttctataact atgttatgcc tgttgagaaa aaggcaacct ttaagatgga |
| | 4141 atatggaaag atagcagagg ataagataga cagattagaa aacagaagga aacttaagga |
| | 4201 atacggctta tccagcggca cgagagaatt tcataagcaa ttatattctg agagatatgc |
| | 4261 tttatccggc aaggtggatt tgctaattaa aaccaaagat tcttattatc ctgtggattt |
| | 4321 taaatataca acctctcagc cgcacaaaaa tcatctttat cagcttttgg gttatgctat |
| | 4381 aattttggaa gatgtatata atcgcaatgt tgataagggt tttgtatact taattccaaa |
| | 4441 agaggatgct gtggtttttg atttaaccgg caaagtcaaa gaggaaacaa aaaatttatt |
| | 4501 agacgatatc cgaaaaatga tacactgcca gcaaattccg cctccggtaa attctccggc |
| | 4561 caaatgtctt gactgcgaat atcgtaattt ttgcggagat gtattataat ccgaaatagt |
| | 4621 attatgcccc tttcaaaaga agataaattg ctcctcttaa aaatagtaag g |
| | (SEQ ID NO: 48) |
| 0209542_100 | GGACGATGTCGCTGATATTCTCAAAGGCCGCAGAGAGCAAAGAGGTATCAGAAGAACCGCTCAAACCAAGA |
| 61519_ | AGAAACGTTTGAGAGAACTCAGAAACTATCTAAAAAGTATTGGATATAACGAATCCACAGCAACATTTAAAA |
| organized | CGGTTTACAGCCTTGCCCATAAGAGAGGATACGATTATGCTGATATGCCTGAAGAAAAGACTTCTGAAAAGA |
| | TAGAGGCAATGGATGAGGAAGAACGGAAACAGTGGGAAAAAGAGAAACAGGAATGGGAAGAGACAAAAAG |
| | AAATTCAAGGCACAGGGAAGAAGTTTTAAAAGATGTCCGTAAAGCAATGACTGAAGGAAGAGCAGCCGAGG |
| | AGCAGATAAAAAGGGTTGAAAGTATTTTTAATAAACAGTACAGGCCAAAGAGATTCAACAACAGAATTTTGA |
| | CCAAGTGCAAGGTCTGCGGAAAAAATACGCCATTAAGAAGAAAAGTGAGGGAGTTGCTTCTTGAAAATATAG |
| | TTCGCTTCCTGCCGTTAGAAAGCGAAATGAAAGAGACTCTTAAACAGACAATTCTTGAAGGACAGCAGGAAA |
| | ATATCAATAAAATATTCAGGAAATTGAAATTTAATCAGAAAGACTGGCCGGGCAAGAATCTTACGGACATAG |
| | CAAAGAACAAACTGCCGGGCAGACTGCCTTTTTGCAAGGAACATTTCTCCGAGAATGAGAAGTTCACCATGA |
| | TTGAAAAATCAACTTTCCGTCTTGCACCATCCTTAAAAACAAAAATAGAGAATGTCCTTGCTGTTATTAAAGA |
| | CGAGATATTGCCCAAATTTACTATCAATAAGGTGGTAATGGAGAGCAACAATTTTGATATAGCCGCCAAAAC |
| | ACAGGGAAAAAAGCGATTGGCAAAGGAAGAATATGGCAAAGGGCCCAGGGAAGGCAAAGAGACCAGAAAG |
| | GAAGCCCTTCTTCGAGAAACGAATGGTAGATGTATCTACTGTGGAAAGTCTATTGACATATCCAATGCGCATG |
| | ATGACCATATATTTCCCCGAAAGTCCGGGGGCTTAAATATCTTTGCAAATTTAGTAGCATGTTGCGCTGTATG |
| | CAATGAGAATAAAAAAGGGAGAACTCCGTTAGAAAGTGGAATCCTGCCTAAACCCGACGTTATACGTTTTAT |
| | GGAGGATGAATTTAACAGACTTAAAATTAAGATGTGGAAGATCAGGCGAAAGCAGTCATTGACAACTGAAG |
| | AGATTATGGATAAGGCAAAGCGCAATAAGCTTGAACTGAAACTAAAAATCCTTGAAGATGCGCAGCAGGTCA |
| | ATGAACTCGATTTCAACAAGTATATGTCTCATGCCTCTATCGGCTGGAGATATATGAGGGACAGACTGAGAG |
| | AGTCTGCCGGCAATAAAAAGTTGCCTATAGAGAGGCAAAGCGGCATATACACTGCATATTTCAGAAGATGGT |
| | GGGGATTTAAAAAGGAAAGAGGCAACACCCTTCATCATGCCCTTGATGCTGTAATCCTTGCTTCAAGGAAAG |
| | GGTATAGTGATGATGGATTGGTGGATATGACCCTTAAGCCAAAATATAACAAAGGTGGAGAGTTTGACCCTG |
| | AAAAACACATTCCTGAACCGATAGAATTTAAAAGGGATAAAGGCAGCAAGGGCTCTGCATTGCATGATAGAA |
| | ACCCACTGTCTTATAAGAAGGGCATTATTACCAGAAGATTTATGGTTACAGAGATTGAGTGCGGTAAAGAAG |
| | ACGATGTTATTTCCGAGACATATCGTGAAAAGCTAAAAGAGGCTTTTAAATTATTTGATACGAAAAAAGGGA |
| | AATGTTTAACAGACAAGGAGGCAAAGGAAGCAGGTTTTTGCATTAAGAAGCATGAATTGGTTATGAGCCTTA |
| | AATGCAGCATTAAAGGAACCGGGCCAGGGCAGATGATAAGAATCAATAATAATGTTTTTAAGACGAATGTCC |
| | ATAATGTTGGCGTGGATGTTTATCTTGATGAAAAAGGTAAGAAAAAGGCATATGAGCGAAAGAATCCGAGAC |
| | TGTCAAAACACTTCATAGAACCACCACCGCAGCCTAATGGGAGAGTTTCATTTACCTTAAAACGA (SEQ ID |
| | NO: 49) |
| | 1 gcttgcaatg aaatttaatg ctacggatac taacacagaa ctattgaaca ttacaaacct |
| | 61 taaacccctt aagcccgggc cgtacatttt ccggcaaatt gcggccttgc acagcggata |
| | 121 taaaacgaaa aaactttact gcctttcttg ctttcaatca tggaagttgt tataaagatg |
| | 181 gttgcgaaga tggcaggaat gaaagaaggt agcaggcagg gaagggtgtg gtatgatggc |
| | 241 tgcaaatttg acaatttcta attttaatag aaccgttgtc ccattaacca tcttgctaaa |
| | 301 aaccgatggg agaggaaaga gagtgtttga ggtttagcat gaggaggttt tatggaaaga |
| | 361 gaaattgttt taggcattga ttacggtggc aaatataccg gtcttgcggt tgttgaccga |
| | 421 aggcataatc aggtattata tgcaaacaga gtgaagatgc gtgatgatgt tgccgatatt |
| | 481 ctgaaaggtc gtcgtgaaca gcgtggtatt cgtcgtaccg cacagaccaa aaaaaagcgt |
| | 541 ctgcgtgaac tgcgtaatta tctgaaaagc attggctata atgaaagcac cgcaaccttt |
| | 601 aaaaccgttt atagcctggc acataaacgc ggttatgatt atgcagatat gccggaagaa |
| | 661 aaaaccagcg aaaaaattga agccatggat gaagaggaac gtaaacagtg ggaaaaagaa |
| | 721 aaacaagagt gggaagaaac caaacgcaat agccgtcatc gtgaagaggt tctgaaagat |
| | 781 gttcgtaaag caatgaccga aggtcgtgca gccgaagaac aaattaaacg tgttgaaagc |
| | 841 atctttaaca agcagtatcg tccgaaacgt tttaacaatc gcattctgac caaatgcaaa |
| | 901 gtgtgcggta aaaatacacc gctgcgtcgt aaagtgcgcg aactgctgct ggaaaatatt |
| | 961 gttcgttttc tgccgctgga aagcgaaatg aaagaaaccc tgaaacagac cattctggaa |
| | 1021 ggtcagcaag aaaacatcaa caaaatcttc cgcaagctga agttcaacca gaaagattgg |
| | 1081 cctggtaaaa acctgaccga tattgccaaa aacaaactgc ctggtcgtct gccgttttgc |
| | 1141 aaagaacatt ttagcgagaa cgagaaattc accatgatcg aaaaaagcac ctttcgtctg |
| | 1201 gcaccgagcc tgaaaaccaa aattgaaaat gtgctggccg tgatcaaaga tgaaattctg |
| | 1261 ccgaaattca cgatcaacaa agttgtgatg gaaagcaaca acttcgacat tgcagcaaaa |
| | 1321 acccagggta aaaaacgtct ggcaaaagaa gaatacggta aaggtccgcg tgaaggtaaa |
| | 1381 gaaacacgta aagaagcact gctgcgcgaa accaatggtc gttgtattta ttgtggtaag |
| | 1441 agcatcgata ttagcaacgc ccatgatgat catatctttc cgcgtaaaag cggtggtctg |
| | 1501 aacatttttg caaatctggt tgcatgttgt gccgtgtgca atgaaaacaa aaaaggtcgc |
| | 1561 acaccgctgg aatcaggtat cctgccgaaa ccggatgtta ttcgttttat ggaagatgag |
| | 1621 tttaaccgcc tgaaaatcaa gatgtggaaa attcgtcgca aacagagcct gaccaccgaa |
| | 1681 gaaatcatgg ataaagccaa acgtaacaag ctggaactga aactgaaaat cctggaagat |
| | 1741 gcacagcagg ttaatgaact ggatttcaac aaatatatga gccatgcaag cattggttgg |
| | 1801 cgttatatgc gtgatcgcct gcgtgaaagc gcaggtaaca aaaaactgcc gattgaacgt |
| | 1861 cagagcggta tttataccgc ctattttcgt cgttggtggg gcttcaaaaa agaacgtggt |
| | 1921 aataccctgc atcatgcact ggatgcagtt attctggcaa gccgtaaagg ttatagtgat |
| | 1981 gatggtctgg ttgatatgac actgaaaccg aaatataaca aaggtggcga atttgacccg |
| | 2041 gaaaaacata ttccggaacc gattgaattt aaacgcgata aaggtagcaa aggtagtgca |
| | 2101 ctgcatgatc gtaatccgct gagctataaa aagggtatta ttacccgtcg ctttatggtg |
| | 2161 accgaaattg aatgtggtaa agaggatgac gttatcagcg aaacctatcg cgaaaaactg |
| | 2221 aaagaagcct tcaaactgtt cgacaccaaa aaaggcaaat gcctgacaga taaagaagcc |
| | 2281 aaagaagcgg gtttctgcat caaaaaacat gaactggtta tgtccctgaa atgcagcatt |
| | 2341 aaaggcaccg gtccgggtca gatgattcgt attaacaata acgtgttcaa gaccaacgtg |
| | 2401 cataatgttg gtgttgatgt gtatctggat gagaagggta agaaaaaagc ctacgaacgt |
| | 2461 aaaaatccgc gtctgagcaa acattttatt gaaccgcctc cgcagccgaa tggtcgtgtt |
| | 2521 agctttacgc tgaaacgtag agatatggtt acagttgaag gtgaagacgc aatctataga |
| | 2581 ataaaaaaac tcggaacatc tcctacaata gaagcagttg ttggtagtga tggaaaaaca |
| | 2641 agaactgtct ctgccaccaa gctgacaaaa gcaaacaatg ctgaatagtt tttaaaatta |
| | 2701 aatgtcctaa cccacgatga aggcgccatt tctggcatag aggctcaggc tggacaaacc |
| | 2761 caagcgtata tatcttctcg tataggcgtt ttacaattaa agctcttttg aatttcgcaa |
| | 2821 acctctatgg gtttctttca aaaaatatct tttcggaaat tttgcgattt gcttctgaaa |
| | 2881 aaccctttga aactccagca aaacaaaagc ggtttcacgc catattttca aagagcctta |
| | 2941 actgtaacaa agtaagagtg ccggttgatt tgaaattcgt gccaaagttt cacgccatat |
| | 3001 tttcaaagag ccttaactgt aactatacat attatattca atttcctgca accattctgt |
| | 3061 ttcacgccat attttcaaag agccttaact gtaacctttg attgtctcaa tcagggtatc |
| | 3121 caatttaagg gtttcacgcc atattttcaa agagccttaa ctgtaacaat aagaagccgt |
| | 3181 cattgctgca agcgtgccgt cgccgtttca cgccatattt tcaaagagcc ttaactgtaa |
| | 3241 catgtattgc ctgcaacatt ctggatgatt ccaaattcgt ttcacgccat attttcaaag |
| | 3301 agccttaact gtaacgagaa gtcatttaat aaggccactg ttaaaaagcg tttcacgcca |
| | 3361 tattttcaaa gagccttaac tgtaacttca taataccata ctaaaacgaa actcatgcta |
| | 3421 atgatacttc ggctgtcttg cctgtagatt cattaataac aaggagttta ccctcatcct |
| | 3481 cttttttcaa ctgttcattg gcattcagca attctatgcc atagaccgtt ccatcaggcg |
| | 3541 ccaaatctat gttcatttca tcgctgattt tcacagtctc gacttgcgct ggtttttcat |
| | 3601 gcaggcgaat ataagctatg ttgtatctcg gatcatatgt taacttcata ctatacccaa |
| | 3661 actctgccgc gcctctttcc gccaatcgtt cctatgcgtg tatatgaaat tttaccatat |
| | 3721 acggcttcta tcaatatgtc ggaactgttg caatcgcaag acttgcaata tgatttgcac |
| | 3781 tgctttatag ttacaccaca taacctcaaa aatcaagaaa atattaagga gggcatatgg |
| | 3841 gtaatgataa ttcaaaaatc ctccgcattc atccggaata ccaaccagca gaaaaactcc |
| | 3901 cgcctgcaca gttcaatctc tttacaggag aagcagagcc aattccacaa ctgattgaca |
| | 3961 aaagtattga tgacgaaata ttcattgaca atgggatttc gcttgtgcag agcggagatt |
| | 4021 cctcacaaat agttctgtca ggttttggca tctttttaag caaaaagagc gaacagctta |
| | 4081 tcgtaaagaa gagcgacaag gtcatttatg aattttccct ttgacaaact tttttctacc |
| | 4141 tgcaataatt ggccttgccc caaaaataga cattgatttt tggggaaact gacaagttgc |
| | 4201 ctgccagacg aggcggcggc catttttgtg acgcaaccgt atgctcaccg atacggtgag |
| | 4261 gaacaaaaat tggccgataa cgaagtatgg caggtaaatc gccagtttgt aaaatagcgg |
| | 4321 gggttttaat cgctatttta cggcttgttt ttaaaattct attgtattgc aagcttatag |
| | 4381 atgatctcag aaaacaaaag actcaaaaac acacccctgc atcaaaccca taaagaactc |
| | 4441 aatgcgaggc tggttccttt tgcaggatgg gaaatgcctg tgcagtacat gggcgttata |
| | 4501 gaggaacatc ttaatgtccg caaggcctgc gggctttttg atgtaagcca catgggtgag |
| | 4561 atagagatat ccggccctaa ggcccttga (SEQ ID NO: 50) |
| MHEF010001 | CACAGACATTCTTGCAGGTCGCAGAGAACAAAGAAGTTTAAGAAGAACACTACAAACTAAGAAAAAACGTT |
| 35.1_ | TAAGAGAGCTCAAGAACTATCTTGAAAGTATTGGTGGTATATATGAGGAATCAAGTGGGACATTTACAATTG |
| organized | AACCTTTTAGAACAGTTTACAGTCTTGCCCATAAGAGGGGTTATGATTATGCCGACCTCCCTGAAGAAAAAAC |
| | TTCTGAAGAGATAGAGGCAATGGATGCTAAAGAACGAAAACAGTGGGAAAAAGAGAAAAAAGAATTGGAG |
| | GAAACGCAAAGGAATTCAAGACATAGAGACGAAGTTCTTAGAGATGTACGCAACGTAATGACTGAAGGTAA |
| | TCTATCAGAAGAGCAGATAATAAAGGTTGAATCTATTTTCAATAAGCAGTACCGCCATAAAAGGTTTAATAA |
| | CCGAATCCTGACTAAGTGCAAAGTCTGCGGTAAAAATACCCCATTAAGAATAAATGTTCGGGAATTGCTACTT |
| | GAAAATATTGTGCGTTATTTGCCTCTCCAGAATAAGGAAAGAGAATTGCTTAAATTGACTATCTTGAAAGGGC |
| | ATCAGCAGGATATAAATGAAATCTTTAAGCACTTCCGAAAAGTTTACAAGATAACTCTAAACCAAAAAGATT |
| | GGCCAGGGAAGAATCTTATTGATATAGCACGGAATCAATTGCGGGGCAGATTACTTTTCTGTAAAGTACATTT |
| | TCCCGAAAATGAAAAATATGTTTCTATTGAAAAAAAGACCTTCAGGCTTGCACCATCCTTAAAAACAAAGAT |
| | AGAAAATGTTCTGTCGGTTATCAAAGATGACATTTTGCCTAATTTCACTTTAAACAACGTTGTTATGGAAAGT |
| | AATAACTTTGATATAGCAGCTAAGACAAAGGGAAAGAAACGATTACTTAAAGAAGAATACAGTAAAGGACA |
| | CAGAGAGAGTGGAGAAACCAGAAAAGAAGCTCTTCTTAGAGAAACAGATAGCCGATGTATTTACTGTGGGA |
| | AAGGAATAGATTTATCTAATGCTCATGAAGACCATATTTTTCCGAGAAAGGCTGGAGGTATTAATATCTTTGG |
| | CAATTTAGTCGCATGTTGCAGCGTATGCAATGAGGAAAAAAGAGGAAGGACACCATTAGAAAGTGGCATTTT |
| | GCCAAAACCAGAAATTGTATCTTTCATAACCAATGACTTGAAGAAAAAGATTCTTGAAGATGCACAATATAT |
| | TAATACTTTAGATTTCAACAAATACATGTCTCATGCCTCTATTGGATGGCGACATATGAGAGATAGGCTTAGA |
| | GAATTAACTGGCAATAAAGAATTACTTATAAAGCGACAAAGTGGTATCTATACTGCATACTTTAGAAAGTGG |
| | TGGGGATTTATCAAAGAAAGGGGCAACCACGGACATCATGCACTTGATGCTGTAATTCTTGCTTCAAAGAAG |
| | AGCTATGCAGAAGATGGCAAAGTAGATATGACTATTAAACCATGTGGCGAAGATGGCAAAGAATTTGACATA |
| | GAAAGACACCTTTCTGAGATGAAAGAGTTCAGAAGAGATAAAGGGGGTAAAAGTGCTCCTCTTCACGATAGG |
| | AATCCTTTATCTTTTAAGAACGATATTATAACTCGCAGATTTATGGTAACTGAAATTGAGTGCGGGAAAGAAG |
| | CCGTTATTATTTCGGAGGAATATCGTAAAAAACTAACTGAGGCTTTTAAACGCTTTGGTATTGCTAAAGGGAA |
| | ATATTTGACAGATGAACAGGCAAAGGATGCTGGATTCTATTTAAGGAAAAATGGCGAAGGGGTGATGAGTCT |
| | TAAATGCGAGGTAAAAGGAACTGGCTATAATCAGATGATTAGAATAAAAAATAATATTTTTAAAACAAATGT |
| | TCATAATGTTGGCGTGGCAGTTTTTCTTGATGAAAAAGGCAAAAAAAGAGCTTGTGAACTCAAGAACCCAAG |
| | ACTGTCAAAGCATTTTGTAAAGCCAGCAGAGCAGGTTAAAGGAAAAGTTATATTCATCTTGAAACGTGGTAA |
| | TATGGTTACAGTT (SEQ ID NO: 51) |
| | CATTTGTTAAGATTCAGAACTGACATTGAAAATGCTAAAACCTTCAAAGAAGCCTCAGATTCTTTTTTAACTT |
| | TATTAAAACCAATATCTAAGGAGAAACCTGCTGATTTTATTCAAGAGACAAAAAAGCAAAGGATGACAAGGG |
| | AAGATGCCTACGGAGGATATGAAAGAAAGAGTGATAAATACAAGGGCAAAAAAAGAGGCGATATTTGGAAT |
| | GTTATTAAAAAGGCAGAAGATATACTGACTGGTAAGAAGATAAGTGAGTGGGAGCGCATCGGAATAGGTGA |
| | ACTCGAATGGTTCAAGCTTTTTTCAAAACTCTCAAGAGGAATTGCTAAAGGAATTGAACTTCACGACTTAGAA |
| | GCTTACACGGGATACAGTACAAAGGCGATCCATAAAATGATAGATGAACAGTGTGTATTAAAGGATGTGTTG |
| | GATATCTTTAATAAAAAGATTAAAACAGCATTAGCAAAGCAAAAACCTGATGACAGGACTTTCTTAAACACG |
| | ATCAAAGGAGTATGGGGTAAAAAGGCAGCATTGGTCTTAGAGAATTGTACTTATGAAGGGGAACGAGGCCGT |
| | AAAGTCTCTAAGGAGACTATAAAGGAGAGGCATAAAAGGATAAGAGAAGTTATTTACAGTGGGAAAATTGA |
| | AAAGGTTAATTCAGGGACTTACTACAGTAGAGACTTTAGAGTATTTCTATGCGTAATGGCTCGTGTTGTTTAT |
| | CACCTCTTGTTTGAAATTCCCTTTTTAGGCGACAAATTGAAAAGAGAAGCAATATCAGAGTTTAGAAATTTTG |
| | CAAAAAAATATGATATAAAACTCTCTAAATTATCTGACTTCCAATTTATAGAAAGCAAACCCACATCAAAAG |
| | GCTTACCCGACATAAAGCCTCATAAATAGCCATTCTTCCTAATAGCACGAAATAGATTCAATTCCGTTCCTTT |
| | CAGATCAATCAGAAATCCGACATAATACCAATATATGTAAAAAAAAGATTTTTGTTCCAATTTTGTTCCCGAA |
| | TCCGAAAAAAGGAGGTAAAAGAATAATGGGAAAACACACCTTTGAAGATACTTCAATAGTAGCATTCTTATC |
| | TCTCAAAAATTACAAGGTAACACCACAGAGAACCTATGACGGAAAAGTTGTCTTTATTGTAGAAGGCAAGGA |
| | TATTAACAGGGCATTGCAGGAGCTTTATGGCAATTCTCAAGTAGGTGTTTTGGATTTTATTAAGACTTTAAAA |
| | GCCTTACGCTCAAGTATCTTTGCACTCAAAGCAGGGGGTGAAAGATGAAATCTATACTTGATCAGAATTTAAT |
| | CACTGTATATTCATTCCCTTTTGATATATTTCCATCAAAGCTATTGAATGTCATTAATGTACTAAGTGAAGCCT |
| | TACATGTGGAGCCTGAAATTGTAGCAAGTGCAATGCTTACCATAATAAGTGGGACAATAGGCAACACTGTAA |
| | GAATATCCCCTAAACATGGTTTTGAAGTGGCACCCTTTATATGGCTGATTATCATTGCCTTAAGTGGTTATGGT |
| | AAATCCCCTGTGATTCAAACCCTTCTAAAGCACATCAATGAATTGCAGGCAAAGGCTTATAACGAATACCAA |
| | AAGCAGTTTCAAGAATATGAAAGAAGACTTAGAAAGGCTAAACAAGATGAAAGCATAGATATACCAGAGAA |
| | GCCAAAATTAAAACATCATGTTGTATCAGATTGTACGGTGGAGGCATTGGCTAATGTGTTTGAGAATGATAGC |
| | AGAGGGGTTATTAGCTATCAGGATGAAATAGCAAGTTTAATTTTAGGTCTTGACCAGTACAAAGTCAAAGGG |
| | AACGATAGACAGCATTATTTGGAACTATTTAACTGTGATAGCTGGAAGATAGACAGAAAATCAGGTGTCAAG |
| | TTTATCCACAACACAGGTACTTCTATTATTGGTGGGATACAGCCTAAAGTTATGCCTGACGTCTTCAAAGTAA |
| | ATTCTTTTGATGATGGGTTTCTACCCCGTTTCTTATTGCTCAATGCTGAAAACAGACCCATGAAGTTTAGCAGA |
| | CAAGCAATTATTGAAGAGATTATATCTTACTGGAGAGACCTATTAAATTGGTGCTATGCCATTCCATTAGAAC |
| | ATGATGATGATGGATTCATAAAACCAAAAGTTTTAACCCTGAGTAGCAAAGCCTTAGACATATGGGAACAAT |
| | TCTATAACGATTACGGCGATAAGATGCCTTTCCTTTCAGAAAGGGCAAGGGTCTTTATCCCGAAACTTATAGC |
| | CTATCACAGTCTTAAGTTTGCTGGAGTTTTACATGTCATAGAGGCATTCAGCAAAGGTATAACATCCATAAAC |
| | AACTTAATCGAGGATGAGACAATACATCACGCTATTGAATTAATAAAATTCTATGCTGGACAGACTATTAAG |
| | GCATTGAAACTGTATGAGAAACCAGAAGACACTCTAAATGAATTTCAGAAGAGACTCATTGAAACACTACGT |
| | AGCTTGAAGGGAGAGGTTAAAAACGGGAAATTACAGTTATCAAGAATTGTTGAGCTATTCAATCATGGCGTA |
| | CCTGAAGGTGTGAGTCATACTCCAGAAGGAATTAGCAATATGTTGGCTGATTTGGGGTTAACTACTGAAAAA |
| | AGCACGGGCAATTATTCATATCTTTTATGGGAATCTGAGAAGATACAAAAACTTTTTTCTAAAACAACCGTAA |
| | CTACTGTAACTACTGTAACTACTGTAACTGATAAATCAAATTCAGAAAACAACAAAGTTACTGAAGTTACGG |
| | ATGTTACCGTTAATTCTGATGAAGAAGTTATAGACCTTGAACATGAGGAGTTTGAAATAGTCAAATGAGATA |
| | CAAAGTCTTAGAAAGTTTCAAGATTAAAACGTTAAAAAAGGAAATGGAACTGCAAGCAGGGCAGGTTGTCAC |
| | TCTTCCTCATGACAAGGCTATCATTCTAATCAGTGAAGGTAAGATTACTCCCTTTGATGATACAGCTTATAGG |
| | GTCTACTCGGAGAATCTACATGCTTACCTTTGGGTTGTAGAAGATGATAAGGATATTGACCGTCTCAGAGAAC |
| | AAGGCATAAAAGAAGCTATCTACACAAGACAGGAAATAGAGAAACTAAAAGATATTGATAAAGACTCTCTC |
| | AAGGTAATCCACAGAGTTAAAGAGGTCTTTGAGTCATCAAAGGTCGTGGAGATAAAGGGCTGTGAAGACTGA |
| | GCTTGAAACACAGGAGATTGAGGCGATAGCCGATAAGGTTATCGAGAAAATGAAACCGTTGCTTGCTGGTAA |
| | TGGTAAGTCAGAGGATGATGTTATCTTTGATGTAGAGGGGTTAACTCAATATTTGAAGGTATCTAAGCAATGG |
| | ATTTACGAAAGAACACACCTGAAAAAGATAGCACACTTAAAAATAGATGGGCAATTGAGATTTAGGAAAAA |
| | GGATATTGATAAATGGCTTAGCTCTTATAATATCCCCGCTATAAACACACCTTGAAGGATTTTAAAGGCTATA |
| | AAGTAAGAGGAGGCCATAAGATTGTATGTCCGATAACCTATATAAGAAACCCTTAATAATAAACAATCTACA |
| | AACCACTGTGCTTTAAATTGTGCACTAAAAATAATTCTTGACTTTCTAACAATGTTAGTATATGATTTAGATAT |
| | GCAAACCTGGACACCTGAAGAAATAGAGCAATTCAGAAAAGACAACAAGCTAAGCAGAAGGGCTTTAGGTG |
| | AGCTTTTAGGTGTTACAGGCAATTGCATATATCAATGGGAAAGGGGGTTGAGAGAGCCGAGTAAAACAACAA |
| | AAATACTTTTATCAAGGATTGAGCAAGAACTAAAAGGAAAGAAAAAACAAAAAGGAAAGGTAGGTGAAAAG |
| | AAGCATGGCAAAAAGTAAGCATCGGGGGATATTCCTGAGAAACCGCATTTACTGGATTAGATATGCAGGGCT |
| | TGATGGTAAGATGGTTTATGAATCCTCAGGCAACGAAAAGTTTAAAGATGCTGAAGCCCTGCTTATCCAGCA |
| | GAAGCAATCTATCAAAGAAGGCAAGCAACCTGAGATTAGGAAAATCTCCAATTACACATTCAAAGAACTTTC |
| | TGAGAAATACCAATCATGGATTAACGGCAGACAGAAATCAGCCAGAACAAAAGGCTATCTCATCAAACAGTT |
| | GGTTGAAAGGTTCGGTAACTTTCCGATTAGGAGATTCAATACGGTGATTGTGGAACAATTACAGACAGACCTT |
| | ATGAATAGAGGATTAAAGAATAGTAGCTGTAATAAGGTCTTGAATGTTCTCAAACATATGTTTACAAAAGCT |
| | GTGGAATGGGAGATGGTAGAGTCTGAAACATTAAAGCGTACCAGAAAAGTAAAGCTATTAAGAGACGATGG |
| | GAAAAGGTTAAGGTATCTCTCTACAGAGGAGTGTCAAGCCTTAATCAATGCCTGTGATAATCACCTGAAGCCT |
| | ATTGTAGTAACAGCAGTGAATACAGGCATGAGAAAAGGTGAAATACTCTCTCTTAAATGGGATAATGTAGAC |
| | CTTAGACATGGCTTCATTCTGTTAGATGTAACAAAGAATGGAGAGAGAAGGGAAATACCTATTAACGATACT |
| | CTCAGGATAACCTTACAGGGTATTACAAGGCGGTTAGATATTCCTTATGTATTCTATGACCAAAATATAGGGA |
| | AAGCCTATCAGGATGTTAAGAGAAGCTTTAAAACAGCATTGAGAAGAACAGGGATAAGAGATTTTCATTTCC |
| | ATGACCTGAGACATACCTTTGCATCTCATCTTGTTATGGCAGGGGTAGATTTAACCACAGTGTCAAGGCTGTT |
| | AGGGCATAAAACTTTAACTATGACCTTGAGGTATGCTCATCTTGCACCTGCTCACATGGTTAAAGCTATGGAT |
| | ATCTTGGACAATACCTTAAATGGTAAGTTTAGCTCAGTAGGTGAACGTTTGCATGATGCAAACTATACAAAAA |
| | CTATACAATCGGCAGAGGCAAGGTAAGATGAAATCTCCTAAGTGCTTGAAAAATATGGTAGGCACGAGGGGT |
| | ATCGAACCCCTGACCTCTTCCGTGTCAAGGAAGCGCTCTACCACTGAGCTACGCGCCTGTAAGACTTTATAAA |
| | ATAACATTTTACCCATTACTCCTGTCAACCTTGAGTTTATATCTTTTAGTCTCTTTTGACAGTTAATCATCTCTT |
| | TTTATATACTTTC (SEQ ID NO: 52) |
| | 1 tggttaaaac tacctcatgt ttattataac aaatttgttt attttgtgtt gaagtatcca |
| | 61 ctgcacagga agaaaagggg aggtgttaat gctcactaat taggtaactt tttggagttt |
| | 121 agcagttcat catatttacc atcgagataa gtgcttgaaa aatatggtag gcacgagggg |
| | 181 tatcgaaccc ctgacctctt ccgtgtcaag gagagtctgt cgtatgtcga ggttttacga |
| | 241 atctttataa acctttgtaa atttttgttg caaaggattt ggggggagaa gggttttata |
| | 301 tccttttccc ttcttttttg actgtctact atacaaaaac tatacaatgg cattaaacaa |
| | 361 tacttatttt tcagaccccc ctacatcccc tttttcagaa gaggggtggg ggttaagtgc |
| | 421 ttaccccctt tctcgcatat gaaatcagtt ttatgggaaa ggctaaaatt gacctaatag |
| | 481 acgctgtcta atttaaaggt tcactttgag gatttcacct acaactattc atcttcagca |
| | 541 agttcaaaga gttaactatt tccgagagaa attaacaact attcttttgt aaactcatca |
| | 601 ataaatttat caacattatg tgtcttacgc tgtatcaact taaataaacg ccaaagttcc |
| | 661 cctggggctg ataactcttc aaagtcaaat tcatattcca agataaaatc tcttgacgct |
| | 721 aatgcaagct gtggacttga acttccctcc ttaatataaa ttactataga tttgttacca |
| | 781 aattcacaat aataaccatc tttcagtgtc gagatatcat cacgaatgaa ctgataaata |
| | 841 atatttggag ataggtcatt aataagacta ttccaatcta atttatttgc ttttgttttt |
| | 901 tcaattaagc tccgaagaaa ggtcatttct ttcataataa aaattcctct tttttagaaa |
| | 961 gtttgccaag caaaatagct accttatctg cgactgctat tgtatcaata ttgtctaaat |
| | 1021 caccatctaa taatcgtttt agtttctcca gttccctttt atgttctttc ccaaattgaa |
| | 1081 attcttccaa tgtgcttatg agtcttttta aatcggaaat ctgttttcta tcaaaaatat |
| | 1141 tatcttcatt tataagccgt agacacccat ctaactctgc ataaatttta tttttttttg |
| | 1201 aaccccattg tttataatca atgtatttta ttaaagtttt ctttacccca tgagcatata |
| | 1261 ttaataatat aatgctaact actaagctaa gaacacttgc ccaattagct atatagtcaa |
| | 1321 acatctccat tccattagtt aacgcattca atatatatga cagttaggaa atagacactt |
| | 1381 gattaccgat acaaatctac aatttattca taattcctgt caatgttaac ttcaggaaga |
| | 1441 agcaaaccgg aaacttttct tcctgaaggt gacaccctga ataattcgca aaatgtgagt |
| | 1501 agtactcttg gtattaaaca gttgttaagg tggaaacaat gcgaagtatg tgtagaacaa |
| | 1561 aacaaggata atgttttata agaaaagaag tgcgatatca tccagatgtt aatctttttc |
| | 1621 gccttttact tgctgccatg aattttgatt ctaaaaaatg acttttctaa caaataattc |
| | 1681 cttgtatttt taattggaaa gttgtaaaaa aatcttatac tttaacttga cttattatgg |
| | 1741 aaaaagaact tgttttaggt attgattacg gtggcaaata tactggtctt gcggttgtta |
| | 1801 accagaaaaa taatcaagta ctttatgcac gaacagtaaa gatgcgtgat gatgttaccg |
| | 1861 atattctggc aggtcgtcgt gaacagcgta gcctgcgtcg taccctgcag accaaaaaaa |
| | 1921 agcgtctgcg tgaactgaaa aactacctgg aaagcattgg tggcatttat gaagaaagca |
| | 1981 gcggcacctt taccattgaa ccgtttcgta ccgtttatag cctggcacat aaacgcggtt |
| | 2041 atgattatgc cgatctgccg gaagaaaaaa ccagcgaaga aattgaagca atggatgcca |
| | 2101 aagaacgtaa acagtgggag aaagaaaaaa aagagctgga agaaacccag cgtaatagcc |
| | 2161 gtcatcgtga tgaagttctg cgtgatgttc gtaatgttat gaccgaaggt aatctgagcg |
| | 2221 aagaacaaat cattaaggtc gagagcatct tcaacaaaca gtatcgtcat aaacgcttca |
| | 2281 acaatcgcat tctgaccaaa tgtaaagtgt gcggtaaaaa tacaccgctg cgtattaatg |
| | 2341 ttcgtgaact gctgctggaa aatattgttc gttatctgcc gctgcagaat aaagaacgcg |
| | 2401 aactgctgaa actgaccatt ctgaaaggtc atcagcagga tattaacgag atctttaaac |
| | 2461 acttccgcaa agtgtacaaa atcaccctga atcagaaaga ttggcctggc aaaaacctga |
| | 2521 ttgatattgc acgtaatcag ctgcgtggtc gtctgctgtt ttgtaaagtt cattttcccg |
| | 2581 agaacgagaa gtatgtgagc attgagaaaa aaacctttcg tctggcaccg agcctgaaaa |
| | 2641 ccaaaattga aaatgttctg agcgtgatca aagatgacat tctgccgaac tttaccctga |
| | 2701 ataatgttgt gatggaaagc aacaacttcg acattgcagc aaaaaccaaa ggtaaaaaac |
| | 2761 gcctgctgaa agaagaatat agcaaaggcc atcgtgaaag cggtgaaacc cgtaaagaag |
| | 2821 cactgctgcg cgaaaccgat agccgttgta tttattgcgg taaaggtatt gatctgagca |
| | 2881 acgcacatga agatcatatc tttccgcgta aagccggtgg cattaacatt tttggtaatc |
| | 2941 tggttgcatg ttgcagcgtg tgcaatgaag aaaaacgtgg tcgcacaccg ctggaaagcg |
| | 3001 gtatcctgcc gaaaccggaa attgttagct ttattaccaa cgatctgaaa aagaaaatcc |
| | 3061 ttgaggatgc ccagtatatc aacaccctgg atttcaacaa atatatgagc catgccagca |
| | 3121 ttggttggcg tcatatgcgt gatcgcctgc gcgaactgac cggtaacaaa gagctgctga |
| | 3181 ttaaacgtca gagcggtatt tataccgcct attttcgtaa atggtggggc tttatcaaag |
| | 3241 aacggggtaa tcatggtcat catgcactgg atgcagttat tctggccagc aaaaaaagct |
| | 3301 atgcagaaga tggtaaagtc gatatgacca ttaaaccgtg tggtgaagat ggcaaagaat |
| | 3361 ttgatattga acgccacctg agcgaaatga aagaatttcg tcgtgataaa ggtggtaaaa |
| | 3421 gcgcaccgct gcatgatcgt aatccgctga gctttaaaaa cgatataatc acccgtcgtt |
| | 3481 ttatggtgac cgaaattgaa tgcggcaaag aagcagtgat tatctctgaa gagtatcgca |
| | 3541 aaaaactgac cgaagccttt aaacgttttg gtattgccaa aggcaaatat ctgaccgatg |
| | 3601 aacaggcaaa agatgccggt ttttatctgc gtaaaaatgg tgaaggtgtt atgtccctga |
| | 3661 aatgcgaagt taaaggcacc ggttataatc agatgatccg catcaaaaac aacatcttca |
| | 3721 aaaccaacgt gcataatgtt ggtgttgccg tttttctgga tgagaaaggc aaaaagcgtg |
| | 3781 catgtgagct gaaaaatccg cgtctgagca aacattttgt taaaccggca gaacaggtga |
| | 3841 aaggtaaggt gatttttatc ctgaaacgcg gtaatatggt taccgtggaa ggtgaagaaa |
| | 3901 tgatatatag ggttaagaaa cttggtactt ctcctgttat agaagccatt gtcggtagtg |
| | 3961 atggaaaaac aaggactgta tcagctacta aactcttgaa aataaaccat actaaaaagg |
| | 4021 tttaattaaa ttgtcctaac ccacaatgaa ggtgccattt ctggcgtaga ggtttaggct |
| | 4081 ggacaaaccc aaacgtacat accctcaccg tataggcgtt ttacaattaa ggctctttta |
| | 4141 attttcgcaa accattaagg atttatttca aaaaacaact tttctaaact tttatgaatt |
| | 4201 atcactgaca aaacctttga attaaaatga aaataattcc gttacagtcg ttgttttcaa |
| | 4261 agagccttaa ctgtaacccc atatatcagt tataaacact atattctgcc atgttacagt |
| | 4321 cgttattttc aaagagcctt aactgtaacc taccagtgag ccgattcata aggcgaacag |
| | 4381 taaggcgtta cagtcgttgt tttcaaagag ccttaactgt aacgagaagt catttaataa |
| | 4441 ggccactgtt aaaaagcttt ttacagtcgt tgttttcaaa gagccttaac tgaatttgca |
| | 4501 aacataatta aagtgtaata tgattacacc atattgccta aaaatcaata gtattataat |
| | 4561 agaataaact attatataat aagtcaatat aaatcctaaa ttgtatagta ttttcaaact |
| | 4621 gttatggggt tcaaagaatt atgagtttat taaaatatgg ctatttgggc atgttttttc |
| | 4681 ggggaaaggg ggtctcacgt acacgaaaag ctaatttttc agaaaagcgt aaaagatagc |
| | 4741 tttccccata actgagacaa aagagagatt gatatacttt tggatttgtg catataatgg |
| | 4801 caaaagtggg ggattcataa ctatgataca tgttgctcca tctgattgca gatagttaaa |
| | 4861 tgataacttc taatacatgc tatccatttg gtgggaaagt cgcaacagtc gaaacaccat |
| | 4921 catttaaatg catgattagt acagccaaag aaggcgtttt caagaaacag tccgtagttt |
| | 4981 ttcttgatat taatatgggc atggctaatc gccgacctgt cgctaagctg cattacaatg |
| | 5041 acatactcac taaagaacaa cgtttgcaat ttcacgatat catagtaagc gtgatagatg |
| | 5101 aatccggcat gaatggacta tctctctatg atacactcag ggacttgttt caaggacttc |
| | 5161 gcaaagaggg cattggttct gggtttttta cagataccca ttgatgacgg ctgagaagtg |
| | 5221 aaaagggaat aacttaaaac aattgtggag acatactttt caaaaaatcc ccttcccttt |
| | 5281 acctttgaaa tagtctgttg agataaagtg aaagaagaaa aaataaggga agacgttagg |
| | 5341 gattatttaa actcttttct cattgaaggt ggttattgtt ttaatgaccc taaaaaattg |
| | 5401 tgtttagcca ttaccagttt gtcgaacgac atatatatct tcagcaaaaa agatttcctt |
| | 5461 gtg (SEQIDNO: 53) |
| MHDT01000 | GCTTATTCAGGAACTTAACGGCAACACGACAGGCGATAGGCAAGAGGCTATAGGCAATGGGCGAAAGACAA |
| 042.1_ | AAGGCATCGGCGCATTGCAGGCAAAACTTAAAGAGGCGAGGGAATCCGAGTATAAGGCAAAAGAAGACGCC |
| organized | TTNNNNNNNNNNNNNNNNNNNNNNNNNNNNNNNNNNNNNNNNNNNNNNNNNNNNNNNNNNNNNNNNNNN |
| | NNNNNNNNNNNNNNNNNNNNNNNNNNNNNNNNNNNNNNNNNNNNNNNNNNNNNTTTAGACGCCCTTAAAA |
| | AGGAAAGGGAGAAGGGAATACTTACGGACATAGTGGACAGGGAAAAGAGCATCAGCGCCATAGAAGGAGA |
| | GGCGATAAAGGCAAAGAAGAAATACGAACTCGAAAGGCTTTGCTCTCCTGTTGACGGCACTGTTCACGGACT |
| | TGCCTCATACACTGTGGGAGGAGTGGTAACTCCTGCCCAACCCTTGGTCACGGTAGTGCCTGAAGGGACGCCT |
| | CTTATAATAGAAGCGACGGCATTAAATAAAGACATAGGATTTTTAAAAGTCGGGCAGGAGGCGGAGGTAAA |
| | GTTAGATACATTTCCGTTTCAGAAGTACGGCACCGTAAAAGGTAAGGTAACGGCCATAAGCCCGGATGCCTT |
| | CGAGGATGAAAAGTTAGGACCGGTGTATAAGATAAAGGTAACTATCGAAAAATTGAAGATCTATGCGGACG |
| | ACAGGTATATACCCGTAGCGCCCGGAATGAGCGTGTCGGTAGAAGTGAAGACAAATAAAAGGCGGATAATA |
| | GAGTTTTTCCTTTCGCCGATCGTCAAATACGCGAAGGAAAGCCTGACTTTGAGGTAAAAACGACAGACTTAG |
| | GGCGATAGGCGATAGGTAATGACGGATTTAAATTGGCAGCTTAGGACTAATGTCTAAAAGTGAGTCTGACCT |
| | TAAGCGGATGATAACTAAGGAGTATAAAATCGAAGATATAATAAAACCGAAAGGGAGGCCGAAGAAAGATG |
| | AAGACAAGAATAAATAGAACCGTCCCGTTTTTTGTATCGAGGAGTATTCTCTCCCCCAGGGAGAAAGAGATT |
| | TTGGAGCGCATGAGAGACGGCAAAACCGACCTCGAAATATCCGCCATTTATACCATCAGCGAACACACTGTA |
| | AAGTTTCATATAAAGCATATCCTGCGTAAACTGCAAGCTTCAAATAGGACACACGCCGTAGGCATCGCCATG |
| | CAGAAACGCTTGCTCGATTAATTGACATCGAATCTACCCCCTACTCAAATGAGTAGCTTACATTGGGGAAGGG |
| | GAATGTGGTATATAAAGGCCTGCATGTCTTGCAGCCGAACTTATAGGGAAGGTATGAACGAATTCAGTACGG |
| | CGTAAAAGAGTAAAAGATTGTGGAAATTACTGCTCAACAGAAATGCAAAAATTCCTGGAAGACTATTCCGAC |
| | AGATTGCCTCGACTGTCAATGTCCGCTCCGAGCCGATTGCAGAGGAGGATGCACAGCTGCATCAGATGAGGT |
| | ATATGGTTCACAATATCGGATTGATCCAATCTGTGAGTTATCTTGTTAAAAAAATTAGGTTGGTTTTTCAACG |
| | ATAAGGAAAGAGCATGCCTTATTGAGATCGAATAACGTGAGGCAAGATATAATCTTGCAGATTACTCTAAGT |
| | GGGAGGATTGTATGCCTGACATGAAAGATTTTTTAAATCCGAAATCGATGTTAACGCCCGGCATTGCAGGCG |
| | GATTAACAGCCAGTGTTTCTTTAACATTGGCATCTGCATTTGCAATAAGCTTCAAATGGTCGGCACTTGGCAT |
| | CAGTTTTTTGCTGGGGTTGCTTATAATGATTTCCATGCGGAACTCTATTCCCTTATTGCAGCGATTTATATATT |
| | GCATCCTAAATTCGCTGATTGTTTTTTCTATGGCGTTCGGCGCCGGTAAGGGCATTGATTCGCAGCCTCAGCTC |
| | GATCCTAAGATTATTGACAAAATTGTGAATACGCAATTATCAGTAGTTGAGCCTCAACCCCCTTCGGTTTTAG |
| | CGTTATCCAATGCATATGCGCAACAATATCCCATTAAATCAGATAAGCCTAAAAAAAGCACTGCTCCTTCCGG |
| | AACCGACAAAAAAGGGACAGGCAGCAATGAATCAAAGGAAAATAATCTGACCAAGAAAGAGATGGAACAG |
| | TTAAAAACGTATATTCAACAACAACAGAATTATGATAAGCGATGGTCGTGGTAAGAGAAAAGTGAAATTTTA |
| | AGAACAGAAAATGGGGAGGAGAAAATGTGTAAAGATAAGTCAGTGAGTCATTTAAACGAGTTAGGATGGAA |
| | TGTGGTAAGATTACCTAAAGAAAATATTAATCCGCTTTTAACTCTTTCCAAATCCAATAGCTATCTTGAAACTT |
| | TAGGAGAAATCTCTGATTTCGTGATAGAAGATCAACCCAAGCCCCCAGAAATTGTCCAAGACCAATCAGTAG |
| | CAGAAATTTCAGGTTTGGAGACTGACAAATTTGAATTGGGAATAGGACTAAAGTTTTTAGAAAAATTCCTATC |
| | TTTAGTGGGAGTAGCGGGATTAGGATTAGAAGCAAGCTTTAAGAATGCAGAATCCATTCAATTTGCCTATCA |
| | GAATGTACTGACAGATTTTGTCTATCCAGTGAAGATTGGAAAGTACTTATTGAGTGTCAGTCCTGATGTCAGT |
| | AGCCCCTTTATGGAGCATATTAATGAAGAGGGAGAGGCTTATATTATTACAGACACGCTTAGATCTAACACAT |
| | TCGGTATTGTTGCCTATGATGAAAAAGGTGTAAAAATTGATTTGGATATTTCTGCATTAAAGCAACTTCTTAG |
| | CGCTACTCCACATATTGAAGTATCTAAAGATGAGAAGAAAGTTGTTTCTTTTAAGGGGGATAAATTTCTGCGT |
| | TTTGCCTTTAAGGCAATCGGCGTATGGGTGGAGATACGGGACGGCAAAGCGAGATTTAAACTGAATAAGCCG |
| | GAAGGACCCATTGCACCAATGAAGGCTTTACCGTCCACTTTAATTAGTCCCAATGAACCAACACCTGTAATTT |
| | TCGGTATGAACACCCTAATCAGACTAAAATGAATCATAATAACTAAAAAGGAGATAAAACATGTCGAATCAA |
| | GAATCGATCAACAGAGGTCTTGACAGGGCATATAATGAGTTGAAACCTGAAGATACGATAAAATGCGATATA |
| | GAGTCTGTGCGTATCGTTATCTTTTCAGATCATCACAGGGGAGTTCGCGATGGAGCTGACGACTTCCAGCGGT |
| | GTGAAGGGGCATATTGTGCCGCACTCGGATATTATTTAGAGAGAGGTTATAACCTCATAGTTCTCGGCGATGC |
| | AGAAGAATTGTGGGAGTGTAGGCCCGCCCCTGTAATTAGAGCTTATGAAAACACATTGAAGCTTGAGGCAGA |
| | ATTTTATAAGAATAGGGAAGACCGCTACTGCCGTATTTATGGAAATCACGATGATTACTGGAGGGATACGGG |
| | TAGTATTAATATAGGTGGGGAACAAATAACGGCAAGGGATAAGCTGATGATAAGCCTGCATGATAAAAACA |
| | ATAAATCTATTGGGGACATTTTTTTTGTTCATGGTCATCAAGGGACTTTTGAAAGCGACCGCTTTGGGAAGAT |
| | CAGCAGGTTTTTTGTAAGAAACCTCTGGAGACCCTTTCAGAGGATTACTCATATAAAGTTGACTACACCTGCC |
| | AAGGATGCAGAATTGCGCGGCGAACACGATACGGCAATGTATAATTGGGCTTTAAATAAGAATAATGGGCAA |
| | AAAAAGCTTTTGCTGATTGCAGGCCATACGCATTTGCCGGTATTCGCATCAAAAGATCATATAGGAAAGATTA |
| | GCGATGAGCTTAAATCTCTTCAGCAGCAGCTTGATAATGCGAGGACATCCGGCGATCCCCATGTTGACGGCAT |
| | TGCAGAACAGACAGCGGAAAAACGGGCTGAGCTTGAATATCGAAAGGCGCAAAAGAATATGAAAGAGGATC |
| | AGAAAGGCATTTTTATGGAGAAGCCATGCTATTTTAATTCCGGCTGTTGTTCATTCAGCGATGGAGATATAAC |
| | AGGATTGGAGATTGCTGACGGAAAAATAAAGCTTGTTAAGTGGCCCAAAGATAATCCGAGAGTAGAAGTTCT |
| | TGAGGATGCCGACTTGAGAAGCGATATTTTTGGAAGCTTATAGCATTGCTGTACCTTAAGTTTTTTAAGATGA |
| | CGTTTGTGAAAGGAGACGGCAATGATTTACGAATACGAGATAGACGGCTTGACGGTGCGGACAGGCGATATC |
| | ATCTGCACTTCTGACGGCGCTGCGGATACCGATATCAAAGGCCAGTTCTGGCGGCTGCTGGGGAAAATAATA |
| | CCCGGCGAGGTCGATCACATTATTATTTATGTAGGACCAAACGGTCGTTGCGTAGAAGCGGGAGCAAAAGGC |
| | AGGGTGATAACCTTTGAGATTATGGATACTACATGGGATTTTCAGAAGATGATTGCAAAAAGGGGAATAATA |
| | GATACCCTGTATGGAGCAGCATATCCGCTTCAGGGGAAGAGTCTGAGCGAAACGGAGATAACAGGAATACG |
| | GGAAGCGGTTGCCGCGTATTGCCTGAGGCAGGCTGAATTGGAGAAGCCGTATAATATGAATTTCCTTGATTCA |
| | ACCACCGAAGATGCATACTACTGCAGCCAGCTCGCTTACCTTGCATATTTCAAACACGGCATCGACCTGAATA |
| | CCGGCAAGGGAATACCCGAAATACCAGGGACTGAGCGCATAATCTTCCCGCAGGAGATATGGAGCGGGTGC |
| | GGGCATAAAAAAGCGCGGTAAACAAGTTTCGCTCTTGCCAAAATCCATGCTTATTTTGTAGAATAAGAGTCTA |
| | AATTTTTATTTTCAGGAGGGTTTGCGTTGAAAGAGGGAATACATCCGAAATATAAAGAGGTAAAGGTCGCCT |
| | GCGCCTGCGGAGAGACTTTTACTACAAAGTCCACAATGGACAATATAAAGCTCGATATCTGCTCCAAGTGCC |
| | ACCCGTTCTTCACCGGCAAGCAGAAGATATTGGATGCCGAGGGAAGGGTAGAAAAGTTCAAAAAGAAATAC |
| | GCTAAGAAGTAGCGAATAAACATATCGGGGTTAGAGCGCGGGGTCGTTTGCGCTCTAACCTTTTTTTTATTAA |
| | GGAGATATATGCATAAGATAGGCGGACAGGCCGTAATAGAAGGCGTTATGATGAAATCTCCCGAAGGCTGG |
| | AGCGTGGCCGTAAGGGGCCCGAAGGGCGACATCAGCTTTAAAACCGTAGTAACGAAAAAACCGAACAGGTT |
| | TTTCAGGCTGCCCTTTGTAAGAGGCGTGGCTGCCCTTATTCAGGCGCTTTCCATCGGCGTTAAGGCTATCGAA |
| | TTTTCGGGAAATATCGCGTATCAGGAAGACGAGAAACCTATAGGCACGCTCGGCATGGGACTGACCATCGGT |
| | TTCGCGATTATTTTAGCCATAGCCCTTTTCAAGTTCCTTCCGCTTTTTCTCACGACTTTAATCGGAGGCGTTTCT |
| | GAAGGCGTTTCAAAAAGCTCCTTTATGTTCAACTTTATCGACGGGACGATTAGGGTGGGAATATTTCTTTTCT |
| | ACATATTCGCCATCGGGCTCTGGAAAGAGATGAGGAGGATATATCAGTATCACGGTGCGGAGCATAAGGTTA |
| | TTTACGCTTATGAAGCCGGGGAGGAACTCACGGTCGAAAACGCGAAGAAATATAAGCCGTATCATCCGAGGT |
| | GCGGAACCAGCTTTTTGCTGATAGTGATGGTTATCAGCATGCTCGTATTCATGTCGATCCCGCAGGGCTGGTC |
| | TTTTTCCGGAAAGCTGATGTCAAGGATTGTCCTGTTGCCTGTGATAGCGGGCATTTCTTACGAGACGCTGAGG |
| | CTTTCGGCGAGAATGAAGGATAATCCGGTCGTAGGGCTGATCGTCCTTCCCGGACTTTTGCTCCAGAGGATGA |
| | CCGTGCGGGAGCCGGACGACACGCAAATAGAGGTGGCGCTGGCGGCAATGAACGAAGTCCTGAAATTAGGC |
| | GCGGATAAAGAGGTTTGCAAACAGTGTTAAAAAAGCTGCTTACGATAGAAGAAAAATACGAAGAGATAACC |
| | AATTCGCTCGTAAATCCGGATGTGTTATCGAATCCCCAATTGTATCAGAAATACTCGAAGGAGCAGTCGGATC |
| | TTTTTCCCATTGTCGAGAAGATAAAGGAGTATAAGAAGCTTCTTGCCGACCTCGAAGGTGCGGAGGAACTGC |
| | TTAAGGCCGGAGACGGCGATTTAAGGGATCTTGCTCAGGAAGAGATGGAGGAGCTTAAAAAGAGGAAGCCC |
| | GTTGTGGAAGACGAATTGAAAATAATGCTCCTCCCCAAAGATCCGAGGGATGGAAAGAACGTAATCCTCGAA |
| | ATACGGGCGGGAACAGGGGGCGAAGAGGCCGCCTTGTTCGGCGCGGCCCTTTTCAGGATGTATTCGAAATAC |
| | TCCGAATCTAAAAGATGGAAAATAGATATTATAGATTCAAGCCCGACGGGACTCGGAGGCTTAAAGGAAGTT |
| | ACGGCTAATATTACGGGCAAAGGCGCTTACAGCAGGCTTAAATACGAAAGCGGCGTTCACAGGGTGCAGCGG |
| | GTTCCCACGACCGAGGCGTCCGGAAGGATACATACCTCGGCGGCAACTGTAGCCGTGCTGCCCGAGGCCGAA |
| | GAAGTGGACATTAAAATAGAGGAGAAGGATTTGAGGATAGATACCTTCTGCTCTTCGGGCGCAGGCGGGCAG |
| | AGCGTTAATACGACATATTCCGCCGTAAGGATAACTCATATCCCTACAGGGCTTGTCGTTCAGTGTCAGGATG |
| | AGCGCTCCCAGTTGAAAAACCGCGAGAAGGCCATGAAGGTGTTACGTTCGAGGCTTCTGGAACTCGAAATAG |
| | AAAAGAAGGAAAAAGAGCGTGCCGCCGACAGGAAGACGCAGGTCGGAAGCGGGGACAGGAGCGAGAGGAT |
| | AAGGACTTATAATTATCCGCAGAACCGTATATCCGACCACAGGATAGGGCTTACGCTTCATAAGCTCGAACA |
| | GGCTCTTGAAGGAAATCTCGATGAGCTTATCGACGCCTTGATAACCCATTATCAGGCGGAGAGGCTTAAGGA |
| | ATTGTAAAAATCGTCAATACGGGATCGAATTCTGCTATAATATTTACAAAACAGAAAGGGGCGGTACGCACA |
| | TAGACAGCAGGACTAAGGCATTAGAGGCGGCGCAGGCAGCGCTCGATAAAAAGGCAAAAGAGACGGTAATC |
| | CTCGAACTGAAAGACCTTACGATAATCGCGGATTATTTCGTTATCTGTTCCGGCGAAAGCACTACGCAGGTCA |
| | AGGCAATCGTCGAAAACATAGAGAAGATATTAAGAGAGCACAAGCAAAGGCCATTAGGGATCGAGGGGCTG |
| | AGGTCCGCCGGATGGGTCTTGATGGATTACGGGGATGTGATAGTCCACGTGTTCGAAGAAGATACGAGGGCG |
| | TATTACGAGCTTGAGAAATTCTGGCTGGACGCCCCGAAAATAGCGGTAGAAGACAAGTCCGCTAAGAAGGCC |
| | GCGGGAAGCAAAAAGCAGTATGTATGAAGGCGCGGATATTATGGGTCGGAAGAACGAAGGAAAAATATTTG |
| | ACGGAAGGGATTAACCGTTACCTGAAAATGTTAAAGCATATGGCGAATGTTTCCGTCATAGAGGTGAAGGAA |
| | GAGAAGGGCAAGACGAAGGAGAACGCCCTTTTTGCGGAGGGAGAGCGTATATTGCGCCAAACAGCGTCTTAT |
| | ATCCTGCTCGATGAGACCGGGAAGGAATTCAGTTCTAAGGATTTCGCGAAGCTTCTCGGCGAAAGGGATCAG |
| | GTCGATTTTGTGATGGGAGGCGCGTACGGGGTTTCCTCTGAAGTTAAGGCAAAGGCAACGGGTAAAATAGCG |
| | CTCTCGAAAATGACCTTTACCCATGAGATGGCAAGGCTGATATTTTTGGAGCAGTTTTACCGCGCGATGACAA |
| | TCGTCAAAGGCAAGGAGTATCATCACTGATGGGCAAGATAGCGATACTTGTATTTTTGTTGTTCCTCGGAGTC |
| | CTCGGTTTTTTTGCGGTCGAGAACAAAGACGCCGTGATCATGAAAATTCCCTTCGGCGACGCTTATGAGATCC |
| | CGAAGATAGCGCTTGTTCTCCTTTCCTCCACCATAGGCGCGCTTGCCGTCCTTATCGTGTTTTTTATAAGAGAT |
| | ACGAAACGCGTAATCGAAAATCTCCAGTATCAGAGGAGGCAGAAGAAAGAGGCCAGGATTCAGGAATTTTA |
| | TTCCAAGGCGTTGAACGCCATAATGGGCGATAAGGAGGAGGAGGCGCGGGAAGCCCTCGGCGATATATTGA |
| | AGGAGGACCCGGAATATATAGACGCCCTTTTACGGCTCGGCGACATCTCGCTCAATAACGGAAAATATGAAA |
| | CCGCGTTCGATTATTACAAAAAGGTGAAGGACATAAACCCGAAGAATCTACAGGTGCTTTTTTCCATGGAAA |
| | CAGTGATGGAAAAGCTGCAGAGATACAACGACGCGTTTAATTACCTCGAAGAGATACTCGATCTCGATCCCG |
| | AAAATCTTACCGGGCTCTACAAGAAGCGCTCTCTCCTCGAAAGGAAAGAGATGTGGGACGACCTCCTTTCCA |
| | CGCAGAAGGCGATAATCAAGCTCGAACATAACGAGCACGACAGGCAGCGGGAAGAGCGCAGGATGCTCGGT |
| | TATAAATATGAATACGCCCGCGCGAGCCTTGAAAACGGAGATGTTGAAAAGGCGGAAAAGGCGTTCAGGAC |
| | AATGCTCAAGATGGACGGTAATTTTGTCCCGGCGCACCTCGGTCTTGCGGAAGTCATACTTATGAAAGGAGA |
| | GACCGAGGAGGCGATAAACTTTCTCGAAAAGGCGTACGAGCAGCTCAGGTCGATAATAATAATCGCTCGCCT |
| | TGAAGACCTTCTCATCAACGTAGGAGAACCGGGCAGGCTTATAAGATTCTATAAAAACGCCATAGCTAAAAA |
| | TCCTCAGGACAACGGGCTGAGATTTCTCCTCGGCAAGCTCTACTACAGGCTTGAAATGGTCGATGACGCCATG |
| | GAGATGCTGAATTCAATCGATACCACGGTCTTTTCGGTGCCCGAGCTGTATAGTTTGAAGGGAGAGTTATATA |
| | TAAAGAGGAGCCAGATACAGAAGGCGCTGGACGAACTTAAAAAGGCATGCGGCATAAAAAGGCCGTTTAAG |
| | ATCCAATACTGCTGCTCAAACTGCGGT (SEQ ID NO: 54) |
| | GGATGATGTTGCAGACATCCTTAAAGGACGCAGGGAACAGAGAGGTATCAGAAGAACTCAGCAGACAAGGA |
| | AGAAAAGACTGCGGGAGCTTAAAAAATATCTAAATAGTATAGGATATGATAATTCTACCGAACTATTTAAAT |
| | CTATTTATAGCCTCGCCCATAAGAGGGGCTATGACTATGCCGATATGCCAACACCGGAAGAAATAGAGGAAA |
| | TGGATGAGAAGGAAAAAAAAGAATGGAAAGATACGCAGAGAAATTCAAGGTACAGGAAAGAGGTTTTGGCA |
| | GATGTCCGCAAGGTAATGTCCAATGGTGGAGCTTCGGATGAACAGATAAAGAGGGTAGAAAGTATTTTCAAT |
| | AAGCAGTATCGGCCTAAGAGATTTAATAACAGGATTTTGACCAAGTGCAAGGTCTGCGGAAAAAATACGCCA |
| | TTAAGAAGAAATGTGAGGGAGTTGCTTCTTGAAAATATAGTTCGCTTCCTGCCGTTAGAAAGCGAACTAAAA |
| | GAAACTCTTAAGCGAACAATTCTTGAAGGACAGCAAGGAAATATCAATAAACTATTCAGGAAATTGAAATTT |
| | AACCAGAAAGACTGGCCGGGCAAAAATCTTACGGACATAGCAAAGAACAAACTGCCGGGCAGACTGCCTTTT |
| | TGCAAGGAACATTTTGCAGAGAATGAGAAGTTTACCACTATAGAGAAATCAACTTTCAGGCTTGCGCCTTCTT |
| | TAAAGACAAAGATAGAAAATGTCCTGACTGTGATTAAAGACGAAGTAATGCCTAACTTTGCTCTCGATAGGG |
| | TAGTAATGGAATCTAACAATTTCGATATAGCTGCAAAAACGAAGGGCAAAAAGAGGTTGGCAAAGGAGGAA |
| | TACAGCAAGGGGCATAAGGAAAATAGAGAAACTCTTATGGAATCCCTTTTAAGGGAGACTGATGGAAGATGT |
| | GTTTATTGCGGTAAGACTATAACTTTAGCCGATGCAAATAAAGACCATATATATCCTAAGAAAGCAGGAGGC |
| | AGCAATATCTTTGCTAACCTTGTGGCTTGCTGCAGGAGTTGCAATGAAAATAAAGGGGGGCGGACTCCATCG |
| | GAAAGCGGCATTATGCCTAATCCAGAGGTCGTTGCAACGATTAAAAACGATTTAAAGAAAAAAATCCTCGAT |
| | GATGCAAGAAGCATAAAGCAGTTAGATTTCAACAAATACATGTCTCATGCCTCGATTGGCTGGAGGCATATG |
| | AGGGACAGGCTGAAAGAGCTAACCGGAAACGATAAATTGCCTGTAGAGCGGCTGAGCGGTATTGTTACAGC |
| | ATACTTTAGAAGGTGGTGGGGATTTAAGAAGGAAAGGGCTAACGATAAGCATCACGCGCTTGATGCTGTTAT |
| | TCTTGCTTCAAGGAAAGATTATACGGATGAGGGGCTGGTGGCTATGACGCTTAAGCCTGCAAATTCGGATGG |
| | ACGGGAGTTCGACCCTGAAAAGCATATCAAAGAATCGGAAGAGTTTAAGAGAAACAAGGGCAGCAGAGGGT |
| | CTGCCCTCTACGACAAAAACCCGTTGTCCATTAAGAATGGCAAAATTGCTCGAAGATATATGGTTACGGAGA |
| | TTGAAAGAGGGAAGGAAGATGCTGTTATTTCTGAGGAATGGCGTGAAAAGCTGAAAGAAGCATTTGACCGCT |
| | TCGGGGTCAGCAATGGAAAATGTTTAACTGATTTACAGACTAAGGAAGTTGGGTTATATGGTCAGAAAAATC |
| | CTATGAGCCTTAAATGCGCTGTCAGGGGCGCAGGCAAAGGGCAGATTGTGCTGATAGGCAACAATGCATTTA |
| | AGACGAATGTTCATAATGTCGGCGTGGCGGTTTATCTCGACGAAAAGGGCAAGAAGCGGGCATGCGAACTTA |
| | AAAACCAGAGACTGGCAAAACACTTTGTAGAGCCACAGGATGAGATTAAAGGAAAGATATTATTTACCTTGA |
| | GAAAAGGGGACACAGTTAAGGCGGAAGATGGAAATATTTACAGG (SEQ ID NO: 55) |
| | AAACGAAGCGGAGAGCATTCCCTTATTGATCGACAAAGATGTTGACGACGAGATATTTATCGATGAAGGGAT |
| | TTCTCTGATAAAAAGCGGCGATTCATCGCAGATAGTGTTATCGGGGTTCGGCATTTTTTTAAGCAAAAAGAGC |
| | GAGCGTCTTATTGTAAAGAAAAACACTAAAGTTGTTTATGAGTTTCCCTTTTTCAGGCTGAACGAAGTCATCA |
| | TGGCTTCCCGCGGCATATCGCTGTCGTCGGATCTTATAGAGGAATTGTGCGAGAGGGGTATAAGACTGAATTT |
| | TCTGAACAACAACGGCAAGCCGTACGCAATGCTTTCTTCTCCGATGCTCTCCGCAACCGTCATATCAAGAAGA |
| | GAGCAGATTATTGCCTTTACCGATACGCGCGGTCTTCAATTCTCAAAAGCTATTGTGGAGGGCAAAATCAGAA |
| | ATCAAGAAAGACTTTTGAGATATTTCGGCAAATATATTAAAACTGTTGACAGCGCCAGGTTTGACAGAATAG |
| | AGGAGATAACAGATGTCATTAGGAAGATGAGCAGGCAGGTGGAAGGTATTCACGGGCAGAGCATTGAAGAT |
| | GCAAGAGGCAGACTGATGGCAATTGAAGGCGCTTCAGGTAGGCTATACTGGGACGGCGTTAAGGAGATAATC |
| | GGAAATAGAATAGAGTTTTTCGGGCGGGAAACGCGGGGAGCCATTGATGCCGTAAATTCCCTGCTGAATTAC |
| | GGCTATGGGATTTTATATTCCCACGTCTGGGGCGCCGTTATAAACGCCGGGCTTGAGCCGTTTGCGGGGTTTC |
| | TTCATGTGGACAGGCCCGGAAAGCCGTCTCTTGTTTTGGATTTGGTCGAAGAGTTTCGTCAACCTATTATCGA |
| | TAGGATTGTAATCGCCCATATTAATCTTGGAGAATCAATCGAAATTAGAAATGGCCTGCTTGAAGCTGAGACC |
| | AGAAAGCTGATAGGGAACAAAATTATTGAAAGGCTGGAGAGTCAGGAAACACTTGACGGCAAAAAATATAA |
| | A (SEQ ID NO: 56) |
| | GTAATGAGGATTCCTGTAAAATGCCGTGTCCGTGATCATGAACGCCGAATAGCCGAACTCCCAGAAGCCCCA |
| | GTGGTCCGAGAAATTAACGCCGGGGATCAGCGAAATCCCGTTGAACGATTCAGCGGGCAGTGAAGATGACTC |
| | TTGAAAATATCTCTTCACTTTTTTCGTAAAAGACATGGAGCGGATATTTCCGACAAACGTAATAAAATTGCCC |
| | TTGTCAGGATAAAACCATTTGAATATCGGAAAAGGATAATACTGAGAGTTTTTTCTGTCGGAGTAATATCCGA |
| | GCATTTCGAGGGAGATCATGCCGCAGACTTTTATGCCTTCCTGTTTAAGGCTTTTAGCATAAACGTAGCTTCCC |
| | ATATGTTTTGTCATAAAAGTGGGAGGCTCTTCAAGACTGAAGGCAACAAAGCGGACCGTCCTTTGGAAAGAT |
| | TTTGTTACGGCAAGCCGCGCCAGTTCGAGCAGTCCCGCAATGCCGCTTGCATTATCGTCCGCGCCCGGAGTGC |
| | CCGTTACGGTGTCGTAATGCGCGCCTATTACGAGAATCCCGTCTTGCGGGTTTCCGGCGCCTTTTATTTCGCAG |
| | GATACGTTGTAATATGTATTGCCGAGATACCCAAACGGCTGCTTTCTAACCTCACAGCCGTAAGAGCGAAATT |
| | TTTTTTCTATGTAATCGGCGGCCTTGTTGAGCTTATCTGTATCGCGATAACTCCTCTCGCCTATATCGTGGGCG |
| | AGGCGCTTGACGGTTTCGATAAGATTAGCTTTTATATTTTCCATTTTGCCGATTAATGTTCATAGCTTTATAAA |
| | TTCTTTCGGCGCTAAATTCGTCCTCATTGTCCATACCGATGCAGTGCCGTTTTTCAAGCGATACACAACCAGCG |
| | GGTCATAGCCGTATTGCTCCACAAAGGGTTTCAGAAATTCGCGCTTTTGCACTATCTCCTTCTTTAGTTCAATA |
| | TCTTCCGTAAGTTCAACGACCCCGCTGACTCTTATTTGTATAAGGTTCTCAAAGCTGCCGTTGTTAAAGCTCAG |
| | CTCGACCAGCGGGTTTTCGGTCAATTGTTTATGCAAGTCTTTCACCTTTCCCGTGTGAAACAAAATACCGTTCT |
| | CATCAGCCCTATACATCAGCATACCGCGAACGCGGGGCTTATCACCTTCAATCGTTGCCATGTGAAATACCGG |
| | GTTTGCATTAAGAAACTCCAAAATTTCAGTCTTGTTCATATAAGCTTCCTCCTAACGATAAATGTTTTCAAGCC |
| | TGCGGATAATATCGAGTTTTTCTTTCTCTGTTGTTCTCACTCAGGGTTACTCCATTTGCGGCCCTTTGCCTATCT |
| | TCATTTTGCCCCTGAAATATCTTTGTTTGACTTCCGCGGTCTTTTCCTTCCATTTGTCCACGCCGTGCTGTTTAA |
| | TGTAGCACAAAGCAAATAGTTTAATGTTGTGCGGCAGGTCGCCGGCCTTGTCCGTTGCGGGATTTAATTTGGC |
| | GCAGGGGAATTCCGCGCACTCGAAGCAGAAGTCATGTCCGCGTTTGCTGATGCAGGCATAGGTCTCGCATGT |
| | GCCCTCAACAAACTGGCAGCGTCCCTTGAGCGGCCTGCATCCCTGACATGGGATGCCGTTCCAGTTAAATTTT |
| | TTGGTCAGCATTTCTCTCCACGAAGGATCGTCCTTTACTTTGTACGCTGGGCAGTCACCGCAGTATCCCCCGCA |
| | CGGCGCAACTAATGTGATGTCATCTTTTGTTTCCATTCGATAAGCCTCCTGTAGCCGGTCTGTATACCTTTGCC |
| | TGGTTGTCGCGATATAGTTGATCGAAAAGTTCAGTAAATTGGGATTGTCCGGCCATCATATCCTCTTCTTAGA |
| | TTACACAGCACTAAGCTGTTTTTTCAATTCAGGAGTGTGAGTTAACATCGGGACAGCGACCAGGAAAGGCCG |
| | CTGTCCCGTTGCGGTCATACTAACAACAGCCCTTGTCTGATTTTGTCCCGCAGCAGGACTGGAACATGTTCTTC |
| | ATATTTCCATCCGTGCAGCACTTCTCAAAAATGGTTTTAATTTTTTCTTTTACGTCTTGACCGGTGATCTCAAG |
| | ACGATACCCATTCTCGAGTTCCGTAACGTTAGCGCTACAGCATTGTTTCTCCATTATTATTCACCTCCTTCCAG |
| | TCTTTTATTATTCGAGAACACTCGAACAAGACCGCCAAAAAAAACTATTCCATGAACTTTTTTATGGAATCGA |
| | TCACGTCCTTATTTACTCTGCAACGATAGATCTGCCCCTCGCGCTCACATGTTATAAGCCCGGCATTTTGGAGT |
| | TCCTTGAAATGATGCGATATTGTTGACCGTGAAAGATTCATGCAATCACAGATCTTTGTAAAGGCCTTTTCCA |
| | TGCTTCCATCAGACGCGCAACATGTTGCCCCTTTAATGCGAAATTCCGCCCCGGCCTTGGACCCCTCGATTCC |
| | GCAACAGTTTTTGTAGAGCATAATGAATATCTTCAATCGCTGCTCATTTGAAAGGGCTTTAAATATTTTTGTGT |
| | AGTTAGATAGTTTCACAACATTAGAATAAACTTATCGTGCGTTTTTGTCAAGCATTTTGACGAGAGGTAAAAG |
| | CTTTGGCGTTATTATCAAGAGTATGAAGGAGTAGGGGGATAAAATCGGCTGAAATTTTTACGAATGATATAC |
| | CGAAGCCTTATGAAATTCTTCAATCGCCATATCCTTATCGTATGAGAAGCCGGCAGGAAAAATGCCCTTCTTT |
| | TTGCAATACACAACCATCTCATCCAGCACAGCCATGAGTTCATCTTTCAGGTTGTTCACTTCCCCTAAGCCGTC |
| | ACAACCCCTCTGCCTTTCTTTTATAAAACTATATATCTGAACGTAGTCCTTAGCTTGTGCGTAGAGTCCTGAAA |
| | GATGAGAATCGGATTCCGCCATTTTAGTAATTGCATCAATCATGCATACACCTCCTTTCGACTGTATAGACGA |
| | AGGAAAGTGGGAAAAGGATACACCCTTATTTTTTCTTGTGGAATTTTAAGGTGGGTTCTTTGCGGTCGCAGGC |
| | GAATTCGTTCCGCTCGTCGCGGTAGAAACTGCAGTGCGCTTCCAGCGTTTTTTTCAGCCTTGCCCGCGCACGG |
| | TGGAGCCTGATCTTGACGGTATCAAGGCTTACCCCGAGAATCTCTGCTATTTCTCCGTTTTTGAACCCTTCAAG |
| | CTCGCTCAGGACAAGTATGGTCCGGGAATGAGGGGGAAGGGCATCTACATACCCCCGGATACATTCGCTCAT |
| | TTCCTTGCGGATCAATTGCACATCGGGGGGAGGAGTTTTTTTATCATAAAAAACATCTGTTTCTTCATTATCCA |
| | TACCGAGCGGCTGTTCTTGTCTGAACGACGGACTGCGCATCCTGTCGACTGCAGCGTTCGTTGCAATTCGGTA |
| | TACCCATGTCGACAGCCGGGAATTTTTTCTGAAATCCCTGAGTCCACGATGAACCTTTACGAAAACTTCCTGG |
| | GCTGCATCCTCGGCCTCGCCGGCCCCGATCAACCGCTGCAGGTAACGGAGAACAGGCGTCCTGAATTCATTAT |
| | AAATTTGTTCAAATGGCGTATCGTTGGTCATCGATTGTTGATCTGCTTTTTAATCTCCTGCATAGGAGTCATTC |
| | CTTCCCCAAAATTAAATCAACCATGATTTTACGAGGTTGGCAGAAAAGTTTTTTTACTCTTTCCCTATATTCTT |
| | CGGGTGTAACGTGTTCCTGATGAGTTCCTATCACGATTTTAATATTCGGGAATGCCTCTTCCAAAGCCGATTTG |
| | TATGTCTCTTTAAACGGGCATAGCGCCTTCATGCAGTAAGTAAAGTGTATCGCATCCACCCGGAATTCAGTCA |
| | GTGCCCTTATACGTTGCAATAATTTATCGGCGCCGCTAAGGGTAGGGCAGCCCGGACAATTAATAATCCCTAC |
| | CAAATCTAACGGGTCATCTTTTGGATAGTCCGCAAATGTTCCTCTTCTTTTTCTGAGATCGGCAAGACAGCTTG |
| | CCGACGAACAGCCCAAGTCCTGCGTCGCATTGCTGCATGTCAATATTCCGATTCGCGCCATATAATCACCTTC |
| | TCGTTTACAGTTTCTCTAAAAACTCTTTCCTCACAGGCGACCACGCGTCAACGGCCTTGCAAGGCATGTTGCC |
| | TGTAAAGGCCGAATGAACGGCGTTTGACGGTATCGCGATTACATCCCCTGCCTTCAAGGTAGTTGTTTCTCCT |
| | TCATAGGTAAAGGTCAATTCCCCTTCCAGTATCAGGGTGATCTGTTCCGCCTCGTGCGAATGCTCAGGAAATT |
| | TTGTGTCCGGCTCCATCTCAAAATATGTAAGCATCGCCTTATCGAGAGCTATTGCCCACATCGAGGCTCCCGG |
| | GACATTGCTTCTGAGCTTTAATTCTCCTTTGCGATAAATATGTATCTCGCCCATTGCTTCTCCCCTTTTTAGCGA |
| | TTTCATTTTTTTAGATATTTCATCTGAATATTCCAGAGTATAAGCCCCGCGCCTTCGGGTCTCGAAGGCAGAAA |
| | TTTTCTAATTTCAGAGGCGATAAATTCGGCATTGTCGGATTTGGCCGCAAGCTCTTTGGCCTTTTTATCAAAGG |
| | CAATAAGATAGTCTTTCATGTCTTCTATATCTTTTTTTGTTGAGACCGGCCCGTGCCCCGGAATTATTTTCTCG |
| | ACGTCCATCGTCATGATATAGTCGAGCGTTTTTAACCATCCCTCTATATCGCCTTCTCCCATGAACGGGTGATA |
| | ACCGGTAAATAGAATATCCCCCGCGAATAGAATTTTTTTGTCCGGCAGATAGACCATAATGCTTCCATCGGTA |
| | TGCGACTGCCTGAAATAGATGAGTTCTATTTTCCGGTCGCCGAGGTCTATTTCCATTCTTTCGTTAAACGTCAA |
| | AACAGGGTATGCGATCTTCGTCCCTTTCACATCCTTTTCGGTAAGTCCGTAGGTATTTGAGTTTTTCAGGGCTG |
| | TTTCGCCGCGAATTTTCAGATTATTACCGTCATTAGTGTGCGAGATGATCAGCGCTCCGAGTTTTGCGAATTCC |
| | GAGTTGCCGAACGTGTGGTCGAGGTGATAATGGGTGTTGAGGACATATTTAACGGGTTTCTTCGAGACTGACC |
| | TTATGTCCCTGATAAACCTTTTCGCCTCTTTTGCGGATATGAGGGTATCTACTACCACAATGCCGTCTTTACCG |
| | ATAATGATGCCCGCATTAGCGCCGTAGCTGTTCTGCGGAGTGCTGTTTTTAGTATCCACATACGAATAAACGT |
| | CGTCGGCTATCTTTGTCAGTTTCTCCGCGGCAATTGCAATTTGACCAAATGCTAAAGAAACGATGAATACAAA |
| | TACCCAAATCCTGAAAATCATCAACCTTTTCATAACATCCTCCTCGTTTTTTATTTTAATCCTTTACCGAATCCT |
| | TCAACAGTTTCCTGAAAACAACCATATTCGATTTTGTGAAGCCGTGCTTCCGATAAAATTCATGCGCCTGCTC |
| | GTTATCATTATCGGTAAGCAGGGTGATCCTTCCGCATCCGTTATTAATCGCAAATTCTACCGCATGCTTAATTA |
| | AACTTGTACCCACGCCTTTCCCCCTTGAATCGGGCGATACAACCATATCTTCAAGAAGGGCGACTTTCTTGCC |
| | GAGGGCGGTACTTATCGTGAATAATACGACTACCATTCCCTGAATGACGCCGTTAGATTCGCATACAAATACA |
| | GAGCCGGTTTCAGGGTTCTTAACGATCATCTCCAATCCCCTCCTTTGAGCCGTCGGATTGGGTTTAAACTCATG |
| | CTCTTGACCGAAAAGAAGGCCGAGAAGCTCAATGCAAGAGCCGAGATCGTCTGTATTTGCAGGTCTAACGGT |
| | TTTCATTAATATGCTTTCTCTCGTCCGATCACTTCATTCTCAAGAATTCTTCAATCACCGGACACAATTCGCTC |
| | CCTTTTTCTTCCTGCACAAAATGTCCTACATCATCATATTTTATTGTTTTGGAATCGGAGAAAAGGCTTATCCA |
| | TCTGCTCAGTTCCTGCTCTCTGAAAGCAATGTCTTTCATTCCCCATAAAATGAGAGCCGGCTTATCTTTTATTT |
| | TATCACTCTGCGACCAGAGAAAATCCAGCCAATCACTGGAATCAATAATCCGCTTCGGAAATGTCCAGCAAC |
| | CCTTCCGTTGCTCCGGTATTTCAAGCGCCTTGAAATAATGCTGATGGATTGAAGTGGGGAGTTTGGAGGTATC |
| | ACCCATCGCCTTTTTCATTATGACCCGCACAAAAAAATTAAAGCGTTTGATCAAGAACCTGCCGATTGGTCCG |
| | CCCACAAATTTGCTGAACCTCTCATAATAAGGATCTCCCTTGACAGACCACATCCAGGTATTCATGATGATGA |
| | TACGTTTCATATTTTCCGGAGAGTTGACGGCATATGACAAGCCGATAGGTCCGCCCCAGTCCTGAACAACGAG |
| | CGTTATTCCTTTCAAATCTAATTTTTCTATCAATAGTTTCAGATTTTTCGCATGATCTTCCGGATAATACGACC |
| | AATTCCACGGCTTATCCGATAAACCGAACCCGATAAGGTCAACAGCTATGCAGCGATAATTTTTCGAGAGAC |
| | ATTTAATCAGATGCCTGTATAGAAAAGACCATGCGGGATTGCCGTGAACCATAACTATCGGCTCTCCGCTGCC |
| | CTCATCAACATAATGCATTTTCCCCATTTCAAGTTCAAGAAAATGAGACTTAAACGGATACTCTTTTCTGTCTA |
| | TCCACTTATTCATGAAACCTCCTTATCTCTCATCTCCTTTTTCGCTCCGTGTTCATAGCCTGACCGATTCGCGCC |
| | GTTATAAACCGGTATTTGCCAAGTCGGTCCATGGAGAGAGAACTACCGCCAAACATTTCGATTATATTTCCAC |
| | AACCCATTCTTTTATCCAGTTGCTCTCAAGTAAATCCTGAATAACAAAAGGGTCGTTTATTATAATCCTATTTG |
| | CATCGTCAACGCTTTCGGCCTCAAATGTAATCATTCCACCCGTCCTGTCTGCAAAAGGCCCTCCGAGATACTT |
| | GCTCAATTTAAGCTTGTGCCAATACTCAATATGCAACGGTACAGCATCCTTTATCTTTTCCGGCTCCTTTTTCA |
| | TGAAATAAAAAAATGCAAATTGTTTTCCCATGCCATCCTCCCTGCAG (SEQ ID NO: 57) |
| | 1 ttaaaatccg aagactggtc gggaaggtgt cccggatgca cggagtggaa tacctacagg |
| | 61 ctcgacgttt atggctcctg caaagcgtaa agacagcgat gagttatcag tgatgagtga |
| | 121 tgagttaaaa aacaaaaaaa aatcaaagac gaaaagtaaa agctcaaaag caagcacatc |
| | 181 accgtcgtta aaagtcgtac tcgatacgag ccttttcgta aatccggacg tcaggacaag |
| | 241 cctcggagca acccccacag aggctttgga aacattcctt tttctggcgg cgcagataca |
| | 301 tatactcgaa ttctacatgc ctccgtcgat attcgaagaa ctcctgcatt ttgtggagag |
| | 361 ggacaagata ccggccgatc tccttcttat cttgcatcag aaaccgccta aaaaacatga |
| | 421 actcacgact cccgcctttc tcttatacga attgattgag gacataaggg aaagggtgaa |
| | 481 caaggggctg cggatagcag aaaaggccgt aaggagtaca gatcaaaaaa agacggatga |
| | 541 aatcgttcag gatatgagaa gaaagtacag ggacgcgctt agagaaggga tcatcgacag |
| | 601 caaggaagac gtcgatctcc tgctgctcgc catggaactt gacgccctgc ttatcacggc |
| | 661 cgatcagggg ctgataaagt gggccgaaaa attaggtgta agatggctgt tcccggagaa |
| | 721 attcaaggaa tatttaatga gcgcgataaa aagaaccgaa cttctttccg caaaagaata |
| | 781 aagtgtagaa acaatgtagt tttgggaaga ttctttgatg gaaaaagaac ttgttttagg |
| | 841 catcgattat gggggcaaat acacaggcct tggggtggtt gaccgcagga acaatcaggt |
| | 901 gttgtatgcg aggaccataa aaatgcgtga tgatgttgcc gatattctga aaggtcgtcg |
| | 961 tgaacagcgt ggtattcgtc gtacccagca gacccgtaaa aaacgtctgc gtgaactgaa |
| | 1021 aaagtatctg aacagcattg gctatgataa cagcaccgaa ctgttcaaaa gcatttatag |
| | 1081 cctggcacat aaacgcggtt atgattatgc agatatgccg acaccggaag aaattgaaga |
| | 1141 aatggacgag aaagaaaaaa aagaatggaa agatacccag cgcaatagcc gttatcgtaa |
| | 1201 agaagttctg gcagacgttc gtaaagttat gagcaatggt ggtgcaagtg atgagcagat |
| | 1261 taaacgtgtt gaaagcatct tcaacaaaca gtatcgtccg aaacgtttta acaatcgcat |
| | 1321 tctgaccaaa tgcaaagtgt gcggtaaaaa tacaccgctg cgtcgtaatg ttcgtgaact |
| | 1381 gctgctggaa aatattgttc gttttctgcc gctggaaagc gaactgaaag aaaccctgaa |
| | 1441 acgtaccatt ctggaaggtc agcagggtaa tatcaataaa ctgtttcgca agctgaaatt |
| | 1501 caaccagaaa gattggcctg gtaaaaacct gaccgatatt gccaaaaaca aactgcctgg |
| | 1561 tcgtctgccg ttttgcaaag aacattttgc agagaacgaa aaattcacca ccatcgaaaa |
| | 1621 aagcaccttt cgtctggcac cgagcctgaa aaccaaaatt gaaaatgtgc tgaccgtgat |
| | 1681 caaagatgaa gtgatgccga attttgcact ggatcgtgtt gttatggaaa gcaacaattt |
| | 1741 tgacattgca gccaaaacca aaggcaaaaa acgcctggca aaagaagaat atagcaaggg |
| | 1801 ccataaagaa aatcgtgaaa cactgatgga aagcctgctg cgtgaaaccg atggtcgttg |
| | 1861 tgtttattgt ggtaaaacca ttacactggc cgatgccaac aaagatcata tctatcctaa |
| | 1921 aaaagccggt ggcagcaaca tttttgcaaa tctggttgca tgttgtcgca gctgcaatga |
| | 1981 aaataaaggt ggtcgtaccc cgagcgaaag cggtattatg ccgaatcctg aagttgttgc |
| | 2041 aaccatcaaa aacgatctga aaaagaagat cctggacgac gcacgtagca ttaaacagct |
| | 2101 ggatttcaac aaatatatga gccatgcaag cattggttgg cgtcatatgc gtgatcgtct |
| | 2161 gaaagaactg accggtaatg ataaactgcc ggttgaacgt ctgagcggta ttgttaccgc |
| | 2221 ctattttcgt cgttggtggg gtttcaaaaa agaacgtgca aacgataaac atcatgccct |
| | 2281 ggatgcagtt attctggcaa gccgtaaaga ttataccgat gaaggtctgg tggcaatgac |
| | 2341 actgaaaccg gcaaatagtg atggtcgcga atttgatccg gaaaaacata tcaaagaaag |
| | 2401 cgaagagttc aaacgcaata aaggtagccg tggtagcgca ctgtatgata aaaatccgct |
| | 2461 gagcattaaa aacggcaaaa ttgcacgtcg ttatatggtg accgaaattg aacgtggtaa |
| | 2521 agaagatgcc gttattagcg aagaatggcg tgaaaagctg aaagaagcat ttgatcgttt |
| | 2581 tggtgtgagt aatggtaaat gcctgacaga tctgcagacc aaagaagttg gtctgtacgg |
| | 2641 tcagaaaaat ccgatgtcac tgaaatgtgc agttcgtggt gcaggtaaag gtcagattgt |
| | 2701 tctgattggt aacaacgcct ttaaaaccaa tgtgcataat gttggtgttg ccgtgtatct |
| | 2761 ggatgaaaaa ggtaaaaagc gtgcatgcga gctgaaaaat cagcgtctgg caaaacattt |
| | 2821 tgttgaaccg caggatgaga ttaaaggcaa gattctgttt accctgcgta aaggtgatac |
| | 2881 cgttaaagca gaagatggta acatttaccg catacttgaa cttggcgaaa ggcctgttgt |
| | 2941 agacataaag tgggtaccaa catctgatgg caagaaaaag agagtaaaga cagctataca |
| | 3001 cgcaacaaag ctcacaaagc tctaactgaa aattttttta atgtcctaac ccacaatgaa |
| | 3061 ggtgtcatta tcgactgtag ttttagtttg gcttaggctg gacaaaccca agcgtacatt |
| | 3121 ccttcatcat ataggcgttt tacaattaag gctctttttg attttcgcaa accccctggg |
| | 3181 gtttatttca aaagatggct tttagaaaat tattgaaaat tttctctgaa aacttcttga |
| | 3241 aaatactaag ctataaggac ggtttcaatc atcattttca aagagcctta actgtaatga |
| | 3301 gaagtcattt aataaggcca ctgttaaaaa gcgtttcaat catcattttc aaagagcctt |
| | 3361 aactgtaacg ctcatataag caaggtctgt ccgccggaga ggacggtgtt ttctccatcg |
| | 3421 gtcgcgctac tgaaccaact gactgtcagt tagacgaaac aatcagaaca gagtccctgc |
| | 3481 cggaaaccgt ttccggttgt ccagcgaaca ttgagaaagc tgcgacgcca gatgcacctt |
| | 3541 ccggtgggaa gtcctccgct caaacgcctg aagcgtttcg cagcctttct gaaacaaatc |
| | 3601 aaaaaagctg agacaagatt cctttaaaat agagcatctc aaagaaaagt ccctgcgagc |
| | 3661 aatgcgcttc ttgaaggaag cccctctcta acgtccgaaa cgctcaggga cactaaaata |
| | 3721 tatctcagaa ttgtaaaaga gcctaaacta taatatgatt acattatatg cctttaaatt |
| | 3781 tcaagagctt tagcggaggt ggatatgcaa agcaatactt ttaaaatttc acgcatccat |
| | 3841 cctgaatacg tgcctaaaga aaaattgcct cttgcccaat tcaatctttt tacaattagg |
| | 3901 tcgataatcc agatgcaggc cagaaattta gcggcctttc tgaacggcaa aagagaatac |
| | 3961 aaaacgttca gatttaagtg gtaattgatg gaagaacata cagtctatat tttttacgat |
| | 4021 atagaagatg acggcatacg caataagatt gcggagaagt gtaaagacta tgggctcgaa |
| | 4081 agaatacagt tcagcggctt ttccggcatt ttaaatagaa acaagcggga agaactattt |
| | 4141 ctaaggctca tttctttaat tggcggcaaa tcaggcaaaa ttcttatgct tcccttatgt |
| | 4201 gaaagggata cgaaggtaag aagggagttt atacaggaag gacaagatga ttcaactgag |
| | 4261 ggtcggtgat ctcaagcagt tcgtatattg tccaagagtc gtgttctaca attatgtgat |
| | 4321 gcctgtcgag aaagttccaa ccttcaagat ggagcatggg aagatagcag aggataaaat |
| | 4381 agacaggctt gagcacagaa gaaaacttaa aaagtacggc ttgtccggcg gggaaaggga |
| | 4441 attccacttg aatttatatt ctccgaaata tgagttatca ggcaagattg atctgcttat |
| | 4501 caatactgtc ggttcttatt atccggttga ctttaaatat accacctcgc agccgcataa |
| | 4561 aaatcatatc tatcaacttt tggggtatgc cctaatactt gaagacatat ataaatgcaa |
| | 4621 agtcaatagg ggatttgtat atataattcc caaagaggat gcaatttctt ttgacttaac |
| | 4681 ggatgatttg aagggcaaag cagaaatttt gcttgatgag atcagagata tgatagcatt |
| | 4741 gcagcaaatg cctgcccctg caaactccag aaataaatgt gacgactgcg aatataggaa |
| | 4801 tttctgcgga gatatttttt gacaattata aaccgccgag ggctttatac agccctatca |
| | 4861 cgaattccgt catcctctcg taatcgagcg tttcgaatgt atctcccggc gcatg |
| | (SEQ ID NO: 58) |

Sequences of 4 exemplary Cas proteins are shown in Table 12 below.

TABLE 12

| | | | Spacer | | Protein |
|---|---|---|---|---|---|
| Subtype | Source | DR | Length | Protein Sequence | Length |
| Type II- | Gammaproteo- | GTTGGATCA | 30 | MKQLTLDVGIASIGWAIVSKKGNVKAGSRIFPDAKSGRESNQS | 807 |
| C | bacteria | GGACGTCAC | | RRAARLMRRGYRRKAKRRADTLAIIRSIHPGFDPEGHPDIEREA | |
| | bacterium | TTTTCGAAT | | LIKAIITPGAPTPSLDQLACAFQRFAKSRWPQYSRTLPKRTERED | |
| | AqS3 | TCGAAAAC | | QFIQVWVIAERTYPDRFTPDVATRLLQAIFFQRPIKDGDRAKCQ | |
| | | (SEQ ID | | LFRHHGDKAPLVGWTHEPELQRFAILSDLSNLTIGIGSTDNLLC | |
| | | NO: 59) | | EYPDIIEDLETRCFETGMSWREIAEHVKEVIGKGVVFRGIDGQK | |
| | | | | KVGRNGIGPAKLETIDEEGNSTKSTASMSVEAAVMIYHQMKA | |
| | | | | DRCRAATAKKTLIDAGALSAPLTAKDIKRGDRTLTITELMDMA | |
| | | | | GRITDPTIRAIYHQVEMLVNELIARFGKPERIVIEAQKEIGRSI | |
| | | | | EDIEKAMAREREKHIERQRENRARNAAMGTKARFARLCAIRGDR | |
| | | | | CFISDRPAAEVGHLIADSIGGTLEMANLIPIDPAINKEMGNRTPY | |
| | | | | EAFRKTEYWSIIQRKLQALEDEVKALKPPKGTKGTAWTIYHRA | |
| | | | | KHQFDFFAWRFQSNARETHQRNFRPGSLDDLRWIENLLFLGVA | |
| | | | | PICDNIRIVSGRTTERIRREILGMDKDRRDHRHHALDALAIMLA | |
| | | | | NPLKPWDLKSSNSLGIPLGRIKQAFADAVVSQKQDHSLRTALH | |
| | | | | KENAIPKTKRGAAYRKIGTGASERVVDTQSKAYCEVWALPNG | |
| | | | | KWEAVVVSSFDAAQKNYRQGIDHRPHPAARLVMRLFKSDLLG | |
| | | | | IGGKIYRVQELLGSGSIYLVDHRFAGTIRDARAVCKTGVNVDFF | |
| | | | | SKGGDSLRKAGARLVSIRKSWVGS (SEQ ID NO: 60) | |
| Type II- | Deltaproteo- | GTTTCACGC | 36 | MERELVLGIDYGGKYTGLAVVDRRHNQVLYANRLKMRDDVAG | 747 |
| B | bacteria | CATATTTTC | | ILKDRRKQRGIRRTAQTKKKRLRELKNYLKSIGYNESTATFET | |
| | bacterium | AAAGAGCC | | VYSLAHKRGYDYADMPEEKTSEEIEAMDVEERKQWEKEKQE | |
| | GWF2_ | TTAACTGTA | | WEETKRNSRHRKEVVKDVHKAMIEGRATEEQIKRVERIFNKQ | |
| | 4212 | AC | | YRPKRFNNRILTKCKVEDCGVNTPLRKNVRDLLIENIVRFFPIEQ | |
| | | (SEQ ID | | SEKDNLKDAVLDKNRREEVKSFFRKHKTDEHIRKQVYDIADN | |
| | | NO: 61) | | KLSGRTVFCKEHILEHTEHSKEERKVFRLAPSLKTKIENVLAVI | |
| | | | | KDEILPKFTVNKVVMESNNFDIAAKTQGKKRLAKEEYGKGPRE | |
| | | | | GKETRKEALLRETDGRCIYCGKSIDISNAHDDHIFPRKAGGLNIF | |
| | | | | ANLVACCAVCNENKKGRTPLESGISPKPEIIAFMKNDLKKKILE | |
| | | | | DARNINTVDFNKYMSHASIGWRYMRDRLRESAGNKKLPIERQ | |
| | | | | SGIYTAYFRRWWGFKKERGNTLHHALDAVILASRKGYSDDGL | |
| | | | | VDMTLKPKYNKGGGFDSEKHLPEPIEFKRDKGSKGSALHDRNP | |
| | | | | LSYKKGIITRRFMVTEIECGKEDDVISETYREKLKEAFKRFDTK | |
| | | | | KGKCLTDKEAKEAGFCIKKNELVMSLKCSIKGTGPGQMIRINN | |
| | | | | NVFKTNVHNVGVDVYLDEKGKKKAYERKNPRLSKHFIEPPPQ | |
| | | | | PNGRVSFTLKRRDMVTVEGEDAIYRIKKLGTSPTIEAVVGSDG | |
| | | | | KTRTVSATKLTKANSAE (SEQ ID NO: 62) | |
| Type II- | JGI | GTTTCACGC | 35 | MEREIVLGIDYGGKYTGLAVVDRRHNQVLYANRVKMRDDVA | 778 |
| B | Metagenome: | CATATTTTC | | DILKGRREQRGIRRTAQTKKKRLRELRNYLKSIGYNESTATFKT | |
| | IMG | AAAGAGCC | | VYSLAHKRGYDYADMPEEKTSEKIEAMDEEERKQWEKEKQE | |
| | 3300025 | TTAACTGTA | | WEETKRNSRHREEVLKDVRKAMTEGRAAEEQIKRVESIFNKQ | |
| | 323 | AC | | YRPKRFNNRILTKCKVCGKNTPLRRKVRELLLENIVRFLPLESE | |
| | | (SEQ ID | | MKETLKQTILEGQQENINKIFRKLKFNQKDWPGKNLTDIAKNKL | |
| | | NO: 63) | | PGRLPFCKEHFSENEKFTMIEKSTFRLAPSLKTKIENVLAVIKD | |
| | | | | EILPKFTINKVVMESNNFDIAAKTQGKKRLAKEEYGKGPREGK | |
| | | | | ETRKEALLRETNGRCIYCGKSIDISNAHDDHIFPRKSGGLNIFA | |
| | | | | NLVACCAVCNENKKGRTPLESGILPKPDVIRFMEDEFNRLKIKM | |
| | | | | WKIRRKQSLTTEEIMDKAKRNKLELKLKILEDAQQVNELDFNK | |
| | | | | YMSHASIGWRYMRDRLRESAGNKKLPIERQSGIYTAYFRRWW | |
| | | | | GFKKERGNTLHHALDAVILASRKGYSDDGLVDMTLKPKYNKGG | |
| | | | | EFDPEKHIPEPIEFKRDKGSKGSALHDRNPLSYKKGIITRRFMV | |
| | | | | TEIECGKEDDVISETYREKLKEAFKLFDTKKGKCLTDKEAKEA | |
| | | | | GFCIKKHELVMSLKCSIKGTGPGQMIRINNNVFKTNVHNVGVD | |
| | | | | VYLDEKGKKKAYERKNPRLSKHFIEPPPQPNGRVSFTLKRRDM | |
| | | | | VTVEGEDAIYRIKKLGTSPTIEAVVGSDGKTRTVSATKLTKANN | |
| | | | | AE (SEQ ID NO: 64) | |
| Type II- | Nitrospirae | GTTACAGTC | 35 | MEKELVLGIDYGGKYTGLAVVNQKNNQVLYARTVKMRDDVT | 762 |
| B | bacterium | GTTGTTTTC | | DILAGRREQRSLRRTLQTKKKRLRELKNYLESIGGIYEESSGTFT | |
| | RBG_1 | AAAGAGCC | | IEPFRTVYSLAHKRGYDYADLPEEKTSEEIEAMDAKERKQWEK | |
| | 3_39_12 | TTAACTGTA | | EKKELEETQRNSRHRDEVLRDVRNVMTEGNLSEEQIIKVESIFN | |
| | | AC | | KQYRHKRFNNRILTKCKVCGKNTPLRINVRELLLENIVRYLPLQ | |
| | | (SEQ ID | | NKERELLKLTILKGHQQDINEIFKHFRKVYKITLNQKDWPGKN | |
| | | NO: 65) | | LIDIARNQLRGRLLFCKVHFPENEKYVSIEKKTFRLAPSLKTKIE | |
| | | | | NVLSVIKDDILPNFTLNNVVMESNNFDIAAKTKGKKRLLKEEY | |
| | | | | SKGHRESGETRKEALLRETDSRCIYCGKGIDLSNAHEDHIFPRK | |
| | | | | AGGINIFGNLVACCSVCNEEKRGRTPLESGILPKPEIVSFITNDL | |
| | | | | KKKILEDAQYINTLDFNKYMSHASIGWRHMRDRLRELTGNKE | |
| | | | | LLIKRQSGIYTAYFRKWWGFIKERGNHGHHALDAVILASKKSY | |
| | | | | AEDGKVDMTIKPCGEDGKEFDIERHLSEMKEFRRDKGGKSAPL | |
| | | | | HDRNPLSFKNDIITRRFMVTEIECGKEAVIISEEYRKKLTEAFKR | |
| | | | | FGIAKGKYLTDEQAKDAGFYLRKNGEGVMSLKCEVKGTGYN | |
| | | | | QMIRIKNNIFKTNVHNVGVAVFLDEKGKKRACELKNPRLSKHF | |
| | | | | VKPAEQVKGKVIFILKRGNMVTVEGEEMIYRVKKLGTSPVIEAI | |
| | | | | VGSDGKTRTVSATKLLKINHTKKV (SEQ ID NO: 66) | |
| Type II- | Nitrospiraceae | GTTTCAATC | 35 | MEKELVLGIDYGGKYTGLGVVDRRNNQVLYARTIKMRDDVA | 735 |
| B | bacterium | ATCATTTTC | | DILKGRREQRGIRRTQQTRKKRLRELKKYLNSIGYDNSTELFKS | |
| | isolate | AAAGAGCC | | IYSLAHKRGYDYADMPTPEEIEEMDEKEKKEWKDTQRNSRYR | |
| | UBA99 | TTAACTGTA | | KEVLADVRKVMSNGGASDEQIKRVESIFNKQYRPKRFNNRILT | |
| | 35 | A | | KCKVCGKNTPLRRNVRELLLENIVRFLPLESELKETLKRTILEG | |
| | | (SEQ ID | | QQGNINKLFRKLKFNQKDWPGKNLTDIAKNKLPGRLPFCKEHF | |
| | | NO: 67) | | AENEKFTTIEKSTFRLAPSLKTKIENVLTVIKDEVMPNFALDRV | |
| | | | | VMESNNFDIAAKTKGKKRLAKEEYSKGHKENRETLMESLLRE | |
| | | | | TDGRCVYCGKTITLADANKDHIYPKKAGGSNIFANLVACCRSC | |
| | | | | NENKGGRTPSESGIMPNPEVVATIKNDLKKKILDDARSIKQLDF | |
| | | | | NKYMSHASIGWRHMRDRLKELTGNDKLPVERLSGIVTAYFRR | |
| | | | | WWGFKKERANDKHHALDAVILASRKDYTDEGLVAMTLKPAN | |
| | | | | SDGREFDPEKHIKESEEFKRNKGSRGSALYDKNPLSIKNGKIAR | |
| | | | | RYMVTEIERGKEDAVISEEWREKLKEAFDRFGVSNGKCLTDLQ | |
| | | | | TKEVGLYGQKNPMSLKCAVRGAGKGQIVLIGNNAFKTNVHNV | |
| | | | | GVAVYLDEKGKKRACELKNQRLAKHFVEPQDEIKGKILFTLRK | |
| | | | | GDTVKAEDGNIYRILELGERPVVDIKWVPTSDGKKKRVKTAIH | |
| | | | | ATKLTKL (SEQ ID NO: 68) | |

### Example 2

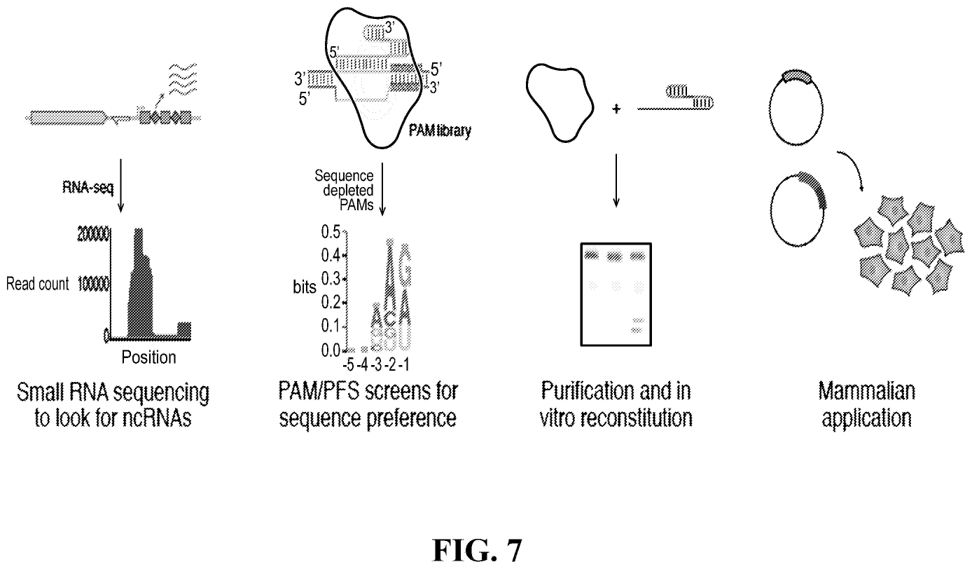

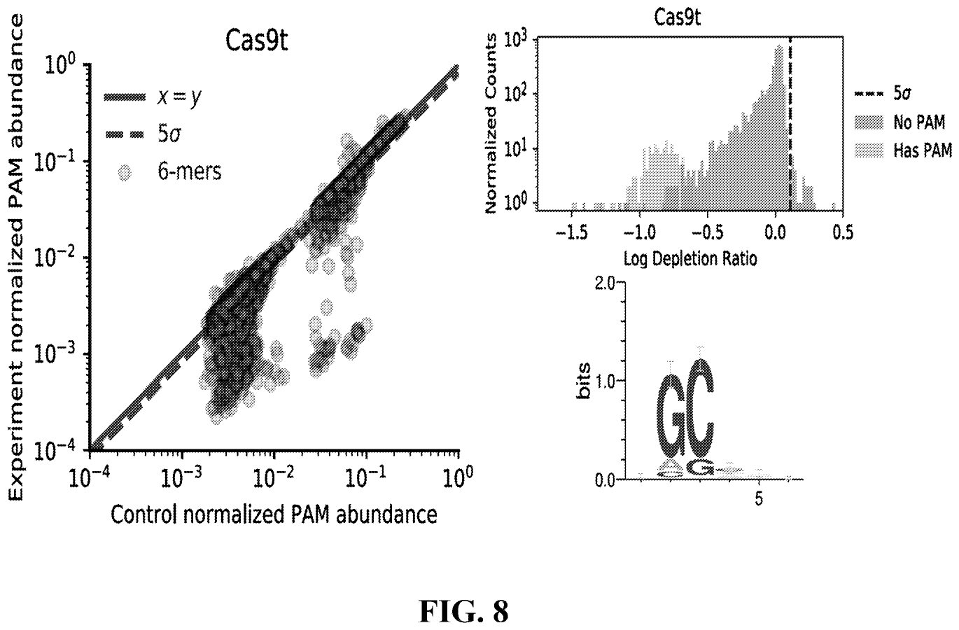

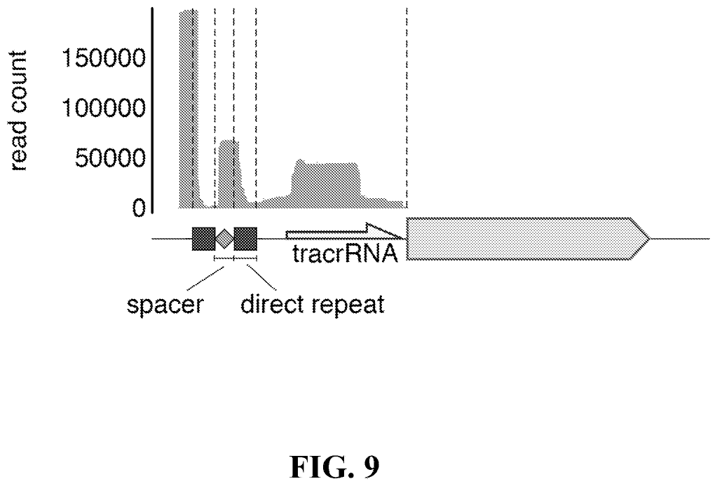

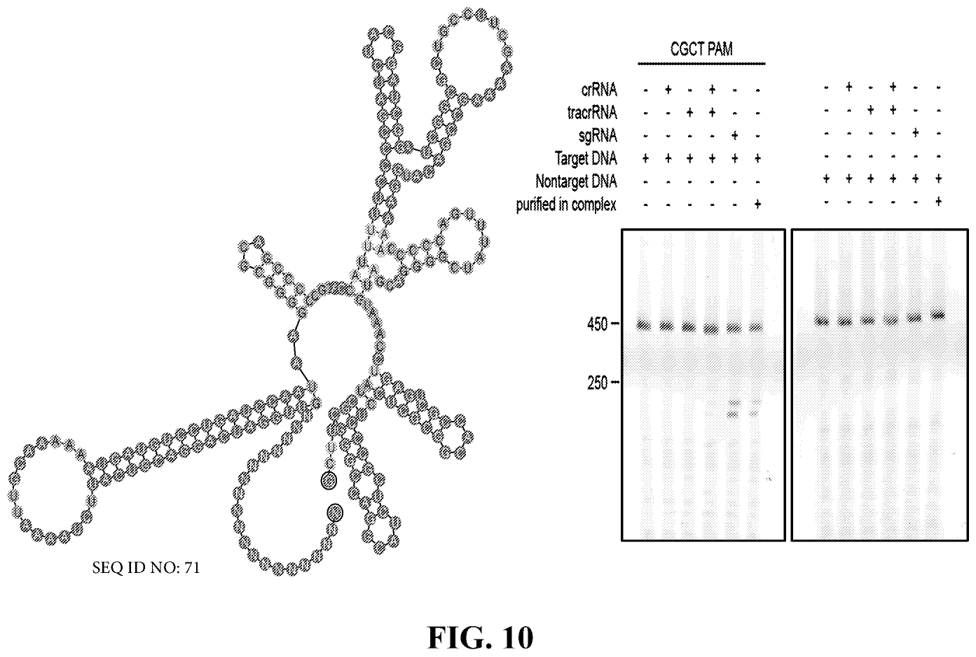

FIG. 7 shows an exemplary method of identifying and characterizing Cas proteins. Applicants tested the interference activity showed that an exemplary Cas9-t had interference activity with NGCH PAM (FIG. 8). Pulldown of the Cas9-t protein bound to ncRNAs revealed processed CRISPR and tracrRNA (FIG. 9). The Cas9-t cleaved dsDNA in vitro using an sgRNA (FIG. 10).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A non-naturally occurring or engineered composition comprising:
a Cas protein that
comprises a RuvC domain and a HNH domain, and
is less than 850 amino acids in size; and
a guide sequence capable of forming a complex with the Cas protein and directing the complex to bind to a target sequence.

2. The composition of claim 1, wherein the Cas protein is a Type II Cas protein.

3. The composition of claim 2, wherein the Type II Cas protein is a Type II-B Cas protein.

4. The composition of claim 2, wherein the Type II Cas protein is a Type II-C Cas protein.

5. The composition of claim 2, wherein the Type II Cas protein is Cas9 or an ortholog thereof.

6. The composition of claim 1, wherein the Cas protein is a protein from Table 12.

7. The composition of claim 1, wherein the Cas protein is derived from Gammaproteobacteria bacterium AqS3, Deltaproteobacteria bacterium GWF2_42_12, JGI Metagenome: IMG 3300025323, Nitrospirae bacterium RBG_13_39_12, or Nitrospiraceae bacterium isolate UBA9935.

8. The composition of claim 1, wherein the composition comprises two or more guide sequences capable of hybridizing to two different target sequences or different regions of a target sequence.

9. The composition of claim 1, wherein the guide sequence is capable of hybridizing to one or more target sequences in a prokaryotic cell.

10. The composition of claim 1, wherein the guide sequence is capable of hybridizing to one or more target sequences in a eukaryotic cell.

11. The composition of claim 1, wherein the Cas protein comprises one or more nuclear localization signals.

12. The composition of claim 1, wherein the Cas protein comprises two or more nuclear localization signals.

13. The composition of claim 1, wherein the Cas protein comprises one or more nuclear export signals.

14. The composition of claim 1, wherein the Cas protein is catalytically inactive.

15. The composition of claim 1, wherein the Cas protein is a nickase.

16. The composition of claim 1, wherein the Cas protein is associated with one or more functional domains.

17. The composition of claim 16, wherein the one or more functional domains comprises one or more heterologous functional domains.

18. The composition of claim 16, wherein the one or more functional domains cleaves the target sequence.

19. The composition of claim 16, wherein the one or more functional domains modifies transcription or translation of the target sequence.

20. The composition of claim 16, wherein the one or more functional domains comprises one or more transcriptional activation domains.

21. The composition of claim 20, wherein the one or more transcriptional activation domains comprises VP64.

22. The composition of claim 16, wherein the one or more functional domains comprises one or more transcriptional repression domains.

23. The composition of claim 22, wherein the one or more transcriptional repression domains comprises a KRAB domain or a SID domain.

24. The composition of claim 16, wherein the one or more functional domains comprises one or more nuclease domains.

25. The composition of claim 24, wherein the one or more nuclease domains comprises Fok1.

26. The composition of claim 16, wherein the one or more functional domains has one or more of the following activities: methylase activity, demethylase activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity and nucleic acid binding activity.

27. The composition of claim 1, further comprising a recombination template.

28. The composition of claim 27, wherein the recombination template is inserted by homology-directed repair (HDR).

29. The composition of claim 1, further comprising a tracr RNA.

30. The composition of claim 1, wherein the Cas protein is a chimeric protein comprising a first fragment from a first Cas protein and a second fragment from a second Cas protein.

31. The composition of claim 1, further comprising a nucleotide deaminase or a catalytic domain thereof.

32. The composition of claim 31, wherein the nucleotide deaminase is an adenosine deaminase.

33. The composition of claim 31, wherein the nucleotide deaminase is a cytidine deaminase.

34. The composition of claim 31, wherein the nucleotide deaminase or catalytic domain thereof is covalently or non-covalently linked to the Cas protein or the guide sequence, or is adapted to link thereof after delivered to a cell.

35. The composition of claim 31, wherein the nucleotide deaminase or catalytic domain thereof has been modified to increase its activity against a DNA-RNA heteroduplex.

36. The composition of claim 31, wherein the nucleotide deaminase or catalytic domain thereof has been modified to reduce off-target effects.

37. The composition of claim 31, wherein the composition is capable of modifying one or more nucleotides in the target sequence.

38. The composition of claim 37, wherein modification of the one or more nucleotides in the target sequence remedies a disease caused by a G→A or C→T point mutation or a pathogenic SNP.

39. The composition of claim 37, wherein the disease is cancer, haemophilia, beta-thalassemia, Marfan syndrome, or Wiskott-Aldrich syndrome.

40. The composition of claim 37, wherein modification of the one or more nucleotides in the target sequence remedies a disease caused by a T→C or A→G point mutation or a pathogenic SNP.

41. The composition of claim 37, wherein modification of the one or more nucleotides at the target sequence inactivates a gene.

42. The composition of claim 37, wherein modification of the one or more nucleotides modifies gene product encoded at the target sequence or expression of the gene product.

43. The composition of claim 1, further comprising a reverse transcriptase or functional fragment thereof.

44. A non-naturally occurring or engineered composition comprising one or more polynucleotide sequences encoding:
a Cas protein that
comprises a RuvC domain and a HNH domain, and
is less than 900 amino acids in size; and
a guide sequence capable of forming a complex with the Cas protein and directing the complex to bind to a target sequence.

45. The composition of claim 44, wherein the one or more polynucleotide sequences are codon optimized to express in a eukaryote.

46. The composition of claim 44, wherein the one or more polynucleotide sequences is mRNA.

47. The composition of claim 44, wherein the one or more polynucleotide sequences further encode a reverse transcriptase or functional fragment thereof.

48. A vector system comprising the one or more polynucleotides sequences of any one of claims 44 to 46.

49. The vector system of claim 48, comprising:
a first regulatory element operably linked to the polynucleotide sequence encoding the Cas protein; and
a second regulatory element operably linked to the polynucleotide sequence encoding the guide sequence.

50. The vector system of claim 49, wherein the first and/or second regulatory element is a promoter.

51. The vector system of claim 50, wherein the promoter is a minimal promoter.

52. The vector system of claim 51, wherein the minimal promoter is Mecp2 promoter, tRNA promoter, or U6 promoter.

53. The vector system of claim 48, which is comprised in a single vector.

54. The vector system of claim 48, wherein the one or more vectors comprises viral vectors.

55. The vector system of claim 48, wherein the one or more vectors comprises retroviral, lentiviral, adenoviral, adeno-associated, or Herpes simplex viral vectors.

56. A delivery system comprising the system of any one of claims 1 to 55 and a delivery vehicle.

57. The delivery system of claim 56, wherein the delivery vehicle comprises lipids, sugars, metals, proteins, liposomes, nanoparticles, exosomes, microvesicles, nucleic acid nanoassemblies, a gene gun, an implantable device, or a vector system.

58. The delivery system of claim 56, wherein the delivery vehicle comprises ribonucleoproteins.

59. A cell comprising the composition of any one of claims 1 to 55.

60. The cell of claim 59, wherein the cell is a eukaryotic cell, a human or non-human animal cell, a therapeutic T cell, antibody-producing B-cell, a stem cell, or a plant cell.

61. A tissue, organ, or organism comprising the cell of claim 59.

62. A cell product from the cell of claim 59.

63. A method of modifying one or more target sequences, the method comprising contacting the one or more target sequences with a composition of any one of claims 1 to 55.

64. The method of claim 63, wherein the composition further comprises a recombination template, and wherein modifying the one or more target sequences comprises insertion of the recombination template or a portion thereof.

65. The method of claim 63, wherein the one or more target sequences is in a prokaryotic cell.

66. The method of claim 63, wherein the one or more target sequences is in a eukaryotic cell.

67. The method of claim 63, wherein the one or more target sequences is comprised in a nucleic acid molecule in vitro.

68. A cell obtained from the method of any one of claims 63-67.

69. The cell or progeny thereof of claim 68, wherein the cell is a eukaryotic cell, a human or non-human animal cell, a therapeutic T cell, antibody-producing B-cell, a stem cell, or a plant cell.

70. A non-human animal or plant comprising the modified cell or progeny thereof of claim 68 or 69.

71. A modified cell or progeny thereof of claim 68 or 69 for use in therapy.

72. A method of treating a disease, disorder, or infection comprising administering an effective amount of the composition of any one of claims 1 to 55 in a subject in need thereof.

73. A method of producing a plant having a modified trait of interest encoded by a gene of interest, the method comprises contacting a plant cell with a composition of any one of claims 1 to 55, thereby either modifying or introducing the gene of interest, and regenerating a plant from the plant cell.

74. A method of identifying a trait of interest in a plant, the trait of interest encoded by a gene of interest, the method comprises contacting a plant cell with a composition any one of claims 1 to 55, thereby identifying the gene of interest.
